(19) United States
(12) Patent Application Publication
(10) Pub. No.: US 2025/0017103 A1
(43) Pub. Date: Jan. 9, 2025

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: DuPont Specialty Materials Korea Ltd., Chungcheongnam-do (KR)

(72) Inventors: Young-Kwang KIM, Gyeonggi-do (KR); Yoo-Jin DOH, Gyeonggi-do (KR); Chi-Sik KIM, Gyeonggi-do (KR); HaeYeon KIM, Gyeonggi-do (KR)

(21) Appl. No.: 18/732,706

(22) Filed: Jun. 4, 2024

(30) Foreign Application Priority Data
Jun. 21, 2023 (KR) 10-2023-0079590
May 21, 2024 (KR) 10-2024-0065554

(51) Int. Cl. H10K 85/60; C09K 11/02; C09K 11/06; H10K 85/20

(52) U.S. Cl. CPC H10K 85/653; C09K 11/02; C09K 11/06; H10K 85/20; H10K 85/615; H10K 85/6574; H10K 85/6576

(57) ABSTRACT

The present disclosure relates to a plurality of host materials, an organic electroluminescent compound, and an organic electroluminescent device comprising the same. By comprising the specific combination of the compound according to the present disclosure as a plurality of host materials and/or an organic electroluminescent compound according to the present disclosure as a host material, an organic electroluminescent device can be provided which has low driving voltage and/or high luminous efficiency and/or long lifespan characteristics, compared to conventional organic electroluminescent devices.

### TECHNICAL FIELD

The present disclosure relates to a plurality of host materials, an organic electroluminescent compound, and an organic electroluminescent device comprising the same.

### BACKGROUND ART

The TPD/Alq₃ bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. In many applications such as TVs and lightings, OLED lifetime is insufficient, and high OLED efficiency is still required. Typically, the higher the luminance of an OLED becomes, the lifetime of the OLED shortens correspondingly. Therefore, an OLED having high luminous efficiency and/or long lifespan characteristics is required for long-term use and high display resolution.

Various materials or concepts have been proposed for the organic layer of an organic electroluminescent device in order to improve luminous efficiency, driving voltage, and/or lifespan, but these have not been satisfactory for practical use. In addition, there is a continuous need to develop organic electroluminescent devices with improved performance, such as improved driving voltage, luminous efficiency, power efficiency, and/or lifespan characteristics compared to previously disclosed organic electroluminescent devices.

However, Korean Patent Application Laid-open No. 2017-0137976 and WO 2021/025163 A1 disclose an anthracene derivative substituted with benzoxanthene or benzothioxanthene as a host material for the light-emitting layer, but do not specifically disclose the specific combination of host materials described in the present disclosure.

### DISCLOSURE OF THE INVENTION

### Technical Problem

The object of the present disclosure is, firstly, to provide a plurality of host materials capable of producing an organic electroluminescent device having low driving voltage and/or high luminous efficiency and/or long lifespan characteristics and, secondly, to provide an organic electroluminescent compound suitable for use as an organic electroluminescent device. In addition, another object of the present disclosure is to provide an organic electroluminescent device with low driving voltage and/or high luminous efficiency and/or long lifespan characteristics by comprising a specific combination of compounds according to the present disclosure as a plurality of host materials and/or an organic electroluminescent compound according to the present disclosure as a host material.

### Solution to Problem

As a result of intensive studies to solve the technical problem above, the present inventors found that the aforementioned objective can be achieved by a plurality of host materials comprising at least one first host compound represented by the following Formula 1 and at least one second host compound represented by the following Formula 2 wherein the first host compound and the second host compound are different from each other, thereby completing the present invention.

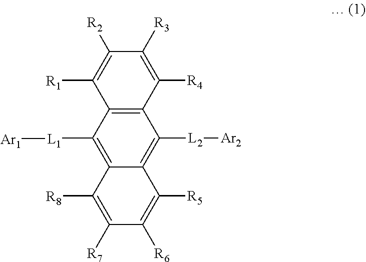

In Formula 1,
R₁ to R₈ each independently are selected from hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di-(C₁-C₃₀)alkylamino, a substituted or unsubstituted mono- or di-(C₂-C₃₀)alkenylamino, a substituted or unsubstituted mono- or di-(C₆-C₃₀)arylamino, a substituted or unsubstituted mono- or di-(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, and a combination thereof;
L₁ and L₂ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl, or a substituted or unsubstituted (3- to 30-membered)heteroaryl; and
Ar₂ is represented by the following Formula 1-A;

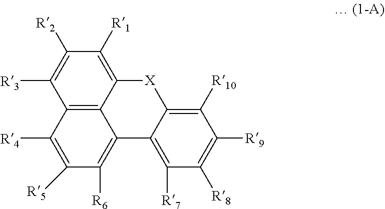

in Formula 1-A,
X represents —O— or —S—;
R′₁ to R′₁₀ each independently represent hydrogen, deuterium, a substituted or unsubstituted (C₆-C₃₀)aryl, or a substituted or unsubstituted (3- to 30-membered)heteroaryl; and
any one of R′₁ to R′₁₀ is linked to L₂;

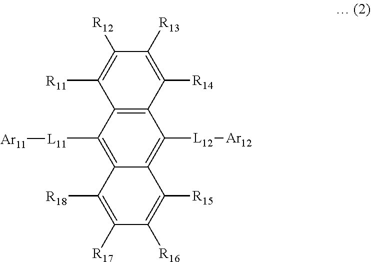

in Formula 2,
Ar₁₁ and Ar₁₂ each independently represent a substituted or unsubstituted (C₆-C₃₀)aryl or a substituted or unsubstituted (3- to 30-membered)heteroaryl;
L₁₁ and L₁₂ each independently represent a single bond, a substituted or unsubstituted (C₆-C₃₀)arylene, or a substituted or unsubstituted (3- to 30-membered)heteroarylene; and
R₁₁ to R₁₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of (C₃-C₃₀) aliphatic ring and (C₆-C₃₀) aromatic ring.

### Advantageous Effects of Invention

By comprising a plurality of host materials and/or an organic electroluminescent compound according to the present disclosure, an organic electroluminescent device having low driving voltage and/or high luminous efficiency and/or long lifespan properties can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials comprising at least one first host compound(s) represented by Formula 1 and at least one second host compound(s) represented by Formula 2, which is different from the first host compound, and an organic electroluminescent device comprising the same.

The present disclosure relates to an organic electroluminescent compound represented by Formula 11, an organic electroluminescent material comprising the organic electroluminescent compound, and an organic electroluminescent device comprising the organic electroluminescent compound and/or the organic electroluminescent material.

The present disclosure relates to an organic electroluminescent compound represented by Formula 12, an organic electroluminescent material comprising the organic electroluminescent compound, and an organic electroluminescent device comprising the organic electroluminescent compound and/or the organic electroluminescent material.

Herein, the term “organic electroluminescent compound” in the present disclosure means a compound that may be used in an organic electroluminescent device, and this may be comprised in any material layer constituting an organic electroluminescent device, as necessary.

Herein, the term “organic electroluminescent material” means a material that may be used in an organic electroluminescent device, and this may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (containing host and dopant materials), an electron buffer material, a hole-blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron-blocking layer, a light-emitting layer, an electron buffer layer, a hole-blocking layer, an electron transport layer, and an electron injection layer. As such, at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C₁-C₃₀)alkyl(ene)” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the “(C₃-C₃₀)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(C₆-C₃₀)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoran]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms and including at least one, preferably 1 to 4 heteroatom(s) selected from the group consisting of B, N, O, S, Si, P, Se, and Ge, in which the number of the ring backbone carbon atoms is preferably 5 to 25. The above heteroaryl(ene) may be a monocyclic ring, or a fused ring condensed with at least one benzene ring, and may be partially saturated. Also, the above heteroaryl herein may be one formed by linking at least one heteroaryl or aryl group to a heteroaryl group via a single bond(s). Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene), which has hole properties, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl. Herein, the term “a fused ring of (C₃-C₃₀) aliphatic ring and (C₆-C₃₀) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C₃-C₃₀) aliphatic ring and (C₆-C₃₀) aromatic ring may be replaced with at least one heteroatom selected from B, N, O, S, Si, and P, preferably at least one heteroatom selected from N, O, and S. The “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, and preferably this may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si, and P, preferably N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. Unless otherwise specified, the substituent may replace hydrogen at a position where the substituent can be substituted without limitation, and when two or more hydrogen atoms in a functional group are each replaced with a substituent, each substituent may be the same or different. The maximum number of substituents that can be substituted for a certain functional group may be the total number of valences that can be substituted for each atom forming the functional group. The substituted alkyl, the substituted alkenyl, the substituted aryl(ene), the substituted heteroaryl(ene), the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted fused ring of aliphatic ring and aromatic ring, the substituted mono- or dialkylamino, the substituted mono- or dialkenylamino, the substituted mono- or diarylamino, the substituted mono- or diheteroarylamino, the substituted alkylalkenylamino, the substituted alkylarylamino, the substituted alkylheteroarylamino, the substituted alkenylarylamino, the substituted alkenylheteroarylamino, and the substituted arylheteroarylamino in the formulas of the present disclosure each independently may be substituted with at least one selected from the group consisting of deuterium, halogen, cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (3 to 30-membered)heteroaryl unsubstituted or substituted with at least one (C₆-C₃₀)aryl, (C₆-C₃₀)aryl unsubstituted or substituted with at least one of (C₁-C₃₀)alkyl and (3- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, tri(C₆-C₃₀)arylgermanyl, amino, mono- or di-(C₁-C₃₀)alkylamino, mono- or di-(C₂-C₃₀)alkenylamino, mono- or di-(C₆-C₃₀)arylamino unsubstituted or substituted with (C₁-C₃₀)alkyl, mono- or di-(3- to 30-membered)heteroarylamino, (C₁-C₃₀)alkyl(C₂-C₃₀)alkenylamino, (C₁-C₃₀)alkyl(C₆-C₃₀)arylamino, (C₁-C₃₀)alkyl(3- to 30-membered)heteroarylamino, (C₂-C₃₀)alkenyl(C₆-C₃₀)arylamino, (C₂-C₃₀)alkenyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, di(C₆-C₃₀)arylboronyl, di(C₁—C₃₀)alkylboronyl, (C₁-C₃₀)alkyl(C₆-C₃₀)arylboronyl, (C₆-C₃₀)ar(C₁-C₃₀)alkyl, and (C₁-C₃₀)alkyl(C₆-C₃₀)aryl, etc.

When a substituent is not shown in the chemical formula or the compound structure of the present disclosure, it may signify that all positions that may be present as substituents are hydrogen or deuterium. That is, in the case of deuterium, an isotope of hydrogen, some of the hydrogen atoms may be deuterium, which is an isotope; and in this case, the content of deuterium may be 0% to 100%. In the case where the substituent is not shown in the chemical formula or the compound structure of the present disclosure, when deuterium is not explicitly excluded, hydrogen and deuterium may be mixed and used in the compound, such as when the content of deuterium is 0%, the content of hydrogen is 100%, and all substituents are hydrogen. The deuterium is an element having a deuteron composed of one proton and one neutron as an atomic nucleus, which is one of the isotopes of hydrogen, and may be represented by hydrogen-2, and the element symbol may be D or ²H. Although the isotope has the same atomic number (Z), an isotope having a different mass number (A) means the same number of protons and the number of neutrons may also be interpreted as an element having different numbers.

Herein, “combinations thereof” signifies that one or more components of the corresponding list are combined to form a known or chemically stable arrangement that a person skilled in the art could conceive of from the corresponding list. For example, alkyl and deuterium may be combined to form partially or entirely deuterated alkyl groups; halogen and alkyl may be combined to form halogenated alkyl substituents; and halogen, alkyl, and aryl may be combined to form halogenated arylalkyl. For example, preferred combinations of substituents may include up to 50 atoms excluding hydrogen and deuterium, or include up to 40 atoms excluding hydrogen and deuterium, or include up to 30 atoms excluding hydrogen and deuterium, or in many cases, preferred combinations of substituents may include up to 20 atoms excluding hydrogen and deuterium.

In the formula of the present disclosure, when multiple substituents are indicated by the same symbol, each of these substituents represented by the same symbol may be the identical or different from one another.

Hereinafter, the plurality of host materials according to one embodiment will be described.

The plurality of host materials according to one embodiment comprise at least one first host compound and at least one second host compound, wherein the first host compound is represented by the following Formula 1, the second host compound is represented by the following Formula 2, and the first host compound and the second host compound are different from each other.

The first host compound as the host material according to one embodiment may be represented by the following Formula 1.

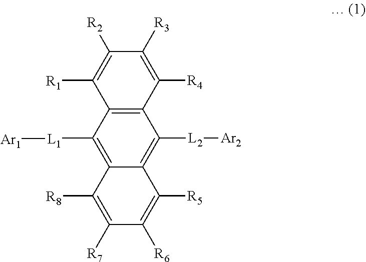

In Formula 1,
R₁ to R₈ each independently are selected from hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di-(C₁-C₃₀)alkylamino, a substituted or unsubstituted mono- or di-(C₂-C₃₀)alkenylamino, a substituted or unsubstituted mono- or di-(C₆-C₃₀)arylamino, a substituted or unsubstituted mono- or di-(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, and a combination thereof;
L₁ and L₂ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl, or a substituted or unsubstituted (3- to 30-membered)heteroaryl; and
Ar₂ is represented by the following Formula 1-A;

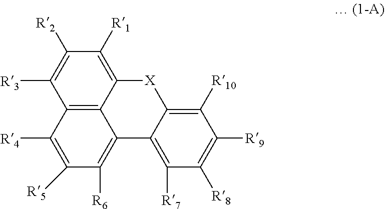

in Formula 1-A,
X represents —O— or —S—;
R′₁ to R′₁₀ each independently represent hydrogen, deuterium, a substituted or unsubstituted (C₆-C₃₀)aryl, or a substituted or unsubstituted (3- to 30-membered)heteroaryl; and
any one of R′₁ to R′₁₀ is linked to L₂.

In one embodiment, X may be —O—.

In one embodiment, R₁ to R₈ each independently may be hydrogen or deuterium.

In one embodiment, L₁ and L₂ each independently may be a single bond or a substituted or unsubstituted (C₆-C₃₀)arylene, preferably a single bond or a substituted or unsubstituted (C₆-C₂₅)arylene, more preferably a single bond or a substituted or unsubstituted (C₆-C₁₃)arylene. For example, L₁ and L₂ each independently may be a single bond, a substituted or unsubstituted phenylene, a substituted or unsubstituted naphthylene, a substituted or unsubstituted biphenylene, or a substituted or unsubstituted anthracenylene.

In one embodiment, Ar₁ may be a substituted or unsubstituted (C₆-C₃₀)aryl, preferably a substituted or unsubstituted (C₆-C₂₅)aryl, more preferably a substituted or unsubstituted (C₆-C₁₈)aryl. For example, Ar₁ may be a substituted or unsubstituted phenyl, a substituted or unsubstituted biphenyl, a substituted or unsubstituted terphenyl, a substituted or unsubstituted naphthyl, a substituted or unsubstituted phenanthrenyl, or a combination thereof. For example, Ar₁ may be a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted m-terphenyl, a substituted or unsubstituted o-terphenyl, a substituted or unsubstituted p-terphenyl, or a substituted or unsubstituted phenanthrenyl, wherein the substituents of the substituted groups may be deuterium, methyl, tert-butyl, phenyl, or biphenyl.

According to one embodiment, Formula 1 may be represented by any one of the following Formulas 1-1 to 1-3.

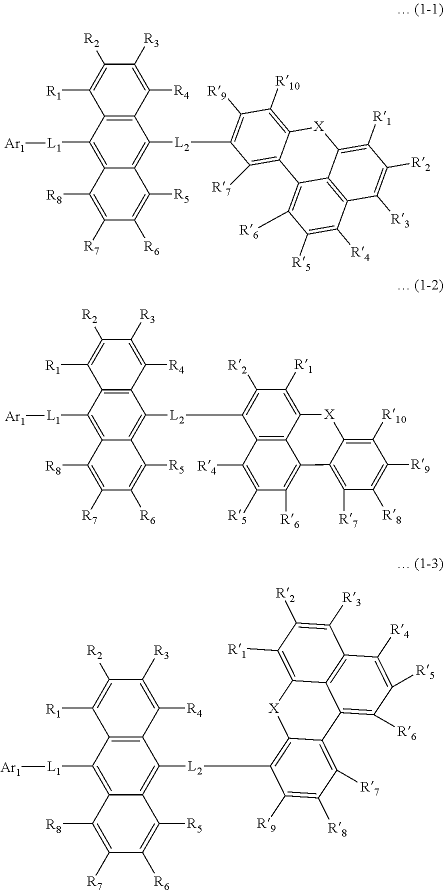

In Formulas 1-1 to 1-3,
R₁ to R₈, L₁, L₂, Ar₁, X, and R′₁ to R′₁₀ are as defined in Formula 1.

In one embodiment, R′₁ to R′₁₀ each independently may be hydrogen, deuterium, or a substituted or unsubstituted (C₆-C₃₀)aryl, preferably hydrogen, deuterium, or a substituted or unsubstituted (C₆-C₂₅)aryl, more preferably hydrogen, deuterium, or a substituted or unsubstituted (C₆-C₁₃)aryl. For example, R′₁ to R′₁₀ each independently may be hydrogen, deuterium, a substituted or unsubstituted phenyl, a substituted or unsubstituted naphthyl, or a substituted or unsubstituted biphenyl.

The first host compound represented by Formula 1 according to one embodiment may be a compound wherein R₁ to R₈ each independently represent hydrogen or deuterium, R′₁ to R′₁₀ each independently represent hydrogen, deuterium, a substituted or unsubstituted (C₆-C₁₅)aryl, or a substituted or unsubstituted (3- to 15-membered)heteroaryl.

According to one embodiment, the first host compound represented by Formula 1 may be more specifically illustrated by the following compounds, but is not limited thereto:

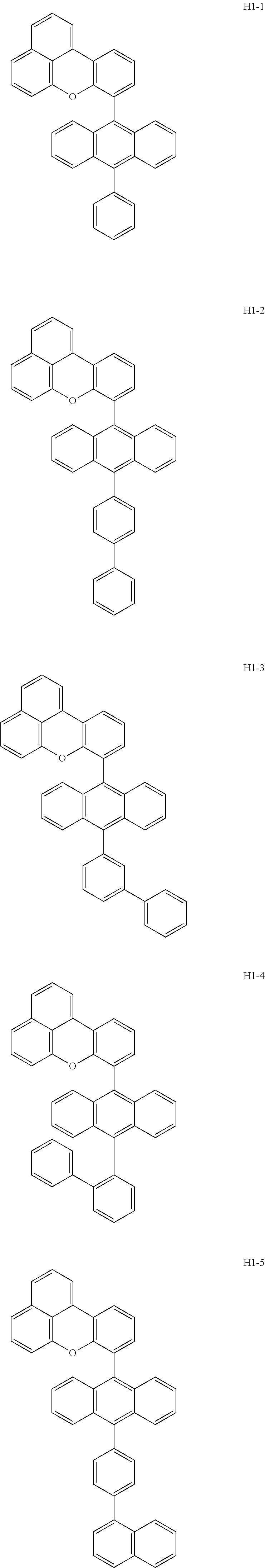 [SMILES: c1ccc(-c2c3ccccc3c(-c3cccc4c3Oc3cccc5cccc-4c35)c3ccccc23)cc1 ; c1ccc(-c2ccc(-c3c4ccccc4c(-c4cccc5c4Oc4cccc6cccc-5c46)c4ccccc34)cc2)cc1 ; c1ccc(-c2cccc(-c3c4ccccc4c(-c4cccc5c4Oc4cccc6cccc-5c46)c4ccccc34)c2)cc1 ; c1ccc(-c2ccccc2-c2c3ccccc3c(-c3cccc4c3Oc3cccc5cccc-4c35)c3ccccc23)cc1 ; c1cc(-c2c3ccccc3c(-c3ccc(-c4cccc5ccccc45)cc3)c3ccccc23)c2c(c1)-c1cccc3cccc(c13)O2]

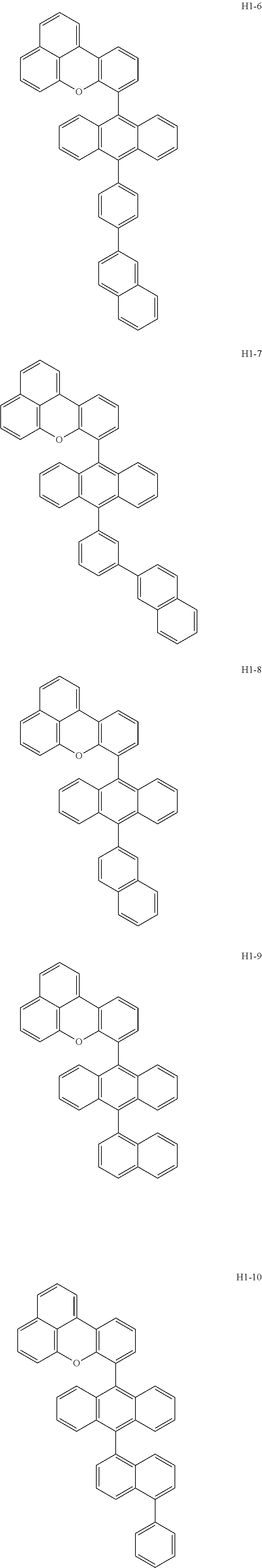 [SMILES: c1cc(-c2c3ccccc3c(-c3ccc(-c4ccc5ccccc5c4)cc3)c3ccccc23)c2c(c1)-c1cccc3cccc(c13)O2 ; c1cc(-c2ccc3ccccc3c2)cc(-c2c3ccccc3c(-c3cccc4c3Oc3cccc5cccc-4c35)c3ccccc23)c1 ; c1cc(-c2c3ccccc3c(-c3ccc4ccccc4c3)c3ccccc23)c2c(c1)-c1cccc3cccc(c13)O2 ; c1cc(-c2c3ccccc3c(-c3cccc4ccccc34)c3ccccc23)c2c(c1)-c1cccc3cccc(c13)O2 ; c1ccc(-c2cccc3c(-c4c5ccccc5c(-c5cccc6c5Oc5cccc7cccc-6c57)c5ccccc45)cccc23)cc1]

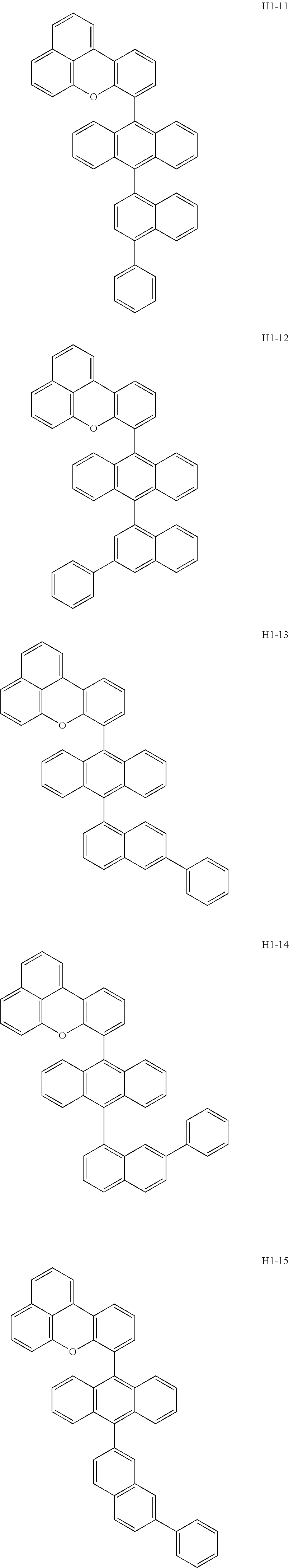 [SMILES: c1ccc(-c2ccc(-c3c4ccccc4c(-c4cccc5c4Oc4cccc6cccc-5c46)c4ccccc34)c3ccccc23)cc1 ; c1ccc(-c2cc(-c3c4ccccc4c(-c4cccc5c4Oc4cccc6cccc-5c46)c4ccccc34)c3ccccc3c2)cc1 ; c1ccc(-c2ccc3c(-c4c5ccccc5c(-c5cccc6c5Oc5cccc7cccc-6c57)c5ccccc45)cccc3c2)cc1 ; c1ccc(-c2ccc3cccc(-c4c5ccccc5c(-c5cccc6c5Oc5cccc7cccc-6c57)c5ccccc45)c3c2)cc1 ; c1ccc(-c2ccc3ccc(-c4c5ccccc5c(-c5cccc6c5Oc5cccc7cccc-6c57)c5ccccc45)cc3c2)cc1]

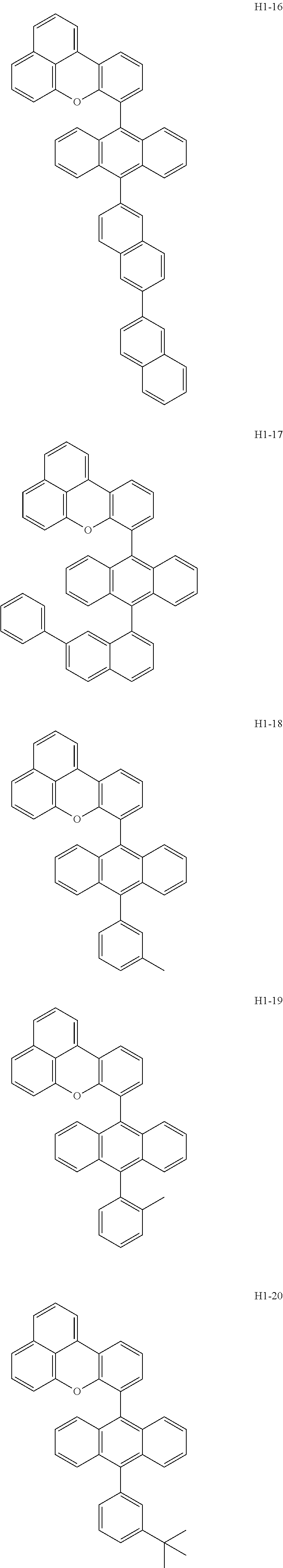 [SMILES: c1cc(-c2c3ccccc3c(-c3ccc4cc(-c5ccc6ccccc6c5)ccc4c3)c3ccccc23)c2c(c1)-c1cccc3cccc(c13)O2 ; c1ccc(-c2ccc3cccc(-c4c5ccccc5c(-c5cccc6c5Oc5cccc7cccc-6c57)c5ccccc45)c3c2)cc1 ; Cc1cccc(-c2c3ccccc3c(-c3cccc4c3Oc3cccc5cccc-4c35)c3ccccc23)c1 ; Cc1ccccc1-c1c2ccccc2c(-c2cccc3c2Oc2cccc4cccc-3c24)c2ccccc12 ; CC(C)(C)c1cccc(-c2c3ccccc3c(-c3cccc4c3Oc3cccc5cccc-4c35)c3ccccc23)c1]

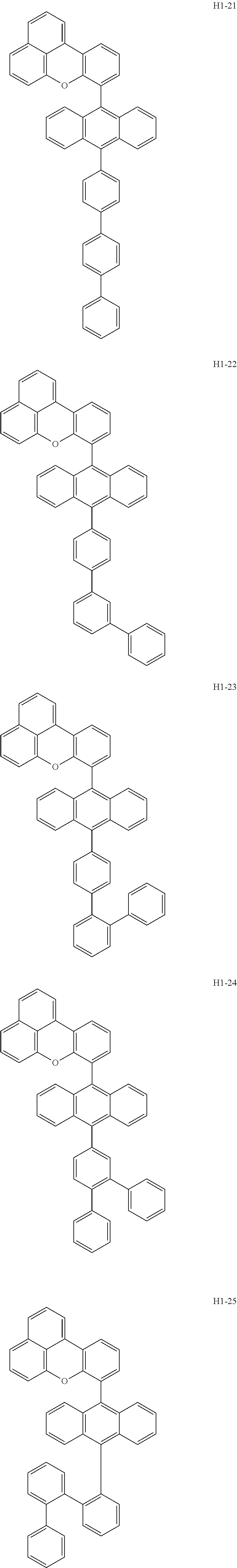 [SMILES: c1ccc(-c2ccc(-c3ccc(-c4c5ccccc5c(-c5cccc6c5Oc5cccc7cccc-6c57)c5ccccc45)cc3)cc2)cc1 ; c1ccc(-c2cccc(-c3ccc(-c4c5ccccc5c(-c5cccc6c5Oc5cccc7cccc-6c57)c5ccccc45)cc3)c2)cc1 ; c1ccc(-c2ccccc2-c2ccc(-c3c4ccccc4c(-c4cccc5c4Oc4cccc6cccc-5c46)c4ccccc34)cc2)cc1 ; c1ccc(-c2ccc(-c3c4ccccc4c(-c4cccc5c4Oc4cccc6cccc-5c46)c4ccccc34)cc2-c2ccccc2)cc1 ; c1ccc(-c2ccccc2-c2ccccc2-c2c3ccccc3c(-c3cccc4c3Oc3cccc5cccc-4c35)c3ccccc23)cc1]

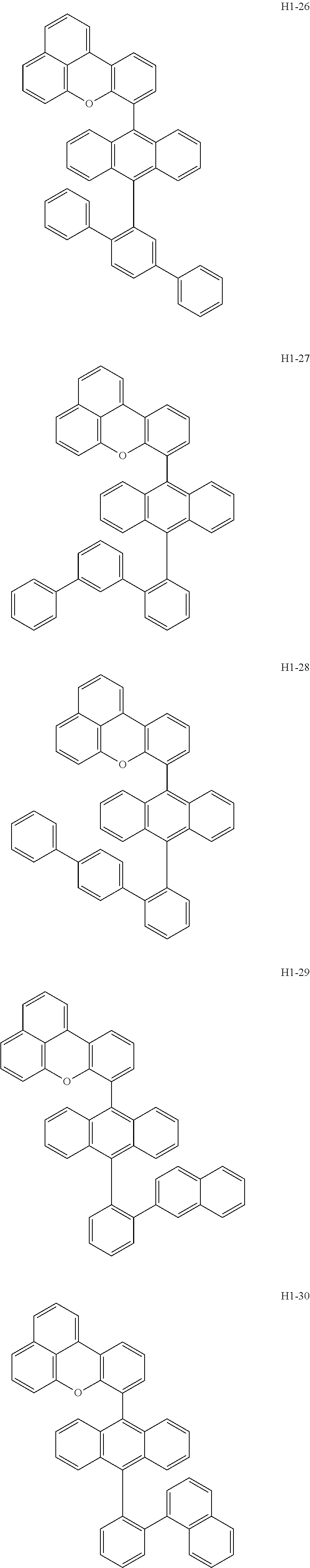 [SMILES: c1ccc(-c2ccc(-c3ccccc3)c(-c3c4ccccc4c(-c4cccc5c4Oc4cccc6cccc-5c46)c4ccccc34)c2)cc1 ; c1ccc(-c2cccc(-c3ccccc3-c3c4ccccc4c(-c4cccc5c4Oc4cccc6cccc-5c46)c4ccccc34)c2)cc1 ; c1ccc(-c2ccc(-c3ccccc3-c3c4ccccc4c(-c4cccc5c4Oc4cccc6cccc-5c46)c4ccccc34)cc2)cc1 ; c1ccc(-c2c3ccccc3c(-c3cccc4c3Oc3cccc5cccc-4c35)c3ccccc23)c(-c2ccc3ccccc3c2)c1 ; c1ccc(-c2c3ccccc3c(-c3cccc4c3Oc3cccc5cccc-4c35)c3ccccc23)c(-c2cccc3ccccc23)c1]

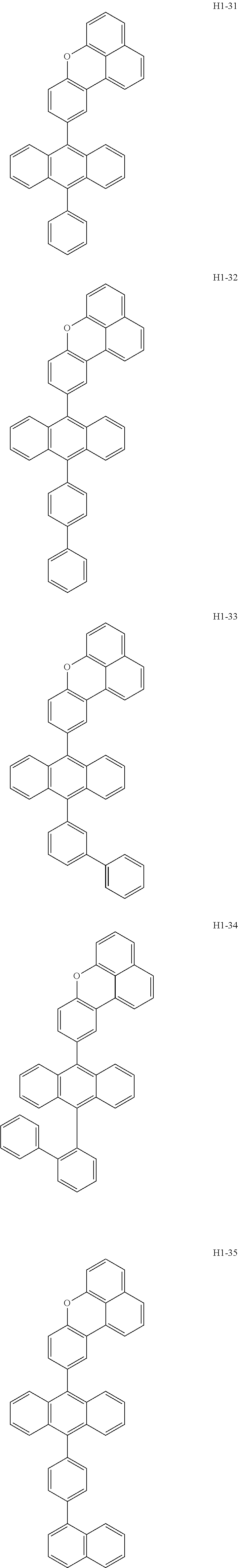 [SMILES: c1ccc(-c2c3ccccc3c(-c3ccc4c(c3)-c3cccc5cccc(c35)O4)c3ccccc23)cc1 ; c1ccc(-c2ccc(-c3c4ccccc4c(-c4ccc5c(c4)-c4cccc6cccc(c46)O5)c4ccccc34)cc2)cc1 ; c1ccc(-c2cccc(-c3c4ccccc4c(-c4ccc5c(c4)-c4cccc6cccc(c46)O5)c4ccccc34)c2)cc1 ; c1ccc(-c2ccccc2-c2c3ccccc3c(-c3ccc4c(c3)-c3cccc5cccc(c35)O4)c3ccccc23)cc1 ; c1ccc2c(-c3ccc(-c4c5ccccc5c(-c5ccc6c(c5)-c5cccc7cccc(c57)O6)c5ccccc45)cc3)cccc2c1]

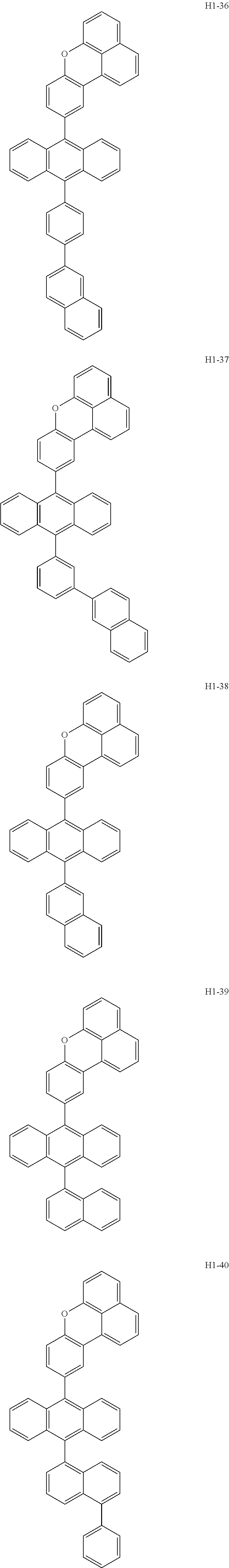

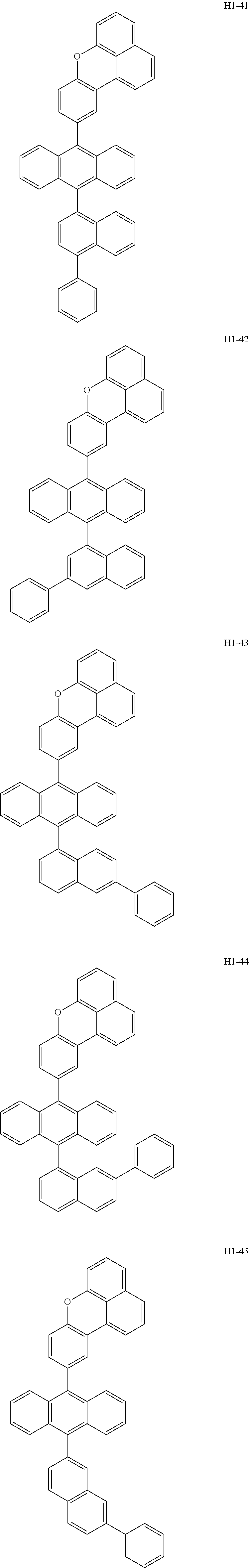 [SMILES: c1ccc(-c2ccc(-c3c4ccccc4c(-c4ccc5c(c4)-c4cccc6cccc(c46)O5)c4ccccc34)c3ccccc23)cc1 ; c1ccc(-c2cc(-c3c4ccccc4c(-c4ccc5c(c4)-c4cccc6cccc(c46)O5)c4ccccc34)c3ccccc3c2)cc1 ; c1ccc(-c2ccc3c(-c4c5ccccc5c(-c5ccc6c(c5)-c5cccc7cccc(c57)O6)c5ccccc45)cccc3c2)cc1 ; c1ccc(-c2ccc3cccc(-c4c5ccccc5c(-c5ccc6c(c5)-c5cccc7cccc(c57)O6)c5ccccc45)c3c2)cc1 ; c1ccc(-c2ccc3ccc(-c4c5ccccc5c(-c5ccc6c(c5)-c5cccc7cccc(c57)O6)c5ccccc45)cc3c2)cc1]

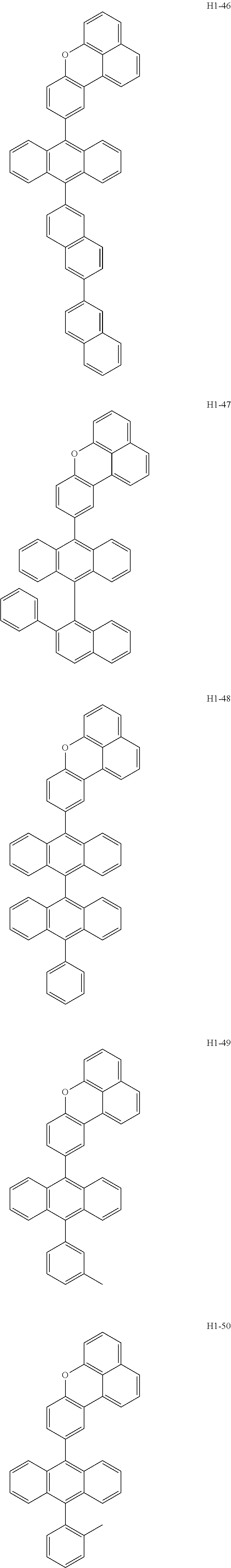 [SMILES: c1ccc2cc(-c3ccc4cc(-c5c6ccccc6c(-c6ccc7c(c6)-c6cccc8cccc(c68)O7)c6ccccc56)ccc4c3)ccc2c1 ; c1ccc(-c2ccc3ccccc3c2-c2c3ccccc3c(-c3ccc4c(c3)-c3cccc5cccc(c35)O4)c3ccccc23)cc1 ; c1ccc(-c2c3ccccc3c(-c3c4ccccc4c(-c4ccc5c(c4)-c4cccc6cccc(c46)O5)c4ccccc34)c3ccccc23)cc1 ; Cc1cccc(-c2c3ccccc3c(-c3ccc4c(c3)-c3cccc5cccc(c35)O4)c3ccccc23)c1 ; Cc1ccccc1-c1c2ccccc2c(-c2ccc3c(c2)-c2cccc4cccc(c24)O3)c2ccccc12]

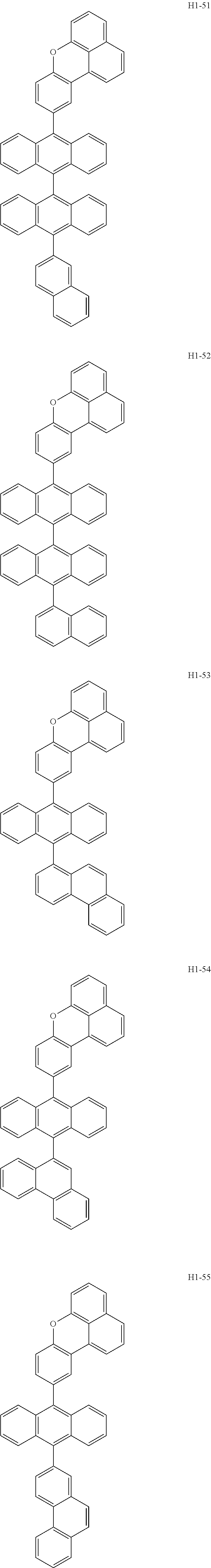 [SMILES: c1ccc2cc(-c3c4ccccc4c(-c4c5ccccc5c(-c5ccc6c(c5)-c5cccc7cccc(c57)O6)c5ccccc45)c4ccccc34)ccc2c1 ; c1ccc2c(-c3c4ccccc4c(-c4c5ccccc5c(-c5ccc6c(c5)-c5cccc7cccc(c57)O6)c5ccccc45)c4ccccc34)cccc2c1 ; c1cc2c3c(cccc3c1)-c1cc(-c3c4ccccc4c(-c4cccc5c4ccc4ccccc45)c4ccccc34)ccc1O2 ; c1cc2c3c(cccc3c1)-c1cc(-c3c4ccccc4c(-c4cc5ccccc5c5ccccc45)c4ccccc34)ccc1O2 ; c1cc2c3c(cccc3c1)-c1cc(-c3c4ccccc4c(-c4ccc5c(ccc6ccccc65)c4)c4ccccc34)ccc1O2]

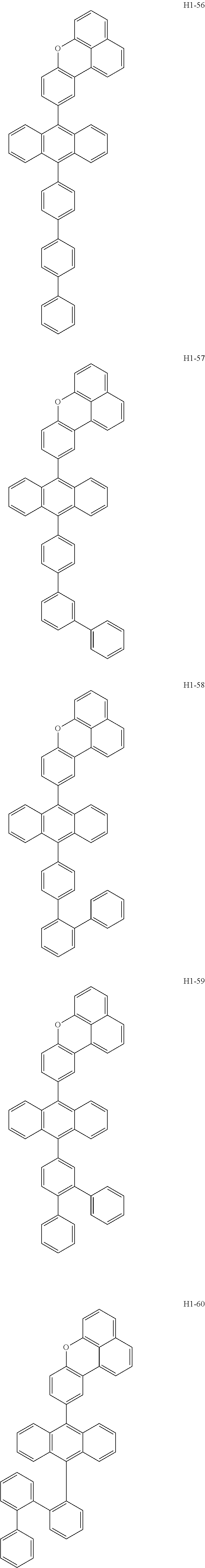 [SMILES: c1ccc(-c2ccc(-c3ccc(-c4c5ccccc5c(-c5ccc6c(c5)-c5cccc7cccc(c57)O6)c5ccccc45)cc3)cc2)cc1 ; c1ccc(-c2cccc(-c3ccc(-c4c5ccccc5c(-c5ccc6c(c5)-c5cccc7cccc(c57)O6)c5ccccc45)cc3)c2)cc1 ; c1ccc(-c2ccccc2-c2ccc(-c3c4ccccc4c(-c4ccc5c(c4)-c4cccc6cccc(c46)O5)c4ccccc34)cc2)cc1 ; c1ccc(-c2ccc(-c3c4ccccc4c(-c4ccc5c(c4)-c4cccc6cccc(c46)O5)c4ccccc34)cc2-c2ccccc2)cc1 ; c1ccc(-c2ccccc2-c2ccccc2-c2c3ccccc3c(-c3ccc4c(c3)-c3cccc5cccc(c35)O4)c3ccccc23)cc1]

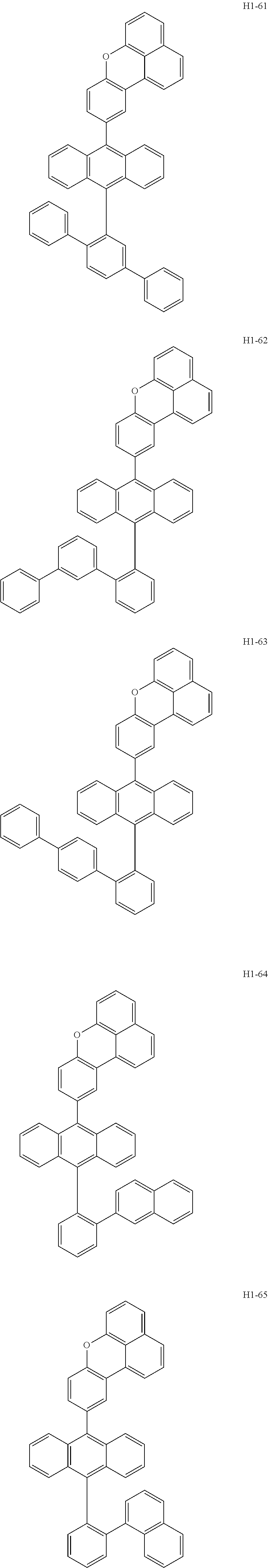 [SMILES: c1ccc(-c2ccc(-c3ccccc3)c(-c3c4ccccc4c(-c4ccc5c(c4)-c4cccc6cccc(c46)O5)c4ccccc34)c2)cc1 ; c1ccc(-c2cccc(-c3ccccc3-c3c4ccccc4c(-c4ccc5c(c4)-c4cccc6cccc(c46)O5)c4ccccc34)c2)cc1 ; c1ccc(-c2ccc(-c3ccccc3-c3c4ccccc4c(-c4ccc5c(c4)-c4cccc6cccc(c46)O5)c4ccccc34)cc2)cc1 ; c1ccc(-c2c3ccccc3c(-c3ccc4c(c3)-c3cccc5cccc(c35)O4)c3ccccc23)c(-c2ccc3ccccc3c2)c1 ; c1ccc(-c2c3ccccc3c(-c3ccc4c(c3)-c3cccc5cccc(c35)O4)c3ccccc23)c(-c2cccc3ccccc23)c1]

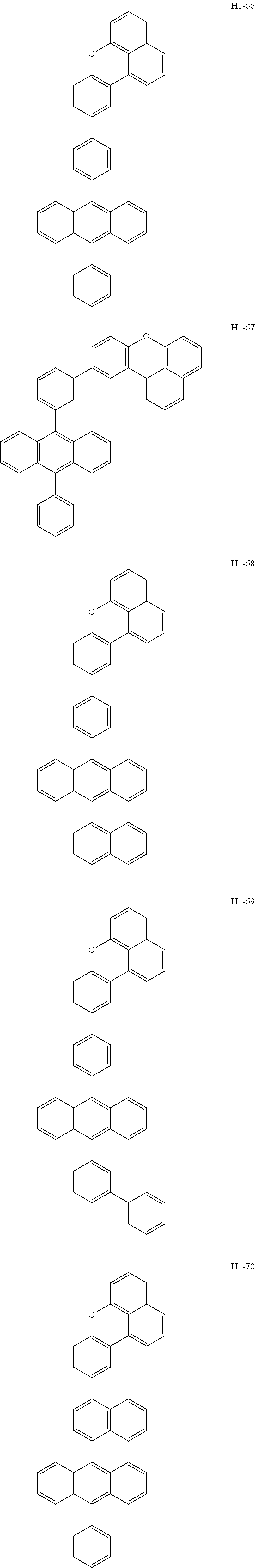 [SMILES: c1ccc(-c2c3ccccc3c(-c3ccc(-c4ccc5c(c4)-c4cccc6cccc(c46)O5)cc3)c3ccccc23)cc1 ; c1ccc(-c2c3ccccc3c(-c3cccc(-c4ccc5c(c4)-c4cccc6cccc(c46)O5)c3)c3ccccc23)cc1 ; c1ccc2c(-c3c4ccccc4c(-c4ccc(-c5ccc6c(c5)-c5cccc7cccc(c57)O6)cc4)c4ccccc34)cccc2c1 ; c1ccc(-c2cccc(-c3c4ccccc4c(-c4ccc(-c5ccc6c(c5)-c5cccc7cccc(c57)O6)cc4)c4ccccc34)c2)cc1 ; c1ccc(-c2c3ccccc3c(-c3ccc(-c4ccc5c(c4)-c4cccc6cccc(c46)O5)c4ccccc34)c3ccccc23)cc1]

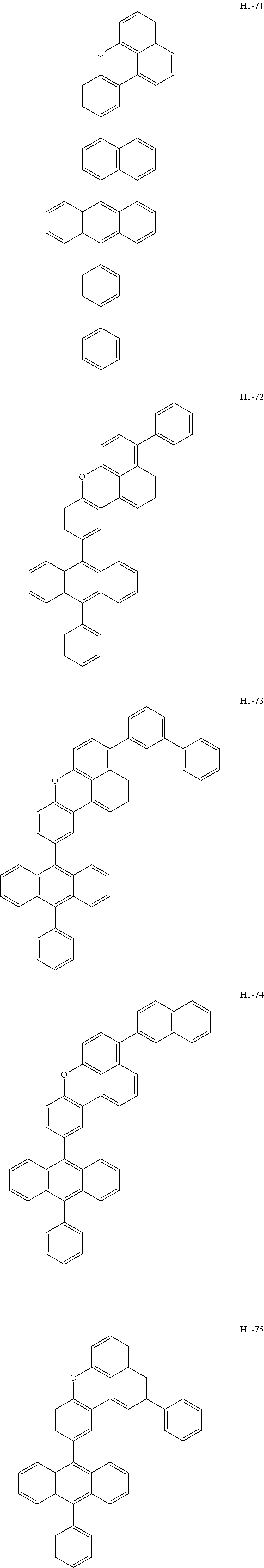 [SMILES: c1ccc(-c2ccc(-c3c4ccccc4c(-c4ccc(-c5ccc6c(c5)-c5cccc7cccc(c57)O6)c5ccccc45)c4ccccc34)cc2)cc1 ; c1ccc(-c2c3ccccc3c(-c3ccc4c(c3)-c3cccc5c(-c6ccccc6)ccc(c35)O4)c3ccccc23)cc1 ; c1ccc(-c2cccc(-c3ccc4c5c(cccc35)-c3cc(-c5c6ccccc6c(-c6ccccc6)c6ccccc56)ccc3O4)c2)cc1 ; c1ccc(-c2c3ccccc3c(-c3ccc4c(c3)-c3cccc5c(-c6ccc7ccccc7c6)ccc(c35)O4)c3ccccc23)cc1 ; c1ccc(-c2cc3c4c(cccc4c2)Oc2ccc(-c4c5ccccc5c(-c5ccccc5)c5ccccc45)cc2-3)cc1]

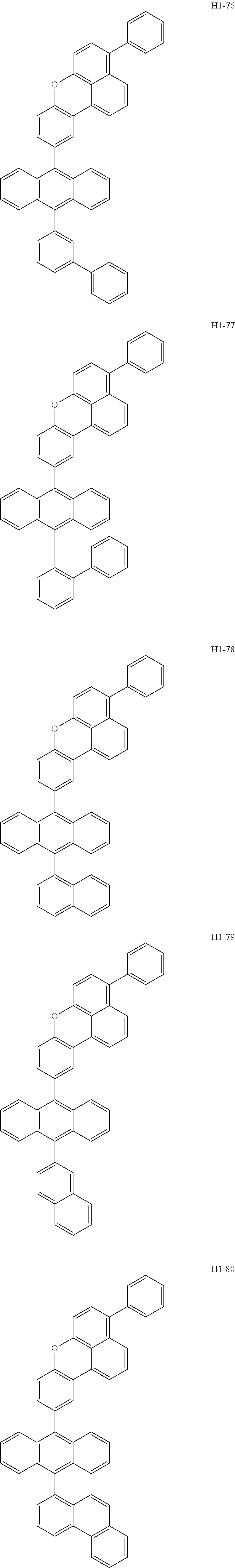 [SMILES: c1ccc(-c2cccc(-c3c4ccccc4c(-c4ccc5c(c4)-c4cccc6c(-c7ccccc7)ccc(c46)O5)c4ccccc34)c2)cc1 ; c1ccc(-c2ccccc2-c2c3ccccc3c(-c3ccc4c(c3)-c3cccc5c(-c6ccccc6)ccc(c35)O4)c3ccccc23)cc1 ; c1ccc(-c2ccc3c4c(cccc24)-c2cc(-c4c5ccccc5c(-c5cccc6ccccc56)c5ccccc45)ccc2O3)cc1 ; c1ccc(-c2ccc3c4c(cccc24)-c2cc(-c4c5ccccc5c(-c5ccc6ccccc6c5)c5ccccc45)ccc2O3)cc1 ; c1ccc(-c2ccc3c4c(cccc24)-c2cc(-c4c5ccccc5c(-c5cccc6c5ccc5ccccc56)c5ccccc45)ccc2O3)cc1]

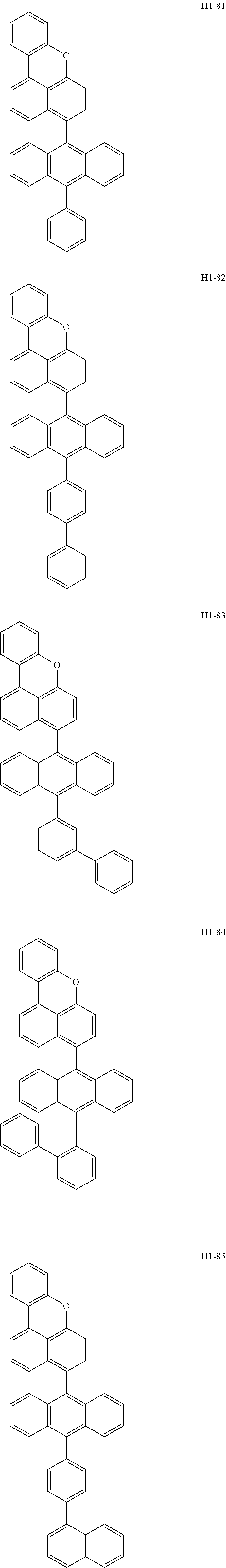 [SMILES: c1ccc(-c2c3ccccc3c(-c3ccc4c5c(cccc35)-c3ccccc3O4)c3ccccc23)cc1 ; c1ccc(-c2ccc(-c3c4ccccc4c(-c4ccc5c6c(cccc46)-c4ccccc4O5)c4ccccc34)cc2)cc1 ; c1ccc(-c2cccc(-c3c4ccccc4c(-c4ccc5c6c(cccc46)-c4ccccc4O5)c4ccccc34)c2)cc1 ; c1ccc(-c2ccccc2-c2c3ccccc3c(-c3ccc4c5c(cccc35)-c3ccccc3O4)c3ccccc23)cc1 ; c1ccc2c(c1)Oc1ccc(-c3c4ccccc4c(-c4ccc(-c5cccc6ccccc56)cc4)c4ccccc34)c3cccc-2c13]

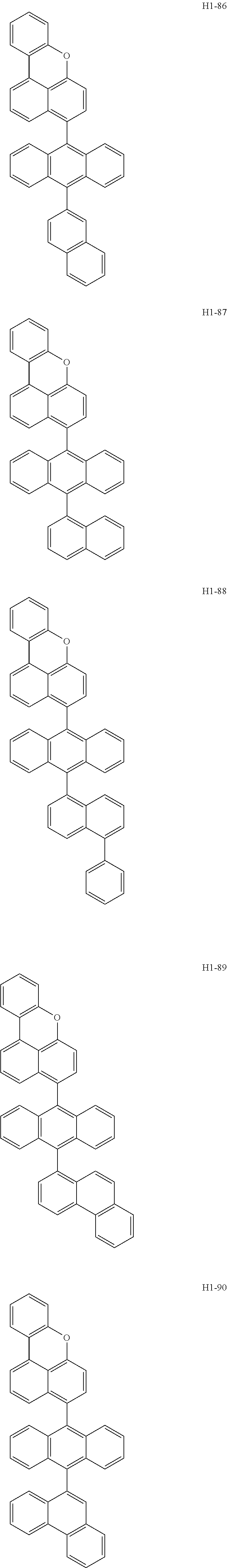 [SMILES: c1ccc2c(c1)Oc1ccc(-c3c4ccccc4c(-c4ccc5ccccc5c4)c4ccccc34)c3cccc-2c13 ; c1ccc2c(c1)Oc1ccc(-c3c4ccccc4c(-c4cccc5ccccc45)c4ccccc34)c3cccc-2c13 ; c1ccc(-c2cccc3c(-c4c5ccccc5c(-c5ccc6c7c(cccc57)-c5ccccc5O6)c5ccccc45)cccc23)cc1 ; c1ccc2c(c1)Oc1ccc(-c3c4ccccc4c(-c4cccc5c4ccc4ccccc45)c4ccccc34)c3cccc-2c13 ; c1ccc2c(c1)Oc1ccc(-c3c4ccccc4c(-c4cc5ccccc5c5ccccc45)c4ccccc34)c3cccc-2c13]

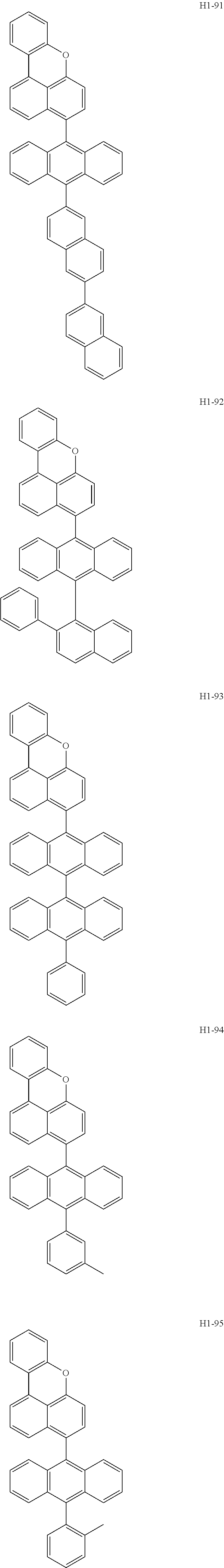 [SMILES: c1ccc2c(c1)Oc1ccc(-c3c4ccccc4c(-c4ccc5cc(-c6ccc7ccccc7c6)ccc5c4)c4ccccc34)c3cccc-2c13 ; c1ccc(-c2ccc3ccccc3c2-c2c3ccccc3c(-c3ccc4c5c(cccc35)-c3ccccc3O4)c3ccccc23)cc1 ; c1ccc(-c2c3ccccc3c(-c3c4ccccc4c(-c4ccc5c6c(cccc46)-c4ccccc4O5)c4ccccc34)c3ccccc23)cc1 ; Cc1cccc(-c2c3ccccc3c(-c3ccc4c5c(cccc35)-c3ccccc3O4)c3ccccc23)c1 ; Cc1ccccc1-c1c2ccccc2c(-c2ccc3c4c(cccc24)-c2ccccc2O3)c2ccccc12]

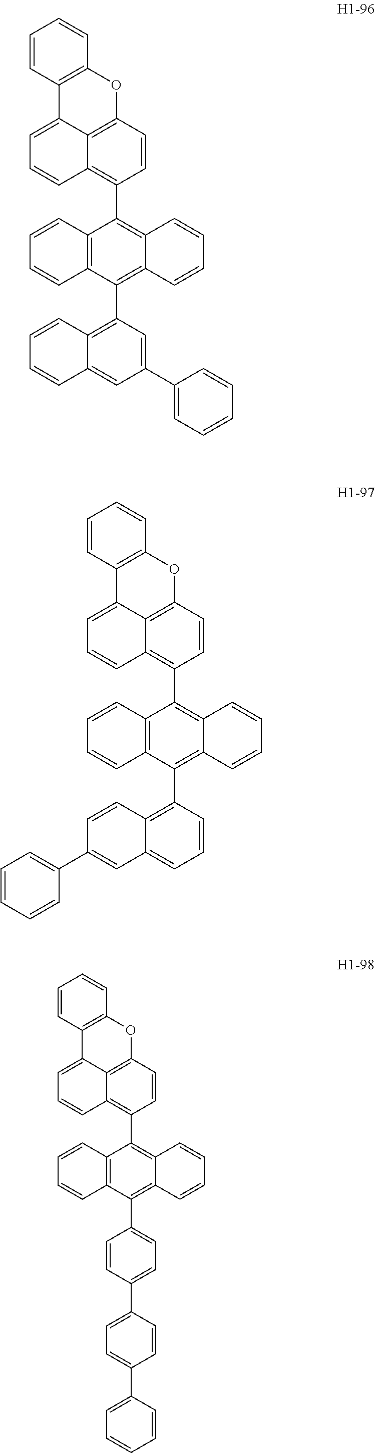 [SMILES: c1ccc(-c2cc(-c3c4ccccc4c(-c4ccc5c6c(cccc46)-c4ccccc4O5)c4ccccc34)c3ccccc3c2)cc1 ; c1ccc(-c2ccc3c(-c4c5ccccc5c(-c5ccc6c7c(cccc57)-c5ccccc5O6)c5ccccc45)cccc3c2)cc1 ; c1ccc(-c2ccc(-c3ccc(-c4c5ccccc5c(-c5ccc6c7c(cccc57)-c5ccccc5O6)c5ccccc45)cc3)cc2)cc1]

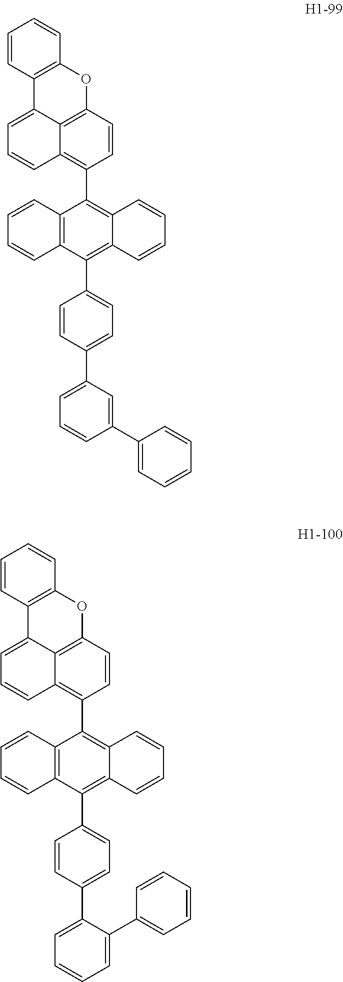 [SMILES: c1ccc(-c2cccc(-c3ccc(-c4c5ccccc5c(-c5ccc6c7c(cccc57)-c5ccccc5O6)c5ccccc45)cc3)c2)cc1 ; c1ccc(-c2ccccc2-c2ccc(-c3c4ccccc4c(-c4ccc5c6c(cccc46)-c4ccccc4O5)c4ccccc34)cc2)cc1]

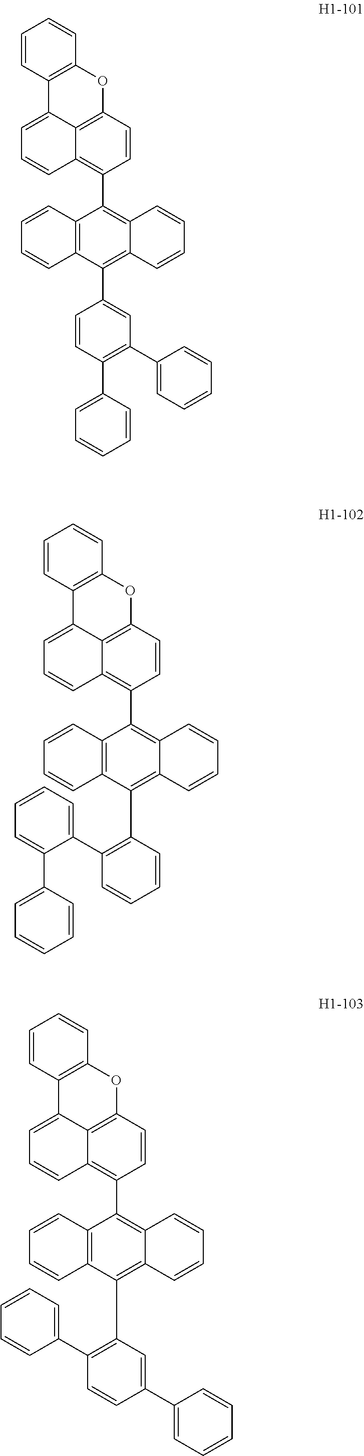 [SMILES: c1ccc(-c2ccc(-c3c4ccccc4c(-c4ccc5c6c(cccc46)-c4ccccc4O5)c4ccccc34)cc2-c2ccccc2)cc1 ; c1ccc(-c2ccccc2-c2ccccc2-c2c3ccccc3c(-c3ccc4c5c(cccc35)-c3ccccc3O4)c3ccccc23)cc1 ; c1ccc(-c2ccc(-c3ccccc3)c(-c3c4ccccc4c(-c4ccc5c6c(cccc46)-c4ccccc4O5)c4ccccc34)c2)cc1]

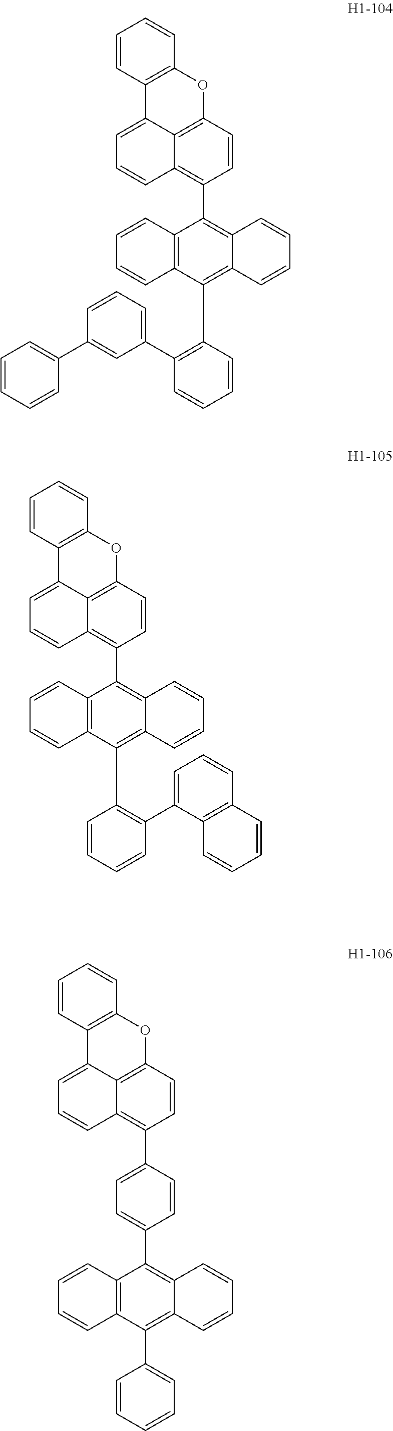 [SMILES: c1ccc(-c2cccc(-c3ccccc3-c3c4ccccc4c(-c4ccc5c6c(cccc46)-c4ccccc4O5)c4ccccc34)c2)cc1 ; c1ccc2c(c1)Oc1ccc(-c3c4ccccc4c(-c4ccccc4-c4cccc5ccccc45)c4ccccc34)c3cccc-2c13 ; c1ccc(-c2c3ccccc3c(-c3ccc(-c4ccc5c6c(cccc46)-c4ccccc4O5)cc3)c3ccccc23)cc1]

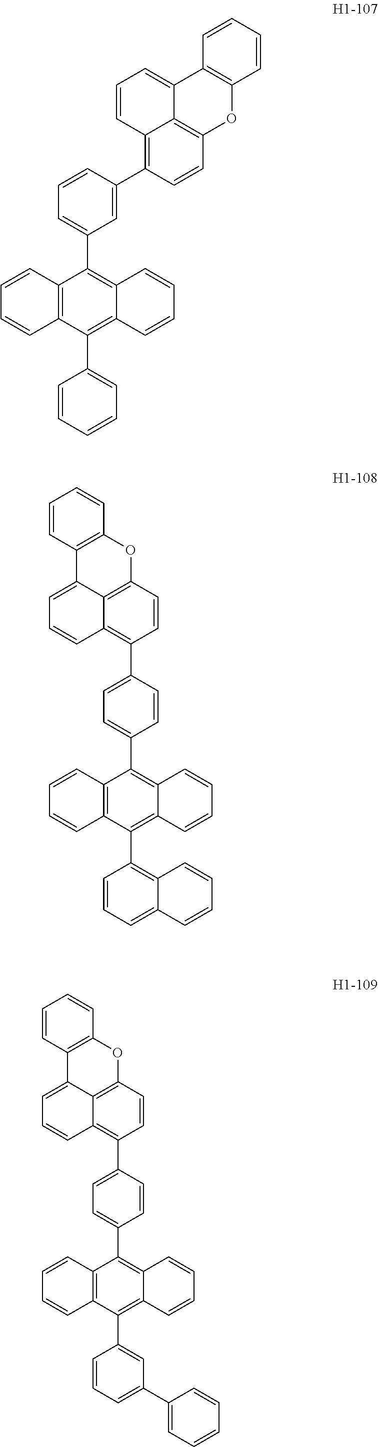 [SMILES: c1ccc(-c2c3ccccc3c(-c3cccc(-c4ccc5c6c(cccc46)-c4ccccc4O5)c3)c3ccccc23)cc1 ; c1ccc2c(c1)Oc1ccc(-c3ccc(-c4c5ccccc5c(-c5cccc6ccccc56)c5ccccc45)cc3)c3cccc-2c13 ; c1ccc(-c2cccc(-c3c4ccccc4c(-c4ccc(-c5ccc6c7c(cccc57)-c5ccccc5O6)cc4)c4ccccc34)c2)cc1]

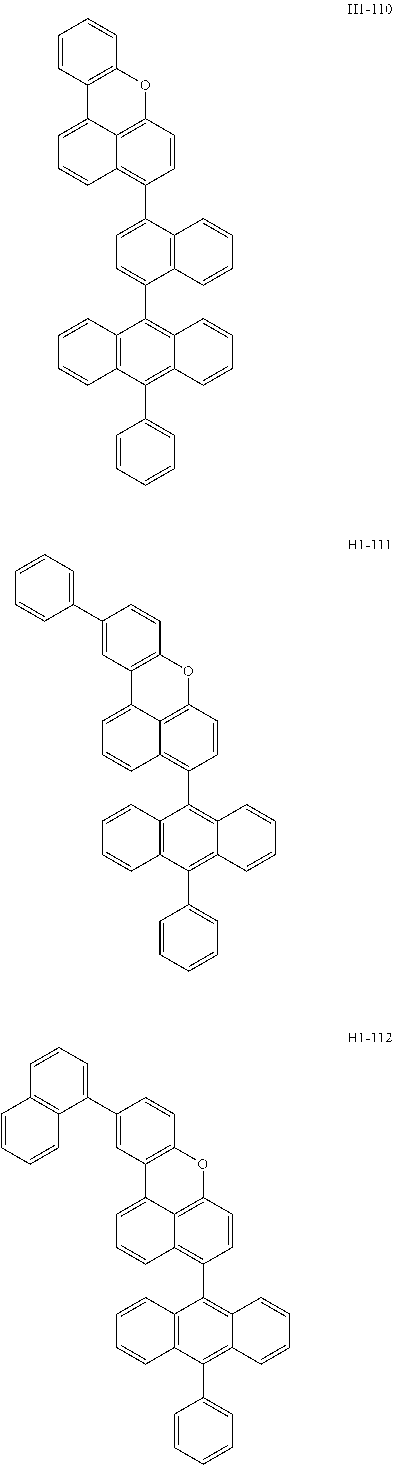 [SMILES: c1ccc(-c2c3ccccc3c(-c3ccc(-c4ccc5c6c(cccc46)-c4ccccc4O5)c4ccccc34)c3ccccc23)cc1 ; c1ccc(-c2ccc3c(c2)-c2cccc4c(-c5c6ccccc6c(-c6ccccc6)c6ccccc56)ccc(c24)O3)cc1 ; c1ccc(-c2c3ccccc3c(-c3ccc4c5c(cccc35)-c3cc(-c5cccc6ccccc56)ccc3O4)c3ccccc23)cc1]

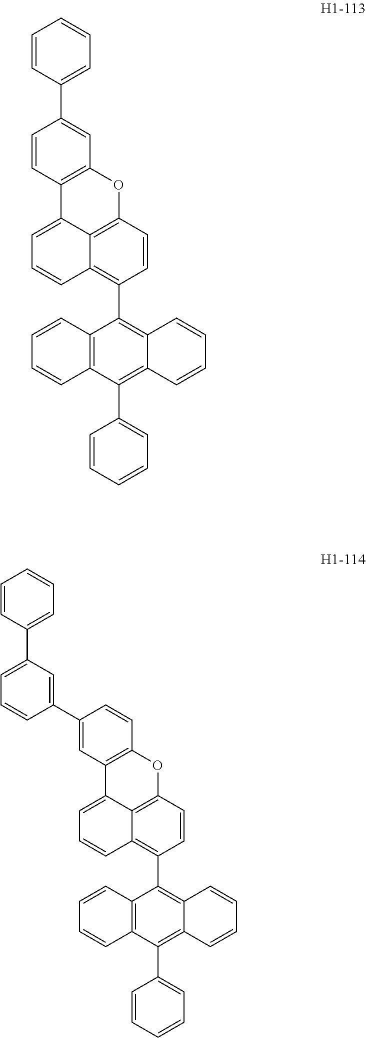 [SMILES: c1ccc(-c2ccc3c(c2)Oc2ccc(-c4c5ccccc5c(-c5ccccc5)c5ccccc45)c4cccc-3c24)cc1 ; c1ccc(-c2cccc(-c3ccc4c(c3)-c3cccc5c(-c6c7ccccc7c(-c7ccccc7)c7ccccc67)ccc(c35)O4)c2)cc1]

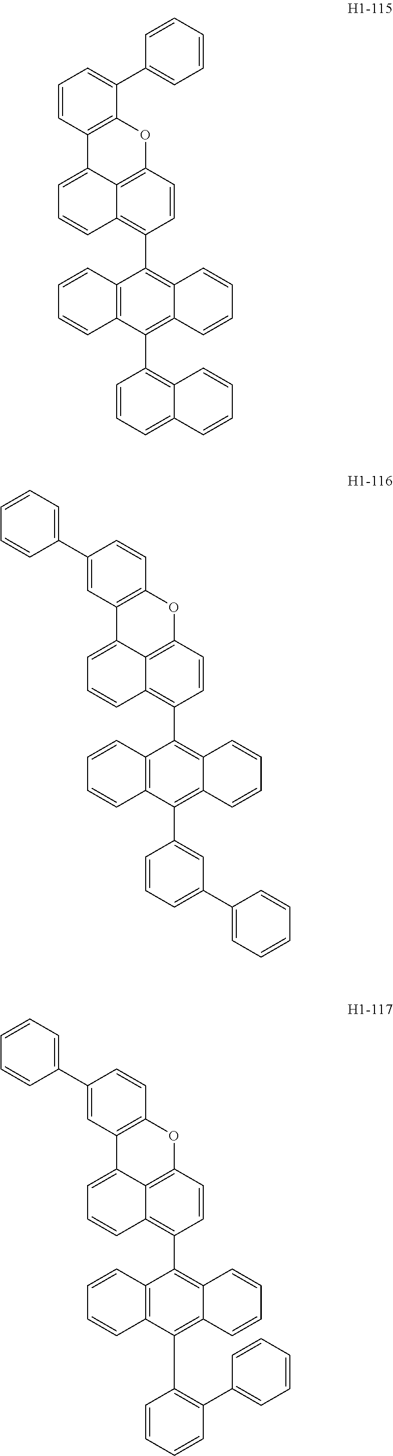 [SMILES: c1ccc(-c2cccc3c2Oc2ccc(-c4c5ccccc5c(-c5cccc6ccccc56)c5ccccc45)c4cccc-3c24)cc1 ; c1ccc(-c2cccc(-c3c4ccccc4c(-c4ccc5c6c(cccc46)-c4cc(-c6ccccc6)ccc4O5)c4ccccc34)c2)cc1 ; c1ccc(-c2ccc3c(c2)-c2cccc4c(-c5c6ccccc6c(-c6ccccc6-c6ccccc6)c6ccccc56)ccc(c24)O3)cc1]

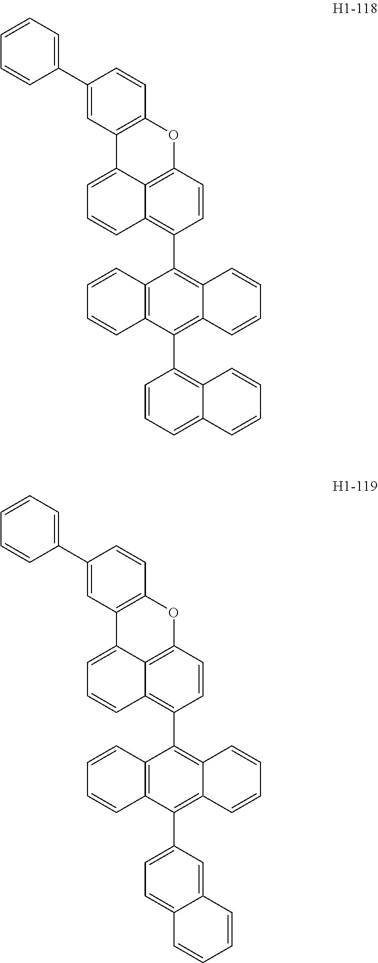 [SMILES: c1ccc(-c2ccc3c(c2)-c2cccc4c(-c5c6ccccc6c(-c6cccc7ccccc67)c6ccccc56)ccc(c24)O3)cc1 ; c1ccc(-c2ccc3c(c2)-c2cccc4c(-c5c6ccccc6c(-c6ccc7ccccc7c6)c6ccccc56)ccc(c24)O3)cc1]

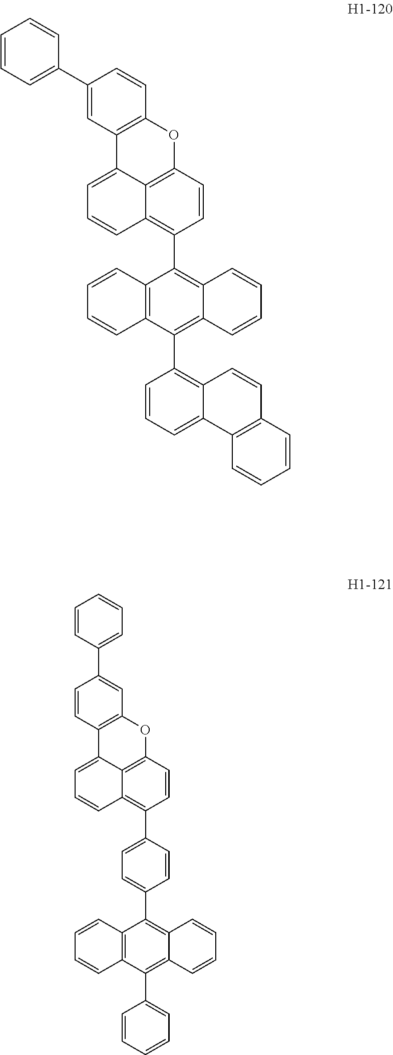 [SMILES: c1ccc(-c2ccc3c(c2)-c2cccc4c(-c5c6ccccc6c(-c6cccc7c6ccc6ccccc67)c6ccccc56)ccc(c24)O3)cc1 ; c1ccc(-c2ccc3c(c2)Oc2ccc(-c4ccc(-c5c6ccccc6c(-c6ccccc6)c6ccccc56)cc4)c4cccc-3c24)cc1]

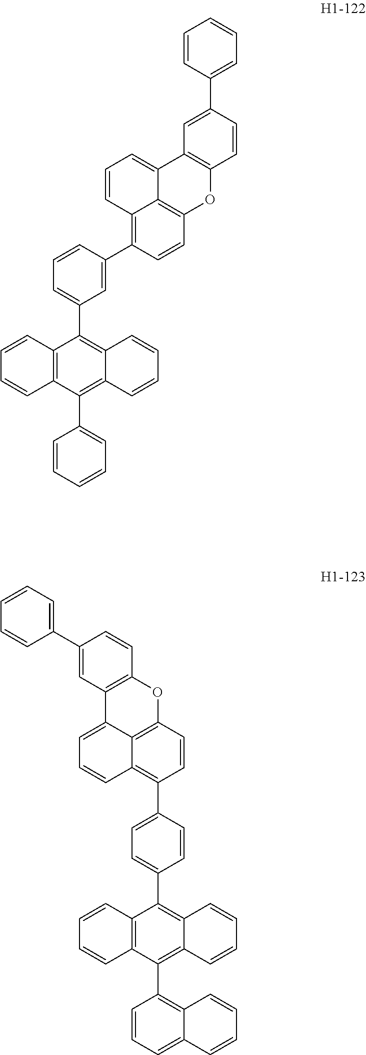 [SMILES: c1ccc(-c2ccc3c(c2)-c2cccc4c(-c5cccc(-c6c7ccccc7c(-c7ccccc7)c7ccccc67)c5)ccc(c24)O3)cc1 ; c1ccc(-c2ccc3c(c2)-c2cccc4c(-c5ccc(-c6c7ccccc7c(-c7cccc8ccccc78)c7ccccc67)cc5)ccc(c24)O3)cc1]

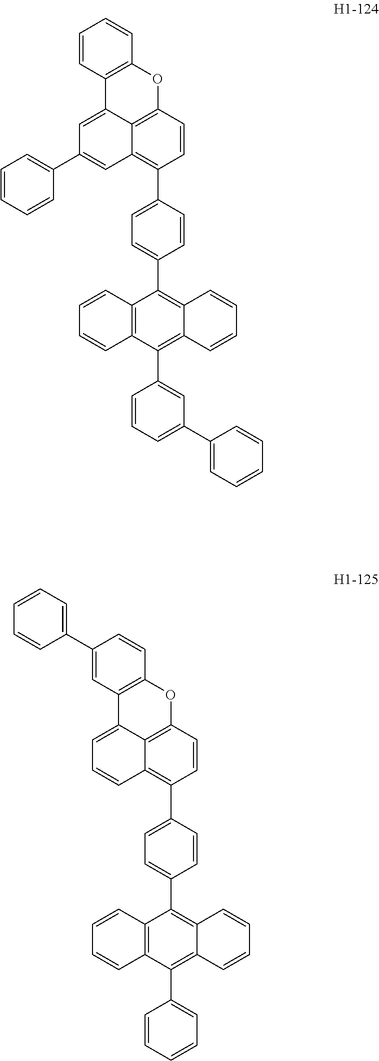 [SMILES: c1ccc(-c2cccc(-c3c4ccccc4c(-c4ccc(-c5ccc6c7c(cc(-c8ccccc8)cc57)-c5ccccc5O6)cc4)c4ccccc34)c2)cc1 ; c1ccc(-c2ccc3c(c2)-c2cccc4c(-c5ccc(-c6c7ccccc7c(-c7ccccc7)c7ccccc67)cc5)ccc(c24)O3)cc1]

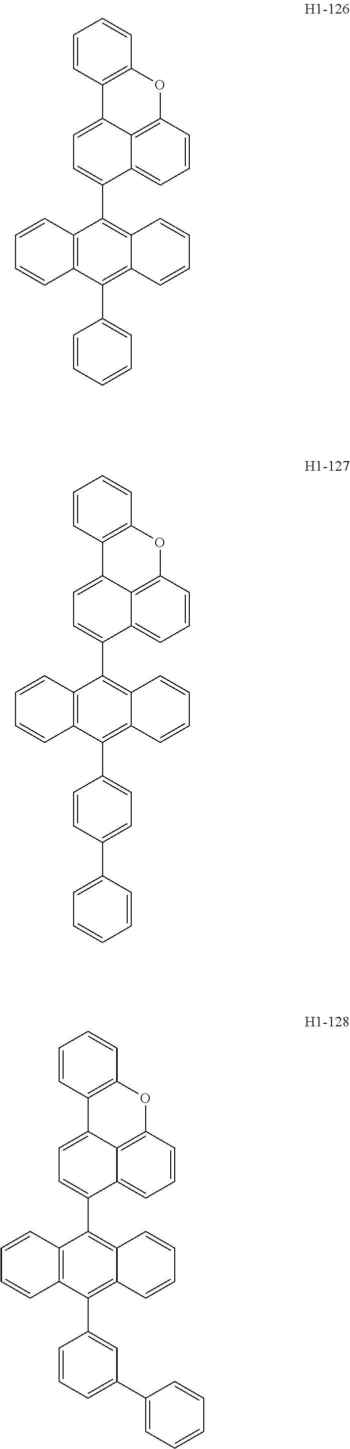 [SMILES: c1ccc(-c2c3ccccc3c(-c3ccc4c5c(cccc35)Oc3ccccc3-4)c3ccccc23)cc1 ; c1ccc(-c2ccc(-c3c4ccccc4c(-c4ccc5c6c(cccc46)Oc4ccccc4-5)c4ccccc34)cc2)cc1 ; c1ccc(-c2cccc(-c3c4ccccc4c(-c4ccc5c6c(cccc46)Oc4ccccc4-5)c4ccccc34)c2)cc1]

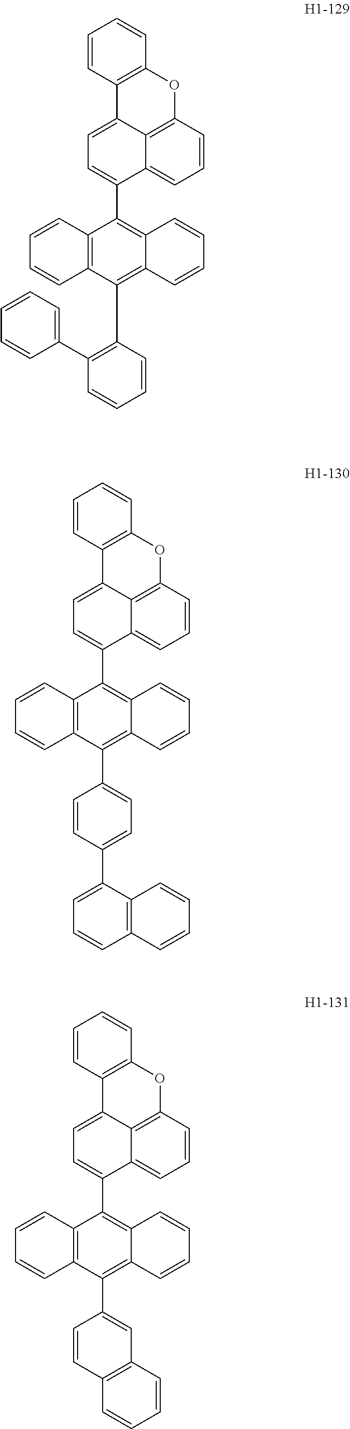 [SMILES: c1ccc(-c2ccccc2-c2c3ccccc3c(-c3ccc4c5c(cccc35)Oc3ccccc3-4)c3ccccc23)cc1 ; c1ccc2c(c1)Oc1cccc3c(-c4c5ccccc5c(-c5ccc(-c6cccc7ccccc67)cc5)c5ccccc45)ccc-2c13 ; c1ccc2c(c1)Oc1cccc3c(-c4c5ccccc5c(-c5ccc6ccccc6c5)c5ccccc45)ccc-2c13]

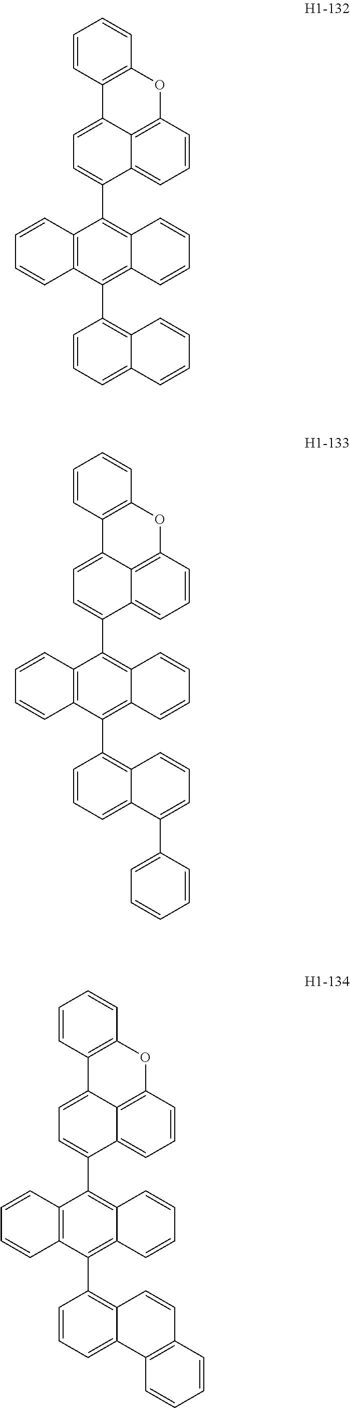 [SMILES: c1ccc2c(c1)Oc1cccc3c(-c4c5ccccc5c(-c5cccc6ccccc56)c5ccccc45)ccc-2c13 ; c1ccc(-c2cccc3c(-c4c5ccccc5c(-c5ccc6c7c(cccc57)Oc5ccccc5-6)c5ccccc45)cccc23)cc1 ; c1ccc2c(c1)Oc1cccc3c(-c4c5ccccc5c(-c5cccc6c5ccc5ccccc56)c5ccccc45)ccc-2c13]

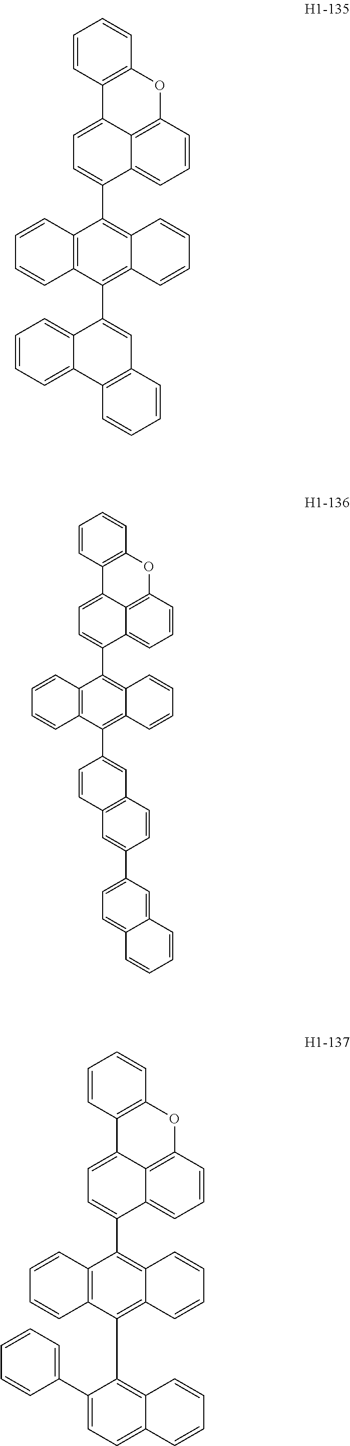 [SMILES: c1ccc2c(c1)Oc1cccc3c(-c4c5ccccc5c(-c5cc6ccccc6c6ccccc56)c5ccccc45)ccc-2c13 ; c1ccc2c(c1)Oc1cccc3c(-c4c5ccccc5c(-c5ccc6cc(-c7ccc8ccccc8c7)ccc6c5)c5ccccc45)ccc-2c13 ; c1ccc(-c2ccc3ccccc3c2-c2c3ccccc3c(-c3ccc4c5c(cccc35)Oc3ccccc3-4)c3ccccc23)cc1]

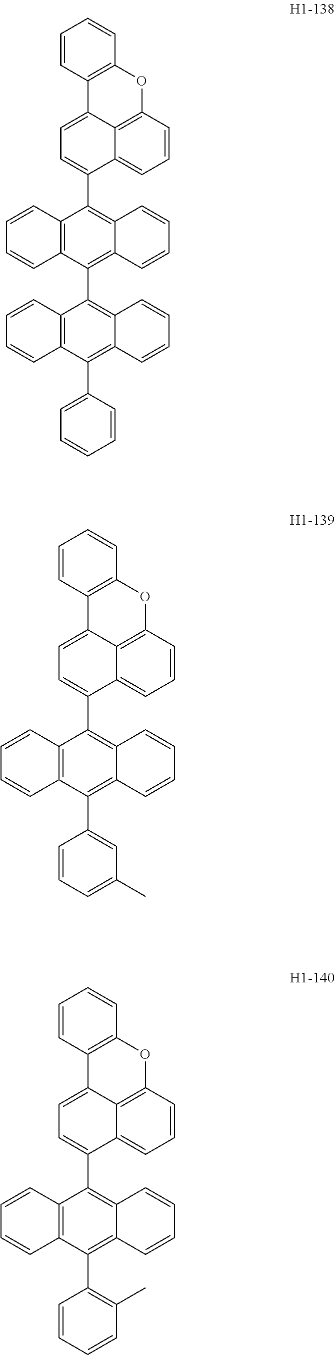 [SMILES: c1ccc(-c2c3ccccc3c(-c3c4ccccc4c(-c4ccc5c6c(cccc46)Oc4ccccc4-5)c4ccccc34)c3ccccc23)cc1 ; Cc1cccc(-c2c3ccccc3c(-c3ccc4c5c(cccc35)Oc3ccccc3-4)c3ccccc23)c1 ; Cc1ccccc1-c1c2ccccc2c(-c2ccc3c4c(cccc24)Oc2ccccc2-3)c2ccccc12]

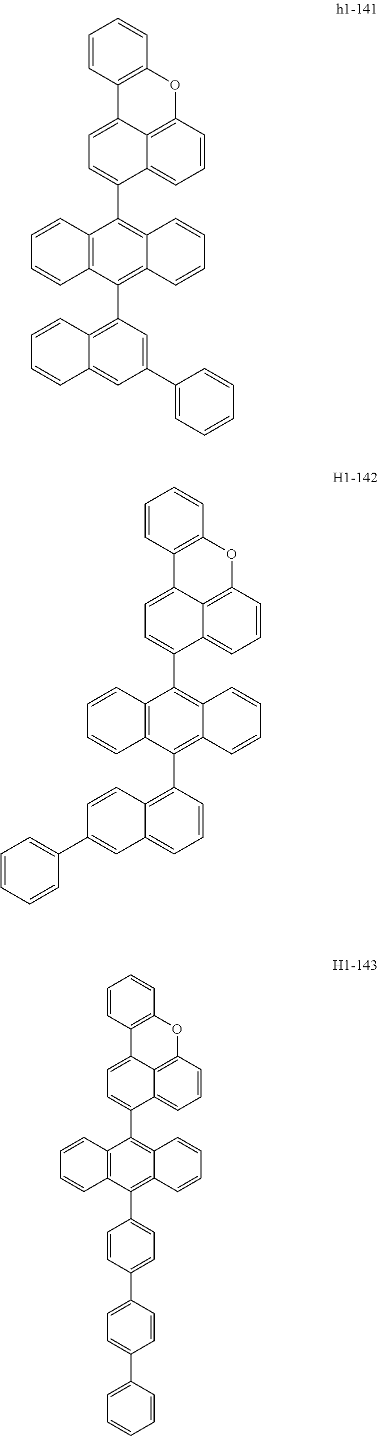 [SMILES: c1ccc(-c2cc(-c3c4ccccc4c(-c4ccc5c6c(cccc46)Oc4ccccc4-5)c4ccccc34)c3ccccc3c2)cc1 ; c1ccc(-c2ccc3c(-c4c5ccccc5c(-c5ccc6c7c(cccc57)Oc5ccccc5-6)c5ccccc45)cccc3c2)cc1 ; c1ccc(-c2ccc(-c3ccc(-c4c5ccccc5c(-c5ccc6c7c(cccc57)Oc5ccccc5-6)c5ccccc45)cc3)cc2)cc1]

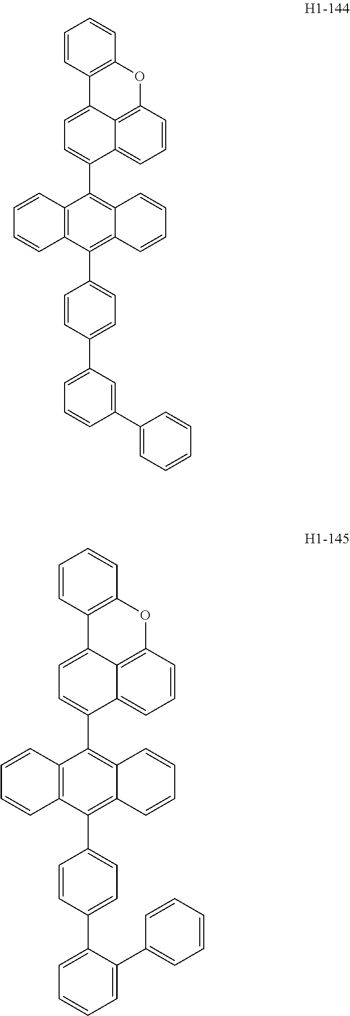 [SMILES: c1ccc(-c2cccc(-c3ccc(-c4c5ccccc5c(-c5ccc6c7c(cccc57)Oc5ccccc5-6)c5ccccc45)cc3)c2)cc1 ; c1ccc(-c2ccccc2-c2ccc(-c3c4ccccc4c(-c4ccc5c6c(cccc46)Oc4ccccc4-5)c4ccccc34)cc2)cc1]

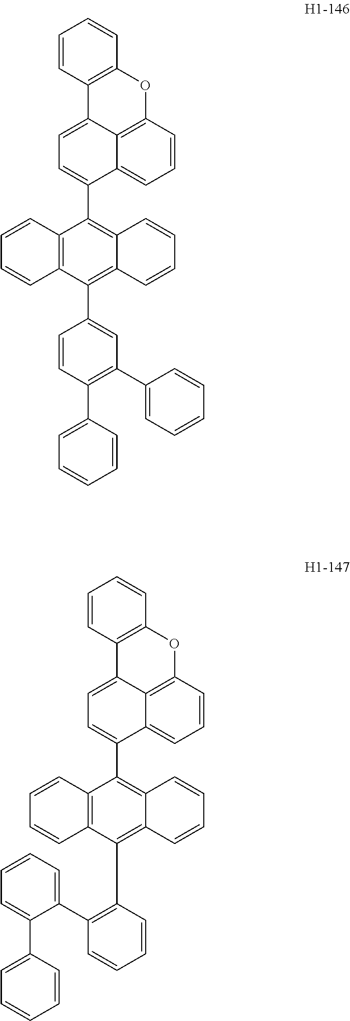 [SMILES: c1ccc(-c2ccc(-c3c4ccccc4c(-c4ccc5c6c(cccc46)Oc4ccccc4-5)c4ccccc34)cc2-c2ccccc2)cc1 ; c1ccc(-c2ccccc2-c2ccccc2-c2c3ccccc3c(-c3ccc4c5c(cccc35)Oc3ccccc3-4)c3ccccc23)cc1]

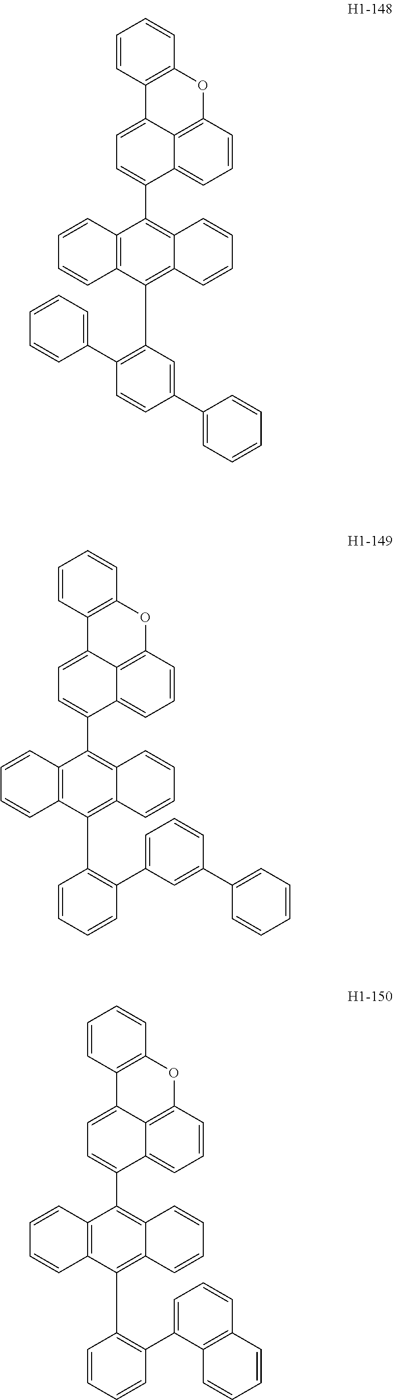 [SMILES: c1ccc(-c2ccc(-c3ccccc3)c(-c3c4ccccc4c(-c4ccc5c6c(cccc46)Oc4ccccc4-5)c4ccccc34)c2)cc1 ; c1ccc(-c2cccc(-c3ccccc3-c3c4ccccc4c(-c4ccc5c6c(cccc46)Oc4ccccc4-5)c4ccccc34)c2)cc1 ; c1ccc2c(c1)Oc1cccc3c(-c4c5ccccc5c(-c5ccccc5-c5cccc6ccccc56)c5ccccc45)ccc-2c13]

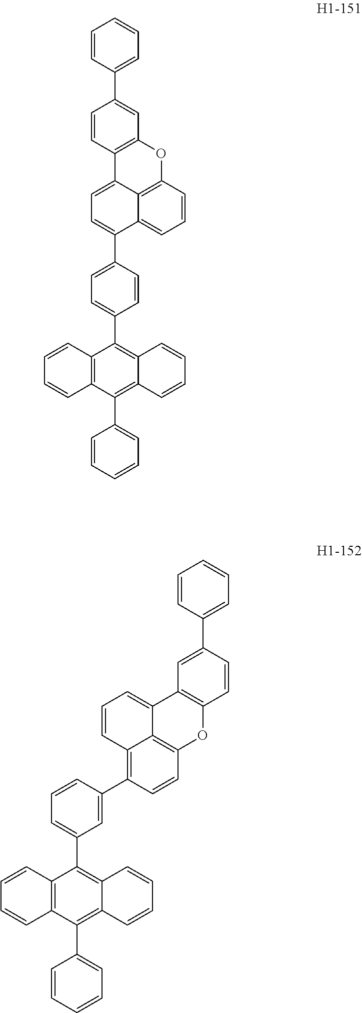 [SMILES: c1ccc(-c2ccc3c(c2)Oc2cccc4c(-c5ccc(-c6c7ccccc7c(-c7ccccc7)c7ccccc67)cc5)ccc-3c24)cc1 ; c1ccc(-c2ccc3c(c2)-c2cccc4c(-c5cccc(-c6c7ccccc7c(-c7ccccc7)c7ccccc67)c5)ccc(c24)O3)cc1]

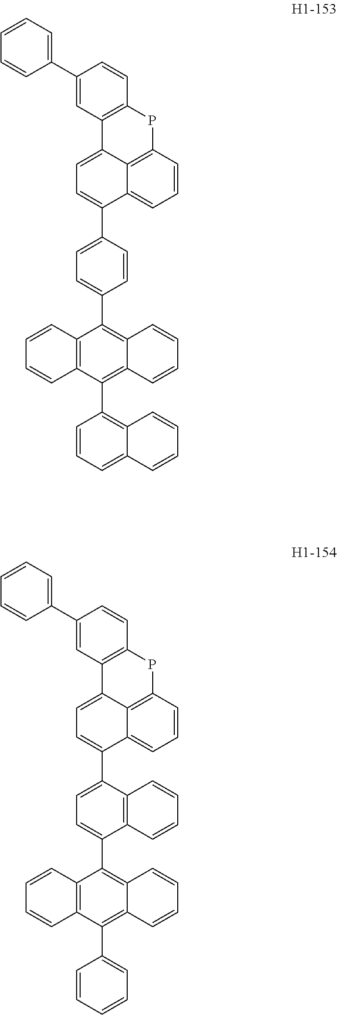

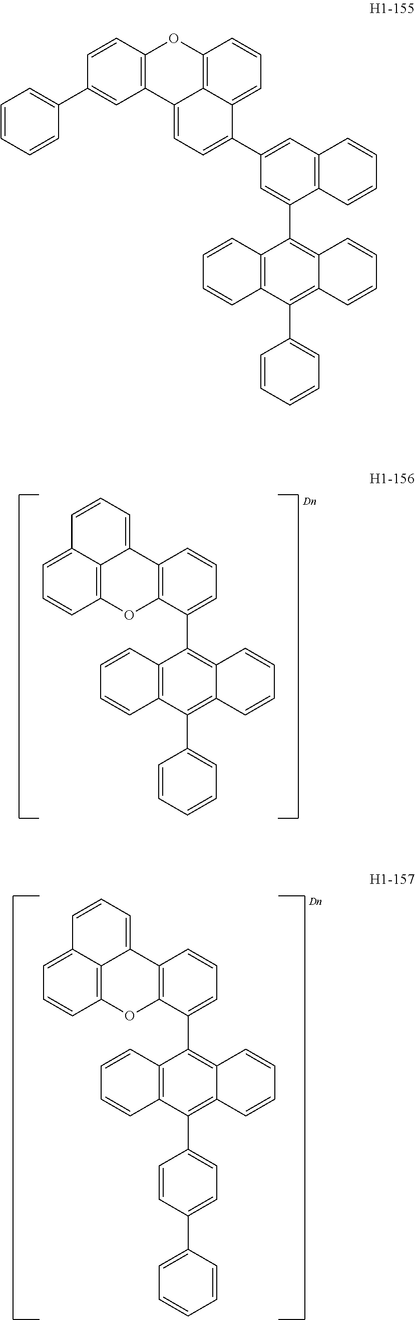

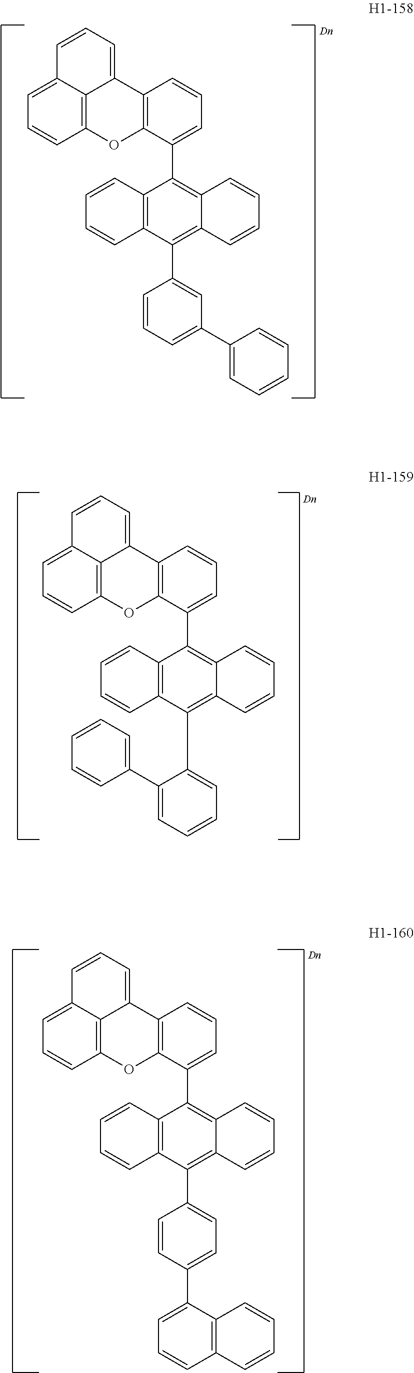

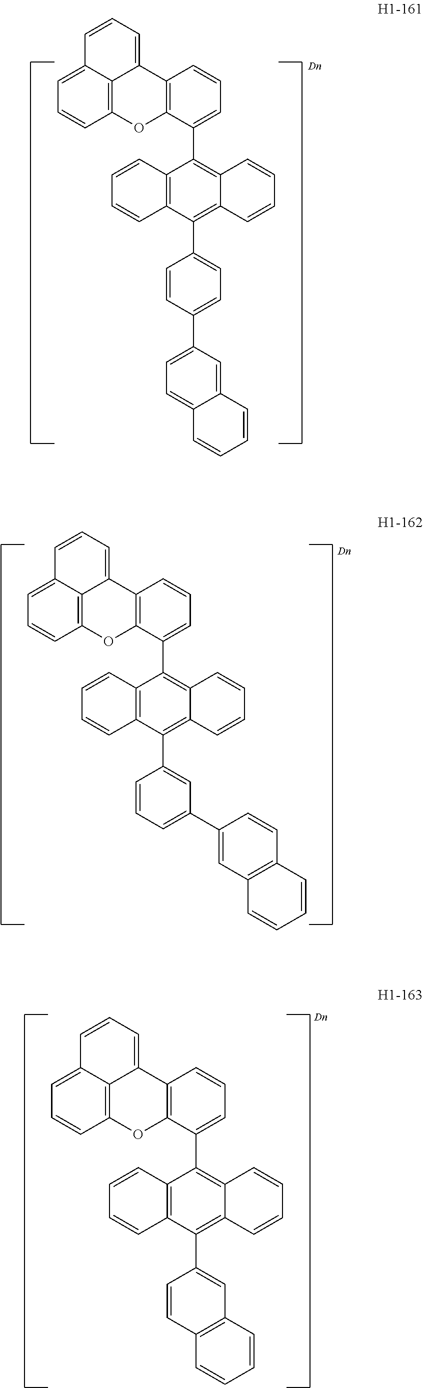

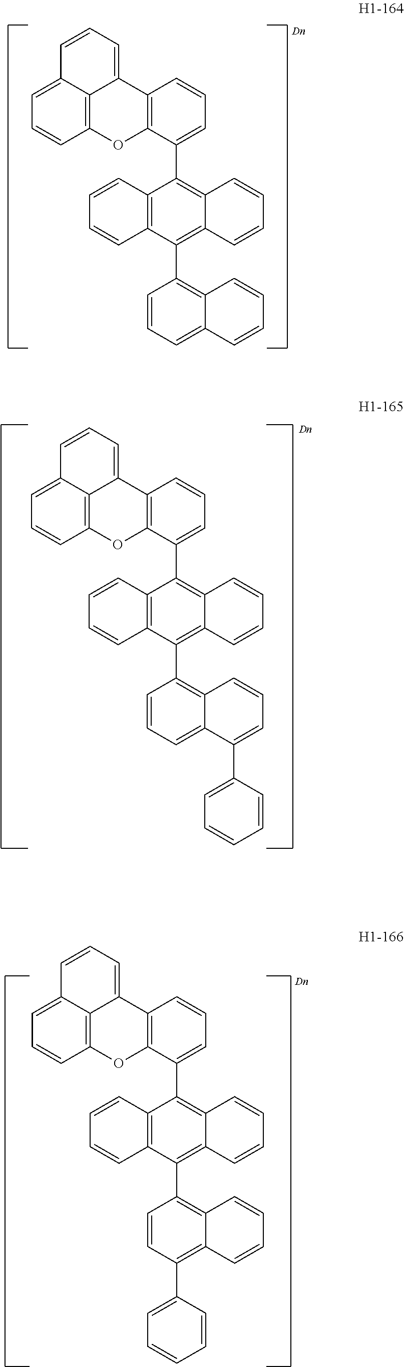

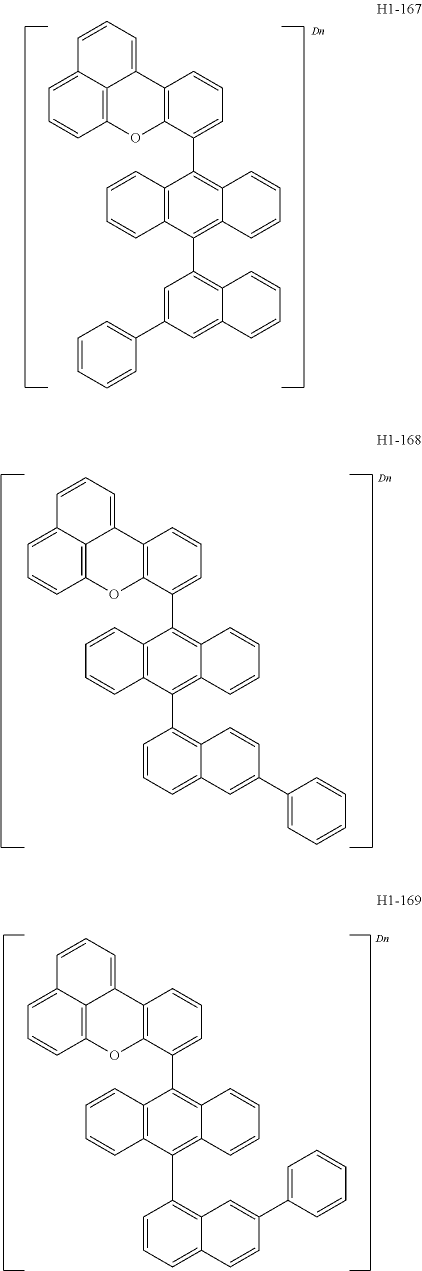

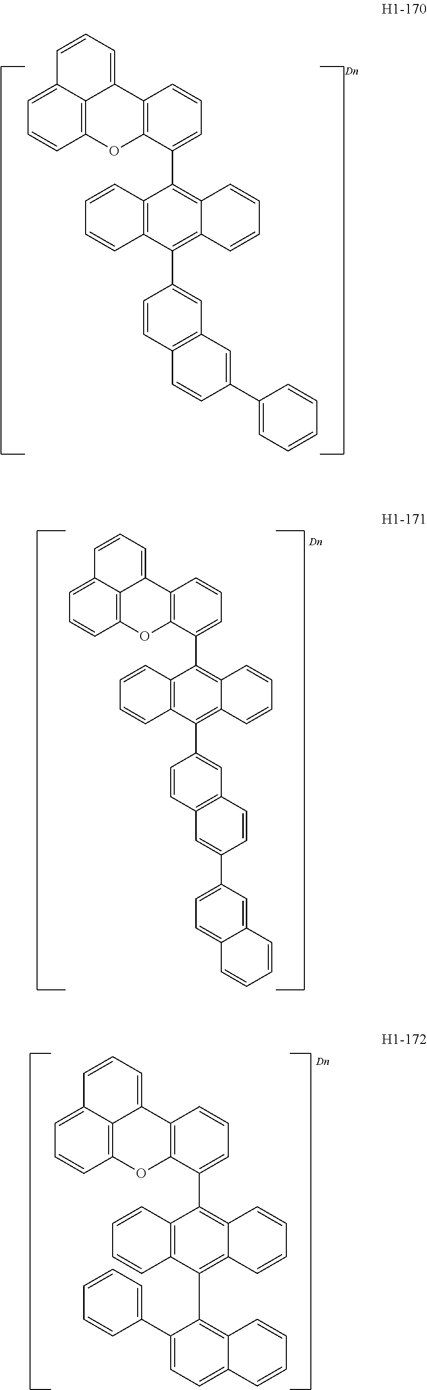

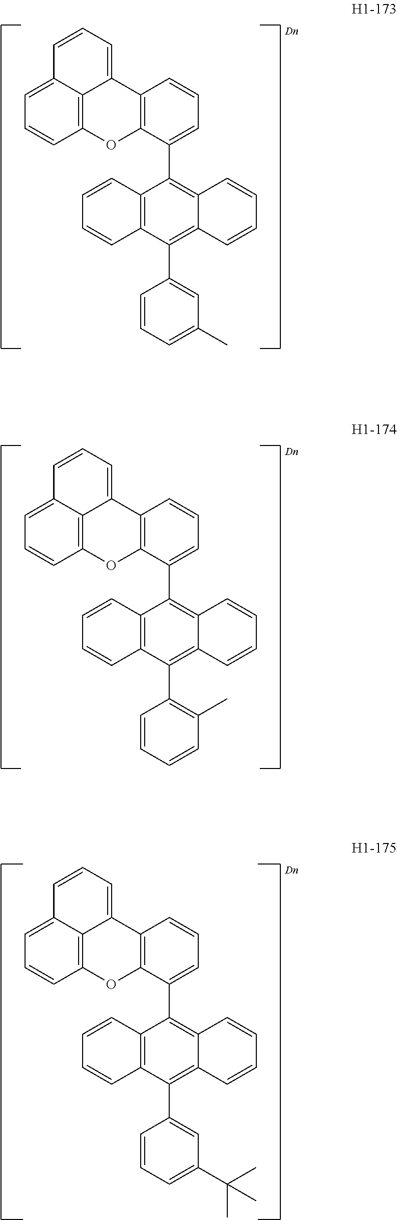

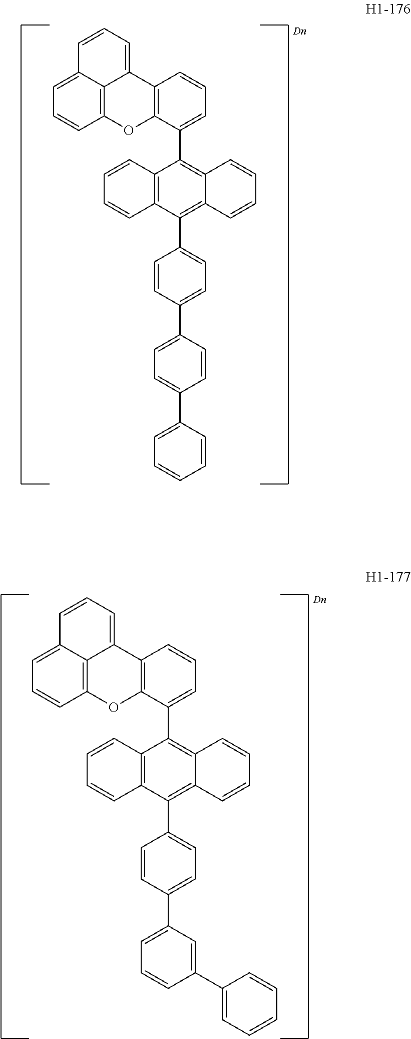

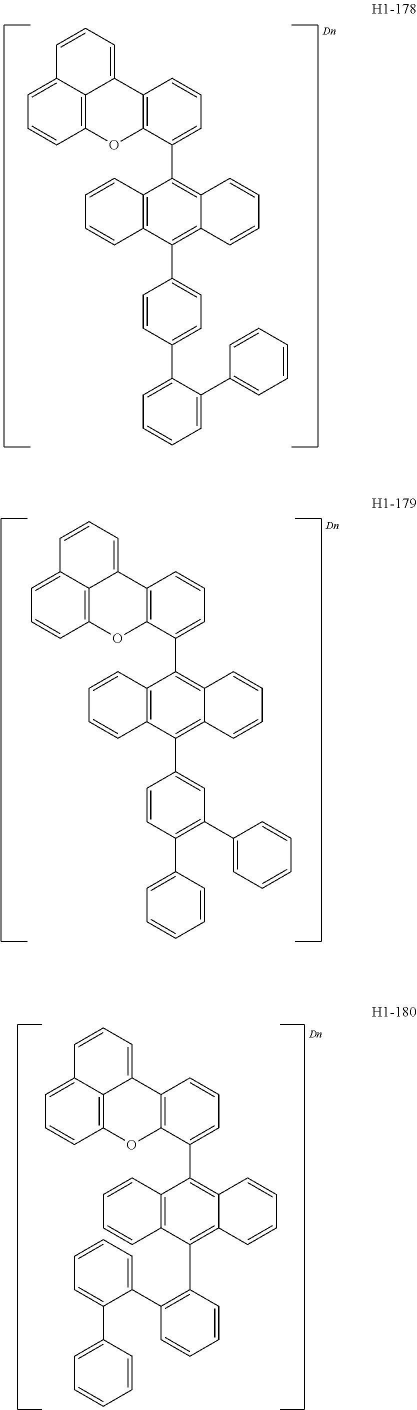

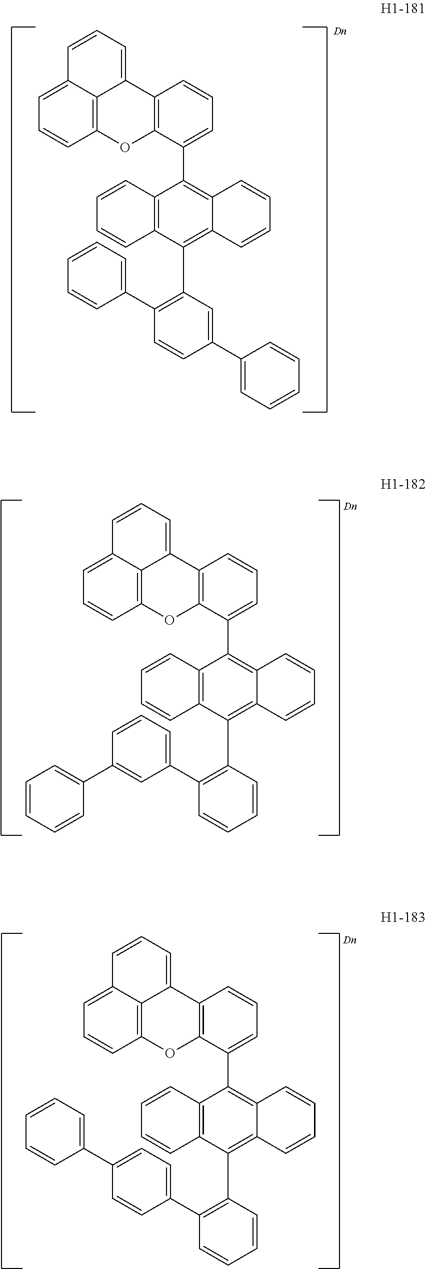

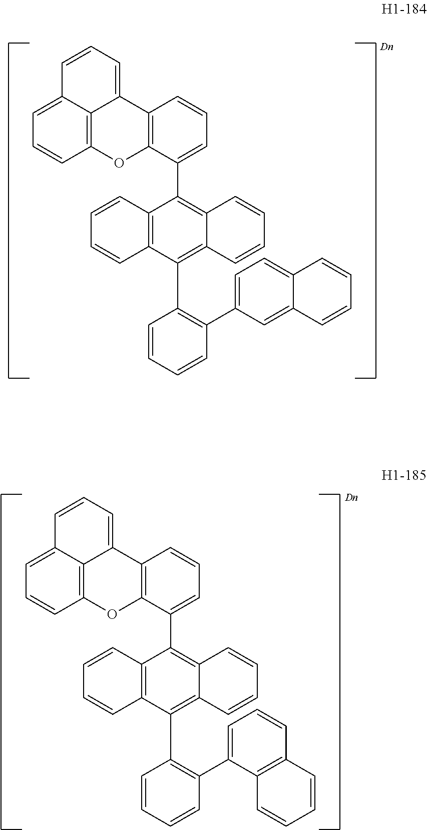

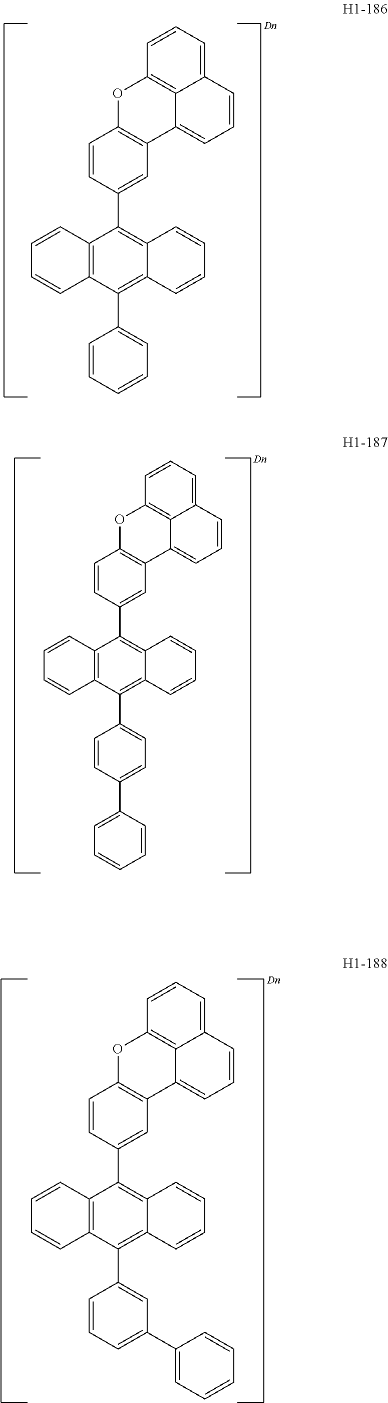

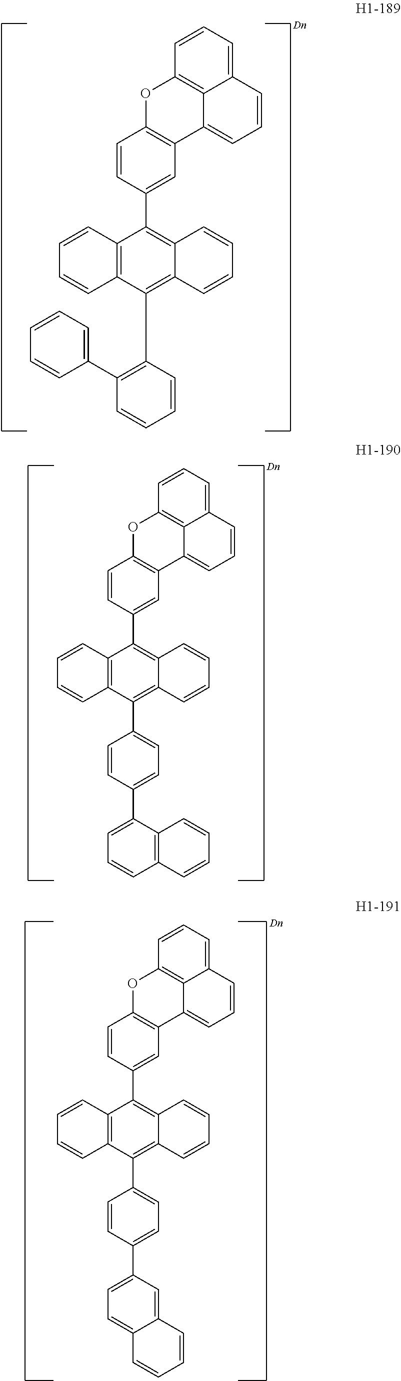

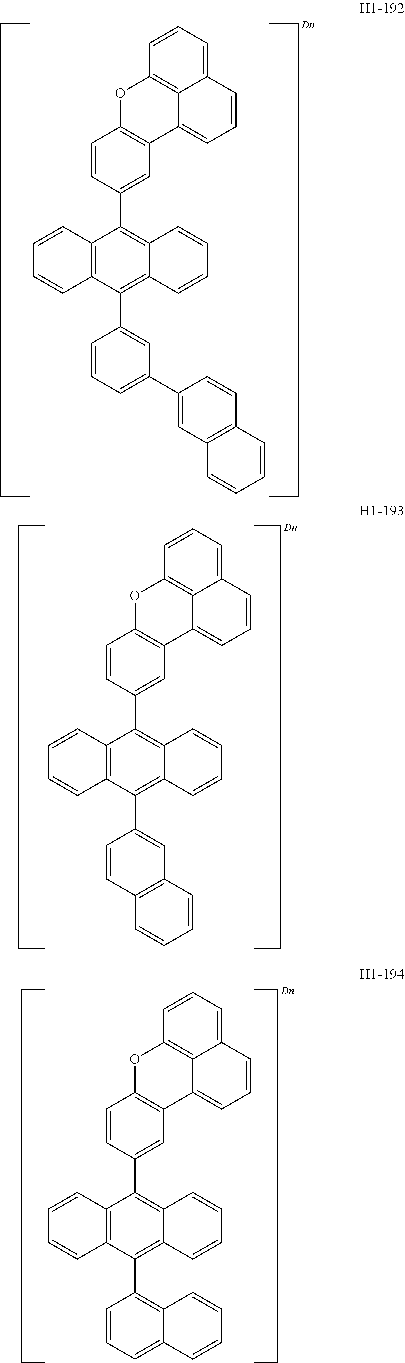

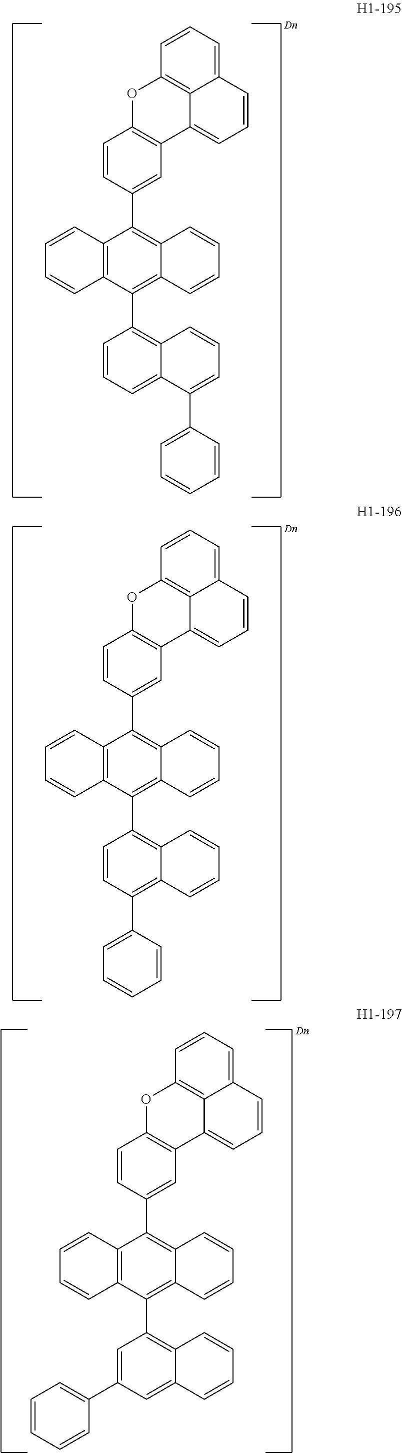

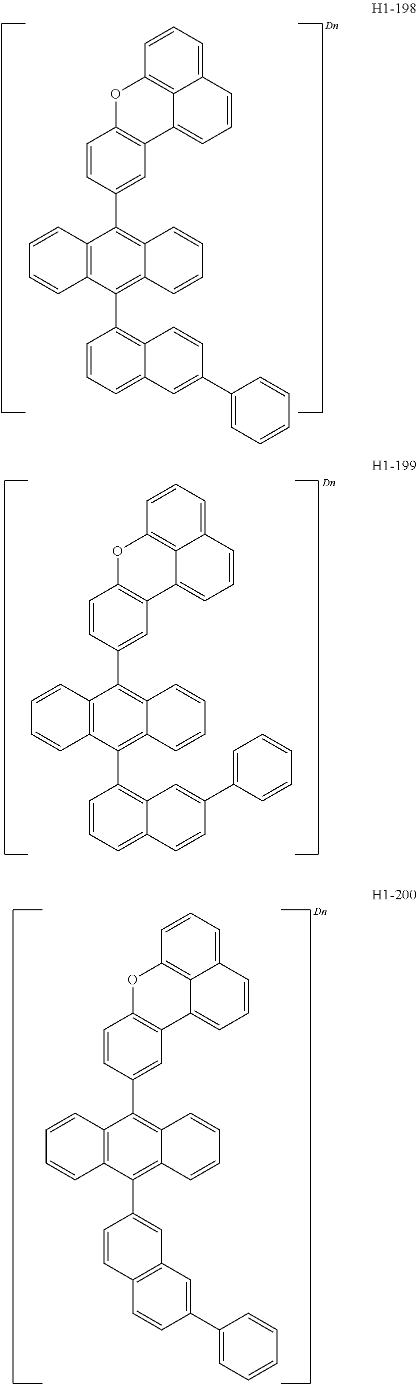

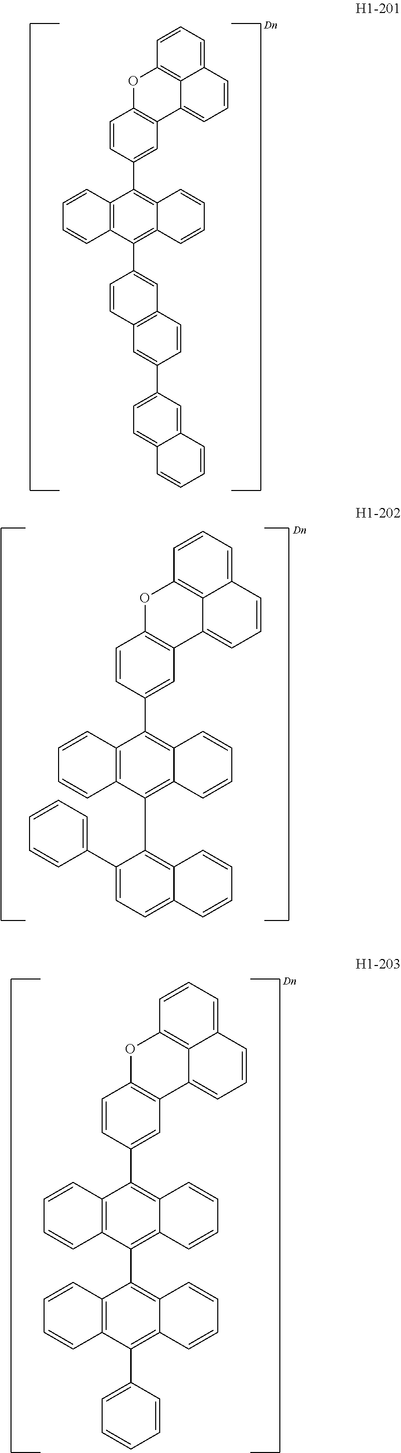

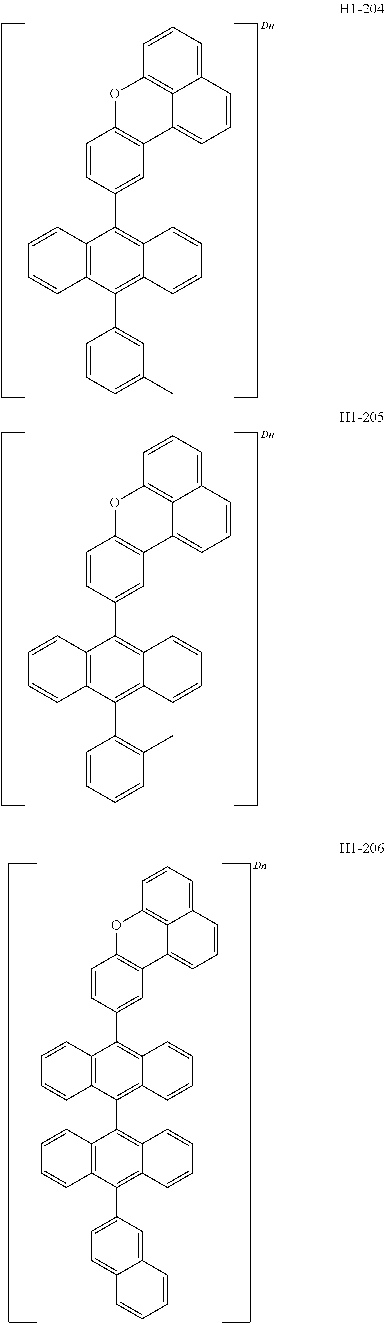

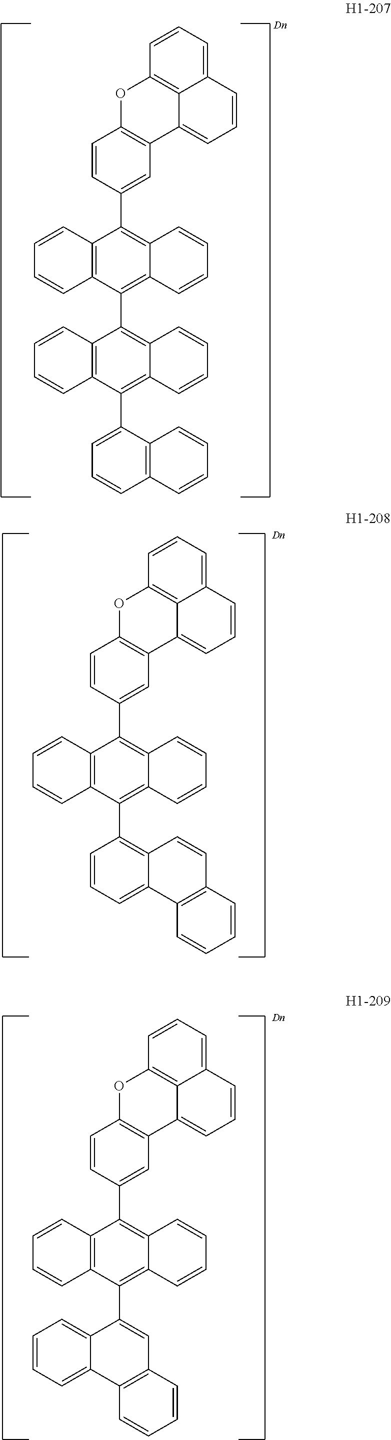

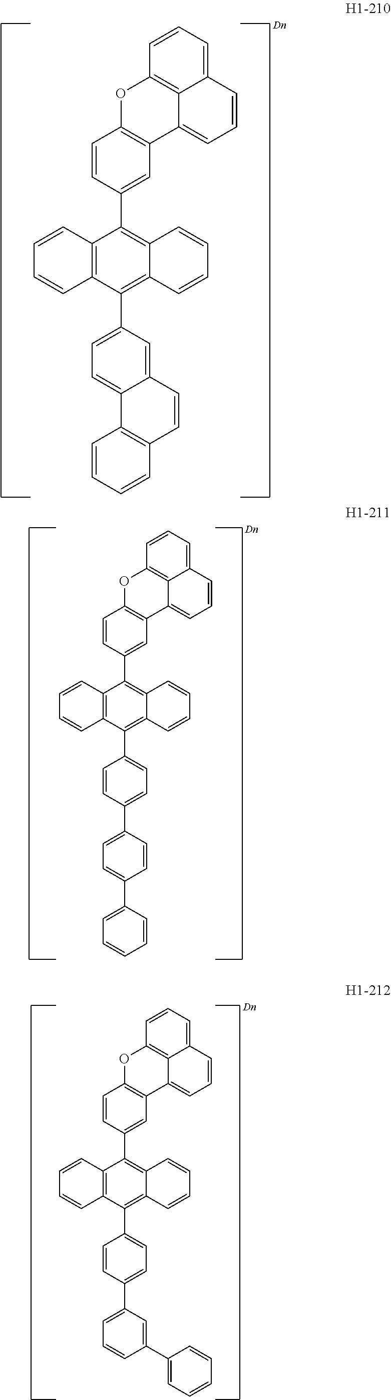

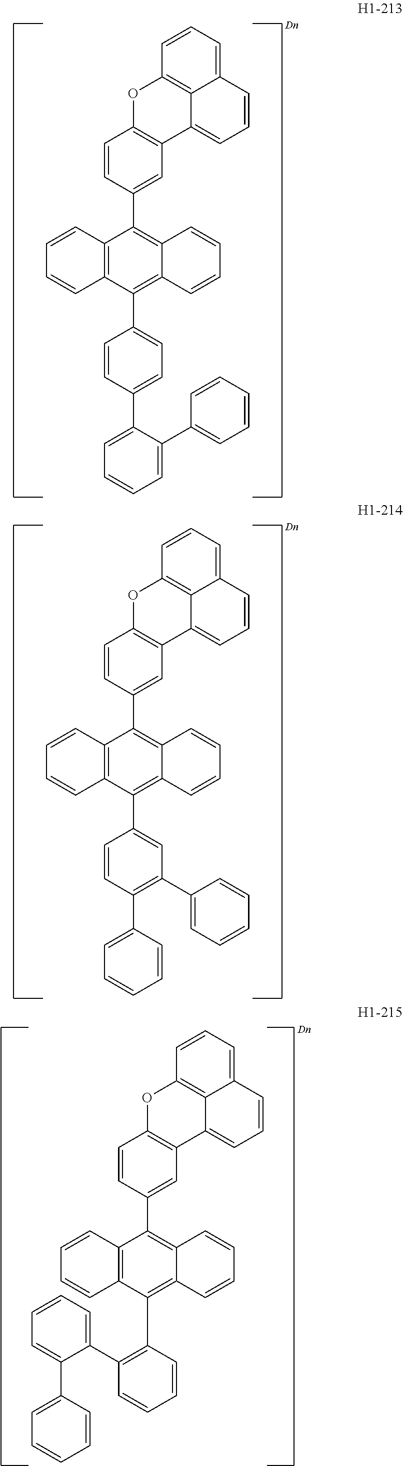

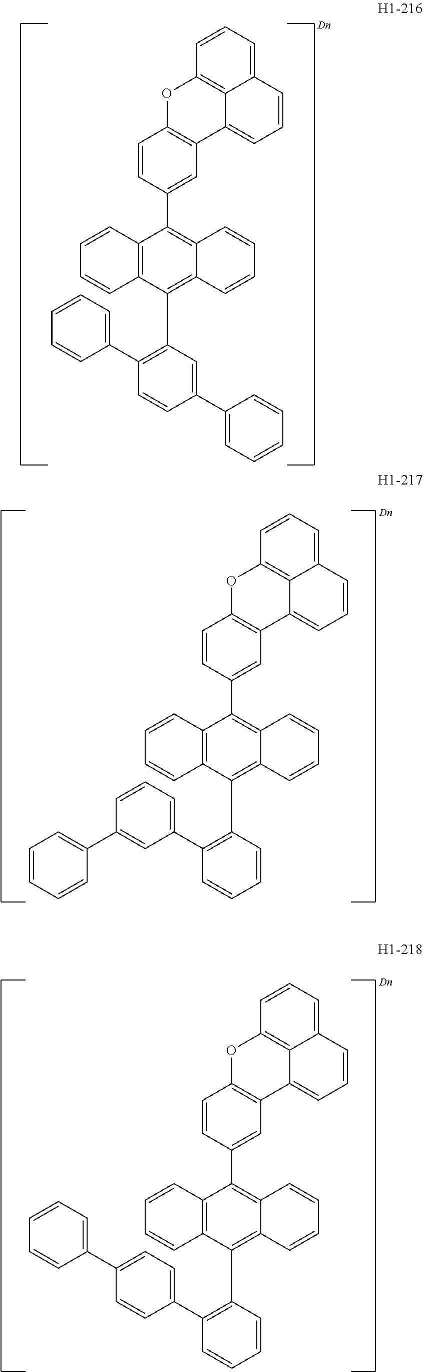

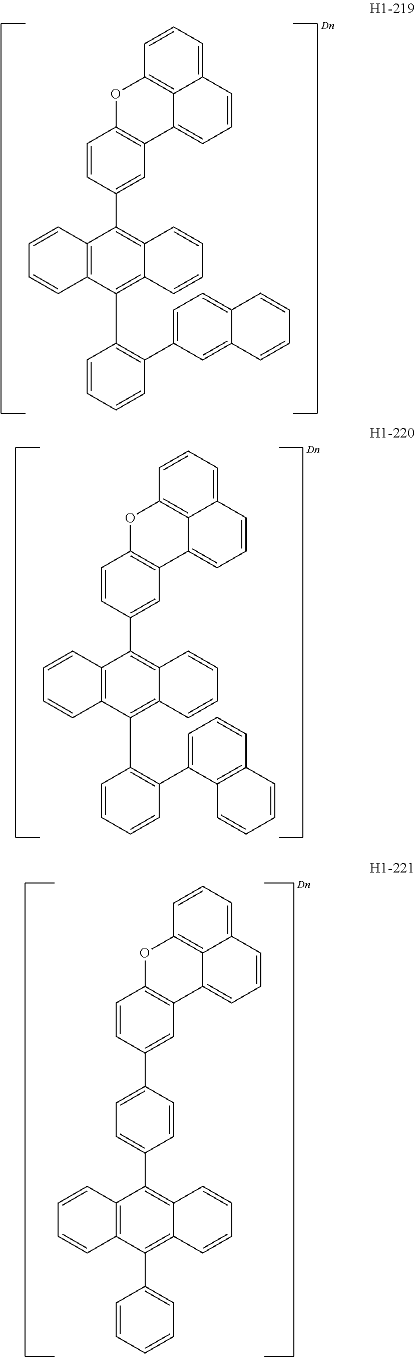

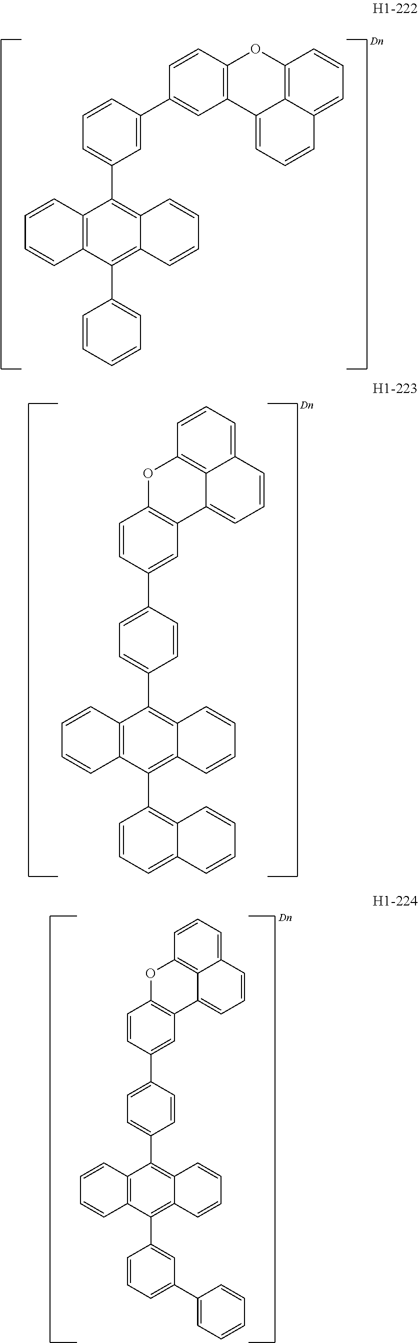

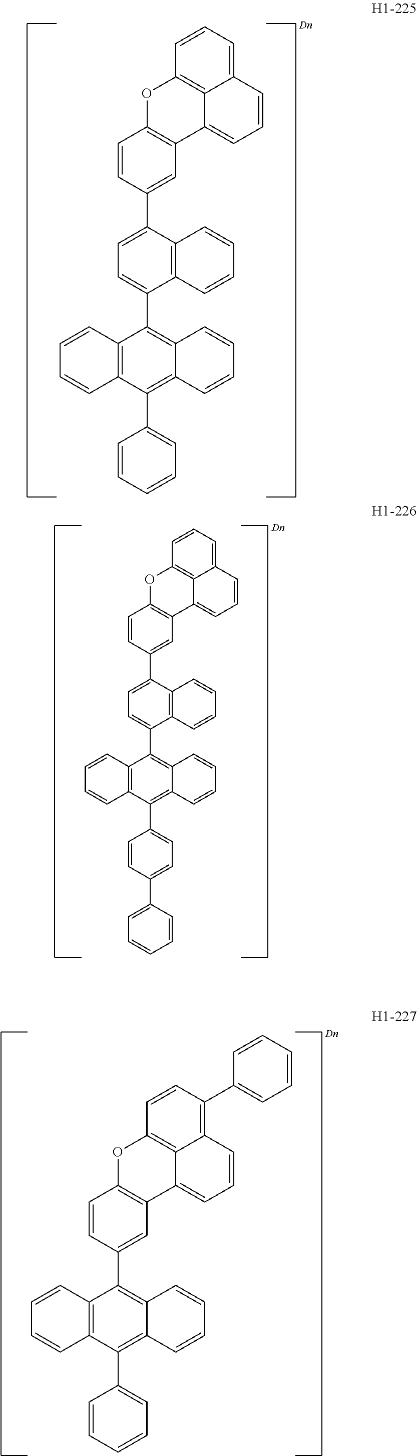

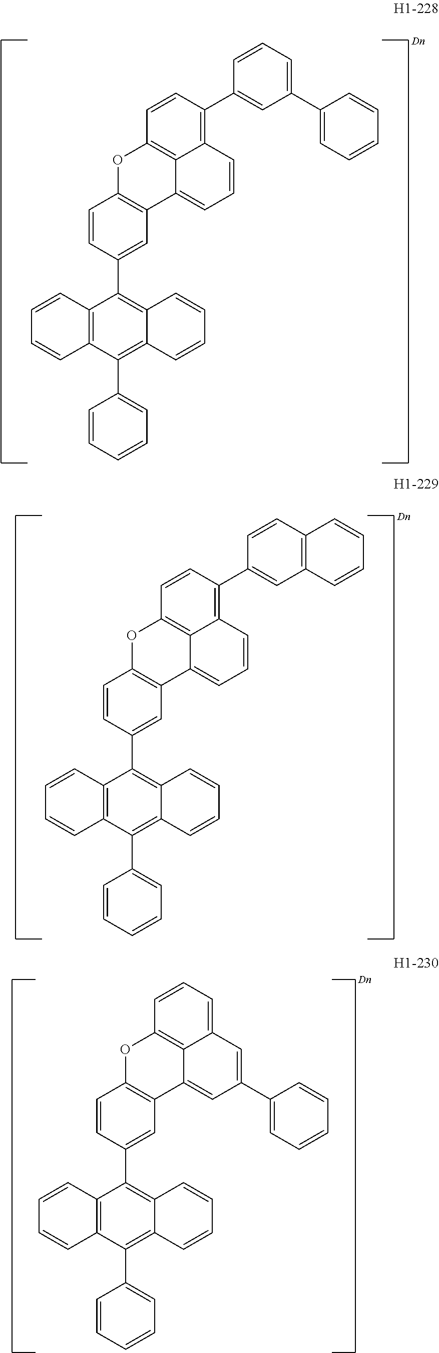

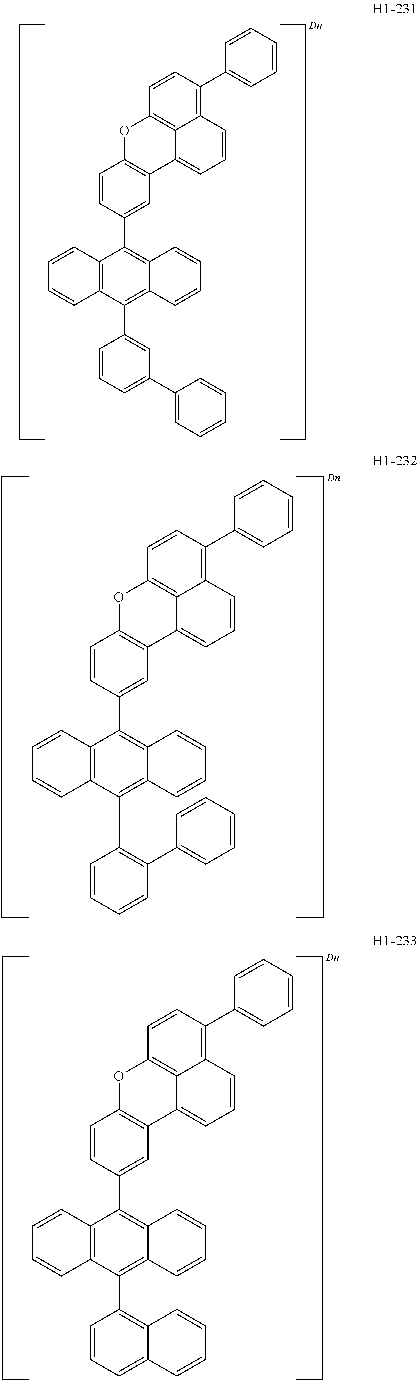

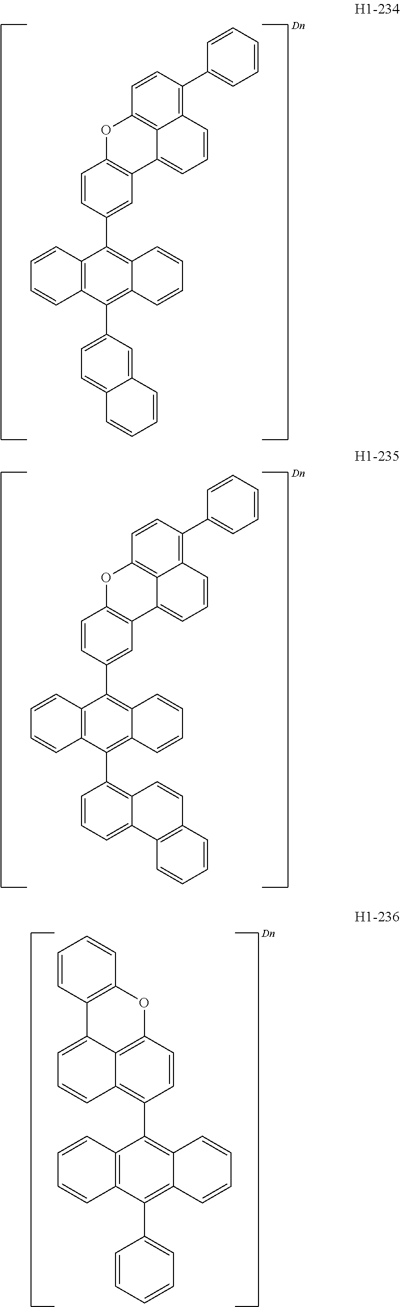

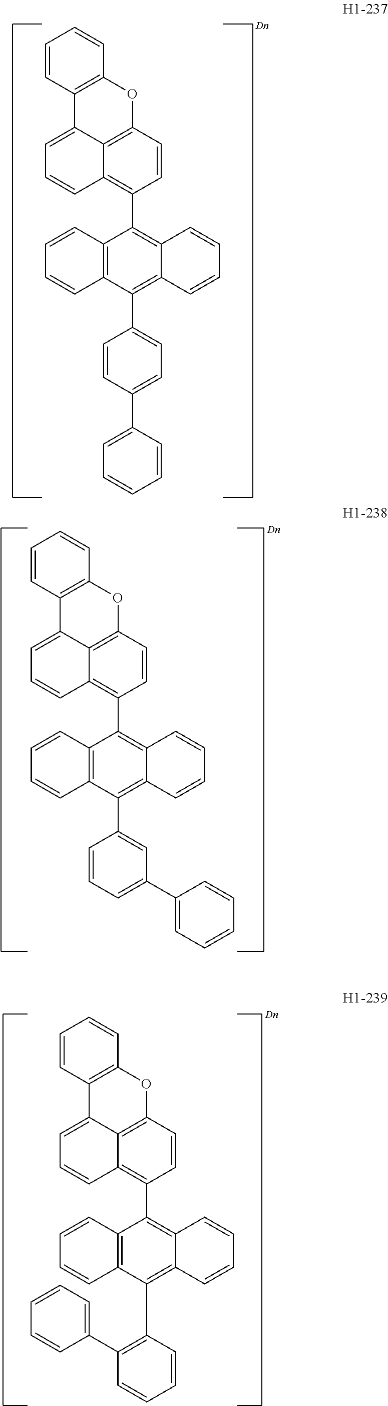

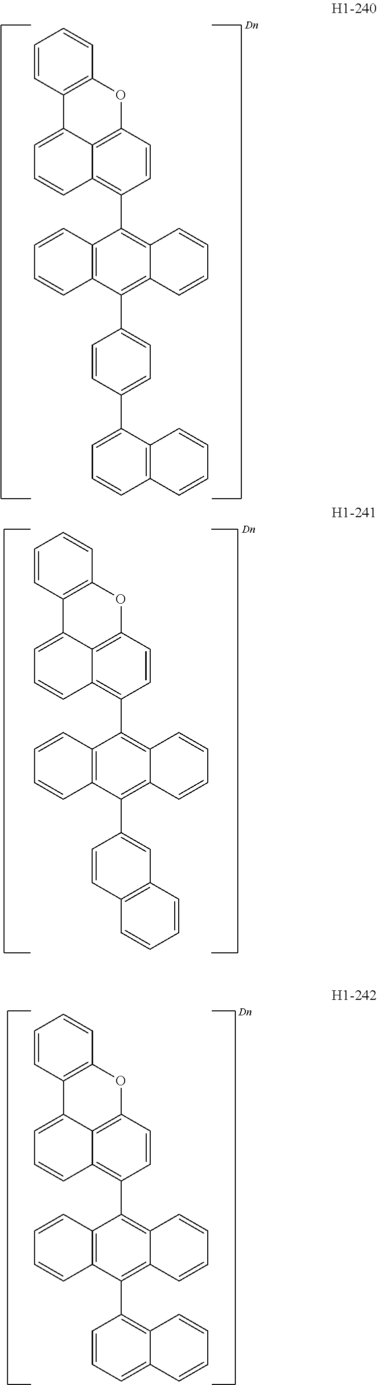

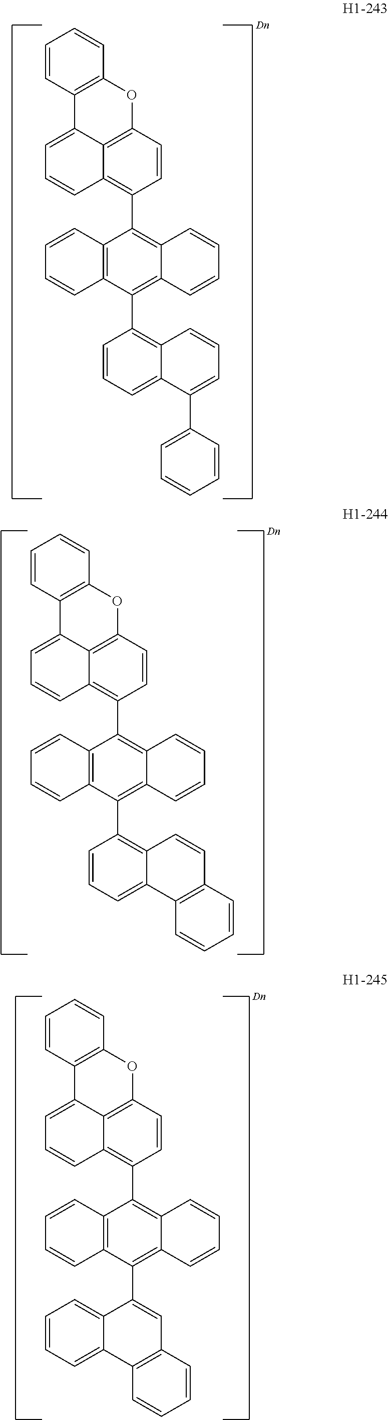

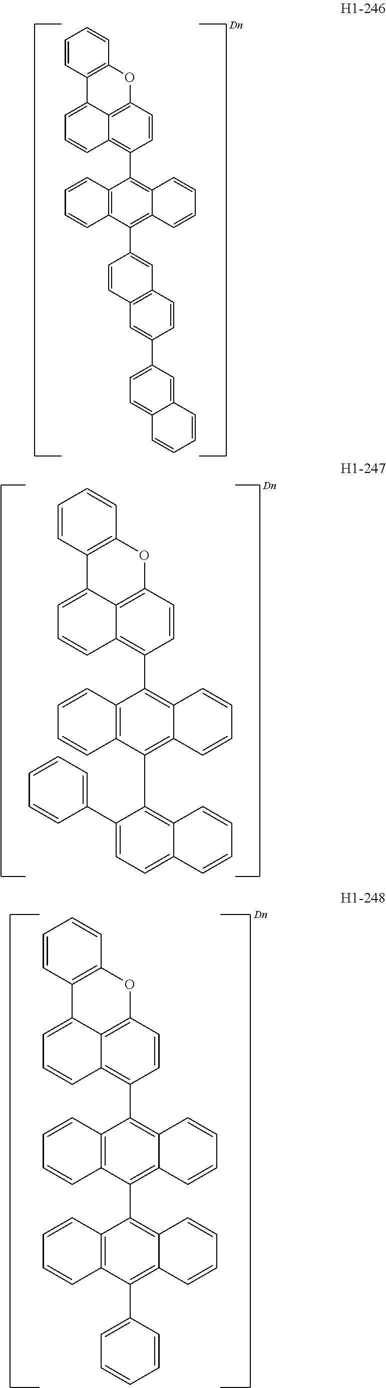

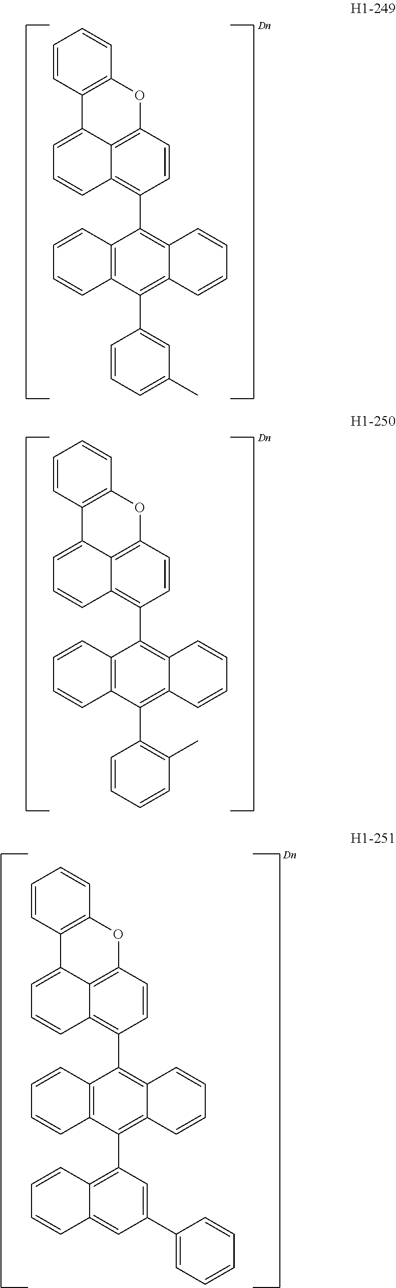

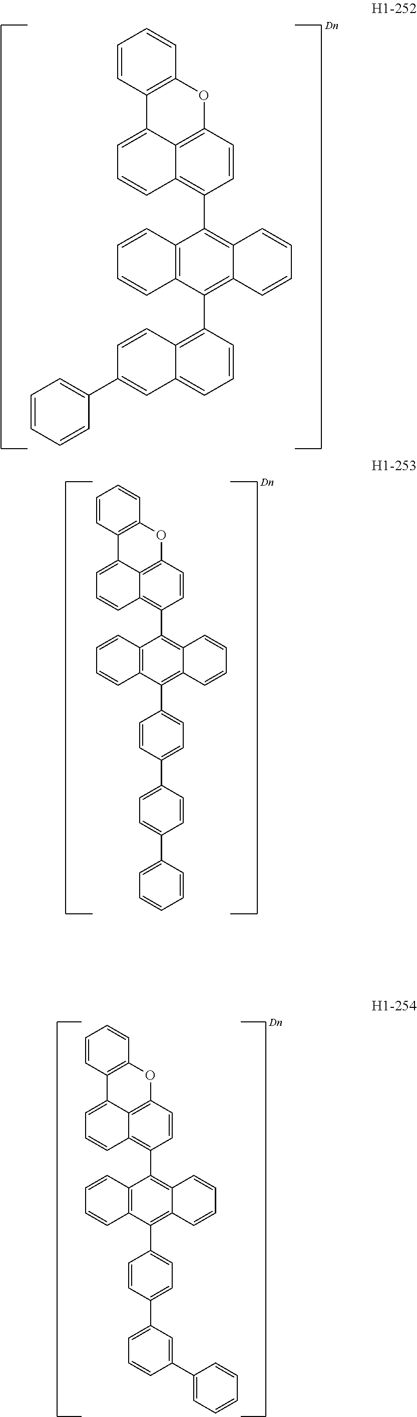

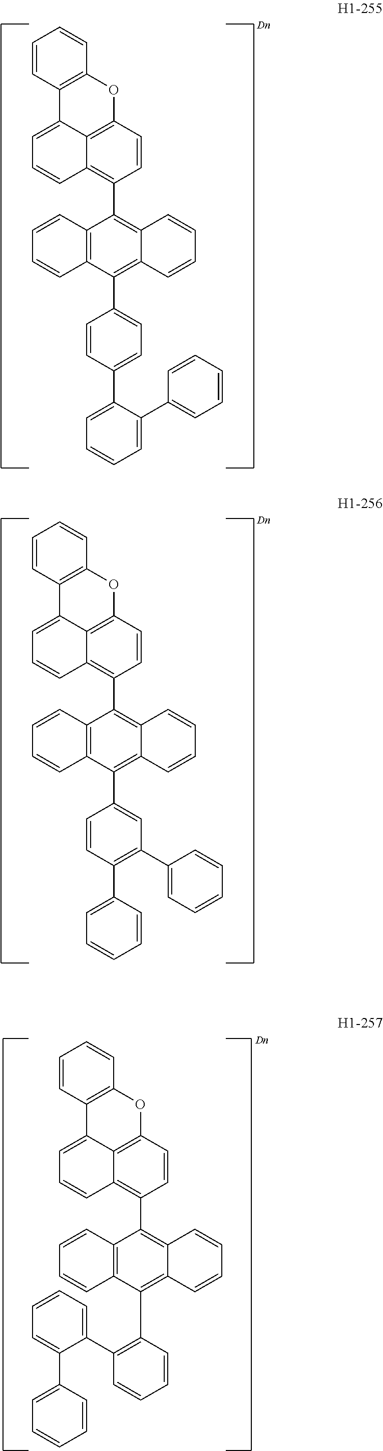

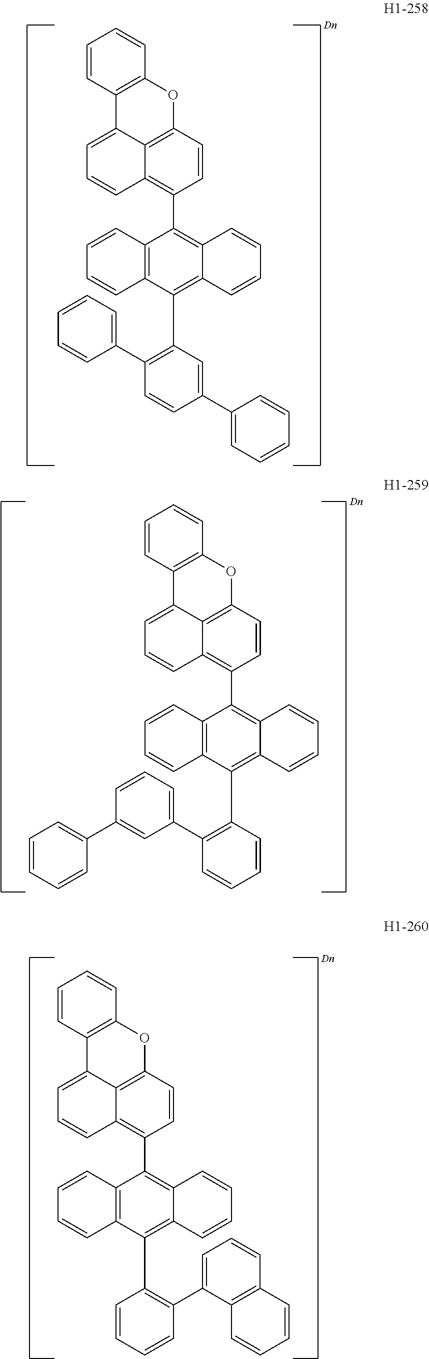

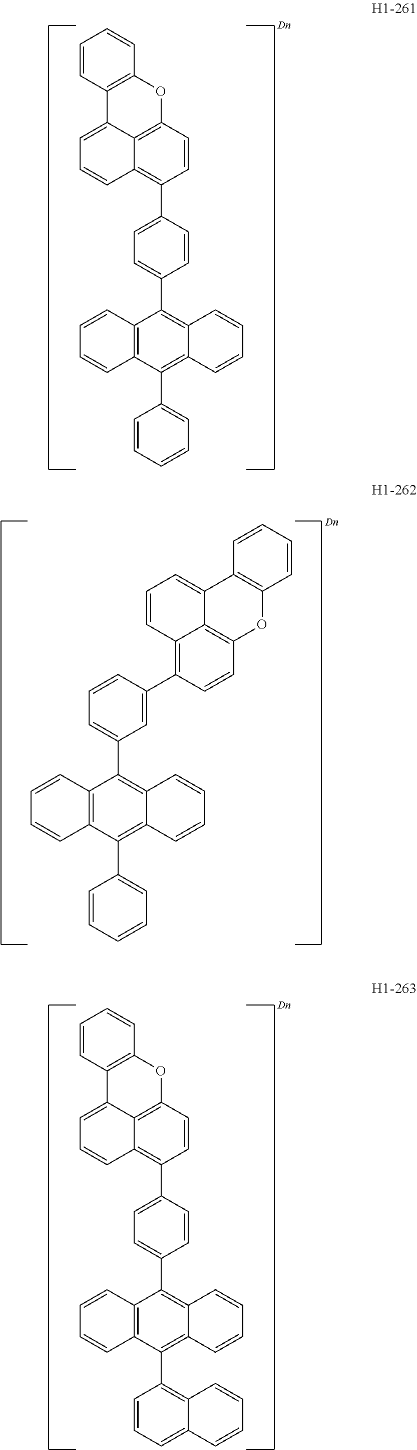

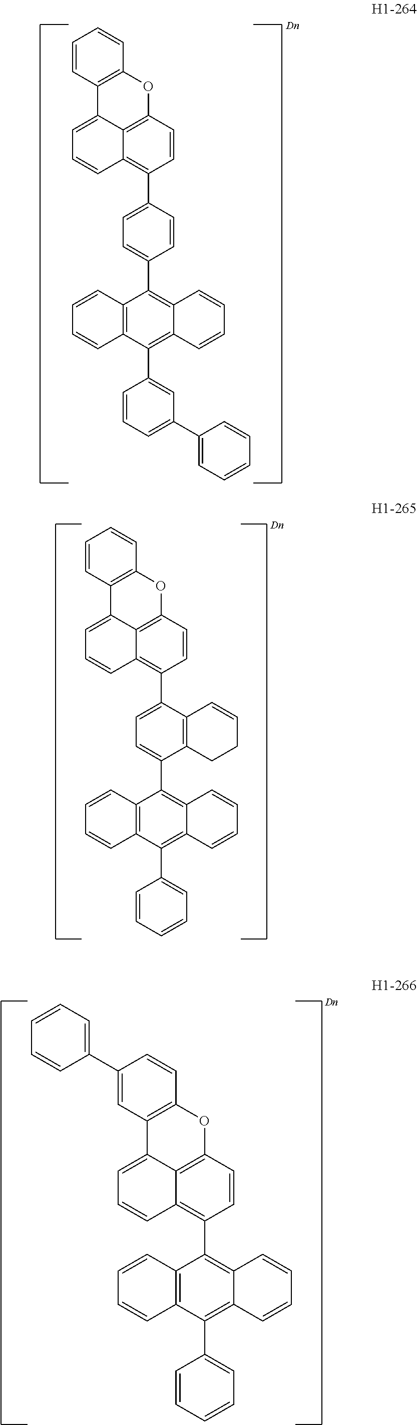

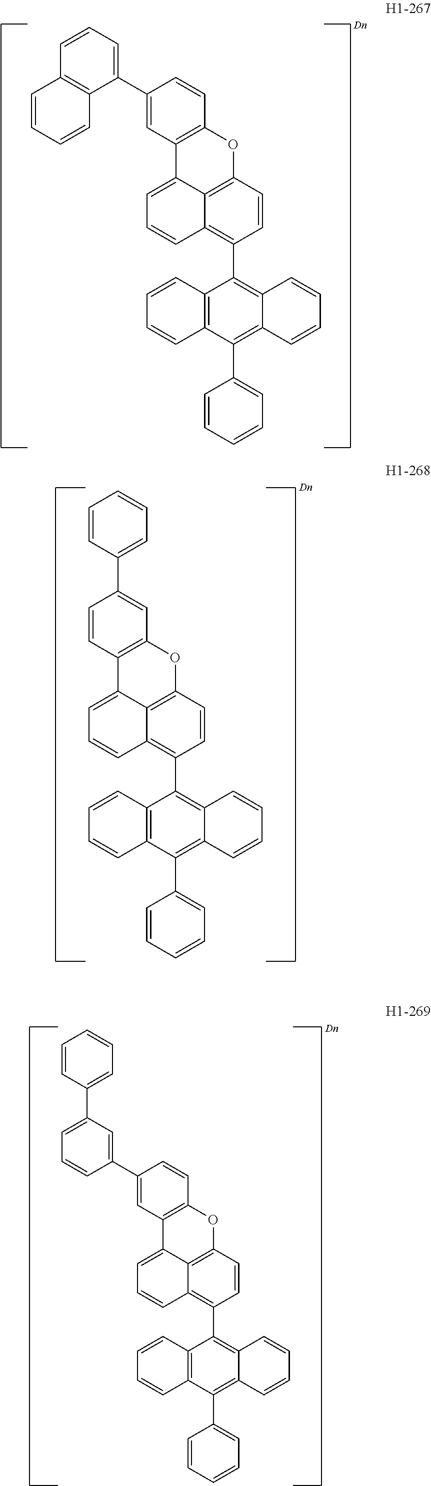

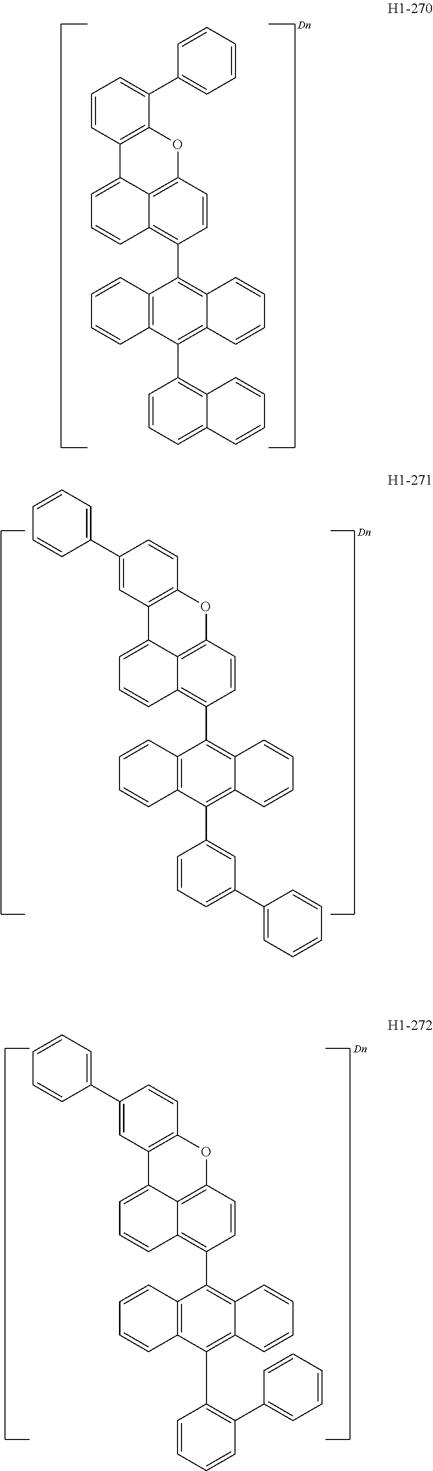

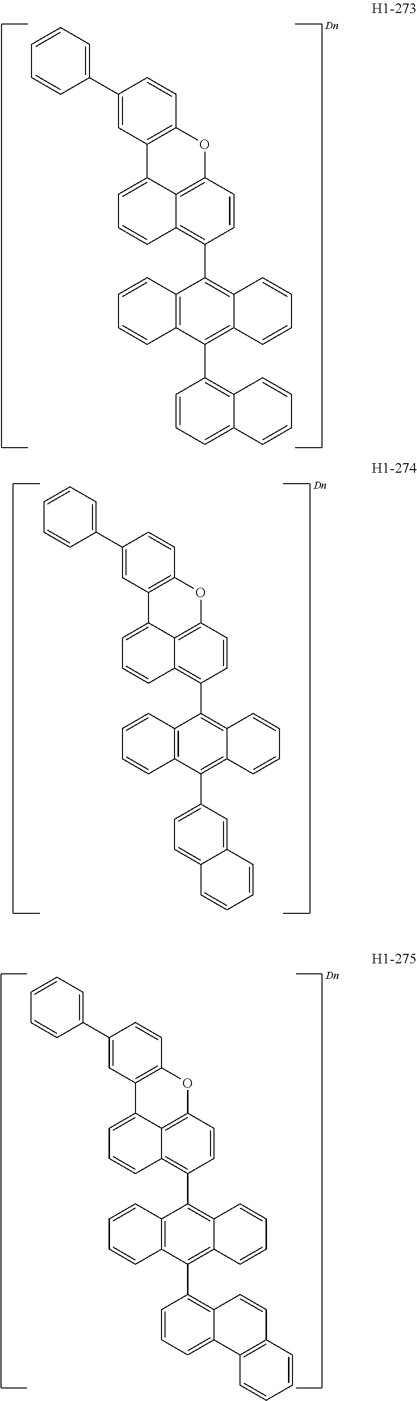

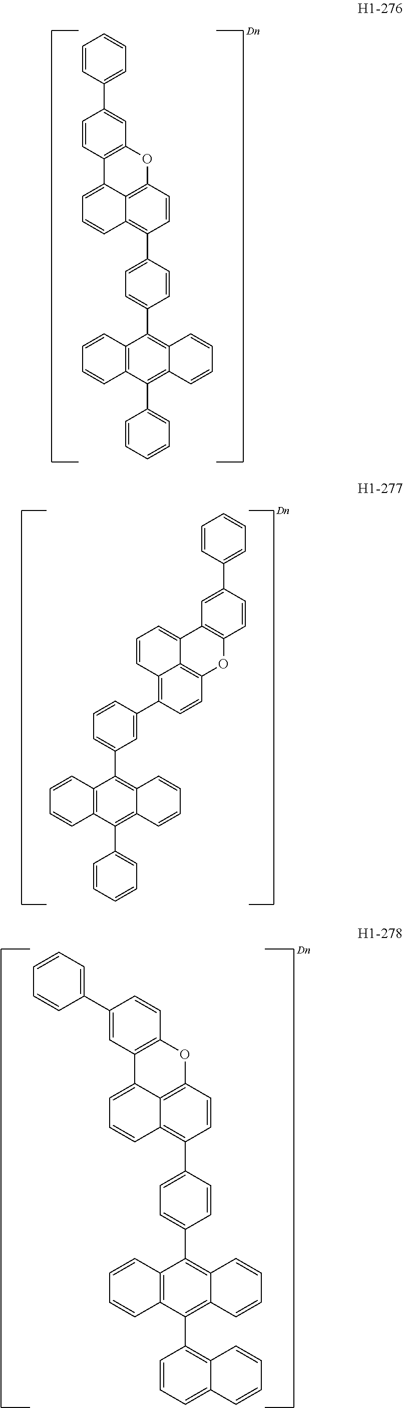

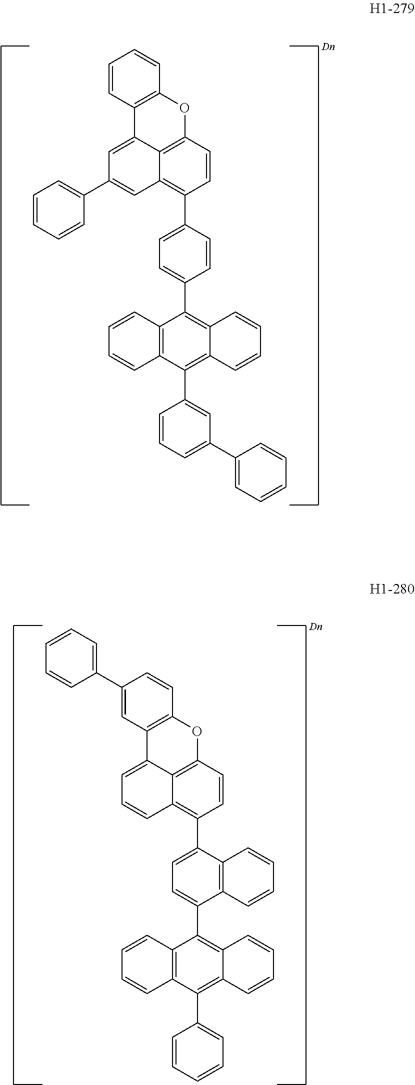

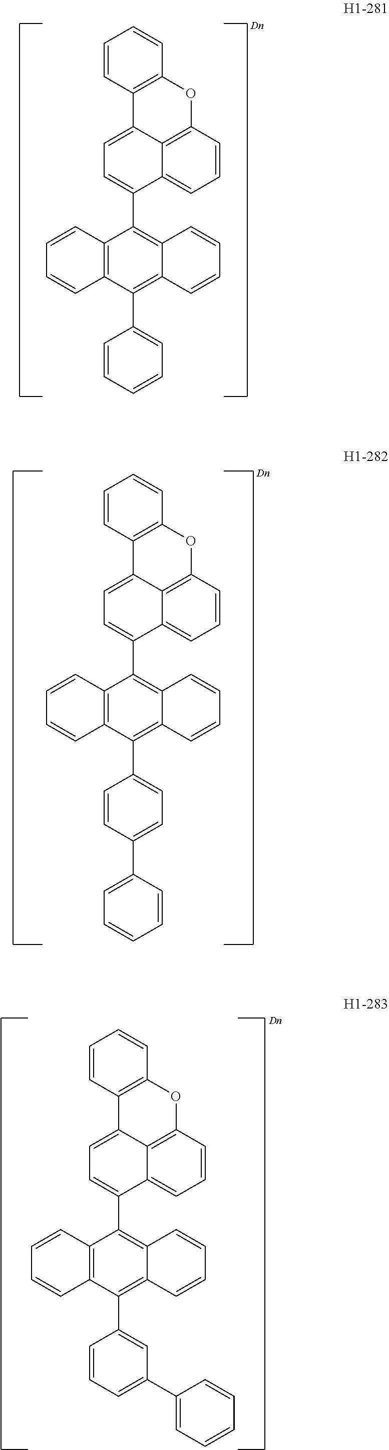

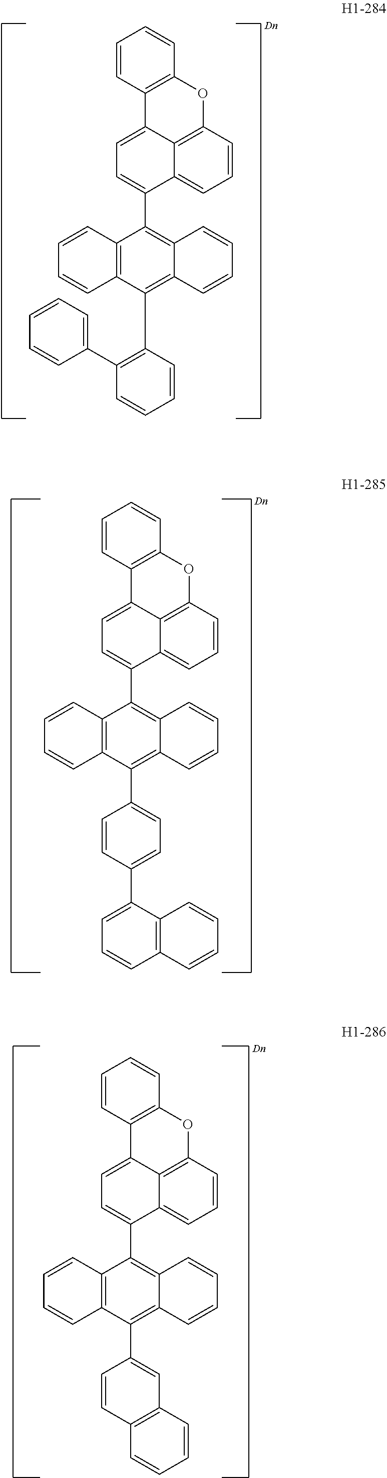

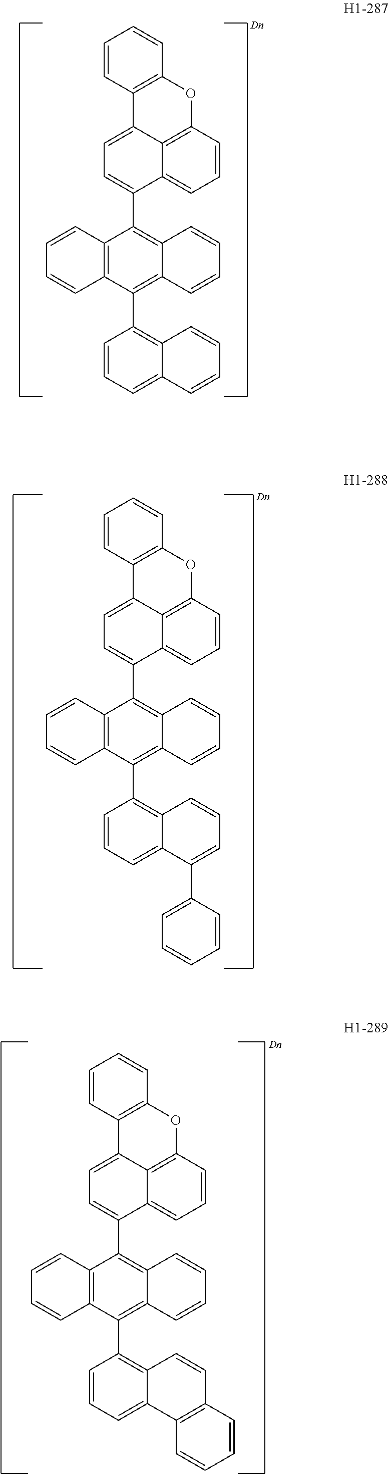

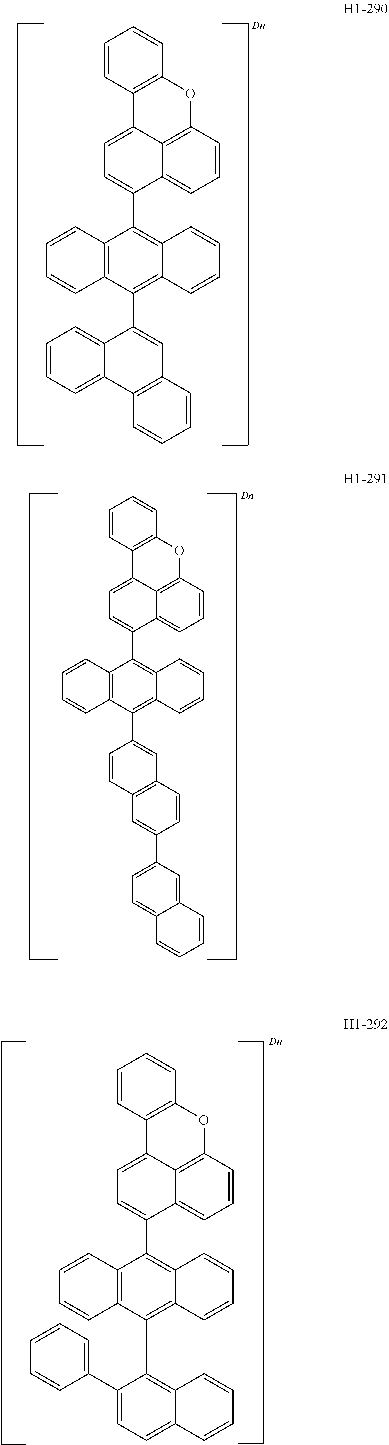

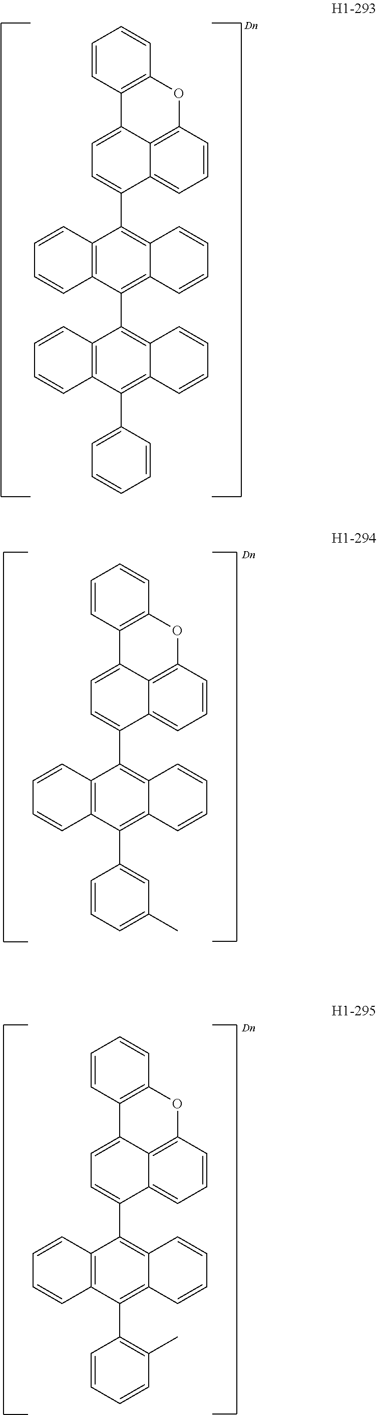

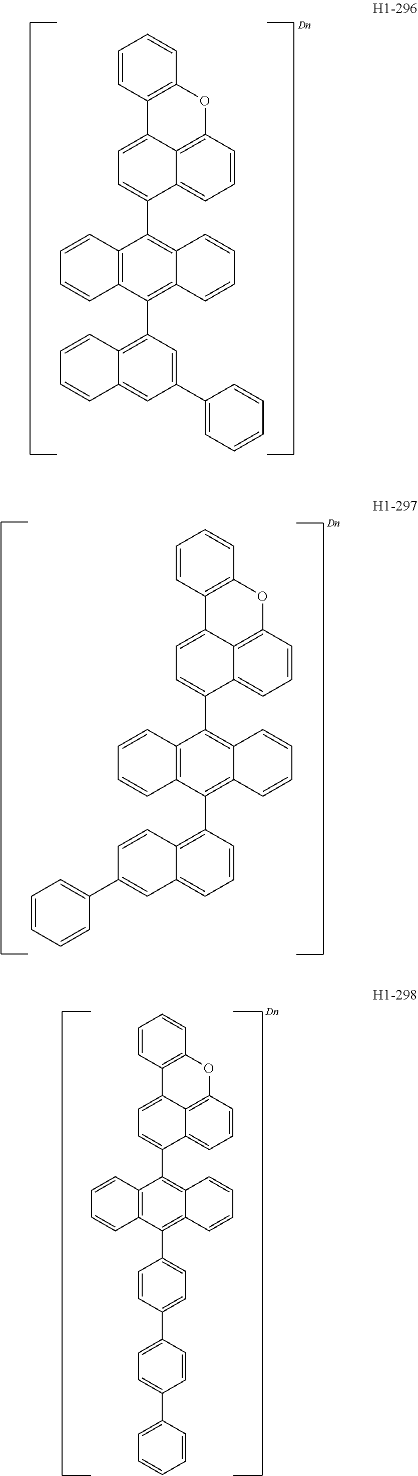

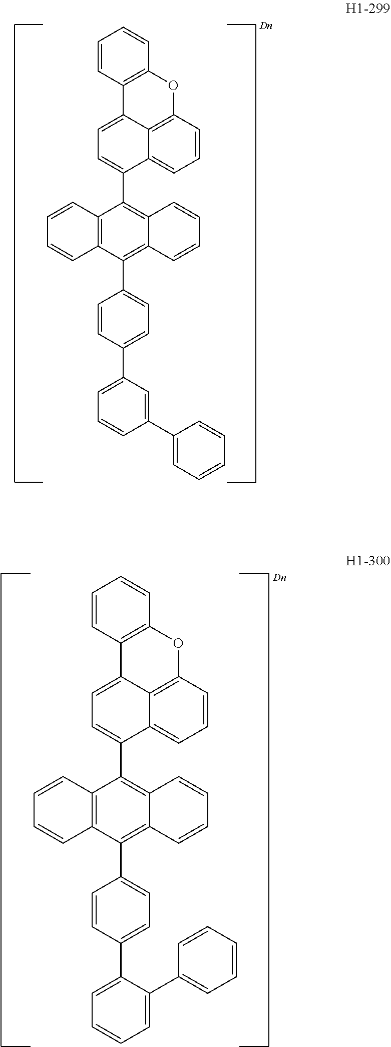

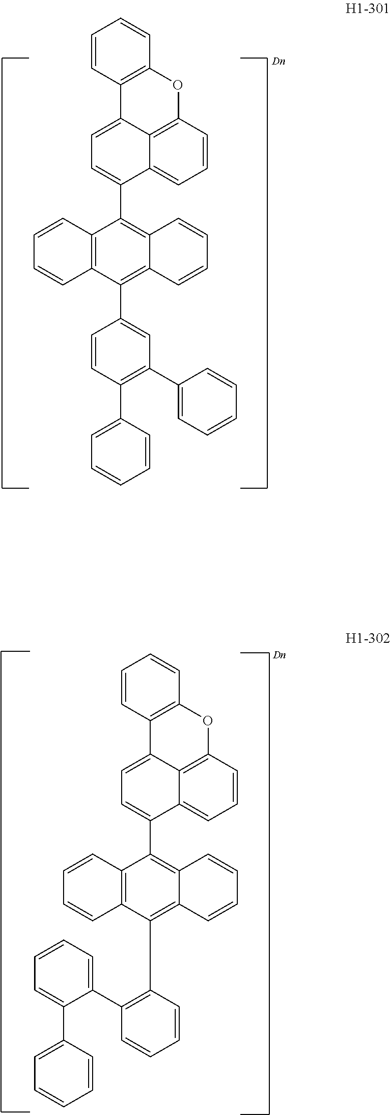

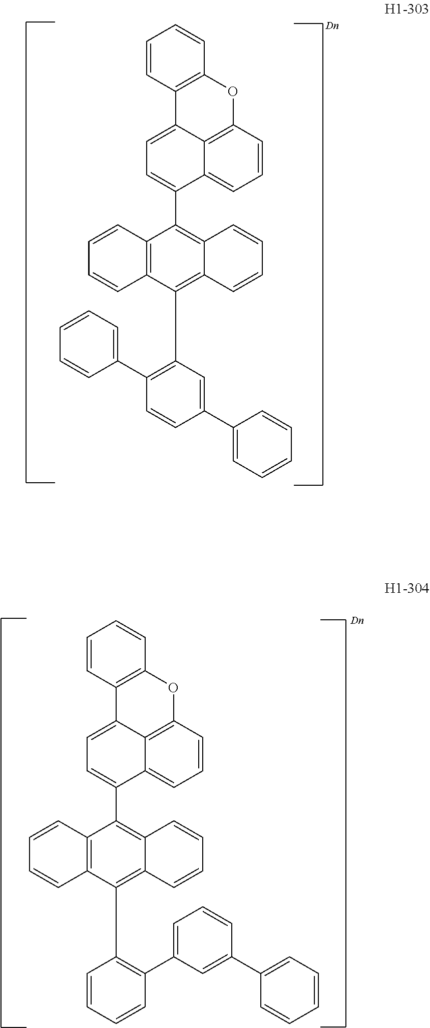

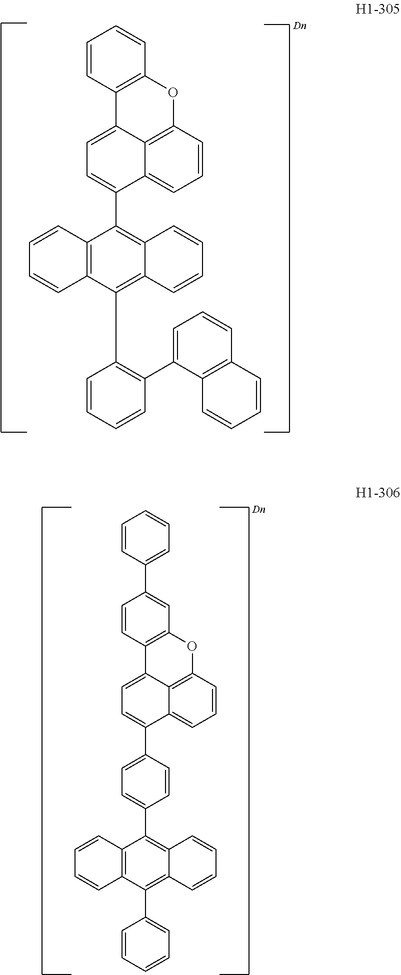

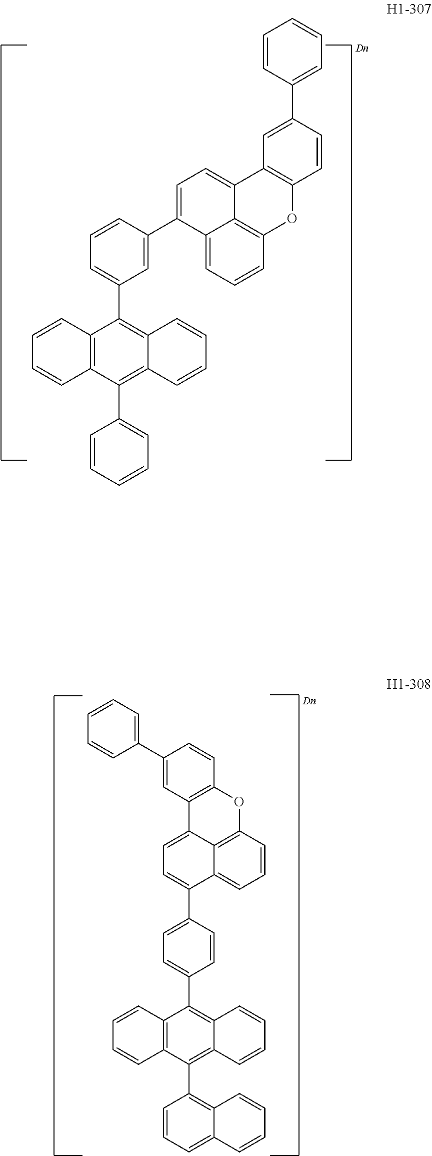

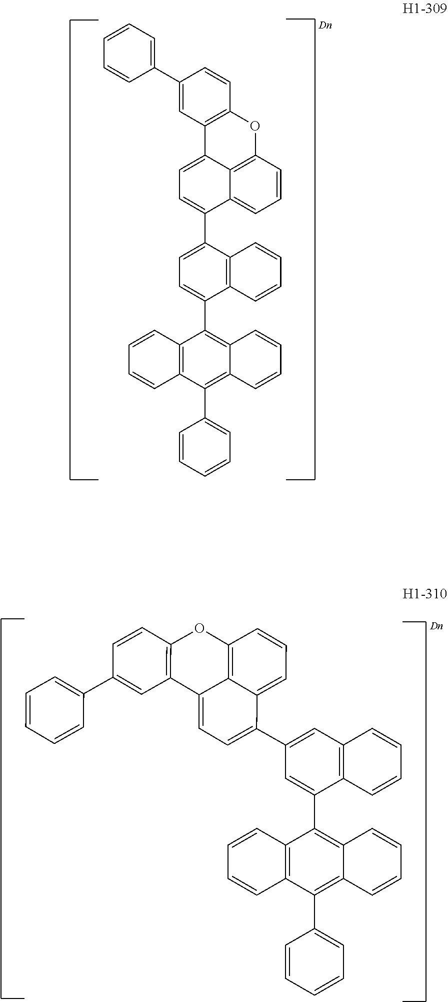

wherein “Dₙ” in the above compounds means that n number of hydrogens is replaced by deuterium, wherein n represents an integer of 1 or more and the upper limit of n is determined by the number of hydrogens that can be substituted in each compound.

The second host compound as another host material according to one embodiment may be represented by the following Formula 2.

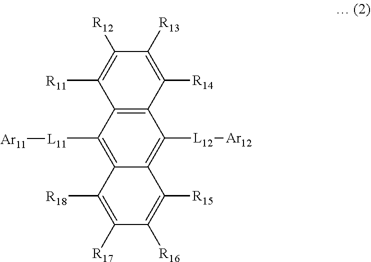

In Formula 2,
L₁₁ and L₁₂ each independently represent a single bond, a substituted or unsubstituted (C₆-C₃₀)arylene, or a substituted or unsubstituted (3- to 30-membered)heteroarylene;
Ar₁₁ and Ar₁₂ each independently represent a substituted or unsubstituted (C₆-C₃₀)aryl or a substituted or unsubstituted (3- to 30-membered)heteroaryl; and
R₁₁ to R₁₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of (C₃-C₃₀) aliphatic ring and (C₆-C₃₀) aromatic ring.

In one embodiment, R₁₁ to R₁₈ each independently may be hydrogen or deuterium.

In one embodiment, L₁₁ and L₁₂ each independently may be a single bond, or a substituted or unsubstituted (C₆-C₃₀)arylene, preferably a single bond, or a substituted or unsubstituted (C₆-C₂₅)arylene, more preferably (C₆-C₁₃)arylene unsubstituted or substituted with deuterium or (C₆-C₃₀)aryl. For example, L₁₁ and L₁₂ each independently may be a single bond, a substituted or unsubstituted phenylene, a substituted or unsubstituted biphenylene, a substituted or unsubstituted naphthylene, or a substituted or unsubstituted phenanthrenylene, wherein the substituents may be replaced with one or more deuterium atoms.

In one embodiment, Ar₁₁ and Ar₁₂ each independently may be a substituted or unsubstituted (C₆-C₃₀)aryl, or a substituted or unsubstituted (5- to 30-membered)heteroaryl, preferably a substituted or unsubstituted (C₆-C₂₅)aryl, or a substituted or unsubstituted (5- to 25-membered)heteroaryl, more preferably a substituted or unsubstituted (C₆-C₁₃)aryl, or a substituted or unsubstituted (5- to 18-membered)heteroaryl. For example, Ar₁₁ and Ar₁₂ each independently may be a substituted or unsubstituted phenyl, a substituted or unsubstituted biphenyl, a substituted or unsubstituted terphenyl, a substituted or unsubstituted naphthyl, a substituted or unsubstituted phenanthrenyl, a substituted or unsubstituted dimethylfluorenyl, a substituted or unsubstituted dibenzofuranyl, a substituted or unsubstituted dibenzothiophenyl, a substituted or unsubstituted carbazolyl, or a substituent represented by the following Formula 2-A. For example, Ar₁₁ and Ar₁₂ each independently may be a substituted or unsubstituted phenyl, a substituted or unsubstituted biphenyl, a substituted or unsubstituted terphenyl, a substituted or unsubstituted naphthyl, or a substituted or unsubstituted phenanthrenyl, or may be represented by the following Formula 2-A, wherein the substituents of the substituted groups may be deuterium, phenyl, biphenyl, terphenyl, naphthyl, or phenanthrenyl.

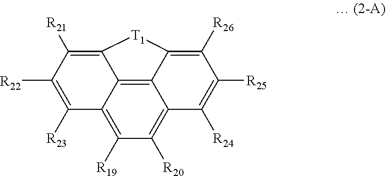

In Formula 2-A,
T₁ represents —O—, —S—, or —CR₂₇R₂₈;
R₁₉ to R₂₈ each independently represent hydrogen, deuterium, a substituted or unsubstituted (C₁-C₃₀)alkyl, a substituted or unsubstituted (C₆-C₃₀)aryl, or a substituted or unsubstituted (3- to 30-membered)heteroaryl; and
any one of R₁₉ to R₂₆ is linked to L₁₁ or L₁₂.

In one embodiment, R₁₉ to R₂₆ each independently may be hydrogen, deuterium, or a substituted or unsubstituted (C₆-C₃₀)aryl, preferably hydrogen, deuterium, or a substituted or unsubstituted (C₆-C₂₅)aryl, more preferably hydrogen, deuterium, or a substituted or unsubstituted (C₆-C₁₈)aryl. For example, R₁₉ to R₂₆ each independently may be hydrogen, deuterium, or a substituted or unsubstituted phenyl.

According to one embodiment, the compound represented by Formula 2 above may be more specifically illustrated by the following compounds, but is not limited thereto.

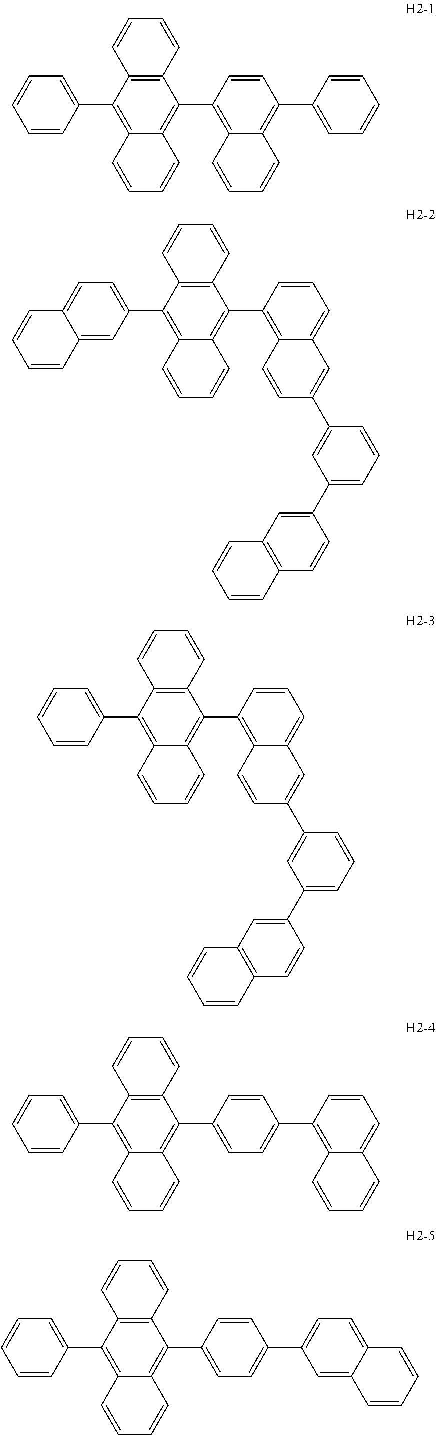

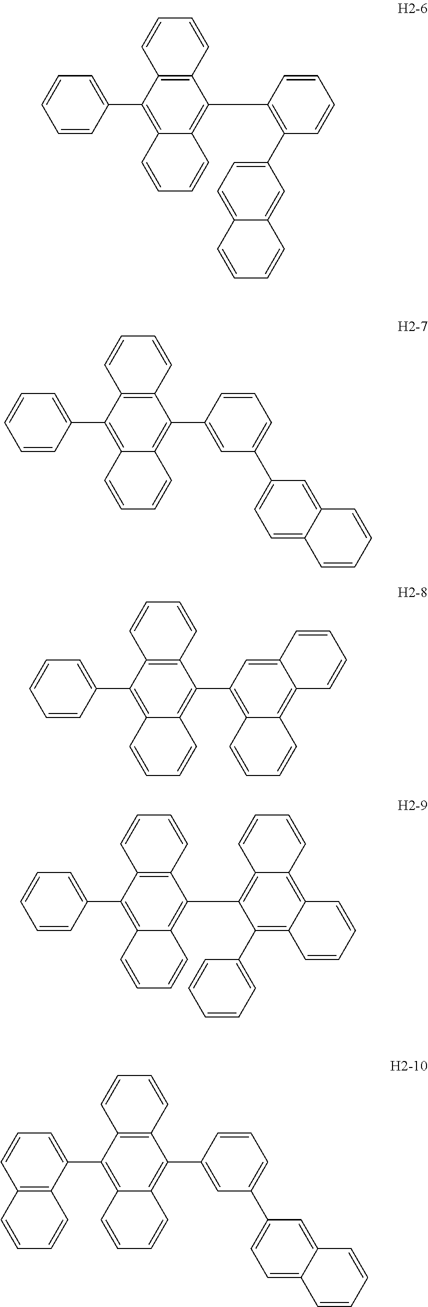

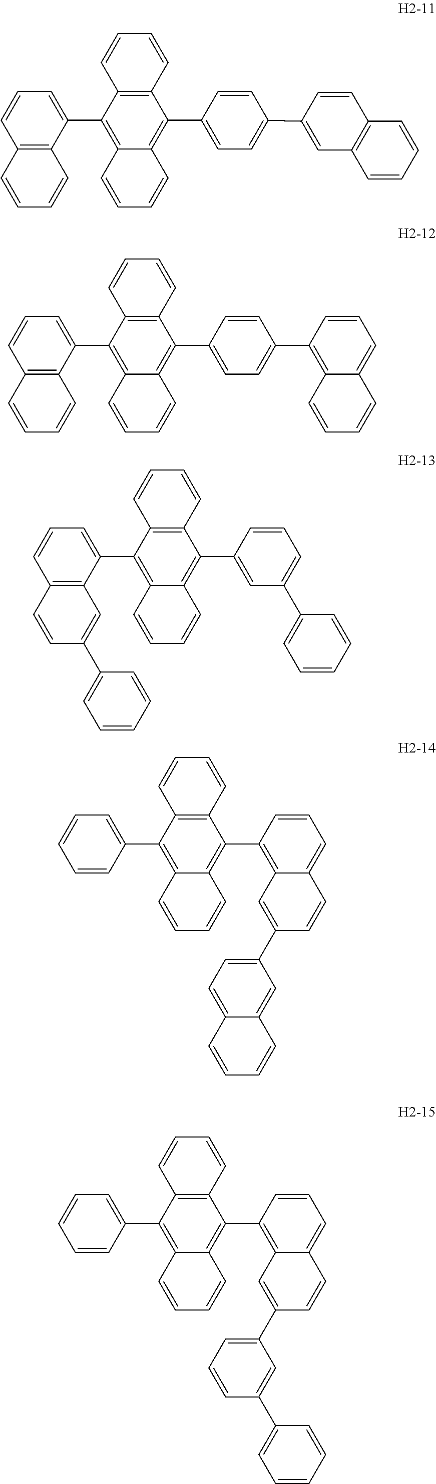

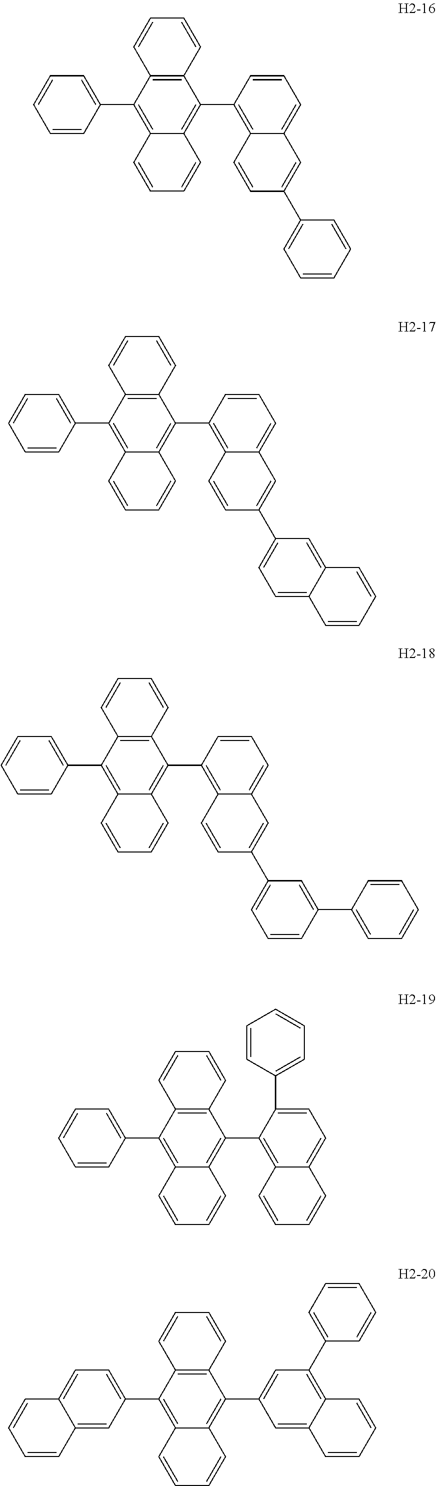

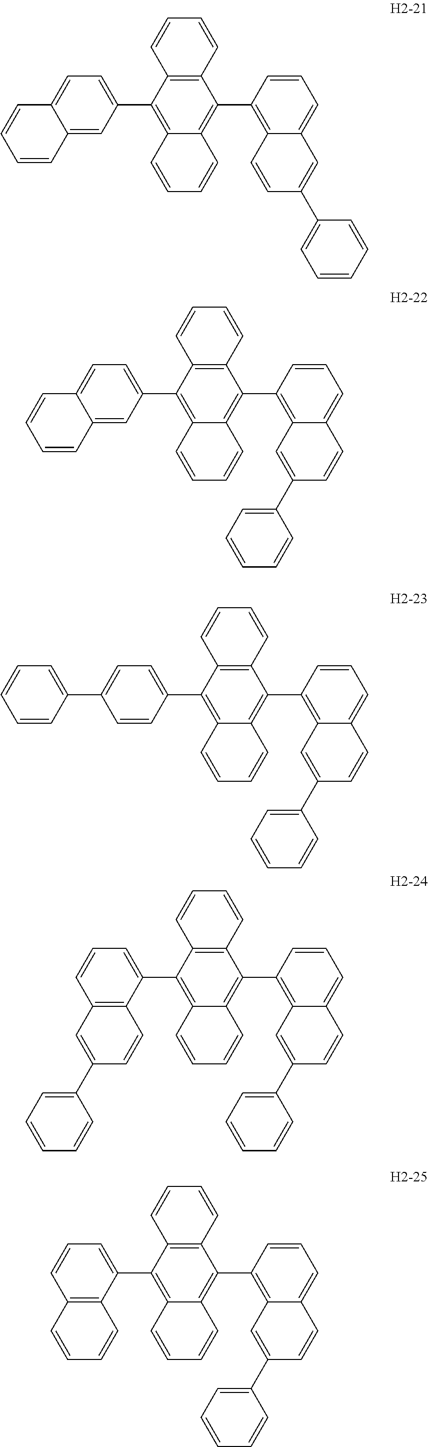

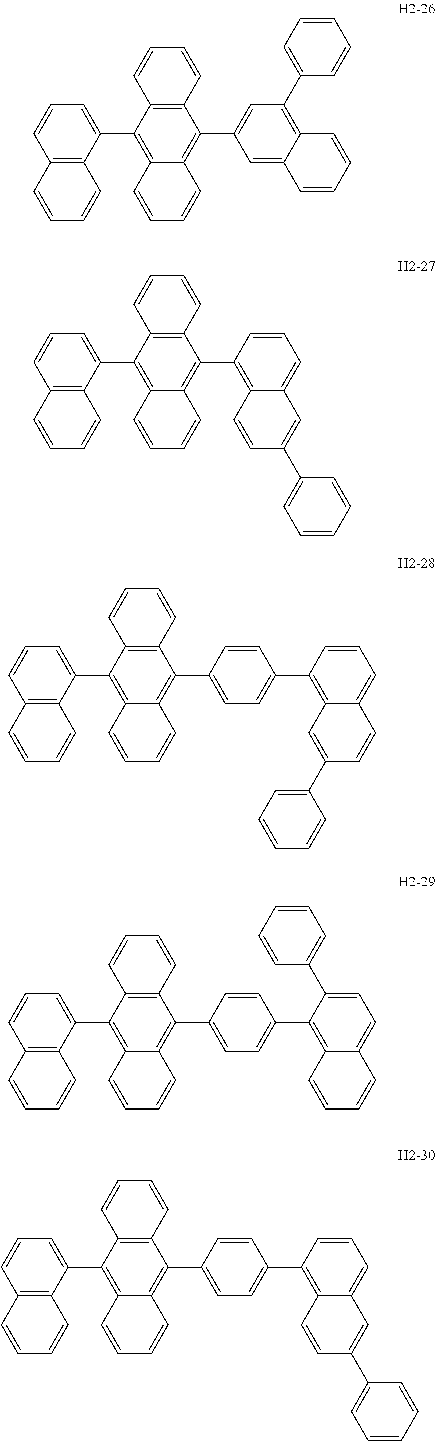

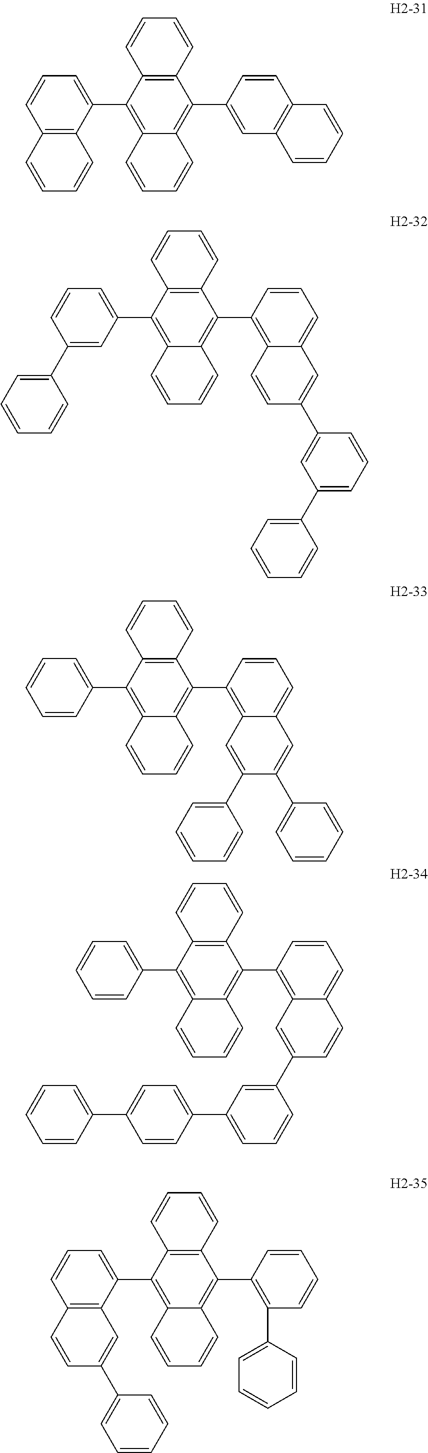

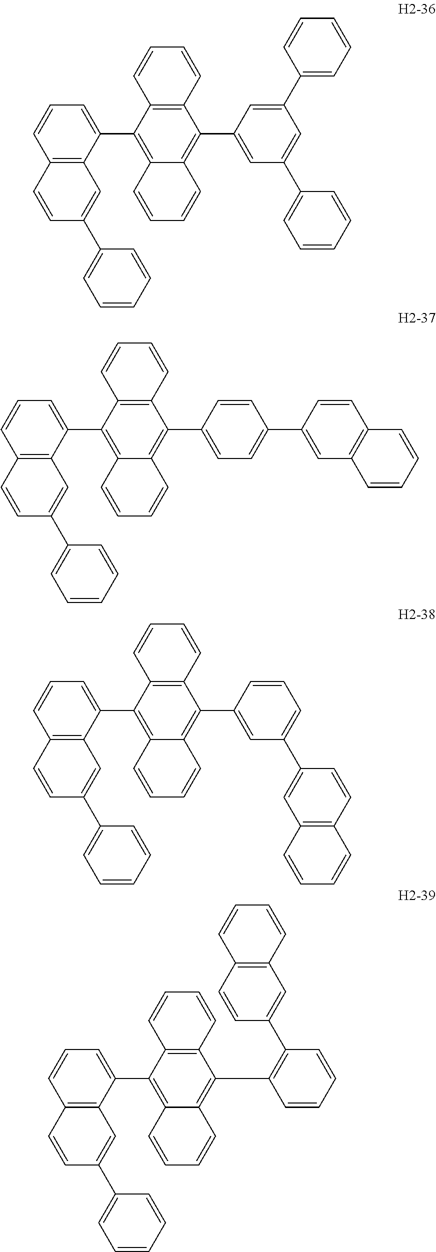

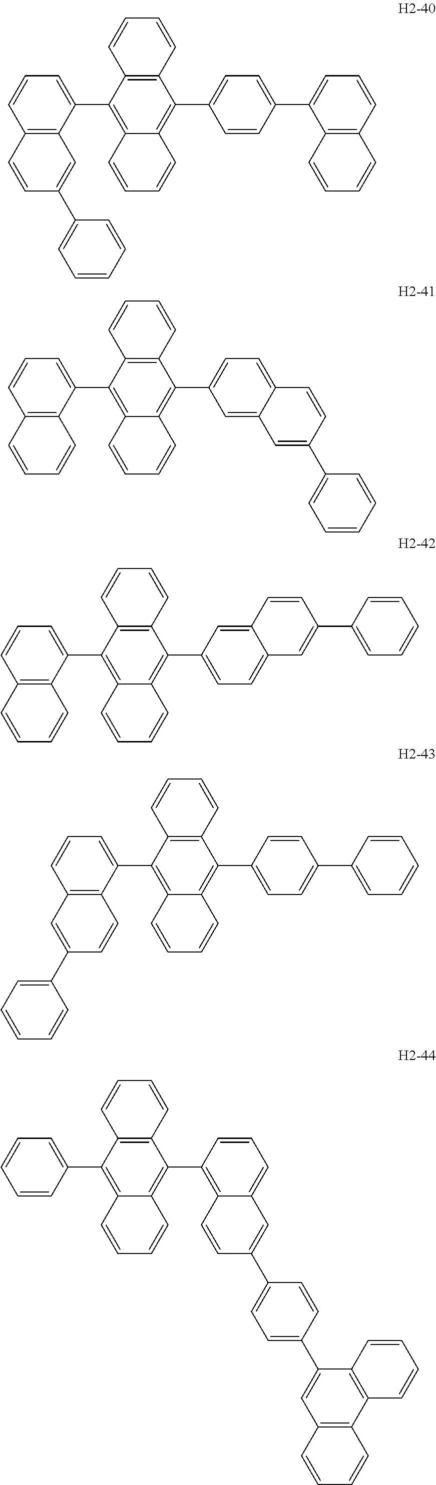

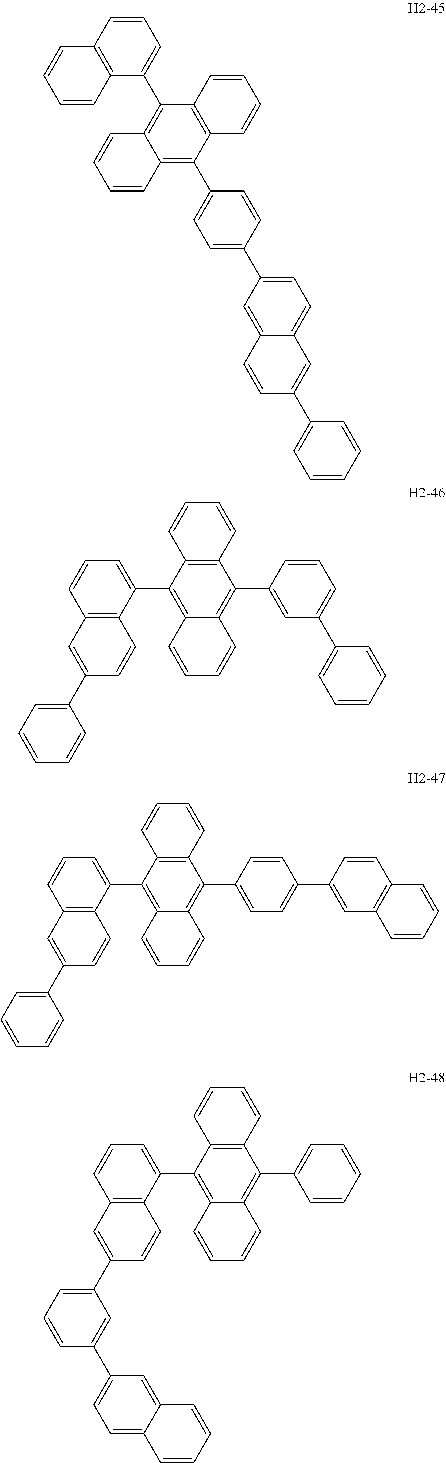

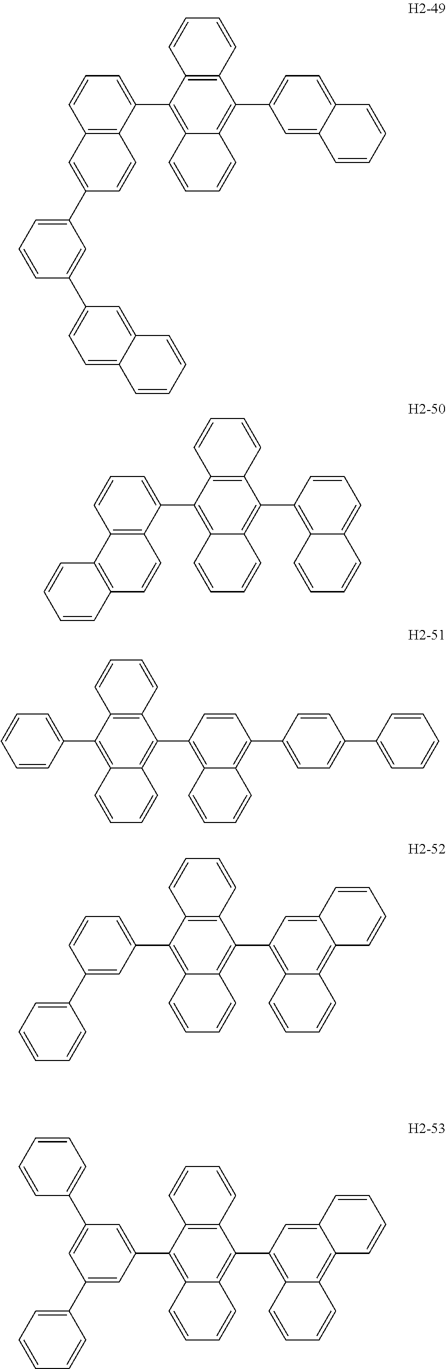

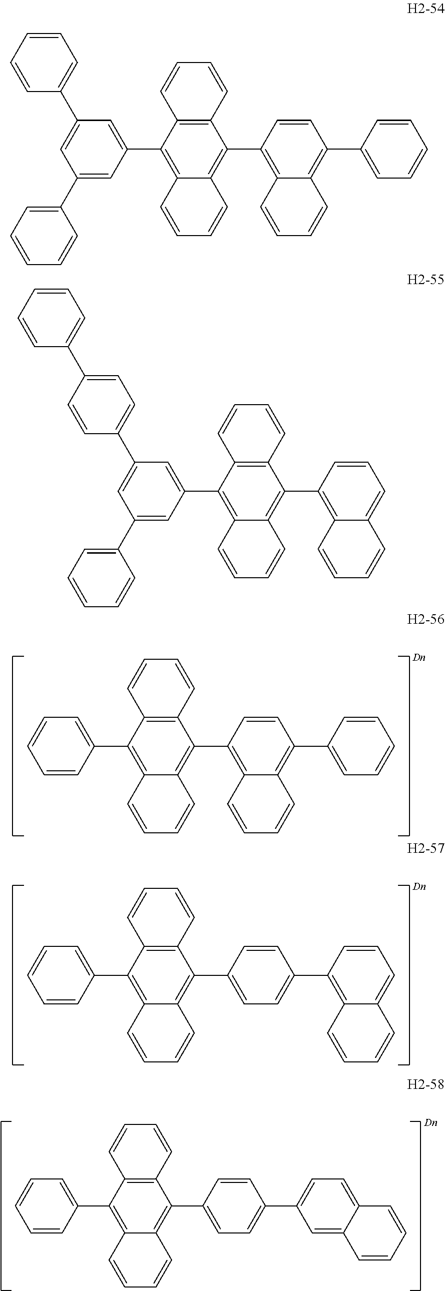

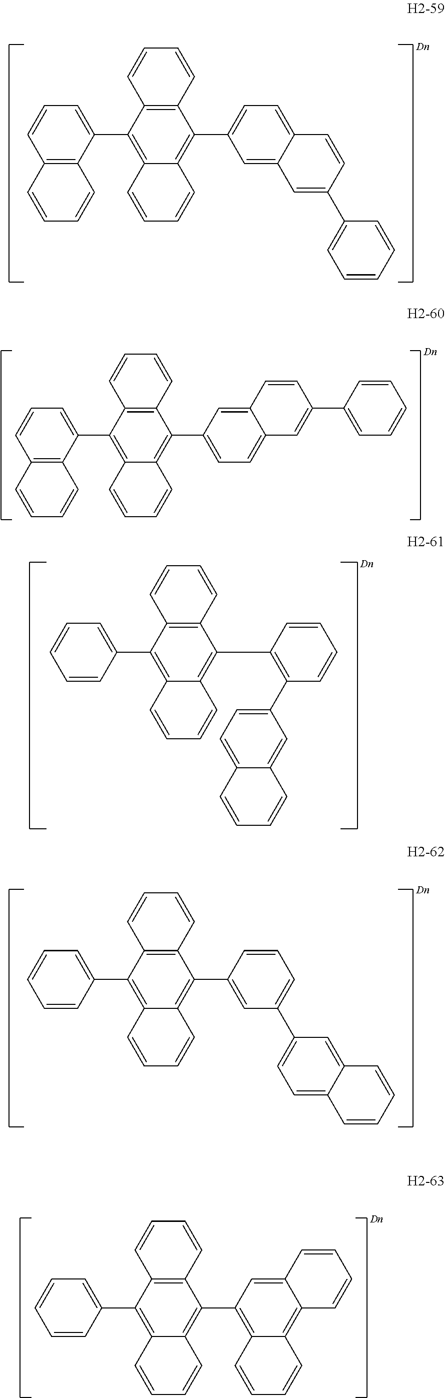

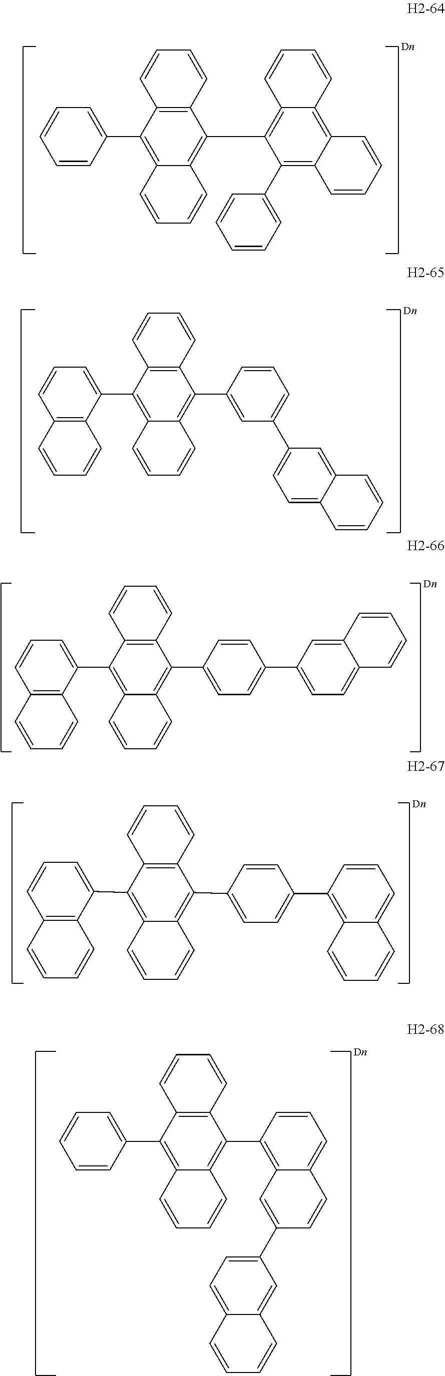

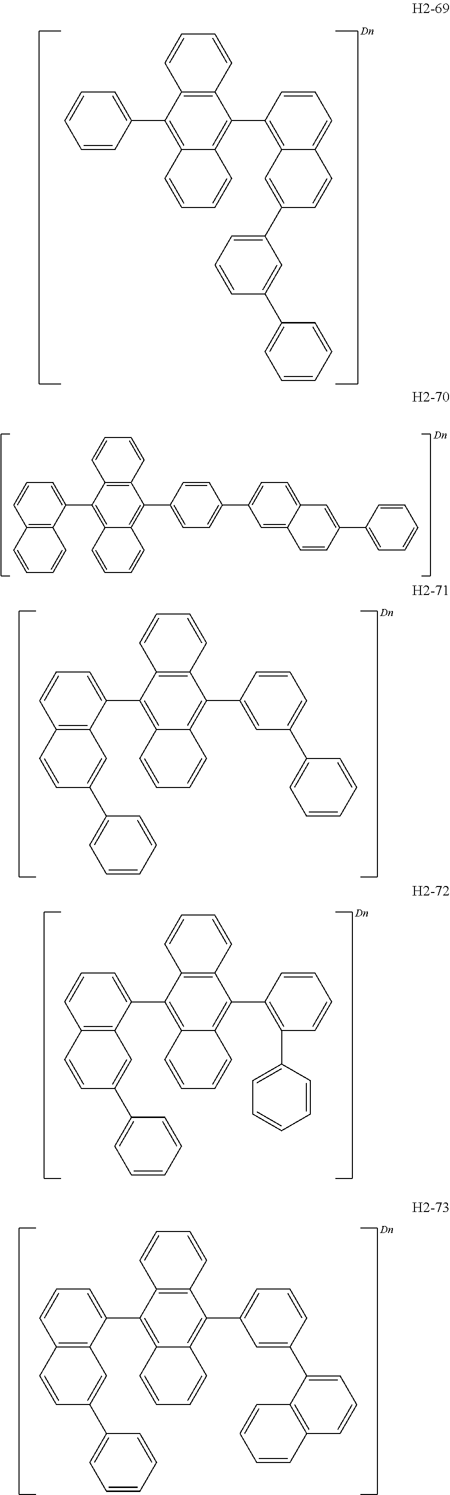

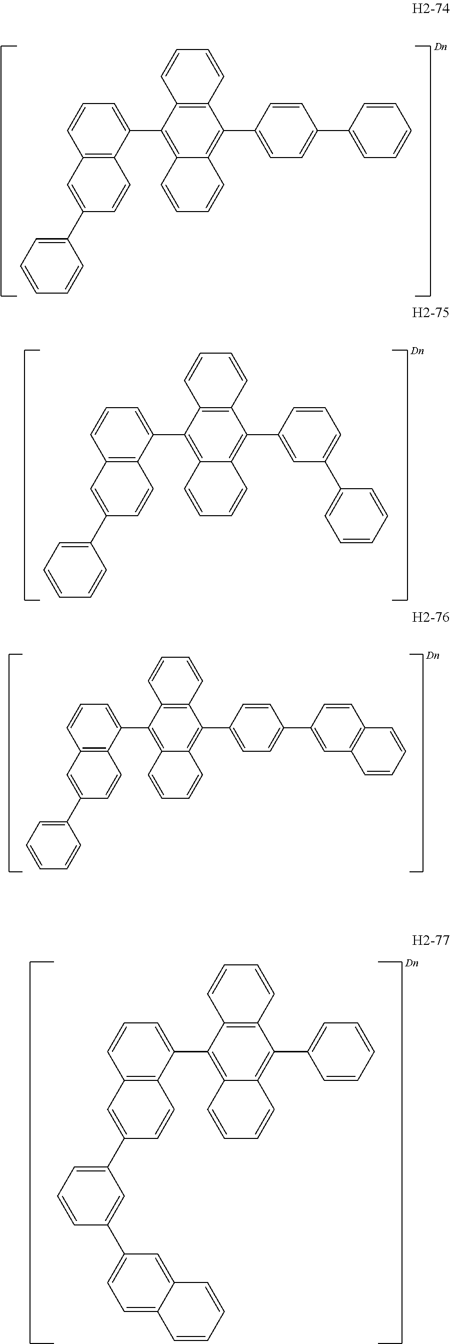

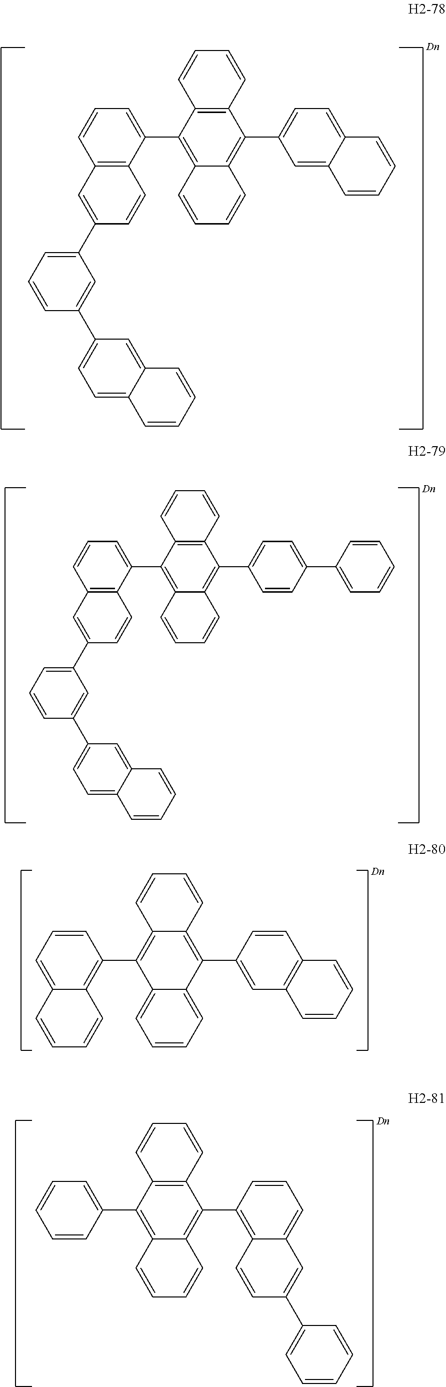

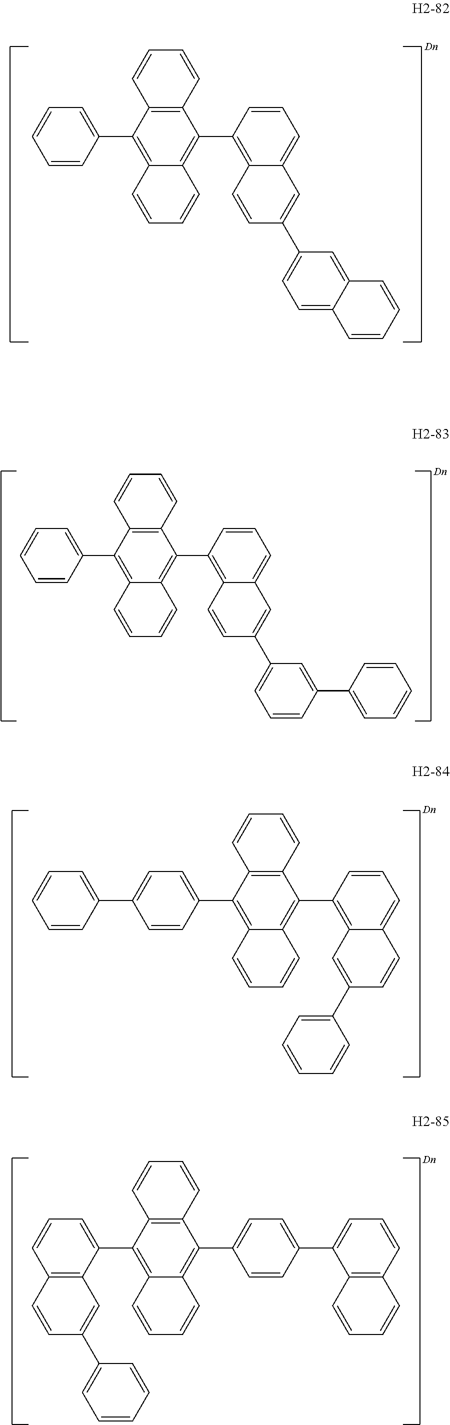

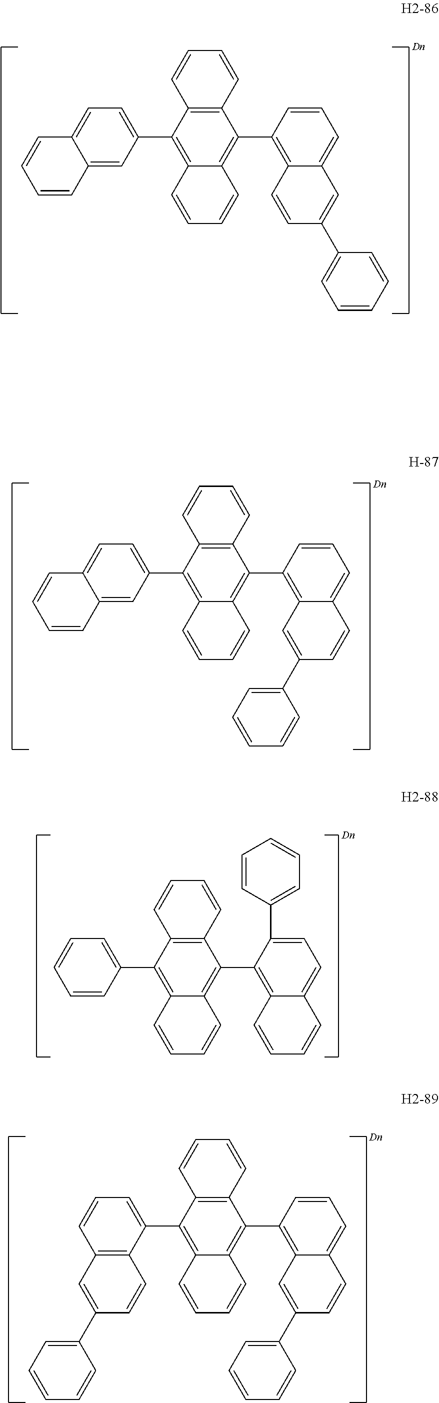

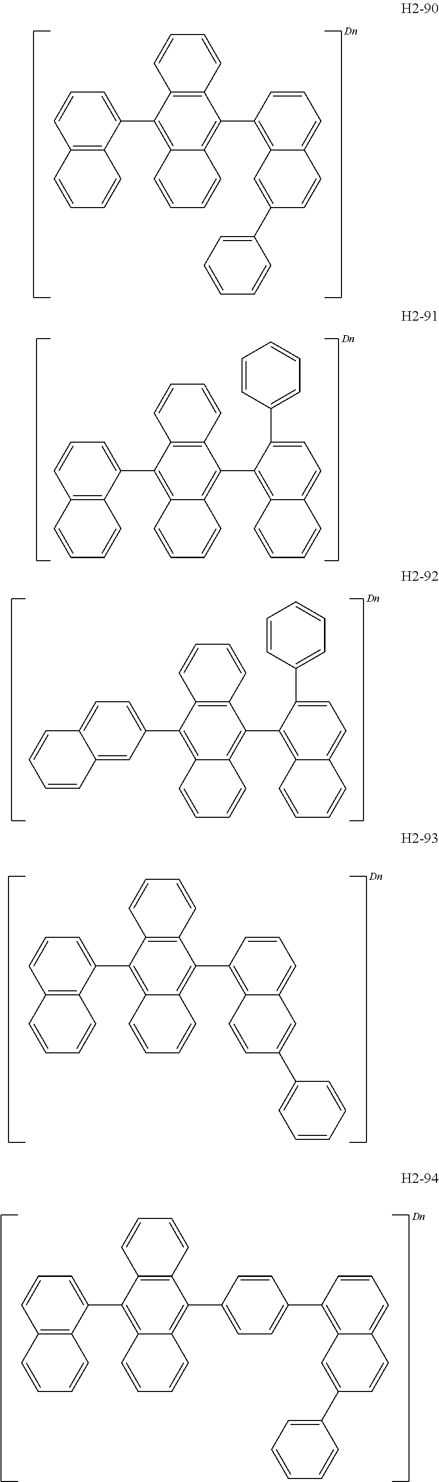

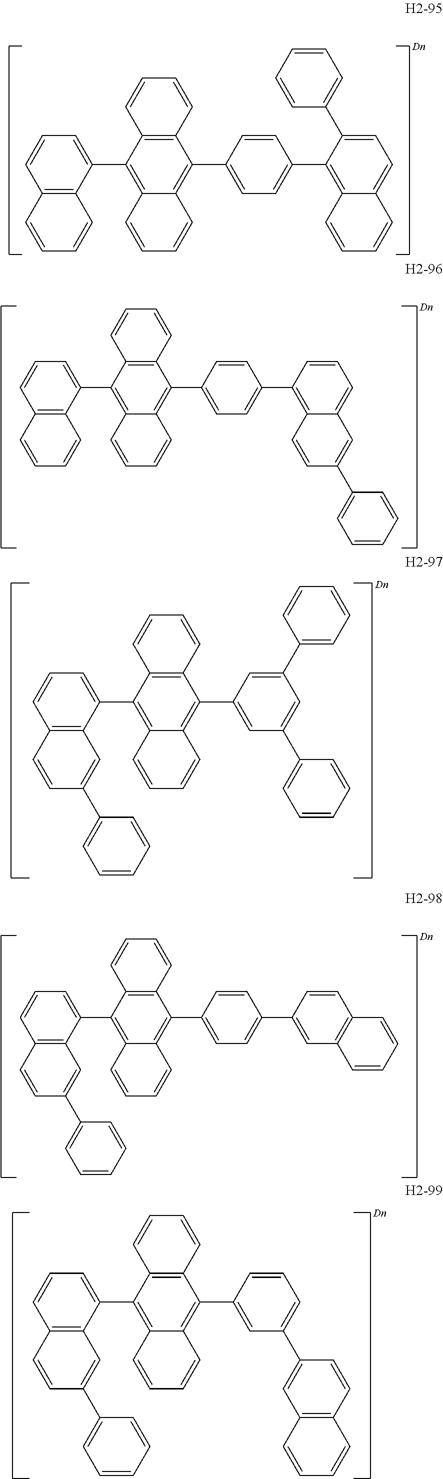

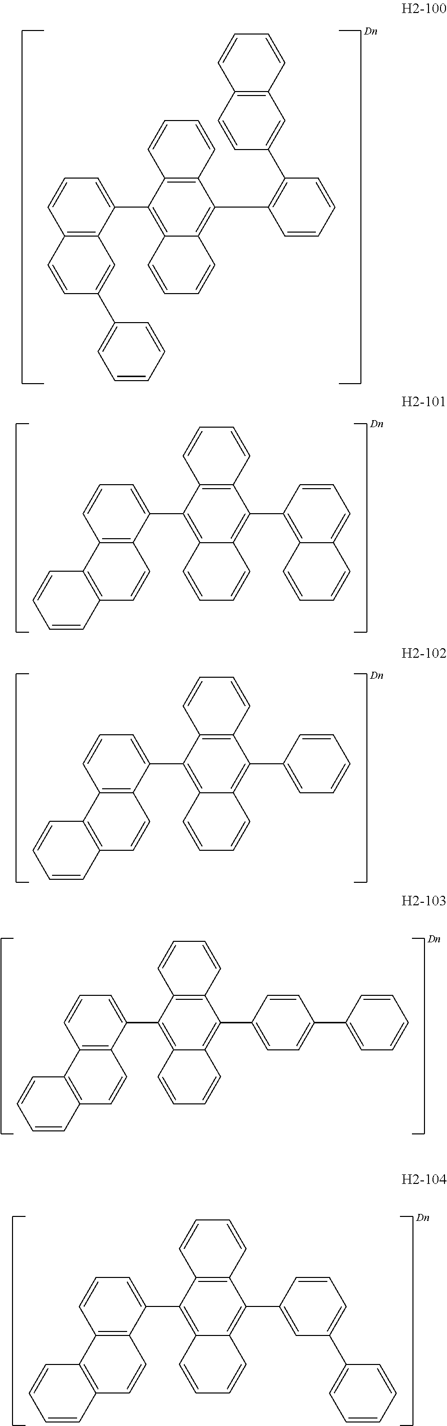

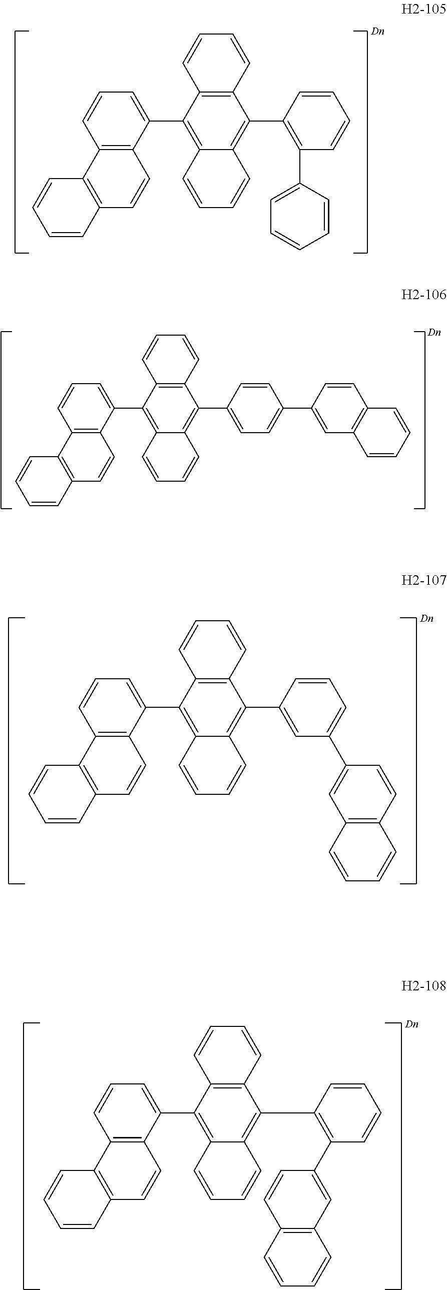

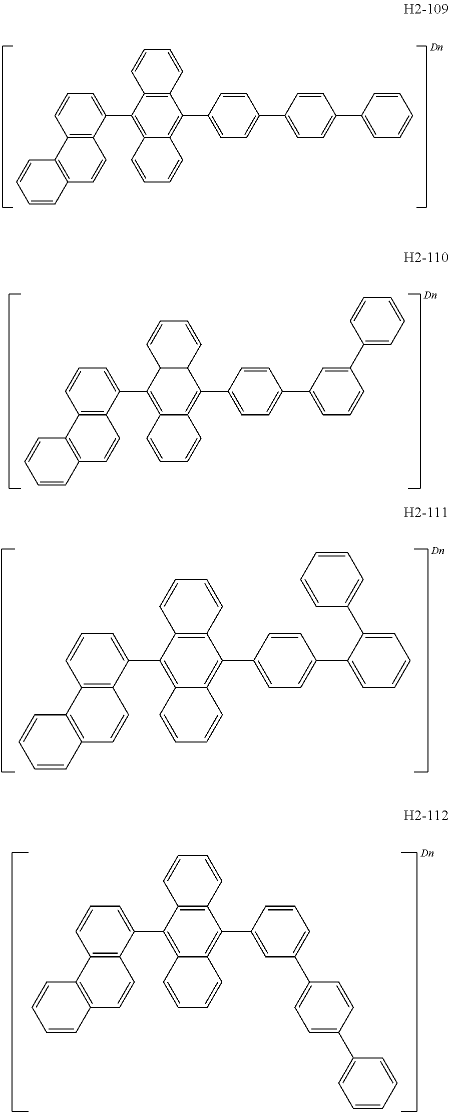

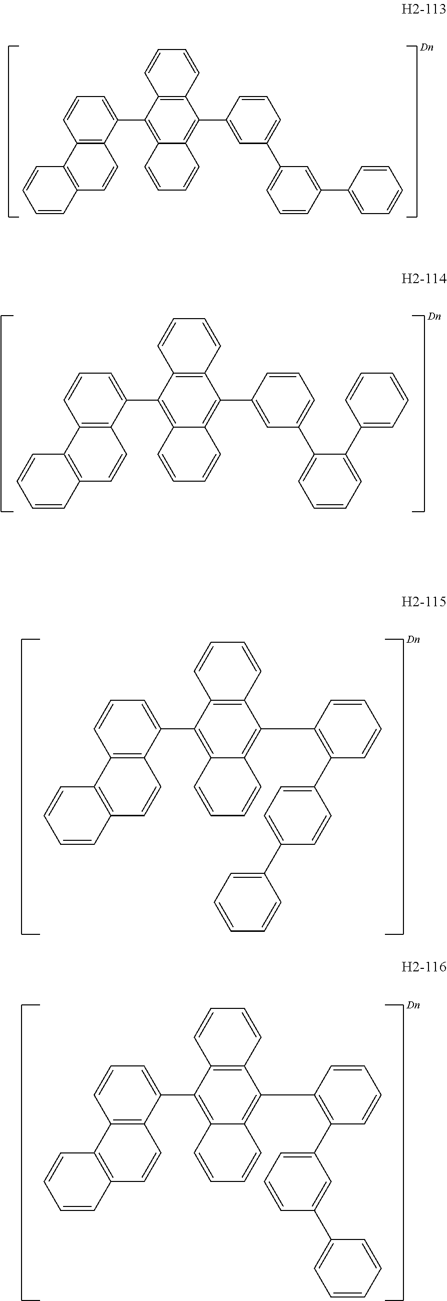

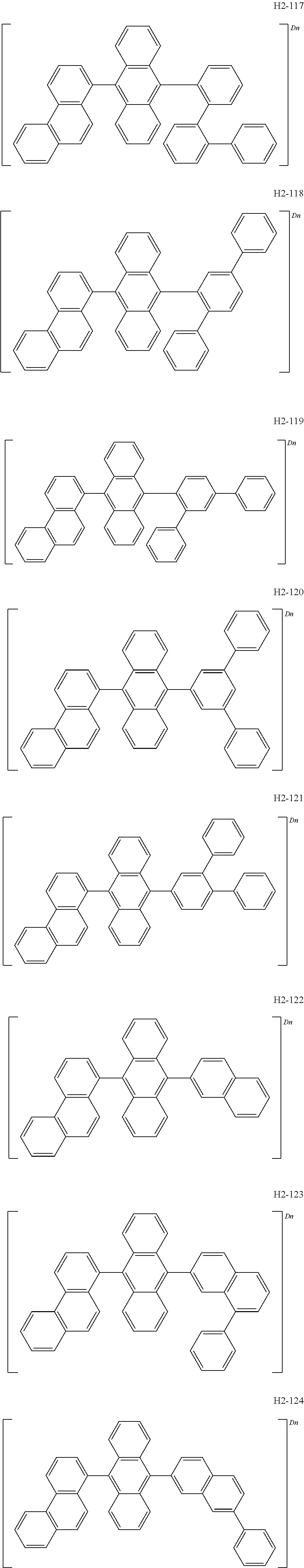

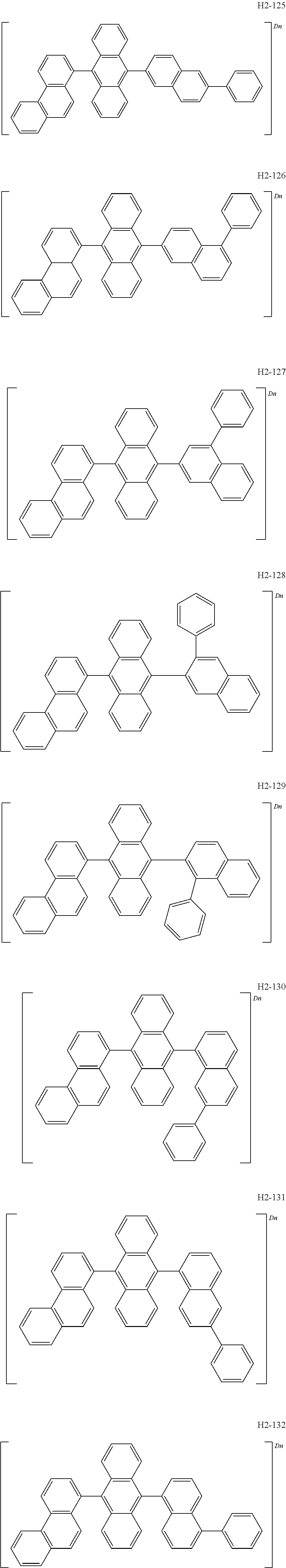

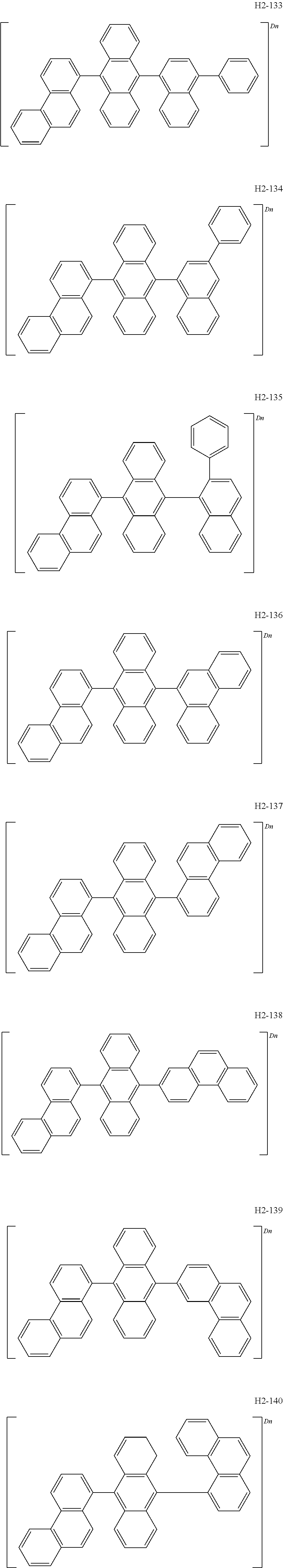

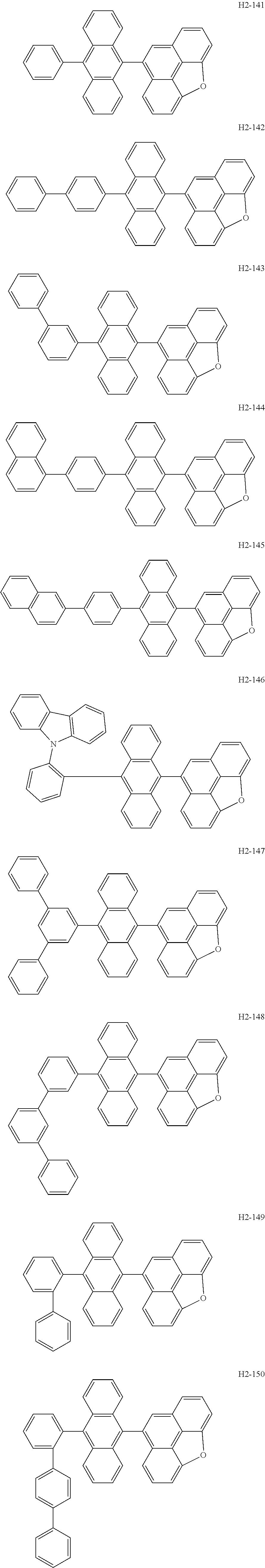

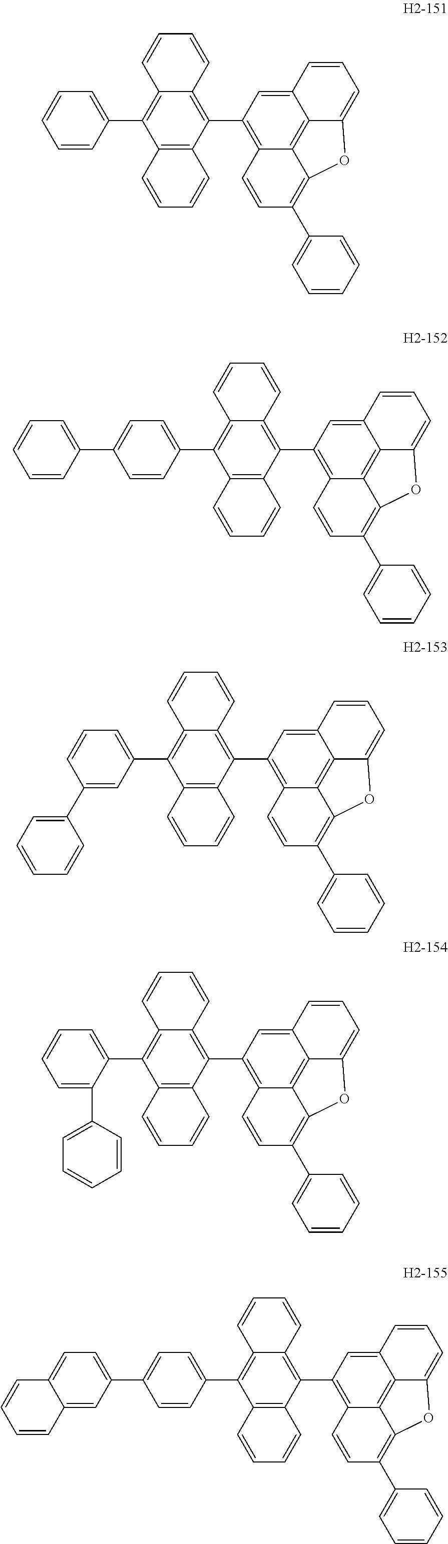 [SMILES: c1ccc(-c2c3ccccc3c(-c3cc4cccc5oc6c(-c7ccccc7)ccc3c6c45)c3ccccc23)cc1 ; c1ccc(-c2ccc(-c3c4ccccc4c(-c4cc5cccc6oc7c(-c8ccccc8)ccc4c7c56)c4ccccc34)cc2)cc1 ; c1ccc(-c2cccc(-c3c4ccccc4c(-c4cc5cccc6oc7c(-c8ccccc8)ccc4c7c56)c4ccccc34)c2)cc1 ; c1ccc(-c2ccccc2-c2c3ccccc3c(-c3cc4cccc5oc6c(-c7ccccc7)ccc3c6c45)c3ccccc23)cc1 ; c1ccc(-c2ccc3c(-c4c5ccccc5c(-c5ccc(-c6ccc7ccccc7c6)cc5)c5ccccc45)cc4cccc5oc2c3c45)cc1]

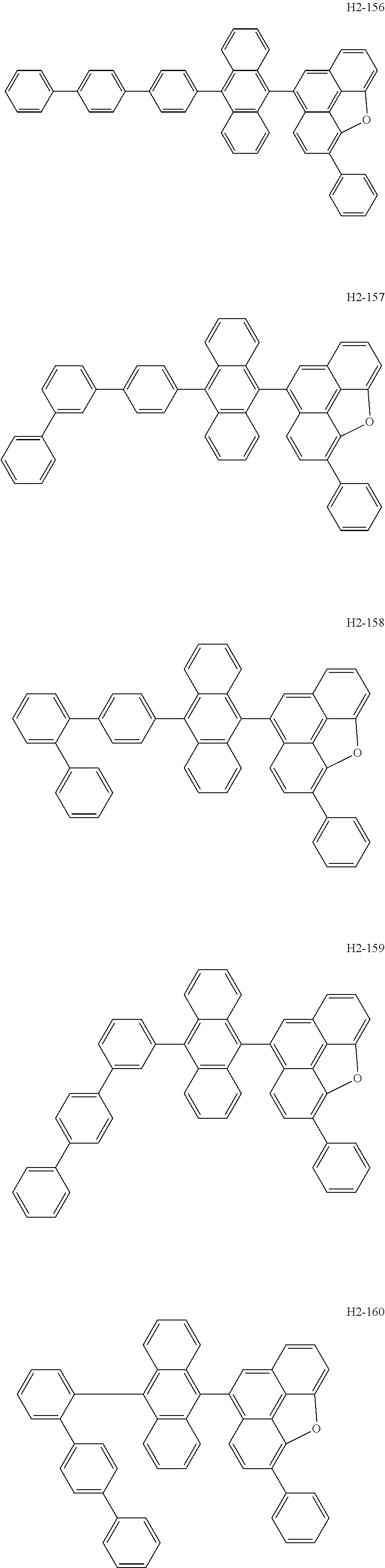 [SMILES: c1ccc(-c2ccc(-c3ccc(-c4c5ccccc5c(-c5cc6cccc7oc8c(-c9ccccc9)ccc5c8c67)c5ccccc45)cc3)cc2)cc1 ; c1ccc(-c2cccc(-c3ccc(-c4c5ccccc5c(-c5cc6cccc7oc8c(-c9ccccc9)ccc5c8c67)c5ccccc45)cc3)c2)cc1 ; c1ccc(-c2ccccc2-c2ccc(-c3c4ccccc4c(-c4cc5cccc6oc7c(-c8ccccc8)ccc4c7c56)c4ccccc34)cc2)cc1 ; c1ccc(-c2ccc(-c3cccc(-c4c5ccccc5c(-c5cc6cccc7oc8c(-c9ccccc9)ccc5c8c67)c5ccccc45)c3)cc2)cc1 ; c1ccc(-c2ccc(-c3ccccc3-c3c4ccccc4c(-c4cc5cccc6oc7c(-c8ccccc8)ccc4c7c56)c4ccccc34)cc2)cc1]

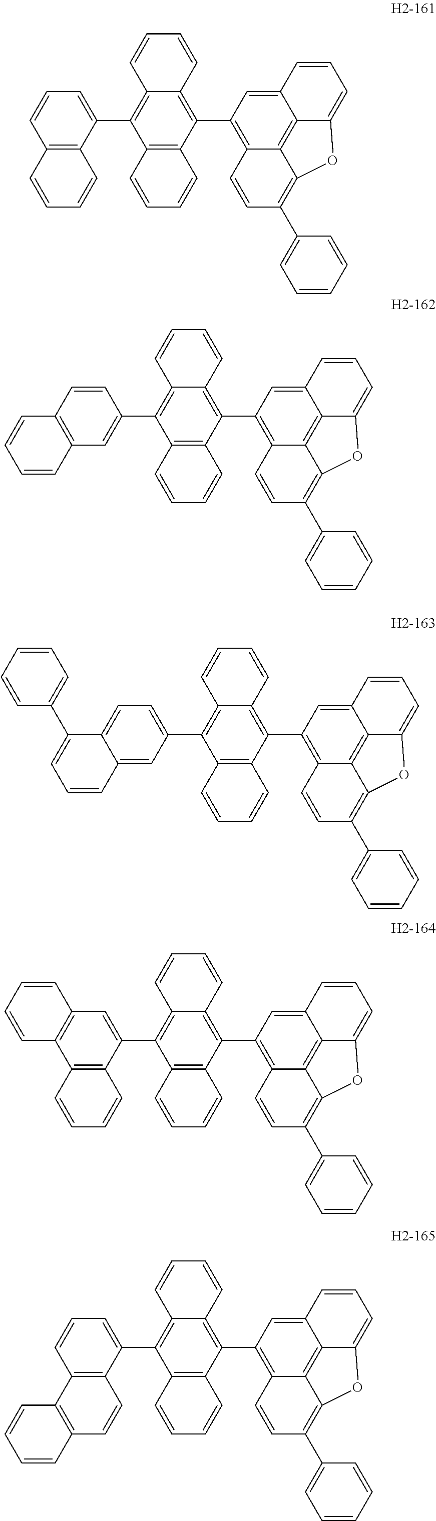 [SMILES: c1ccc(-c2ccc3c(-c4c5ccccc5c(-c5cccc6ccccc56)c5ccccc45)cc4cccc5oc2c3c45)cc1 ; c1ccc(-c2ccc3c(-c4c5ccccc5c(-c5ccc6ccccc6c5)c5ccccc45)cc4cccc5oc2c3c45)cc1 ; c1ccc(-c2cccc3cc(-c4c5ccccc5c(-c5cc6cccc7oc8c(-c9ccccc9)ccc5c8c67)c5ccccc45)ccc23)cc1 ; c1ccc(-c2ccc3c(-c4c5ccccc5c(-c5cc6ccccc6c6ccccc56)c5ccccc45)cc4cccc5oc2c3c45)cc1 ; c1ccc(-c2ccc3c(-c4c5ccccc5c(-c5cccc6c5ccc5ccccc56)c5ccccc45)cc4cccc5oc2c3c45)cc1]

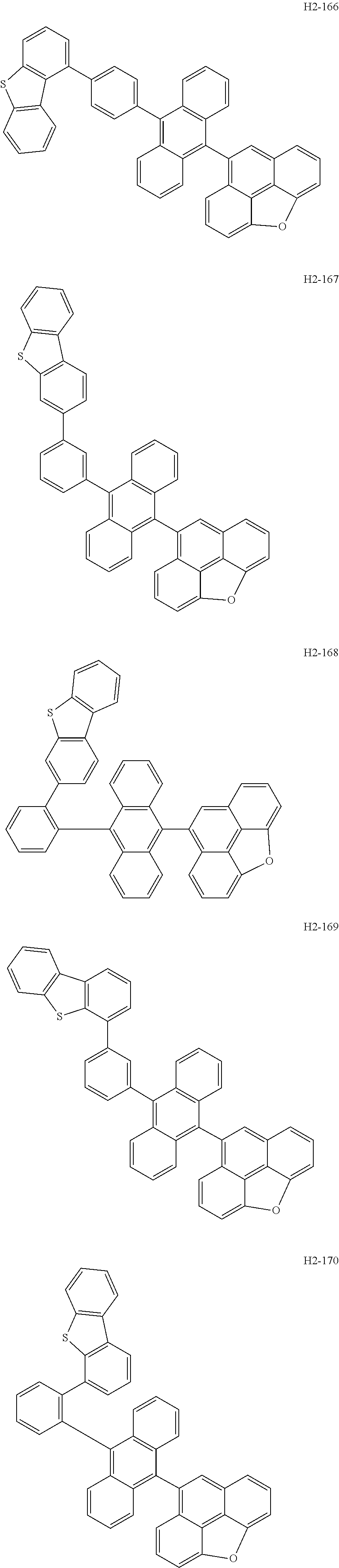 [SMILES: c1cc2cc(-c3c4ccccc4c(-c4ccc(-c5cccc6sc7ccccc7c56)cc4)c4ccccc34)c3cccc4oc(c1)c2c43 ; c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1 ; c1cc(-c2c3ccccc3c(-c3cc4cccc5oc6cccc3c6c45)c3ccccc23)cc(-c2cccc3c2sc2ccccc23)c1 ; c1ccc(-c2cccc3c2sc2ccccc23)c(-c2c3ccccc3c(-c3cc4cccc5oc6cccc3c6c45)c3ccccc23)c1]

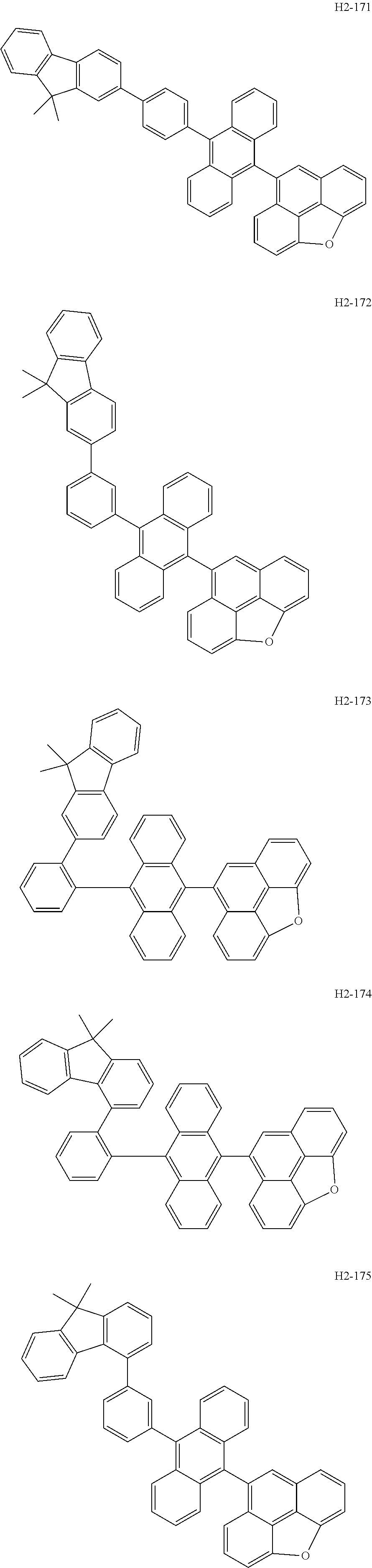 [SMILES: CC1(C)c2ccccc2-c2ccc(-c3ccc(-c4c5ccccc5c(-c5cc6cccc7oc8cccc5c8c67)c5ccccc45)cc3)cc21 ; CC1(C)c2ccccc2-c2ccc(-c3cccc(-c4c5ccccc5c(-c5cc6cccc7oc8cccc5c8c67)c5ccccc45)c3)cc21 ; CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21 ; CC1(C)c2ccccc2-c2c(-c3cccc(-c4c5ccccc5c(-c5cc6cccc7oc8cccc5c8c67)c5ccccc45)c3)cccc21]

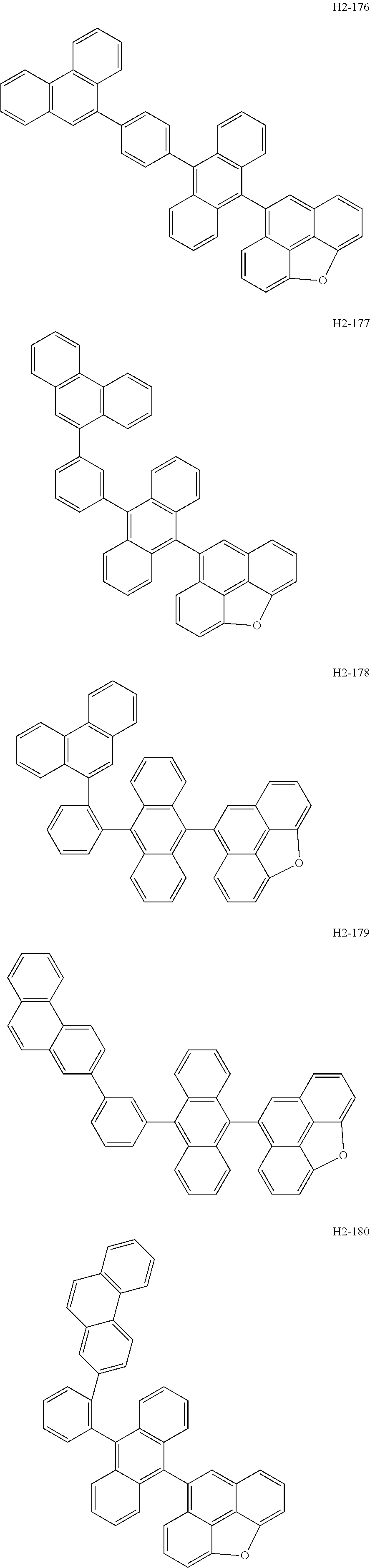 [SMILES: c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1 ; c1ccc(-c2cc3ccccc3c3ccccc23)c(-c2c3ccccc3c(-c3cc4cccc5oc6cccc3c6c45)c3ccccc23)c1 ; c1cc(-c2ccc3c(ccc4ccccc43)c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(ccc4ccccc43)c2)c1]

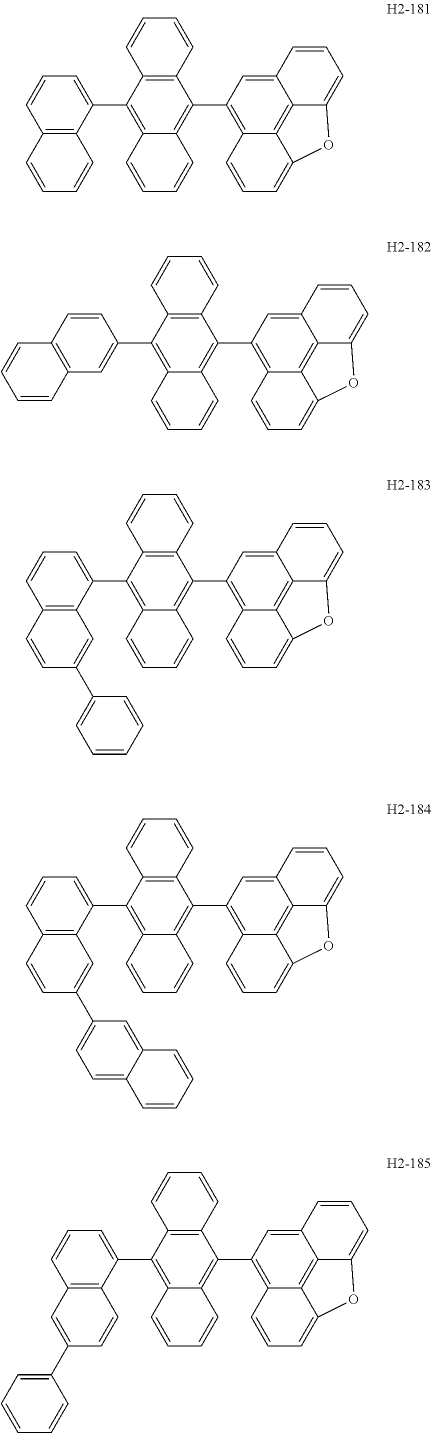 [SMILES: c1ccc2c(-c3c4ccccc4c(-c4cc5cccc6oc7cccc4c7c56)c4ccccc34)cccc2c1 ; c1ccc2cc(-c3c4ccccc4c(-c4cc5cccc6oc7cccc4c7c56)c4ccccc34)ccc2c1 ; c1ccc(-c2ccc3cccc(-c4c5ccccc5c(-c5cc6cccc7oc8cccc5c8c67)c5ccccc45)c3c2)cc1 ; c1ccc2cc(-c3ccc4cccc(-c5c6ccccc6c(-c6cc7cccc8oc9cccc6c9c78)c6ccccc56)c4c3)ccc2c1 ; c1ccc(-c2ccc3c(-c4c5ccccc5c(-c5cc6cccc7oc8cccc5c8c67)c5ccccc45)cccc3c2)cc1]

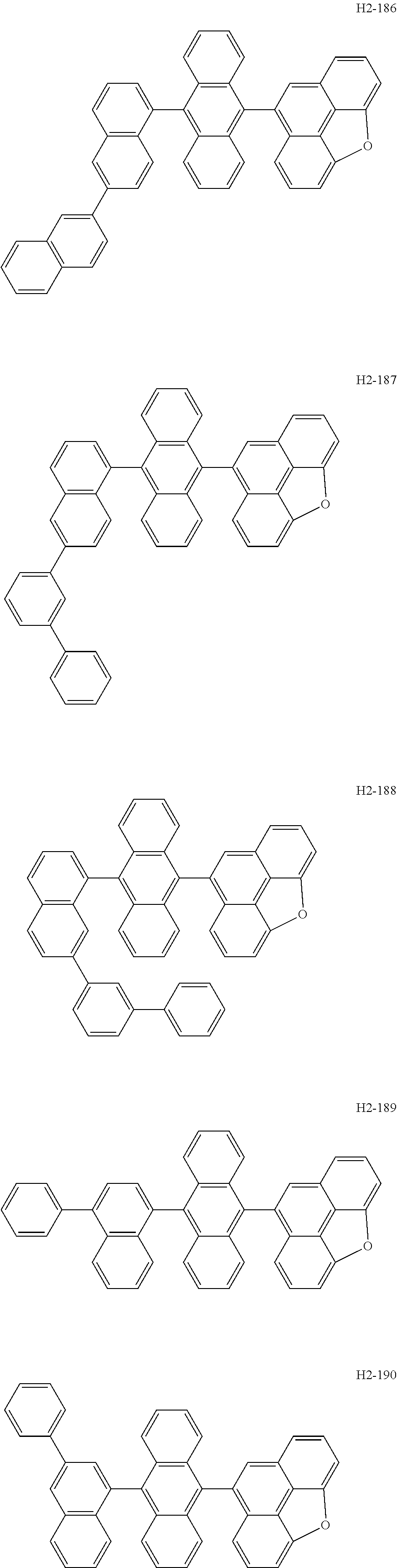 [SMILES: c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1 ; c1ccc(-c2ccc(-c3c4ccccc4c(-c4cc5cccc6oc7cccc4c7c56)c4ccccc34)c3ccccc23)cc1 ; c1ccc(-c2cc(-c3c4ccccc4c(-c4cc5cccc6oc7cccc4c7c56)c4ccccc34)c3ccccc3c2)cc1]

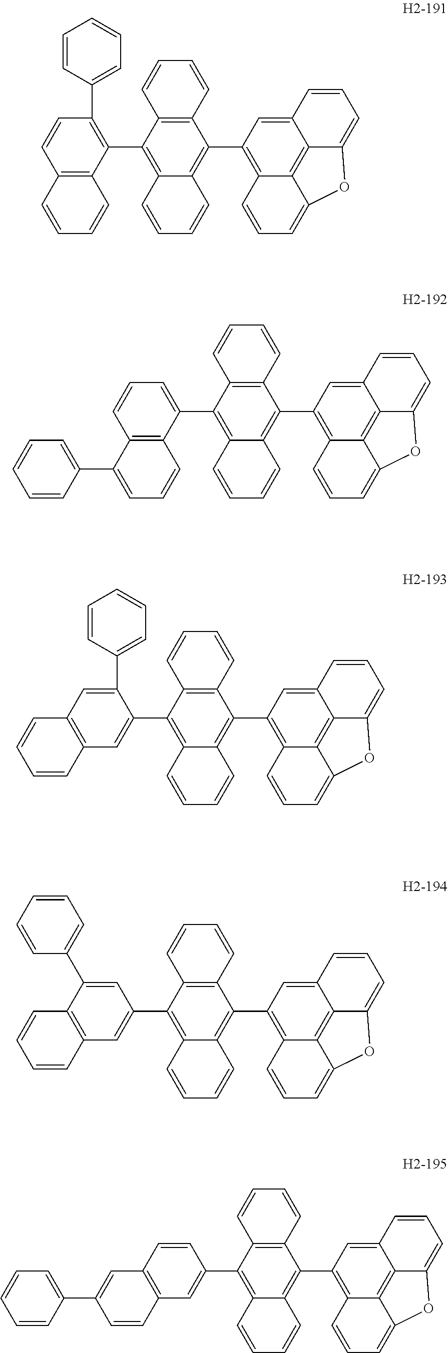 [SMILES: c1ccc(-c2ccc3ccccc3c2-c2c3ccccc3c(-c3cc4cccc5oc6cccc3c6c45)c3ccccc23)cc1 ; c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)cc3ccccc23)cc1 ; c1ccc(-c2ccc3cc(-c4c5ccccc5c(-c5cc6cccc7oc8cccc5c8c67)c5ccccc45)ccc3c2)cc1]

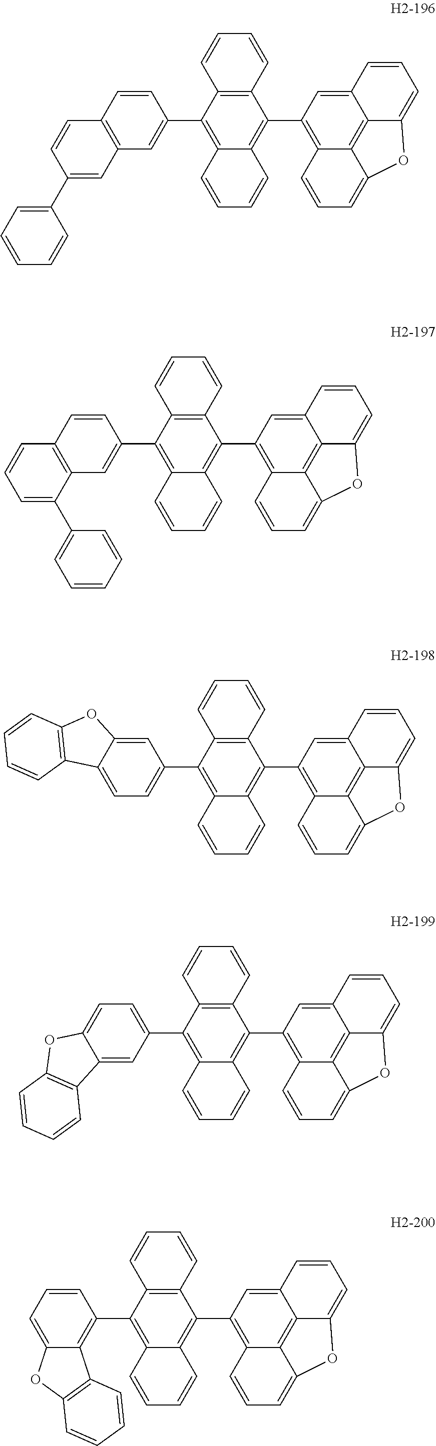 [SMILES: c1ccc(-c2ccc3ccc(-c4c5ccccc5c(-c5cc6cccc7oc8cccc5c8c67)c5ccccc45)cc3c2)cc1 ; c1ccc(-c2cccc3ccc(-c4c5ccccc5c(-c5cc6cccc7oc8cccc5c8c67)c5ccccc45)cc23)cc1 ; c1ccc2c(c1)oc1cc(-c3c4ccccc4c(-c4cc5cccc6oc7cccc4c7c56)c4ccccc34)ccc12 ; c1ccc2c(c1)oc1ccc(-c3c4ccccc4c(-c4cc5cccc6oc7cccc4c7c56)c4ccccc34)cc12 ; c1ccc2c(c1)oc1cccc(-c3c4ccccc4c(-c4cc5cccc6oc7cccc4c7c56)c4ccccc34)c12]

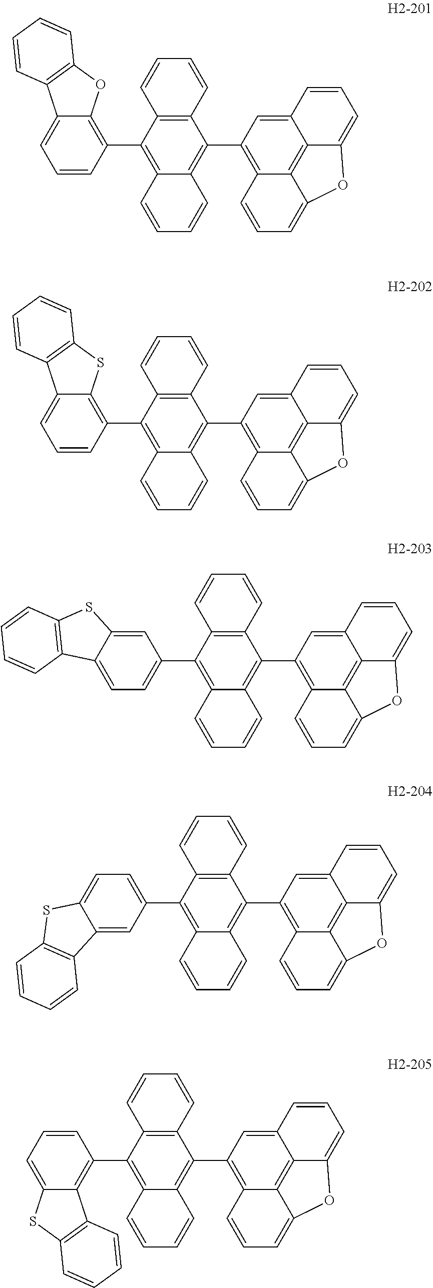 [SMILES: c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43 ; c1cc2cc(-c3c4ccccc4c(-c4ccc5sc6ccccc6c5c4)c4ccccc34)c3cccc4oc(c1)c2c43 ; c1cc2cc(-c3c4ccccc4c(-c4cccc5sc6ccccc6c45)c4ccccc34)c3cccc4oc(c1)c2c43]

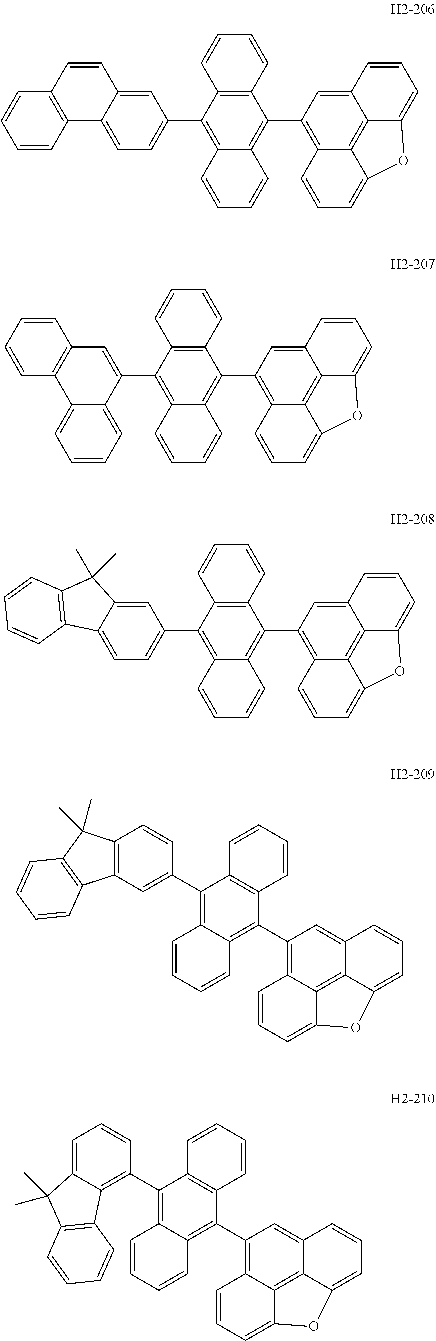 [SMILES: c1ccc2c(c1)ccc1cc(-c3c4ccccc4c(-c4cc5cccc6oc7cccc4c7c56)c4ccccc34)ccc12 ; c1ccc2c(c1)cc(-c1c3ccccc3c(-c3cc4cccc5oc6cccc3c6c45)c3ccccc13)c1ccccc12 ; CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21 ; CC1(C)c2ccccc2-c2c(-c3c4ccccc4c(-c4cc5cccc6oc7cccc4c7c56)c4ccccc34)cccc21]

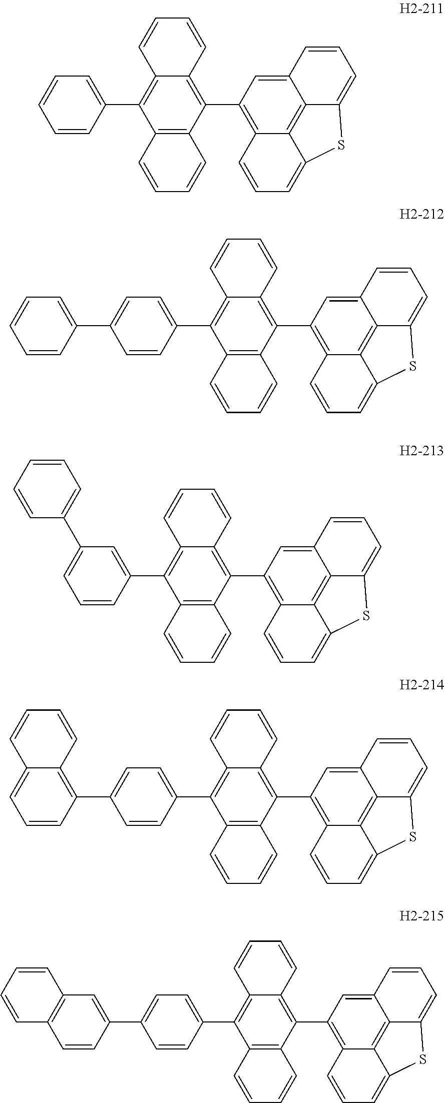 [SMILES: c1ccc(-c2c3ccccc3c(-c3cc4cccc5sc6cccc3c6c45)c3ccccc23)cc1 ; c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1 ; c1ccc2c(-c3ccc(-c4c5ccccc5c(-c5cc6cccc7sc8cccc5c8c67)c5ccccc45)cc3)cccc2c1 ; c1ccc2cc(-c3ccc(-c4c5ccccc5c(-c5cc6cccc7sc8cccc5c8c67)c5ccccc45)cc3)ccc2c1]

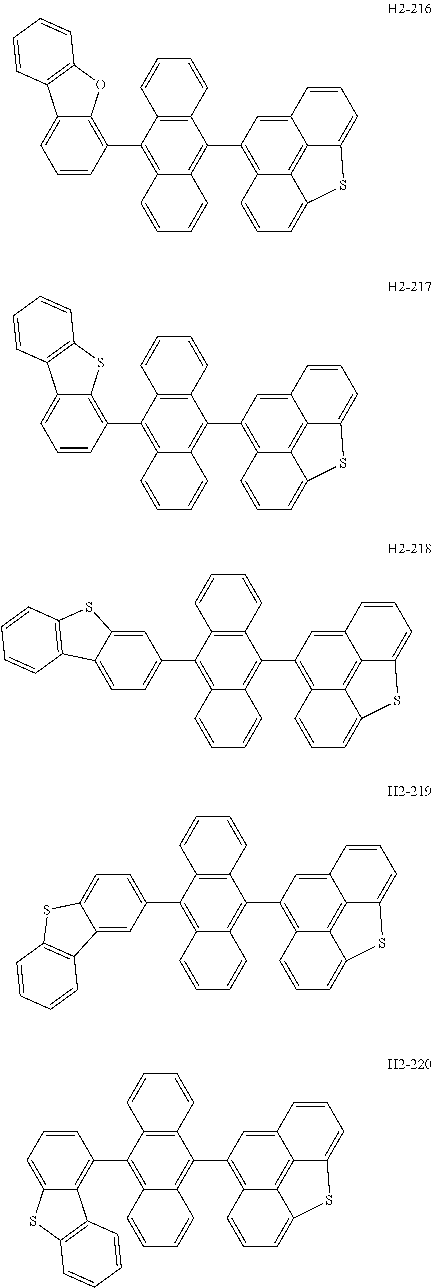 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12]

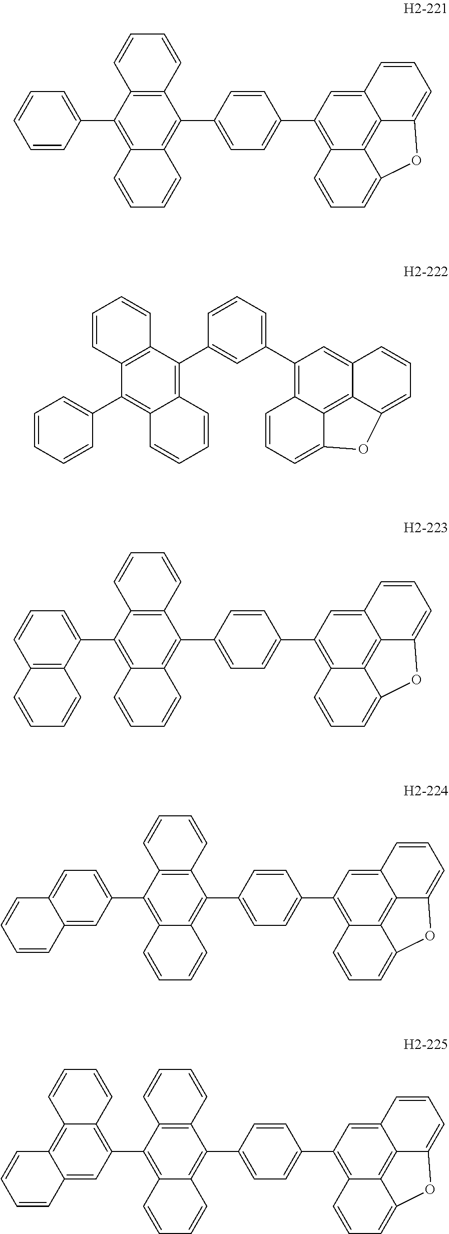 [SMILES: c1ccc(-c2c3ccccc3c(-c3ccc(-c4cc5cccc6oc7cccc4c7c56)cc3)c3ccccc23)cc1 ; c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1 ; c1ccc2cc(-c3c4ccccc4c(-c4ccc(-c5cc6cccc7oc8cccc5c8c67)cc4)c4ccccc34)ccc2c1 ; c1ccc2c(c1)cc(-c1c3ccccc3c(-c3ccc(-c4cc5cccc6oc7cccc4c7c56)cc3)c3ccccc13)c1ccccc12]

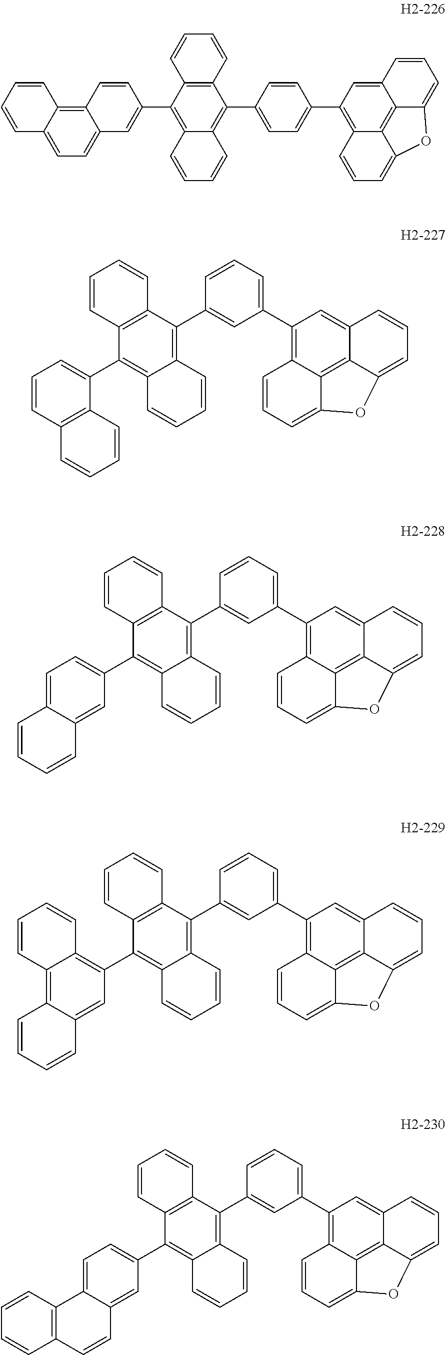 [SMILES: c1ccc2c(c1)ccc1cc(-c3c4ccccc4c(-c4ccc(-c5cc6cccc7oc8cccc5c8c67)cc4)c4ccccc34)ccc12 ; c1cc(-c2c3ccccc3c(-c3cccc4ccccc34)c3ccccc23)cc(-c2cc3cccc4oc5cccc2c5c34)c1 ; c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1 ; c1cc(-c2c3ccccc3c(-c3ccc4c(ccc5ccccc54)c3)c3ccccc23)cc(-c2cc3cccc4oc5cccc2c5c34)c1]

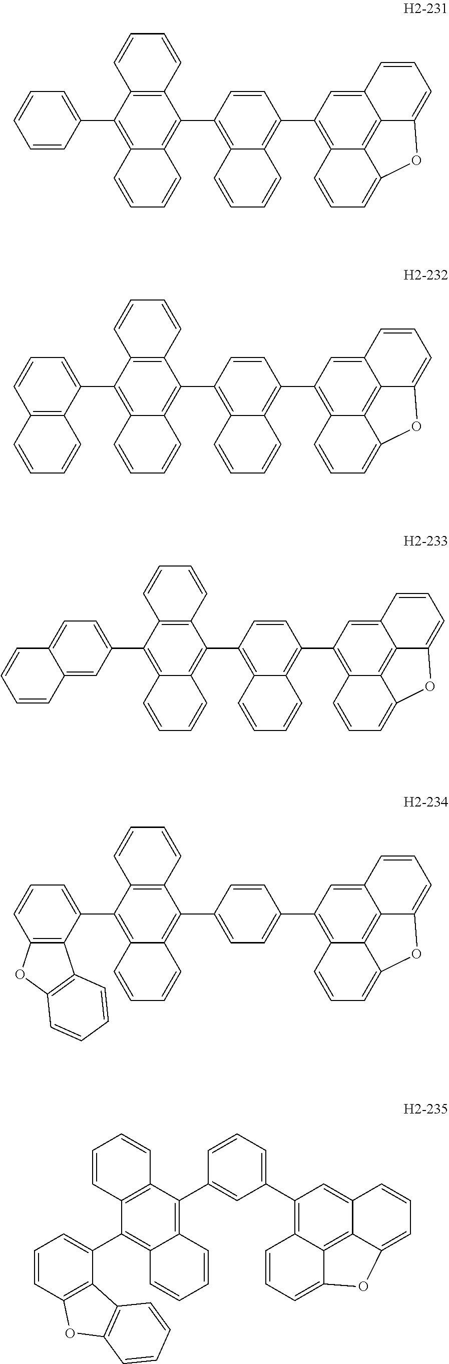 [SMILES: c1ccc(-c2c3ccccc3c(-c3ccc(-c4cc5cccc6oc7cccc4c7c56)c4ccccc34)c3ccccc23)cc1 ; c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1]

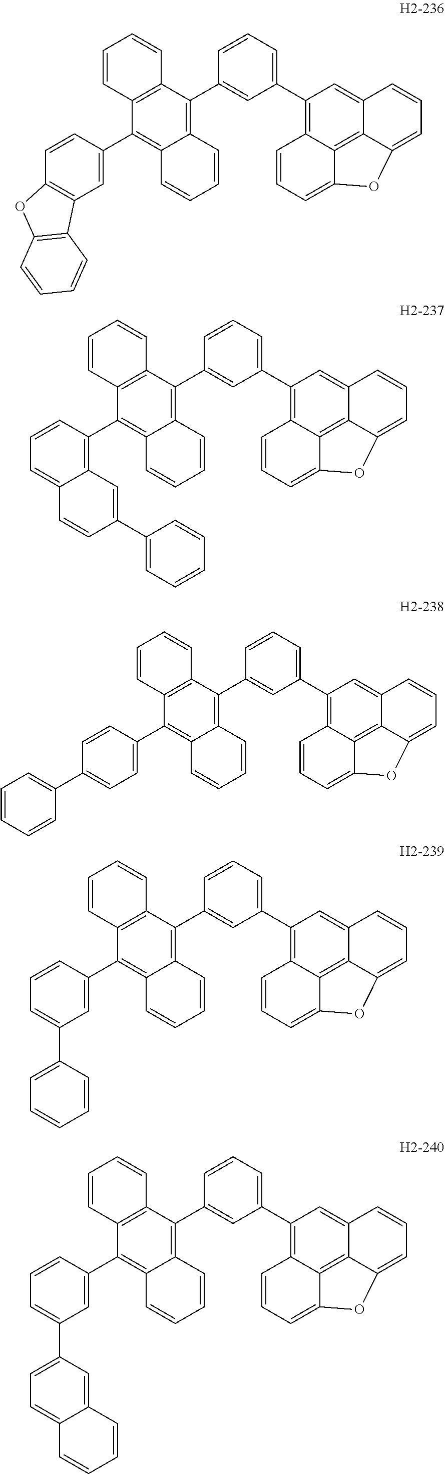

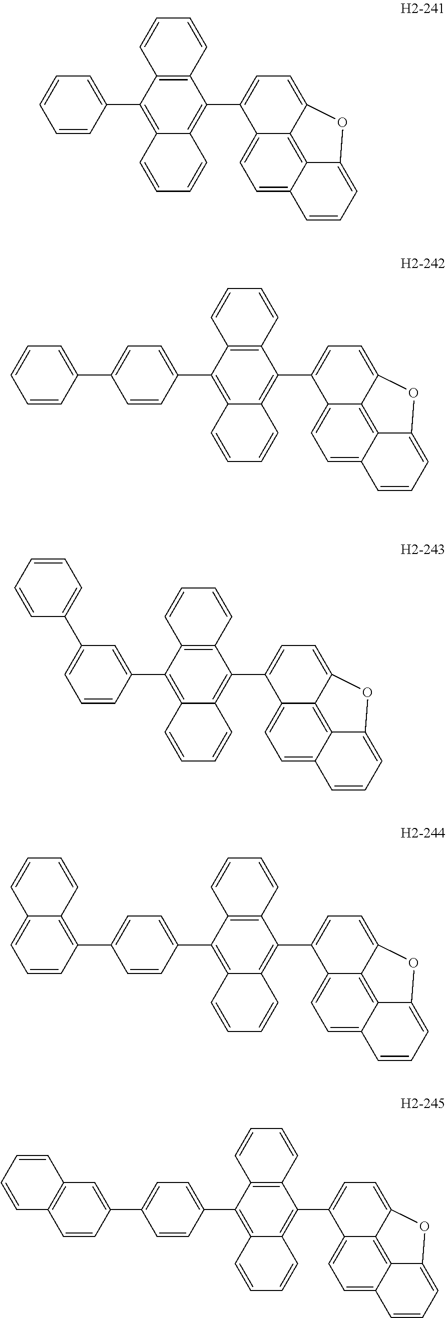 [SMILES: c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1 ; c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1]

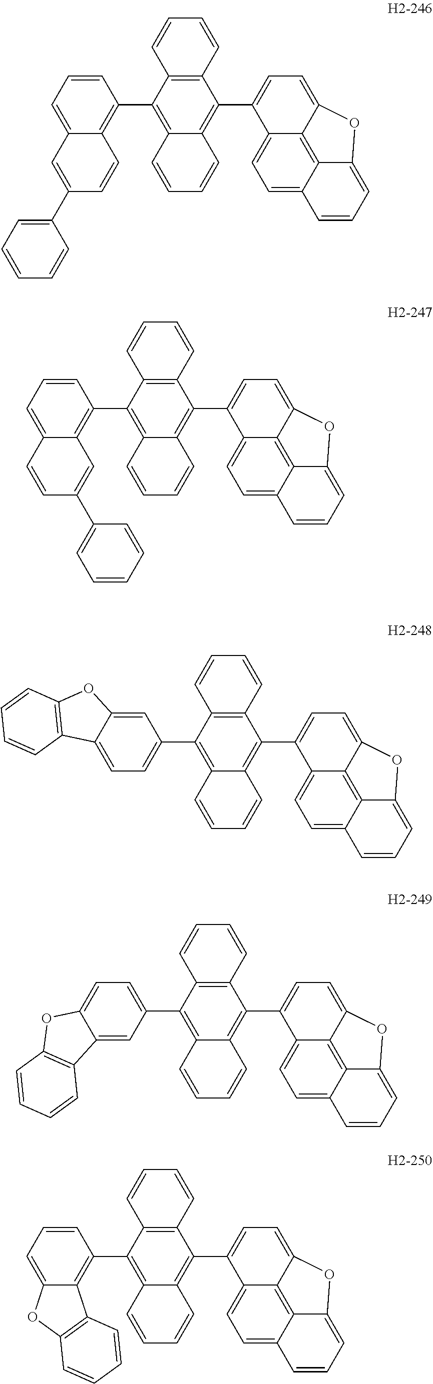 [SMILES: c1ccc(-c2ccc3c(-c4c5ccccc5c(-c5ccc6oc7cccc8ccc5c6c87)c5ccccc45)cccc3c2)cc1 ; c1ccc(-c2ccc3cccc(-c4c5ccccc5c(-c5ccc6oc7cccc8ccc5c6c87)c5ccccc45)c3c2)cc1 ; c1ccc2c(c1)oc1cc(-c3c4ccccc4c(-c4ccc5oc6cccc7ccc4c5c76)c4ccccc34)ccc12 ; c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12]

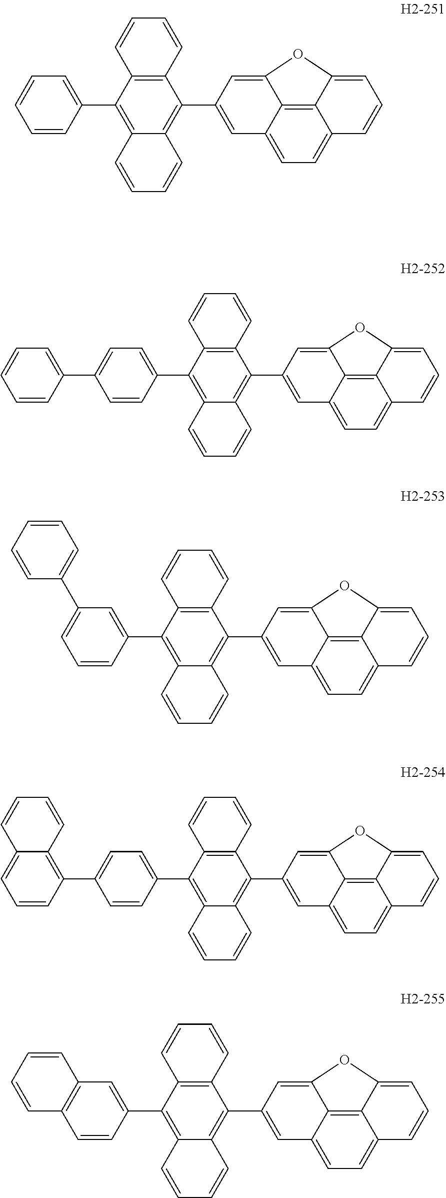 [SMILES: c1ccc(-c2c3ccccc3c(-c3cc4ccc5cccc6oc(c3)c4c56)c3ccccc23)cc1 ; c1ccc(-c2ccc(-c3c4ccccc4c(-c4cc5ccc6cccc7oc(c4)c5c67)c4ccccc34)cc2)cc1 ; c1ccc(-c2cccc(-c3c4ccccc4c(-c4cc5ccc6cccc7oc(c4)c5c67)c4ccccc34)c2)cc1 ; c1ccc2c(-c3ccc(-c4c5ccccc5c(-c5cc6ccc7cccc8oc(c5)c6c78)c5ccccc45)cc3)cccc2c1 ; c1ccc2cc(-c3c4ccccc4c(-c4cc5ccc6cccc7oc(c4)c5c67)c4ccccc34)ccc2c1]

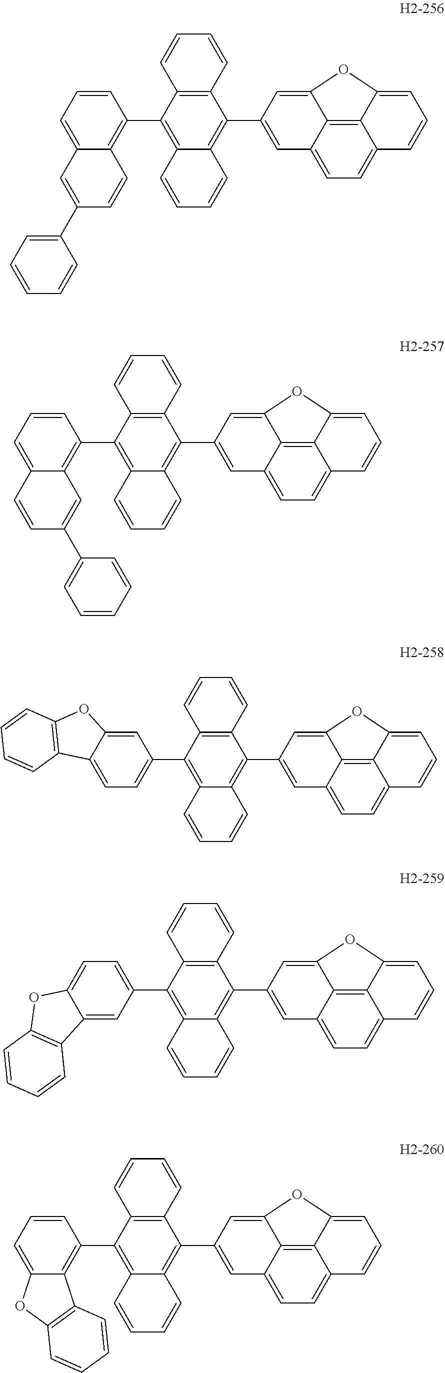 [SMILES: c1ccc(-c2ccc3c(-c4c5ccccc5c(-c5cc6ccc7cccc8oc(c5)c6c78)c5ccccc45)cccc3c2)cc1 ; c1ccc(-c2ccc3cccc(-c4c5ccccc5c(-c5cc6ccc7cccc8oc(c5)c6c78)c5ccccc45)c3c2)cc1 ; c1ccc2c(c1)oc1cc(-c3c4ccccc4c(-c4cc5ccc6cccc7oc(c4)c5c67)c4ccccc34)ccc12 ; c1ccc2c(c1)oc1ccc(-c3c4ccccc4c(-c4cc5ccc6cccc7oc(c4)c5c67)c4ccccc34)cc12 ; c1ccc2c(c1)oc1cccc(-c3c4ccccc4c(-c4cc5ccc6cccc7oc(c4)c5c67)c4ccccc34)c12]

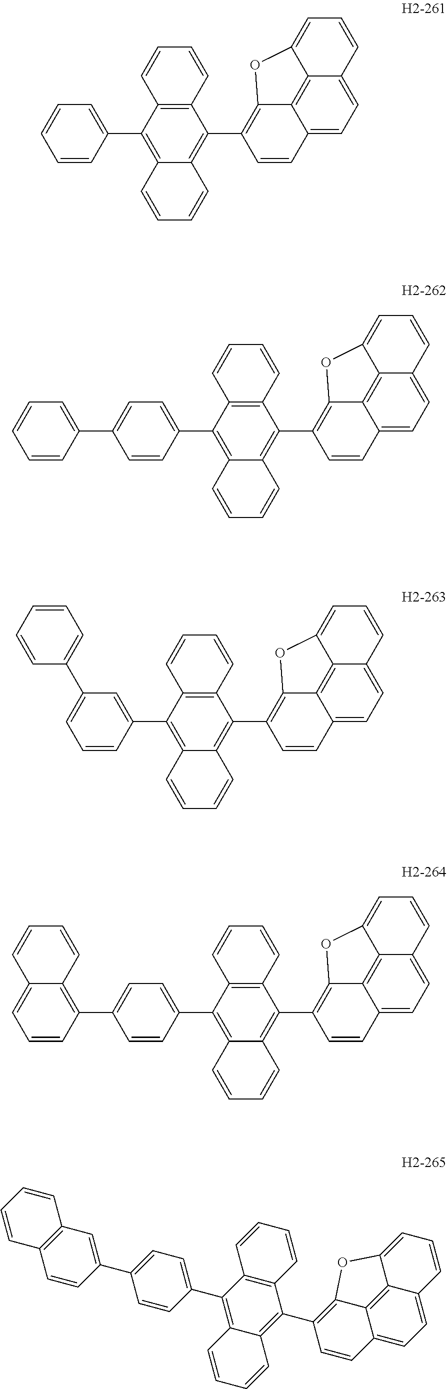 [SMILES: c1ccc(-c2c3ccccc3c(-c3ccc4ccc5cccc6oc3c4c56)c3ccccc23)cc1 ; c1ccc(-c2ccc(-c3c4ccccc4c(-c4ccc5ccc6cccc7oc4c5c67)c4ccccc34)cc2)cc1 ; c1ccc(-c2cccc(-c3c4ccccc4c(-c4ccc5ccc6cccc7oc4c5c67)c4ccccc34)c2)cc1 ; c1ccc2c(-c3ccc(-c4c5ccccc5c(-c5ccc6ccc7cccc8oc5c6c78)c5ccccc45)cc3)cccc2c1 ; c1ccc2cc(-c3ccc(-c4c5ccccc5c(-c5ccc6ccc7cccc8oc5c6c78)c5ccccc45)cc3)ccc2c1]

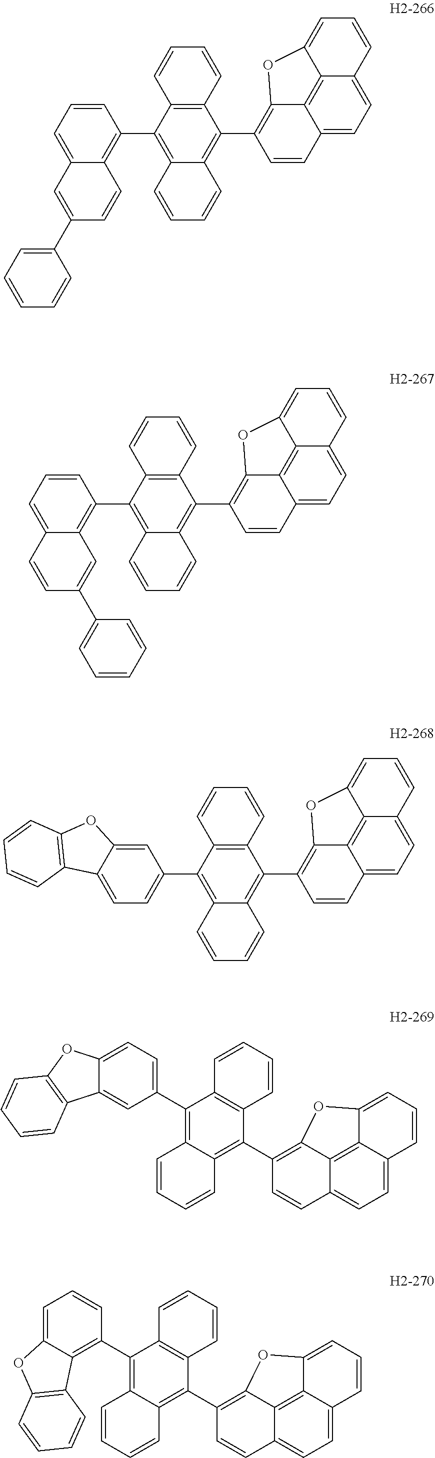 [SMILES: c1ccc(-c2ccc3c(-c4c5ccccc5c(-c5ccc6ccc7cccc8oc5c6c78)c5ccccc45)cccc3c2)cc1 ; c1ccc(-c2ccc3cccc(-c4c5ccccc5c(-c5ccc6ccc7cccc8oc5c6c78)c5ccccc45)c3c2)cc1 ; c1ccc2c(c1)oc1cc(-c3c4ccccc4c(-c4ccc5ccc6cccc7oc4c5c67)c4ccccc34)ccc12 ; c1ccc2c(c1)oc1ccc(-c3c4ccccc4c(-c4ccc5ccc6cccc7oc4c5c67)c4ccccc34)cc12 ; c1ccc2c(c1)oc1cccc(-c3c4ccccc4c(-c4ccc5ccc6cccc7oc4c5c67)c4ccccc34)c12]

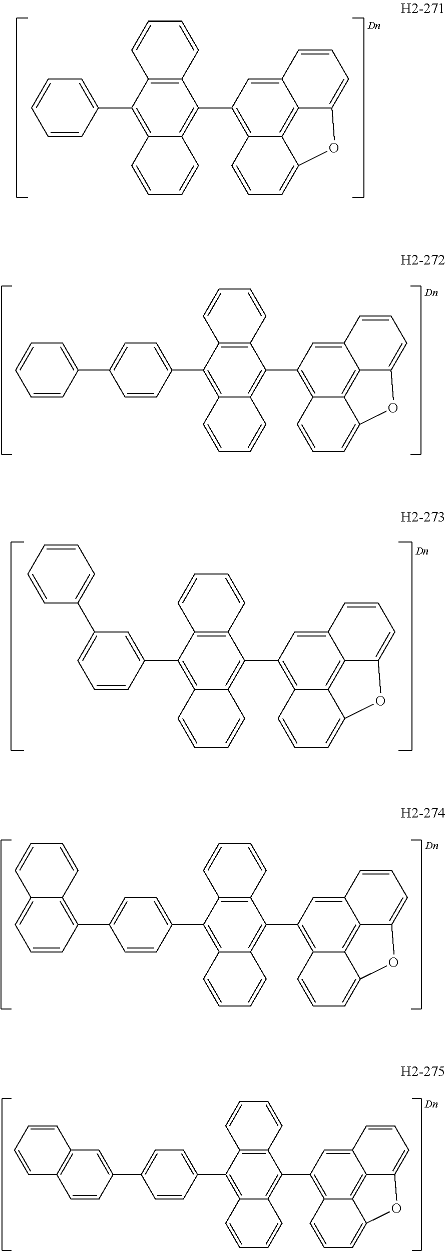

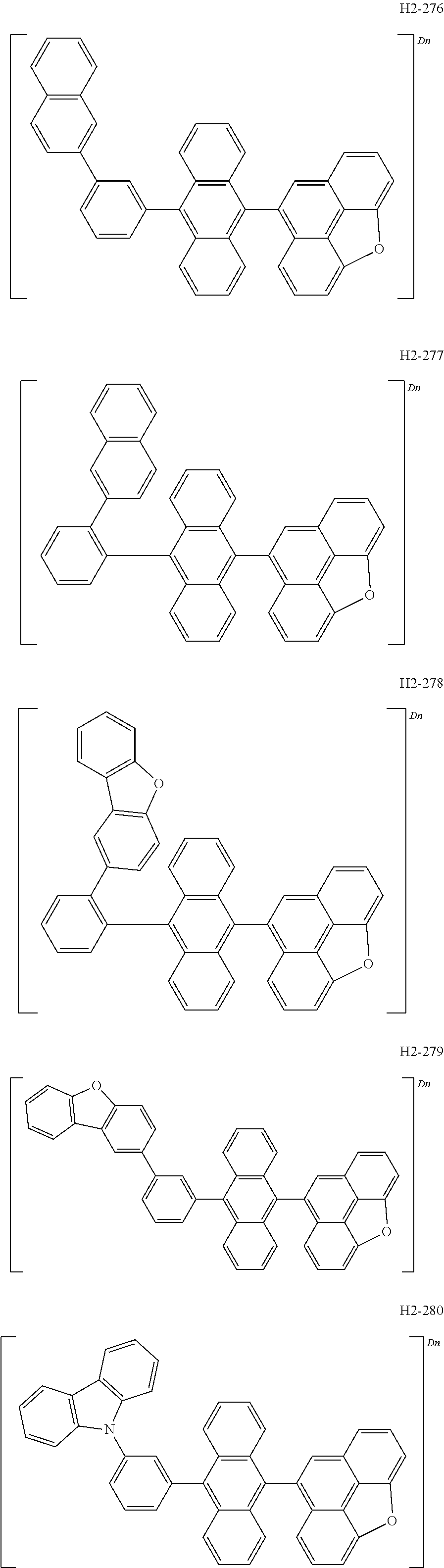

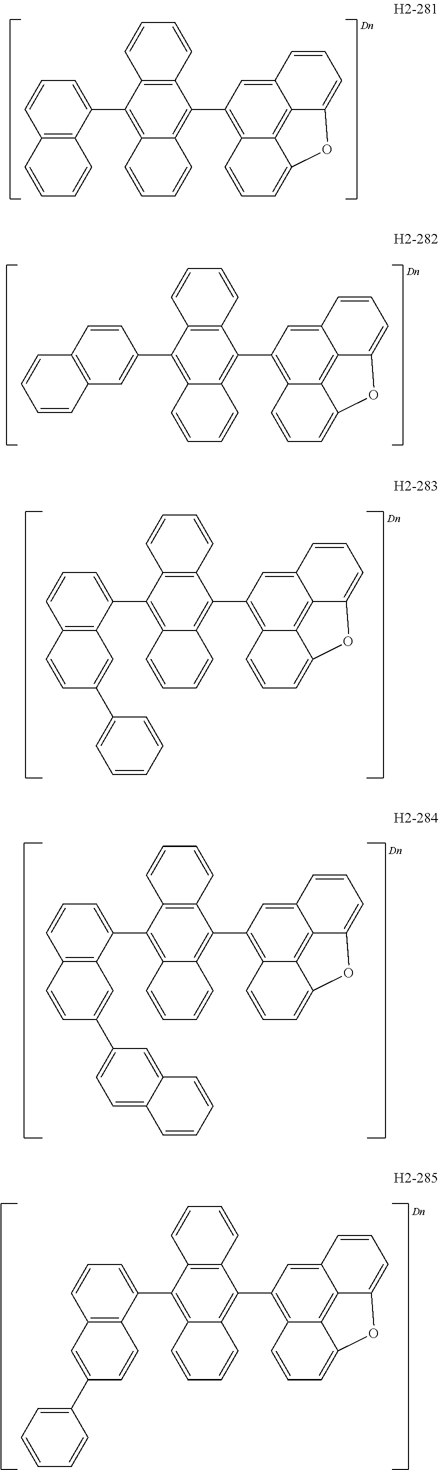

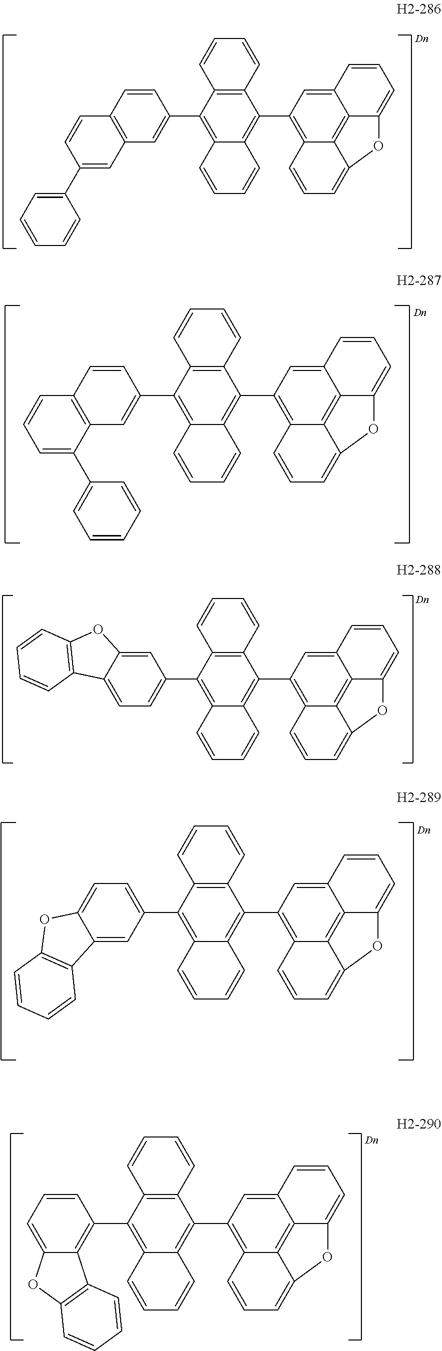

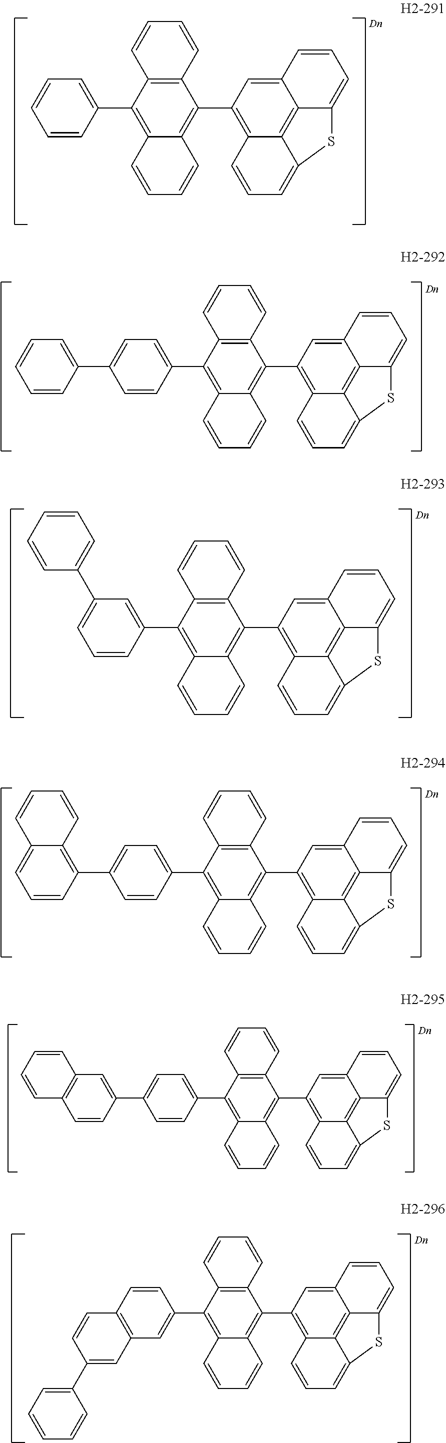

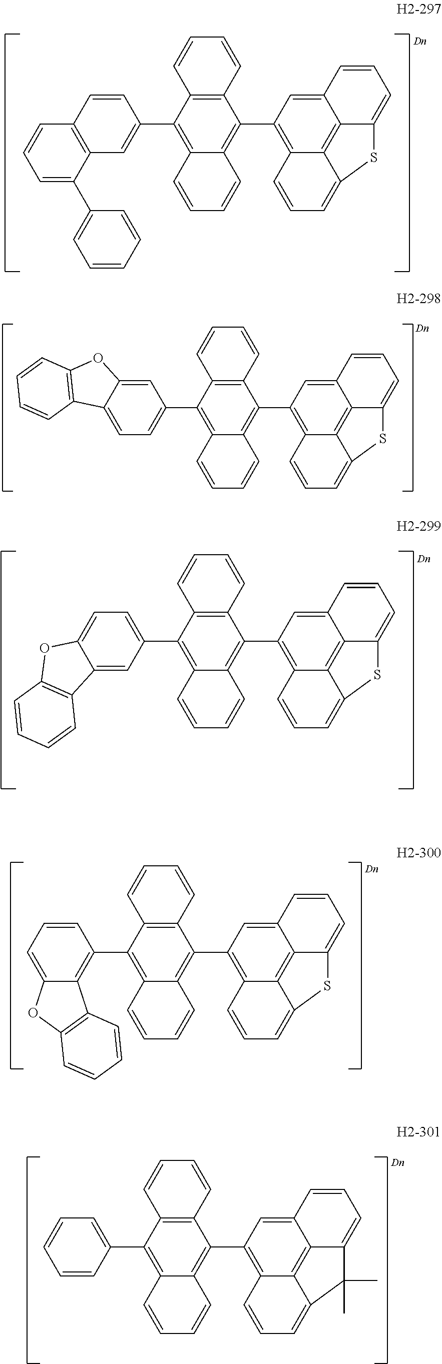

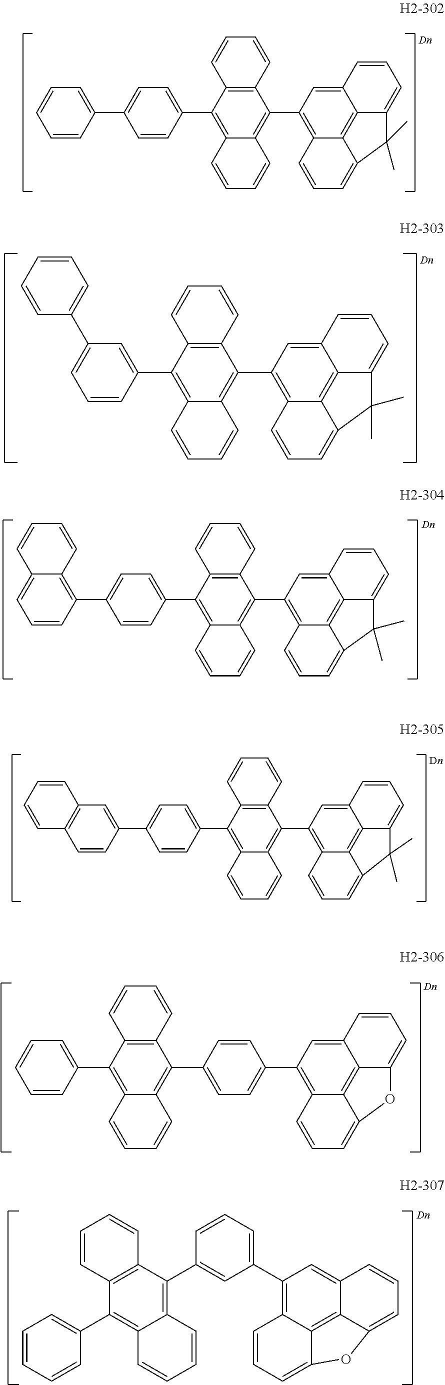

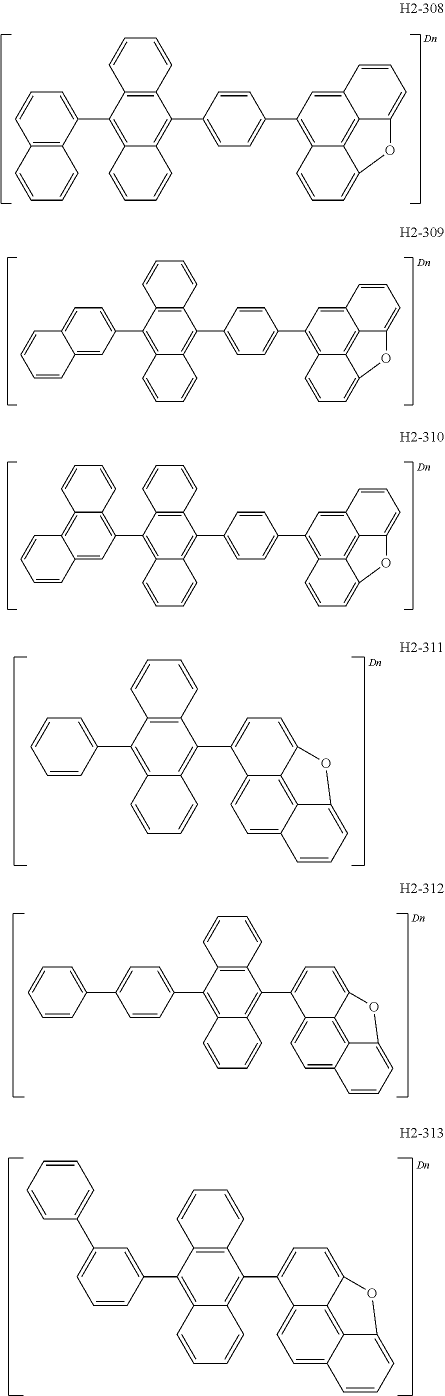

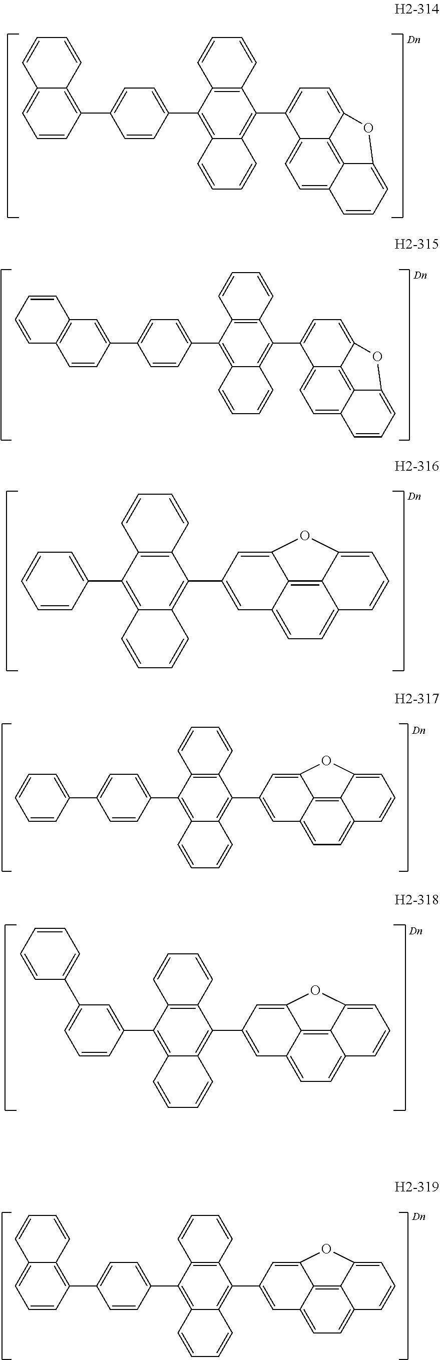

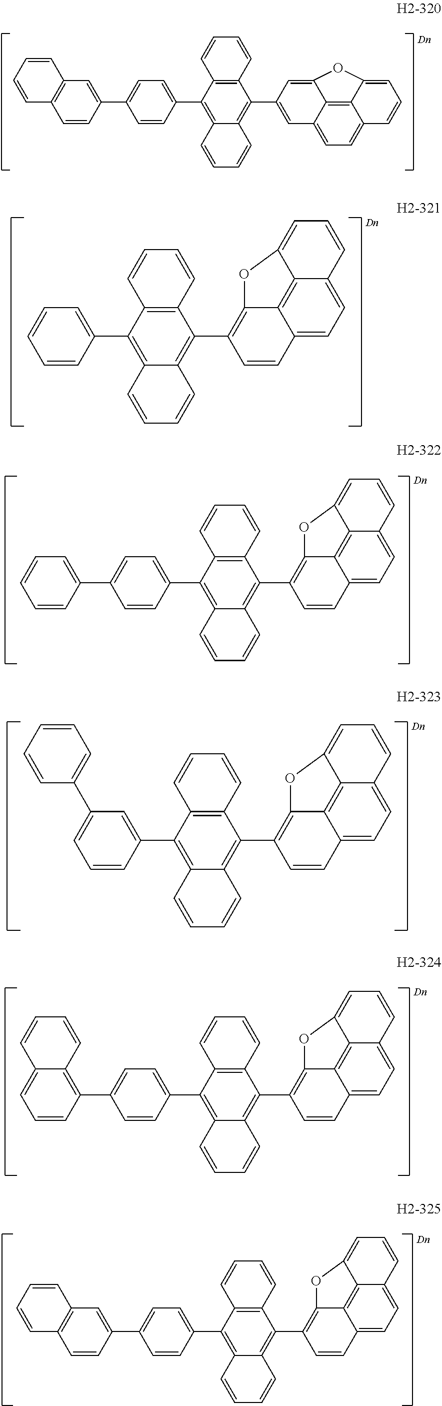

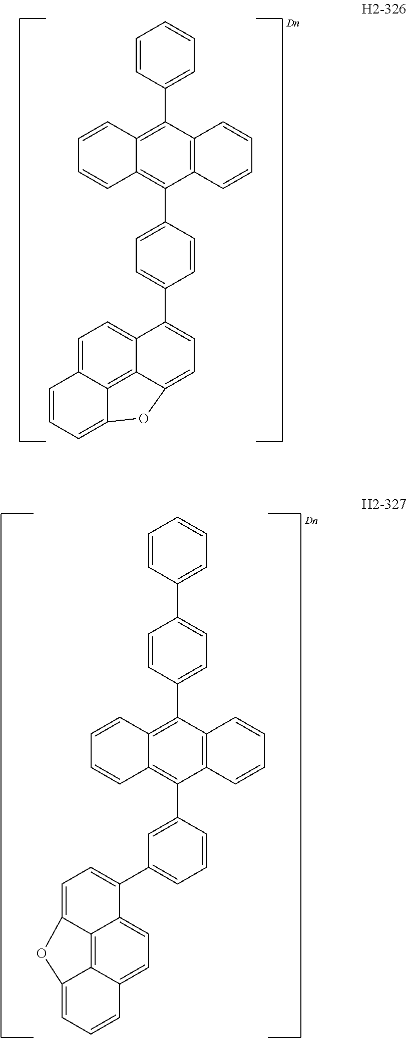

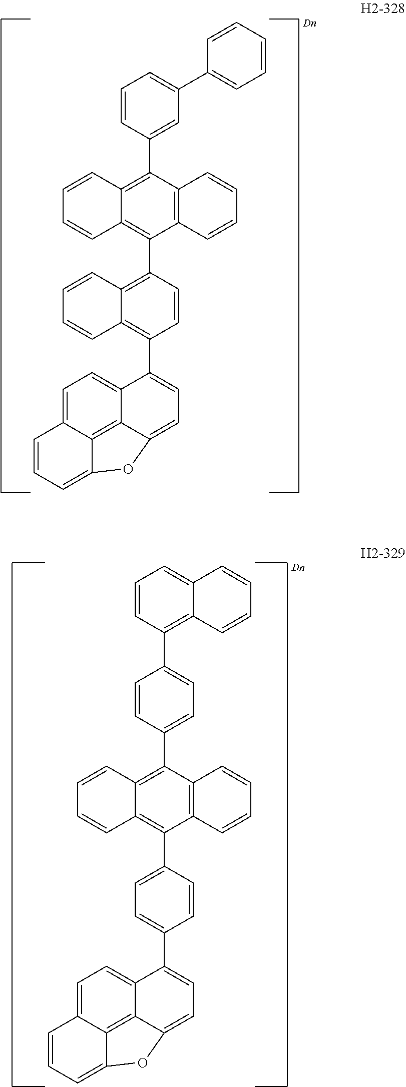

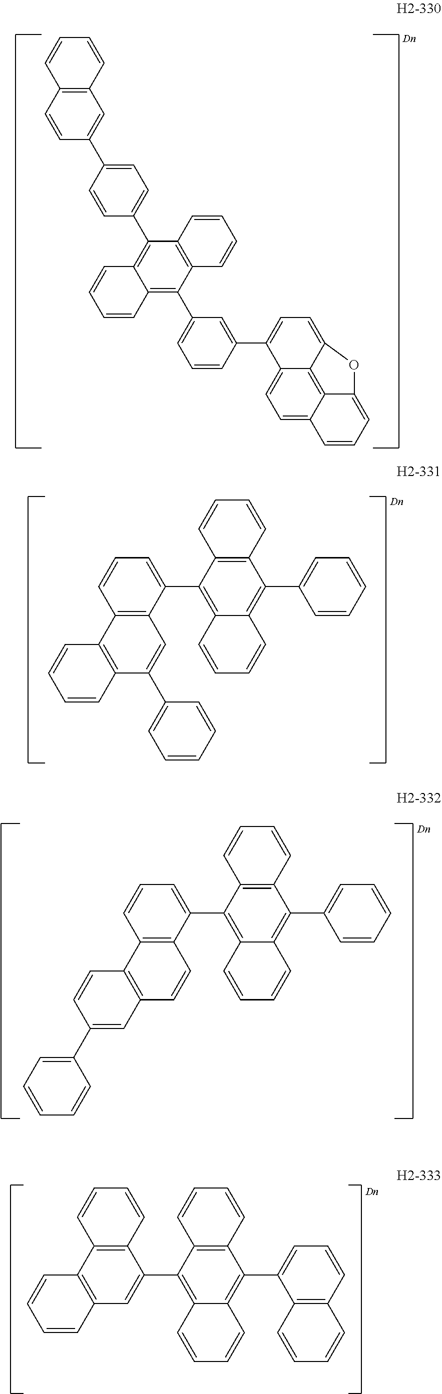

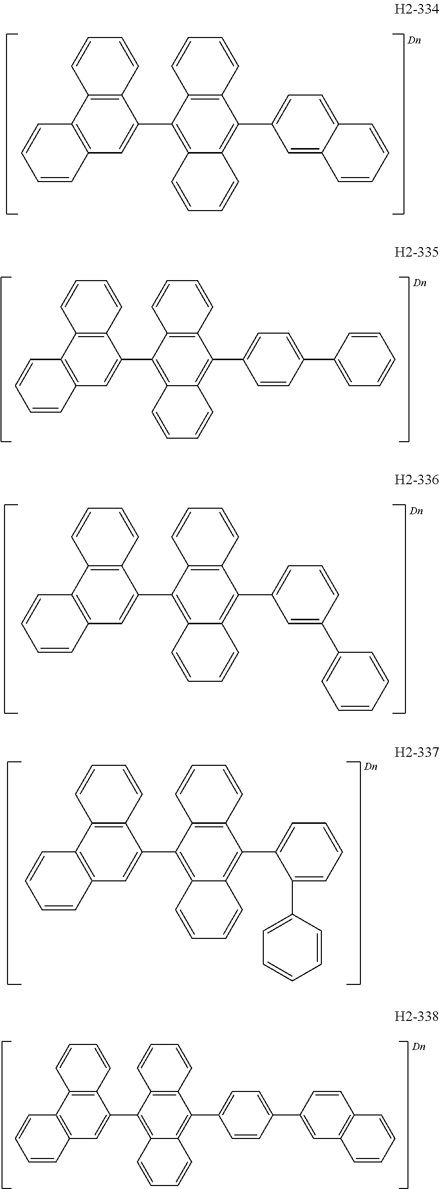

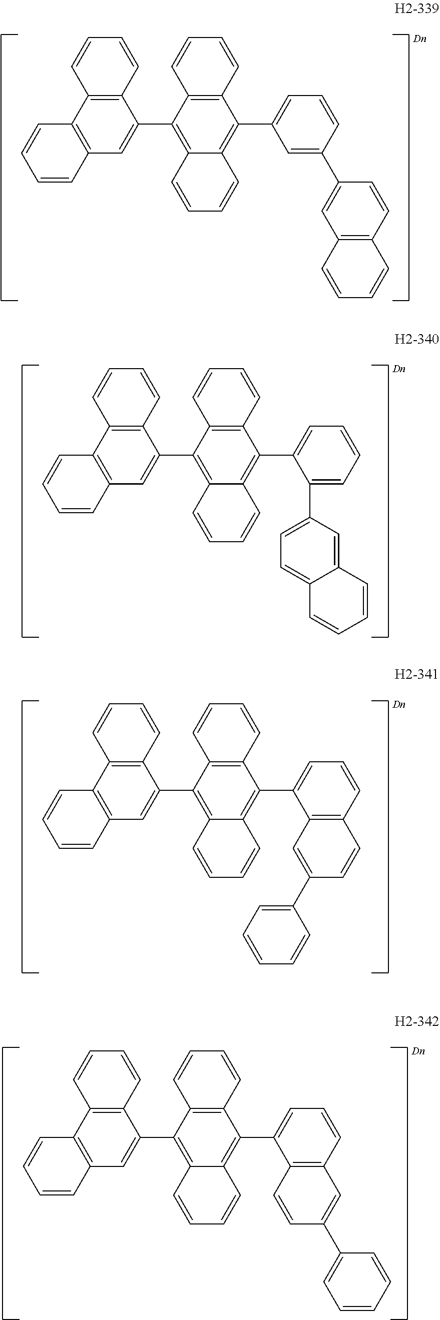

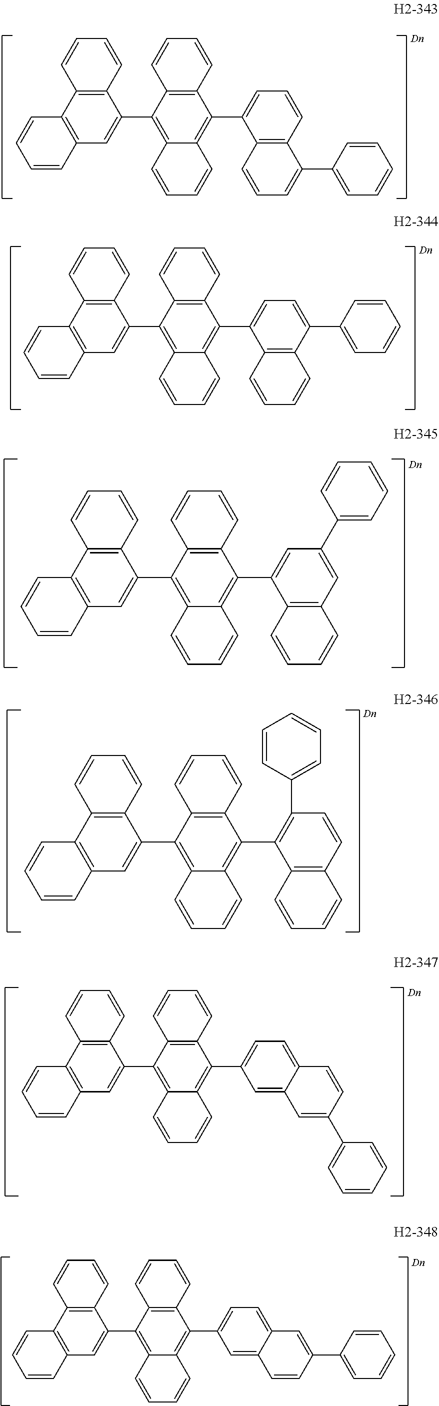

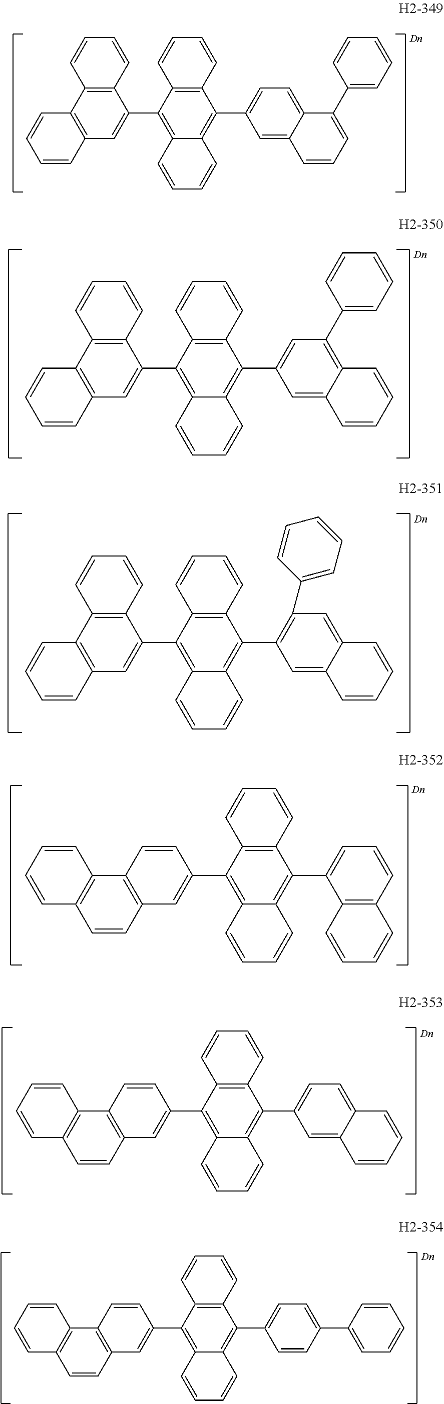

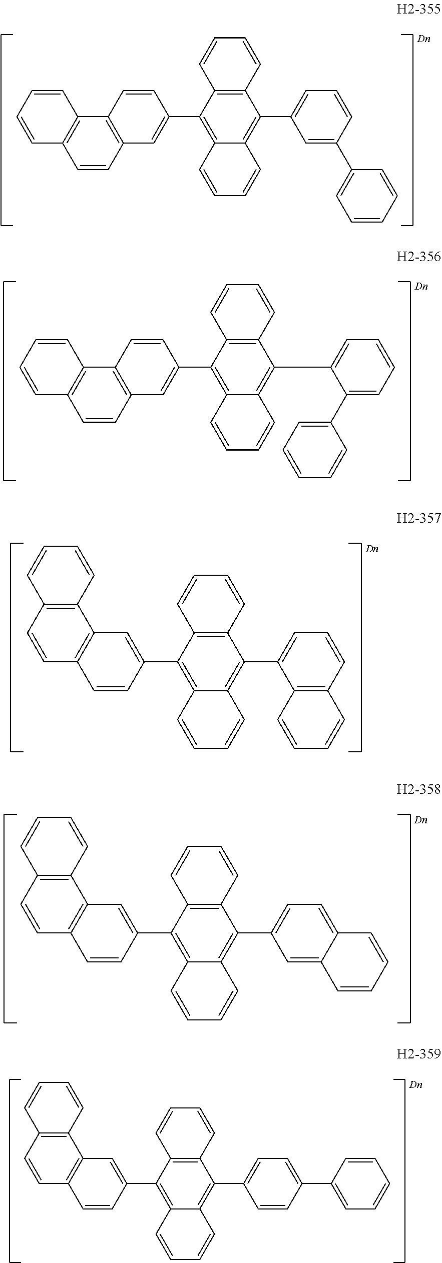

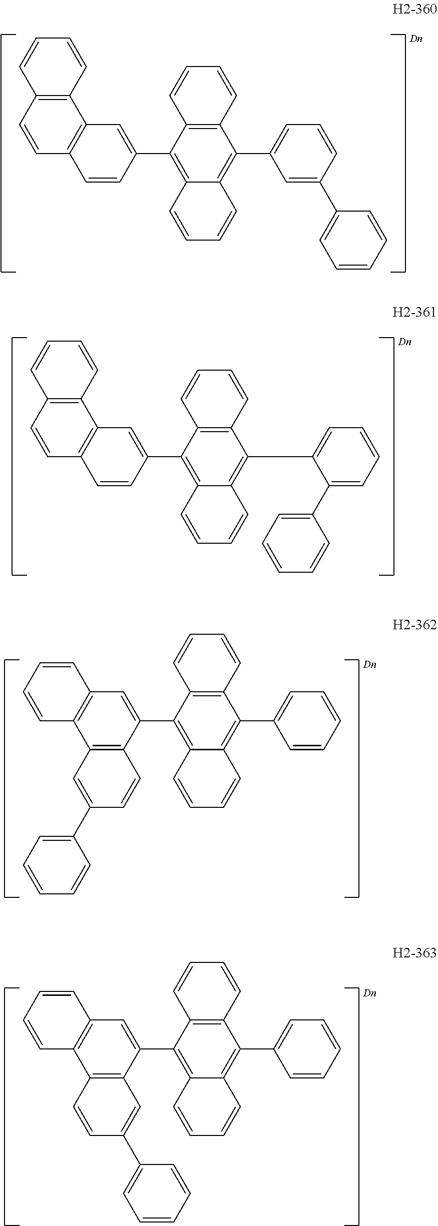

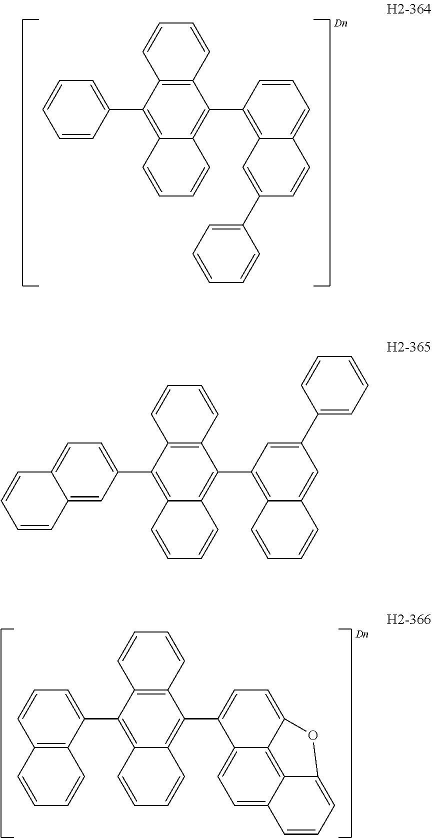

wherein “Dₙ” in the above compounds means that n number of hydrogens is replaced by deuterium, wherein n represents an integer of 1 or more and the upper limit of n is determined by the number of hydrogens that can be substituted in each compound.

The compound represented by Formula 1 or 2 according to the present disclosure can be manufactured by referring to a synthesis method known to those skilled in the art, for example, the synthesis method disclosed in Korean Patent Application Laid-open Nos. 2021-0046437 and 2010-0109060, Chinese Patent Application Laid-open No. 110294663, etc., but is not limited thereto.

In addition, the deuterated compound of Formulas 1 and 2 can be prepared using a deuterated precursor material in a similar manner, or more generally can be prepared by treating a non-deuterated compound with a deuterated solvent, D6-benzene in the presence of a Lewis acid H/D exchange catalyst such as aluminium trichloride or ethyl aluminium chloride. In addition, the degree of deuteration can be controlled by varying reaction conditions such as reaction temperature. For example, the number of deuterium in Formulas 1 and 2 can be adjusted by controlling the reaction temperature and time, the equivalent of acid, etc.

According to another embodiment of the present disclosure, the present disclosure provides an organic electroluminescent compound represented by the following Formula 11.

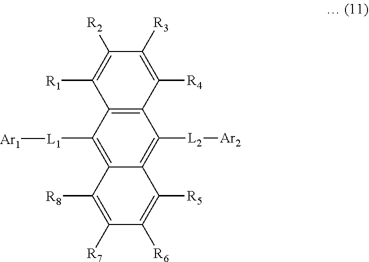

In Formula 11,
R₁ to R₈ each independently represent hydrogen or deuterium; provided that at least one of R₁ to R₈ is deuterium,
L₁ and L₂ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl, or a substituted or unsubstituted (3- to 30-membered)heteroaryl; and
Ar₂ is represented by the following Formula 1-A;

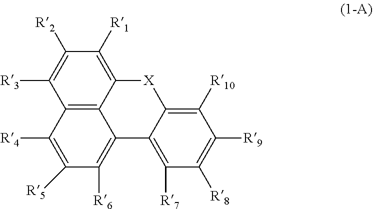

In Formula 1-A
X represents —NR″—, —Se—, —O—, or —S—;
R′₁ to R′₁₀ each independently represent hydrogen, deuterium, a substituted or unsubstituted (C₆-C₃₀)aryl, or a substituted or unsubstituted (3- to 30-membered)heteroaryl; or may be linked to the adjacent substituents to form a substituted or unsubstituted (3- to 30-membered) mono- or polycyclic, alicyclic, aromatic ring, or a combination thereof; provided that at least one of R′₁ to R′₁₀ is deuterium; and
any one of R′₁ to R′₁₀ is linked to L₂;
provided that the cases where all hydrogens in the compound are replaced with deuterium are excluded.

In one embodiment, at least one of R₁, R₄, R₅, and R₃ in Formula 11 may be hydrogen.

In one embodiment, a residual rate of hydrogen in Formula 11 may be 20% to 60%, 30% to 60%, or 40% to 60% of the total amount of hydrogen. The compound with the residual rate of hydrogen is in the above ranges, the bond dissociation energy increases due to deuteration, thereby increasing the stability of the compound. Additionally, the organic electroluminescent compound according to the present can improve lifespan characteristics when used in an organic electroluminescent device with only minimal deuterium substitution.

In one embodiment, a residual rate of hydrogen in Formula 11 may be 30% to 50% of the total number of hydrogen.

In one embodiment, a residual rate of hydrogen in Formula 11 may be 20% to 30% of the total number of hydrogen.

According to one embodiment, the compound represented by Formula 11 may be more specifically illustrated by the following compounds, but is not limited thereto.

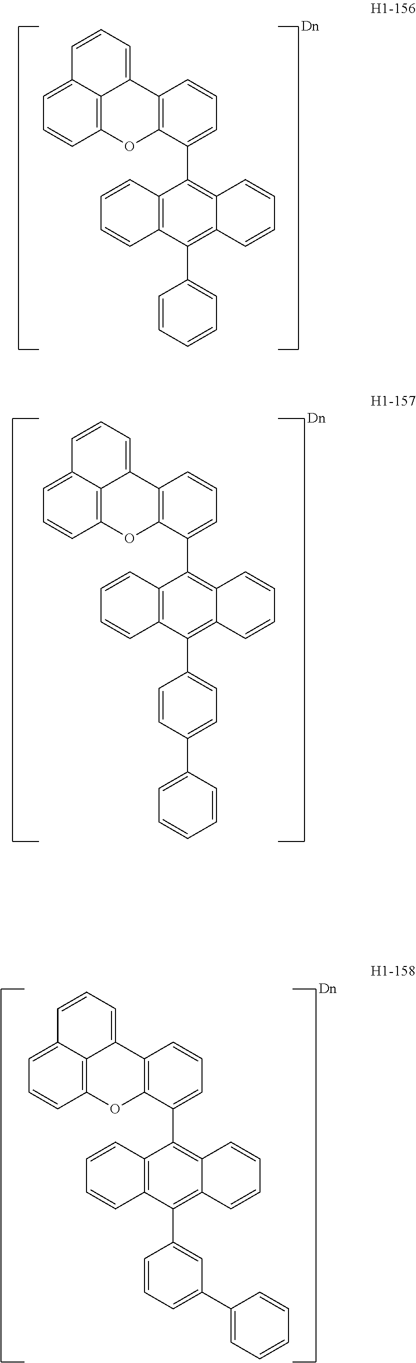

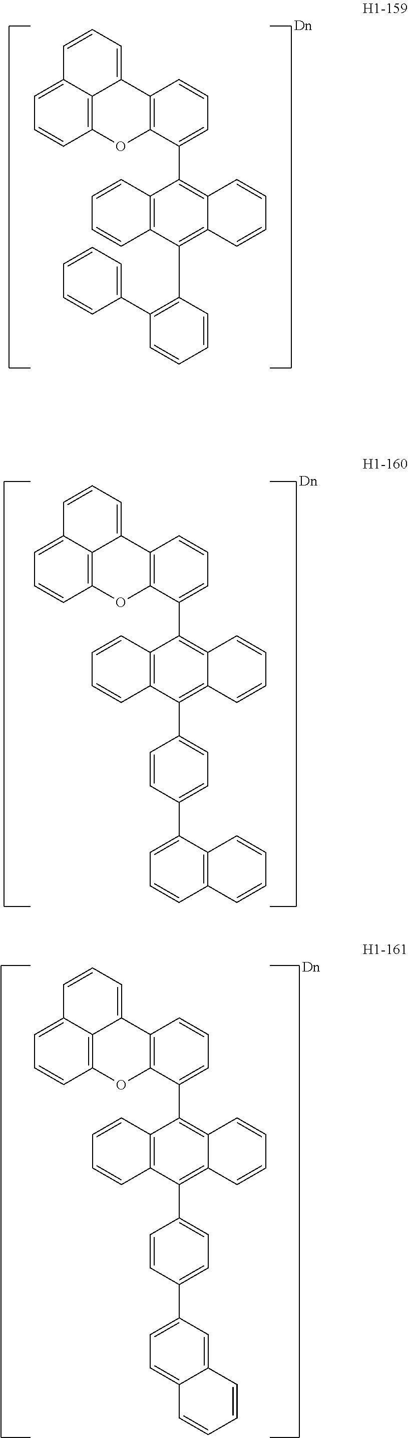

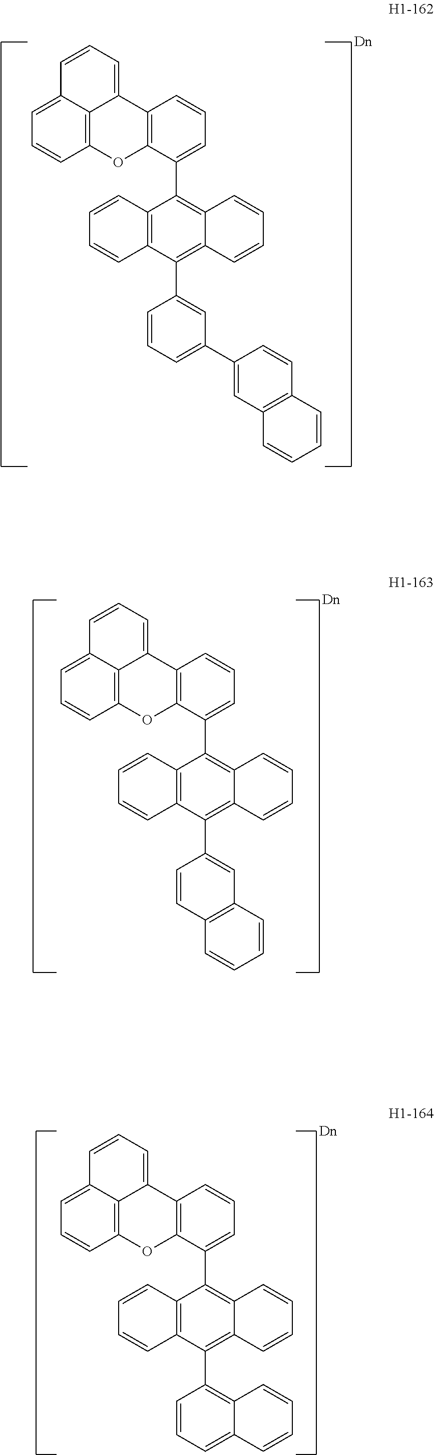

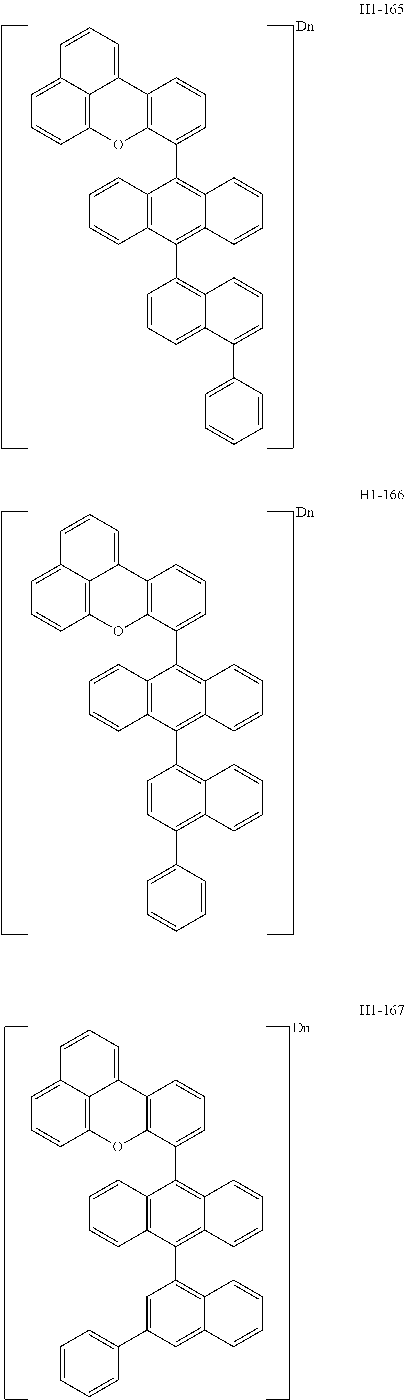

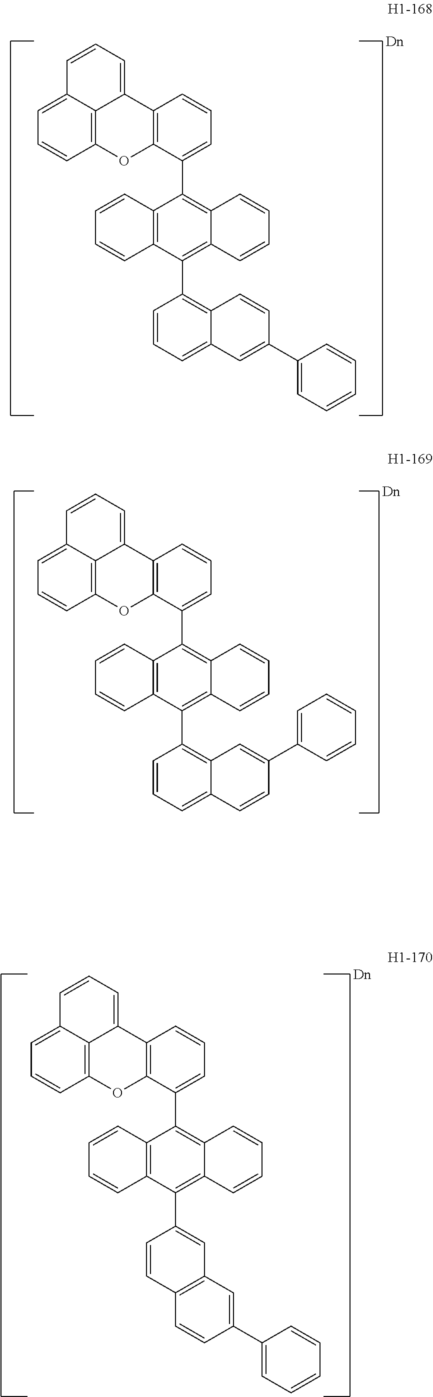

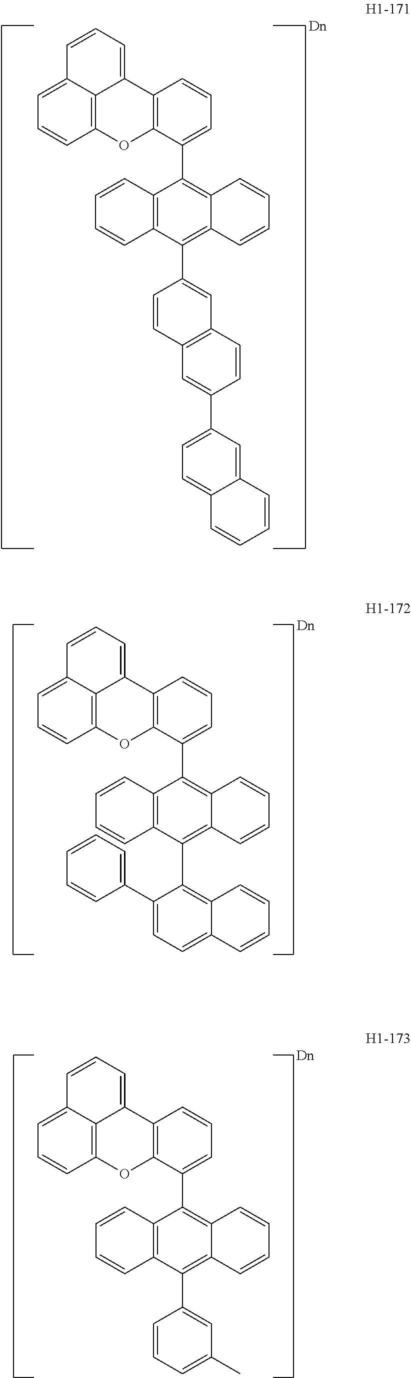

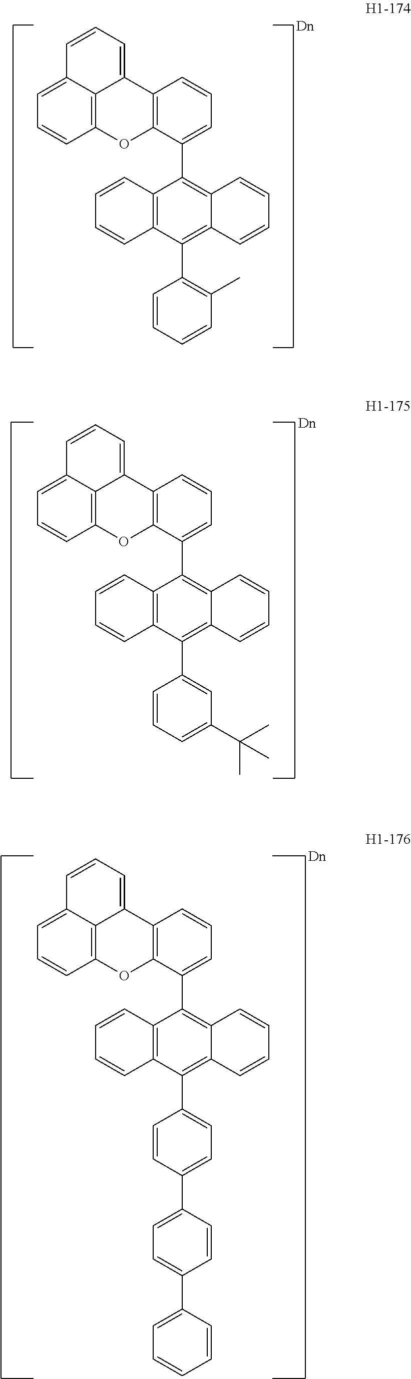

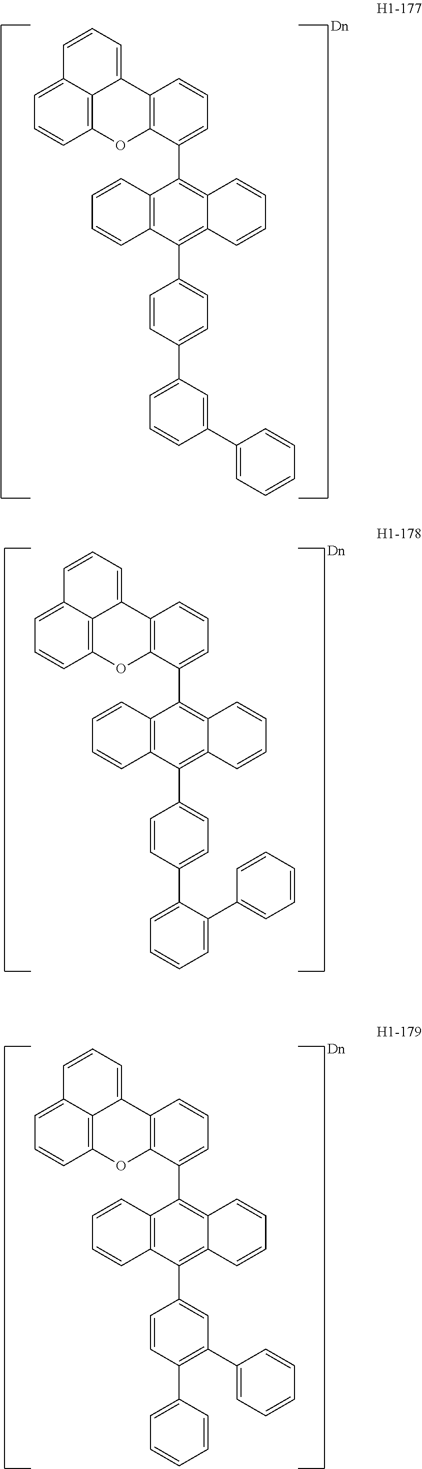

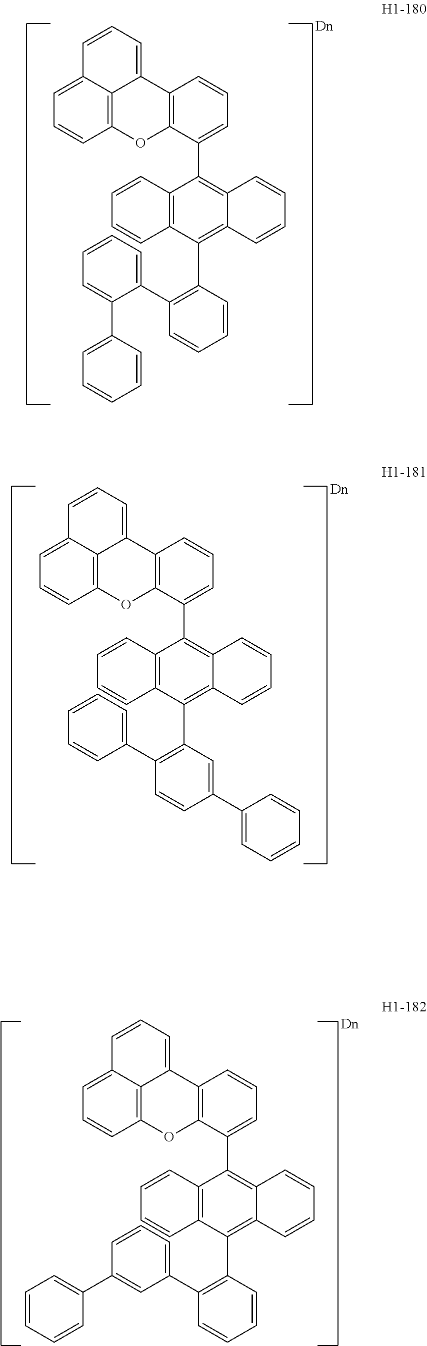

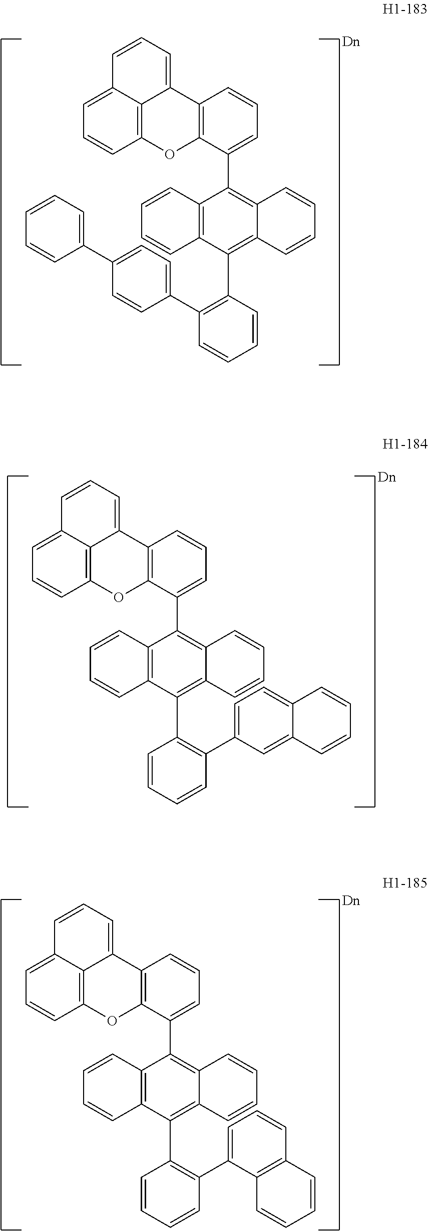

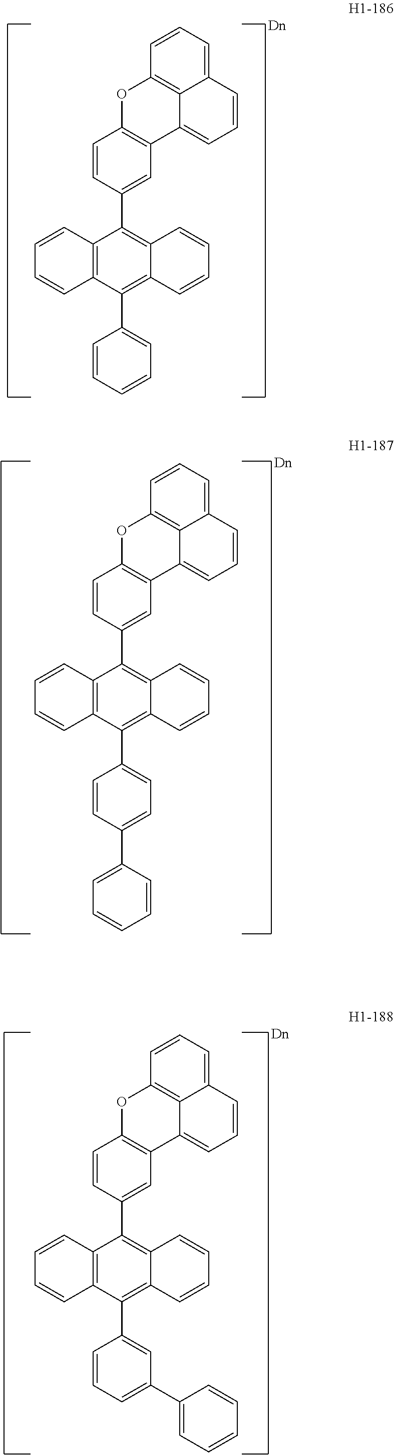

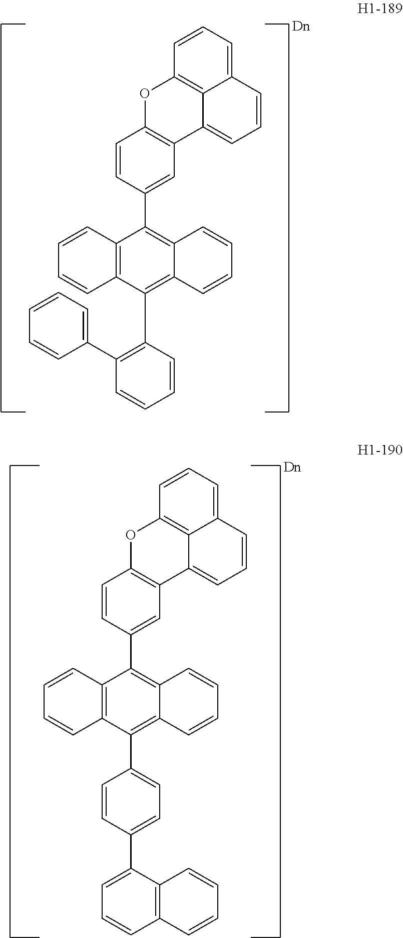

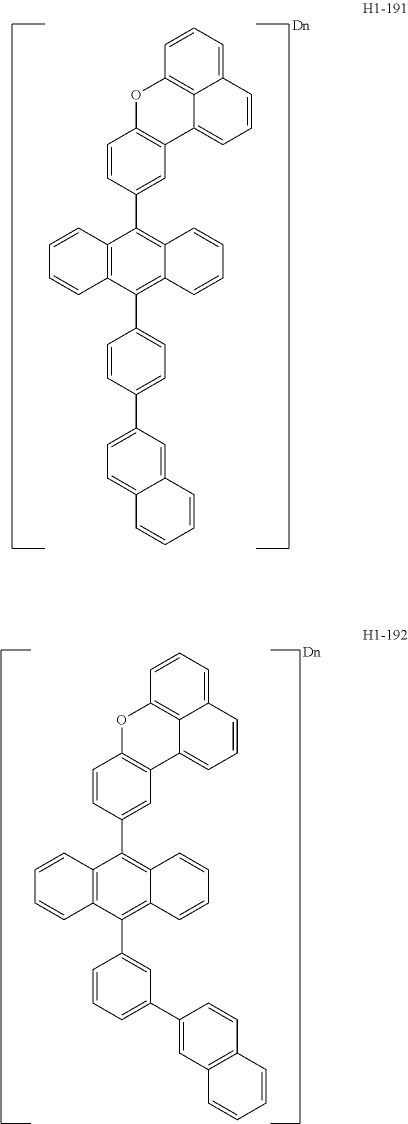

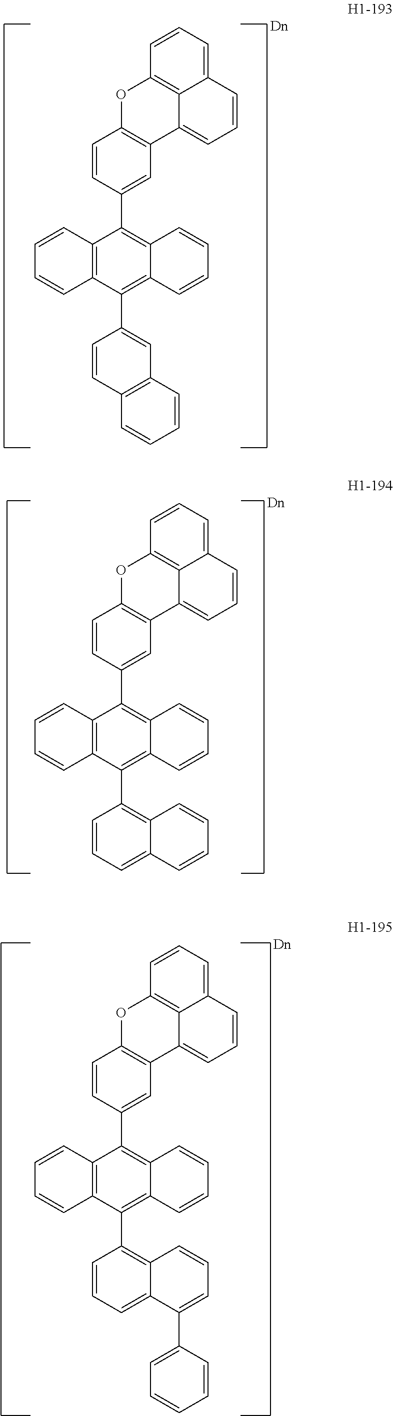

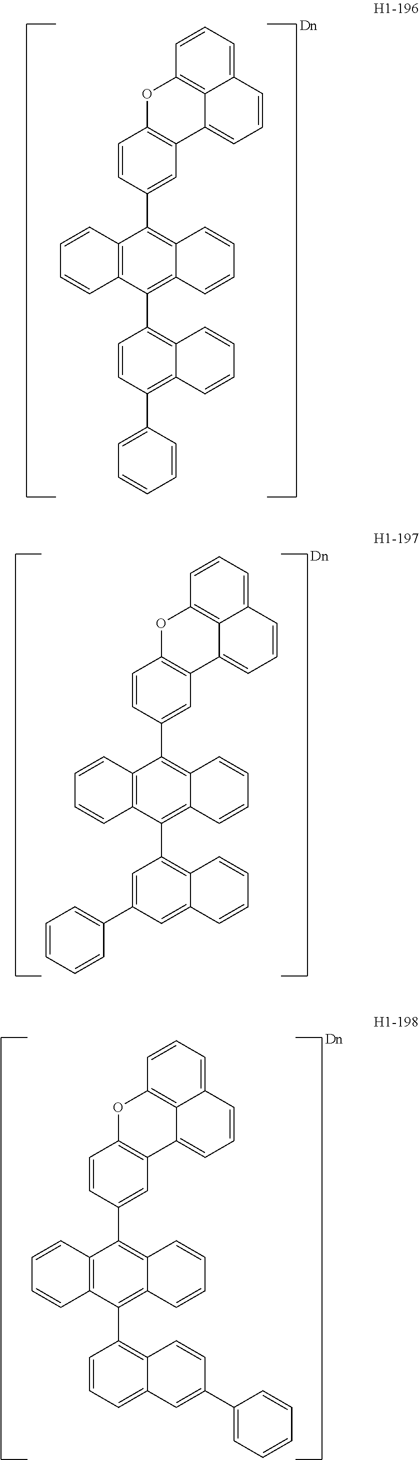

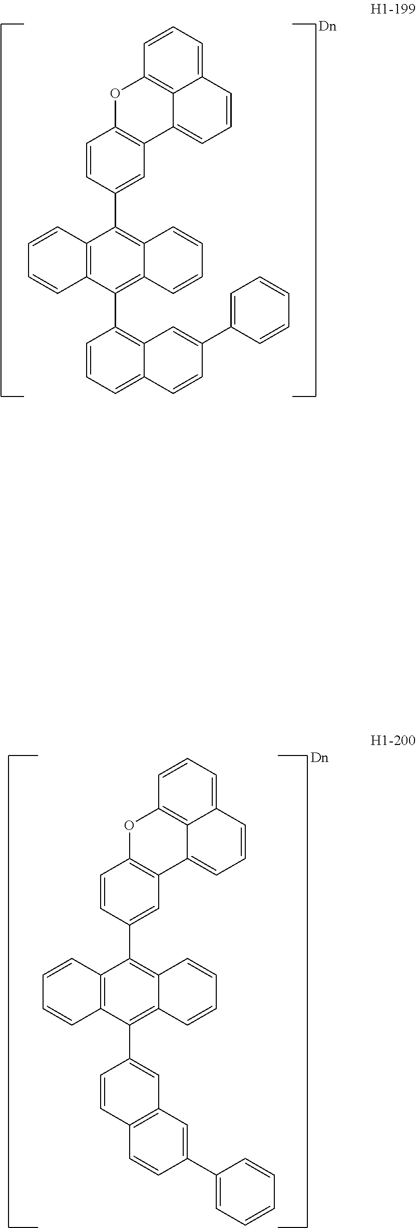

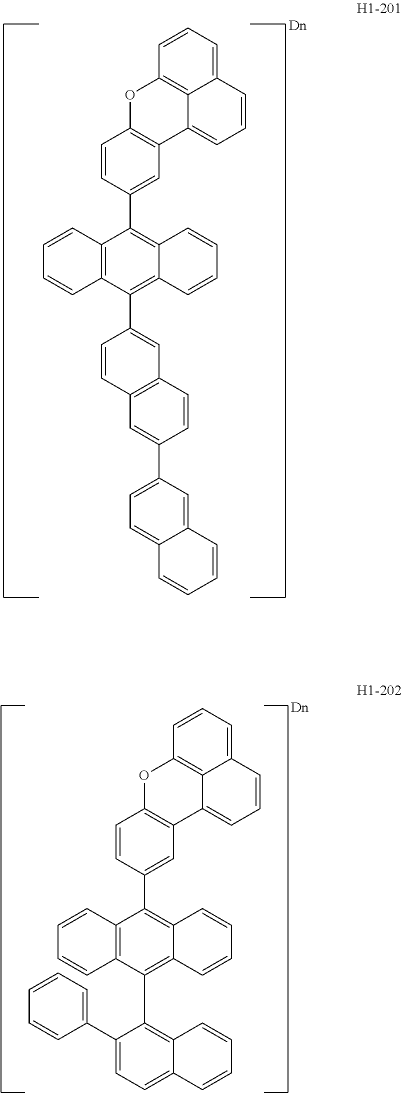

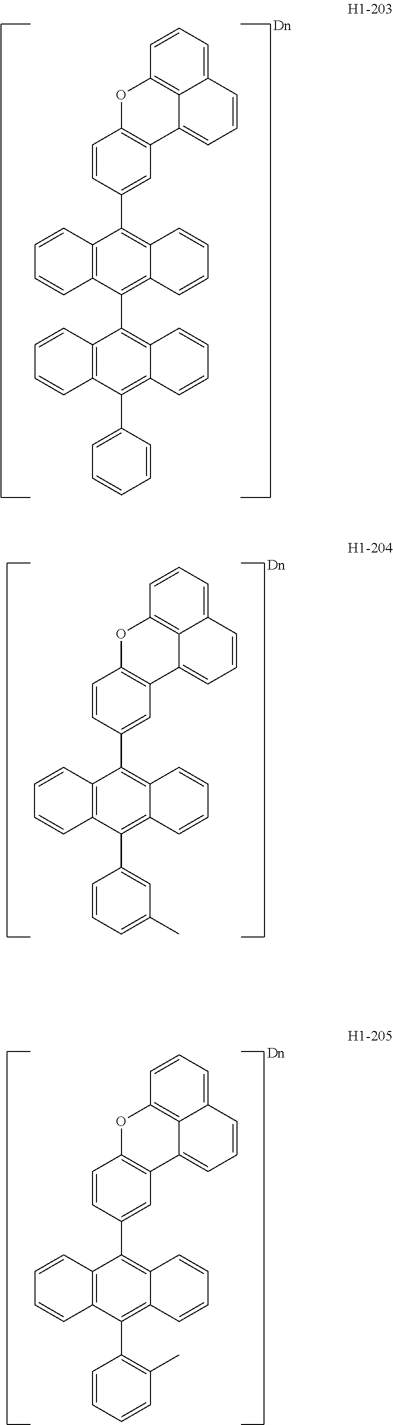

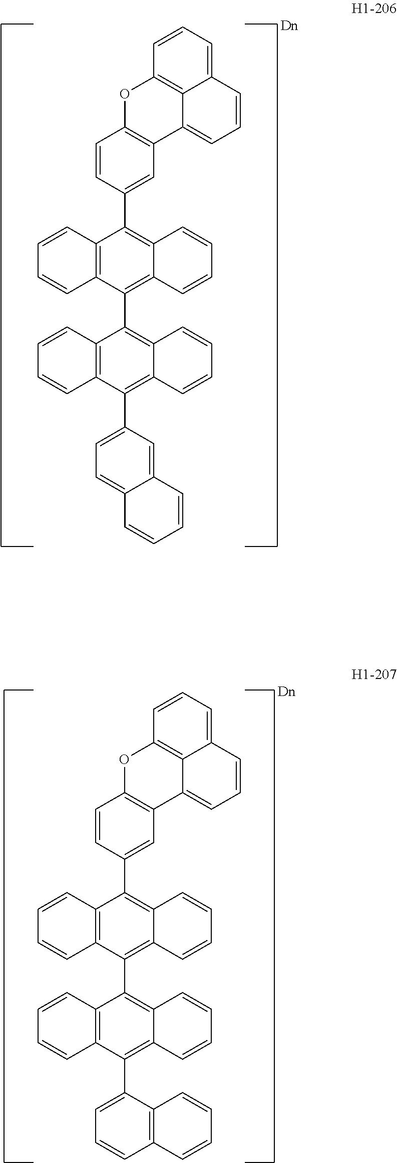

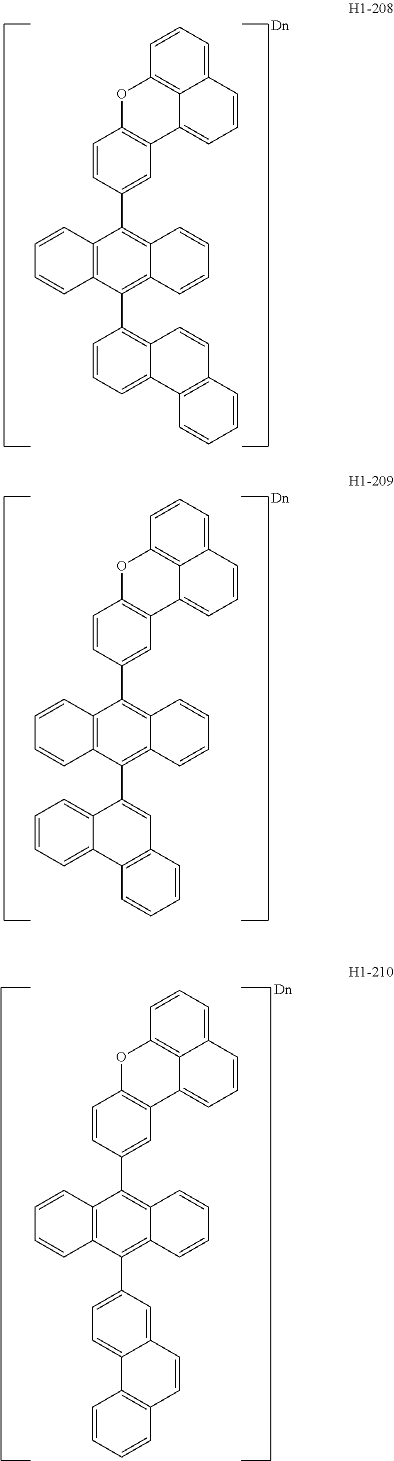

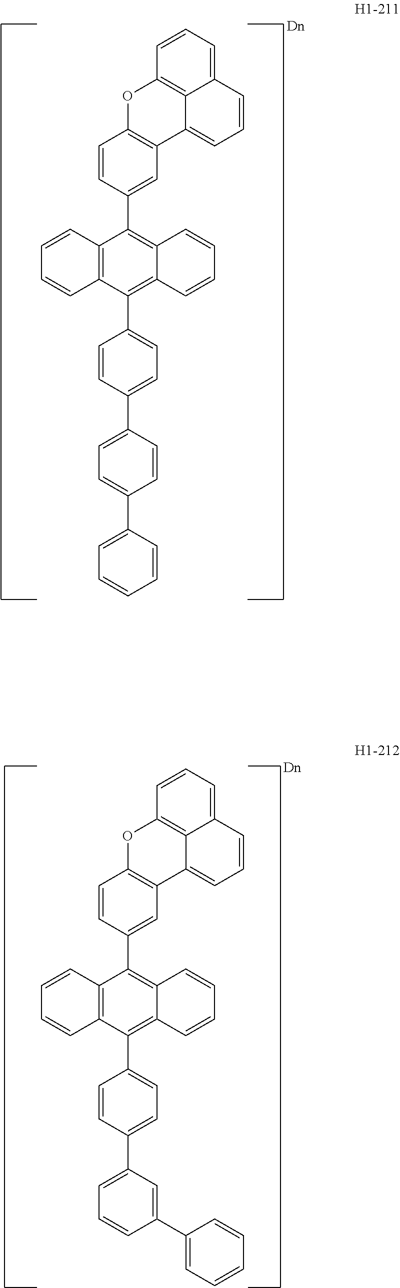

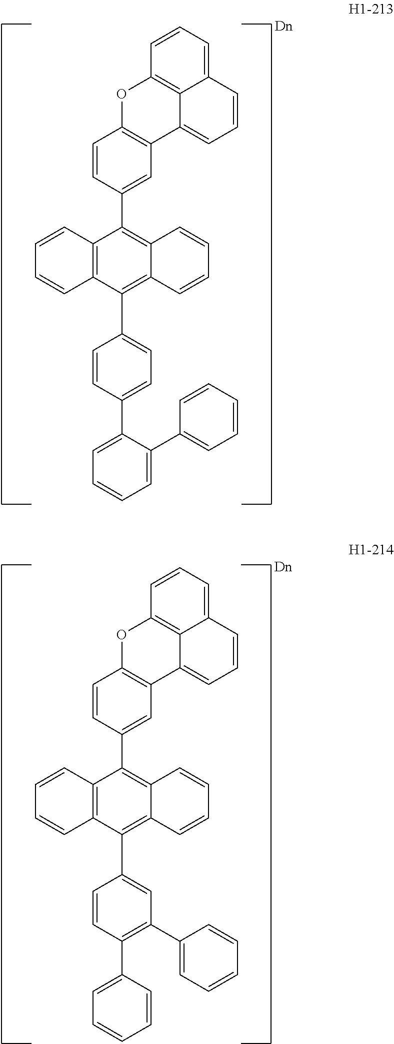

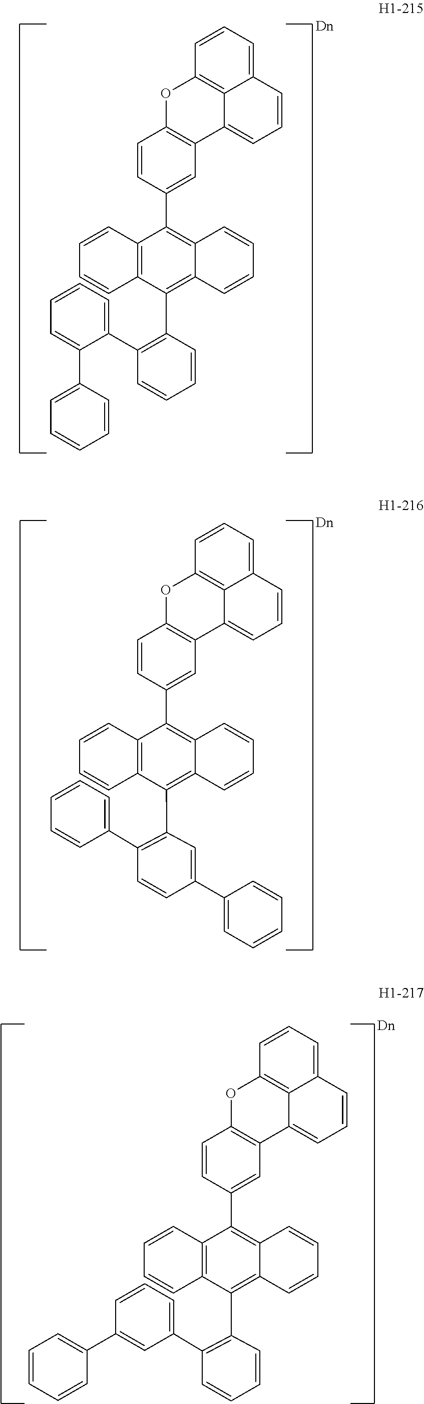

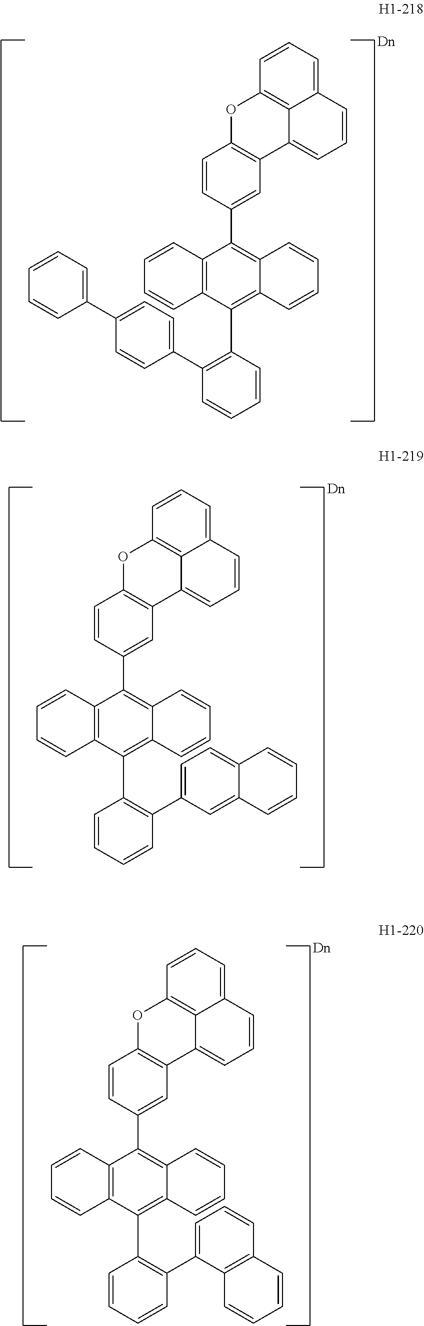

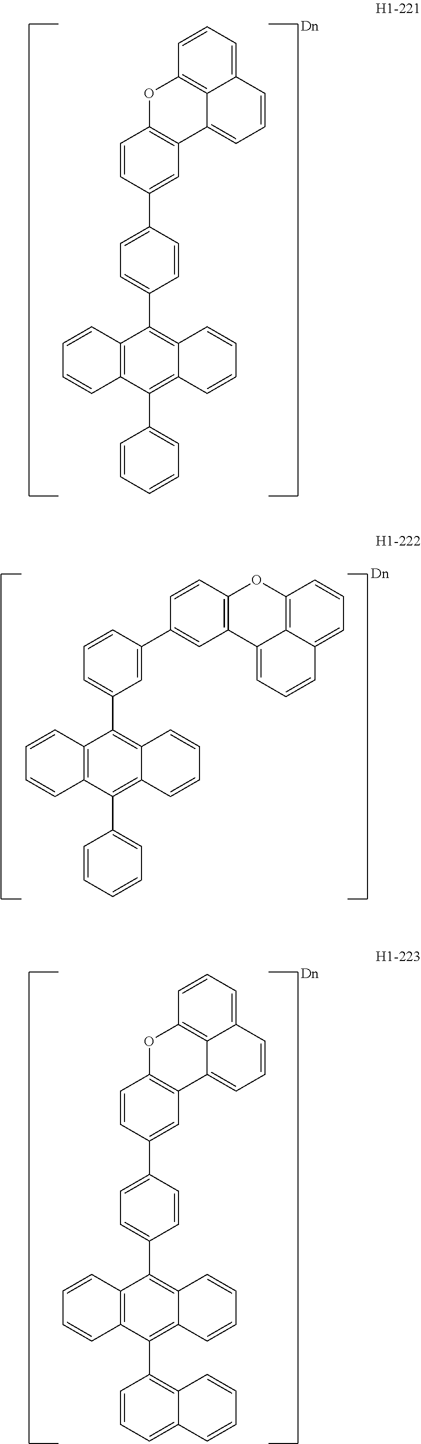

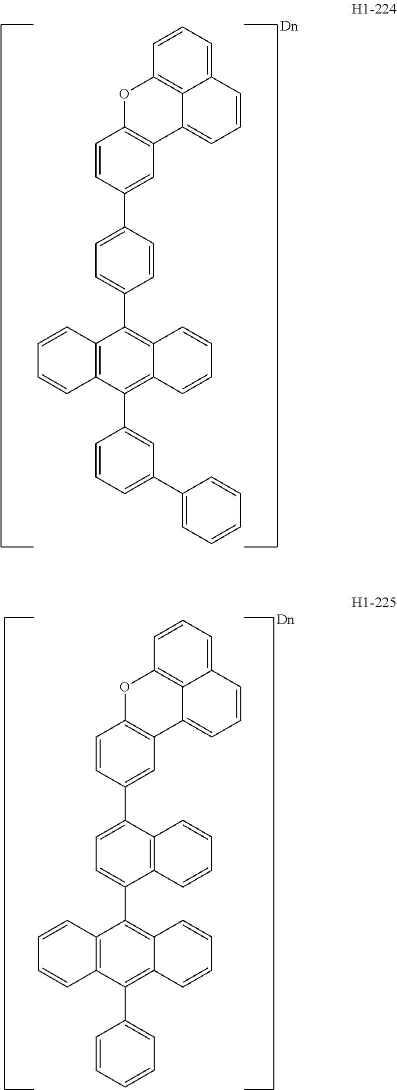

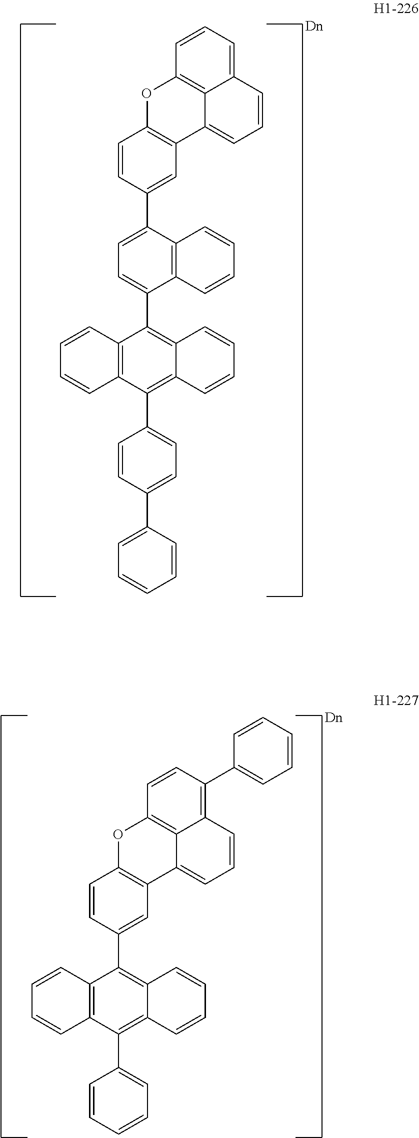

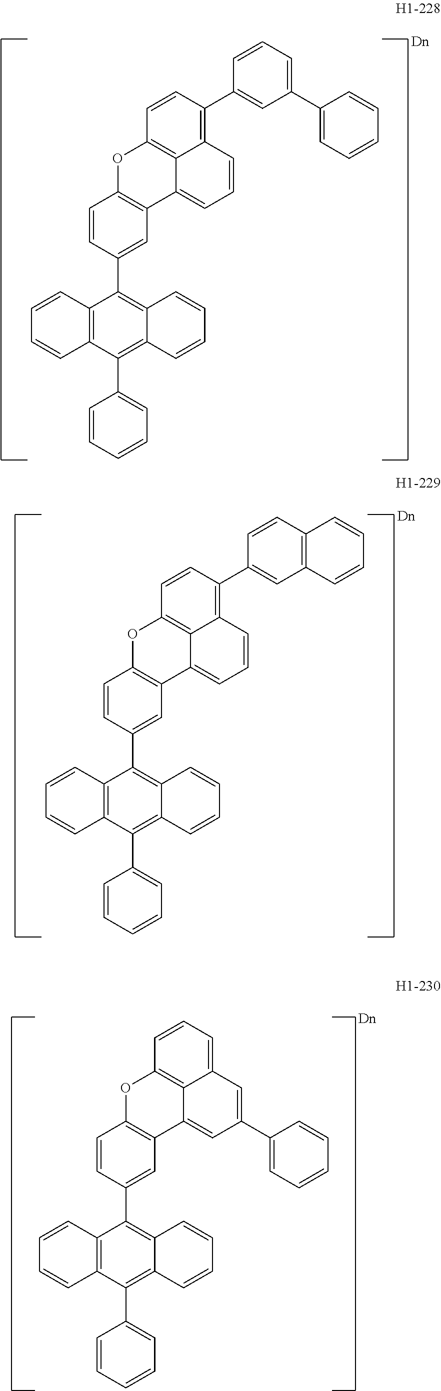

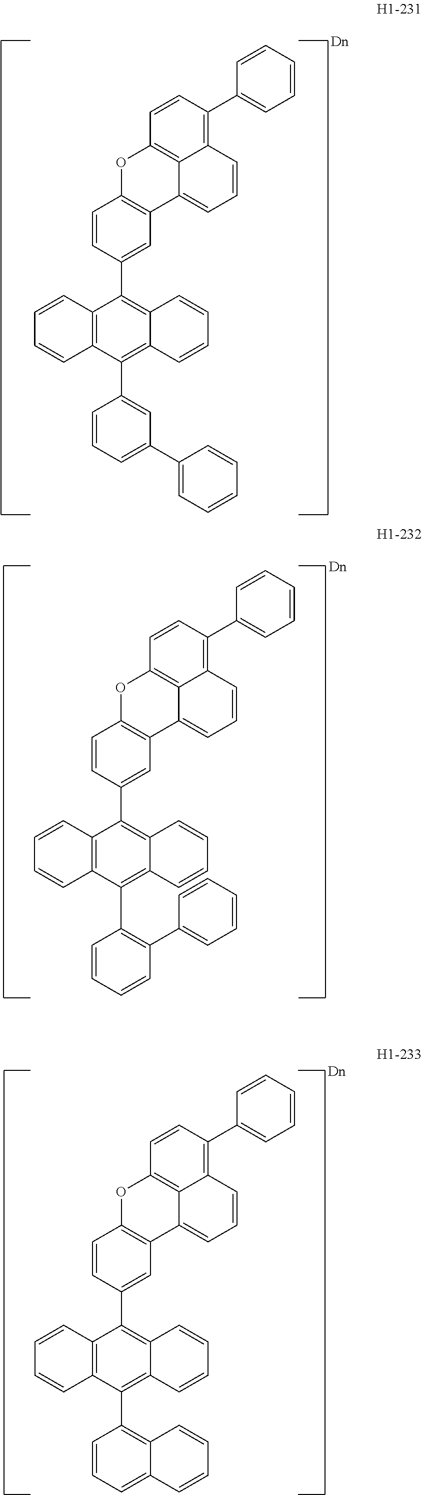

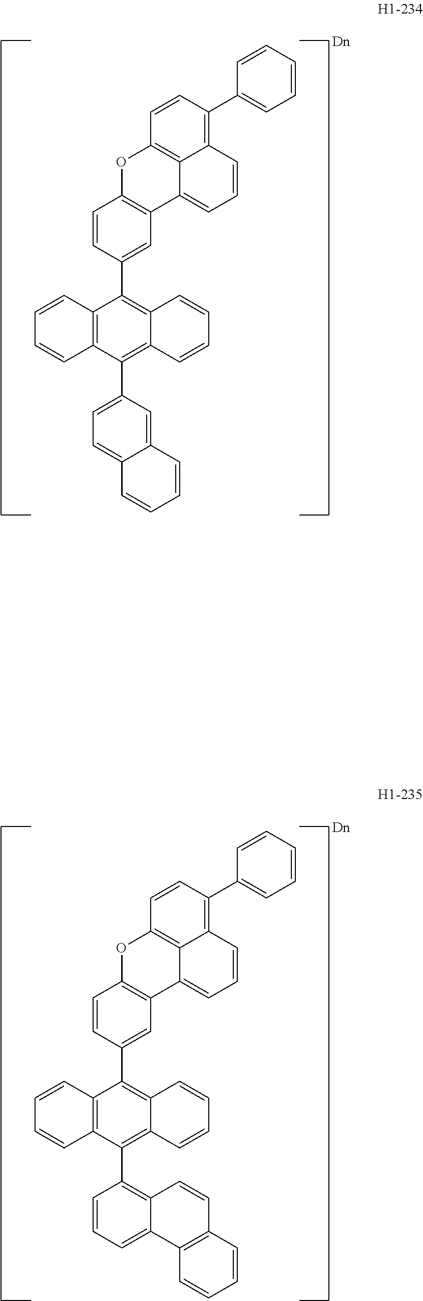

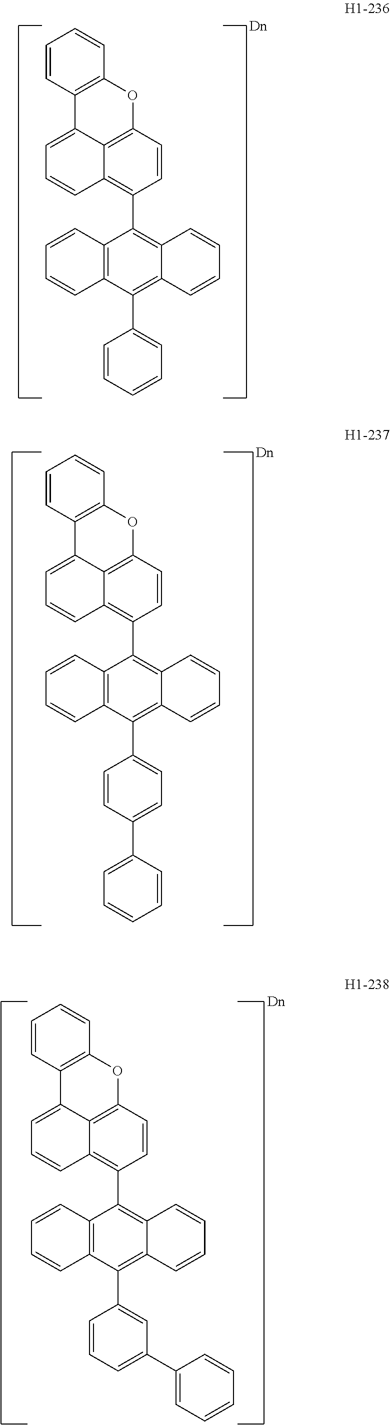

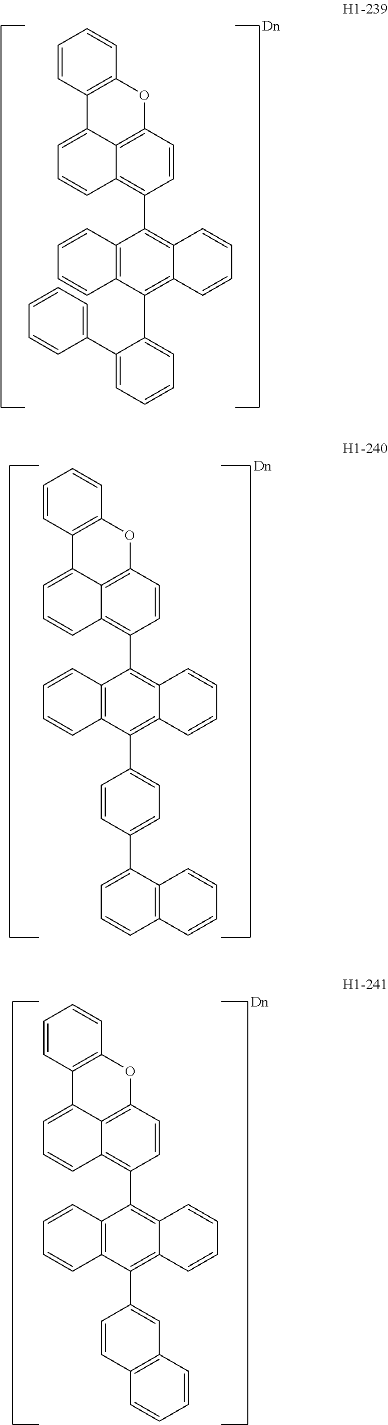

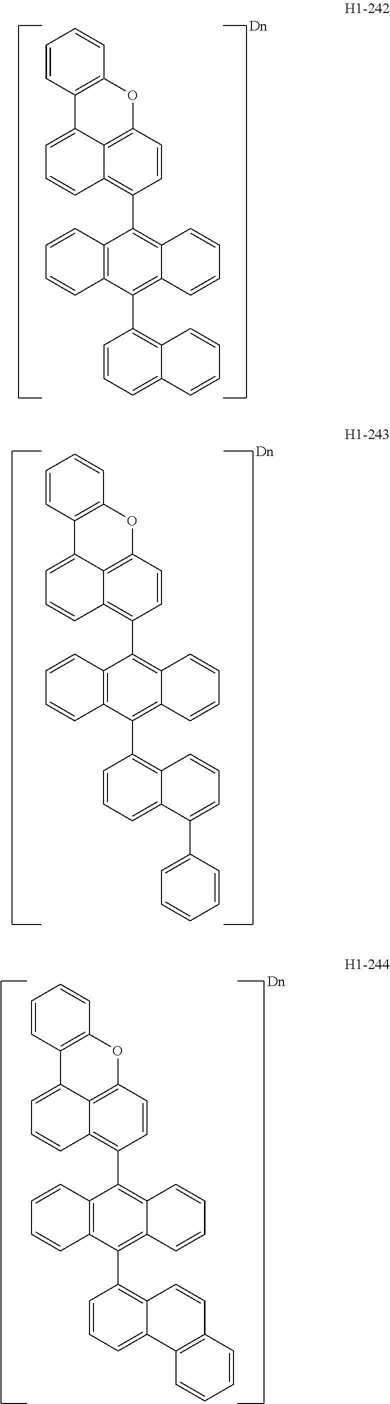

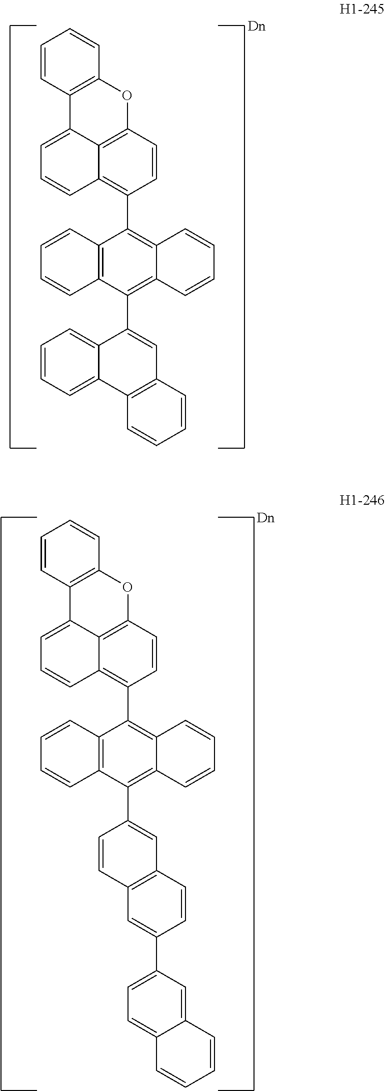

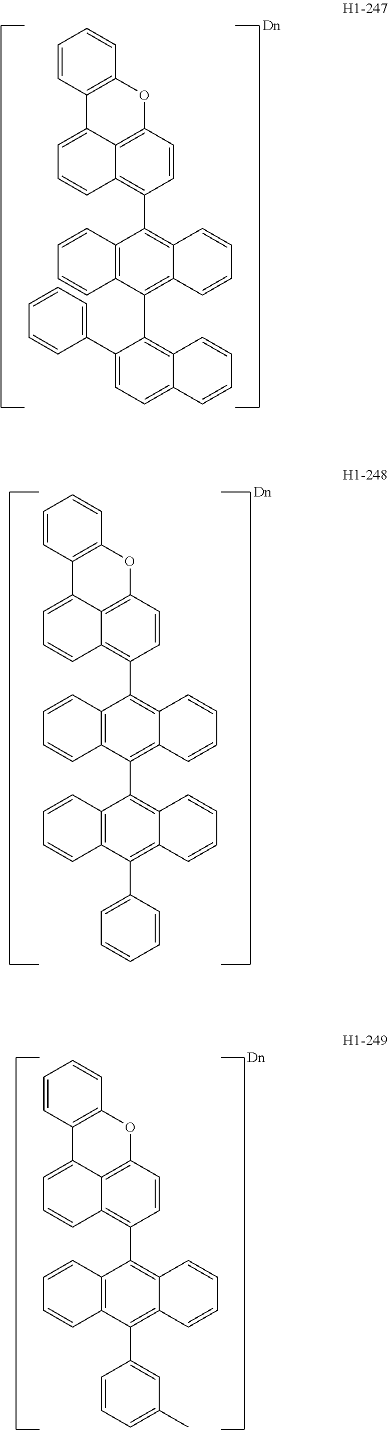

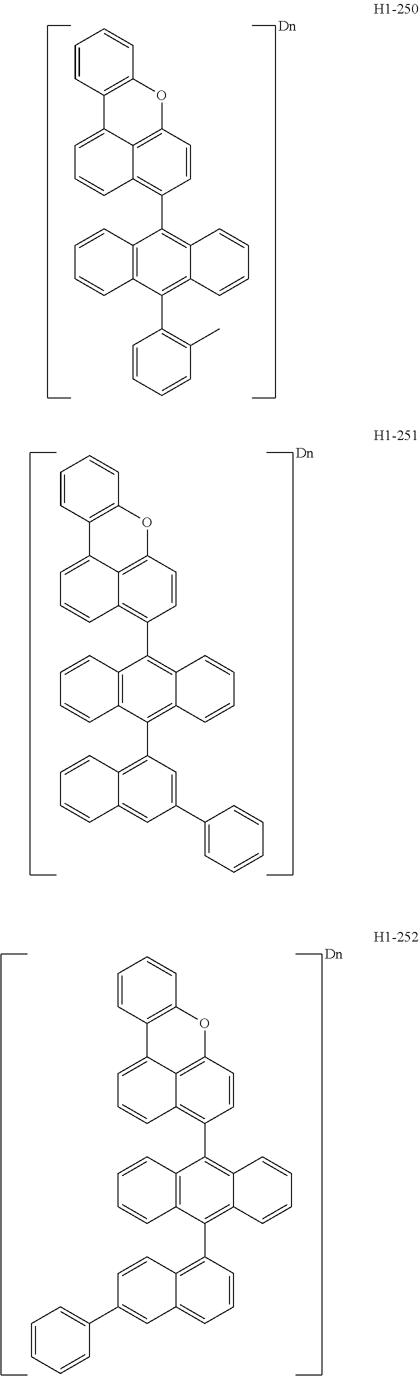

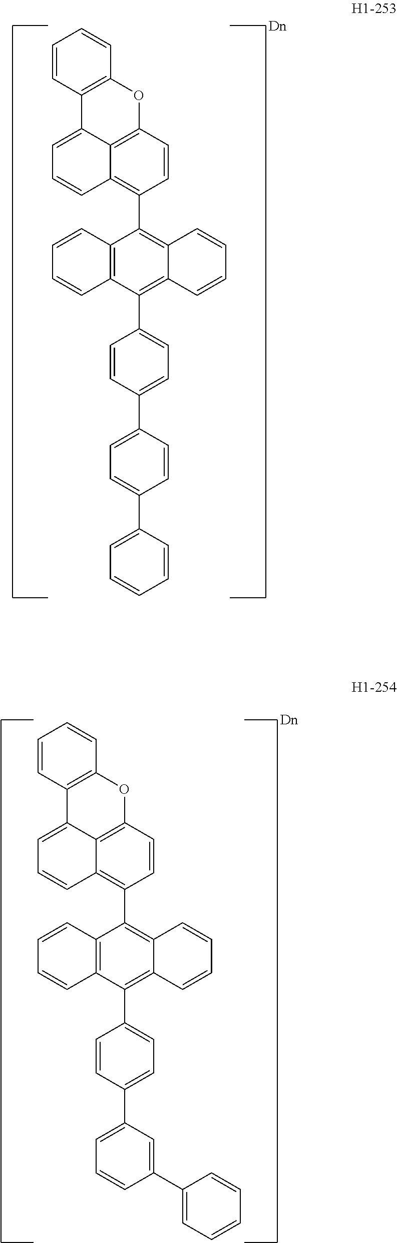

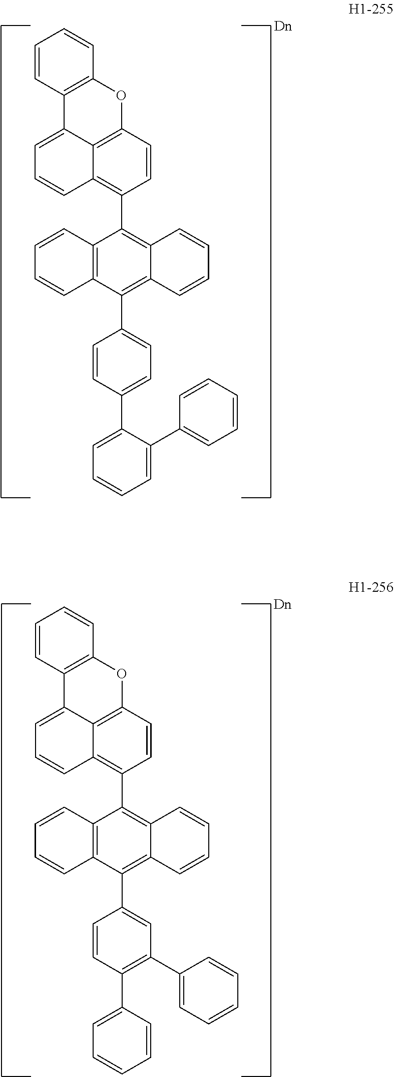

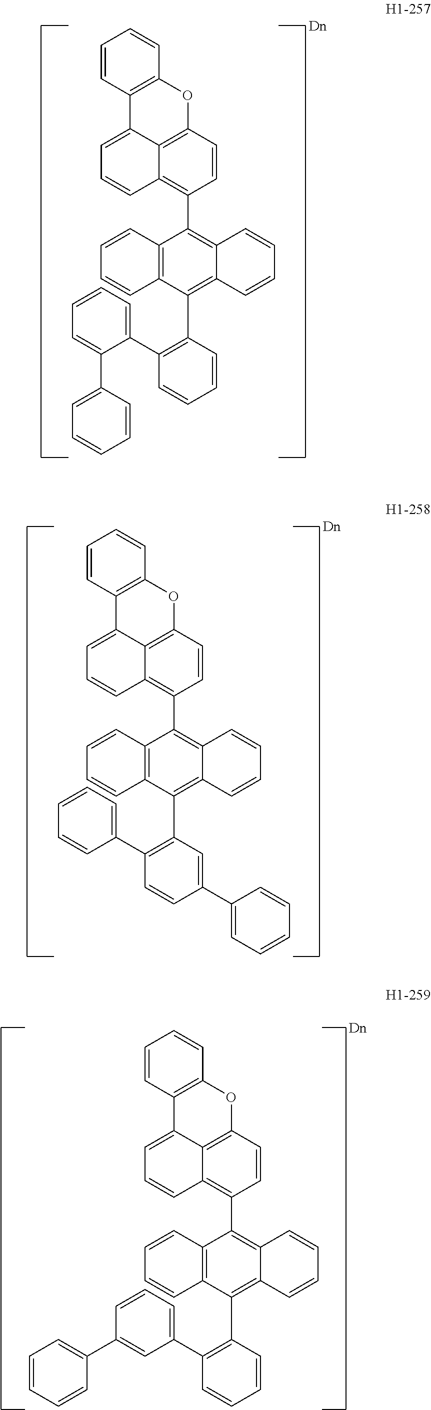

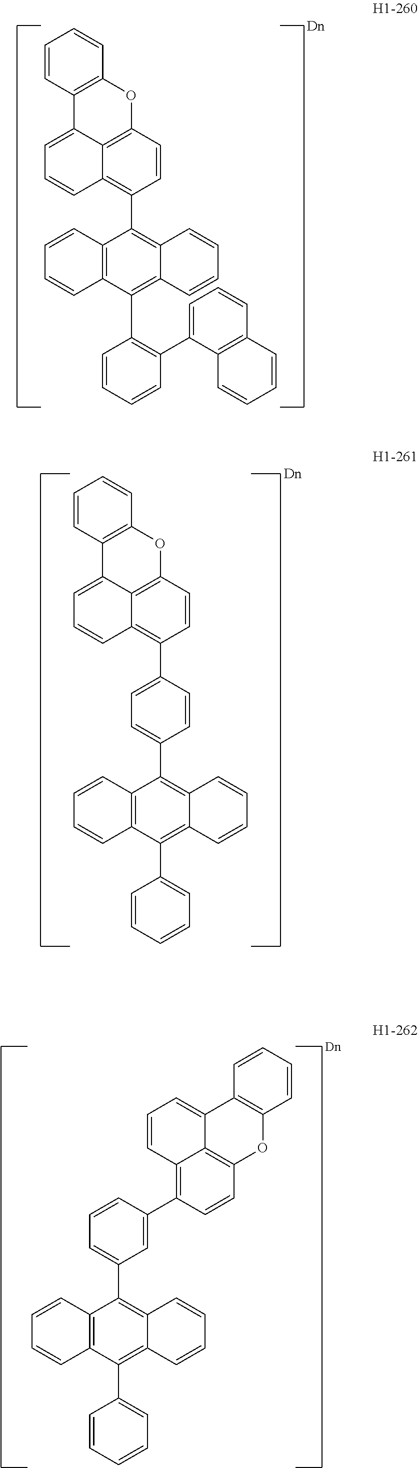

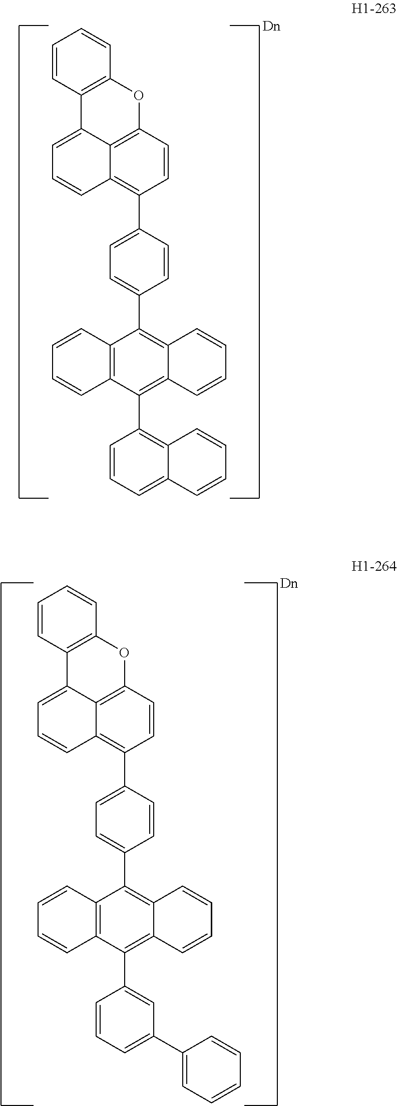

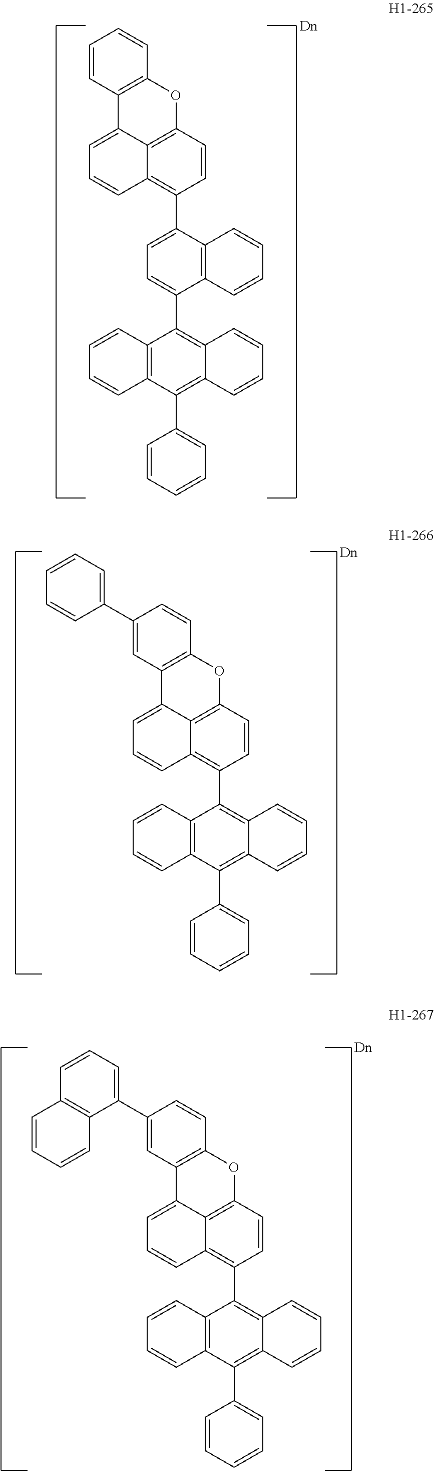

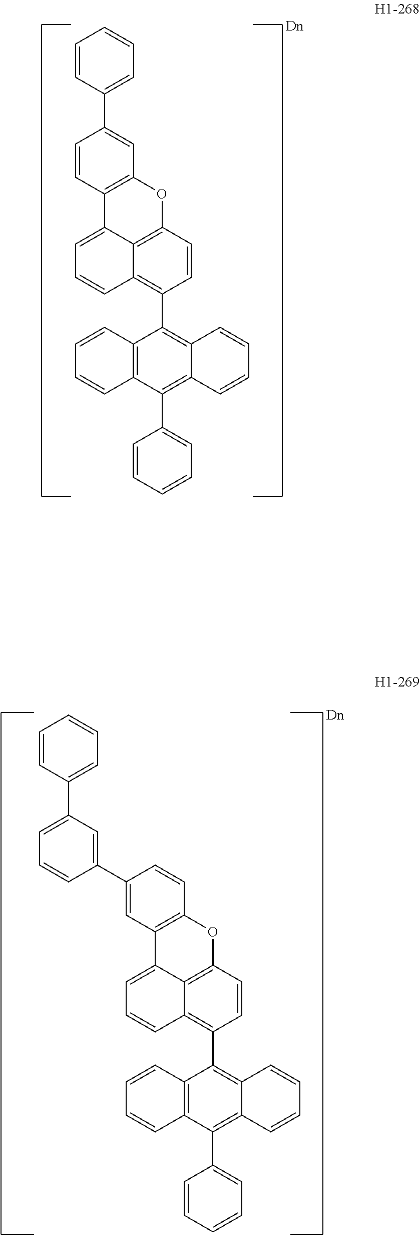

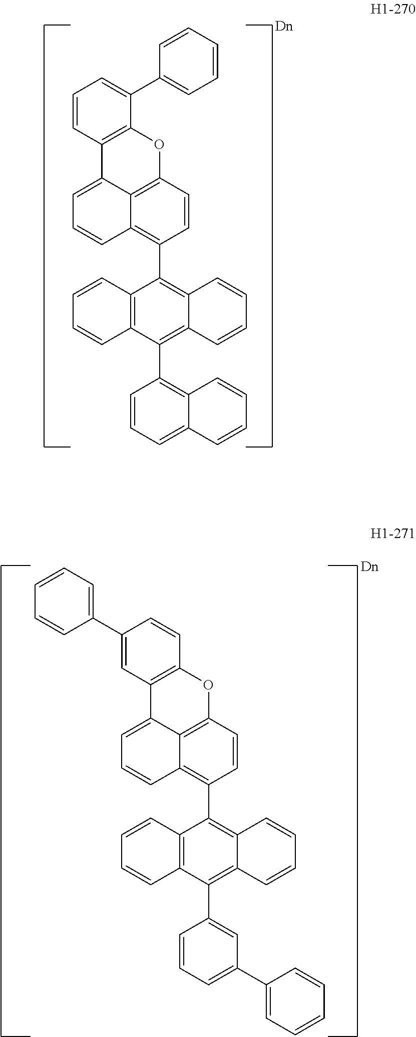

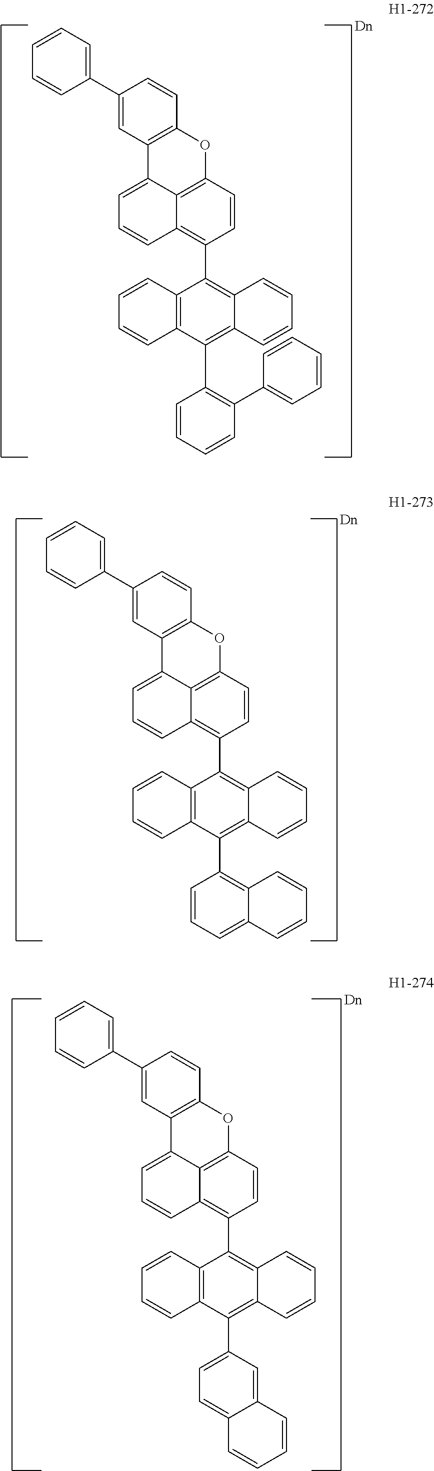

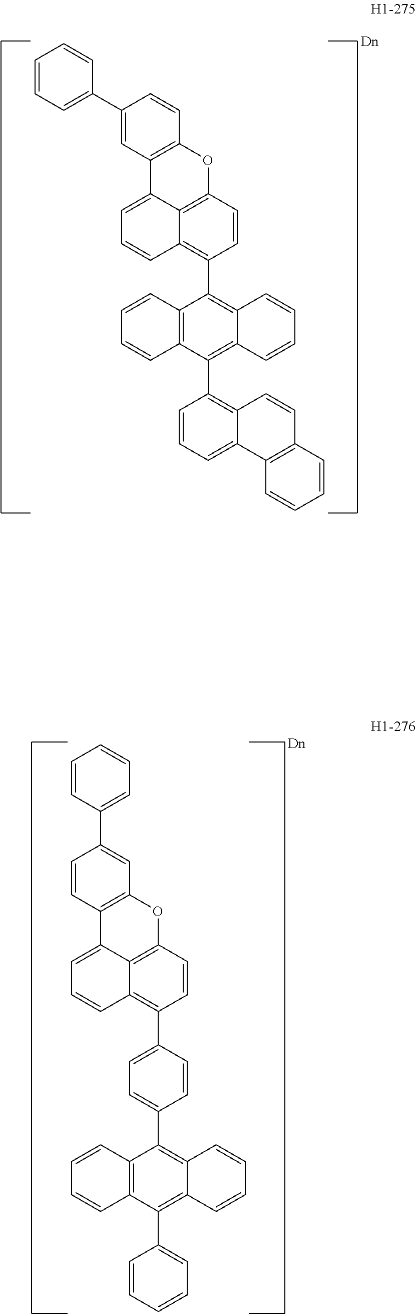

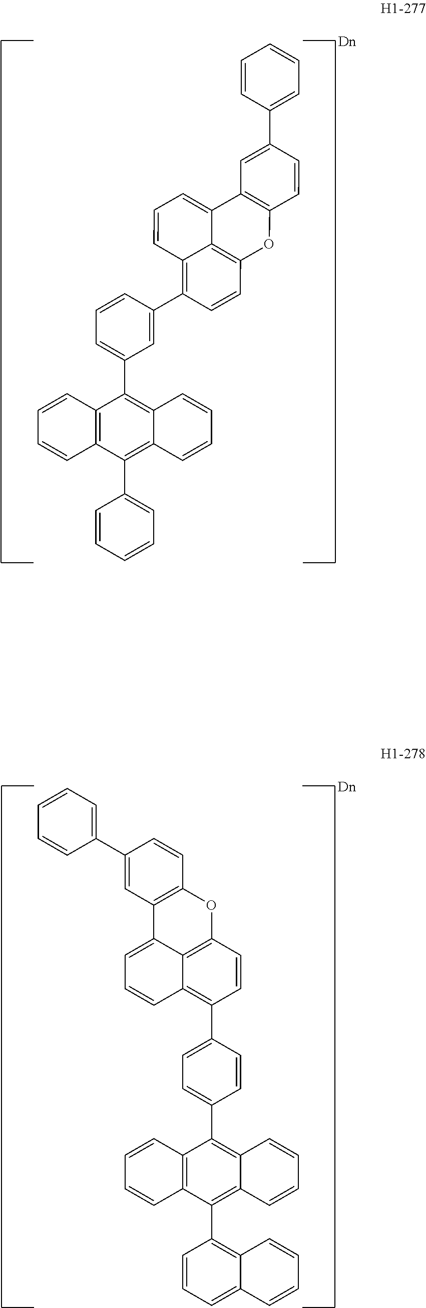

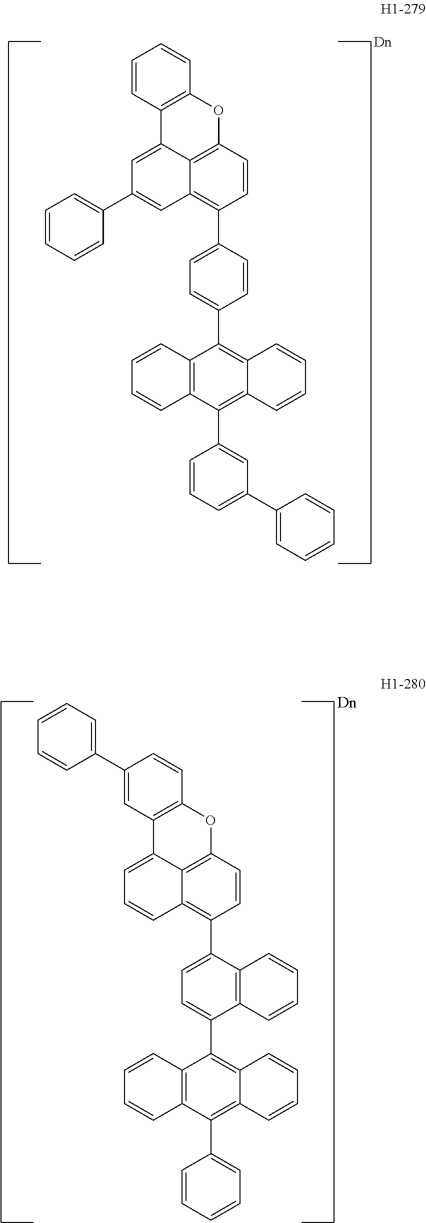

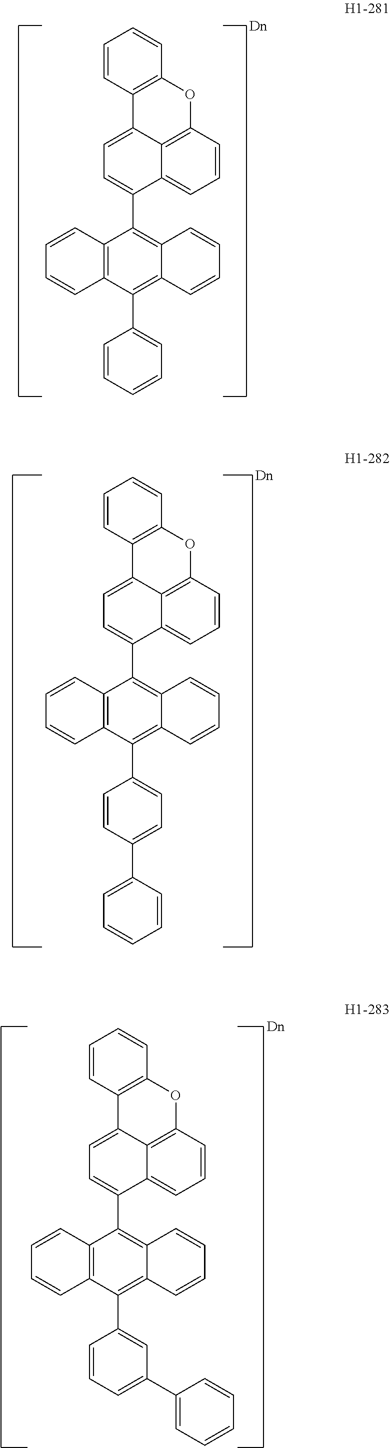

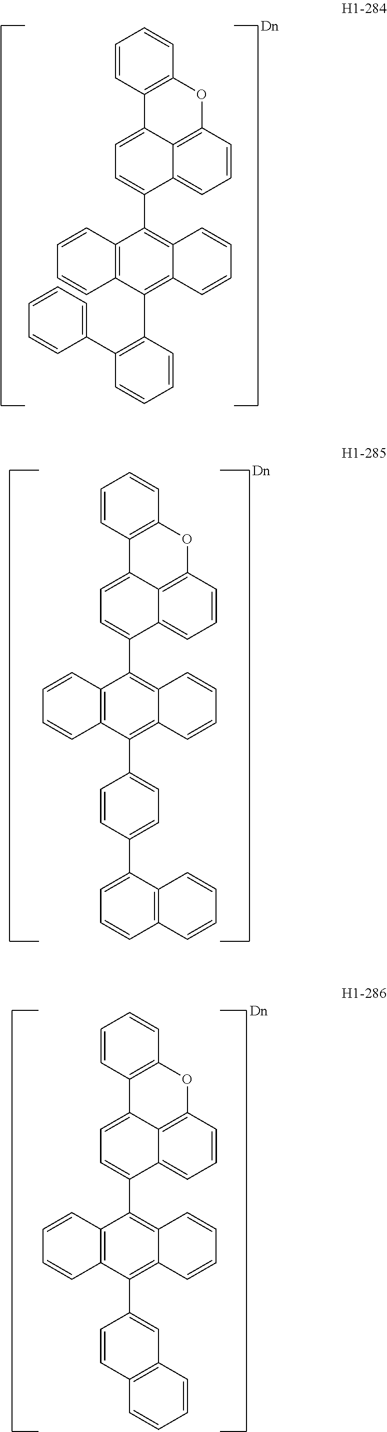

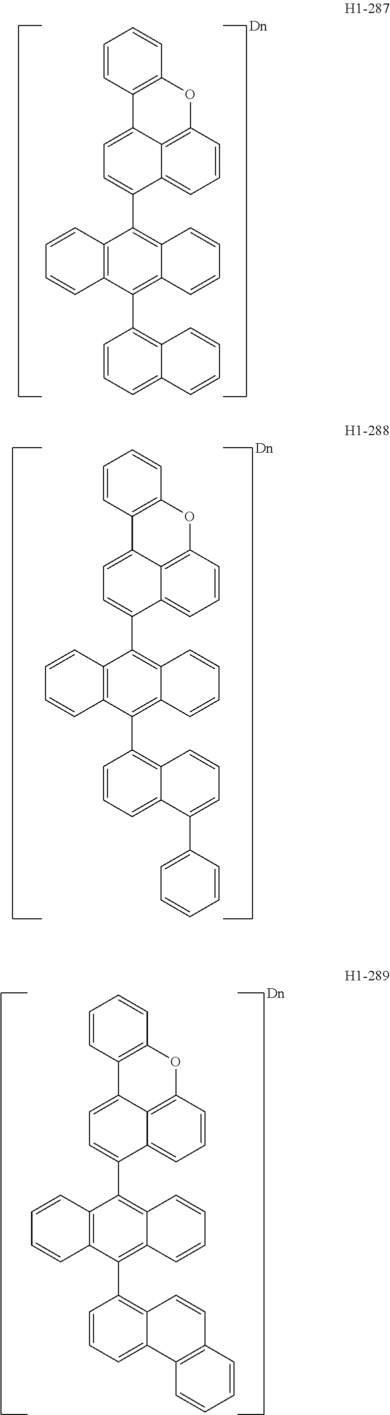

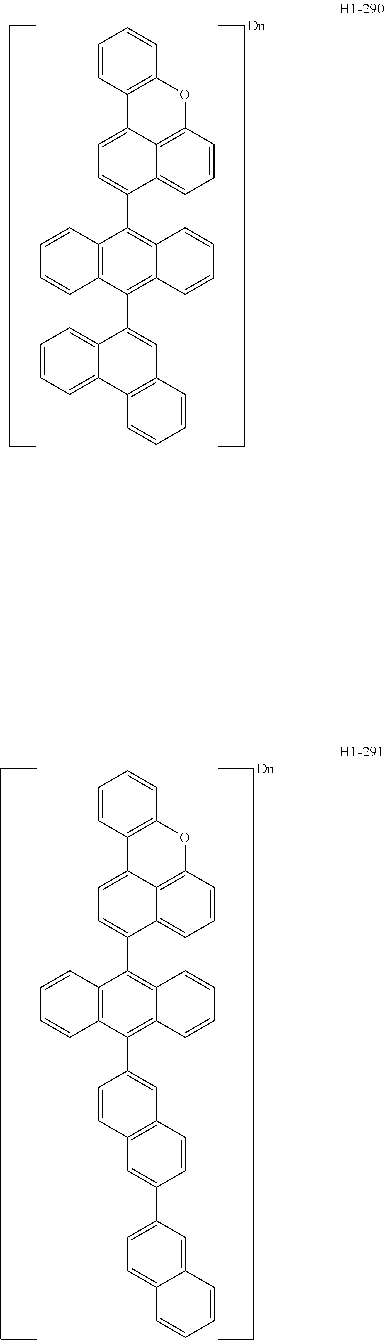

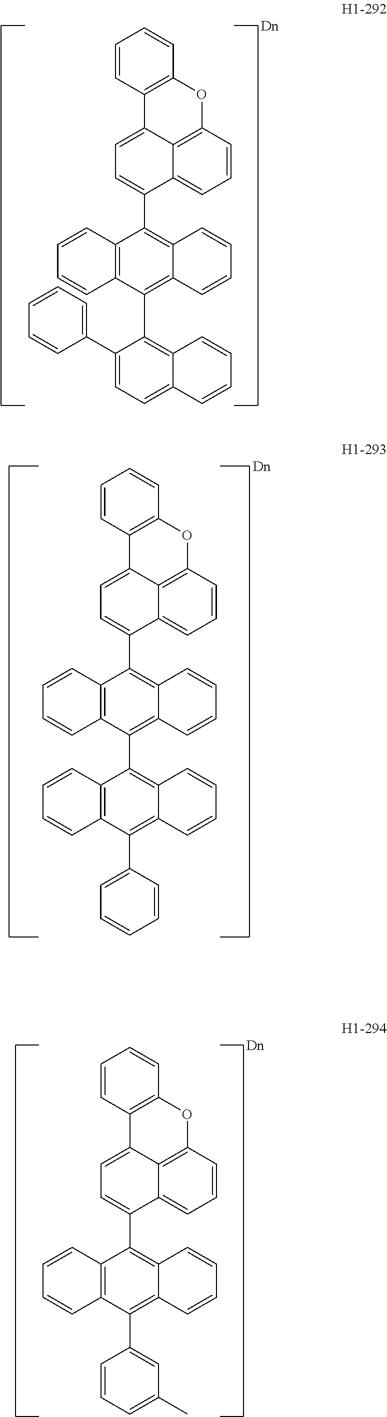

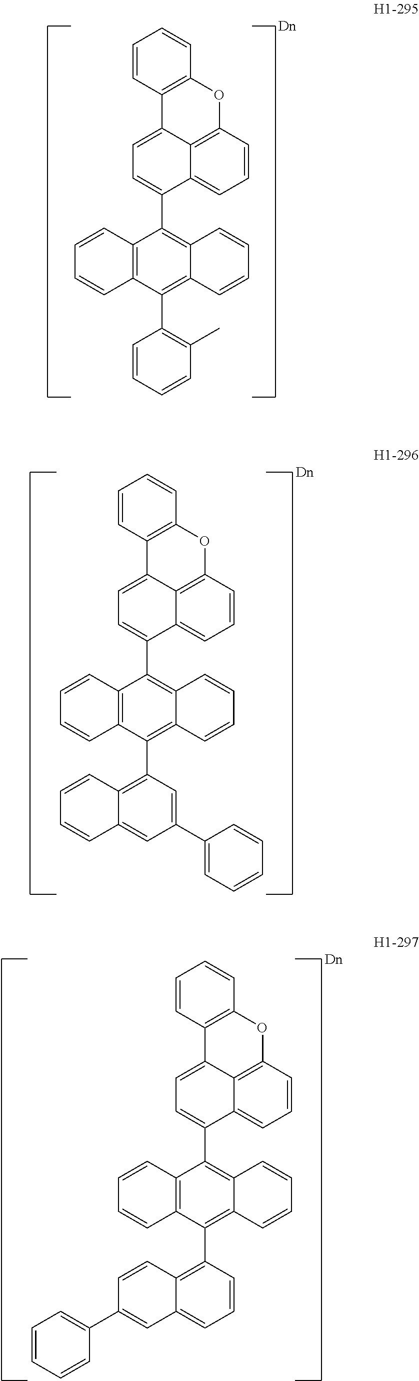

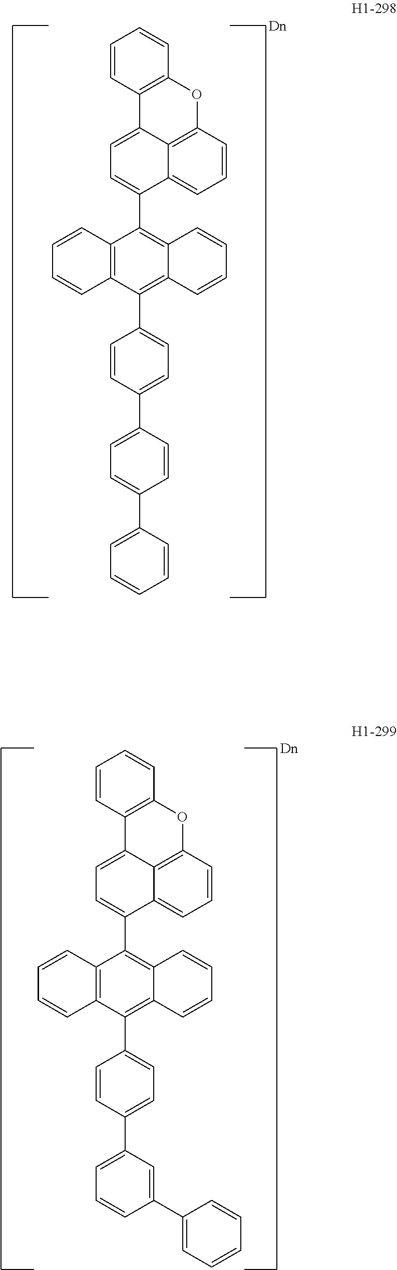

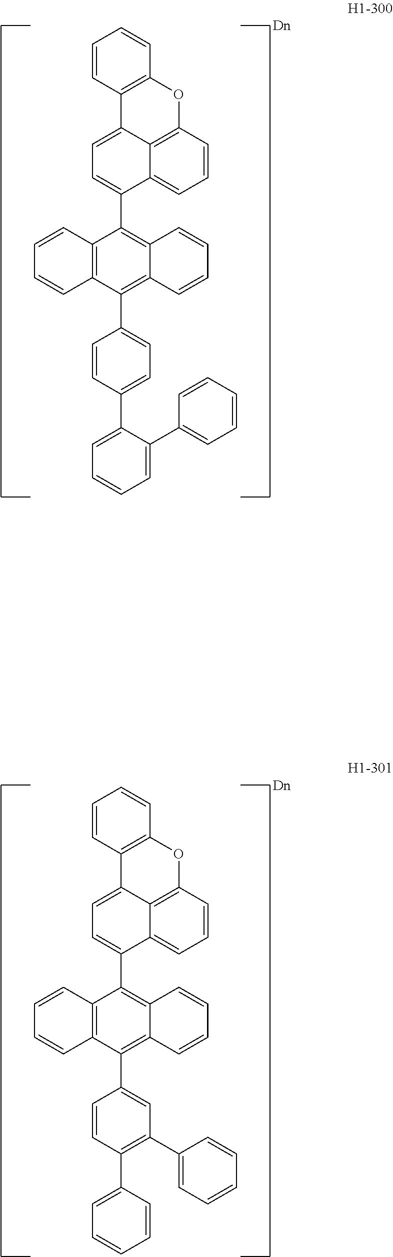

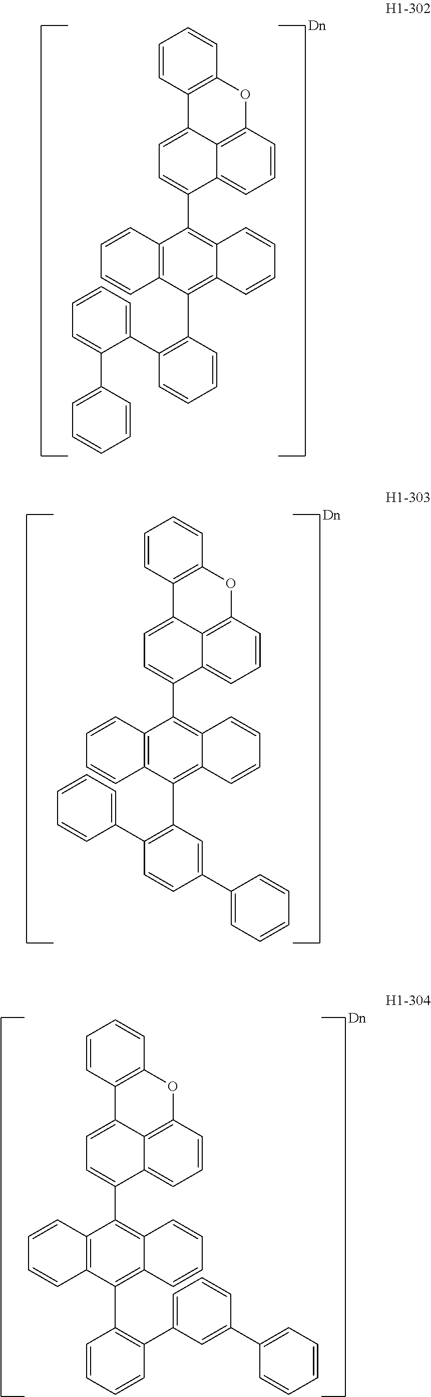

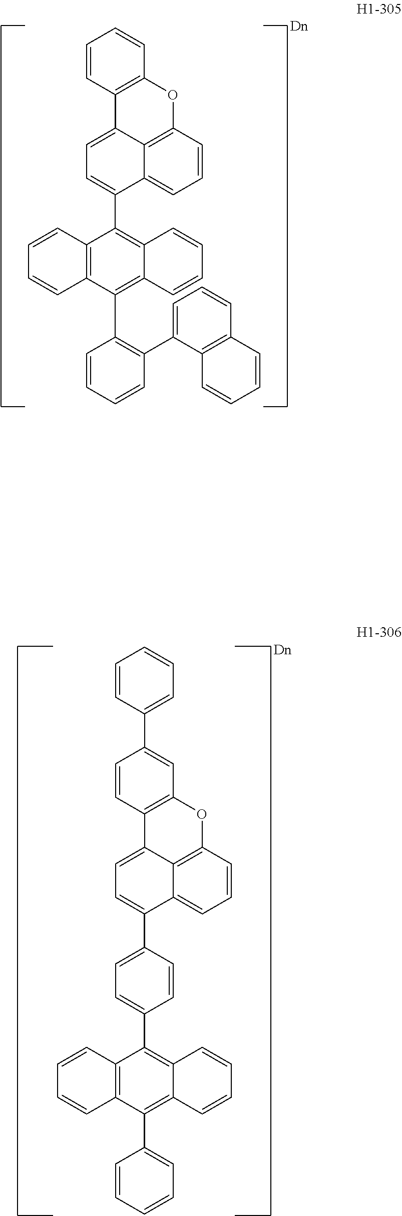

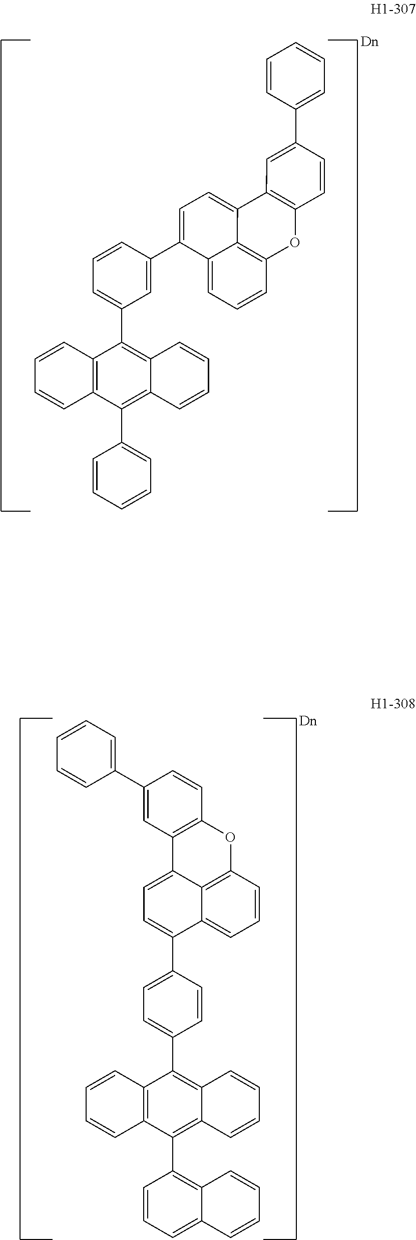

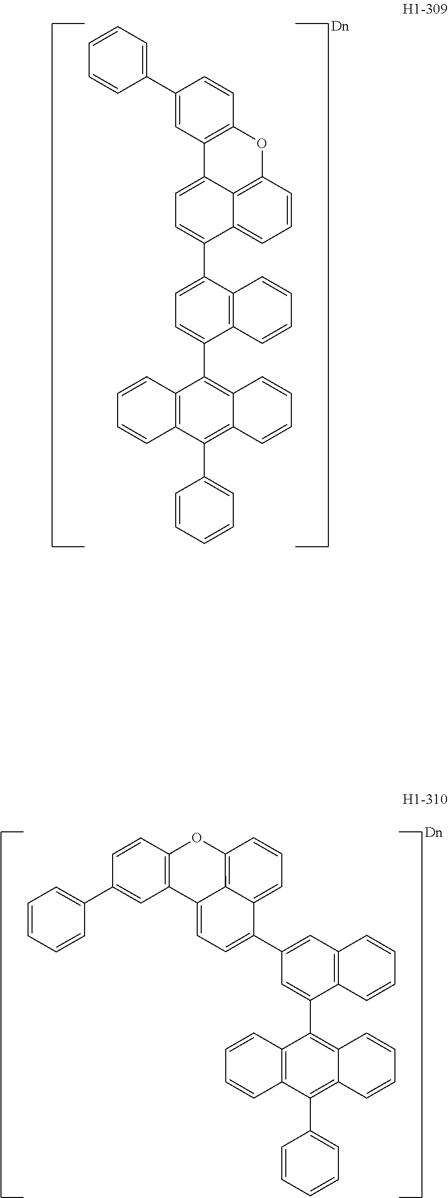

wherein “Do” in the above compounds means that n number of hydrogens is replaced by deuterium, wherein n represents an integer of 1 or more and the upper limit of n is determined by the number of hydrogens that can be substituted in each compound.

According to another embodiment, the present disclosure provides an organic electroluminescent compound represented by the following Formula 12.

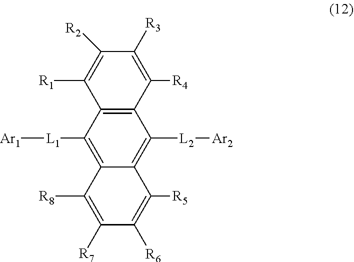

In Formula 12,
R₁ to R₈ each independently represent hydrogen or deuterium;
L₁ and L₂ each independently represent a single bond, a substituted or unsubstituted (C₆-C₃₀)arylene, or a substituted or unsubstituted (3- to 30-membered)heteroarylene; and
Ar₁ is represented by the following Formula 1-B or 1-C;

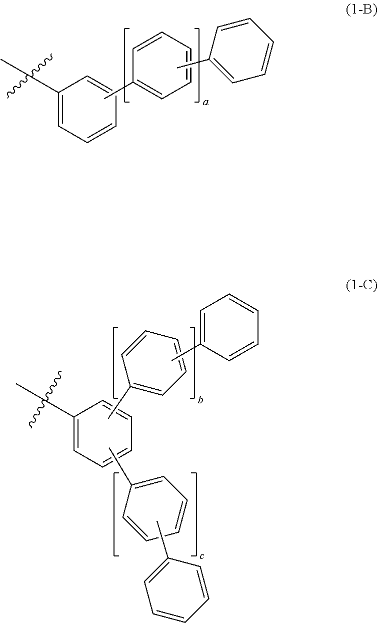

In Formulas 1-B and 1-C,
a represents an integer of 1 to 3, b and c each independently represent an integer of 0 to 3;
[symbol] represents a linking site to L₁; and
Ar₂ is represented by the following Formula 1-A;

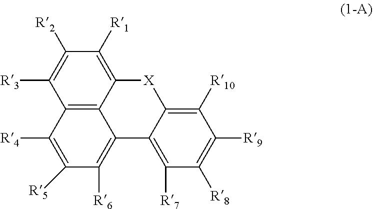

In Formula 1-A,
X represents —NR″—, —Se—, —O—, or —S—;
R′₁ to R′₁₀ each independently represent hydrogen, deuterium, a substituted or unsubstituted (C₆-C₃₀)aryl, or a substituted or unsubstituted (3- to 30-membered)heteroaryl; or may be linked to the adjacent substituents to form a substituted or unsubstituted (3- to 30-membered) mono- or polycyclic, alicyclic, aromatic ring, or a combination thereof; and
any one of R′₁ to R′₁₀ is linked to L₂.

According to one embodiment, the organic electroluminescent compound represented by Formula 12 above may be more specifically illustrated by the following compounds, but is not limited thereto.

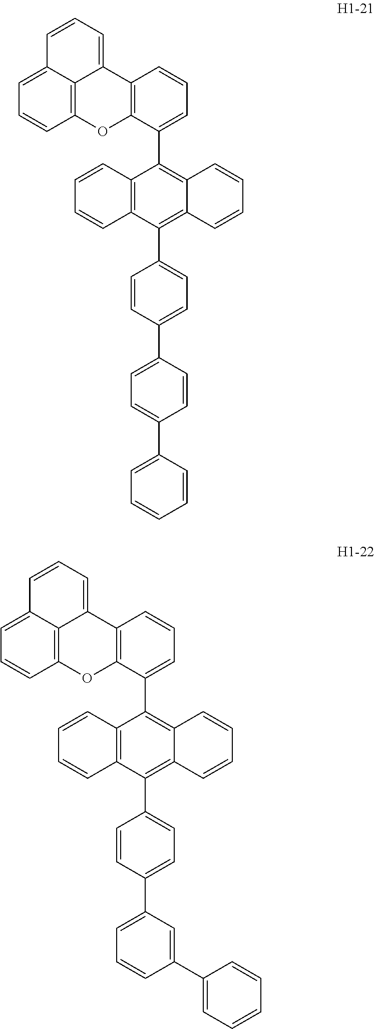 [SMILES: c1ccc(-c2ccc(-c3ccc(-c4c5ccccc5c(-c5cccc6c5Oc5cccc7cccc-6c57)c5ccccc45)cc3)cc2)cc1 ; c1ccc(-c2cccc(-c3ccc(-c4c5ccccc5c(-c5cccc6c5Oc5cccc7cccc-6c57)c5ccccc45)cc3)c2)cc1]

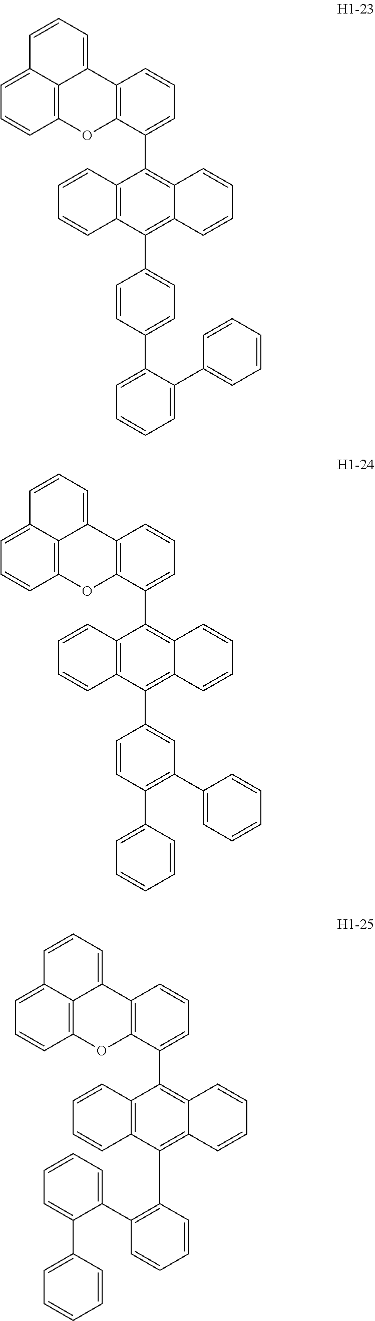 [SMILES: c1ccc(-c2ccccc2-c2ccc(-c3c4ccccc4c(-c4cccc5c4Oc4cccc6cccc-5c46)c4ccccc34)cc2)cc1 ; c1ccc(-c2ccc(-c3c4ccccc4c(-c4cccc5c4Oc4cccc6cccc-5c46)c4ccccc34)cc2-c2ccccc2)cc1 ; c1ccc(-c2ccccc2-c2ccccc2-c2c3ccccc3c(-c3cccc4c3Oc3cccc5cccc-4c35)c3ccccc23)cc1]

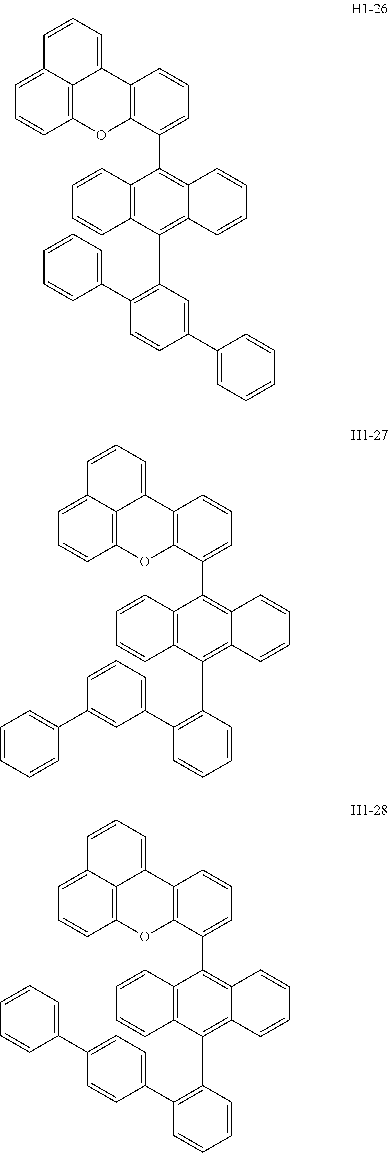 [SMILES: c1ccc(-c2ccc(-c3ccccc3)c(-c3c4ccccc4c(-c4cccc5c4Oc4cccc6cccc-5c46)c4ccccc34)c2)cc1 ; c1ccc(-c2cccc(-c3ccccc3-c3c4ccccc4c(-c4cccc5c4Oc4cccc6cccc-5c46)c4ccccc34)c2)cc1 ; c1ccc(-c2ccc(-c3ccccc3-c3c4ccccc4c(-c4cccc5c4Oc4cccc6cccc-5c46)c4ccccc34)cc2)cc1]

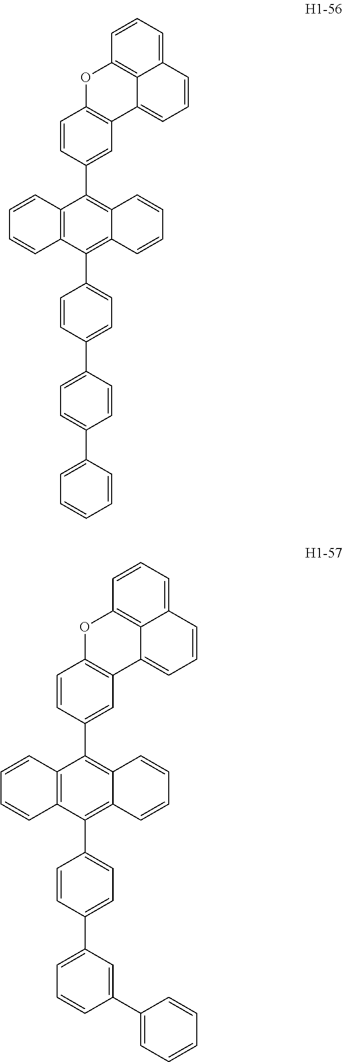 [SMILES: c1ccc(-c2ccc(-c3ccc(-c4c5ccccc5c(-c5ccc6c(c5)-c5cccc7cccc(c57)O6)c5ccccc45)cc3)cc2)cc1 ; c1ccc(-c2cccc(-c3ccc(-c4c5ccccc5c(-c5ccc6c(c5)-c5cccc7cccc(c57)O6)c5ccccc45)cc3)c2)cc1]

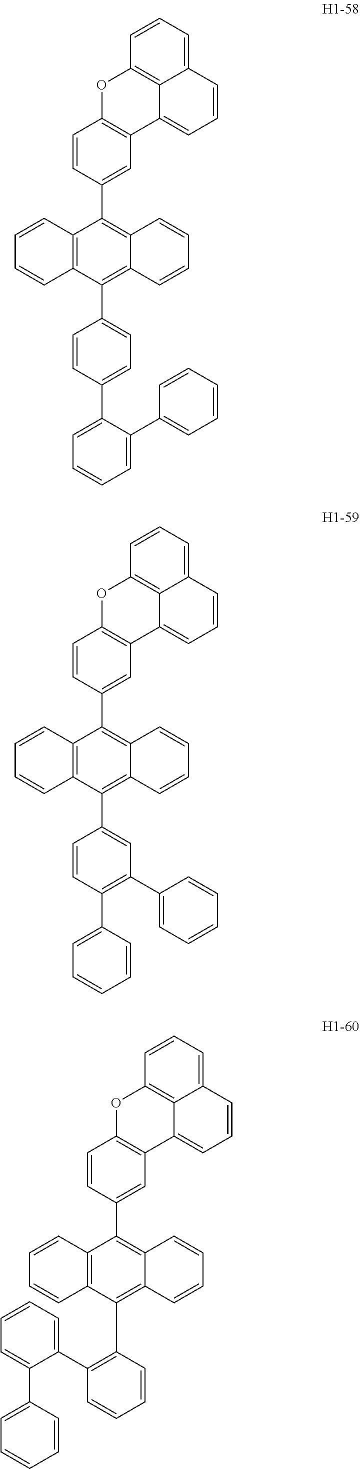 [SMILES: c1ccc(-c2ccccc2-c2ccc(-c3c4ccccc4c(-c4ccc5c(c4)-c4cccc6cccc(c46)O5)c4ccccc34)cc2)cc1 ; c1ccc(-c2ccc(-c3c4ccccc4c(-c4ccc5c(c4)-c4cccc6cccc(c46)O5)c4ccccc34)cc2-c2ccccc2)cc1 ; c1ccc(-c2ccccc2-c2ccccc2-c2c3ccccc3c(-c3ccc4c(c3)-c3cccc5cccc(c35)O4)c3ccccc23)cc1]

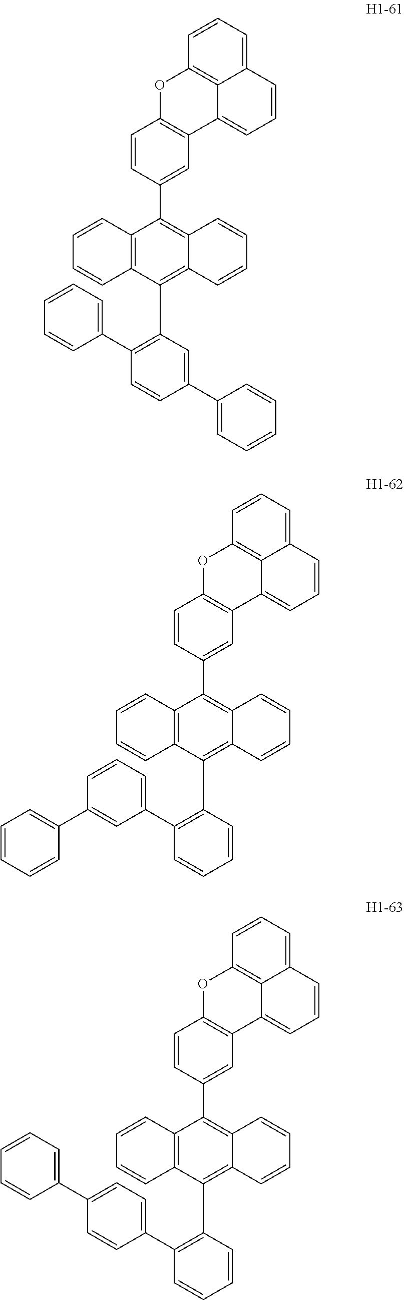

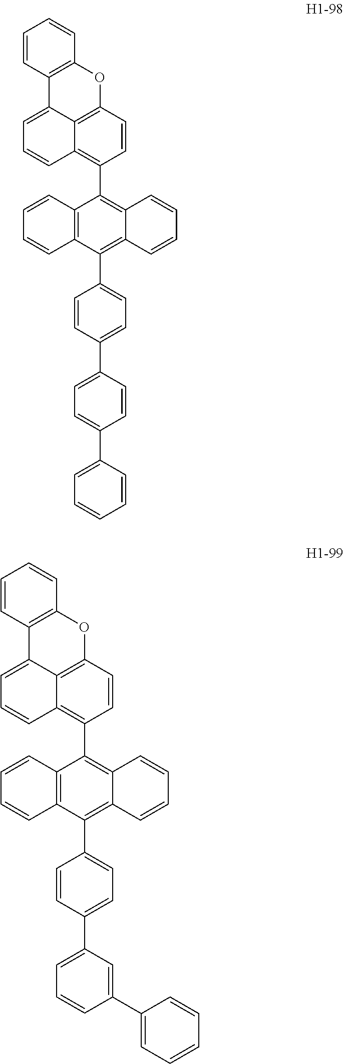 [SMILES: c1ccc(-c2ccc(-c3ccc(-c4c5ccccc5c(-c5ccc6c7c(cccc57)-c5ccccc5O6)c5ccccc45)cc3)cc2)cc1 ; c1ccc(-c2cccc(-c3ccc(-c4c5ccccc5c(-c5ccc6c7c(cccc57)-c5ccccc5O6)c5ccccc45)cc3)c2)cc1]

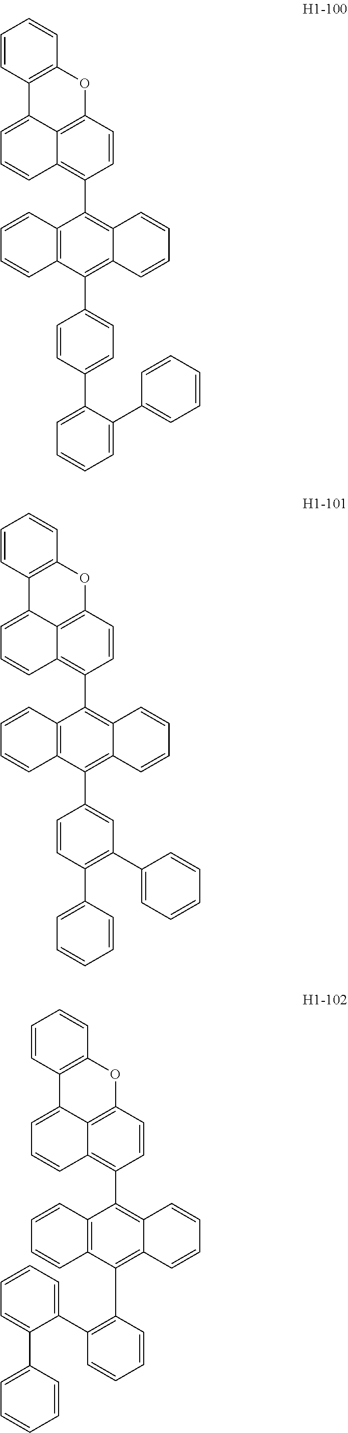 [SMILES: c1ccc(-c2ccccc2-c2ccc(-c3c4ccccc4c(-c4ccc5c6c(cccc46)-c4ccccc4O5)c4ccccc34)cc2)cc1 ; c1ccc(-c2ccc(-c3c4ccccc4c(-c4ccc5c6c(cccc46)-c4ccccc4O5)c4ccccc34)cc2-c2ccccc2)cc1 ; c1ccc(-c2ccccc2-c2ccccc2-c2c3ccccc3c(-c3ccc4c5c(cccc35)-c3ccccc3O4)c3ccccc23)cc1]

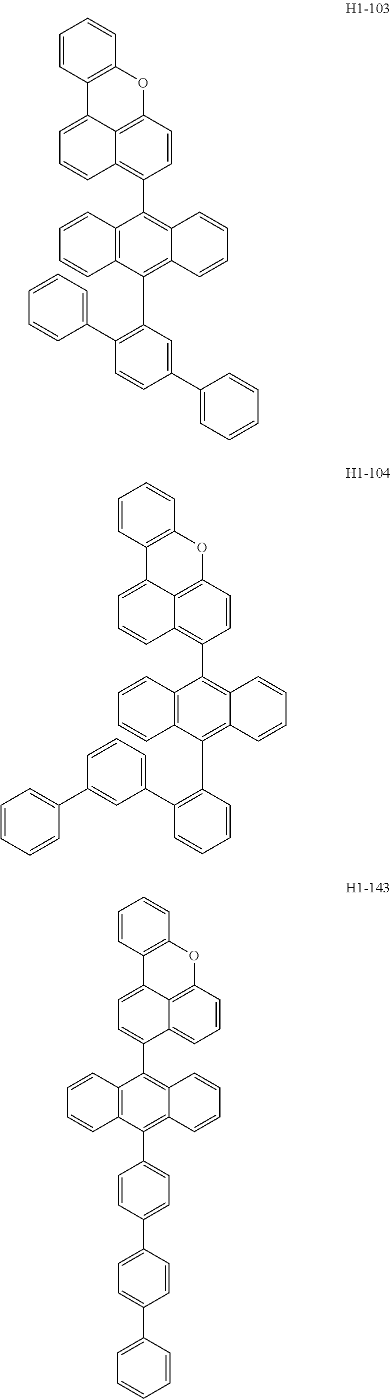 [SMILES: c1ccc(-c2ccc(-c3ccccc3)c(-c3c4ccccc4c(-c4ccc5c6c(cccc46)-c4ccccc4O5)c4ccccc34)c2)cc1 ; c1ccc(-c2cccc(-c3ccccc3-c3c4ccccc4c(-c4ccc5c6c(cccc46)-c4ccccc4O5)c4ccccc34)c2)cc1 ; c1ccc(-c2ccc(-c3ccc(-c4c5ccccc5c(-c5ccc6c7c(cccc57)Oc5ccccc5-6)c5ccccc45)cc3)cc2)cc1]

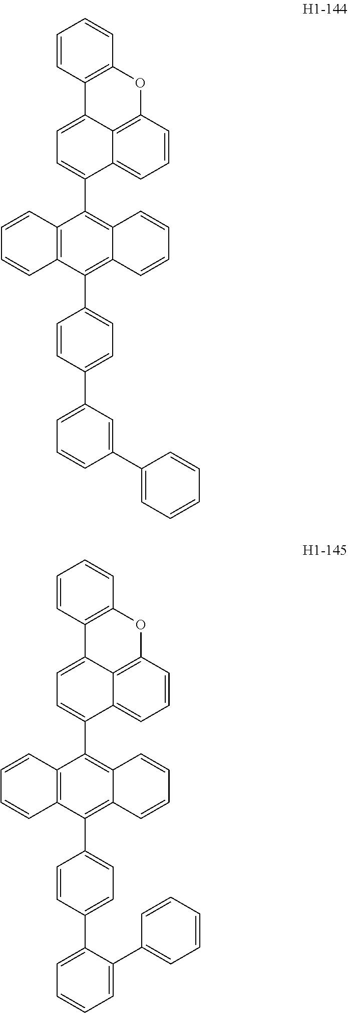 [SMILES: c1ccc(-c2cccc(-c3ccc(-c4c5ccccc5c(-c5ccc6c7c(cccc57)Oc5ccccc5-6)c5ccccc45)cc3)c2)cc1 ; c1ccc(-c2ccccc2-c2ccc(-c3c4ccccc4c(-c4ccc5c6c(cccc46)Oc4ccccc4-5)c4ccccc34)cc2)cc1]

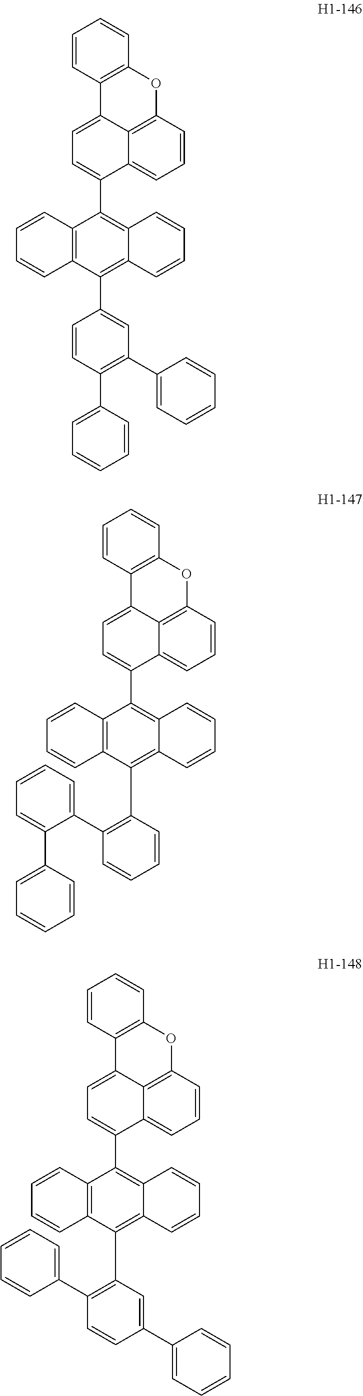 [SMILES: c1ccc(-c2ccc(-c3c4ccccc4c(-c4ccc5c6c(cccc46)Oc4ccccc4-5)c4ccccc34)cc2-c2ccccc2)cc1 ; c1ccc(-c2ccccc2-c2ccccc2-c2c3ccccc3c(-c3ccc4c5c(cccc35)Oc3ccccc3-4)c3ccccc23)cc1 ; c1ccc(-c2ccc(-c3ccccc3)c(-c3c4ccccc4c(-c4ccc5c6c(cccc46)Oc4ccccc4-5)c4ccccc34)c2)cc1]

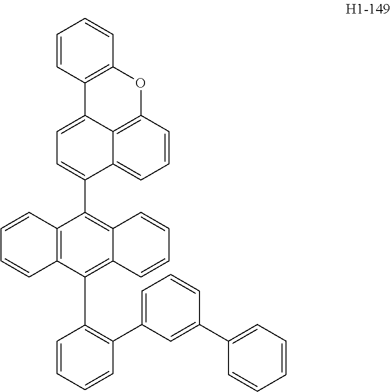 [SMILES: c1ccc(-c2cccc(-c3ccccc3-c3c4ccccc4c(-c4ccc5c6c(cccc46)Oc4ccccc4-5)c4ccccc34)c2)cc1]

Hereinafter, an organic electroluminescent device will be described to which the aforementioned plurality of host materials and/or organic electroluminescent compound is (are) applied.

The organic electroluminescent device according to one embodiment includes a first electrode; a second electrode; and at least one organic layer(s) interposed between the first electrode and the second electrode. The organic layer may include a light-emitting layer, and the light-emitting layer may comprise a plurality of host materials comprising at least one first host compound represented by Formula 1 and at least one second host compound represented by Formula 2, wherein the first host compound and second host compound are different from each other.

According to one embodiment, the plurality of host materials of the present disclosure comprises at least one of compounds H1-1 to H1-310 which is(are) a first host compound, and at least one of compounds H2-1 to H2-366, which is(are) a second host compound. The plurality of host materials may be included in the same organic layer, for example, the same light-emitting layer, or may be included in different light-emitting layers.

According to one embodiment, the light-emitting layer may comprise an organic electroluminescent compound represented by Formula 11 or an organic electroluminescent compound represented by Formula 12 as a host material. For example, the organic electroluminescent device according to the present disclosure may comprise at least one of compounds H1-156 to H1-310, or at least one of compounds H1-21 to H1-28, H1-56 to H1-63, H1-98 to H1-104, and H1-143 to H1-149 as a host material in a light-emitting layer.

The organic layer may further comprise at least one layer selected from a hole injection layer, a hole transport layer, a hole auxiliary layer, a light-emitting auxiliary layer, an electron transport layer, an electron injection layer, an interlayer, a hole-blocking layer, an electron-blocking layer and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron-blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron-blocking material. Further, the electron transport layer, the electron injection layer, the electron buffer layer, or the hole-blocking layer may contain the azine-based compound as an electron transport material, an electron injection material, an electron buffer material, or a hole-blocking material. Further, the organic layer may further comprise at least one metal selected from the group consisting of metals from Group 1, metals from Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has suggested various structures such as a parallel side-by-side arrangement method, a stacking arrangement method, CCM (color conversion material) method, etc., according to the arrangement of R (red), G (green), YG (yellowish green), or B (blue) light-emitting units. In addition, the plurality of host materials and/or the organic electroluminescent material according to one embodiment may also be applied to the organic electroluminescent device comprising a QD (quantum dot).

One of the first electrode and the second electrode may be an anode and the other may be a cathode, wherein the first electrode and the second electrode may each be formed as a transmissive conductive material, a transflective conductive material, or a reflective conductive material. The organic electroluminescent device may be a top emission type, a bottom emission type, or a dual-side emission type according to the kinds of the material forming the first electrode and the second electrode.

A hole injection layer, a hole transport layer, an electron-blocking layer, or a combination thereof can be used between the anode and the light-emitting layer. The hole injection layer may be multi-layered in order to lower the hole injection barrier (or hole injection voltage) from the anode to the hole transport layer or the electron-blocking layer, wherein each of the multi-layers may use two compounds simultaneously. Also, the hole injection layer may be doped as a p-dopant. Also, the electron-blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron-blocking layer may be multi-layered, and wherein each layer may use a plurality of compounds.

An electron buffer layer, a hole-blocking layer, an electron transport layer, an electron injection layer, or a combination thereof can be used between the light-emitting layer and the cathode. The electron buffer layer may be multi-layered in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layered, wherein each layer may use a plurality of compounds. Further, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron-blocking layer. The light-emitting auxiliary layer, the hole auxiliary layer, or the electron-blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤x≤2), AlOₓ(1≤x≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light emitting units, for example, a plurality of three or more light-emitting units, having first and second electrodes opposed to each other on a substrate and a light emitting layer that is stacked between the first and second electrodes and emits light in a specific wavelength range. According to one embodiment, the organic electroluminescent device may include a plurality of light-emitting units, and each of the light-emitting units may include a hole transport band, a light-emitting layer, and an electron transport band, and the hole transport band may include a hole injection layer and a hole transport layer, and the electron transfer zone may include an electron transport layer and an electron injection layer. According to one embodiment, three or more light emitting layers may be included in the light emitting unit. A plurality of light emitting units may emit the same color or different colors. Additionally, one light emitting unit may include one or more light emitting layers, and the plurality of light emitting layers may be light emitting layers of the same or different colors. It may include one or more charge generation layers located between each light-emitting unit. The charge generation layer refers to the layer in which holes and electrons are generated when voltage is applied. When there are three or more light-emitting units, a charge generation layer may be located between each light-emitting unit. At this time, the plurality of charge generation layers may be the same or different from each other. By disposing the charge generation layer between light-emitting units, current efficiency is increased in each light-emitting unit and charges can be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an n-type charge generation layer and a p-type charge generation layer, and the n-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The p-type charge generation layer may be made of a metal or an organic material doped with a p-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the p-type dopant and host materials used in the p-type doped organic material.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a fluorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula D, but is not limited thereto.

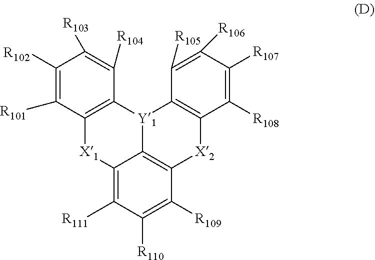

In Formula D,
R₁₀₁ to R₁₁₁ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N—(Ar′₄)(Ar′₅), or may be linked to the adjacent substituents to form a ring(s);
Y′₁ represents B;
X′₁ and X′₂ each independently represent NR′;
R′ represents hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N—(Ar′₄)(Ar′₅); or may be linked to at least one of R₁₀₁, R₁₀₃, R₁₀₉, and R₁₁₁ to form a ring(s);
L′₄ represents a single bond, a substituted or unsubstituted (C₆-C₃₀)arylene, or a substituted or unsubstituted (3- to 30-membered)heteroarylene; and
Ar′₄ and Ar′₅ each independently represent hydrogen, a substituted or unsubstituted (C₁-C₃₀)alkyl, a substituted or unsubstituted (C₂-C₃₀)alkenyl, a substituted or unsubstituted fused ring of (C₃-C₃₀)aliphatic ring and (C₆-C₃₀)aromatic ring, a substituted or unsubstituted (C₆-C₃₀)aryl, or a substituted or unsubstituted (3- to 30-membered)heteroaryl.

Preferably, R₁₀₁ to R₁₁₁ each independently may be hydrogen, deuterium, a substituted or unsubstituted (C₁-C₂₀)alkyl, a substituted or unsubstituted (C₆-C₂₅)aryl, a substituted or unsubstituted (5- to 20-membered)heteroaryl, or -L′₄-N—(Ar′₄)(Ar′₅), or may be linked to the adjacent substituents to form a ring(s).

More preferably, R₁₀₁ to R₁₁₁ each independently may be hydrogen, deuterium, unsubstituted (C₁-C₁₀)alkyl; (C₆-C₁₃)aryl unsubstituted or substituted with at least one of (C₁-C₁₀)alkyl, (13- to 18-membered)heteroaryl, and di(C₆-C₁₃)arylamino; (5- to 18-membered)heteroaryl unsubstituted or substituted with at least one (C₁-C₁₀)alkyl; or -L′₄-N—(Ar′₄)(Ar′₅), or may be linked to the adjacent substituents to form a ring(s). For example, R₁₀₁ to R₁₁₁ each independently may be hydrogen, methyl, tert-butyl, a substituted or unsubstituted phenyl, biphenyl, terphenyl, triphenylenyl, carbazolyl, phenoxazinyl, phenothiazinyl, dimethylacridinyl, dimethylxanthenyl, diphenylamino unsubstituted or substituted with at least one of methyl and diphenylamino, phenylnaphthylamino, dibiphenylamino, phenylamino substituted with phenylcarbazolyl or dibenzofuranyl, (17- to 21-membered)heteroaryl substituted with at least one of methyl and phenyl, or may be linked to the adjacent substituents to form a benzene ring, an indole ring substituted with at least one of phenyl and diphenylamino, a benzofuran ring, a benzothiophene ring, or a 19-membered heterocyclic ring substituted with at least one methyl. The substituent of the substituted phenyl may be at least one of methyl, carbazolyl, dibenzofuranyl, diphenylamino, phenoxazinyl, phenothiazinyl, and dimethylacridinyl.

According to one embodiment, the specific examples of the dopant compound include the following, but are not limited thereto.

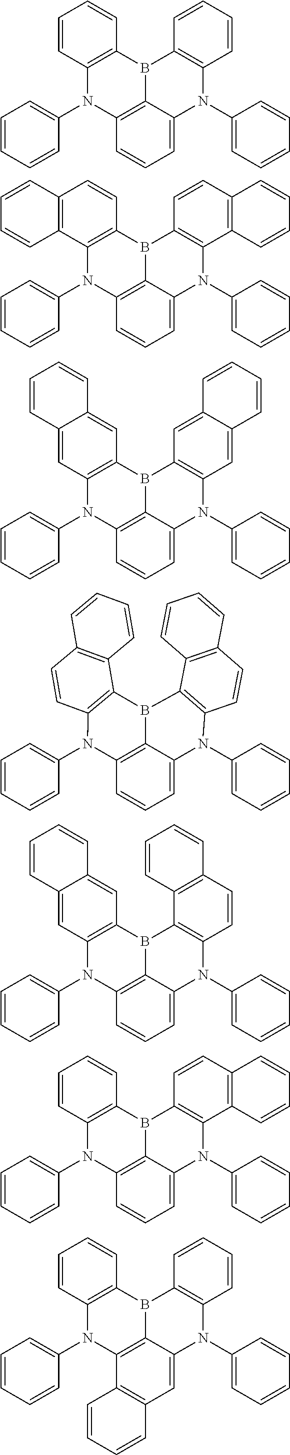

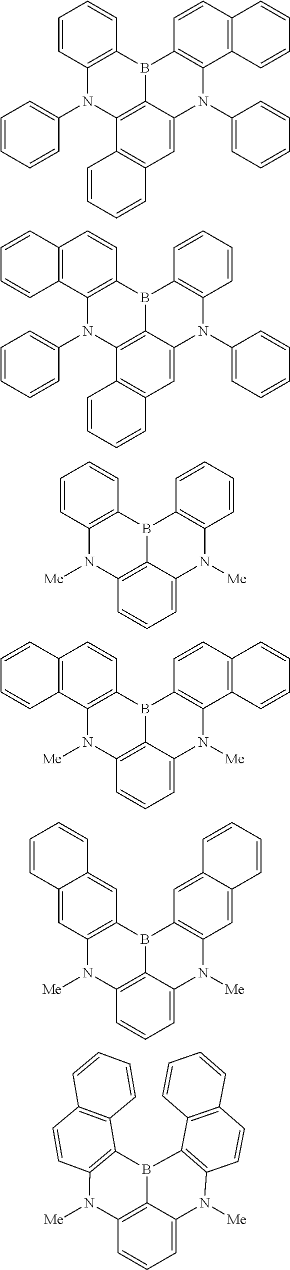

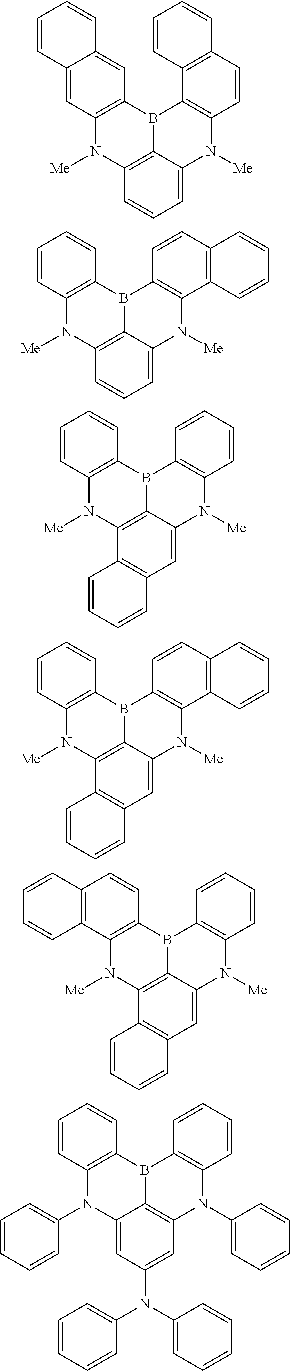

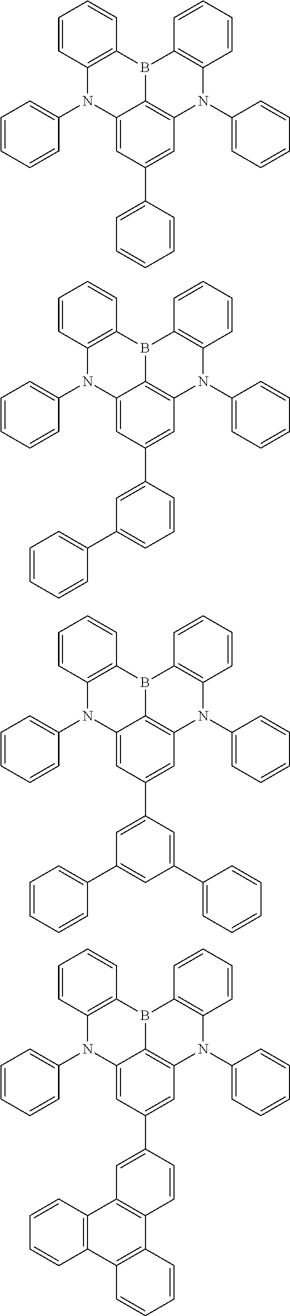

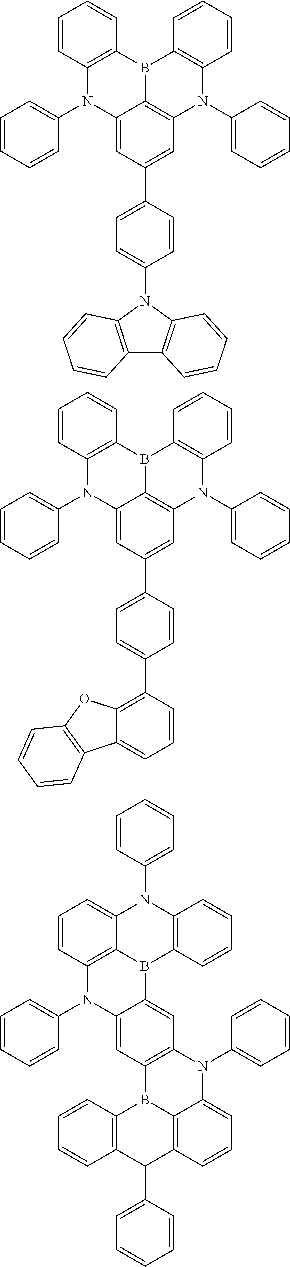

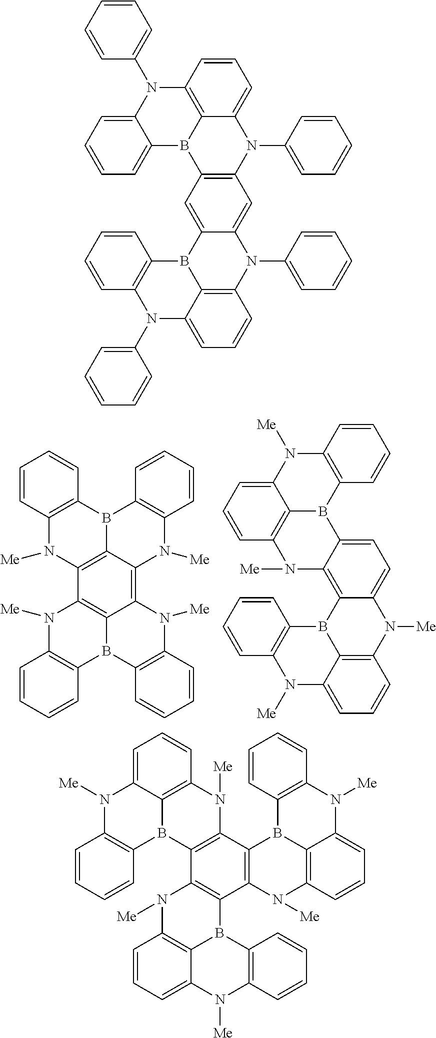

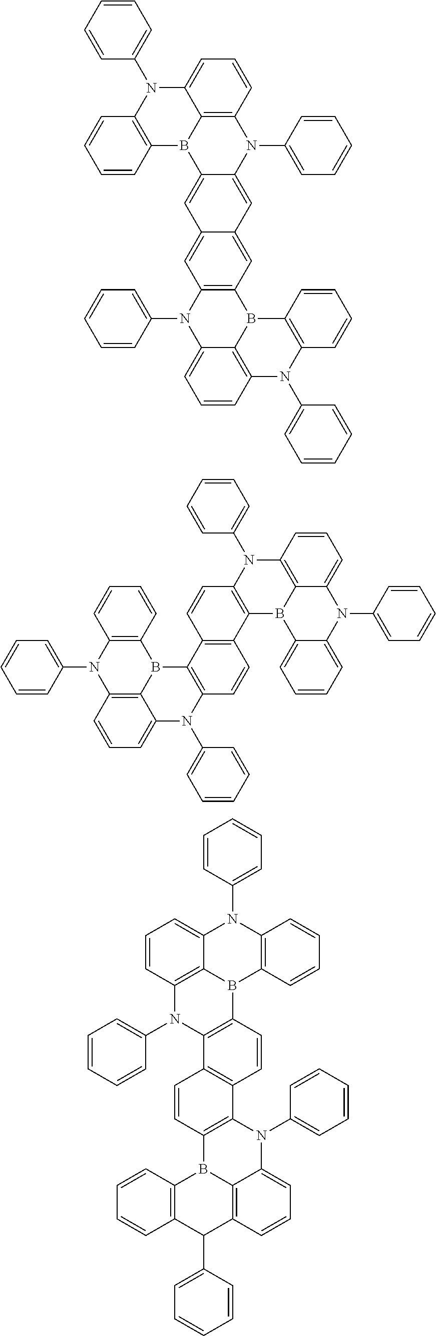

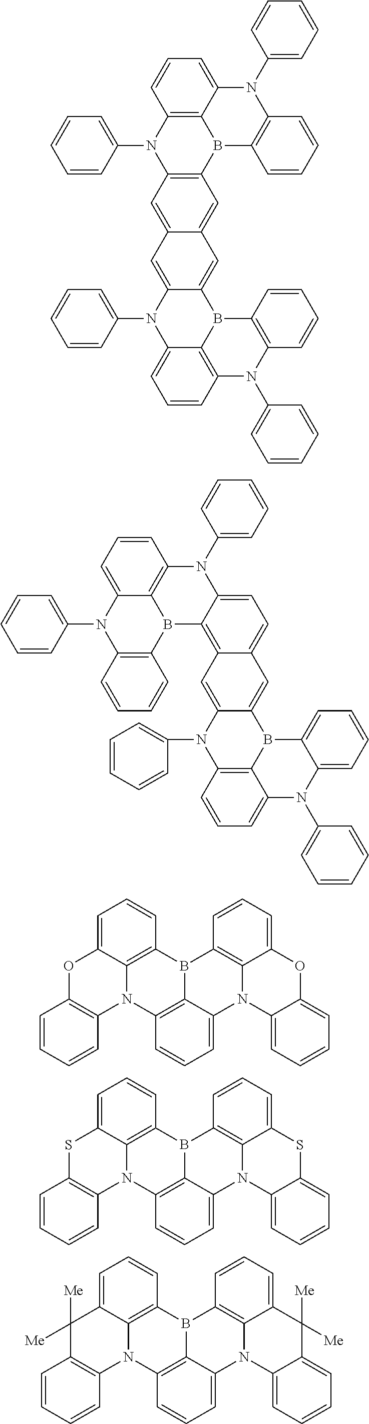

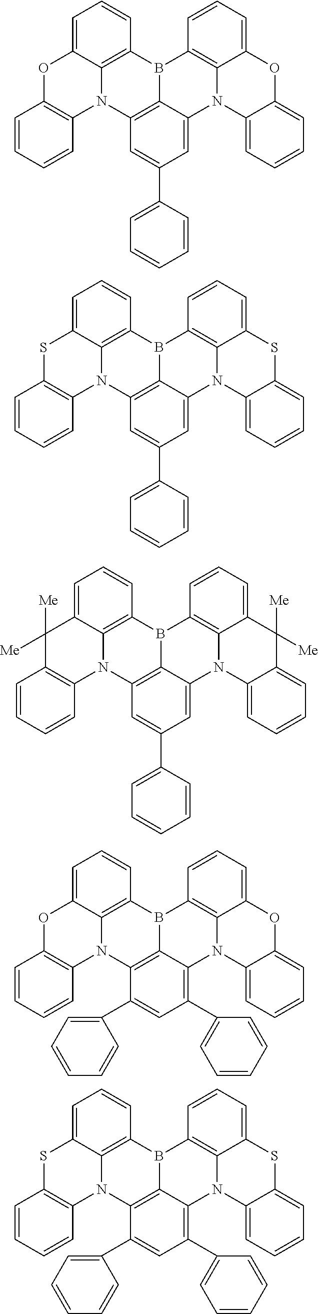

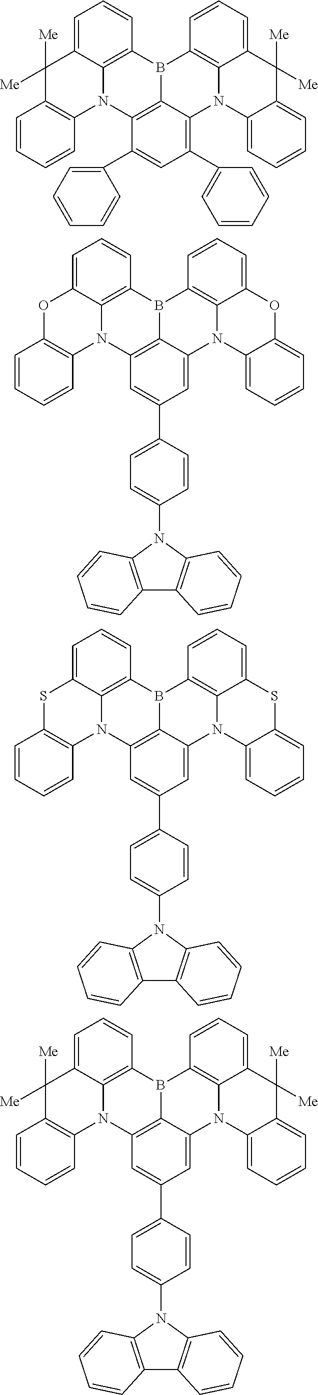

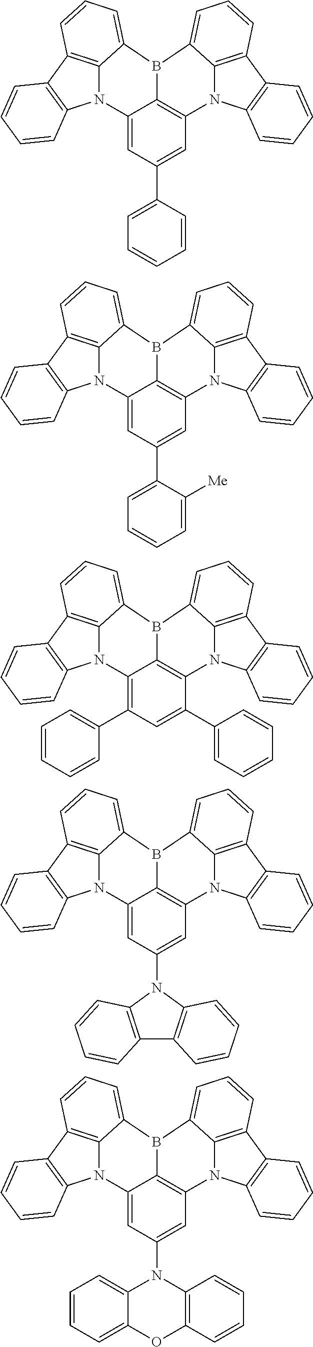

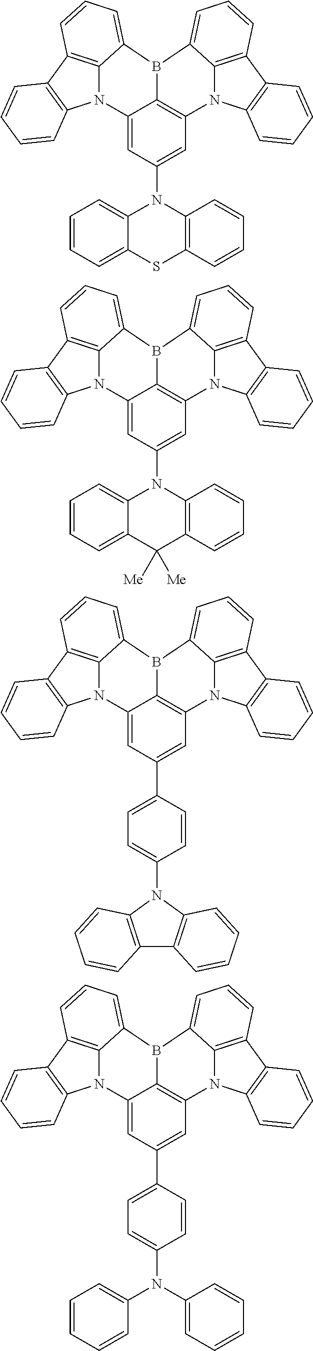

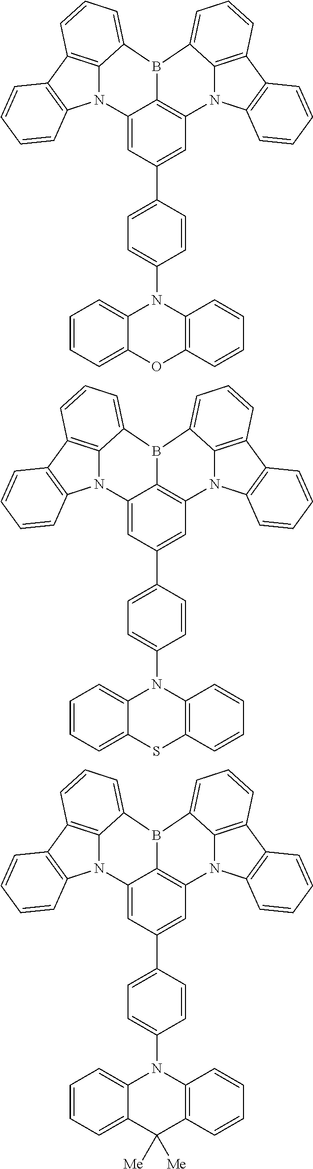

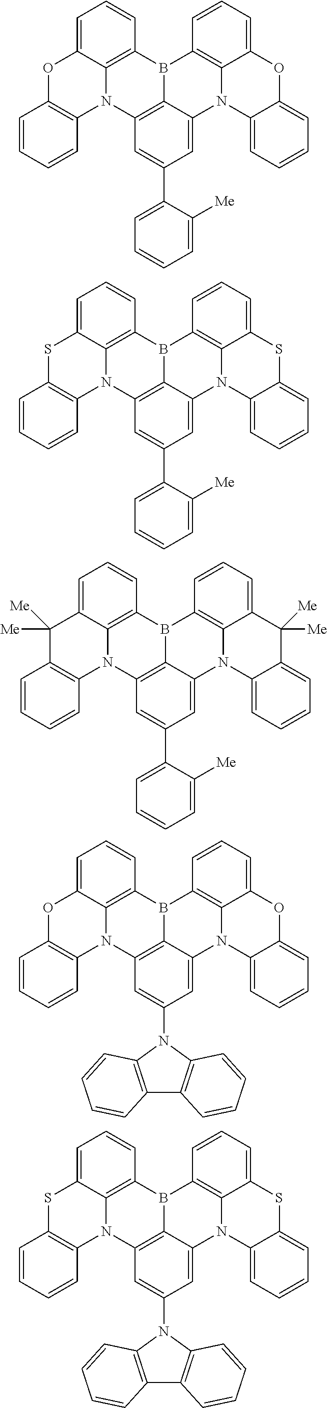

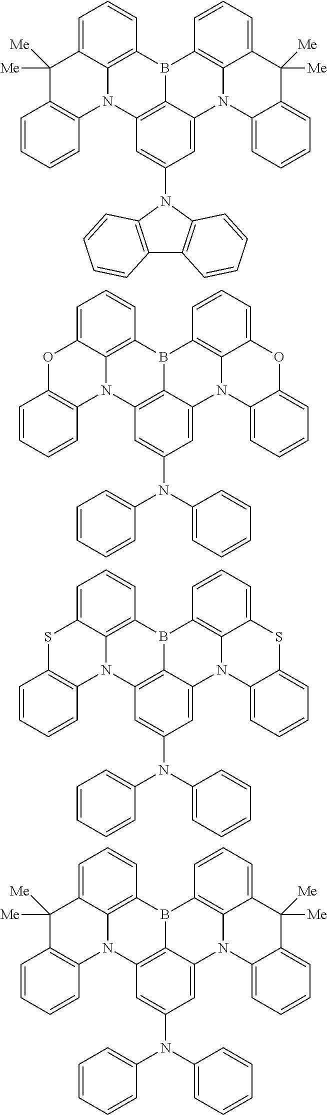

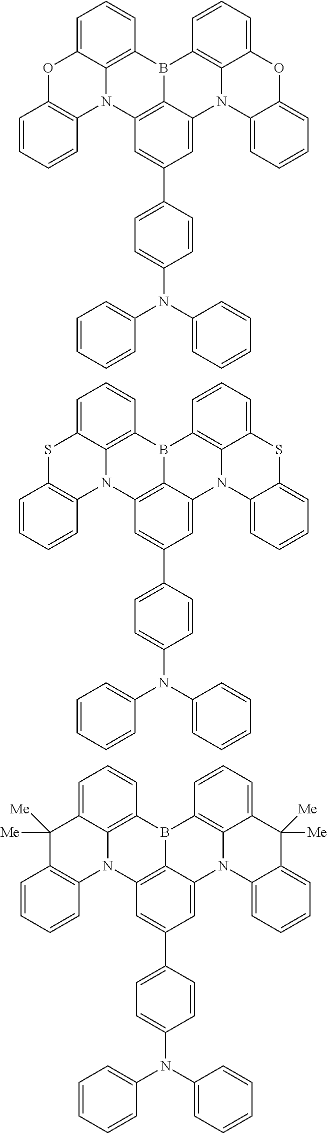

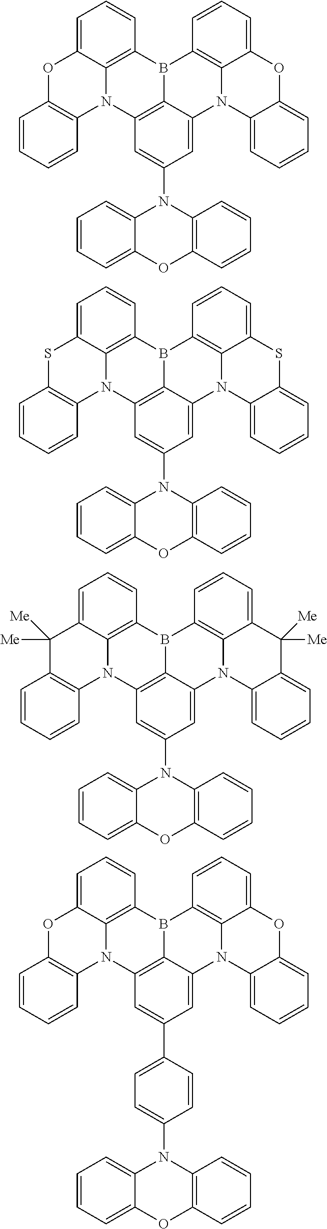

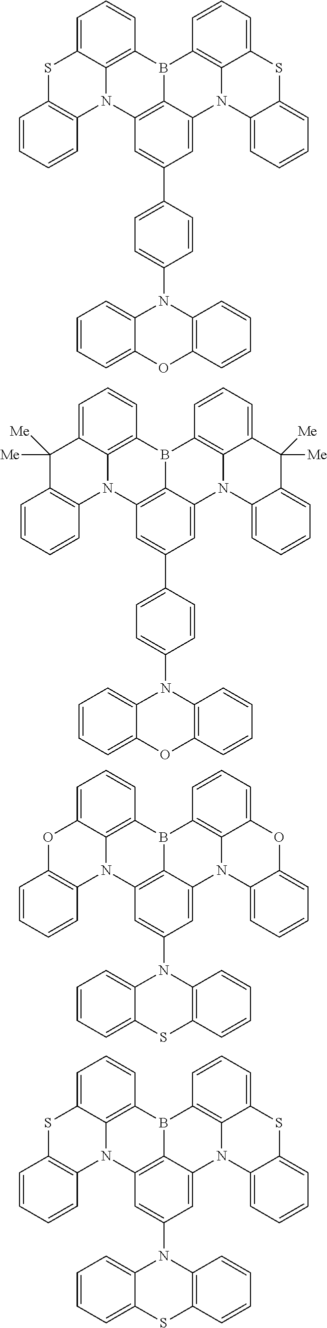

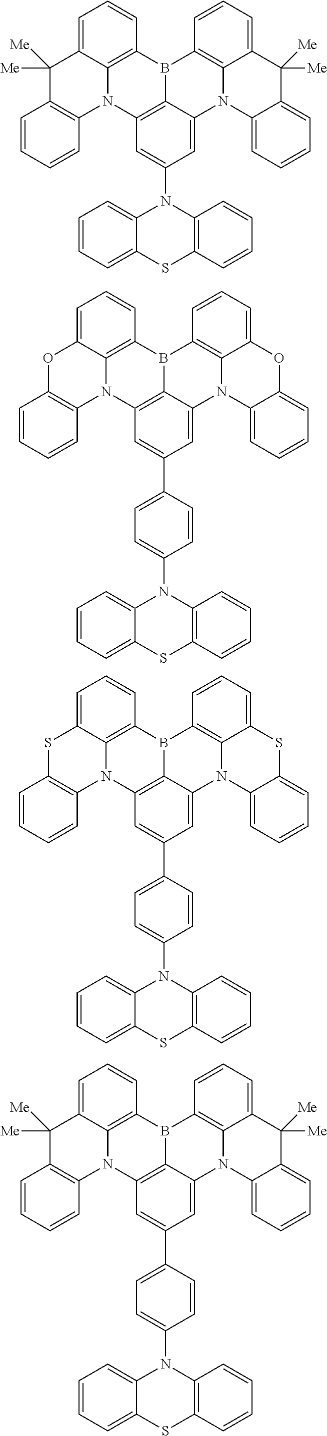

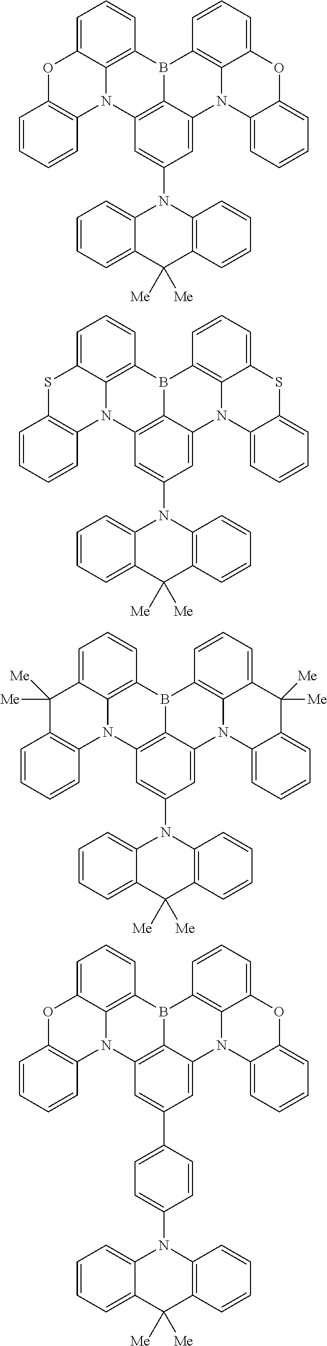

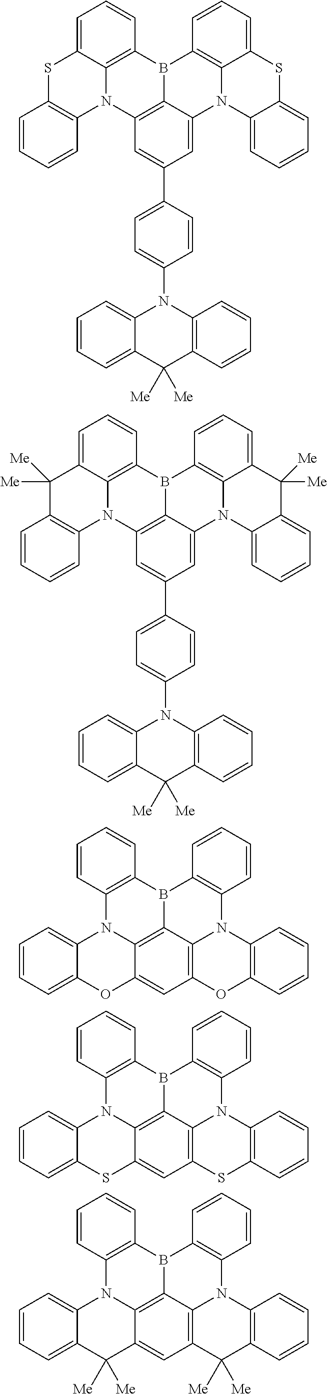

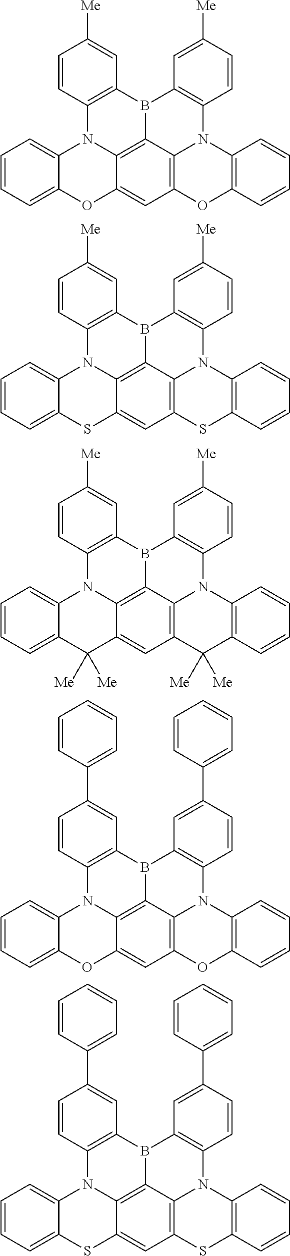

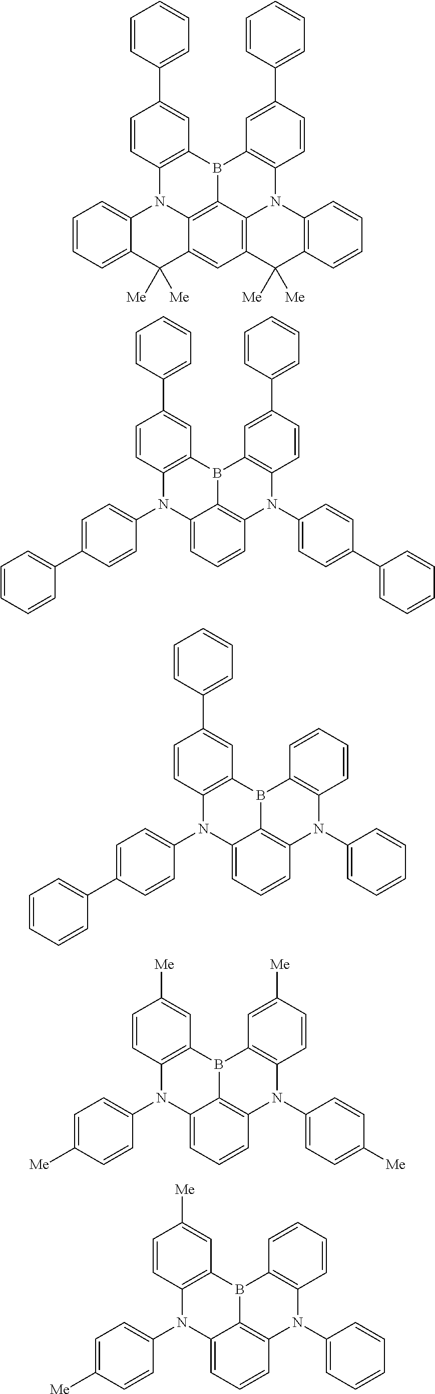

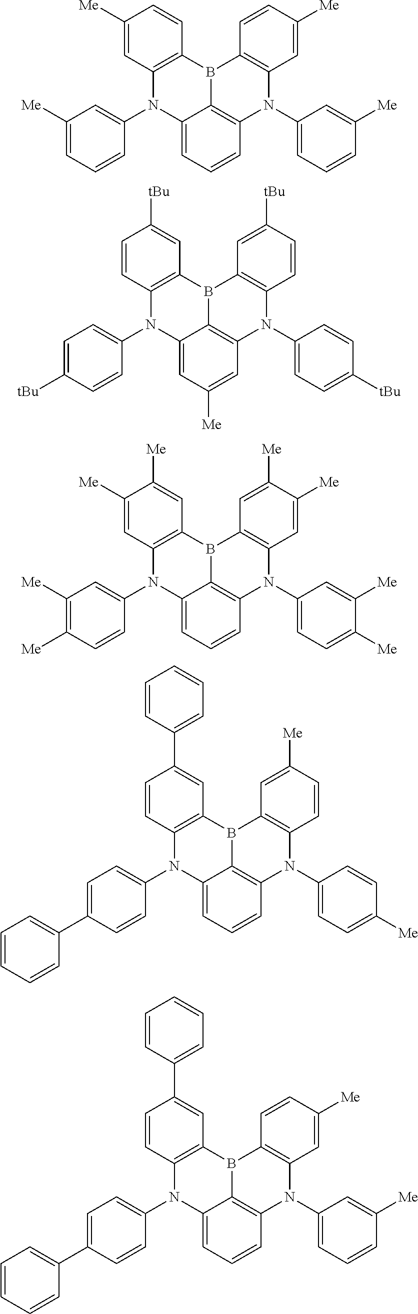

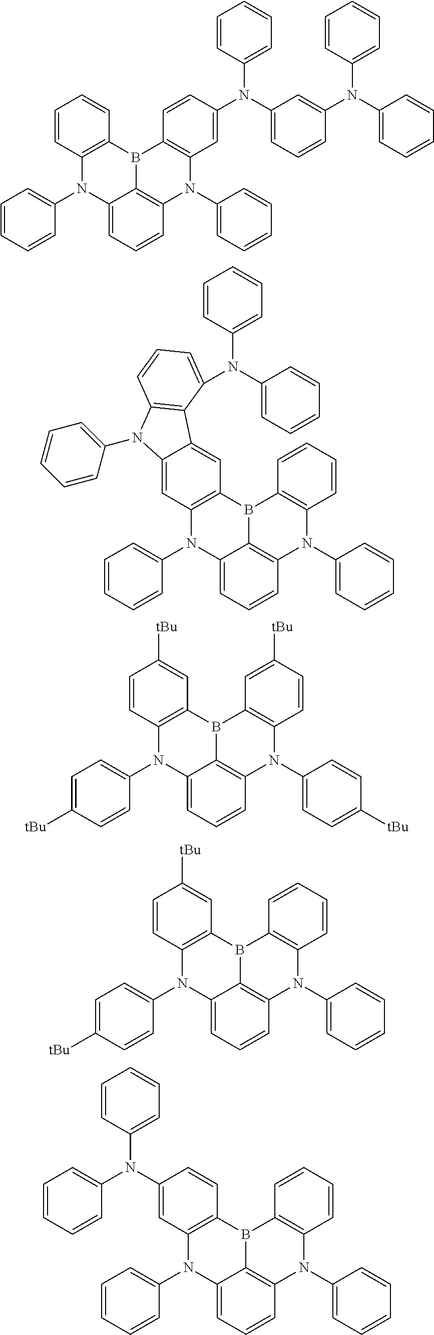

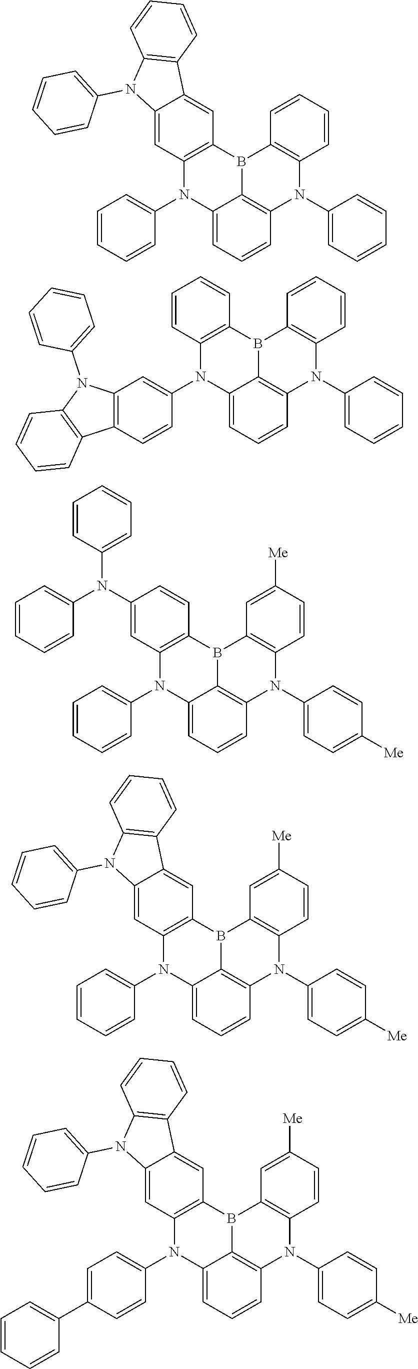

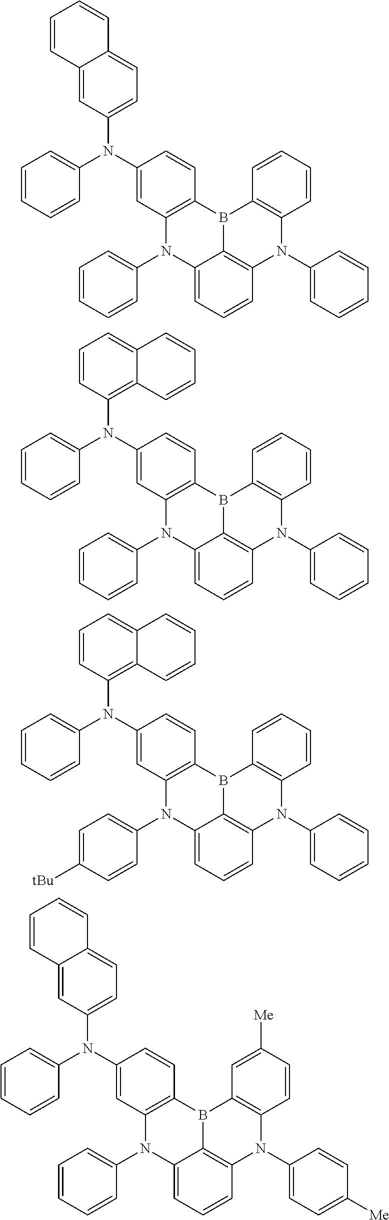

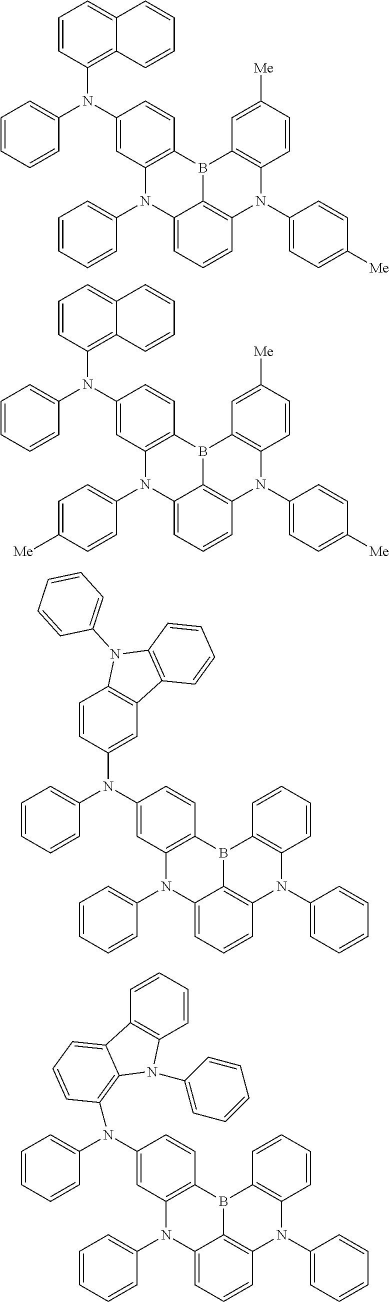

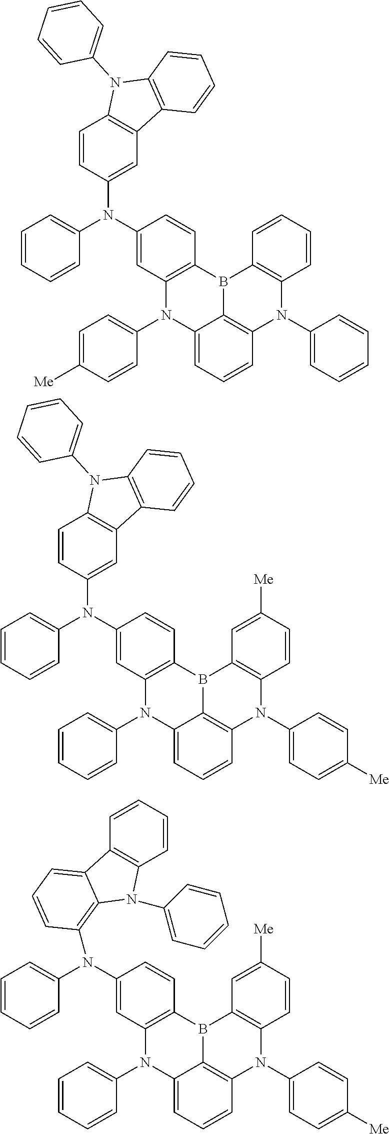

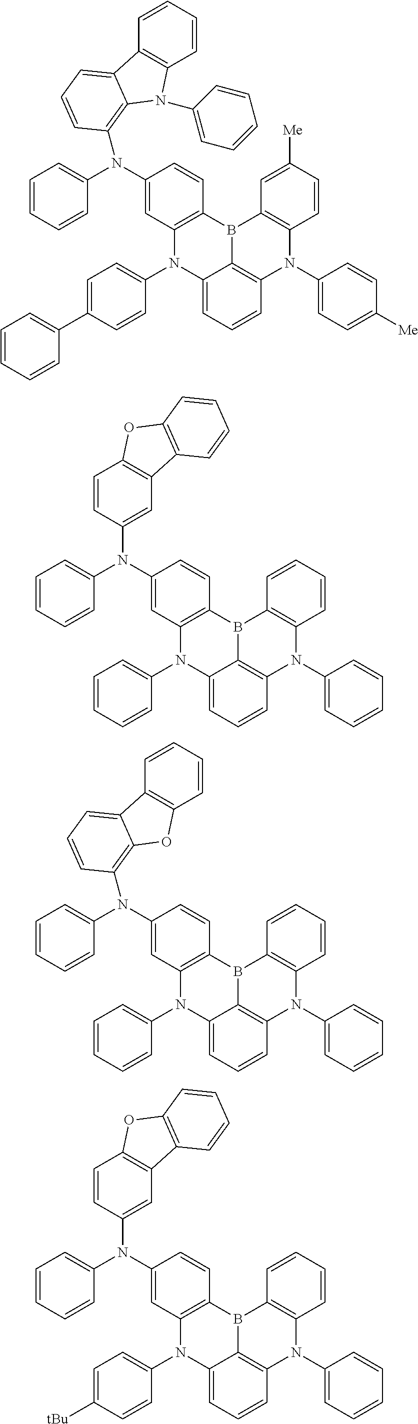

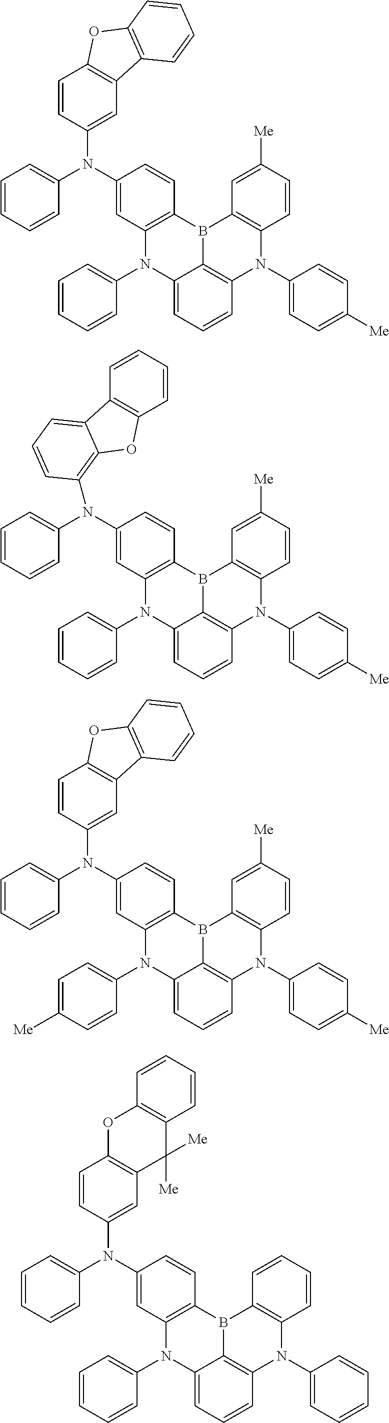

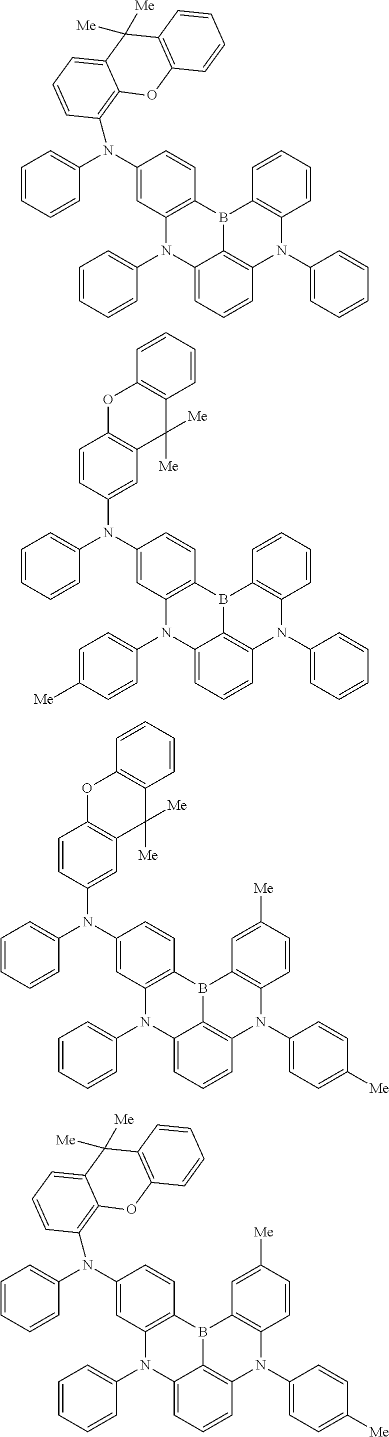

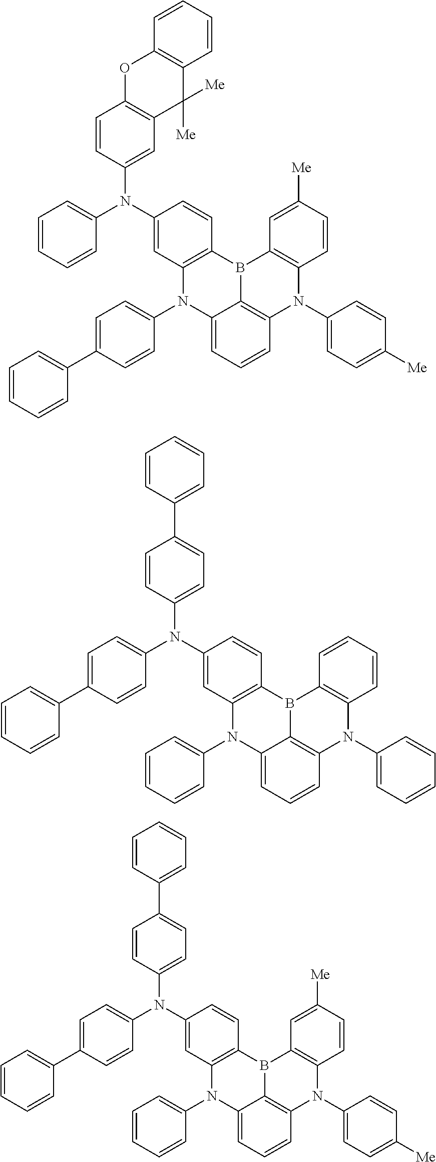

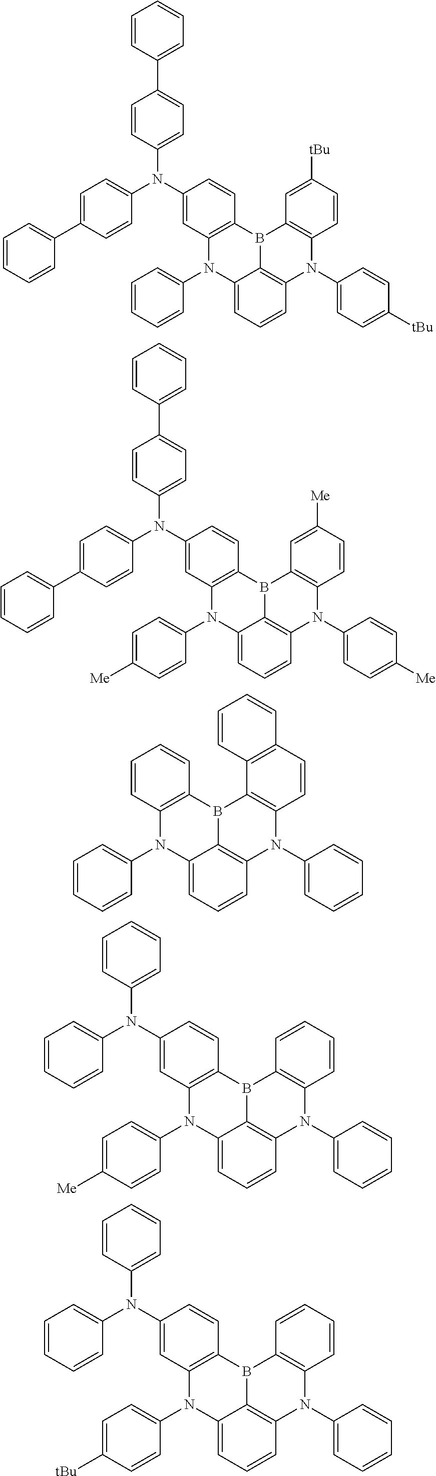

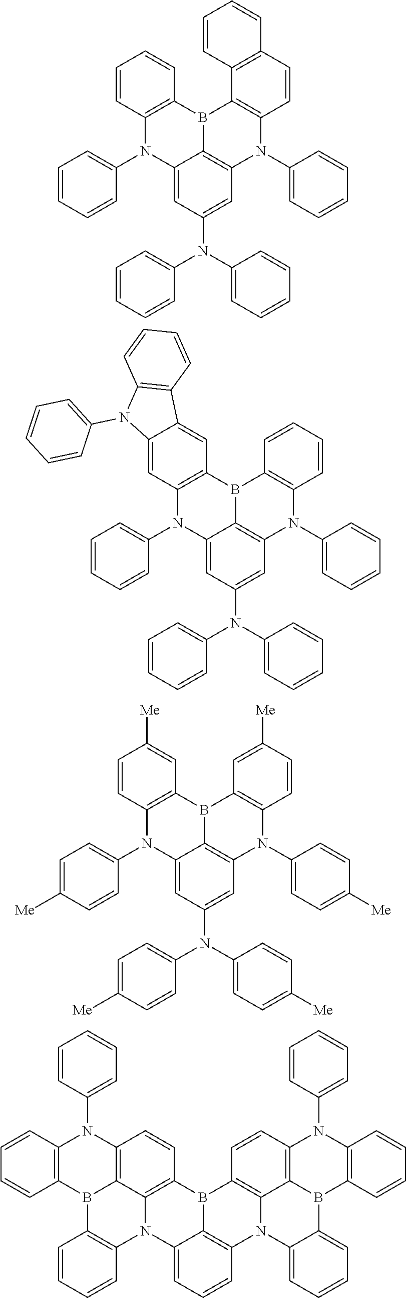

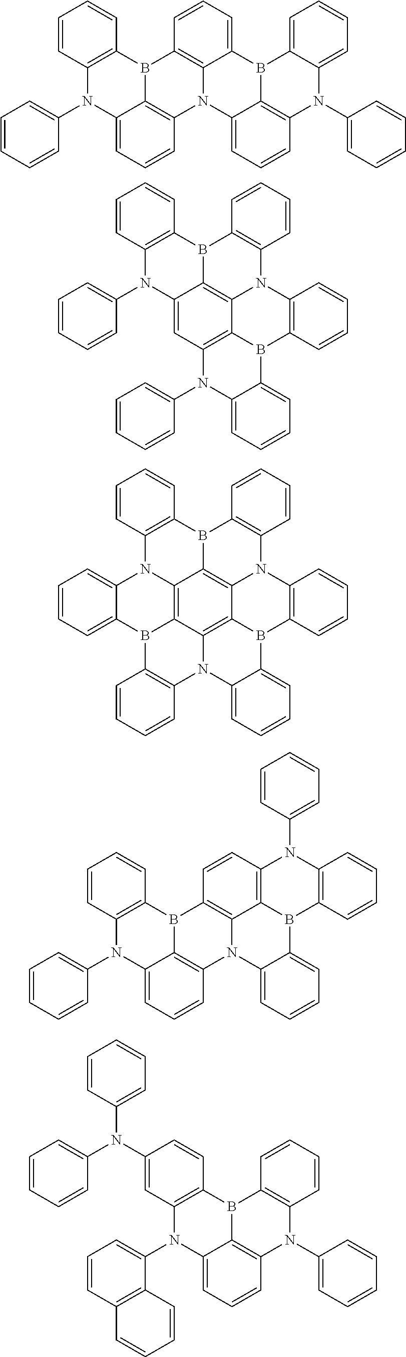

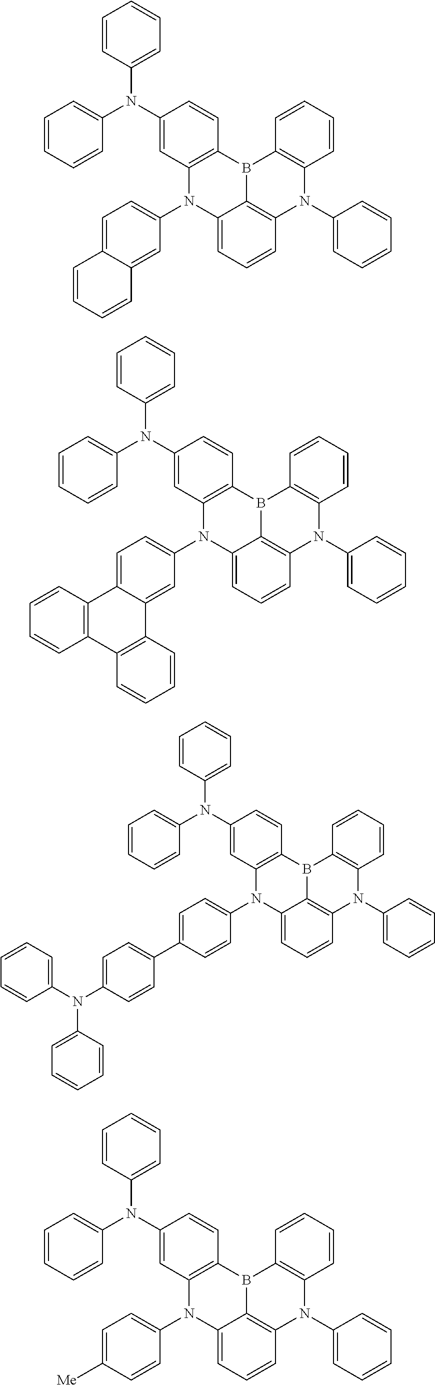

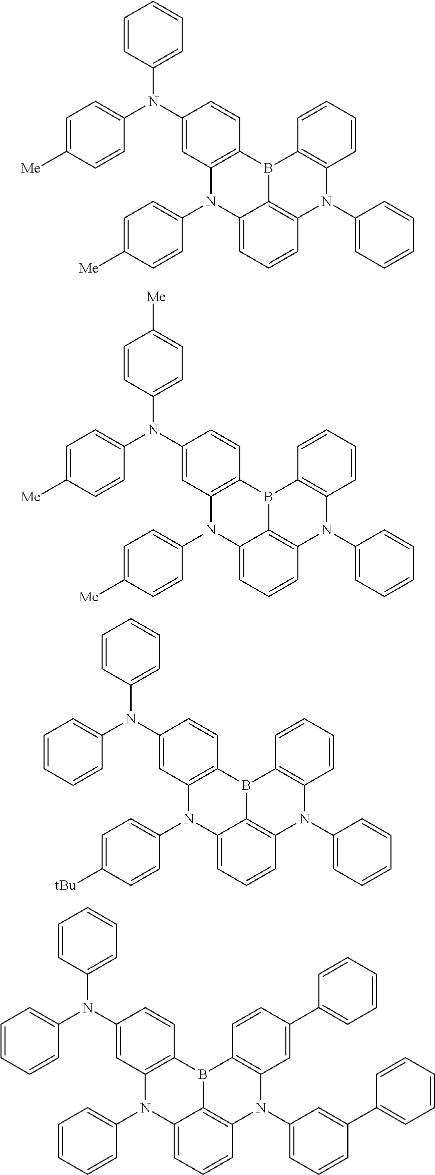

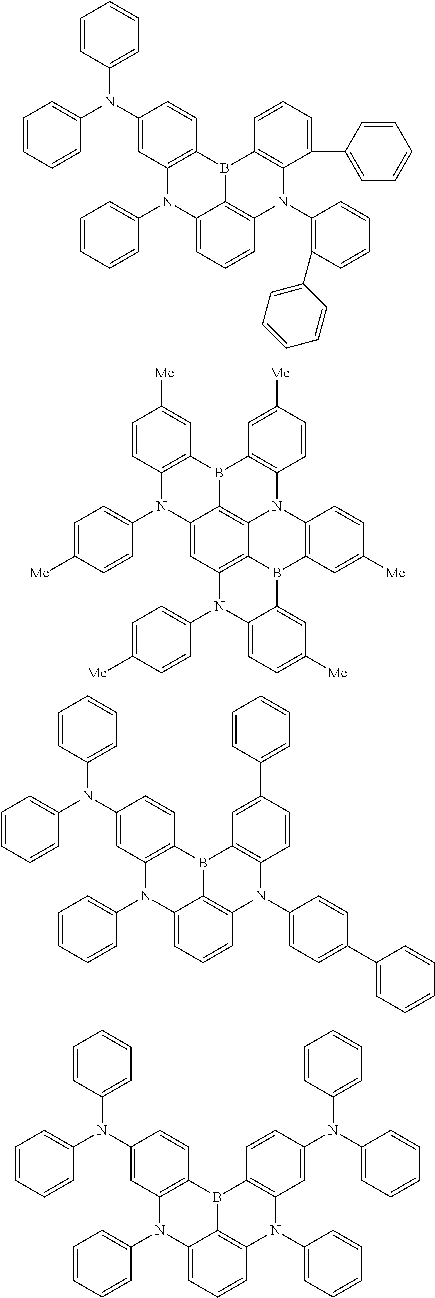

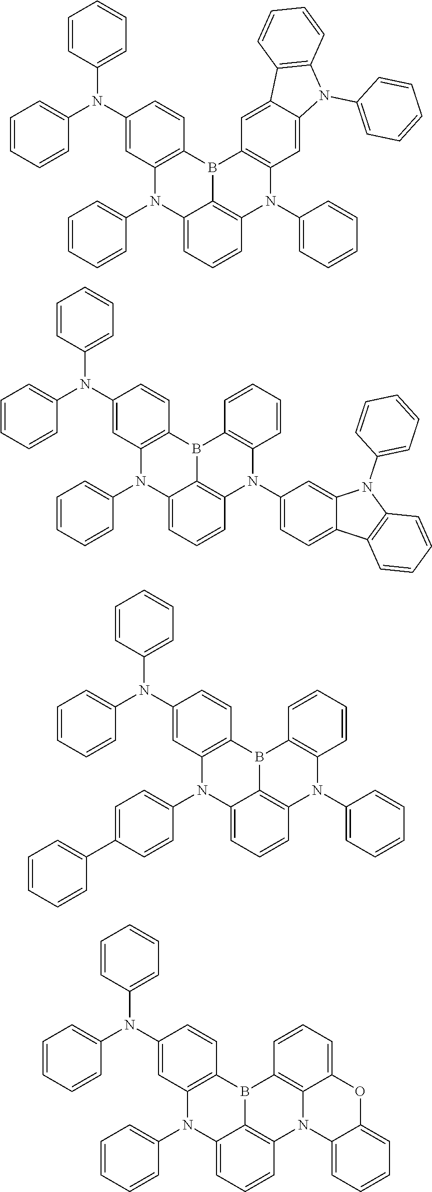

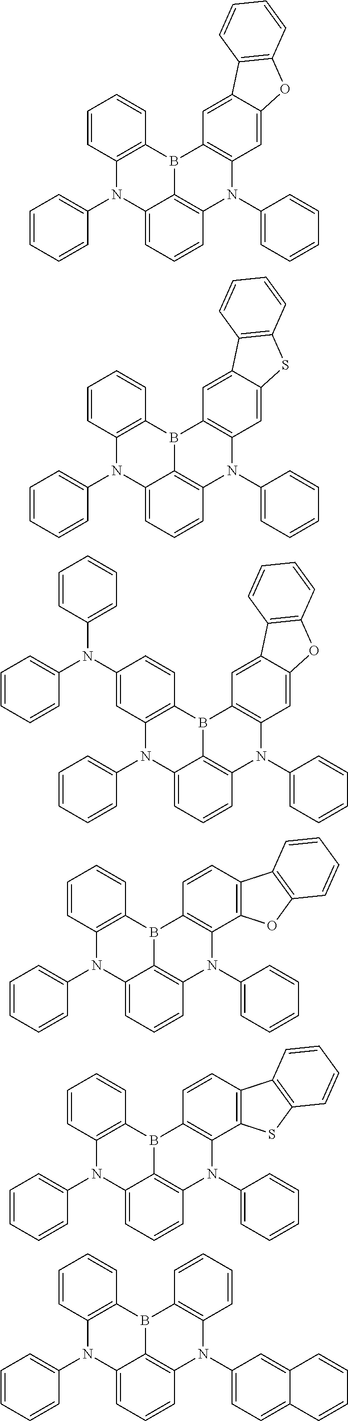

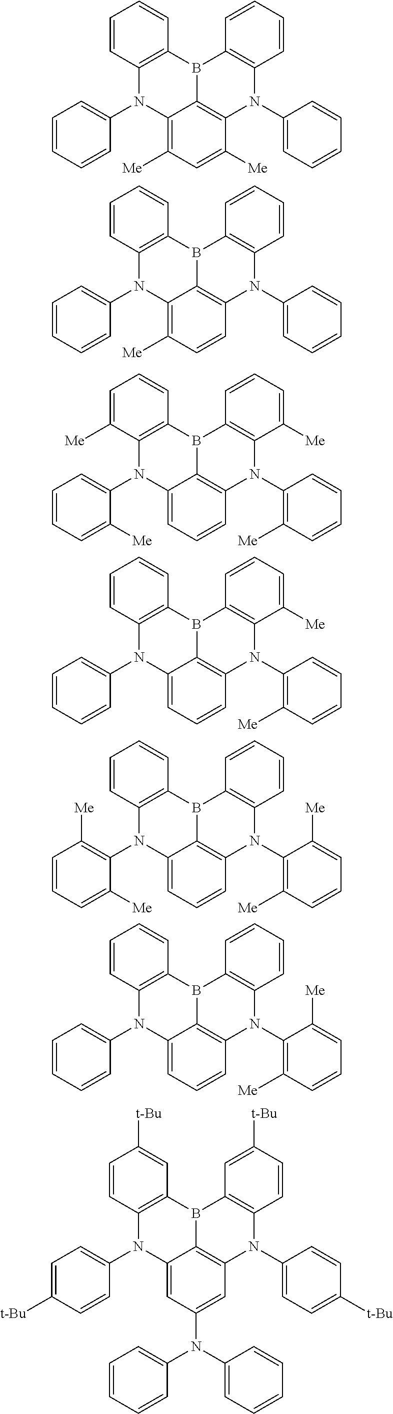

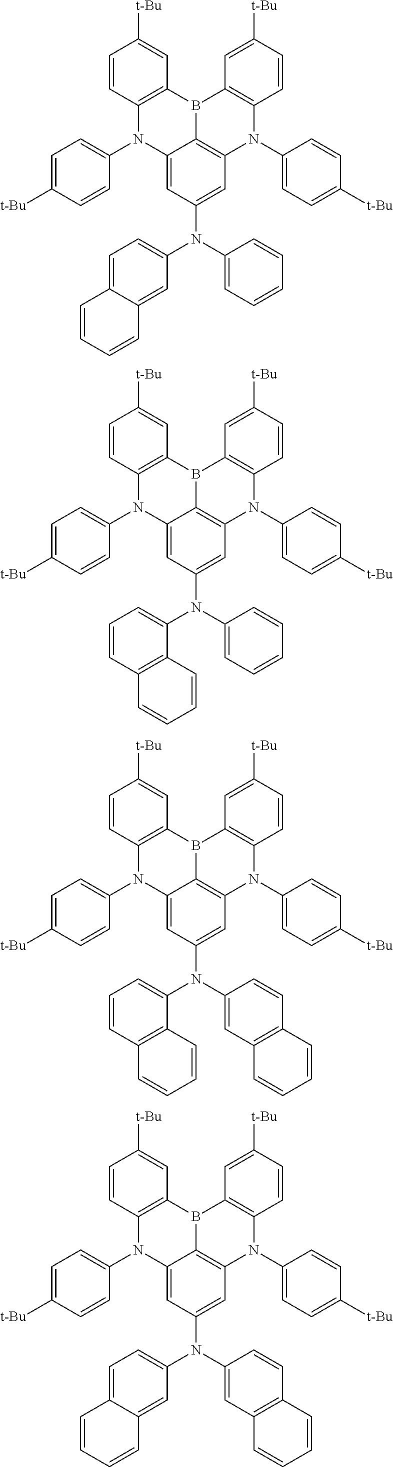

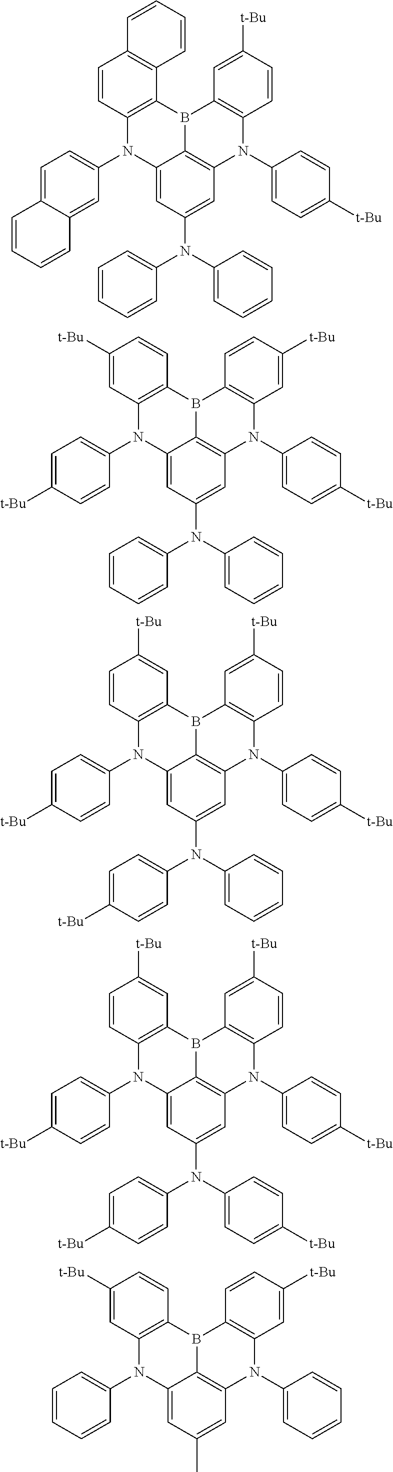

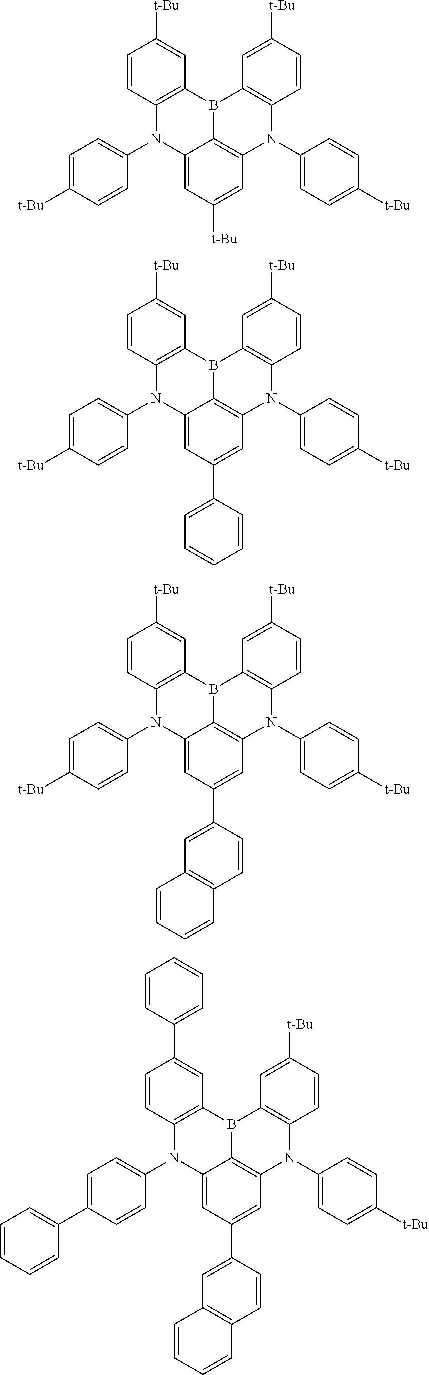

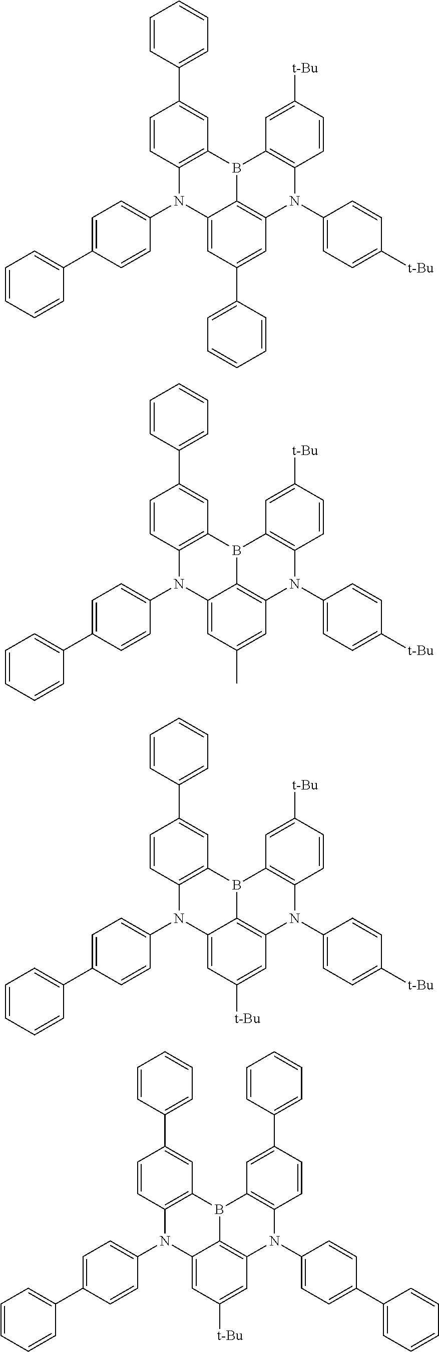

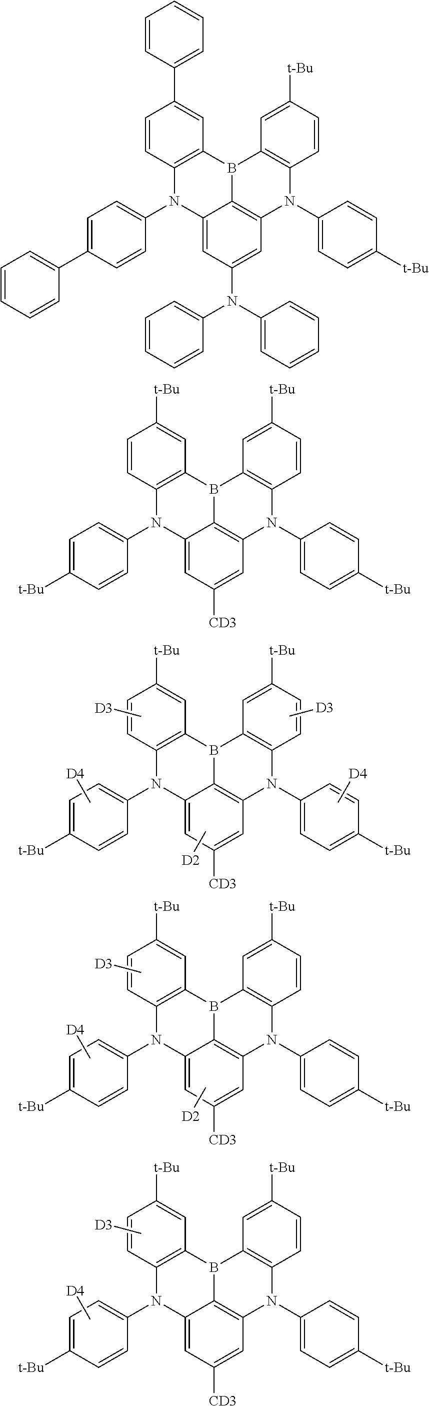

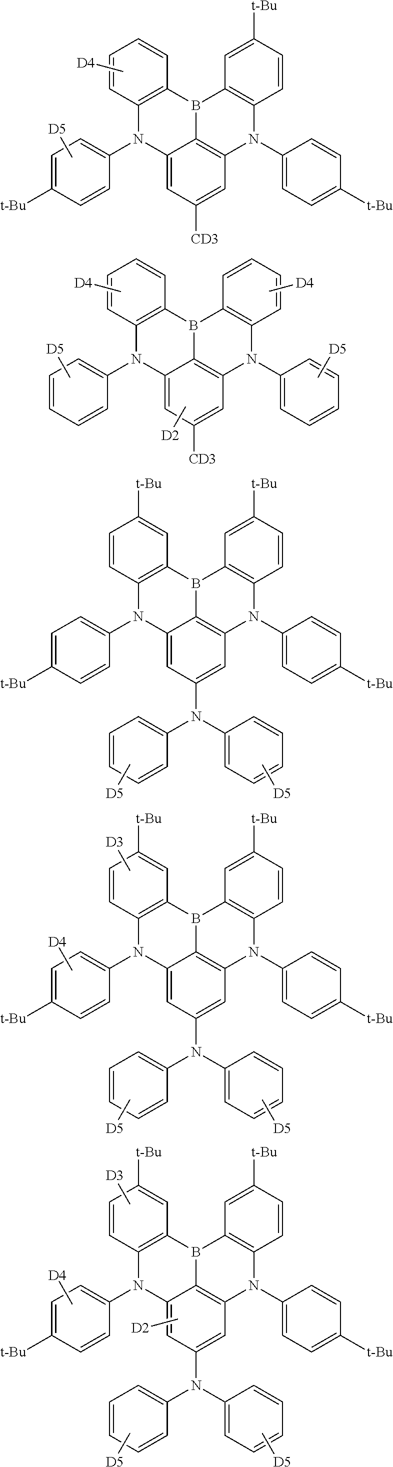

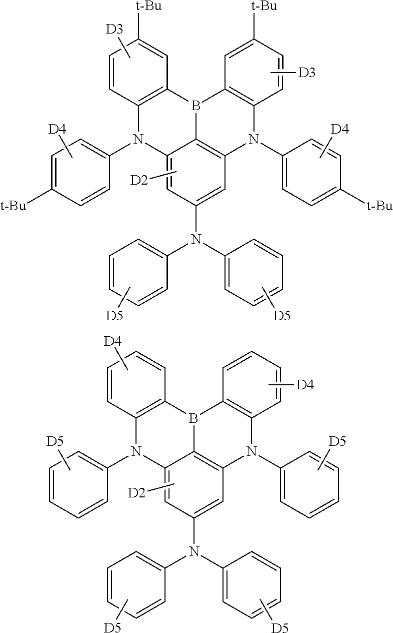

In the above compounds, D2 to D5 each mean that 2 to 5 hydrogens are replaced with deuterium.

In order to form each layer of the organic electroluminescent device of the present disclosure, dry film-forming methods such as vacuum evaporation, sputtering, plasma, ion plating methods, etc., or wet film-forming methods such as spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host compound and the second host compound according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials; and the mixed deposition is a method in which two or more materials are mixed in one crucible source before the deposition, and then a current is applied to one cell to evaporate the materials.

According to one embodiment, when the first host compound and the second host compound are present in the same layer or different layers in the organic electroluminescent device, the two host compounds may be individually formed. For example, after depositing the first host compound, a second host compound may be deposited.

According to one embodiment, the present disclosure can provide display devices comprising a plurality of host materials including a first host compound represented by Formula 1 and a second host compound represented by Formula 2. In addition, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of the host compound according to the present disclosure will be explained with reference to the synthesis method of a representative compound or intermediate compound in order to understand the present disclosure in detail.

### [Example 1] Preparation of Compound H1-31

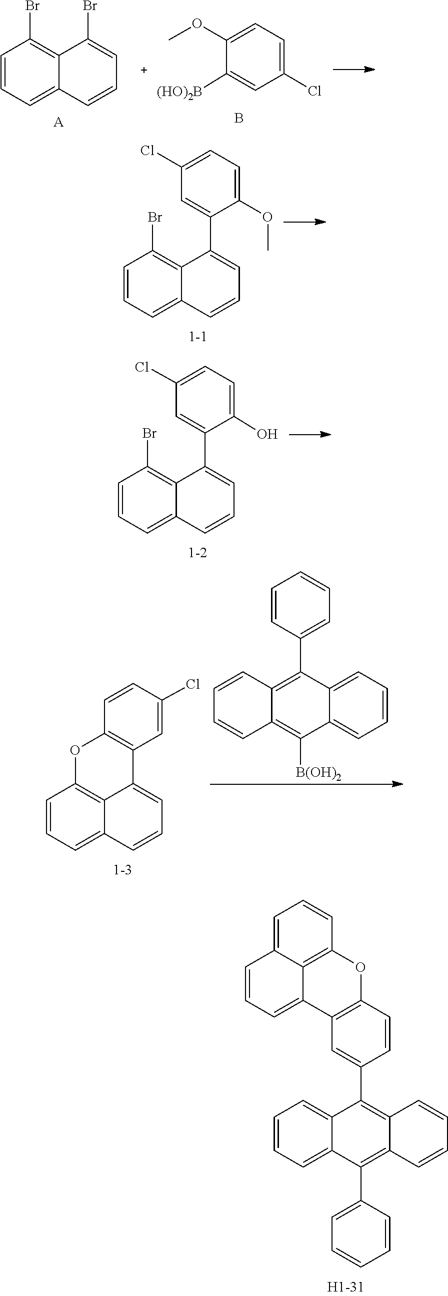

### 1) Synthesis of Compound 1-1

Compound A (23.5 g, 126 mmol), Compound B (30 g, 105 mmol), tetrakis(triphenylphosphine)palladium(0) (Pd(PPh₃)₄) (6 g, 5.25 mmol), 131 mL of 2 M K₂CO₃, 524 mL of toluene, and 131 mL of ethanol (EtOH) were added to a flask and dissolved, and then reacted at 80° C. for 1 hour. After completion of the reaction, the organic layer was extracted with ethyl acetate, the residual moisture was removed using magnesium sulfate, followed by drying, and column chromatography separation was then performed to obtain Compound 1-1 (31 g, yield: 85%).

### 2) Synthesis of Compound 1-2

Compound 1-1 (30.5 g, 87.7 mmol) was dissolved in 88 mL of dichloromethane (DCM), the reactor temperature was lowered to 0° C., and 132 mL of 1 M boron tribromide (BBr₃) was slowly added dropwise. After 30 minutes, the reactor was brought to room temperature and stirred for 16 hours. The mixture was neutralized by adding an aqueous solution of potassium carbonate to the reactor, the organic layer was extracted with an aqueous solution of sodium thiosulfate, the residual moisture was removed using magnesium sulfate, followed by drying, and column chromatography separation was then performed to obtain Compound 1-2 (24.7 g, yield: 84%).

### 3) Synthesis of Compound 1-3

Compound 1-2 (23.7 g, 71 mmol), CuI (13.5 g, 71 mmol), Cs₂CO₃ (69 g, 213 mmol), and 355 mL of o-xylene were added to a flask and dissolved, and then reacted at 160° C. for 5 hours. After completion of the reaction, the organic layer was extracted with ethyl acetate, the residual moisture was removed using magnesium sulfate, followed by drying, and column chromatography separation was then performed to obtain Compound 1-3 (12.11 g, yield: 67%).

### 4) Synthesis of Compound H1-31

Compound 1-3 (6 g, 24 mmol), (10-phenylanthracene-9-yl)boronic acid (8.5 g, 28 mmol), Pd(OAc)₂ (377 mg, 7 mmol), Ligand (tricyclohexylphosphonium tetrafluoroborate) (1.5 g, 0.36 mmol), K₃PO₄ (15 g, 72 mmol), toluene (480 mL), isopropyl alcohol (120 mL), and distilled water (120 mL) were added to a flask and dissolved, and then stirred under reflux at 120° C. for 3 hours. Next, the reaction mixture was cooled to room temperature, the organic layer was extracted with ethyl acetate, and the residual moisture was removed using magnesium sulfate, followed by drying. Thereafter, distillation was performed under reduced pressure, and column chromatography separation was then performed to obtain Compound H1-31 (4.7 g, yield: 42%).

| | MW | M.P. |
|---|---|---|
| H1-31 | 470.17 | 271° C. |

### [Example 2] Preparation of Compound H1-186

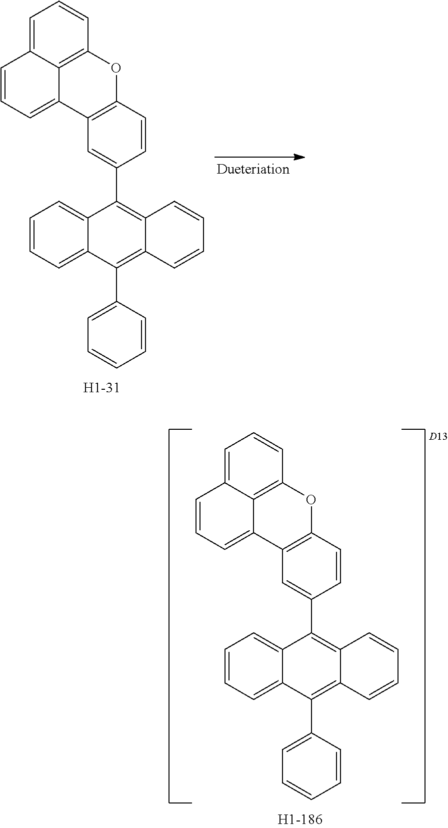

Compound H1-31 was synthesized by selecting the deuteration methods disclosed in Korean Patent Nos. 10-2283849 and 10-1427457, etc. to obtain Compound H1-186 (8.0 g, yield: 72%, MS: [M+H]⁺=483.17).

| | MW | M.P. |
|---|---|---|
| H1-186 | 483.17 | 272° C. |

Hereinafter, the preparation method of an organic electroluminescent device comprising the plurality of host materials and/or the organic electroluminescent compound according to the present disclosure and the device properties thereof will be explained in order to understand the present disclosure in detail.

### [Device Examples 1 to 5] Preparation of OLEDs Comprising a Plurality of Host Materials According to the Present Disclosure

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates, and Compound HI-1 was deposited in a doping amount of 5 wt % based on the total amount of compounds HI-1 and HT-1 to form a hole injection layer having a thickness of 10 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 15 nm on the first hole transport layer. After forming the hole injection layer and the hole transport layers, a light-emitting layer was formed thereon as follows: each of the first host compound and the second host compound described in Table 1 below were introduced at a ratio of 1:1 into two cells of the vacuum vapor deposition apparatus as hosts, respectively, and Compound BD was introduced into another cell as a dopant. The two materials were evaporated at different rates, and were deposited in a doping amount of 2 wt % based on the total amount of the hosts and dopant to form a light-emitting layer having a thickness of 22.5 nm on the second hole transport layer. Next, Compound ET-1 was deposited on the light-emitting layer to form an electron buffer layer with a thickness of 5 nm. Next, Compound EI-1 and Compound EI-2 were added to two other cells and evaporated at a ratio of 2:1 to form an electron transport layer having a thickness of 25 nm on the electron buffer layer. Next, Compound Yb:LiF was added to two another cells as an electron injection layer, evaporated at a ratio of 2:1 to form an electron injection layer having a thickness of 1 nm on the electron transport layer. Next, an Al cathode having a thickness of 80 nm was deposited on the electron injection layer by another vacuum vapor deposition apparatus. Thus, OLEDs were produced. Each compound used for all the materials were purified by vacuum sublimation at 10⁻⁶ Torr.

### [Device Comparative Example 1] Preparation of an OLED Comprising a Single Host Material

An OLED was manufactured in the same manner as in Device Example 1, except that Compound H1-31 was used alone as the host material of the light-emitting layer.

The driving voltage at a luminance of 1,000 nits and the minimum time taken for luminance to decrease from 100% to 95% at a luminance according to conversion efficiency standards (lifespan: T₉₅) of the OLEDs of Device Examples 1 to 5 and Device Comparative Example 1 produced as described above were measured, and the results thereof are shown in Table 1 below.

TABLE 1

| | | | Driving | Conversion | |
|---|---|---|---|---|---|
| | First Host | Second Host | Voltage | Efficiency | Lifespan |
| | Compound | Compound | (V) | (eff/Y) | (T₉₅, hr) |
| Device Example 1 | 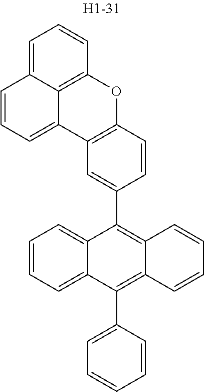 [SMILES: c1ccc(-c2c3ccccc3c(-c3ccc4c(c3)-c3cccc5cccc(c35)O4)c3ccccc23)cc1] | 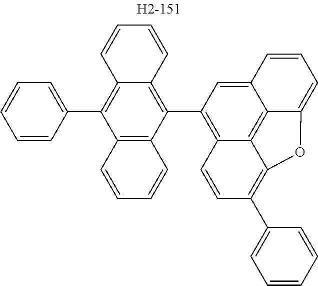 [SMILES: c1ccc(-c2c3ccccc3c(-c3cc4cccc5oc6c(-c7ccccc7)ccc3c6c45)c3ccccc23)cc1] | 3.6 | 89.6 | 68 |
| Device Example 2 | 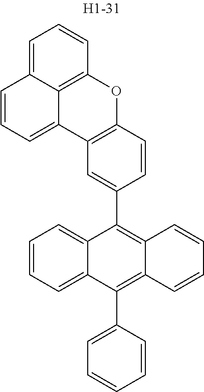 [SMILES: c1ccc(-c2c3ccccc3c(-c3ccc4c(c3)-c3cccc5cccc(c35)O4)c3ccccc23)cc1] | 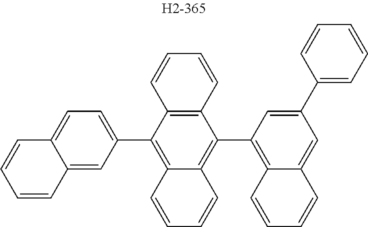 [SMILES: c1ccc(-c2cc(-c3c4ccccc4c(-c4ccc5ccccc5c4)c4ccccc34)c3ccccc3c2)cc1] | 3.7 | 87.9 | 86 |
| Device Example 3 | 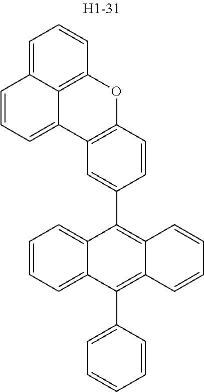 [SMILES: c1ccc(-c2c3ccccc3c(-c3ccc4c(c3)-c3cccc5cccc(c35)O4)c3ccccc23)cc1] | 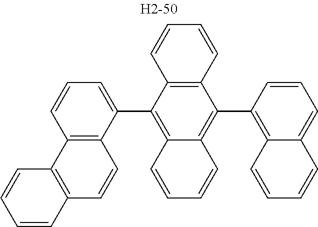 [SMILES: c1ccc2c(-c3c4ccccc4c(-c4cccc5c4ccc4ccccc45)c4ccccc34)cccc2c1] | 3.7 | 89.4 | 86 |
| Device Example 4 | 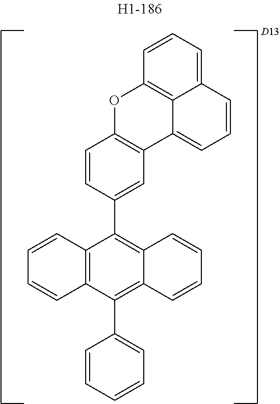 | 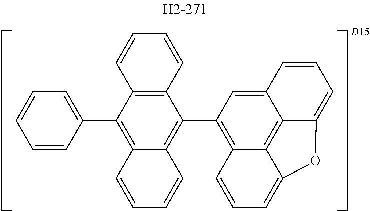 | 3.5 | 84.4 | 201 |
| Example 5 | 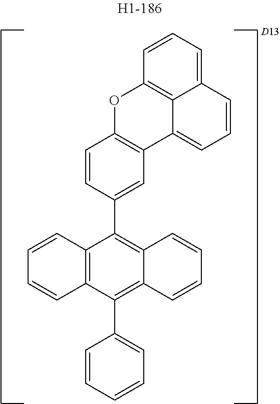 | 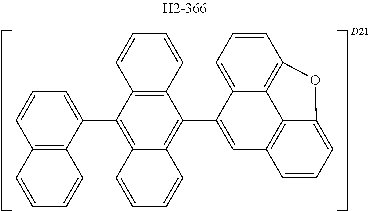 | 3.5 | 90.6 | 176 |
| Device Compar- ative Example 1 | 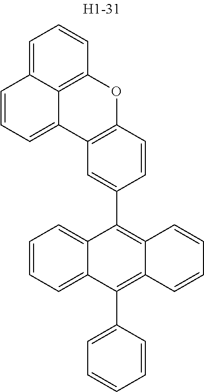 [SMILES: c1ccc(-c2c3ccccc3c(-c3ccc4c(c3)-c3cccc5cccc(c35)O4)c3ccccc23)cc1] | — | 3.6 | 76.4 | 64 |

As shown in Table 1 above, it can be confirmed that the organic electroluminescent device comprising a plurality of host materials according to the present disclosure exhibits lower voltage and/or higher luminous efficiency and/or longer lifespan characteristics than the organic electroluminescent device containing only a single host material.

### [Device Example 6] Preparation of an OLED Comprising a Single Host Material

An OLED was manufactured in the same manner as in Device Example 1, except that Compound HI-186 described in Table 2 below was used alone as the host material of the light-emitting layer.

The driving voltage at a luminance of 1,000 nits and the minimum time taken for luminance to decrease from 100% to 95% at a luminance according to conversion efficiency standards (lifespan: T₉₅) of the OLED of Device Example 6 produced as described above were measured, and the results thereof are shown in Table 2 below.

TABLE 2

| | Host | Driving | Lifespan |
|---|---|---|---|
| | Compound | Voltage (V) | (T₉₅, hr) |
| Device | H1-186 | 3.6 | 172 |
| Example 6 | | | |

The compounds used in Device Examples 1 to 6 and Device Comparative Example 1 are specifically shown in Table 3 below.

TABLE 3

| | | | |
|---|---|---|---|
| Hole Injection Layer/Hole Transport Layer | 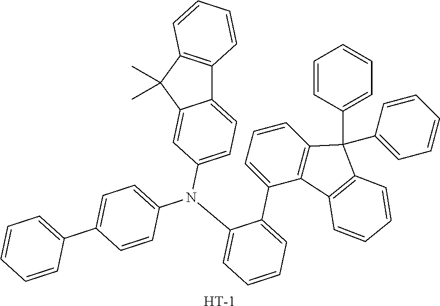 | HI-1 | HT-2 |
| Light-Emitting Layer | | | |
| Electron Buffer Layer | | | |
| Electron Transport Layer/Electron Injection Layer | | | |

1. A plurality of host materials comprising at least one first host compound and at least one second host compound, wherein the first host compound is represented by the following Formula 1, the second host compound is represented by the following Formula 2, and the first host compound and the second host compound are different from each other: 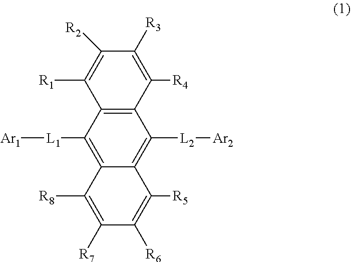
wherein,
R₁ to R₈ each independently are selected from hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di-(C₁-C₃₀)alkylamino, a substituted or unsubstituted mono- or di-(C₂-C₃₀)alkenylamino, a substituted or unsubstituted mono- or di-(C₆-C₃₀)arylamino, a substituted or unsubstituted mono- or di-(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, and a combination thereof;
L₁ and L₂ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl, or a substituted or unsubstituted (3- to 30-membered)heteroaryl; and
Ar₂ is represented by the following Formula 1-A: 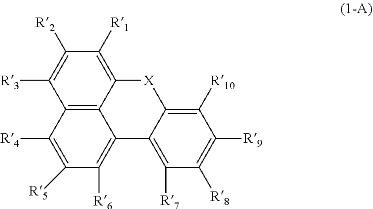
wherein,
X represents —O— or —S—;
R′₁ to R′₁₀ each independently represent hydrogen, deuterium, a substituted or unsubstituted (C₆-C₃₀)aryl, or a substituted or unsubstituted (3- to 30-membered)heteroaryl; and
any one of R′₁ to R′₁₀ is linked to L₂: 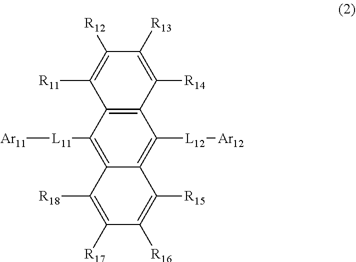
wherein,
L₁₁ and L₁₂ each independently represent a single bond, a substituted or unsubstituted (C₆-C₃₀)arylene, or a substituted or unsubstituted (3- to 30-membered)heteroarylene;
Ar₁₁ and Ar₁₂ each independently represent a substituted or unsubstituted (C₆-C₃₀)aryl or a substituted or unsubstituted (3- to 30-membered)heteroaryl; and
R₁₁ to R₁₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of (C₃-C₃₀) aliphatic ring and (C₆-C₃₀) aromatic ring.

2. The plurality of host materials according to claim 1, wherein Formula 1 is represented by any one of the following Formulas 1-1 to 1-3: 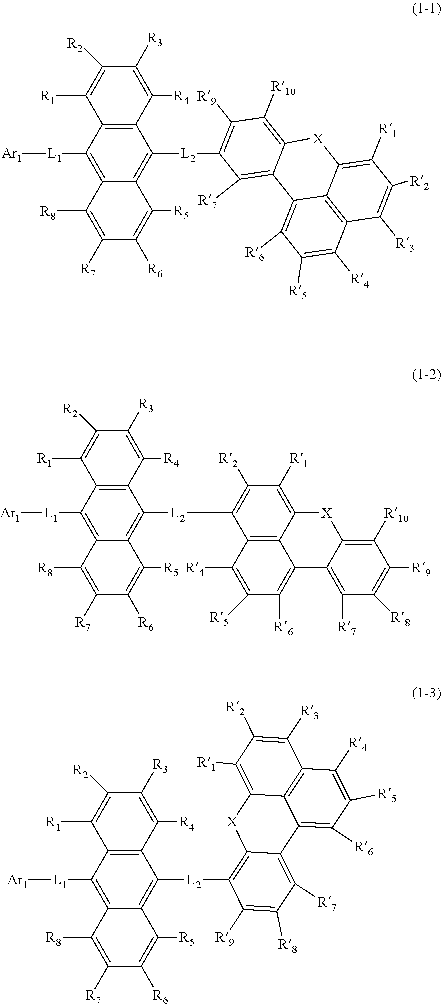
wherein,
R₁ to R₈, L₁, L₂, Ar₁, X, and R′₁ to R′₁₀ are as defined in claim 1.

3. The plurality of host materials according to claim 1, wherein in Formula 1,
R₁ to R₈ each independently represent hydrogen or deuterium; and
R′₁ to R′₁₀ each independently represent hydrogen, deuterium, a substituted or unsubstituted (C₆-C₁₅)aryl, or a substituted or unsubstituted (3- to 15-membered)heteroaryl.

4. The plurality of host materials according to claim 1, wherein in Formula 1, Ar₁ represents a substituted or unsubstituted phenyl, a substituted or unsubstituted biphenyl, a substituted or unsubstituted terphenyl, a substituted or unsubstituted naphthyl, a substituted or unsubstituted phenanthrenyl, or a combination thereof.

5. The plurality of host materials according to claim 1, wherein in Formula 2, Ar₁₁ and Ar₁₂ each independently represent a substituted or unsubstituted phenyl, a substituted or unsubstituted biphenyl, a substituted or unsubstituted terphenyl, a substituted or unsubstituted naphthyl, a substituted or unsubstituted phenanthrenyl, or the following Formula 2-A: 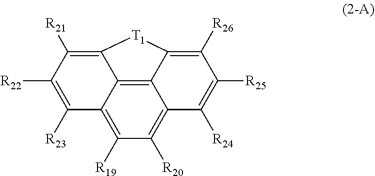
wherein,
T₁ represents —O—, —S—, or —CR₂₇R₂₈;
R₁₉ to R₂₈ each independently represent hydrogen, deuterium, a substituted or unsubstituted (C₁-C₃₀)alkyl, a substituted or unsubstituted (C₆-C₃₀)aryl, or a substituted or unsubstituted (3- to 30-membered)heteroaryl; and
any one of R₁₉ to R₂₆ is linked to L₁₁ or L₁₂.

6. The plurality of host materials according to claim 1, wherein the substituted alkyl, the substituted alkenyl, the substituted aryl(ene), the substituted heteroaryl(ene), the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted fused ring of aliphatic ring and aromatic ring, the substituted mono- or dialkylamino, the substituted mono- or dialkenylamino, the substituted mono- or diarylamino, the substituted mono- or diheteroarylamino, the substituted alkylalkenylamino, the substituted alkylarylamino, the substituted alkylheteroarylamino, the substituted alkenylarylamino, the substituted alkenylheteroarylamino, and the substituted arylheteroarylamino each independently are substituted with at least one selected from the group consisting of deuterium; halogen; cyano; carboxyl; nitro; hydroxyl; phosphine oxide; (C₁-C₃₀)alkyl unsubstituted or substituted with deuterium; halo(C₁-C₃₀)alkyl; (C₂-C₃₀)alkenyl; (C₂-C₃₀)alkynyl; (C₁-C₃₀)alkoxy; (C₁-C₃₀)alkylthio; (C₃-C₃₀)cycloalkyl; (C₃-C₃₀)cycloalkenyl; (3- to 7-membered)heterocycloalkyl; (C₆-C₃₀)aryloxy; (C₆-C₃₀)arylthio; (3- to 30-membered)heteroaryl unsubstituted or substituted with at least one of deuterium and (C₆-C₃₀)aryl; (C₆-C₃₀)aryl unsubstituted or substituted with at least one of deuterium, (C₁-C₃₀)alkyl, (C₆-C₃₀)aryl and (3- to 30-membered)heteroaryl; tri(C₁-C₃₀)alkylsilyl; tri(C₆-C₃₀)arylsilyl; di(C₁-C₃₀)alkyl(C₆-C₃₀)arylsilyl; (C₁-C₃₀)alkyldi(C₆-C₃₀)arylsilyl; amino; mono- or di-(C₁-C₃₀)alkylamino; mono- or di-(C₂-C₃₀)alkenylamino; mono- or di-(C₆-C₃₀)arylamino; mono- or di-(3- to 30-membered)heteroarylamino; (C₁-C₃₀)alkyl(C₂-C₃₀)alkenylamino; (C₁-C₃₀)alkyl(C₆-C₃₀)arylamino; (C₁-C₃₀)alkyl(3- to 30-membered)heteroarylamino; (C₂-C₃₀)alkenyl(C₆-C₃₀)arylamino; (C₂-C₃₀)alkenyl(3- to 30-membered)heteroarylamino; (C₆-C₃₀)aryl(3- to 30-membered)heteroarylamino; (C₁-C₃₀)alkylcarbonyl; (C₁-C₃₀)alkoxycarbonyl; (C₆-C₃₀)arylcarbonyl; di(C₆-C₃₀)arylboronyl; (C₆-C₃₀)arylphosphonyl; di(C₁-C₃₀)alkylboronyl; (C₁-C₃₀)alkyl(C₆-C₃₀)arylboronyl; (C₆-C₃₀)ar(C₁-C₃₀)alkyl; and (C₁-C₃₀)alkyl(C₆-C₃₀)aryl.

7. The plurality of host materials according to claim 1, wherein the first host compound represented by Formula 1 is selected from the following compounds: 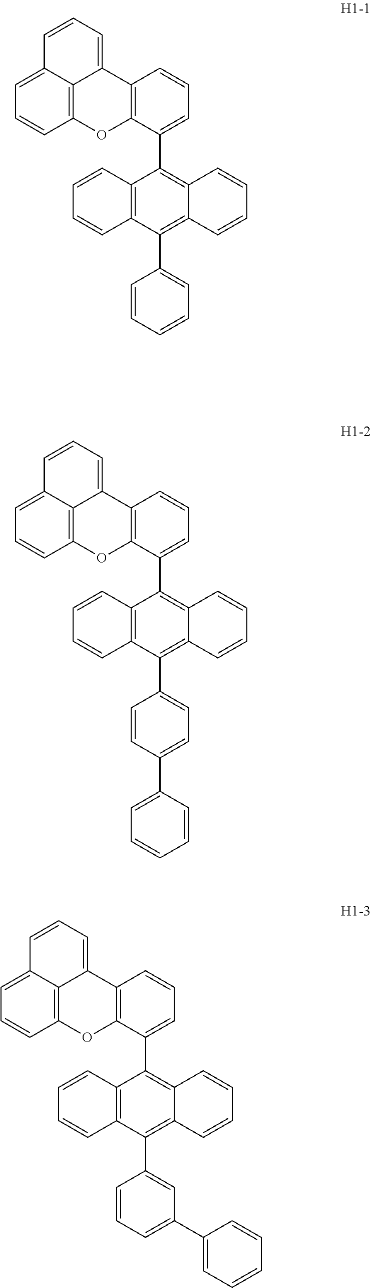 [SMILES: c1ccc(-c2c3ccccc3c(-c3cccc4c3Oc3cccc5cccc-4c35)c3ccccc23)cc1 ; c1ccc(-c2ccc(-c3c4ccccc4c(-c4cccc5c4Oc4cccc6cccc-5c46)c4ccccc34)cc2)cc1 ; c1ccc(-c2cccc(-c3c4ccccc4c(-c4cccc5c4Oc4cccc6cccc-5c46)c4ccccc34)c2)cc1] 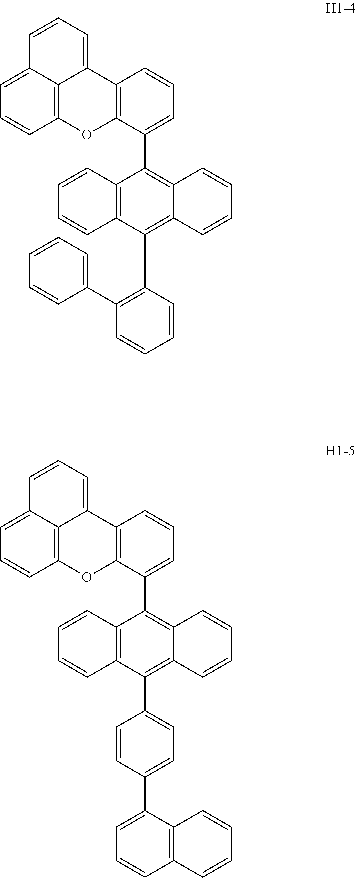 [SMILES: c1ccc(-c2ccccc2-c2c3ccccc3c(-c3cccc4c3Oc3cccc5cccc-4c35)c3ccccc23)cc1 ; c1cc(-c2c3ccccc3c(-c3ccc(-c4cccc5ccccc45)cc3)c3ccccc23)c2c(c1)-c1cccc3cccc(c13)O2] 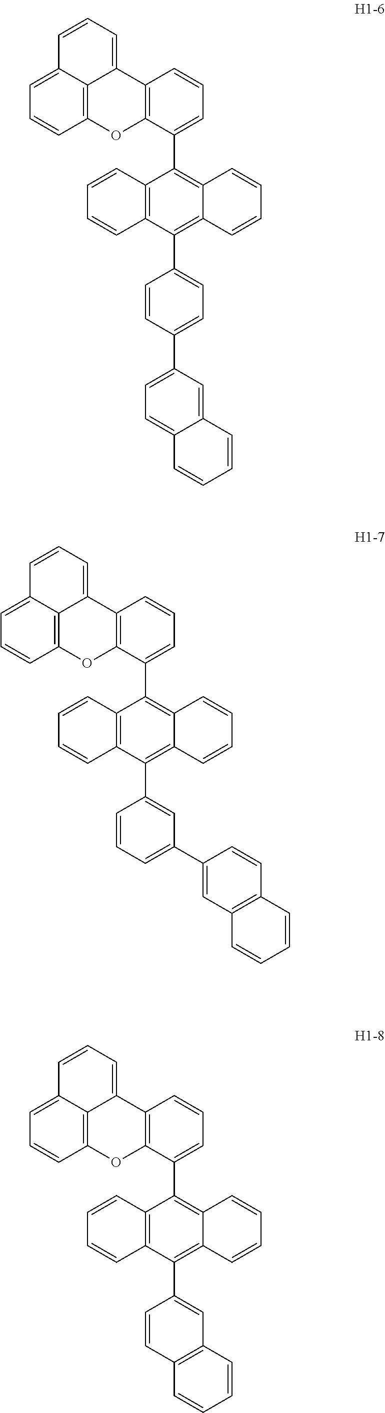 [SMILES: c1cc(-c2c3ccccc3c(-c3ccc(-c4ccc5ccccc5c4)cc3)c3ccccc23)c2c(c1)-c1cccc3cccc(c13)O2 ; c1cc(-c2ccc3ccccc3c2)cc(-c2c3ccccc3c(-c3cccc4c3Oc3cccc5cccc-4c35)c3ccccc23)c1 ; c1cc(-c2c3ccccc3c(-c3ccc4ccccc4c3)c3ccccc23)c2c(c1)-c1cccc3cccc(c13)O2] 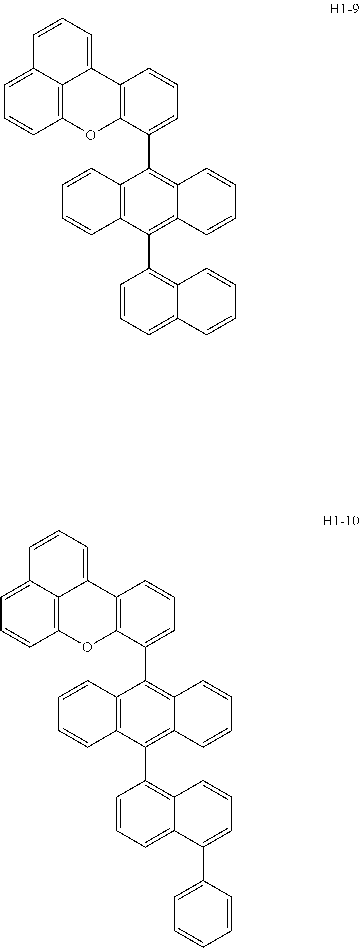 [SMILES: c1cc(-c2c3ccccc3c(-c3cccc4ccccc34)c3ccccc23)c2c(c1)-c1cccc3cccc(c13)O2 ; c1ccc(-c2cccc3c(-c4c5ccccc5c(-c5cccc6c5Oc5cccc7cccc-6c57)c5ccccc45)cccc23)cc1] 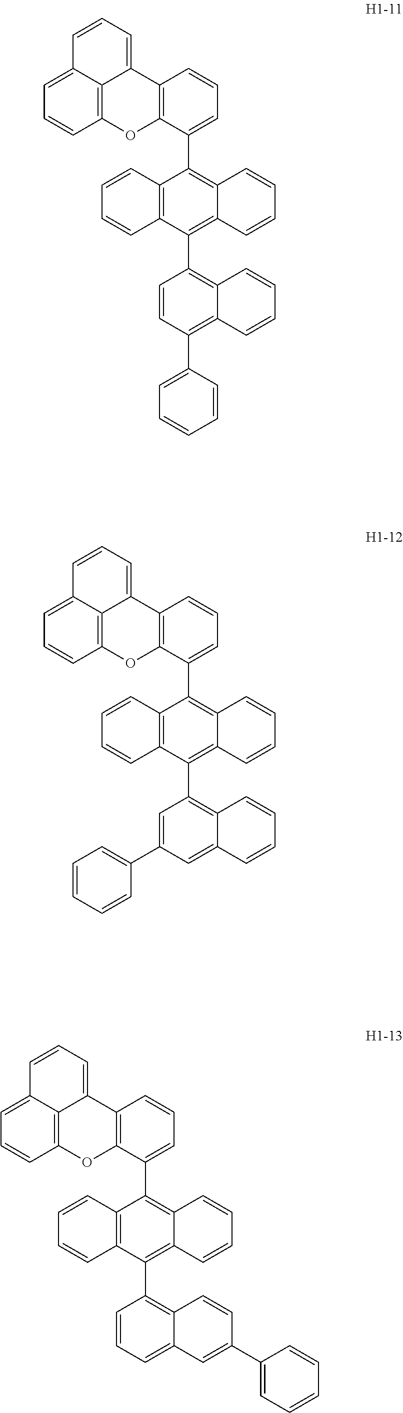 [SMILES: c1ccc(-c2ccc(-c3c4ccccc4c(-c4cccc5c4Oc4cccc6cccc-5c46)c4ccccc34)c3ccccc23)cc1 ; c1ccc(-c2cc(-c3c4ccccc4c(-c4cccc5c4Oc4cccc6cccc-5c46)c4ccccc34)c3ccccc3c2)cc1 ; c1ccc(-c2ccc3c(-c4c5ccccc5c(-c5cccc6c5Oc5cccc7cccc-6c57)c5ccccc45)cccc3c2)cc1] 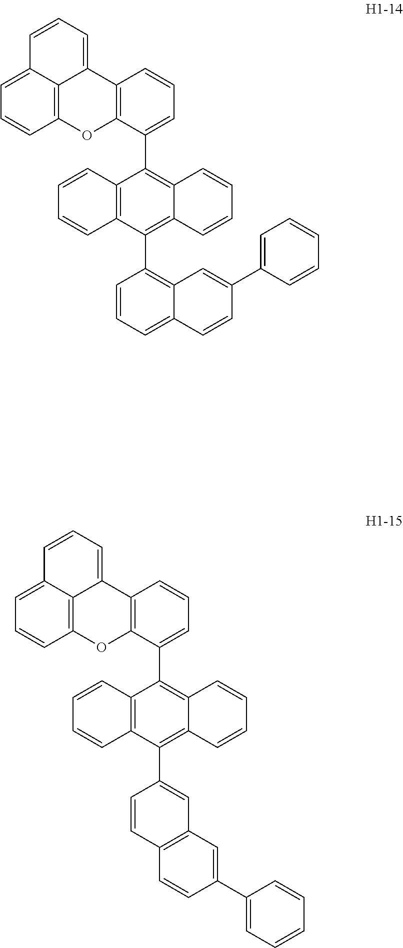 [SMILES: c1ccc(-c2ccc3cccc(-c4c5ccccc5c(-c5cccc6c5Oc5cccc7cccc-6c57)c5ccccc45)c3c2)cc1 ; c1ccc(-c2ccc3ccc(-c4c5ccccc5c(-c5cccc6c5Oc5cccc7cccc-6c57)c5ccccc45)cc3c2)cc1] 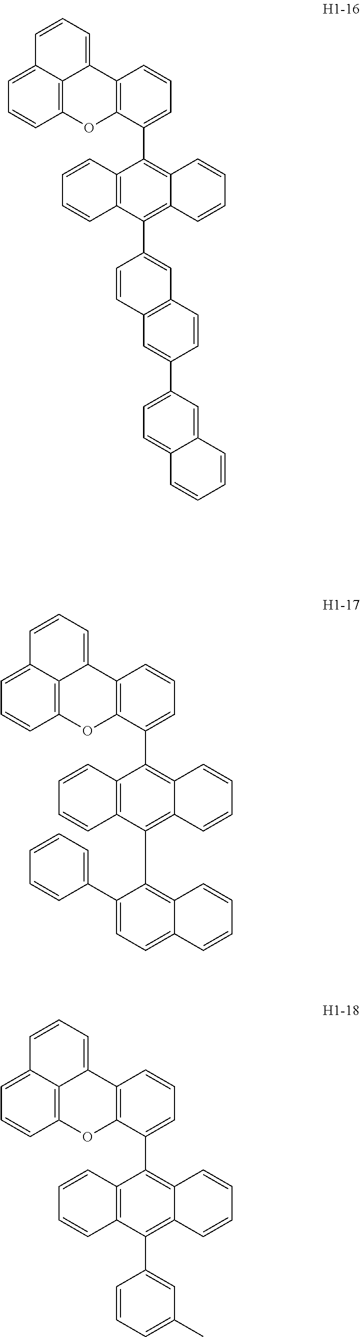 [SMILES: c1cc(-c2c3ccccc3c(-c3ccc4cc(-c5ccc6ccccc6c5)ccc4c3)c3ccccc23)c2c(c1)-c1cccc3cccc(c13)O2 ; c1ccc(-c2ccc3ccccc3c2-c2c3ccccc3c(-c3cccc4c3Oc3cccc5cccc-4c35)c3ccccc23)cc1 ; Cc1cccc(-c2c3ccccc3c(-c3cccc4c3Oc3cccc5cccc-4c35)c3ccccc23)c1] 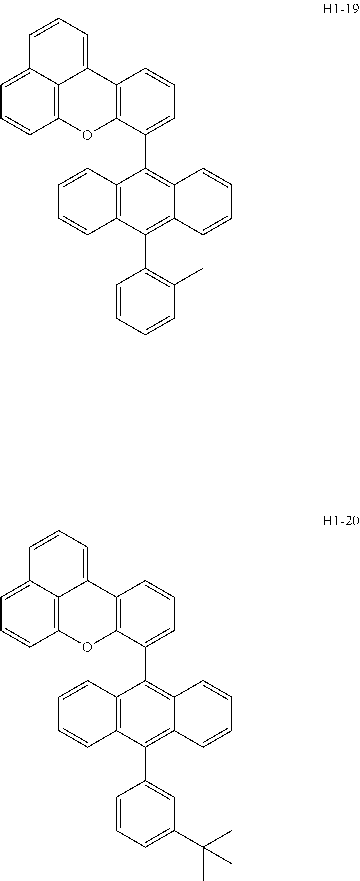 [SMILES: Cc1ccccc1-c1c2ccccc2c(-c2cccc3c2Oc2cccc4cccc-3c24)c2ccccc12 ; CC(C)(C)c1cccc(-c2c3ccccc3c(-c3cccc4c3Oc3cccc5cccc-4c35)c3ccccc23)c1] 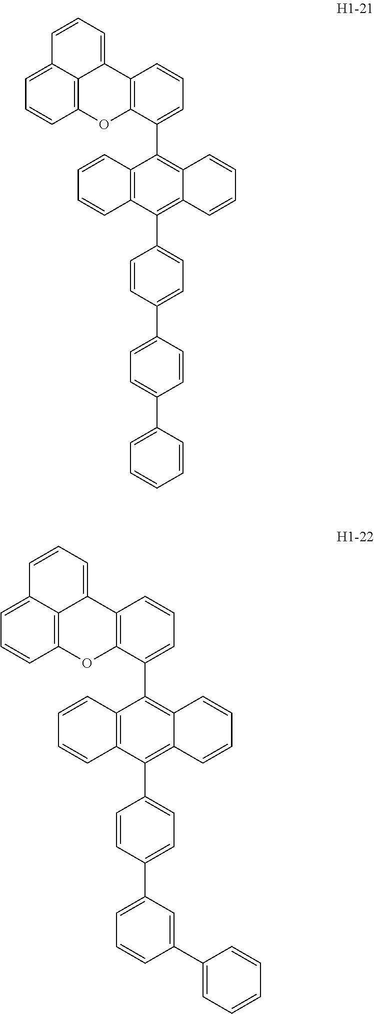 [SMILES: c1ccc(-c2ccc(-c3ccc(-c4c5ccccc5c(-c5cccc6c5Oc5cccc7cccc-6c57)c5ccccc45)cc3)cc2)cc1 ; c1ccc(-c2cccc(-c3ccc(-c4c5ccccc5c(-c5cccc6c5Oc5cccc7cccc-6c57)c5ccccc45)cc3)c2)cc1] 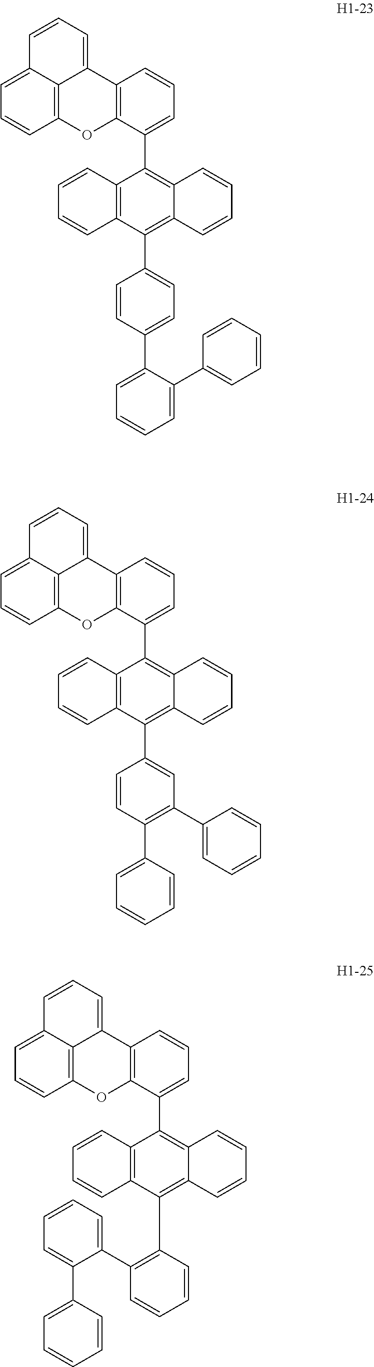 [SMILES: c1ccc(-c2ccccc2-c2ccc(-c3c4ccccc4c(-c4cccc5c4Oc4cccc6cccc-5c46)c4ccccc34)cc2)cc1 ; c1ccc(-c2ccc(-c3c4ccccc4c(-c4cccc5c4Oc4cccc6cccc-5c46)c4ccccc34)cc2-c2ccccc2)cc1 ; c1ccc(-c2ccccc2-c2ccccc2-c2c3ccccc3c(-c3cccc4c3Oc3cccc5cccc-4c35)c3ccccc23)cc1] 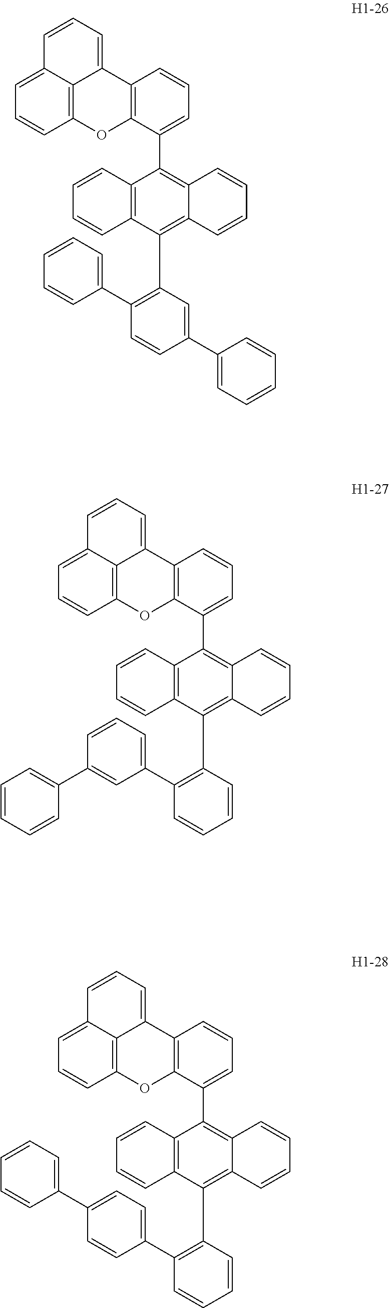 [SMILES: c1ccc(-c2ccc(-c3ccccc3)c(-c3c4ccccc4c(-c4cccc5c4Oc4cccc6cccc-5c46)c4ccccc34)c2)cc1 ; c1ccc(-c2cccc(-c3ccccc3-c3c4ccccc4c(-c4cccc5c4Oc4cccc6cccc-5c46)c4ccccc34)c2)cc1 ; c1ccc(-c2ccc(-c3ccccc3-c3c4ccccc4c(-c4cccc5c4Oc4cccc6cccc-5c46)c4ccccc34)cc2)cc1] 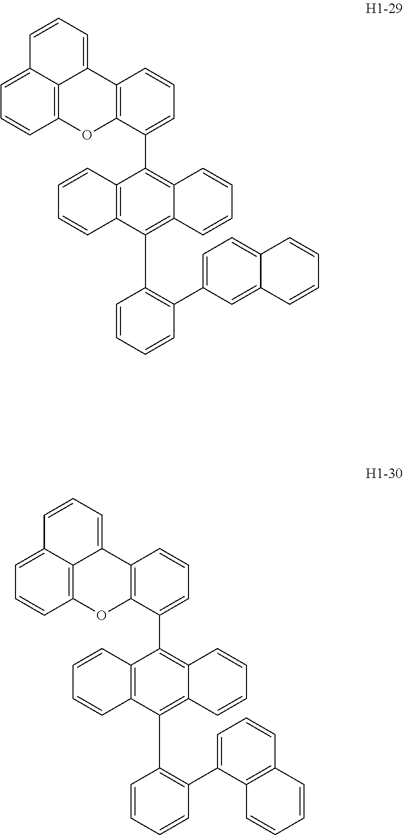 [SMILES: c1ccc(-c2c3ccccc3c(-c3cccc4c3Oc3cccc5cccc-4c35)c3ccccc23)c(-c2ccc3ccccc3c2)c1 ; c1ccc(-c2c3ccccc3c(-c3cccc4c3Oc3cccc5cccc-4c35)c3ccccc23)c(-c2cccc3ccccc23)c1] 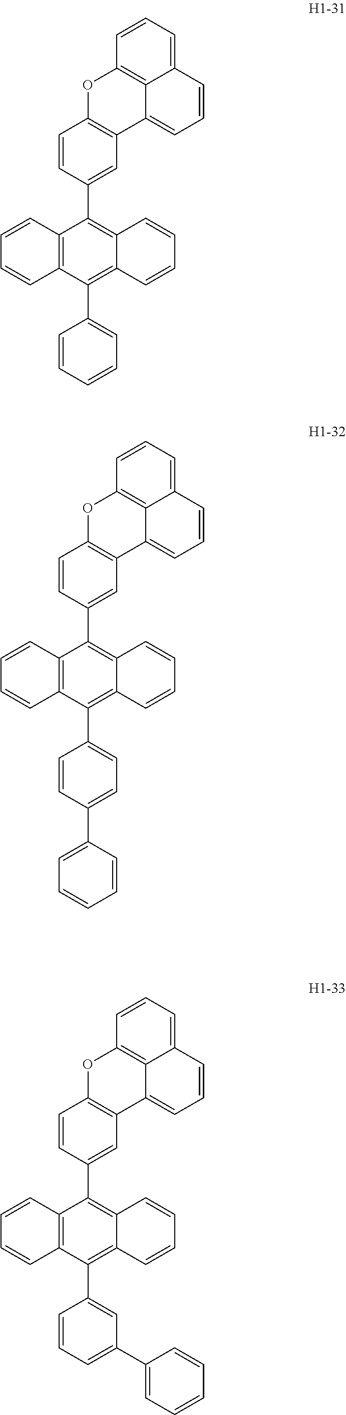 [SMILES: c1ccc(-c2c3ccccc3c(-c3ccc4c(c3)-c3cccc5cccc(c35)O4)c3ccccc23)cc1 ; c1ccc(-c2ccc(-c3c4ccccc4c(-c4ccc5c(c4)-c4cccc6cccc(c46)O5)c4ccccc34)cc2)cc1 ; c1ccc(-c2cccc(-c3c4ccccc4c(-c4ccc5c(c4)-c4cccc6cccc(c46)O5)c4ccccc34)c2)cc1] 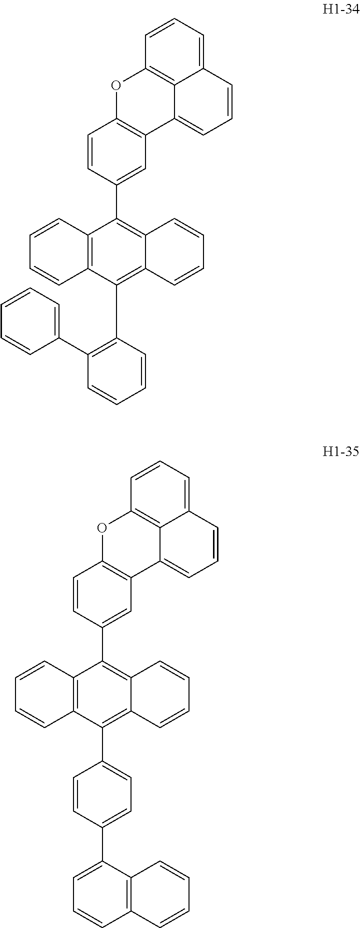 [SMILES: c1ccc(-c2ccccc2-c2c3ccccc3c(-c3ccc4c(c3)-c3cccc5cccc(c35)O4)c3ccccc23)cc1 ; c1ccc2c(-c3ccc(-c4c5ccccc5c(-c5ccc6c(c5)-c5cccc7cccc(c57)O6)c5ccccc45)cc3)cccc2c1] 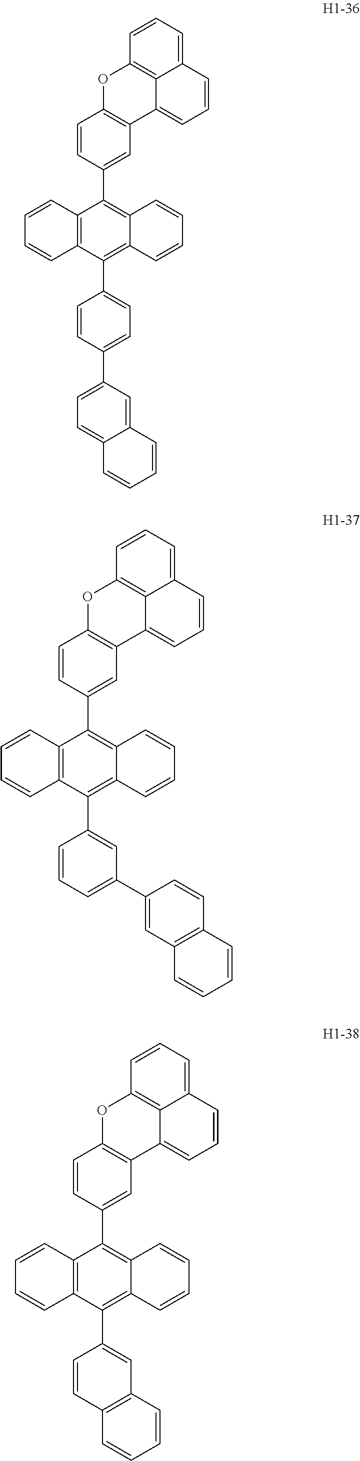 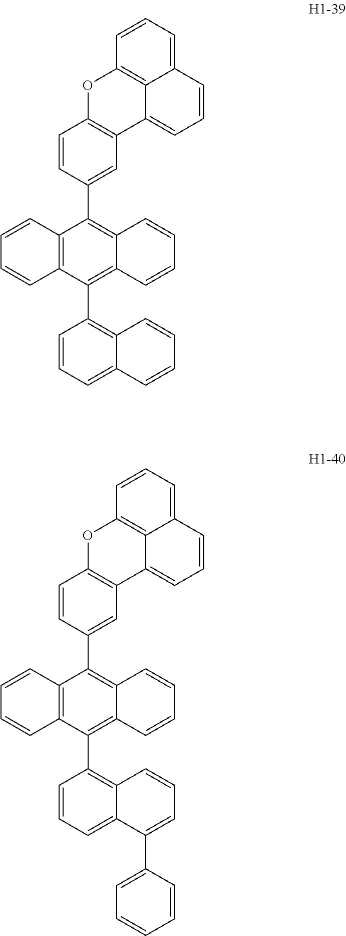 [SMILES: c1ccc2c(-c3c4ccccc4c(-c4ccc5c(c4)-c4cccc6cccc(c46)O5)c4ccccc34)cccc2c1 ; c1ccc(-c2cccc3c(-c4c5ccccc5c(-c5ccc6c(c5)-c5cccc7cccc(c57)O6)c5ccccc45)cccc23)cc1] 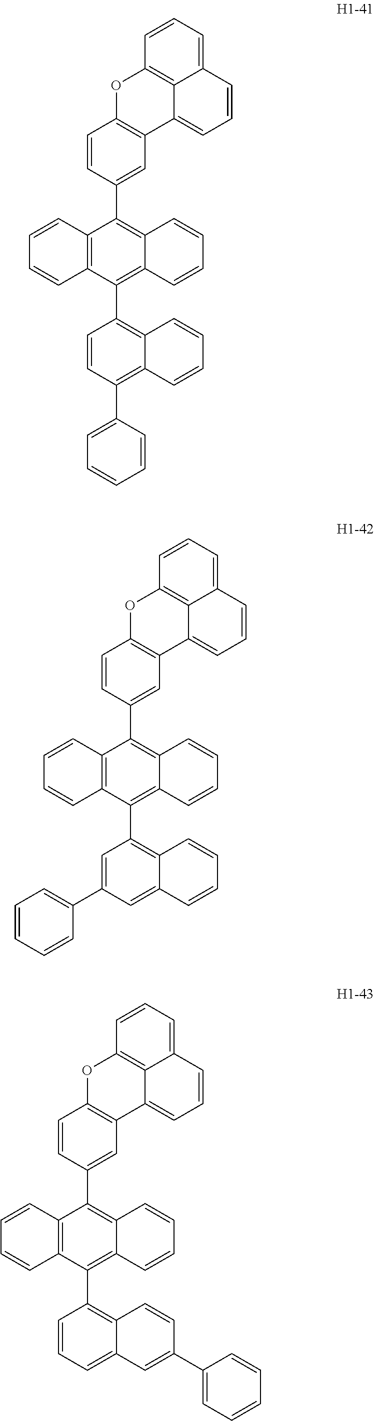 [SMILES: c1ccc(-c2ccc(-c3c4ccccc4c(-c4ccc5c(c4)-c4cccc6cccc(c46)O5)c4ccccc34)c3ccccc23)cc1 ; c1ccc(-c2cc(-c3c4ccccc4c(-c4ccc5c(c4)-c4cccc6cccc(c46)O5)c4ccccc34)c3ccccc3c2)cc1 ; c1ccc(-c2ccc3c(-c4c5ccccc5c(-c5ccc6c(c5)-c5cccc7cccc(c57)O6)c5ccccc45)cccc3c2)cc1] 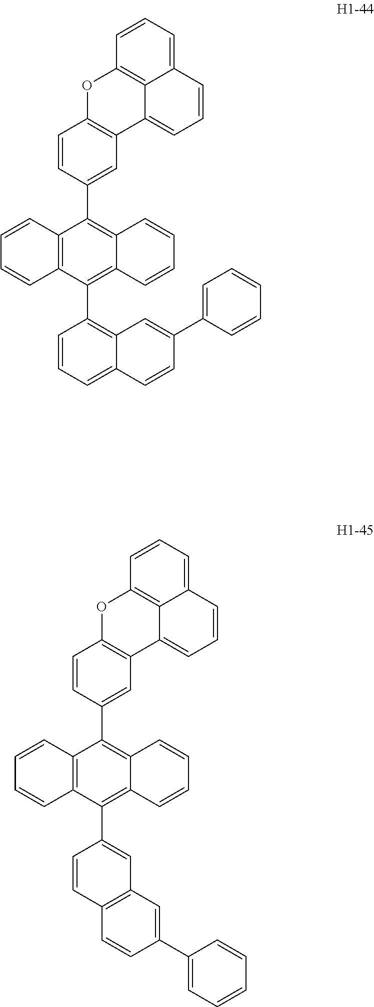 [SMILES: c1ccc(-c2ccc3cccc(-c4c5ccccc5c(-c5ccc6c(c5)-c5cccc7cccc(c57)O6)c5ccccc45)c3c2)cc1 ; c1ccc(-c2ccc3ccc(-c4c5ccccc5c(-c5ccc6c(c5)-c5cccc7cccc(c57)O6)c5ccccc45)cc3c2)cc1] 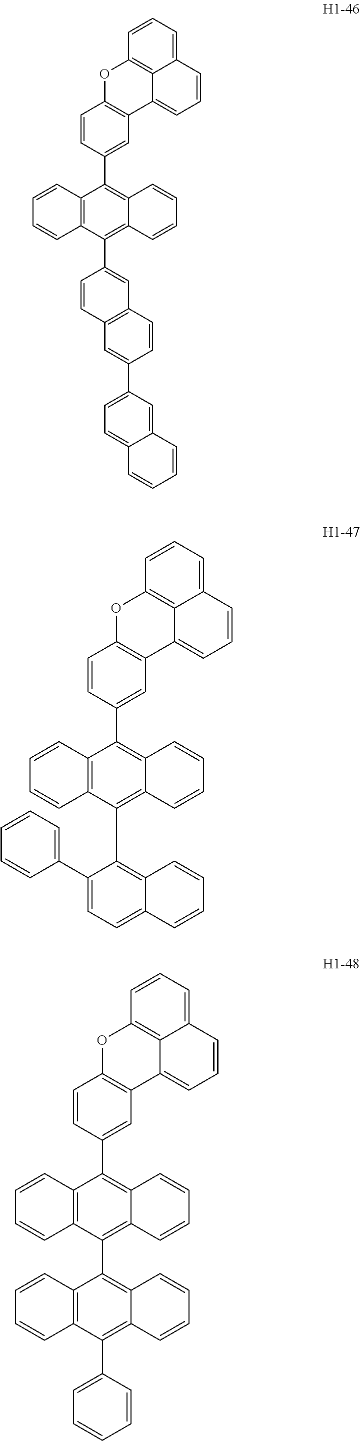 [SMILES: c1ccc2cc(-c3ccc4cc(-c5c6ccccc6c(-c6ccc7c(c6)-c6cccc8cccc(c68)O7)c6ccccc56)ccc4c3)ccc2c1 ; c1ccc(-c2ccc3ccccc3c2-c2c3ccccc3c(-c3ccc4c(c3)-c3cccc5cccc(c35)O4)c3ccccc23)cc1 ; c1ccc(-c2c3ccccc3c(-c3c4ccccc4c(-c4ccc5c(c4)-c4cccc6cccc(c46)O5)c4ccccc34)c3ccccc23)cc1] 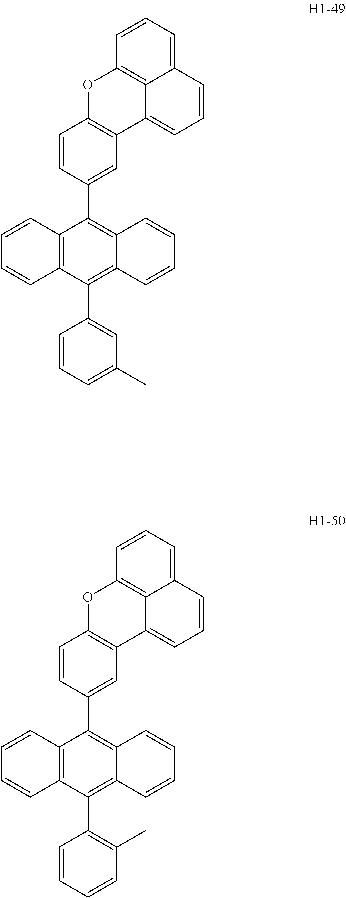 [SMILES: Cc1cccc(-c2c3ccccc3c(-c3ccc4c(c3)-c3cccc5cccc(c35)O4)c3ccccc23)c1 ; Cc1ccccc1-c1c2ccccc2c(-c2ccc3c(c2)-c2cccc4cccc(c24)O3)c2ccccc12] 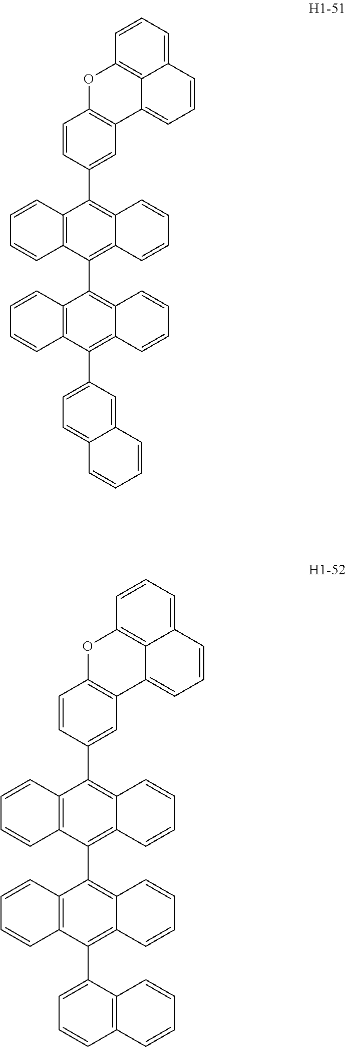 [SMILES: c1ccc2cc(-c3c4ccccc4c(-c4c5ccccc5c(-c5ccc6c(c5)-c5cccc7cccc(c57)O6)c5ccccc45)c4ccccc34)ccc2c1 ; c1ccc2c(-c3c4ccccc4c(-c4c5ccccc5c(-c5ccc6c(c5)-c5cccc7cccc(c57)O6)c5ccccc45)c4ccccc34)cccc2c1] 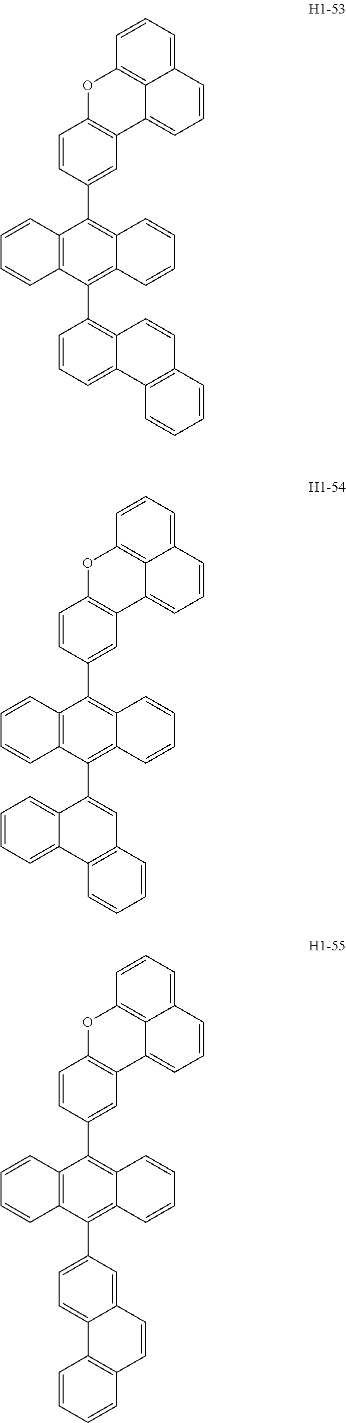 [SMILES: c1cc2c3c(cccc3c1)-c1cc(-c3c4ccccc4c(-c4cccc5c4ccc4ccccc45)c4ccccc34)ccc1O2 ; c1cc2c3c(cccc3c1)-c1cc(-c3c4ccccc4c(-c4cc5ccccc5c5ccccc45)c4ccccc34)ccc1O2 ; c1cc2c3c(cccc3c1)-c1cc(-c3c4ccccc4c(-c4ccc5c(ccc6ccccc65)c4)c4ccccc34)ccc1O2] 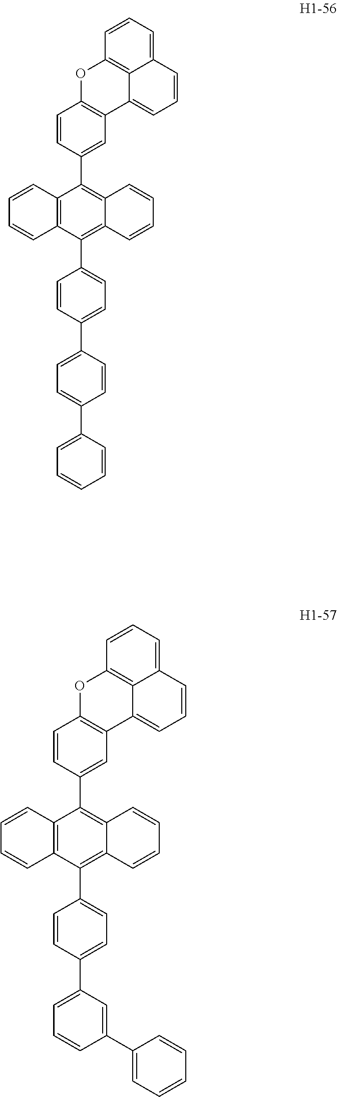 [SMILES: c1ccc(-c2ccc(-c3ccc(-c4c5ccccc5c(-c5ccc6c(c5)-c5cccc7cccc(c57)O6)c5ccccc45)cc3)cc2)cc1 ; c1ccc(-c2cccc(-c3ccc(-c4c5ccccc5c(-c5ccc6c(c5)-c5cccc7cccc(c57)O6)c5ccccc45)cc3)c2)cc1] 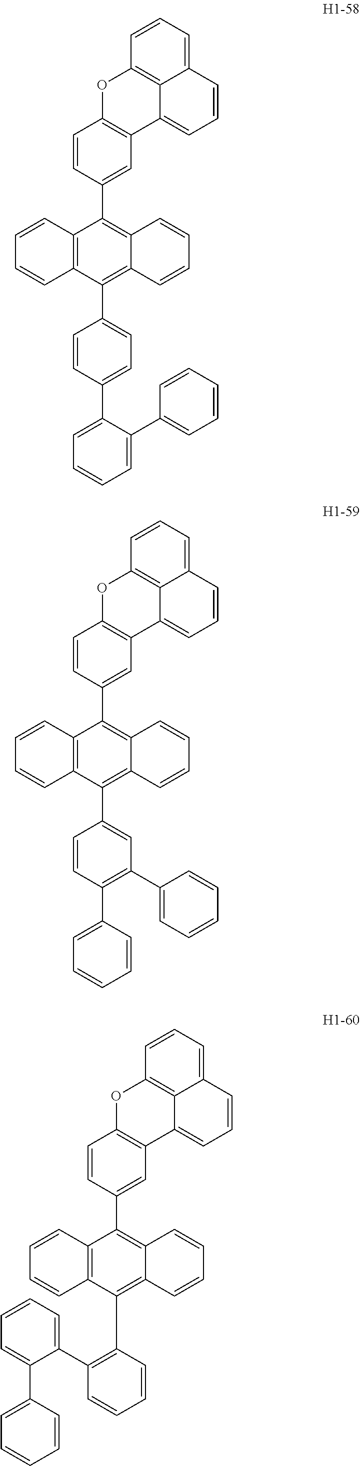 [SMILES: c1ccc(-c2ccccc2-c2ccc(-c3c4ccccc4c(-c4ccc5c(c4)-c4cccc6cccc(c46)O5)c4ccccc34)cc2)cc1 ; c1ccc(-c2ccc(-c3c4ccccc4c(-c4ccc5c(c4)-c4cccc6cccc(c46)O5)c4ccccc34)cc2-c2ccccc2)cc1 ; c1ccc(-c2ccccc2-c2ccccc2-c2c3ccccc3c(-c3ccc4c(c3)-c3cccc5cccc(c35)O4)c3ccccc23)cc1] 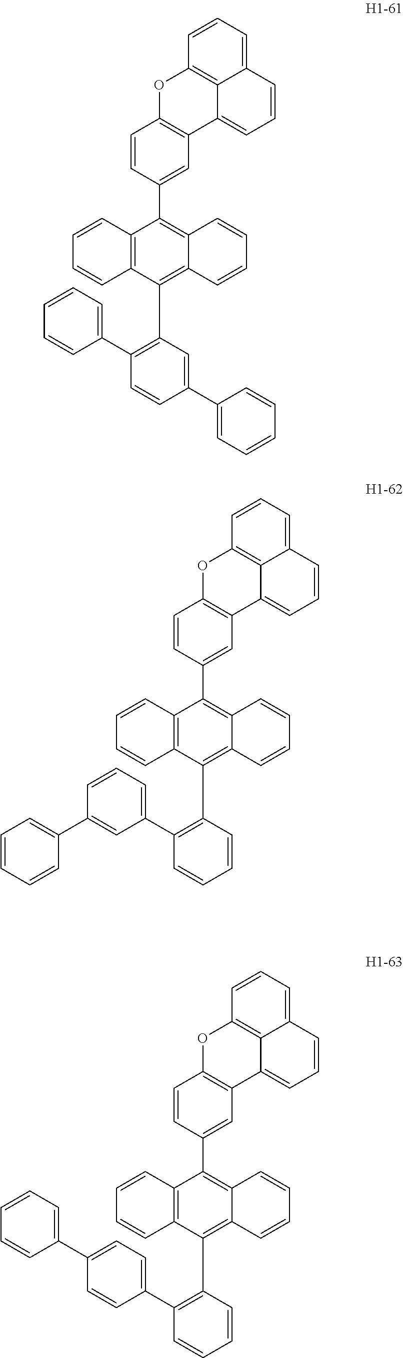 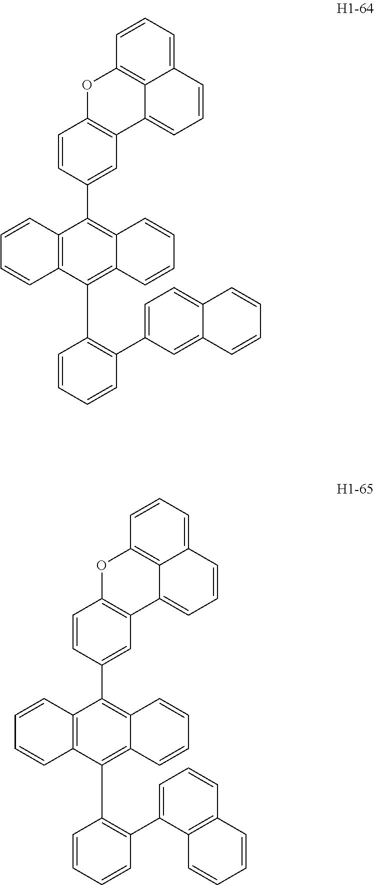 [SMILES: c1ccc(-c2c3ccccc3c(-c3ccc4c(c3)-c3cccc5cccc(c35)O4)c3ccccc23)c(-c2ccc3ccccc3c2)c1 ; c1ccc(-c2c3ccccc3c(-c3ccc4c(c3)-c3cccc5cccc(c35)O4)c3ccccc23)c(-c2cccc3ccccc23)c1] 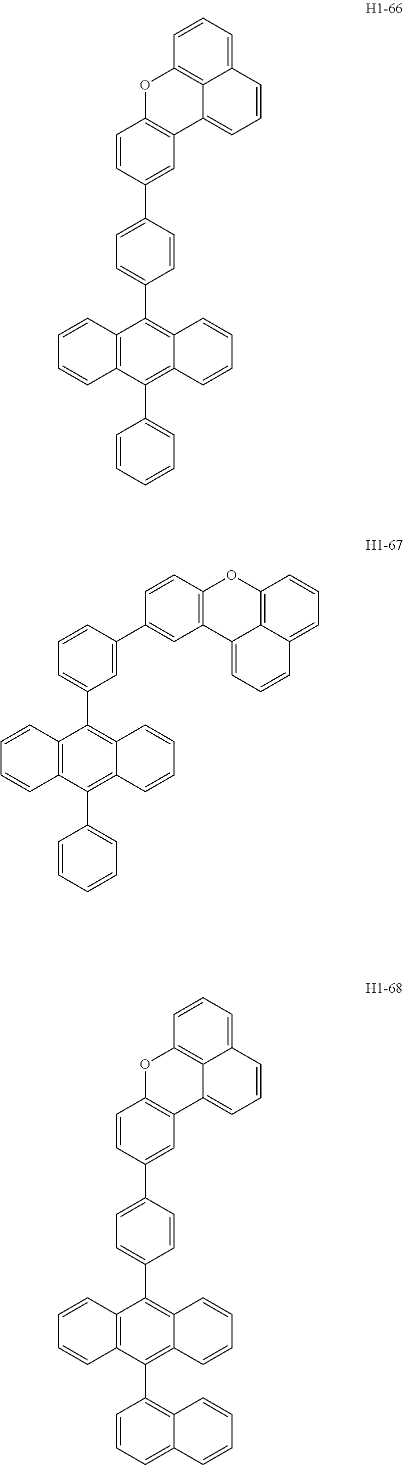 [SMILES: c1ccc(-c2c3ccccc3c(-c3ccc(-c4ccc5c(c4)-c4cccc6cccc(c46)O5)cc3)c3ccccc23)cc1 ; c1ccc(-c2c3ccccc3c(-c3cccc(-c4ccc5c(c4)-c4cccc6cccc(c46)O5)c3)c3ccccc23)cc1 ; c1ccc2c(-c3c4ccccc4c(-c4ccc(-c5ccc6c(c5)-c5cccc7cccc(c57)O6)cc4)c4ccccc34)cccc2c1] 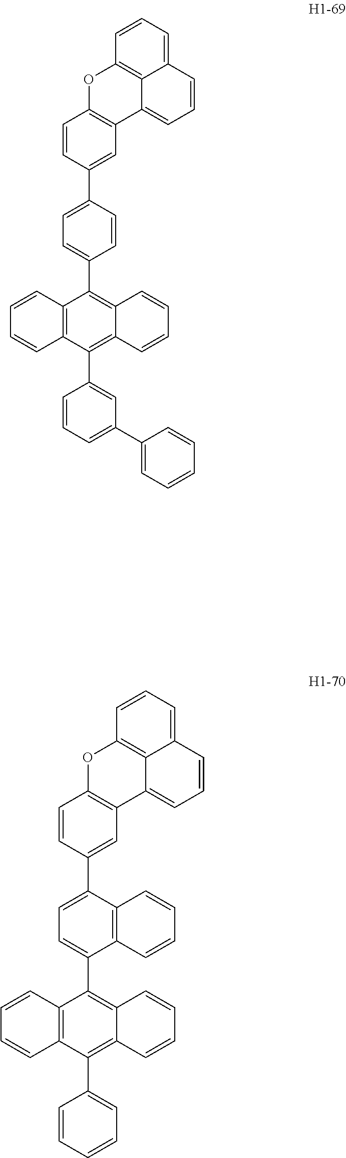 [SMILES: c1ccc(-c2cccc(-c3c4ccccc4c(-c4ccc(-c5ccc6c(c5)-c5cccc7cccc(c57)O6)cc4)c4ccccc34)c2)cc1 ; c1ccc(-c2c3ccccc3c(-c3ccc(-c4ccc5c(c4)-c4cccc6cccc(c46)O5)c4ccccc34)c3ccccc23)cc1] 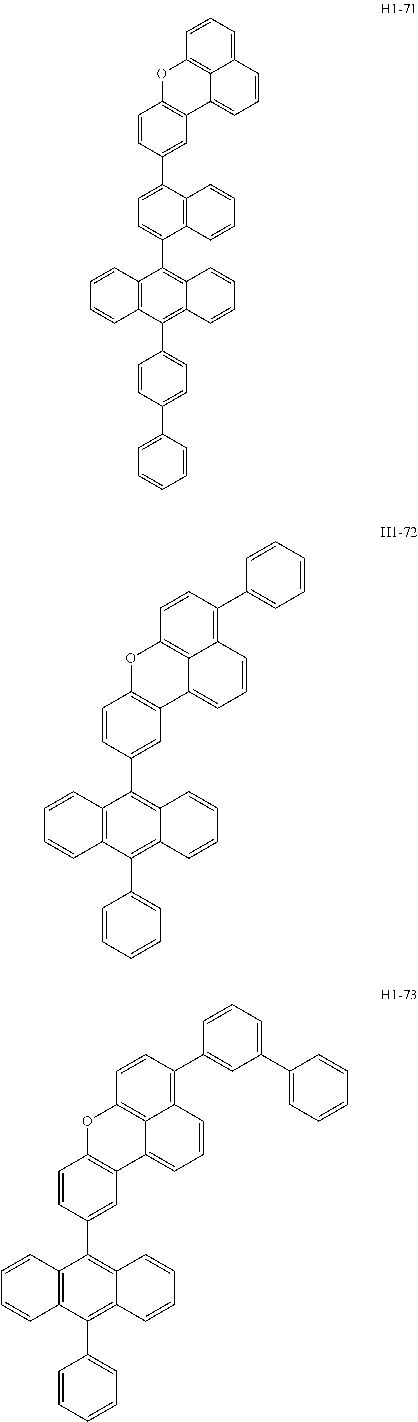 [SMILES: c1ccc(-c2ccc(-c3c4ccccc4c(-c4ccc(-c5ccc6c(c5)-c5cccc7cccc(c57)O6)c5ccccc45)c4ccccc34)cc2)cc1 ; c1ccc(-c2c3ccccc3c(-c3ccc4c(c3)-c3cccc5c(-c6ccccc6)ccc(c35)O4)c3ccccc23)cc1 ; c1ccc(-c2cccc(-c3ccc4c5c(cccc35)-c3cc(-c5c6ccccc6c(-c6ccccc6)c6ccccc56)ccc3O4)c2)cc1] 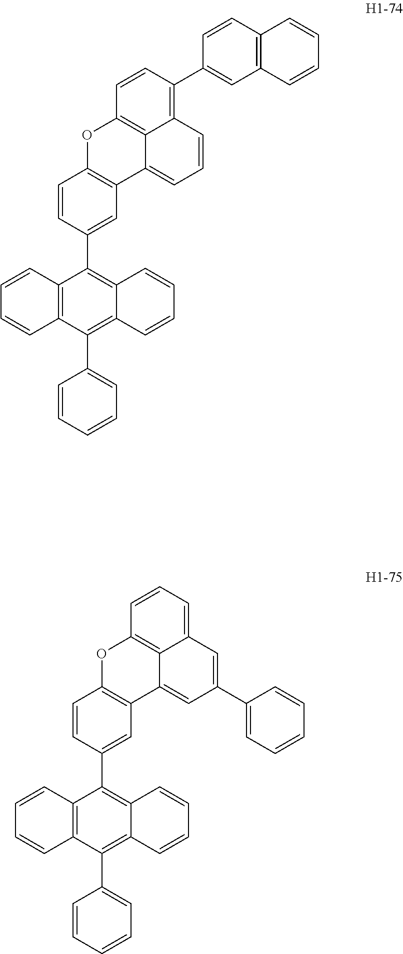 [SMILES: c1ccc(-c2c3ccccc3c(-c3ccc4c(c3)-c3cccc5c(-c6ccc7ccccc7c6)ccc(c35)O4)c3ccccc23)cc1 ; c1ccc(-c2cc3c4c(cccc4c2)Oc2ccc(-c4c5ccccc5c(-c5ccccc5)c5ccccc45)cc2-3)cc1] 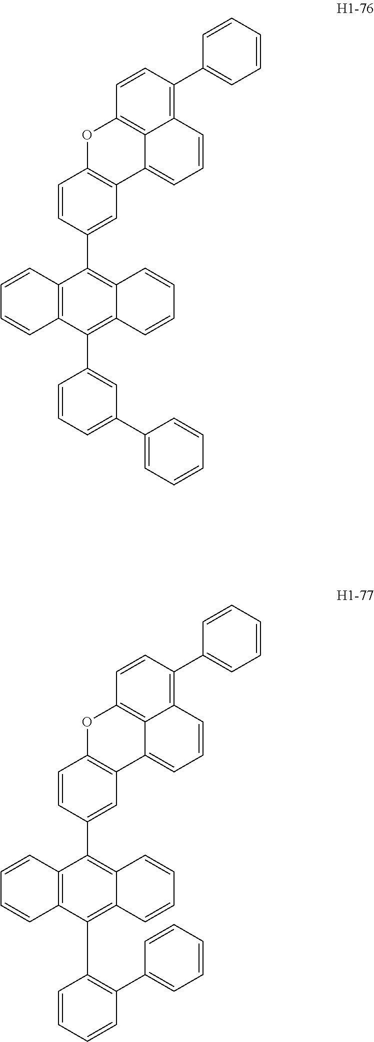 [SMILES: c1ccc(-c2cccc(-c3c4ccccc4c(-c4ccc5c(c4)-c4cccc6c(-c7ccccc7)ccc(c46)O5)c4ccccc34)c2)cc1 ; c1ccc(-c2ccccc2-c2c3ccccc3c(-c3ccc4c(c3)-c3cccc5c(-c6ccccc6)ccc(c35)O4)c3ccccc23)cc1] 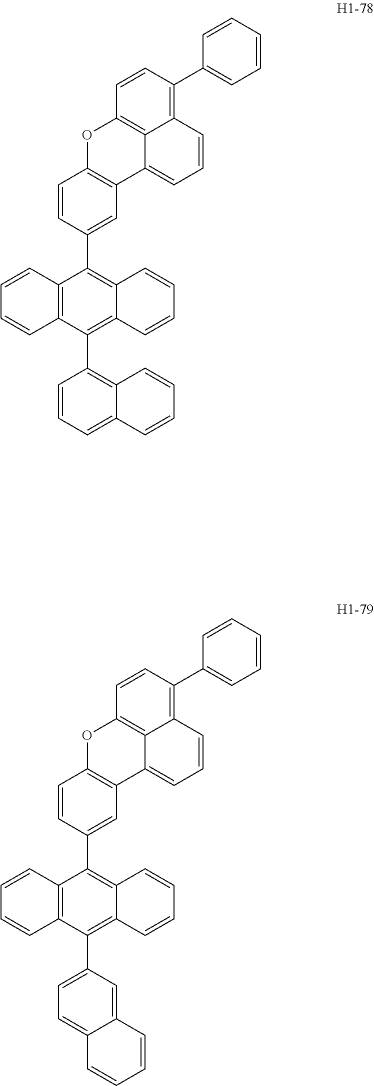 [SMILES: c1ccc(-c2ccc3c4c(cccc24)-c2cc(-c4c5ccccc5c(-c5cccc6ccccc56)c5ccccc45)ccc2O3)cc1 ; c1ccc(-c2ccc3c4c(cccc24)-c2cc(-c4c5ccccc5c(-c5ccc6ccccc6c5)c5ccccc45)ccc2O3)cc1] 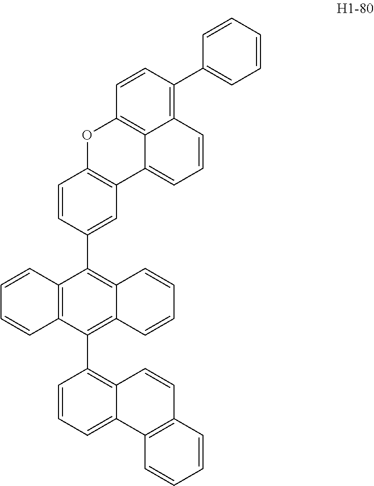 [SMILES: c1ccc(-c2ccc3c4c(cccc24)-c2cc(-c4c5ccccc5c(-c5cccc6c5ccc5ccccc56)c5ccccc45)ccc2O3)cc1] 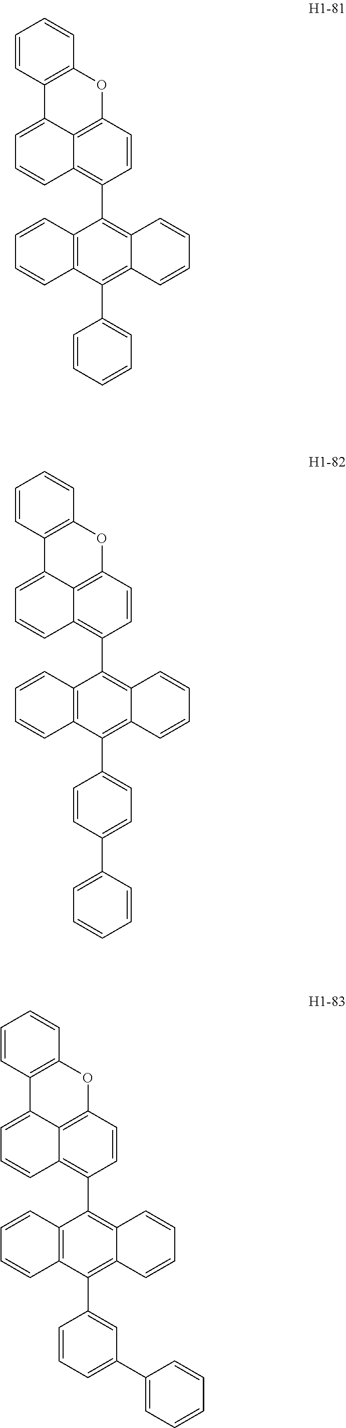 [SMILES: c1ccc(-c2c3ccccc3c(-c3ccc4c5c(cccc35)-c3ccccc3O4)c3ccccc23)cc1 ; c1ccc(-c2ccc(-c3c4ccccc4c(-c4ccc5c6c(cccc46)-c4ccccc4O5)c4ccccc34)cc2)cc1 ; c1ccc(-c2cccc(-c3c4ccccc4c(-c4ccc5c6c(cccc46)-c4ccccc4O5)c4ccccc34)c2)cc1] 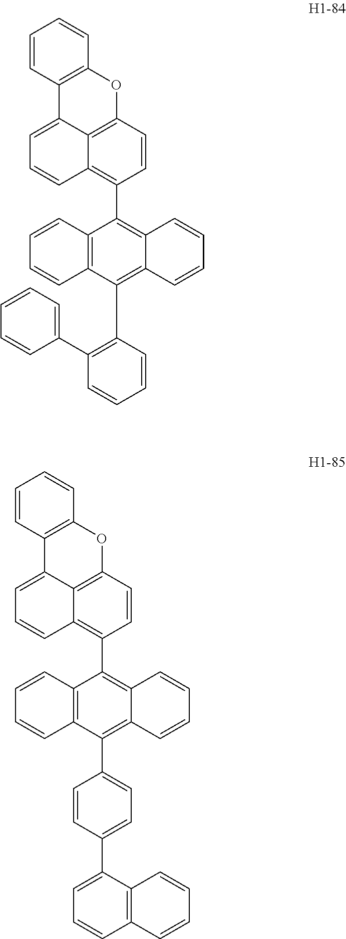 [SMILES: c1ccc(-c2ccccc2-c2c3ccccc3c(-c3ccc4c5c(cccc35)-c3ccccc3O4)c3ccccc23)cc1 ; c1ccc2c(c1)Oc1ccc(-c3c4ccccc4c(-c4ccc(-c5cccc6ccccc56)cc4)c4ccccc34)c3cccc-2c13] 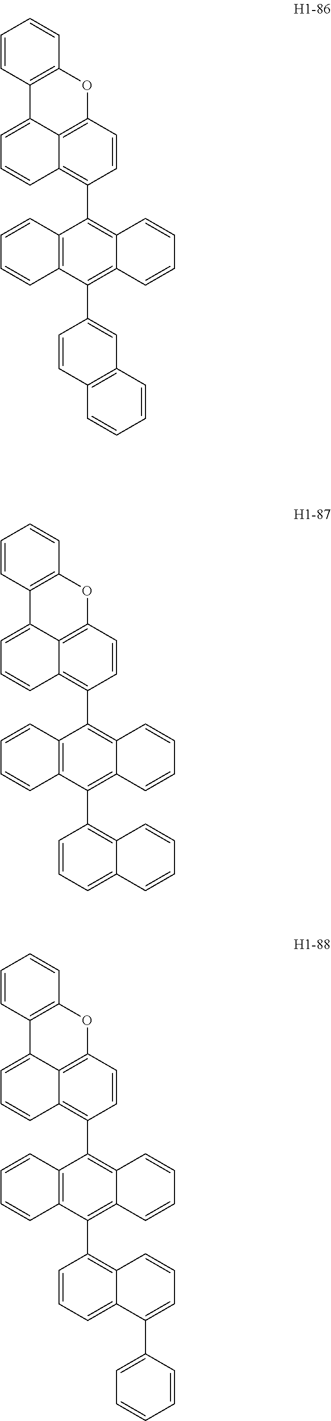 [SMILES: c1ccc2c(c1)Oc1ccc(-c3c4ccccc4c(-c4ccc5ccccc5c4)c4ccccc34)c3cccc-2c13 ; c1ccc2c(c1)Oc1ccc(-c3c4ccccc4c(-c4cccc5ccccc45)c4ccccc34)c3cccc-2c13 ; c1ccc(-c2cccc3c(-c4c5ccccc5c(-c5ccc6c7c(cccc57)-c5ccccc5O6)c5ccccc45)cccc23)cc1] 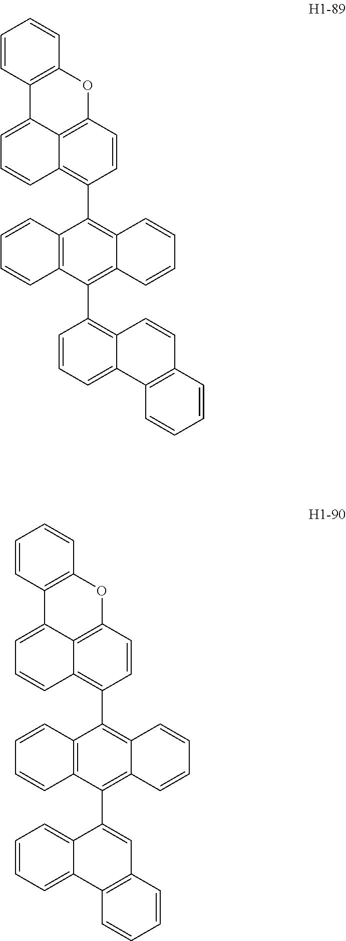 [SMILES: c1ccc2c(c1)Oc1ccc(-c3c4ccccc4c(-c4cccc5c4ccc4ccccc45)c4ccccc34)c3cccc-2c13 ; c1ccc2c(c1)Oc1ccc(-c3c4ccccc4c(-c4cc5ccccc5c5ccccc45)c4ccccc34)c3cccc-2c13] 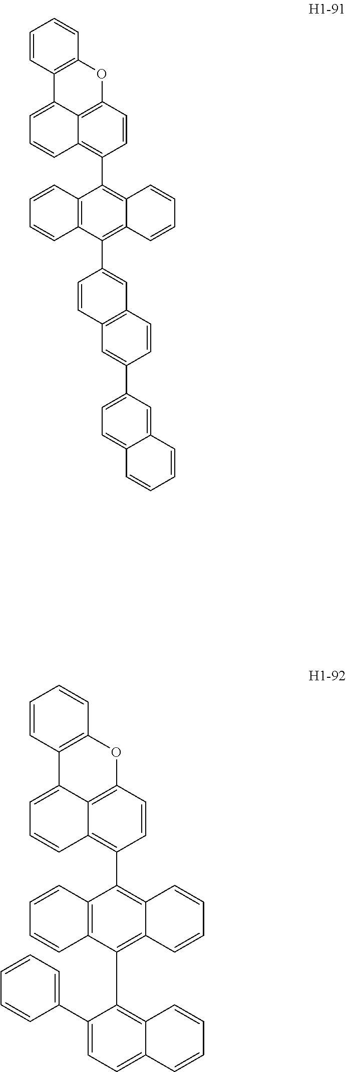 [SMILES: c1ccc2c(c1)Oc1ccc(-c3c4ccccc4c(-c4ccc5cc(-c6ccc7ccccc7c6)ccc5c4)c4ccccc34)c3cccc-2c13 ; c1ccc(-c2ccc3ccccc3c2-c2c3ccccc3c(-c3ccc4c5c(cccc35)-c3ccccc3O4)c3ccccc23)cc1] 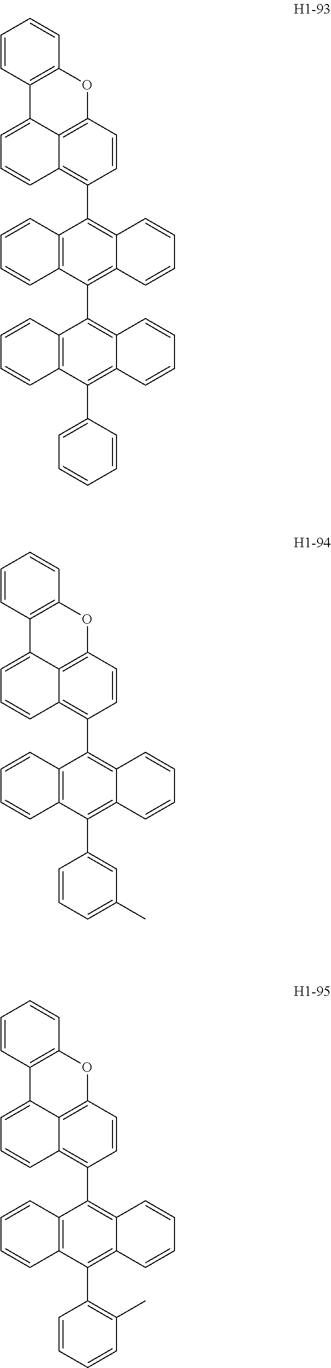 [SMILES: c1ccc(-c2c3ccccc3c(-c3c4ccccc4c(-c4ccc5c6c(cccc46)-c4ccccc4O5)c4ccccc34)c3ccccc23)cc1 ; Cc1cccc(-c2c3ccccc3c(-c3ccc4c5c(cccc35)-c3ccccc3O4)c3ccccc23)c1 ; Cc1ccccc1-c1c2ccccc2c(-c2ccc3c4c(cccc24)-c2ccccc2O3)c2ccccc12] 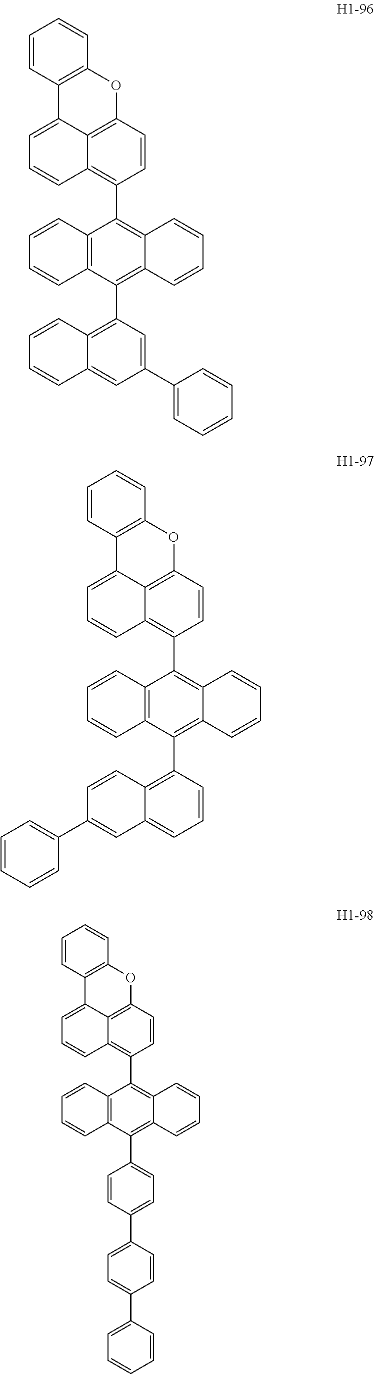 [SMILES: c1ccc(-c2cc(-c3c4ccccc4c(-c4ccc5c6c(cccc46)-c4ccccc4O5)c4ccccc34)c3ccccc3c2)cc1 ; c1ccc(-c2ccc3c(-c4c5ccccc5c(-c5ccc6c7c(cccc57)-c5ccccc5O6)c5ccccc45)cccc3c2)cc1 ; c1ccc(-c2ccc(-c3ccc(-c4c5ccccc5c(-c5ccc6c7c(cccc57)-c5ccccc5O6)c5ccccc45)cc3)cc2)cc1] 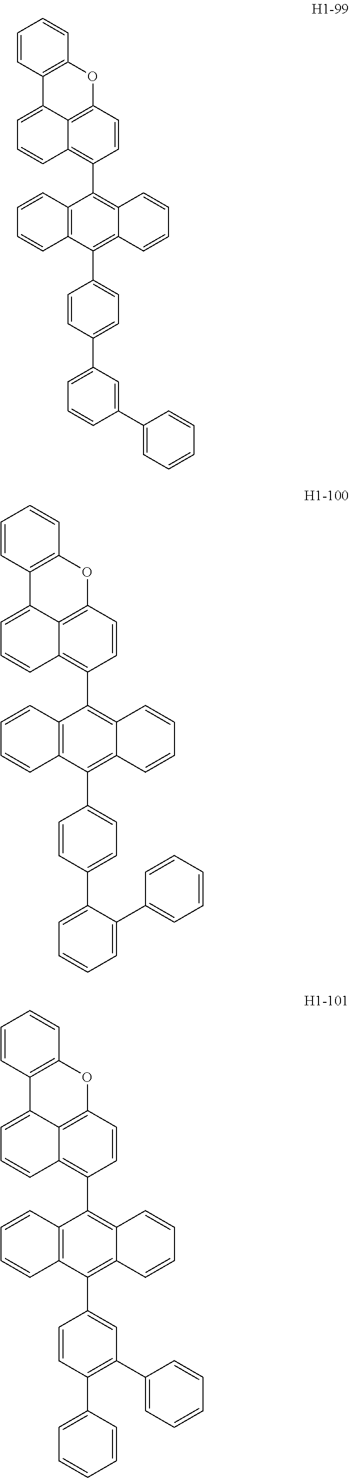 [SMILES: c1ccc(-c2cccc(-c3ccc(-c4c5ccccc5c(-c5ccc6c7c(cccc57)-c5ccccc5O6)c5ccccc45)cc3)c2)cc1 ; c1ccc(-c2ccccc2-c2ccc(-c3c4ccccc4c(-c4ccc5c6c(cccc46)-c4ccccc4O5)c4ccccc34)cc2)cc1 ; c1ccc(-c2ccc(-c3c4ccccc4c(-c4ccc5c6c(cccc46)-c4ccccc4O5)c4ccccc34)cc2-c2ccccc2)cc1] 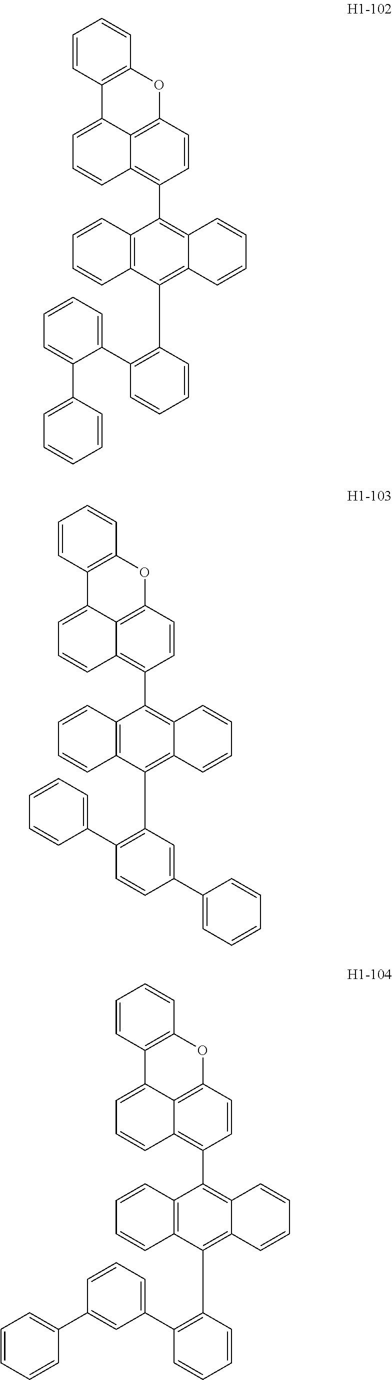 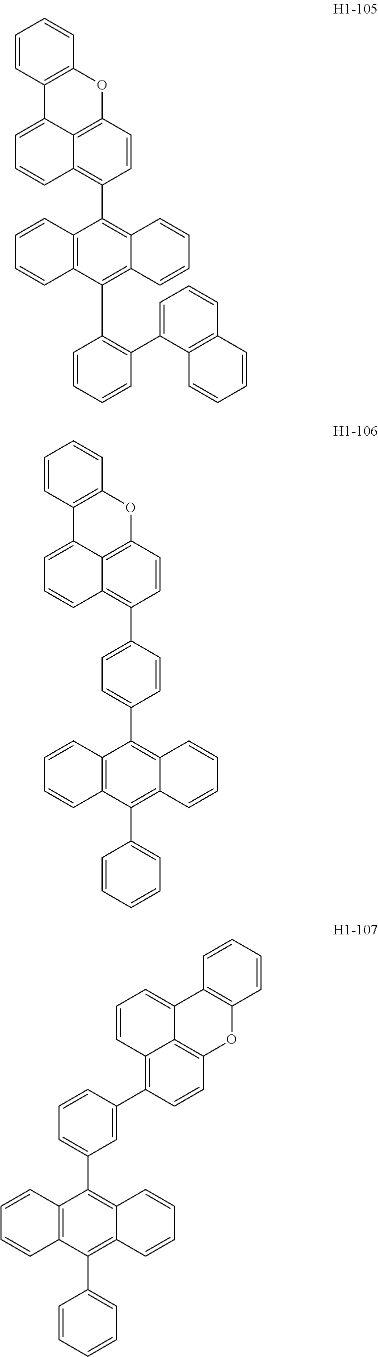 [SMILES: c1ccc2c(c1)Oc1ccc(-c3c4ccccc4c(-c4ccccc4-c4cccc5ccccc45)c4ccccc34)c3cccc-2c13 ; c1ccc(-c2c3ccccc3c(-c3ccc(-c4ccc5c6c(cccc46)-c4ccccc4O5)cc3)c3ccccc23)cc1 ; c1ccc(-c2c3ccccc3c(-c3cccc(-c4ccc5c6c(cccc46)-c4ccccc4O5)c3)c3ccccc23)cc1] 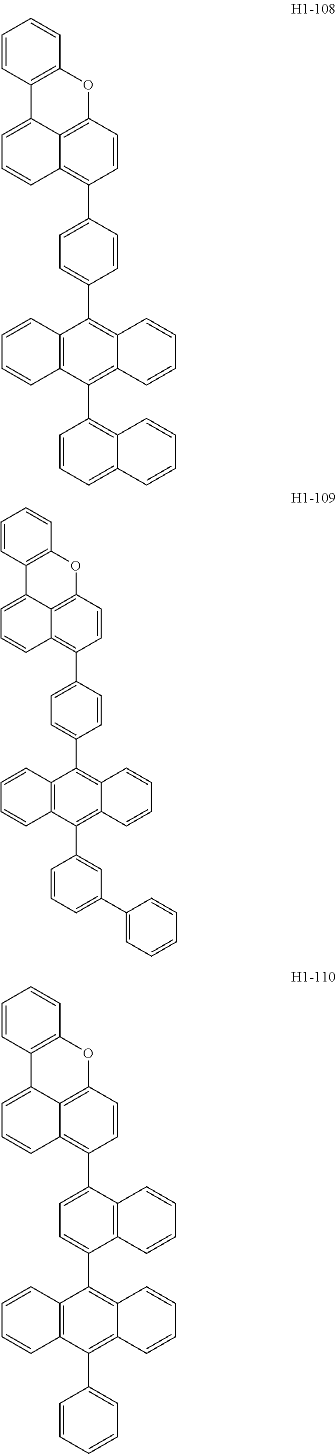 [SMILES: c1ccc2c(c1)Oc1ccc(-c3ccc(-c4c5ccccc5c(-c5cccc6ccccc56)c5ccccc45)cc3)c3cccc-2c13 ; c1ccc(-c2cccc(-c3c4ccccc4c(-c4ccc(-c5ccc6c7c(cccc57)-c5ccccc5O6)cc4)c4ccccc34)c2)cc1 ; c1ccc(-c2c3ccccc3c(-c3ccc(-c4ccc5c6c(cccc46)-c4ccccc4O5)c4ccccc34)c3ccccc23)cc1] 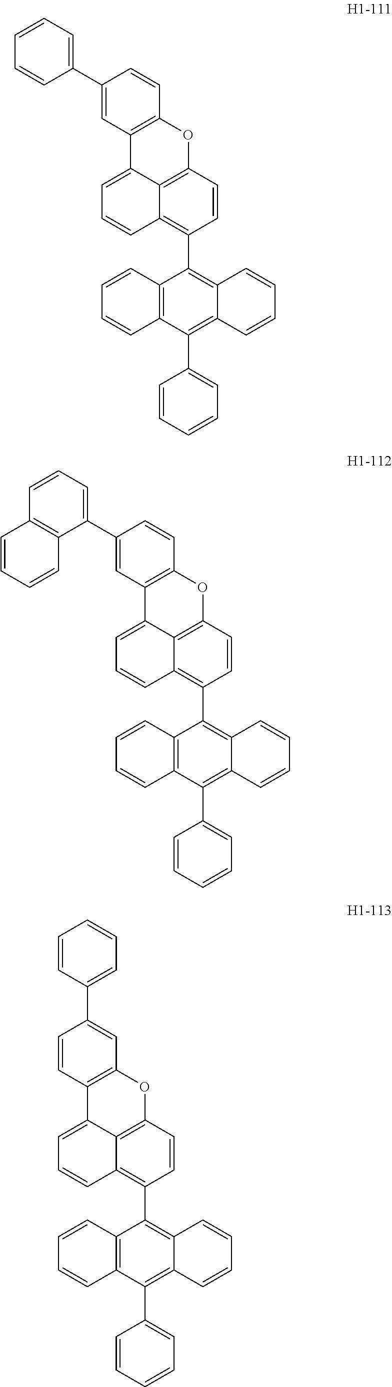 [SMILES: c1ccc(-c2ccc3c(c2)-c2cccc4c(-c5c6ccccc6c(-c6ccccc6)c6ccccc56)ccc(c24)O3)cc1 ; c1ccc(-c2c3ccccc3c(-c3ccc4c5c(cccc35)-c3cc(-c5cccc6ccccc56)ccc3O4)c3ccccc23)cc1 ; c1ccc(-c2ccc3c(c2)Oc2ccc(-c4c5ccccc5c(-c5ccccc5)c5ccccc45)c4cccc-3c24)cc1] 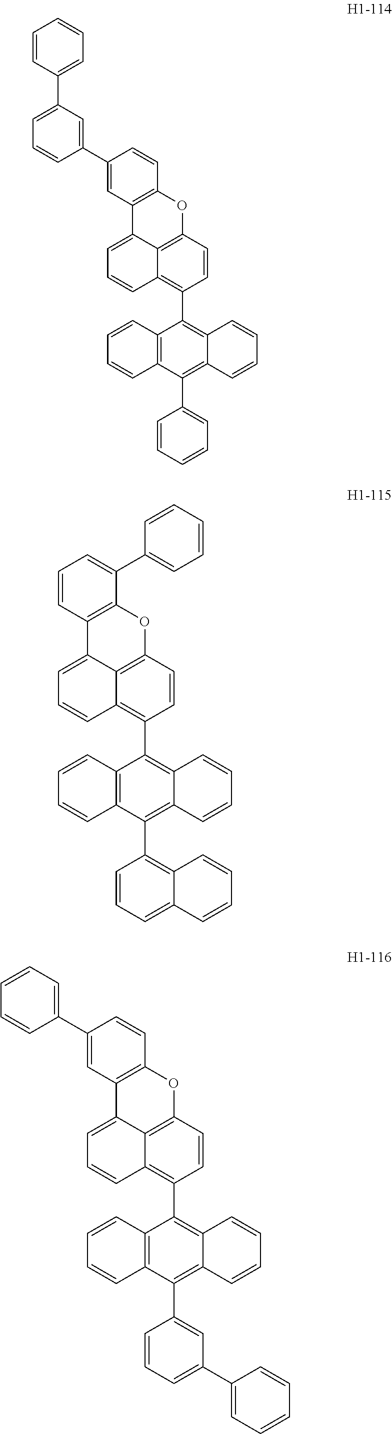 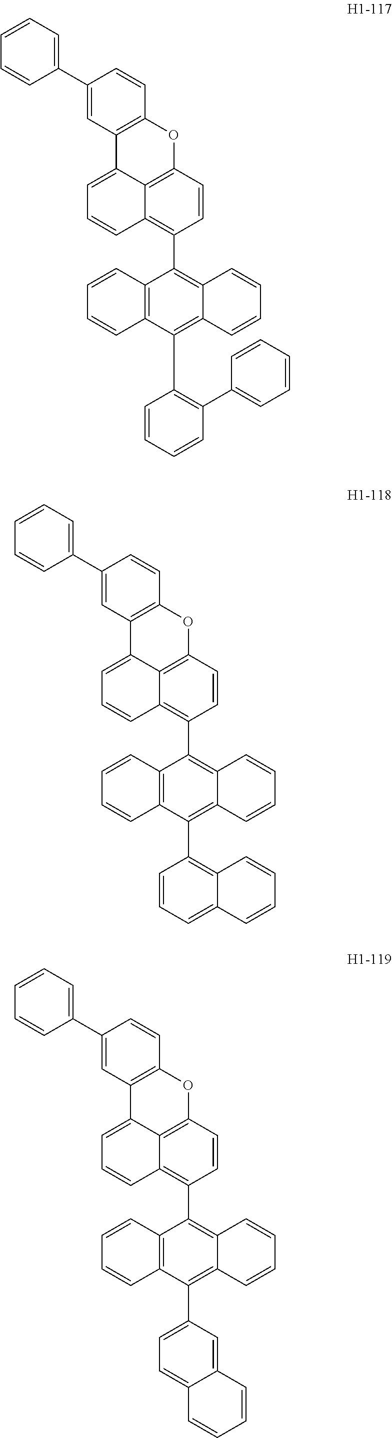 [SMILES: c1ccc(-c2ccc3c(c2)-c2cccc4c(-c5c6ccccc6c(-c6ccccc6-c6ccccc6)c6ccccc56)ccc(c24)O3)cc1 ; c1ccc(-c2ccc3c(c2)-c2cccc4c(-c5c6ccccc6c(-c6cccc7ccccc67)c6ccccc56)ccc(c24)O3)cc1 ; c1ccc(-c2ccc3c(c2)-c2cccc4c(-c5c6ccccc6c(-c6ccc7ccccc7c6)c6ccccc56)ccc(c24)O3)cc1] 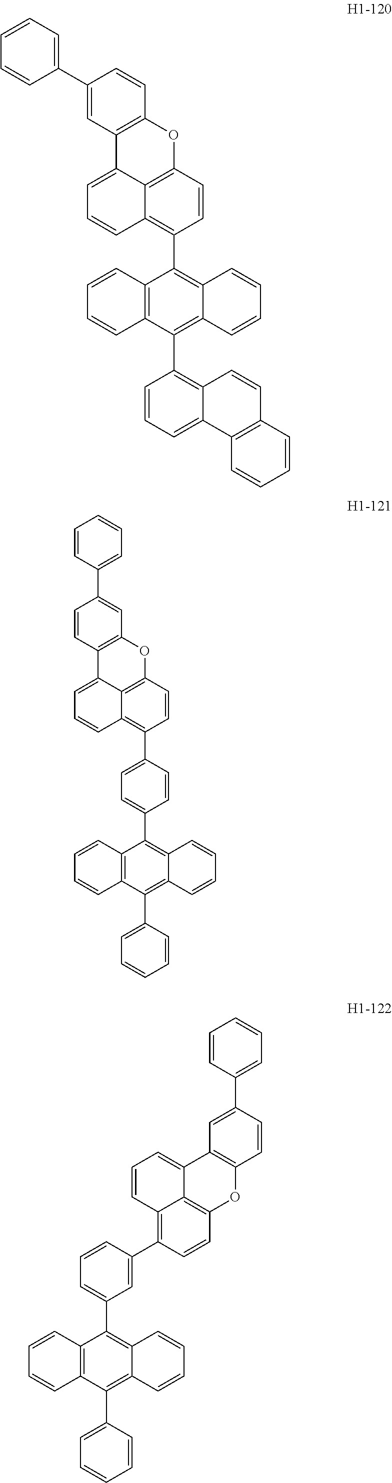 [SMILES: c1ccc(-c2ccc3c(c2)-c2cccc4c(-c5c6ccccc6c(-c6cccc7c6ccc6ccccc67)c6ccccc56)ccc(c24)O3)cc1 ; c1ccc(-c2ccc3c(c2)Oc2ccc(-c4ccc(-c5c6ccccc6c(-c6ccccc6)c6ccccc56)cc4)c4cccc-3c24)cc1 ; c1ccc(-c2ccc3c(c2)-c2cccc4c(-c5cccc(-c6c7ccccc7c(-c7ccccc7)c7ccccc67)c5)ccc(c24)O3)cc1] 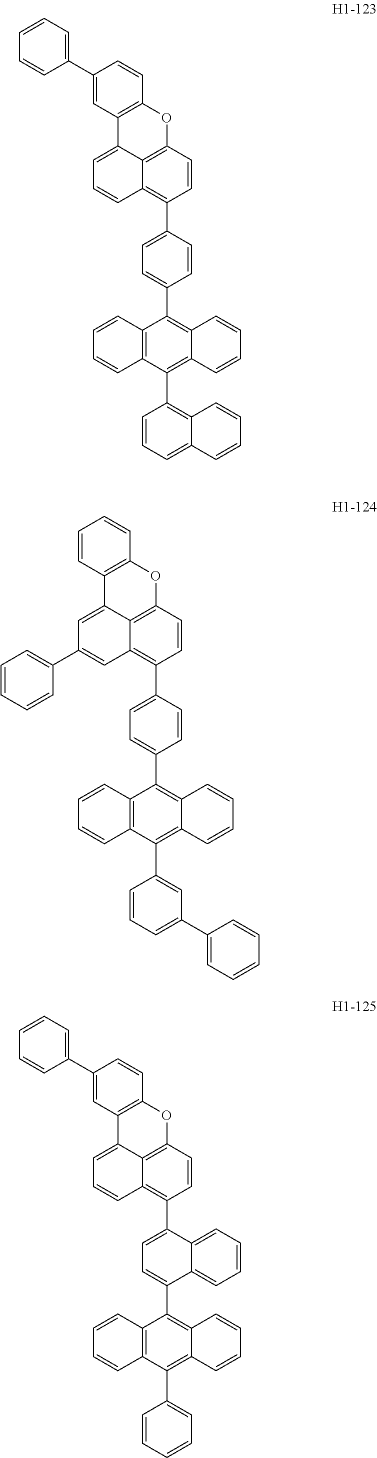 [SMILES: c1ccc(-c2ccc3c(c2)-c2cccc4c(-c5ccc(-c6c7ccccc7c(-c7cccc8ccccc78)c7ccccc67)cc5)ccc(c24)O3)cc1 ; c1ccc(-c2cccc(-c3c4ccccc4c(-c4ccc(-c5ccc6c7c(cc(-c8ccccc8)cc57)-c5ccccc5O6)cc4)c4ccccc34)c2)cc1 ; c1ccc(-c2ccc3c(c2)-c2cccc4c(-c5ccc(-c6c7ccccc7c(-c7ccccc7)c7ccccc67)c6ccccc56)ccc(c24)O3)cc1] 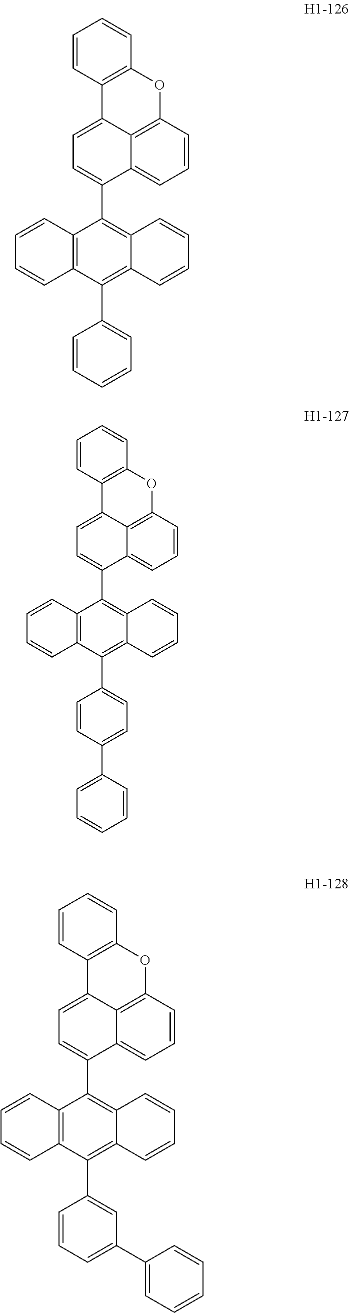 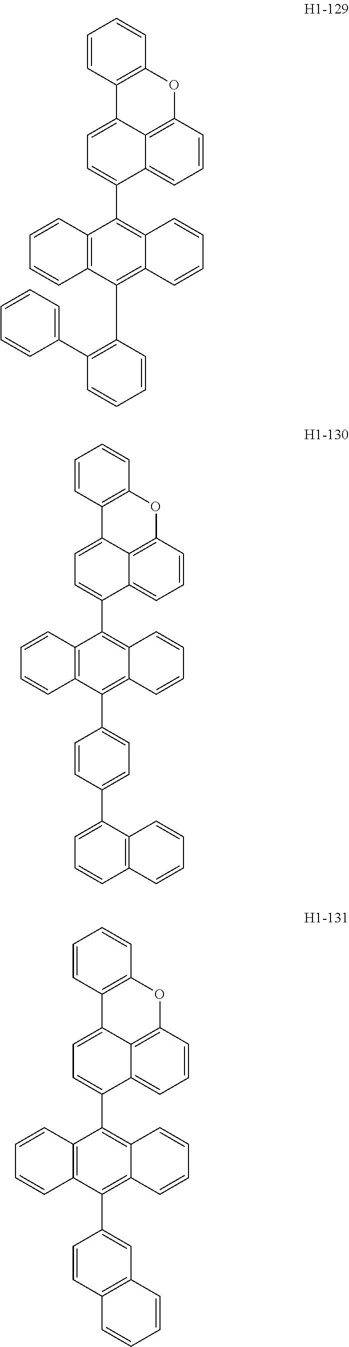 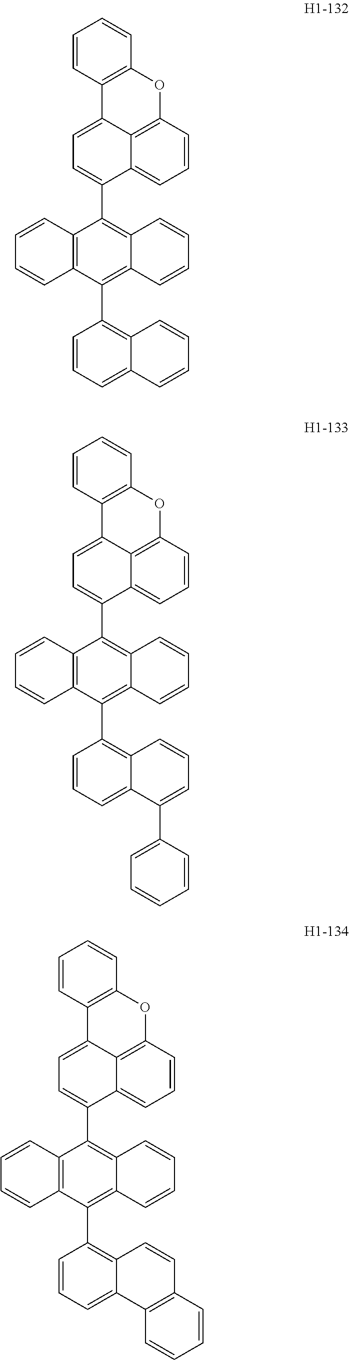 [SMILES: c1ccc2c(c1)Oc1cccc3c(-c4c5ccccc5c(-c5cccc6ccccc56)c5ccccc45)ccc-2c13 ; c1ccc(-c2cccc3c(-c4c5ccccc5c(-c5ccc6c7c(cccc57)Oc5ccccc5-6)c5ccccc45)cccc23)cc1 ; c1ccc2c(c1)Oc1cccc3c(-c4c5ccccc5c(-c5cccc6c5ccc5ccccc56)c5ccccc45)ccc-2c13] 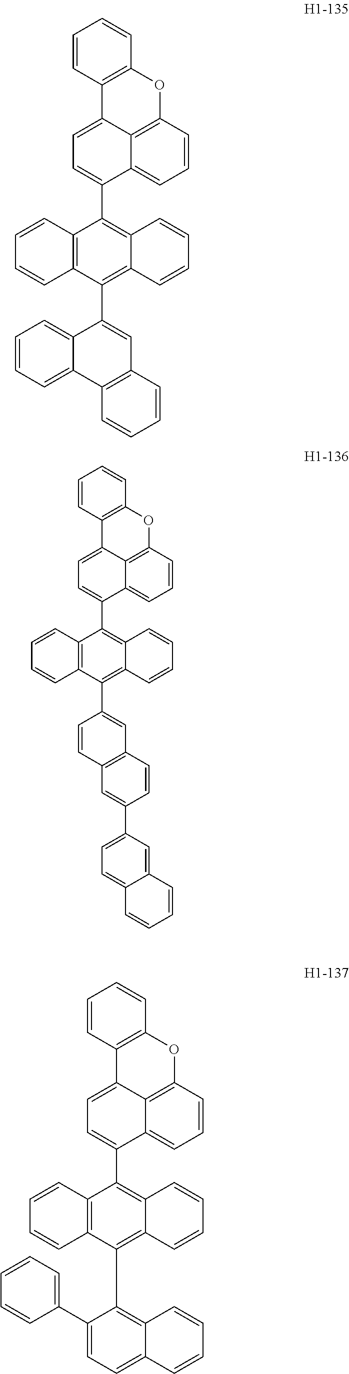 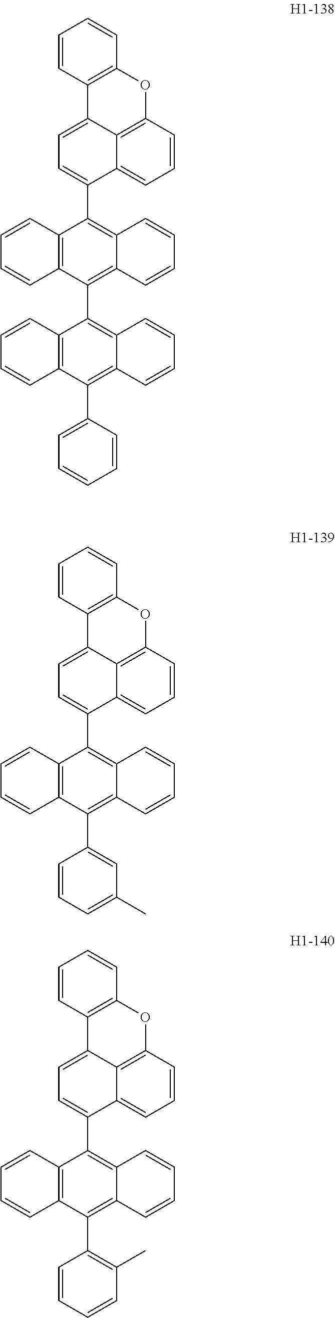 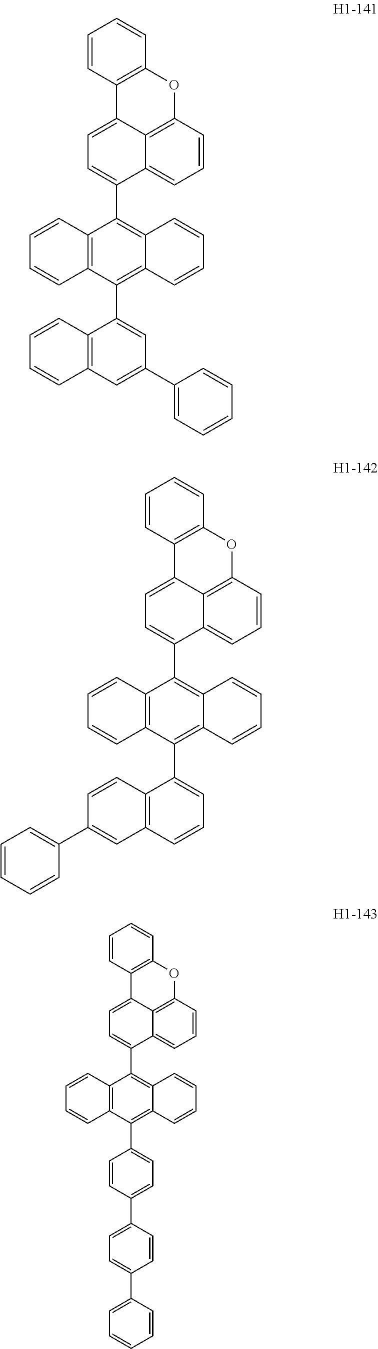 [SMILES: c1ccc(-c2cc(-c3c4ccccc4c(-c4ccc5c6c(cccc46)Oc4ccccc4-5)c4ccccc34)c3ccccc3c2)cc1 ; c1ccc(-c2ccc3c(-c4c5ccccc5c(-c5ccc6c7c(cccc57)Oc5ccccc5-6)c5ccccc45)cccc3c2)cc1 ; c1ccc(-c2ccc(-c3ccc(-c4c5ccccc5c(-c5ccc6c7c(cccc57)Oc5ccccc5-6)c5ccccc45)cc3)cc2)cc1] 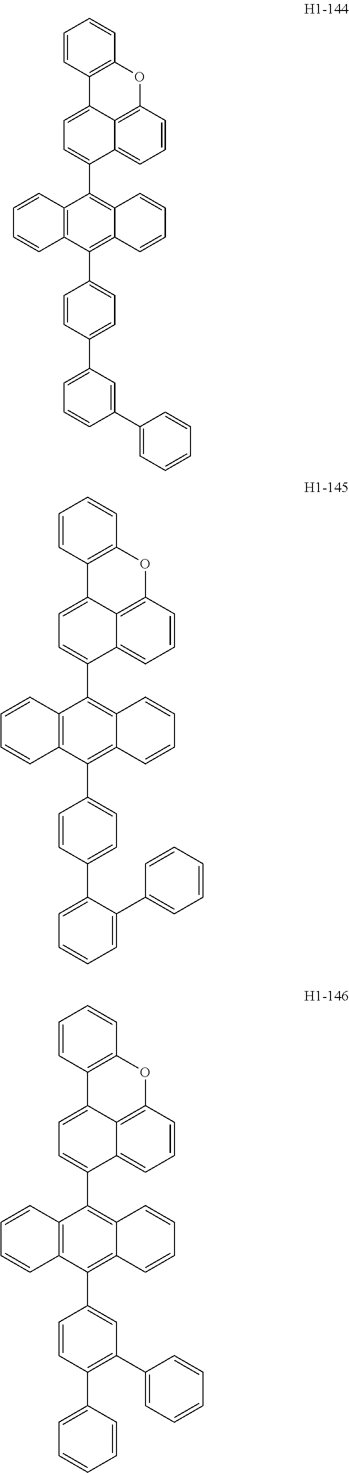 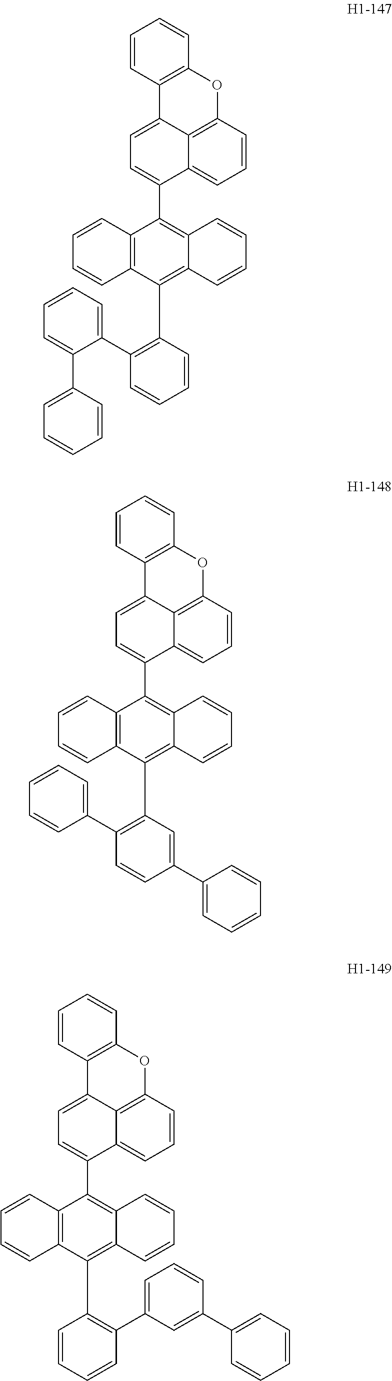 [SMILES: c1ccc(-c2ccccc2-c2ccccc2-c2c3ccccc3c(-c3ccc4c5c(cccc35)Oc3ccccc3-4)c3ccccc23)cc1 ; c1ccc(-c2ccc(-c3ccccc3)c(-c3c4ccccc4c(-c4ccc5c6c(cccc46)Oc4ccccc4-5)c4ccccc34)c2)cc1 ; c1ccc(-c2cccc(-c3ccccc3-c3c4ccccc4c(-c4ccc5c6c(cccc46)Oc4ccccc4-5)c4ccccc34)c2)cc1] 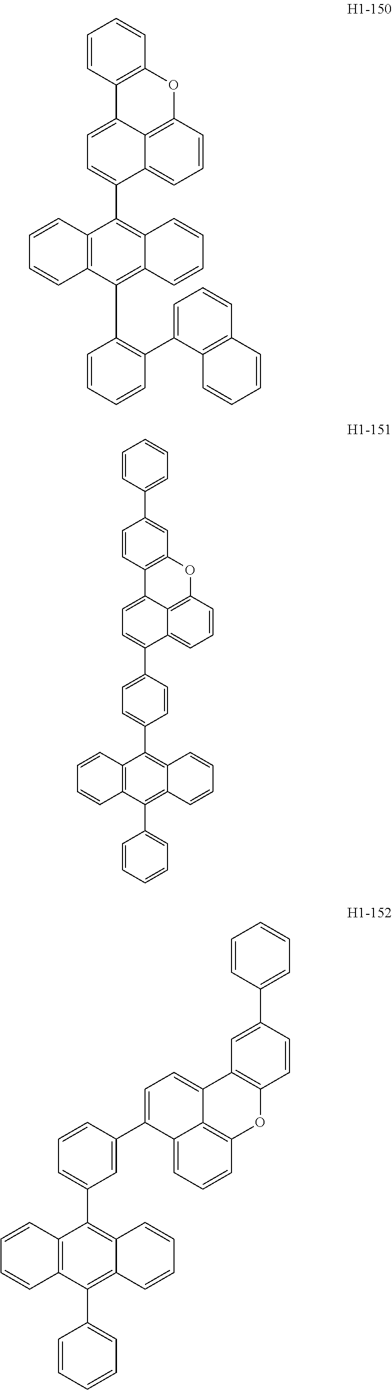 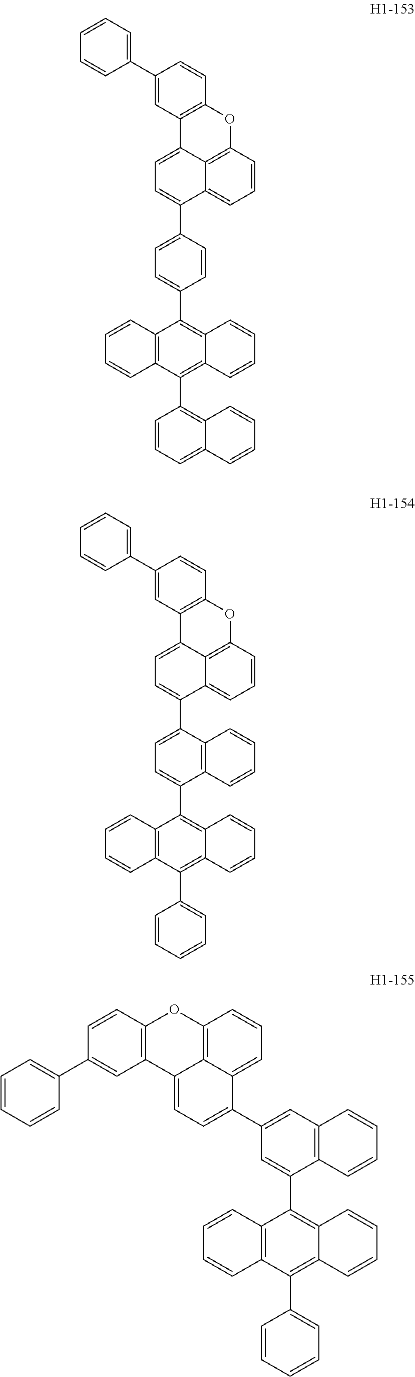 [SMILES: c1ccc(-c2ccc3c(c2)-c2ccc(-c4ccc(-c5c6ccccc6c(-c6cccc7ccccc67)c6ccccc56)cc4)c4cccc(c24)O3)cc1 ; c1ccc(-c2ccc3c(c2)-c2ccc(-c4ccc(-c5c6ccccc6c(-c6ccccc6)c6ccccc56)c5ccccc45)c4cccc(c24)O3)cc1 ; c1ccc(-c2ccc3c(c2)-c2ccc(-c4cc(-c5c6ccccc6c(-c6ccccc6)c6ccccc56)c5ccccc5c4)c4cccc(c24)O3)cc1] 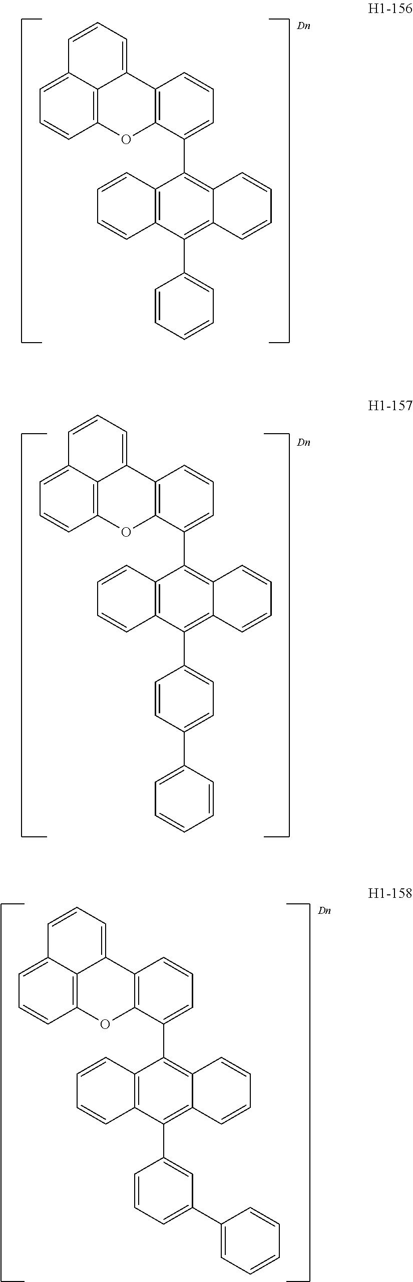 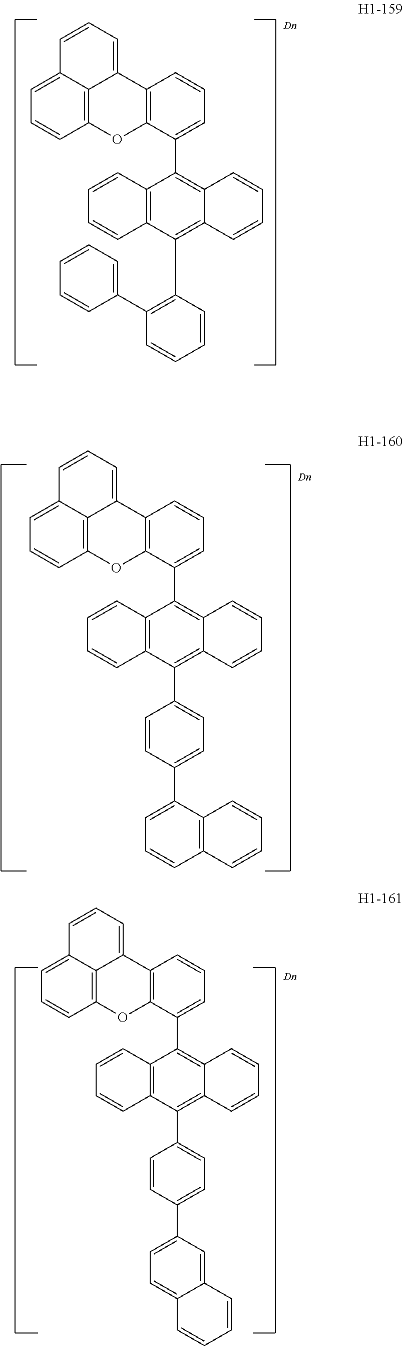 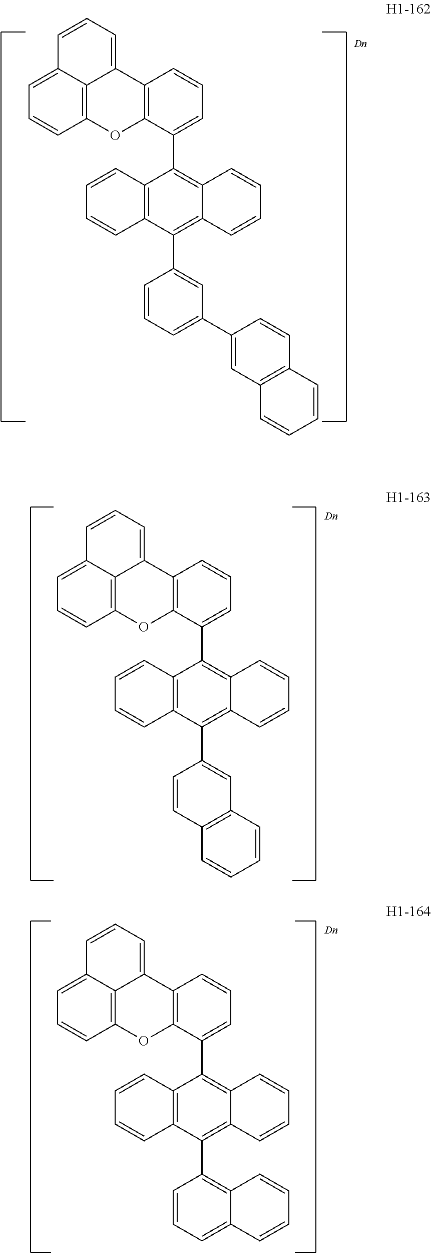 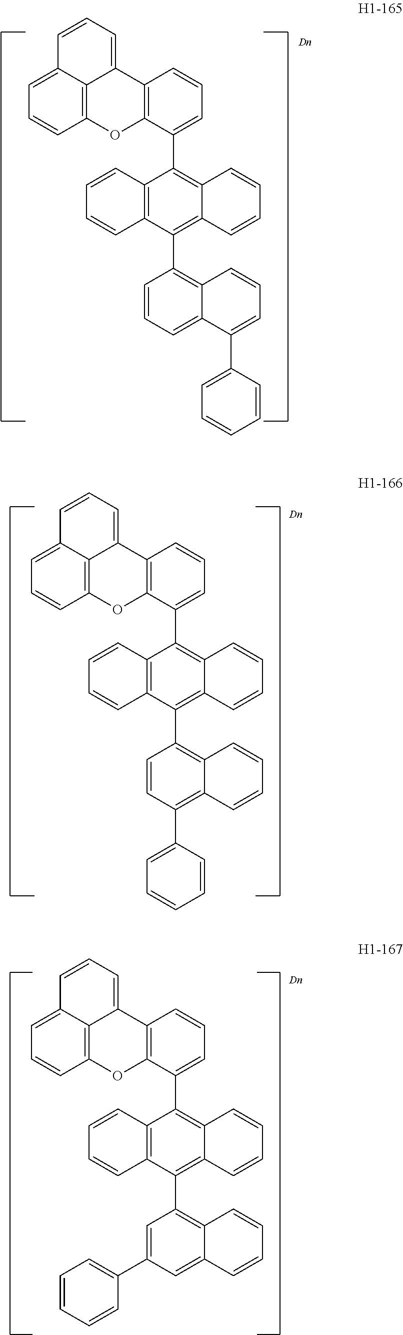 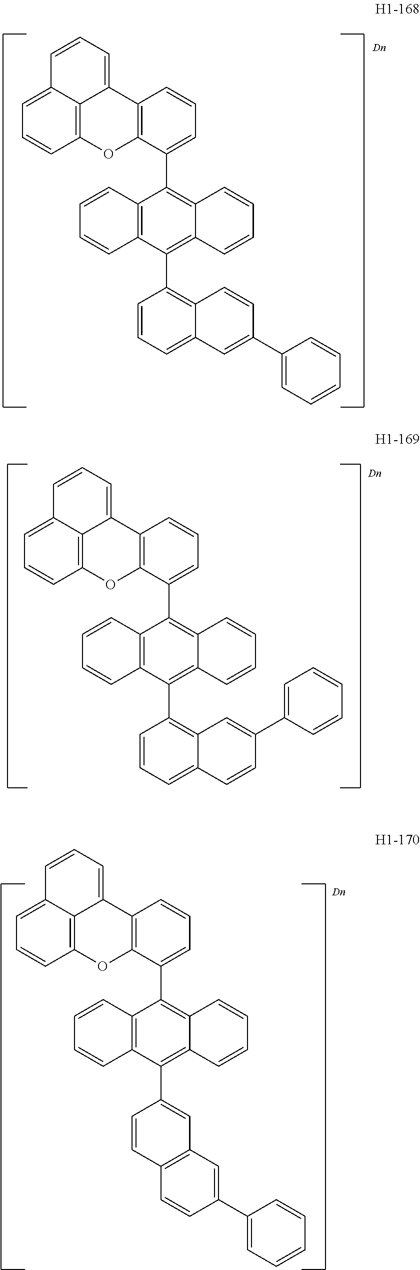 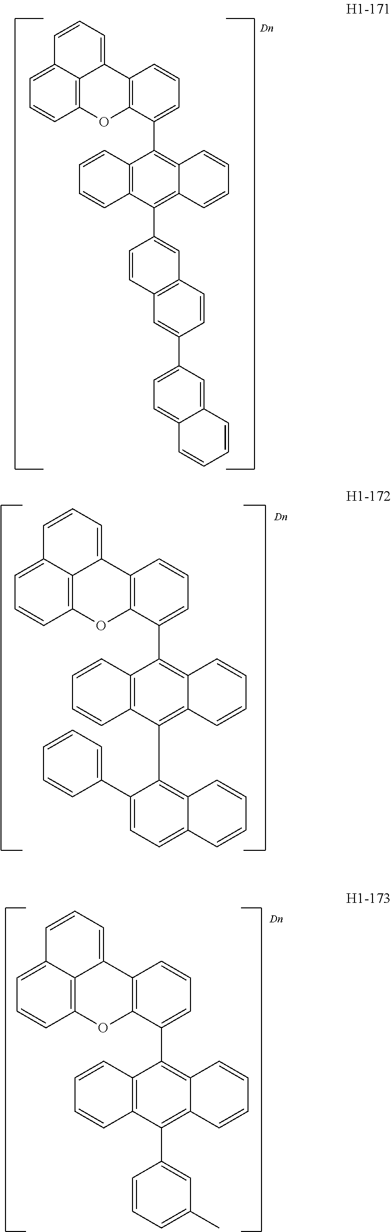 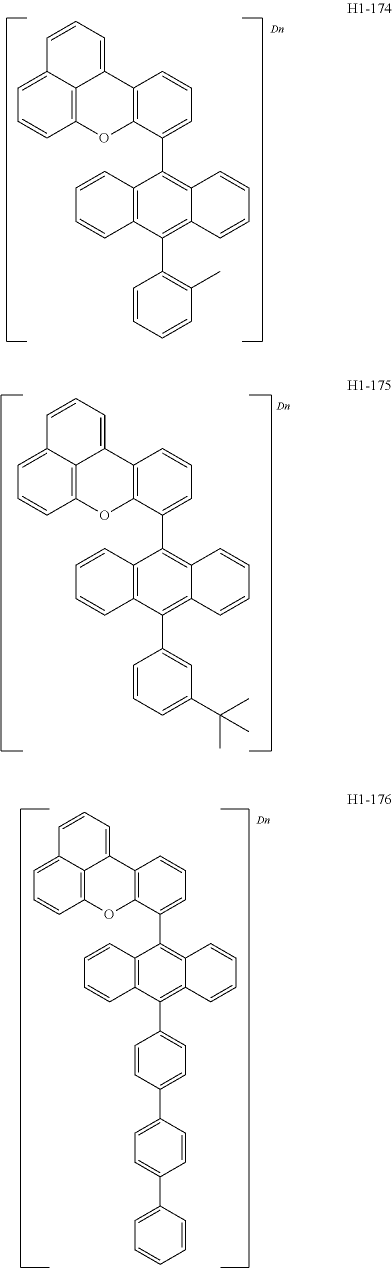 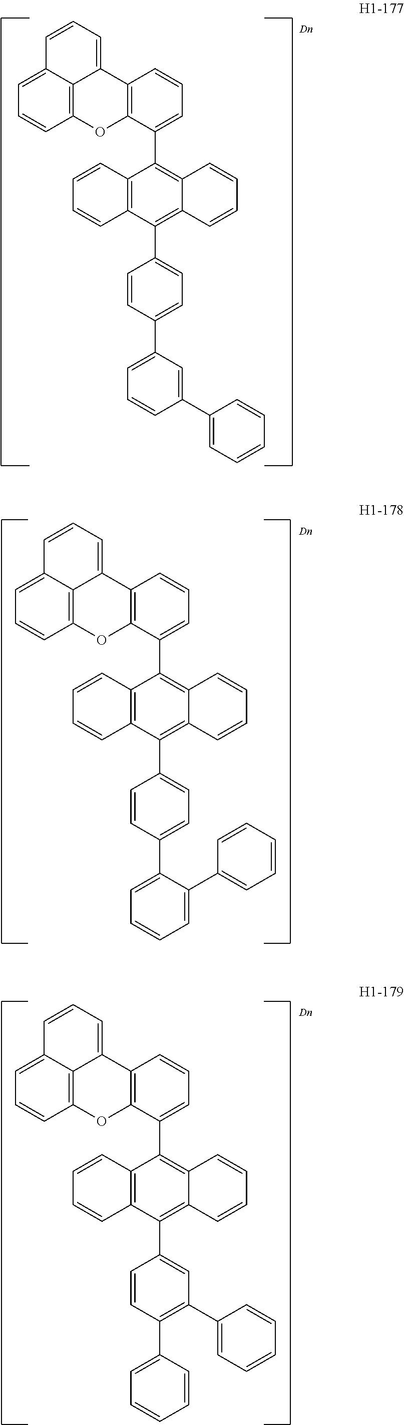 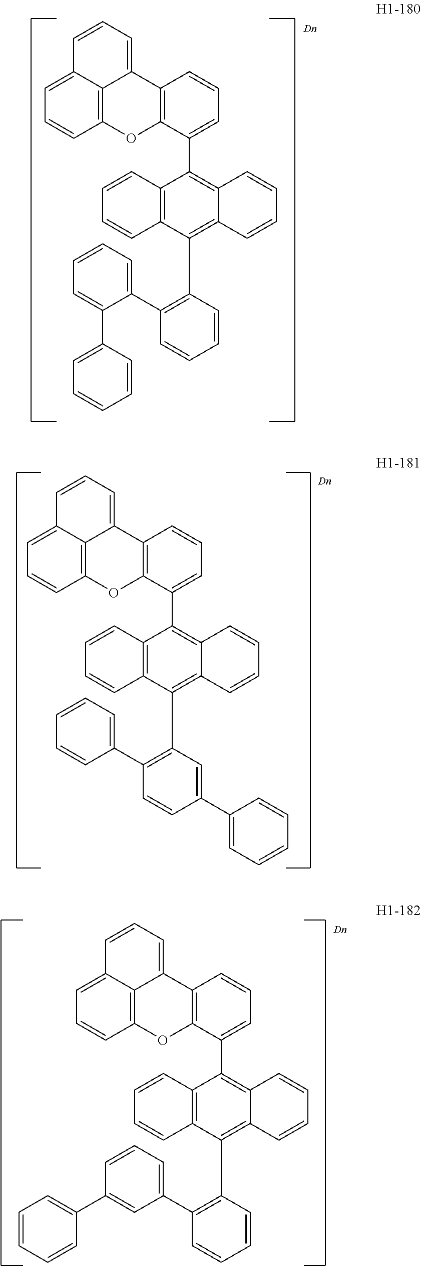 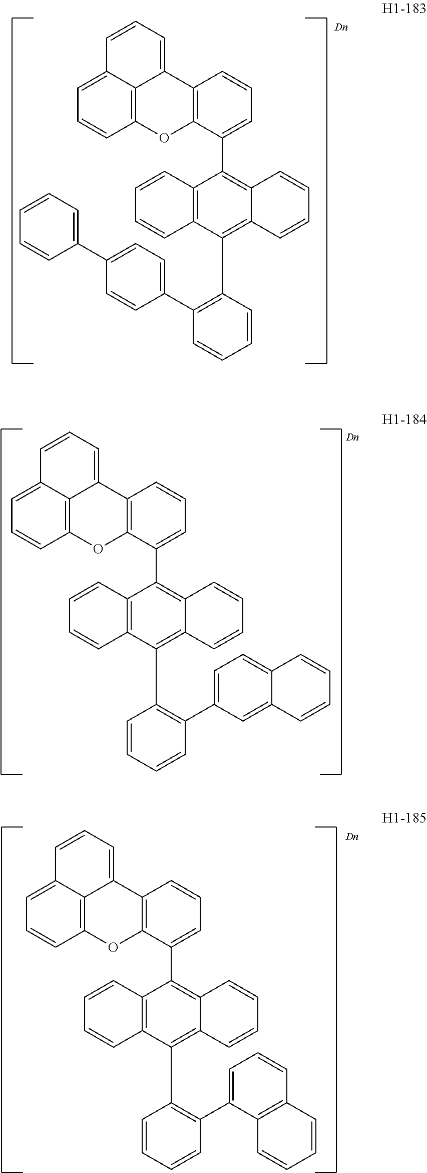 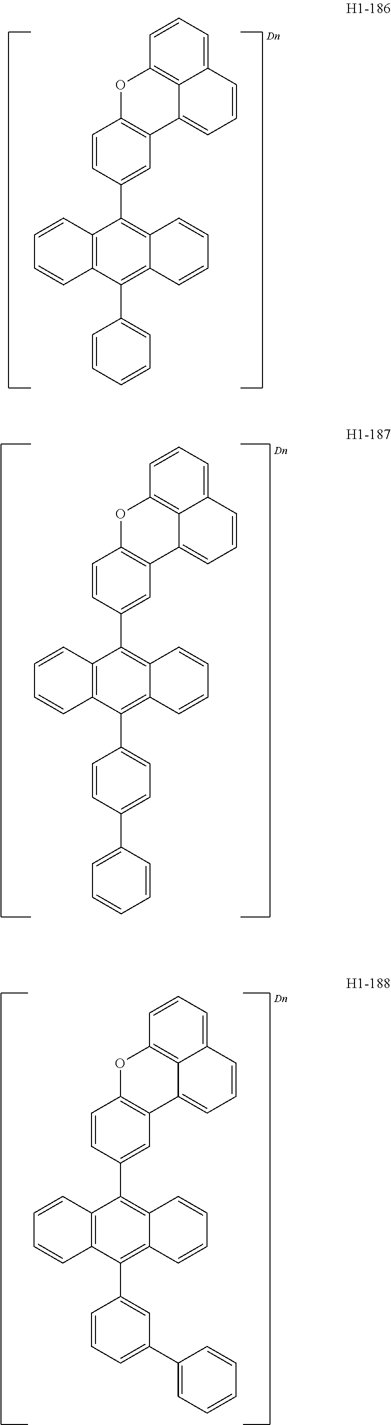 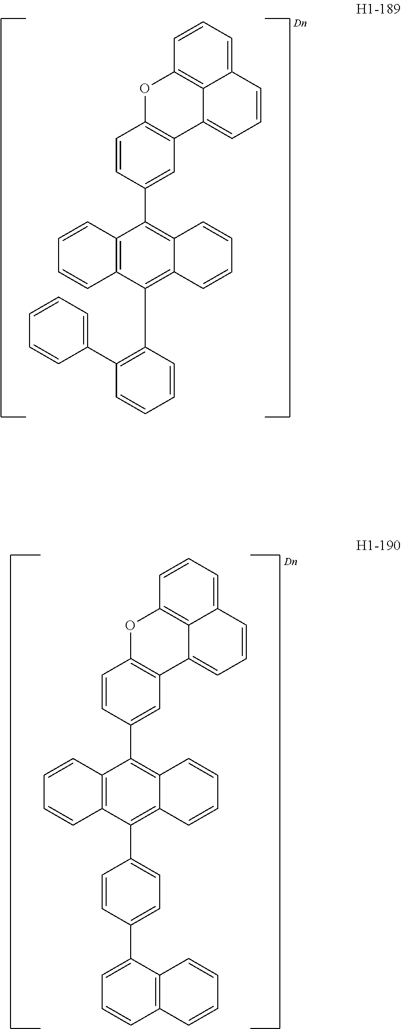 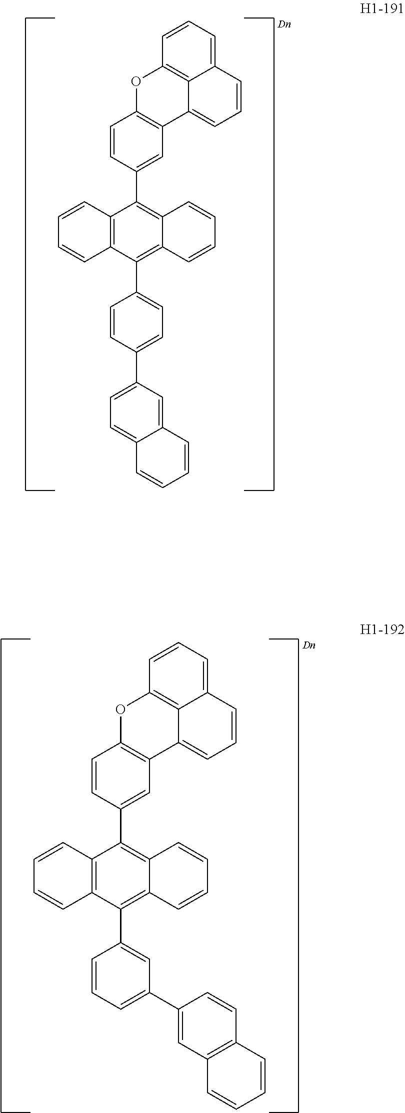 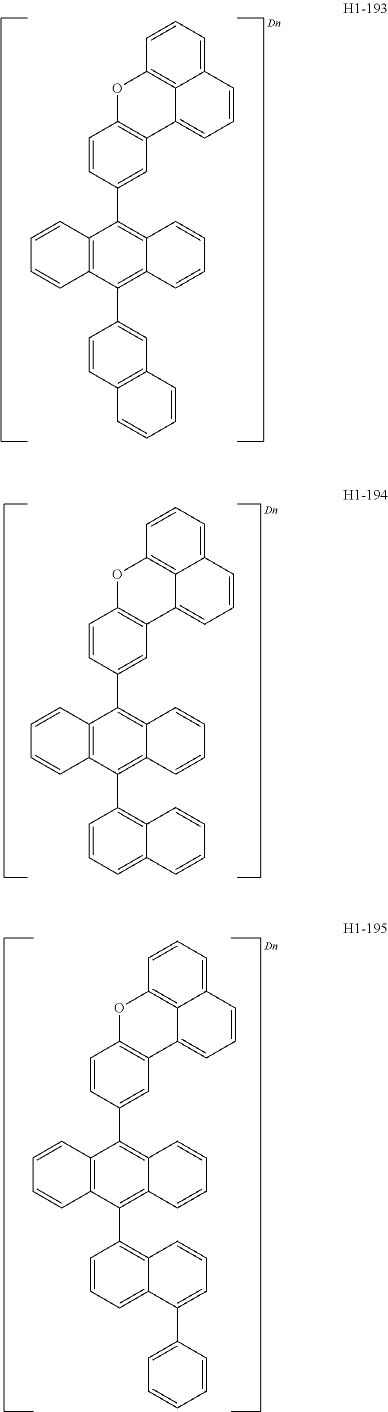 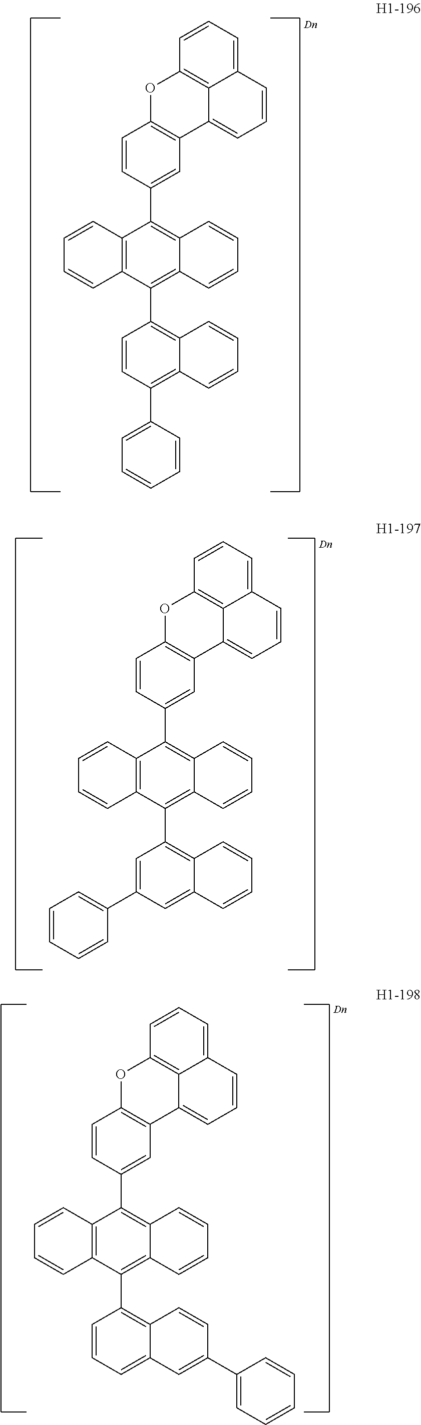 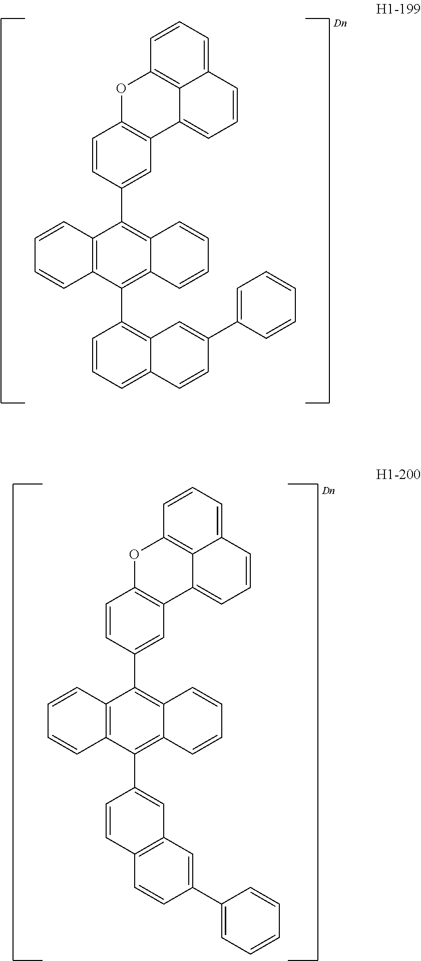 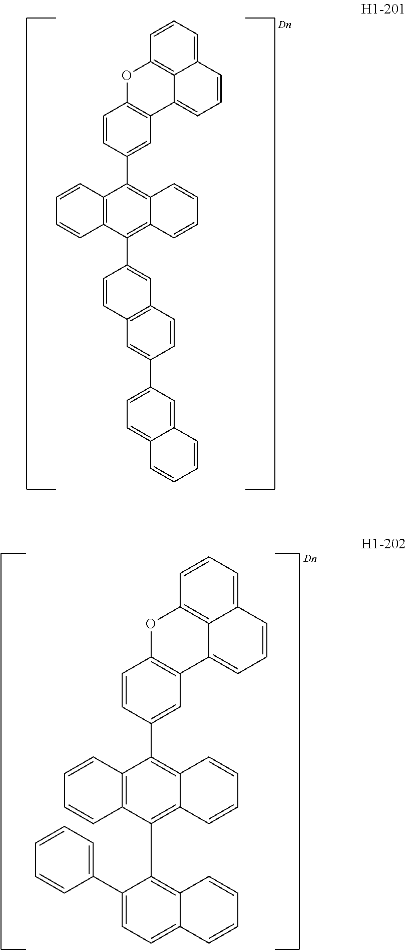 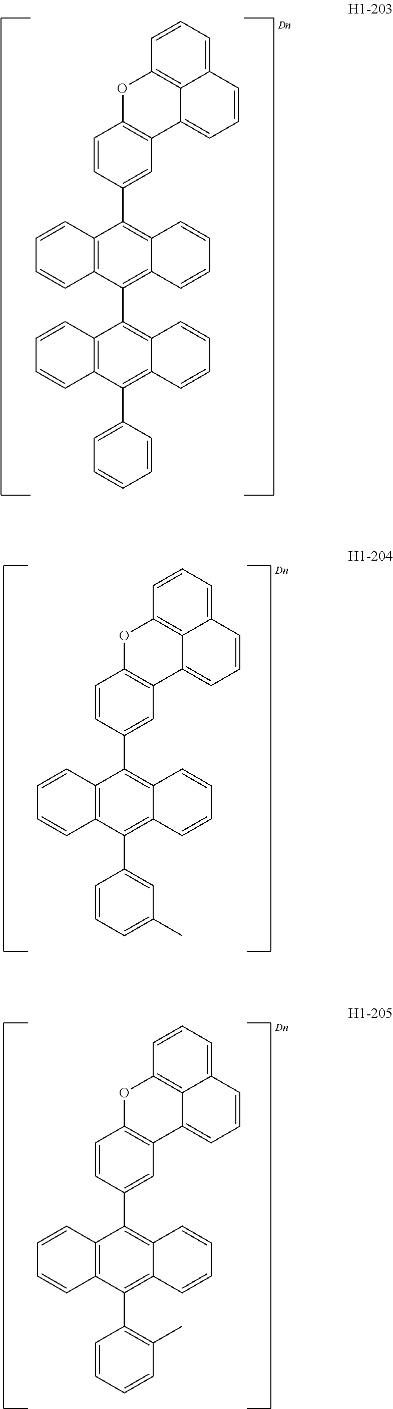 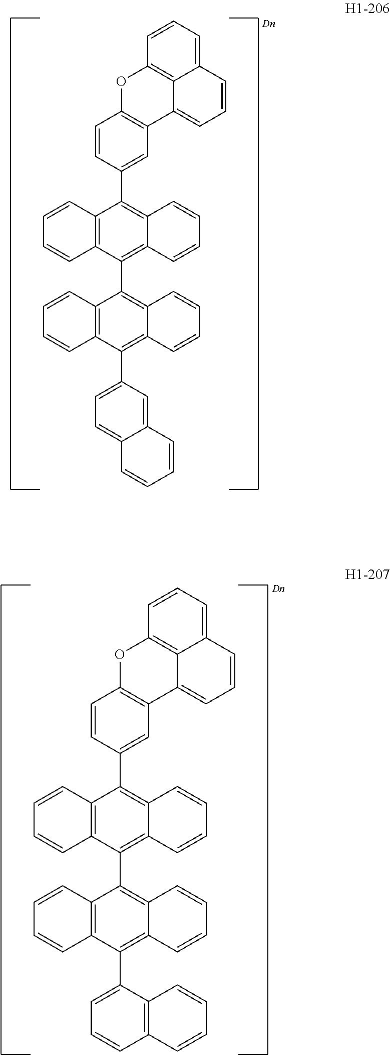 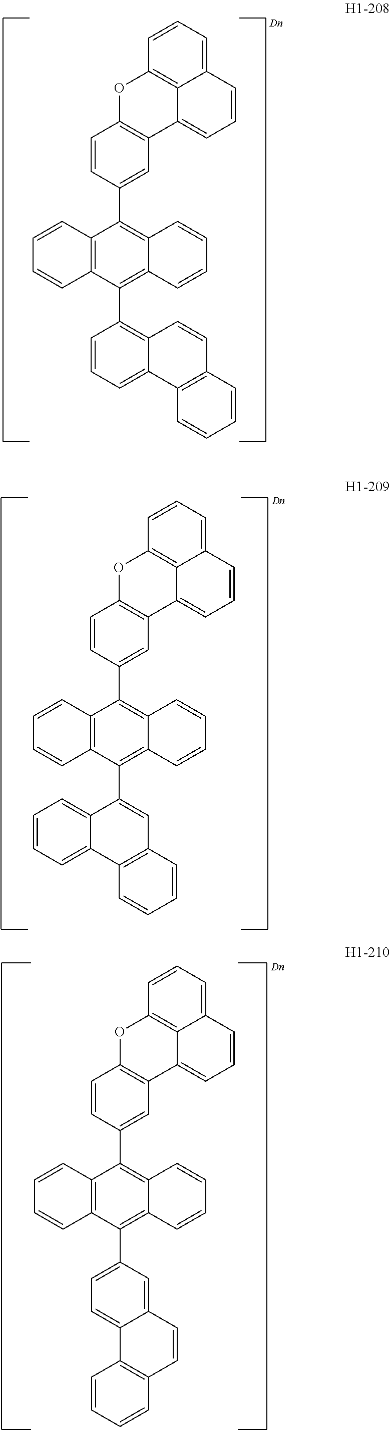 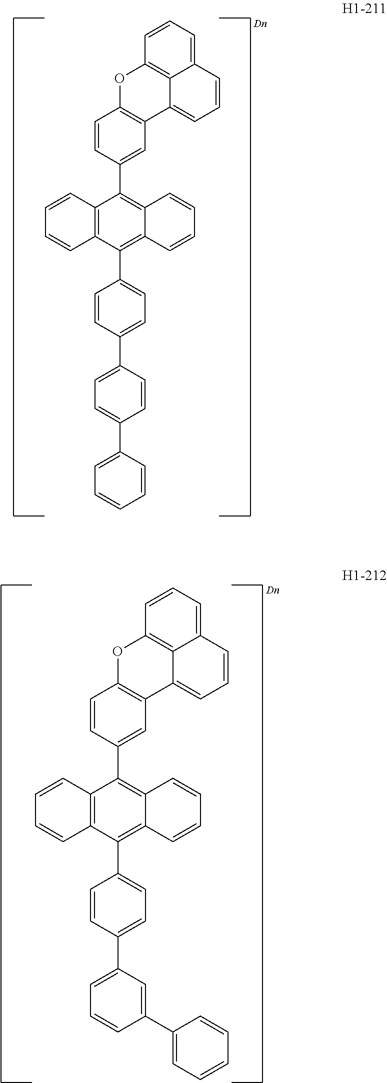 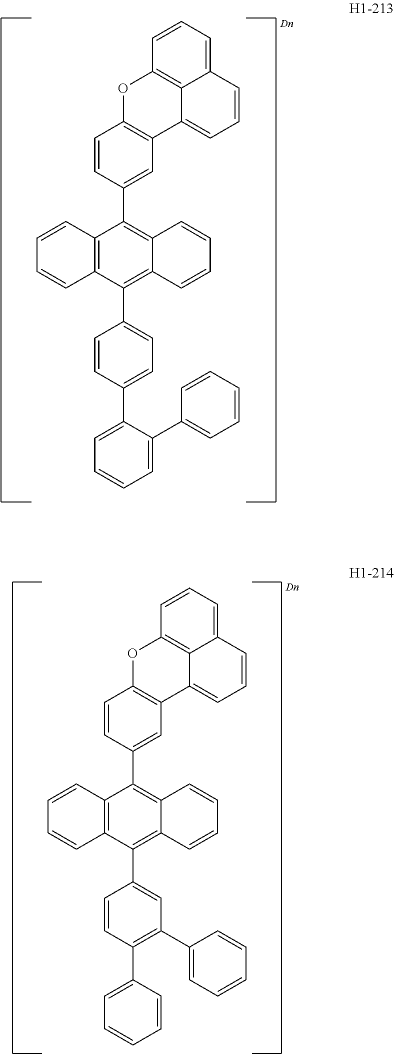 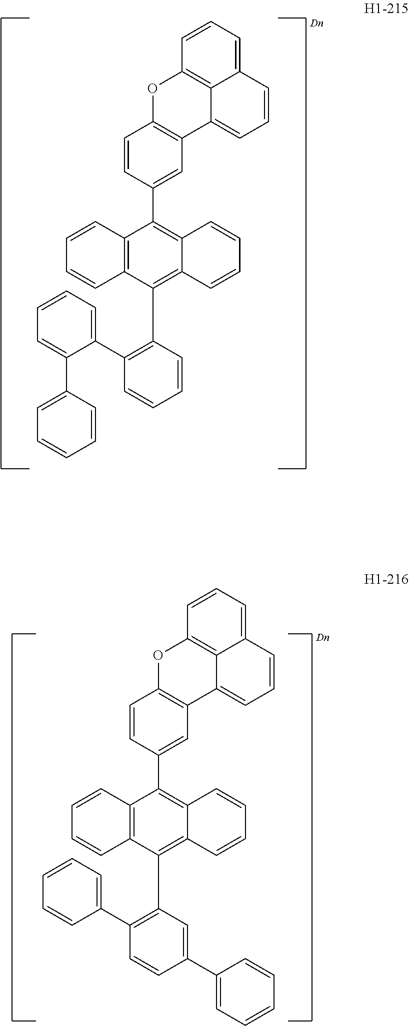 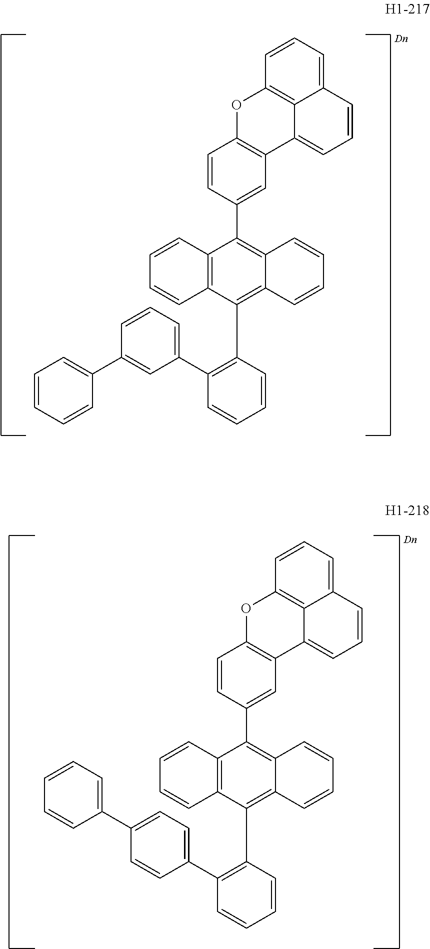 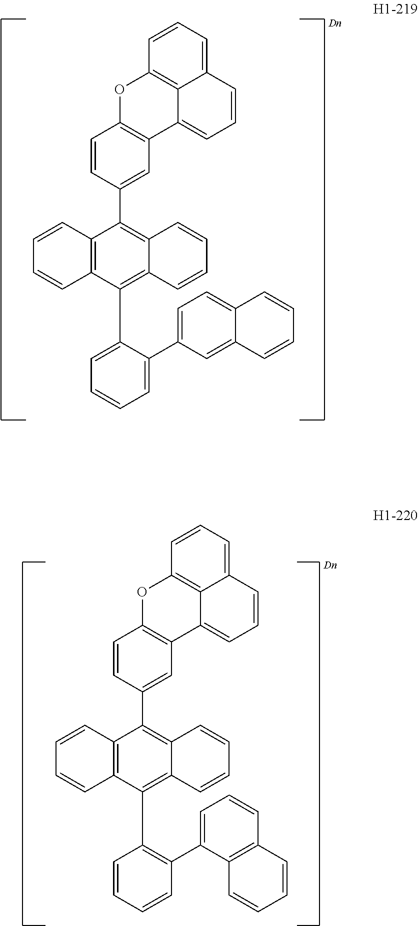 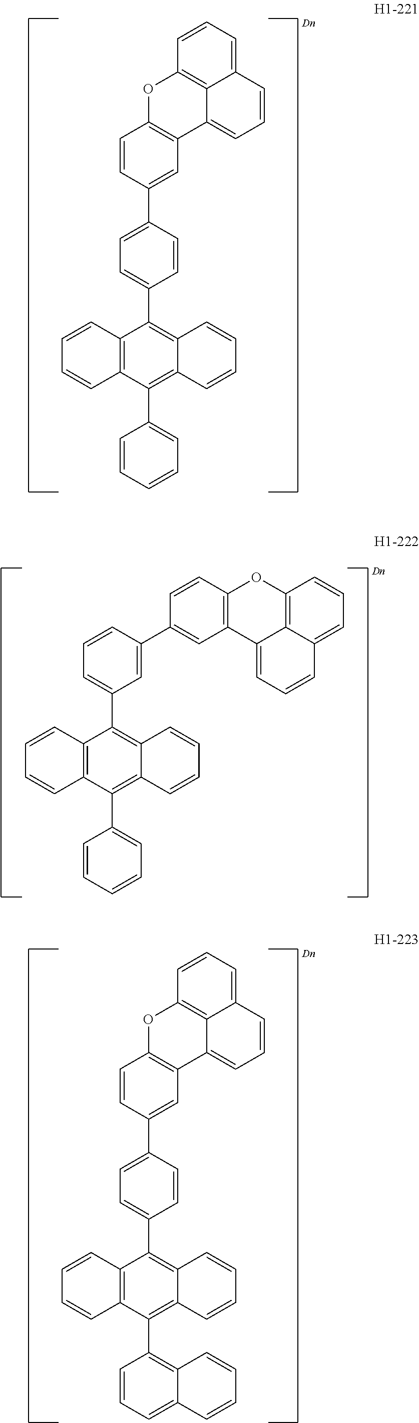 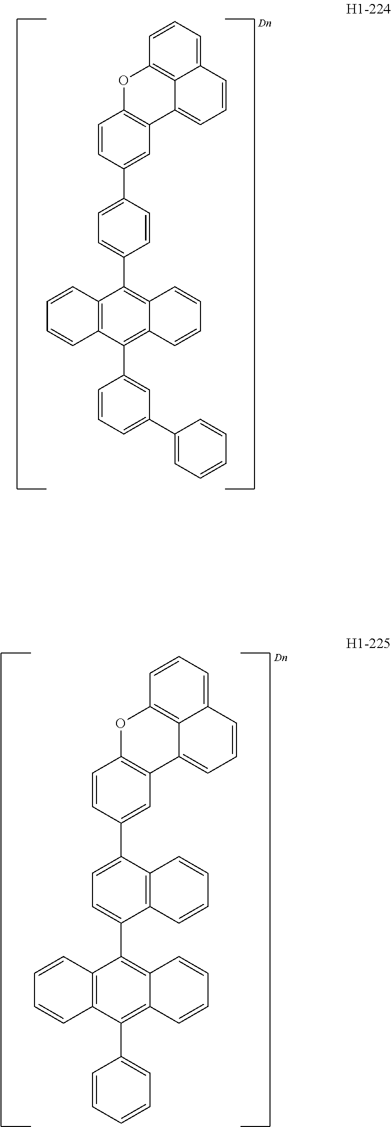 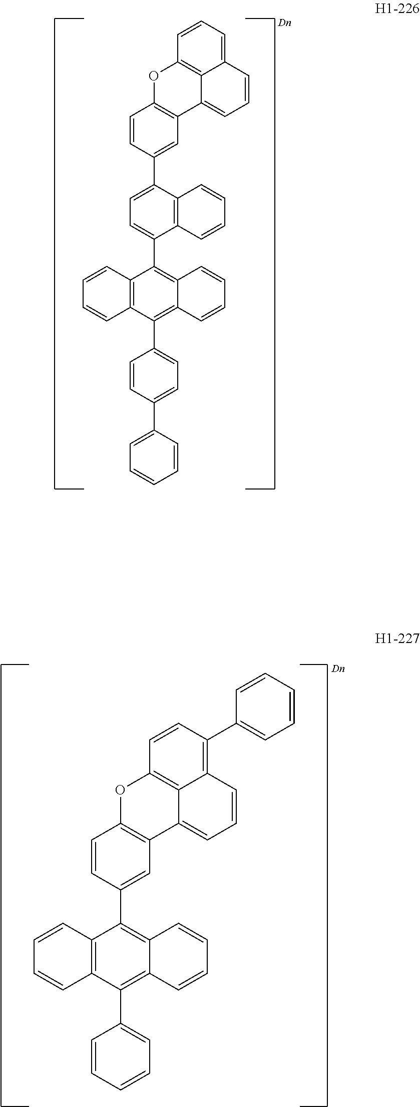 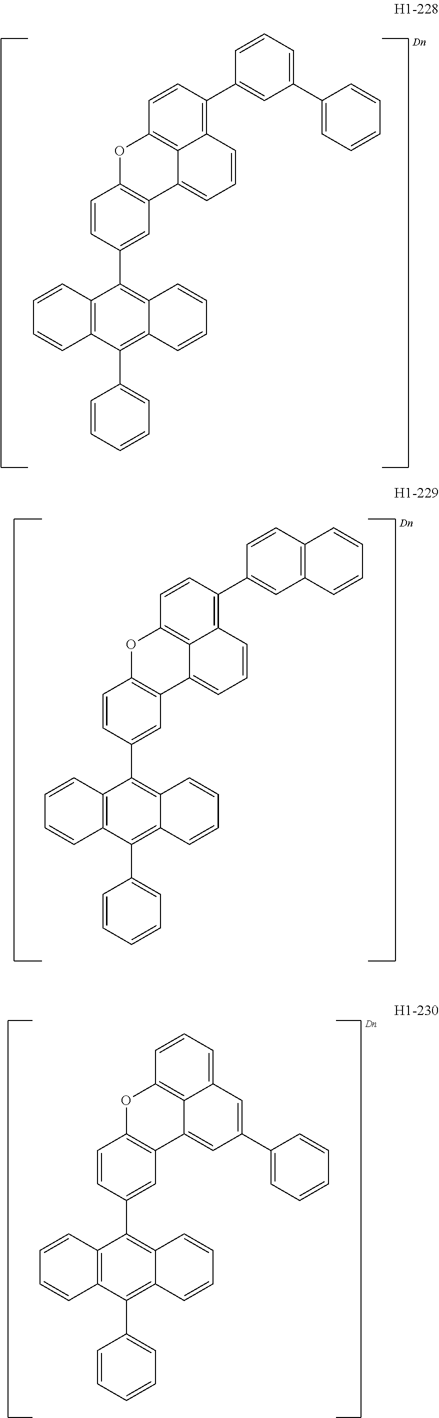 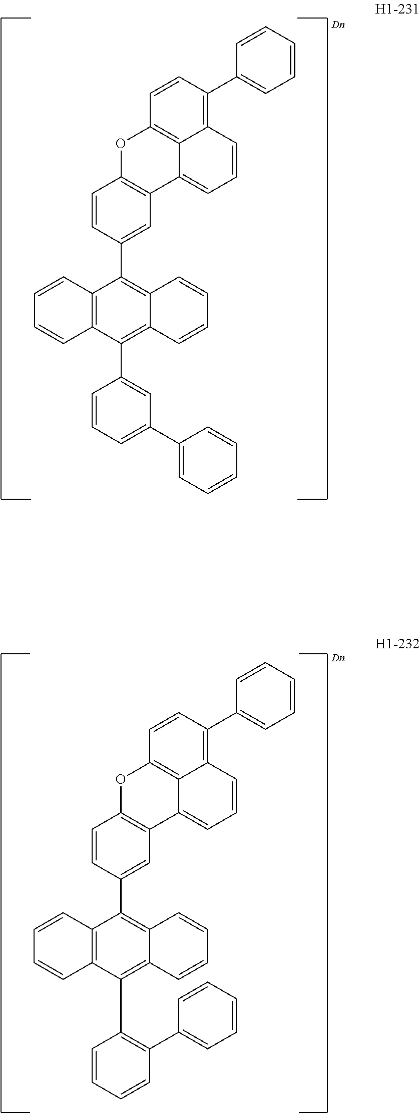 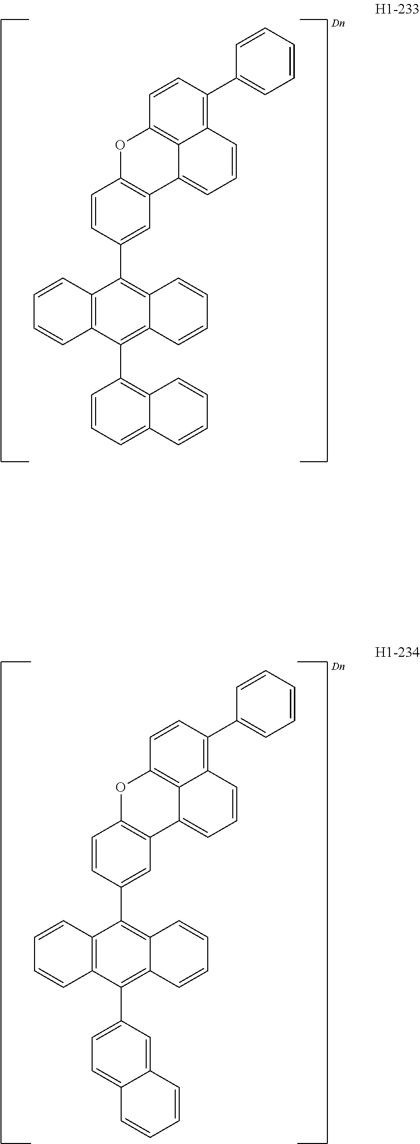 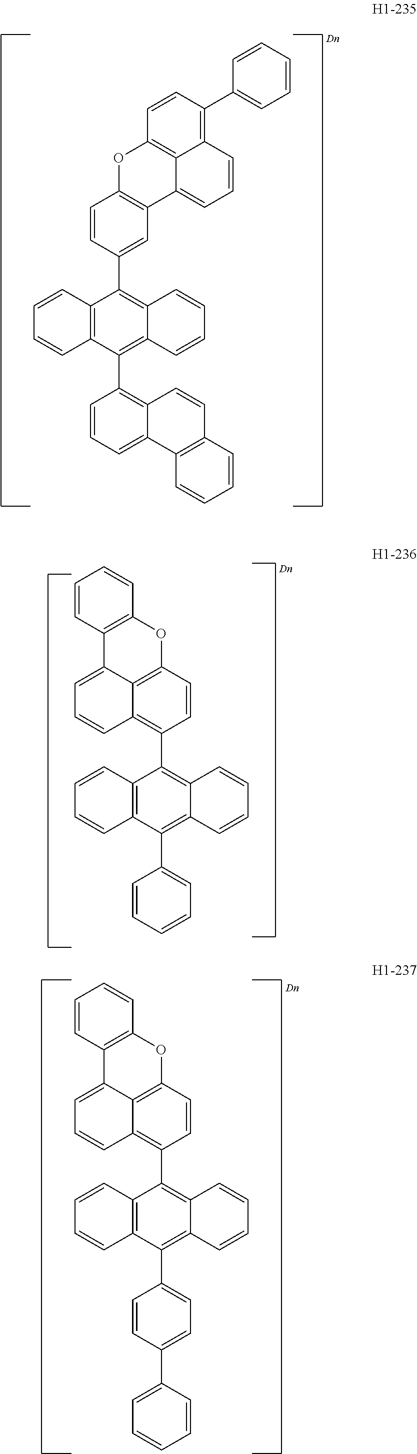 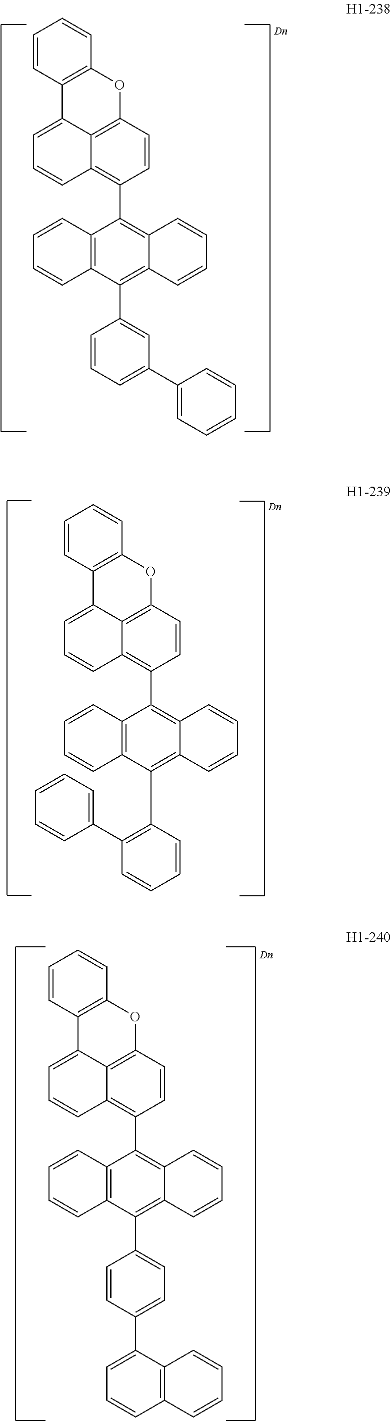 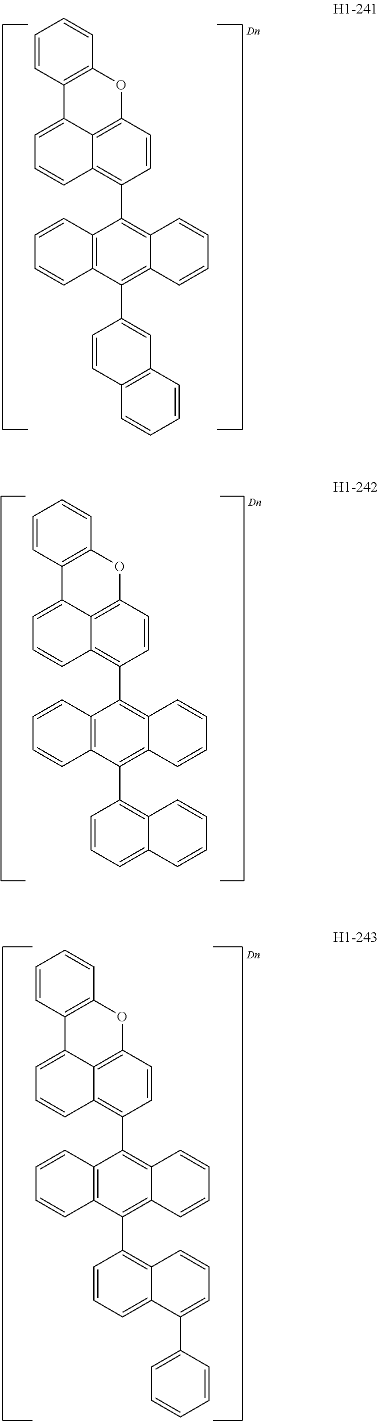 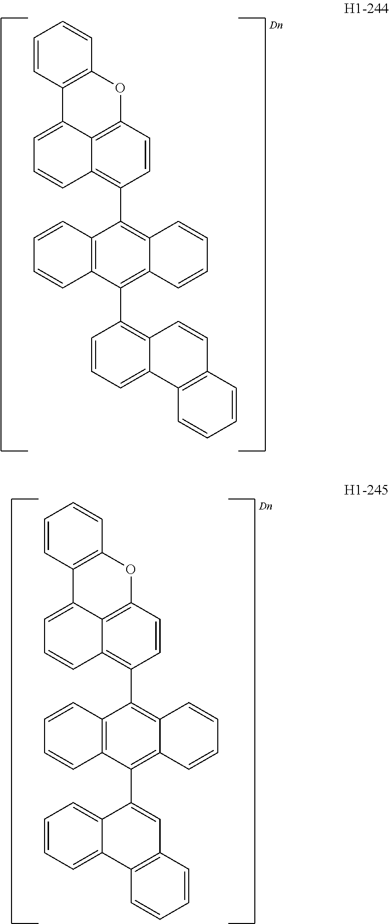 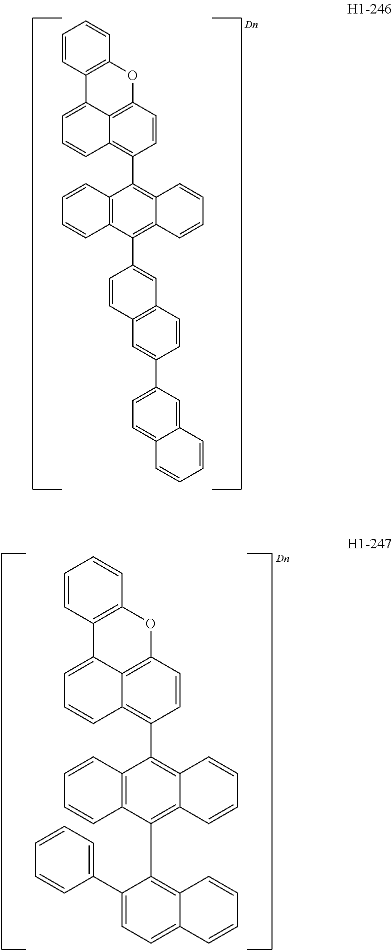 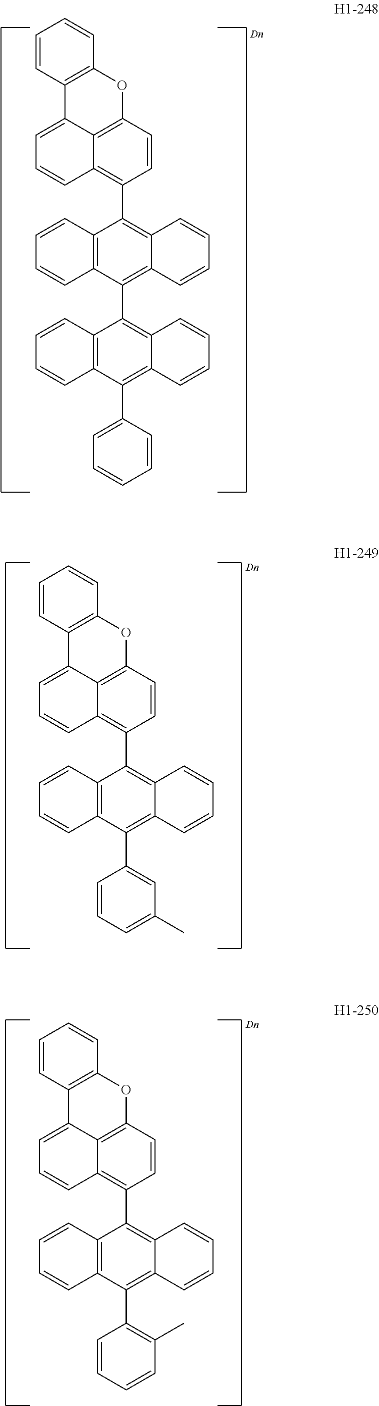 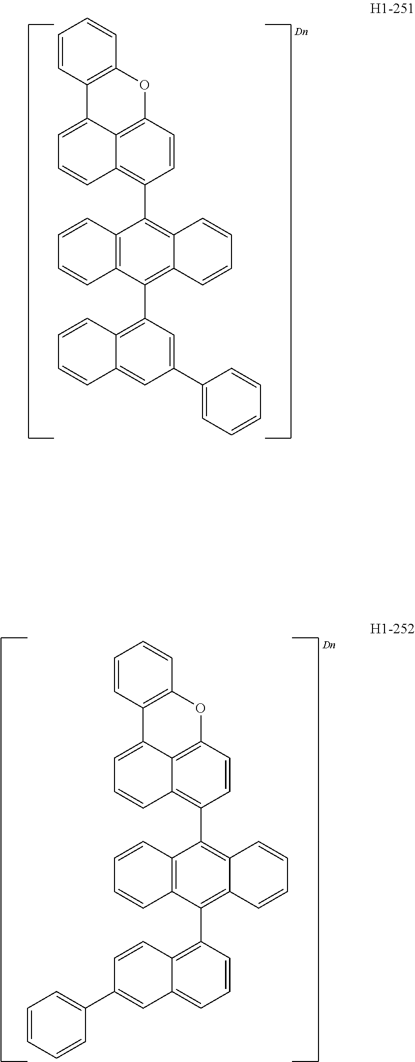 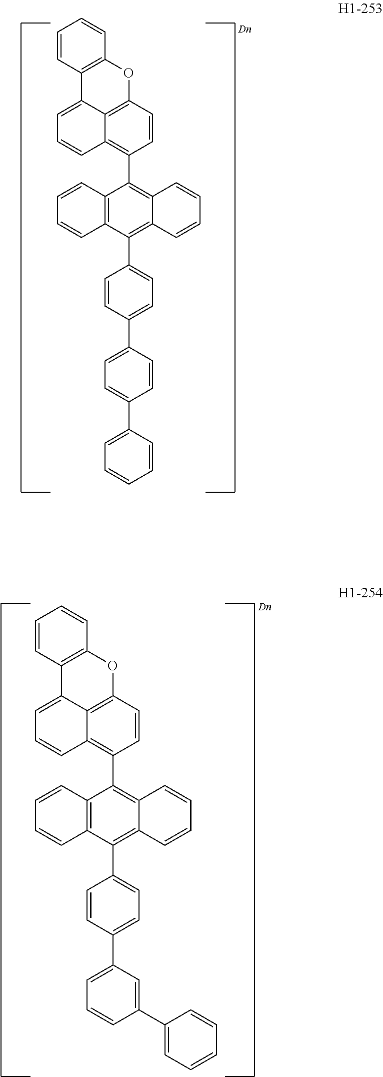 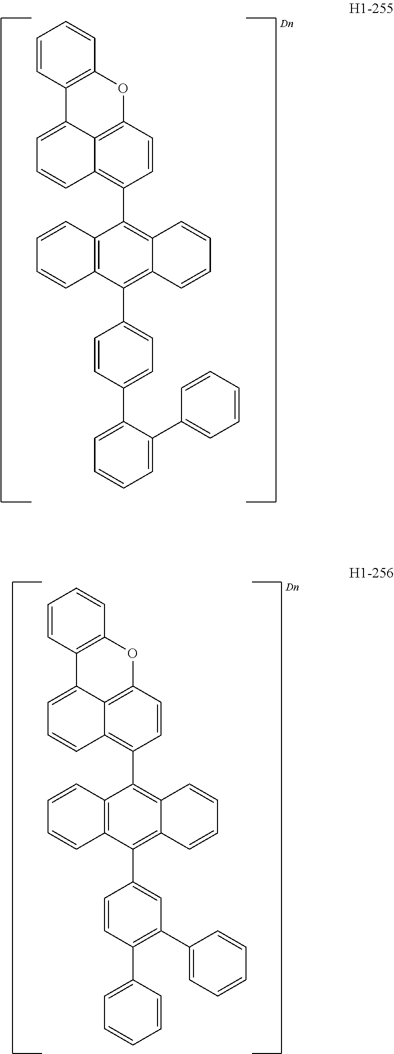 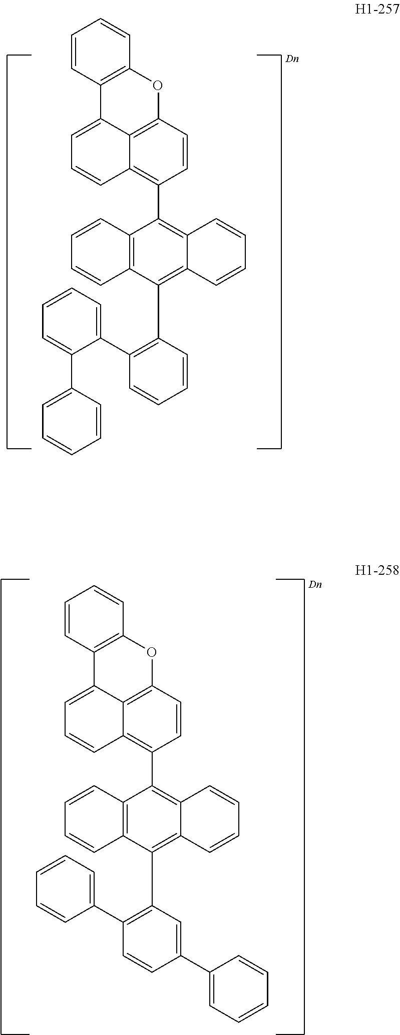 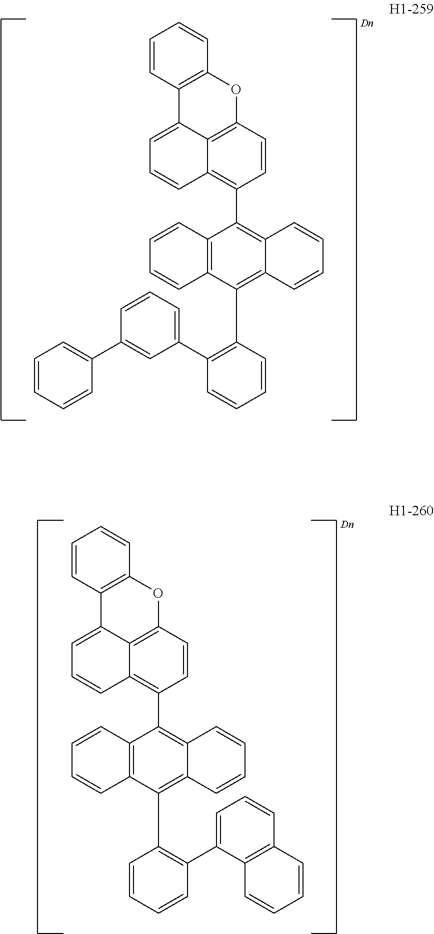 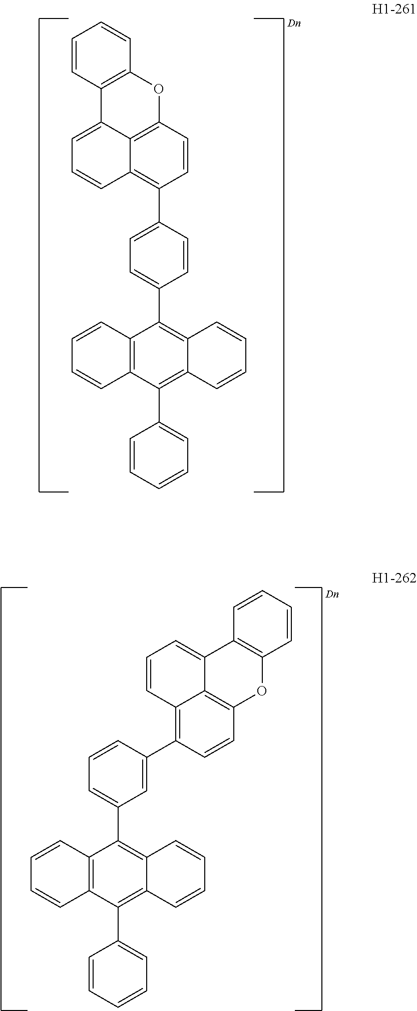 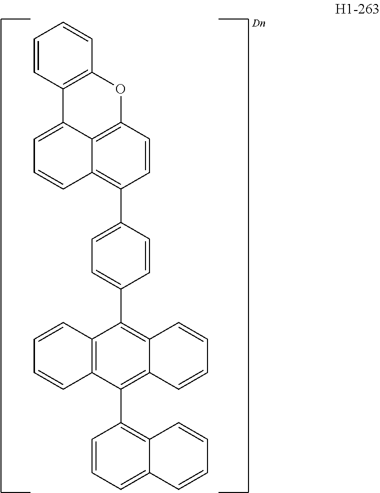 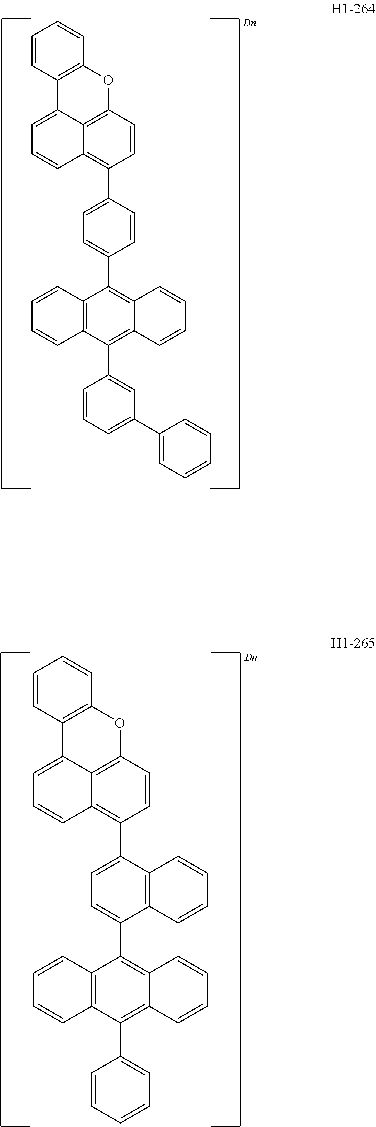 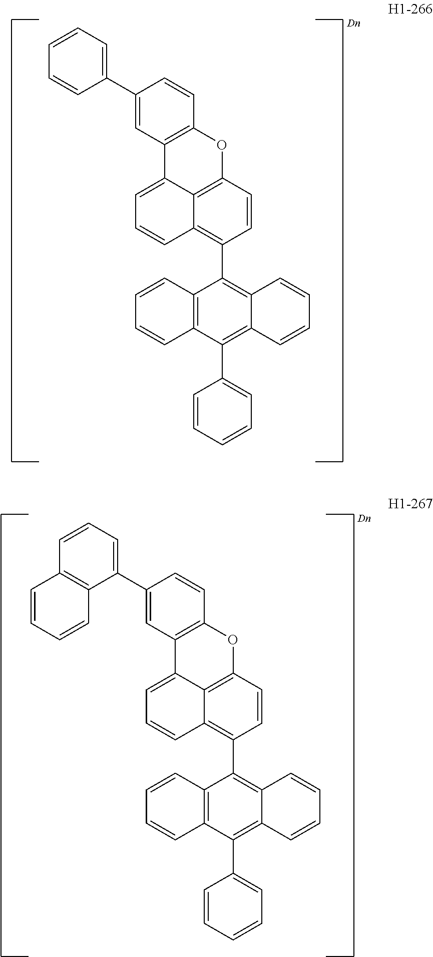 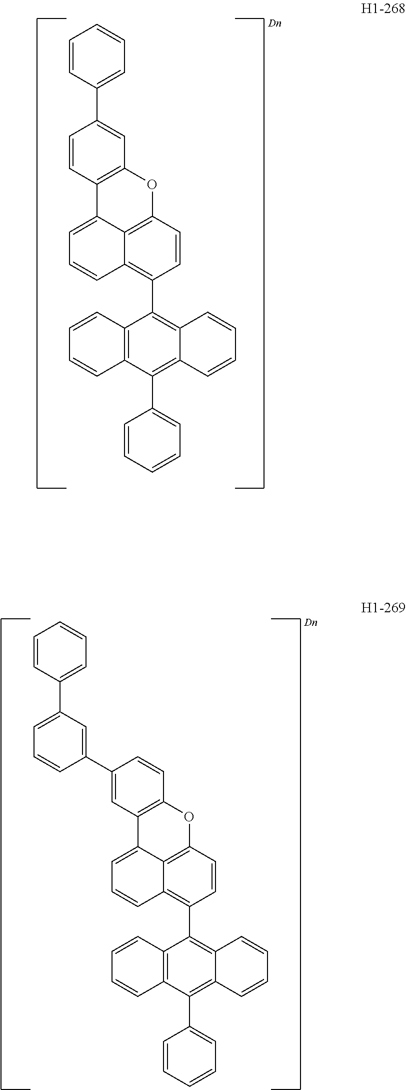 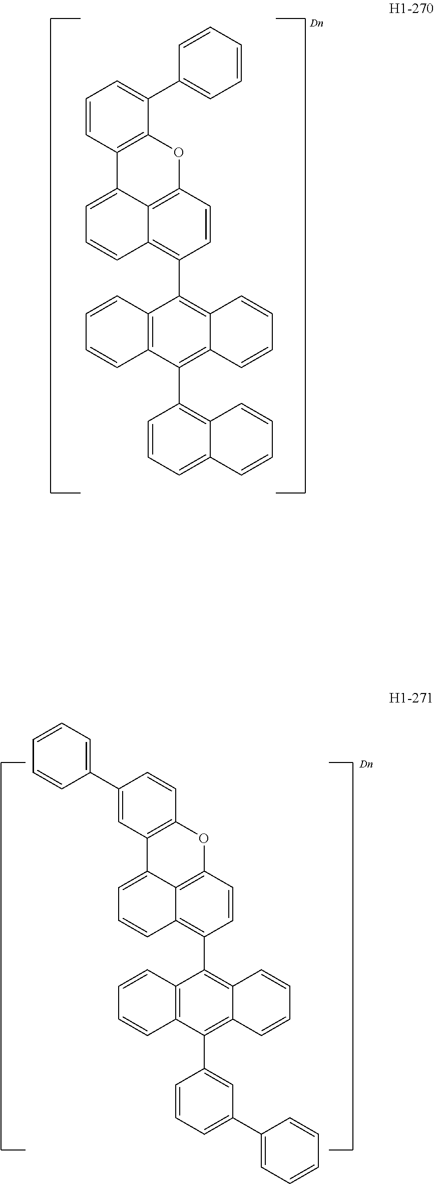 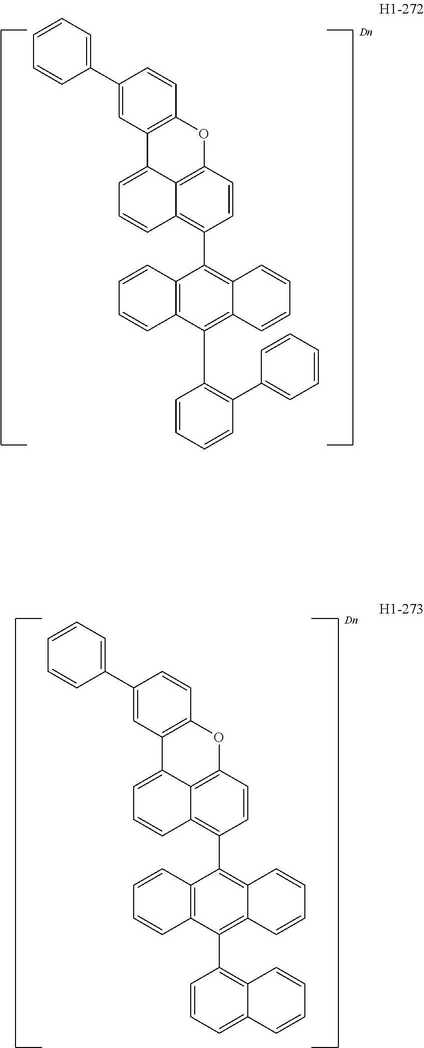 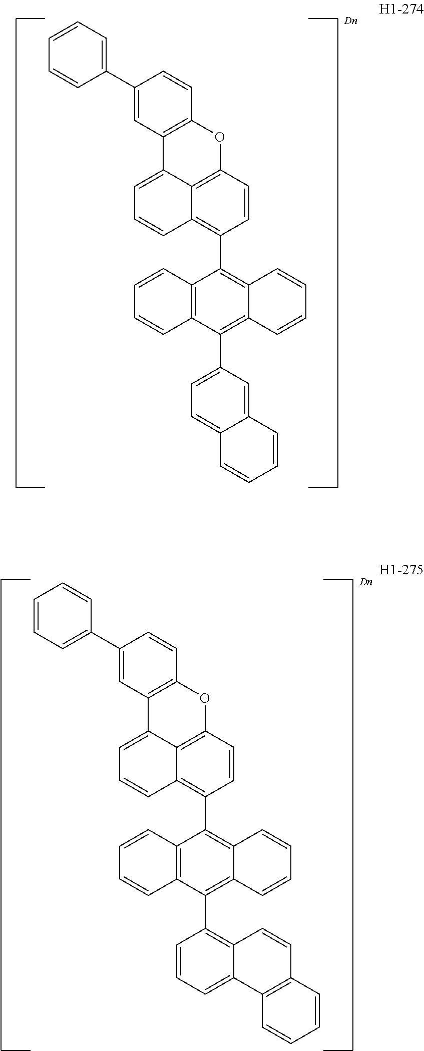 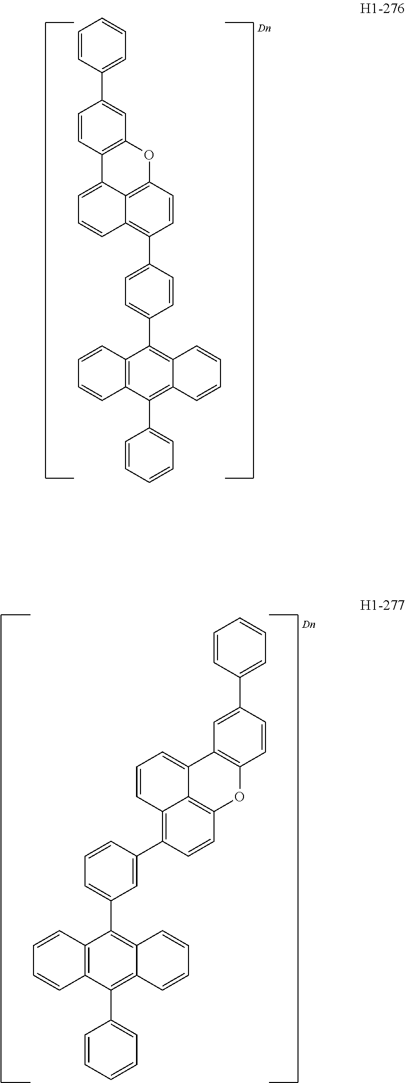 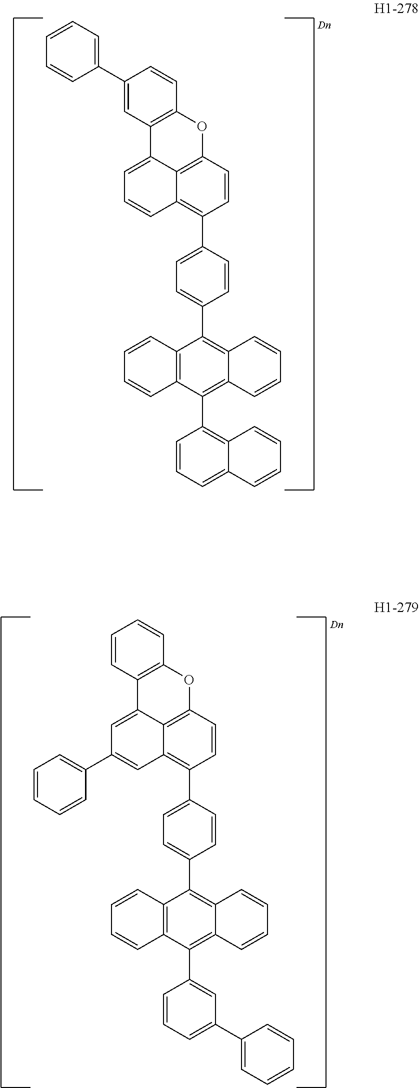 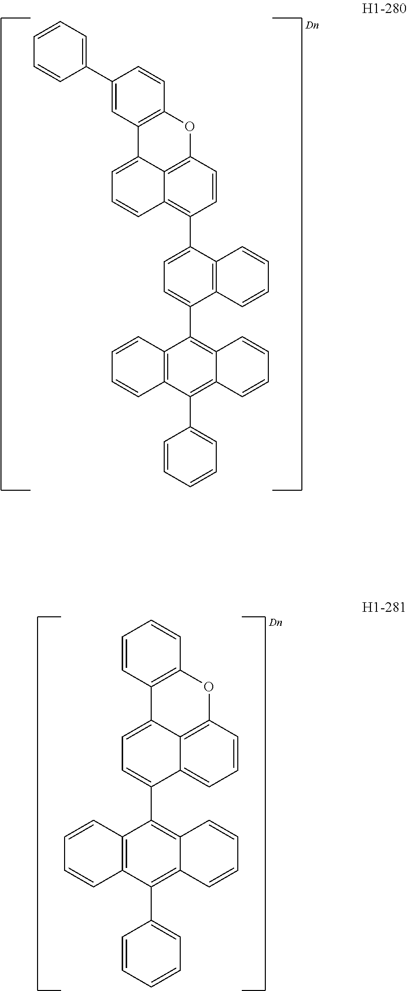 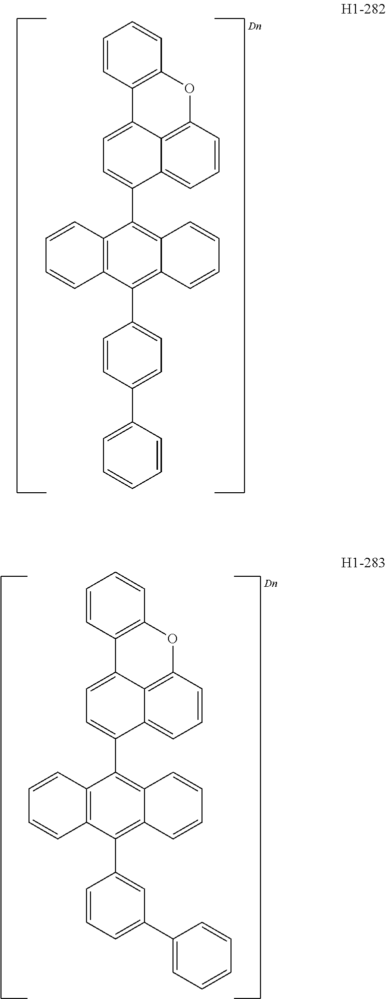 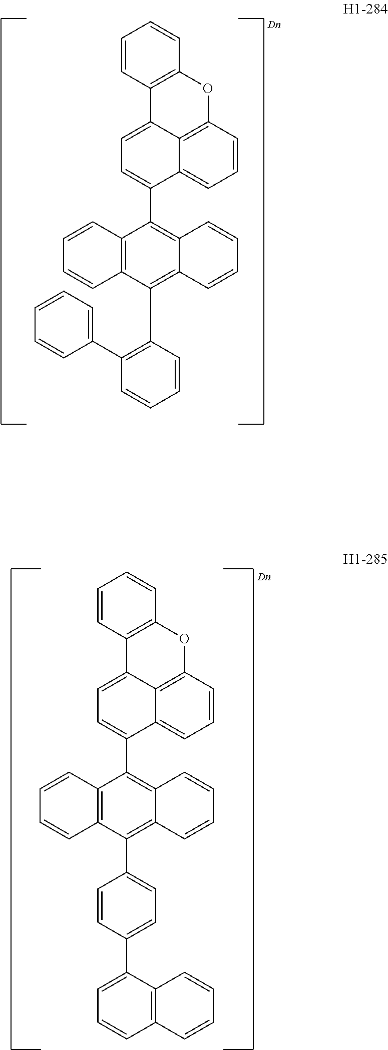 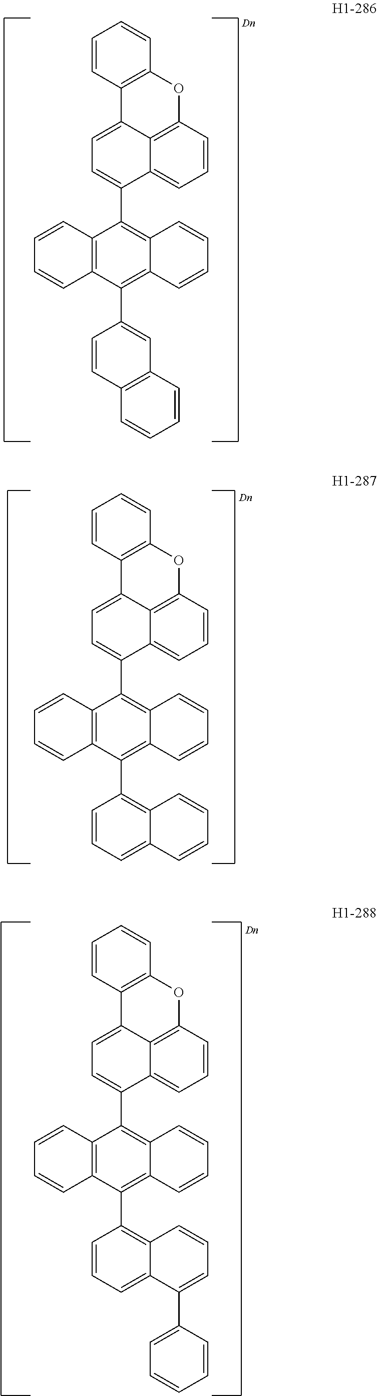 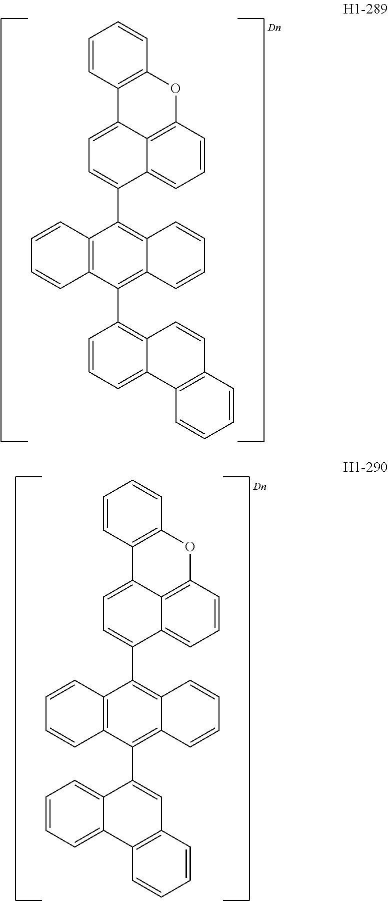 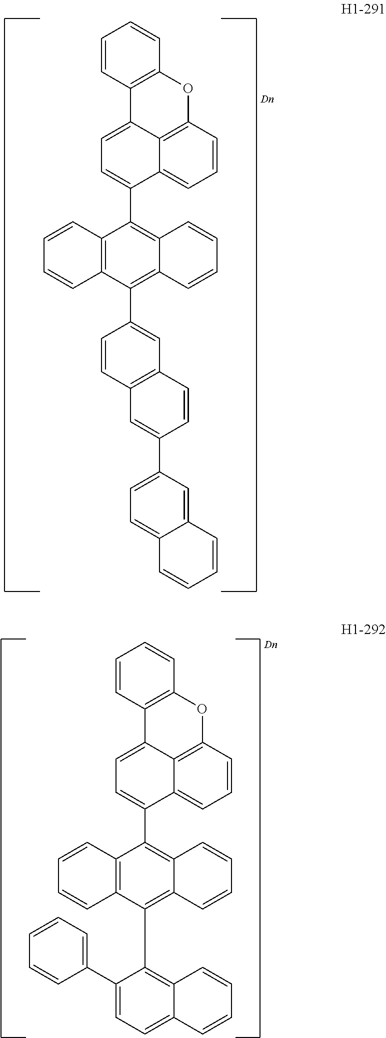 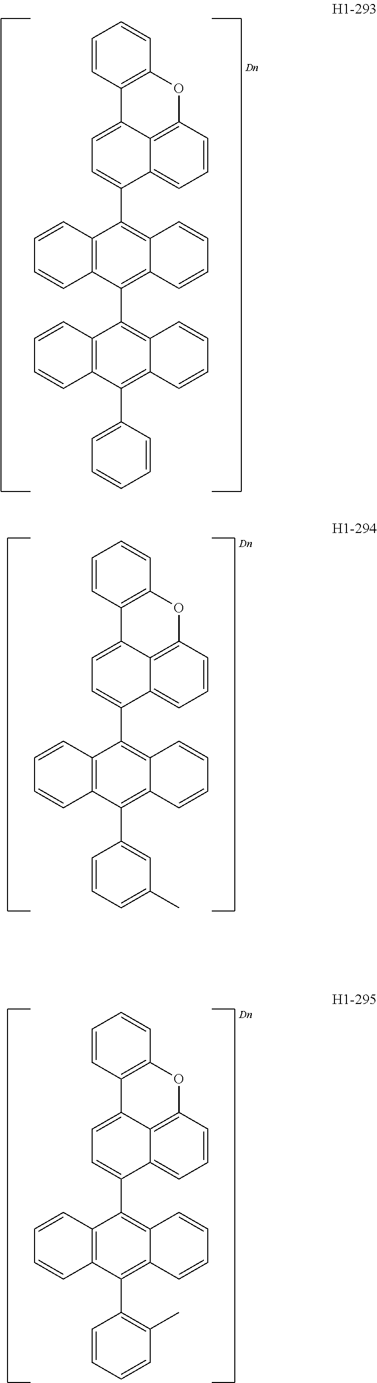 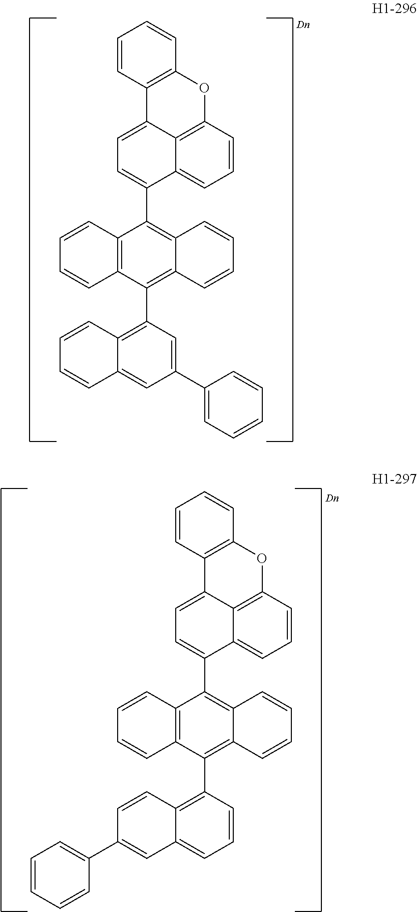 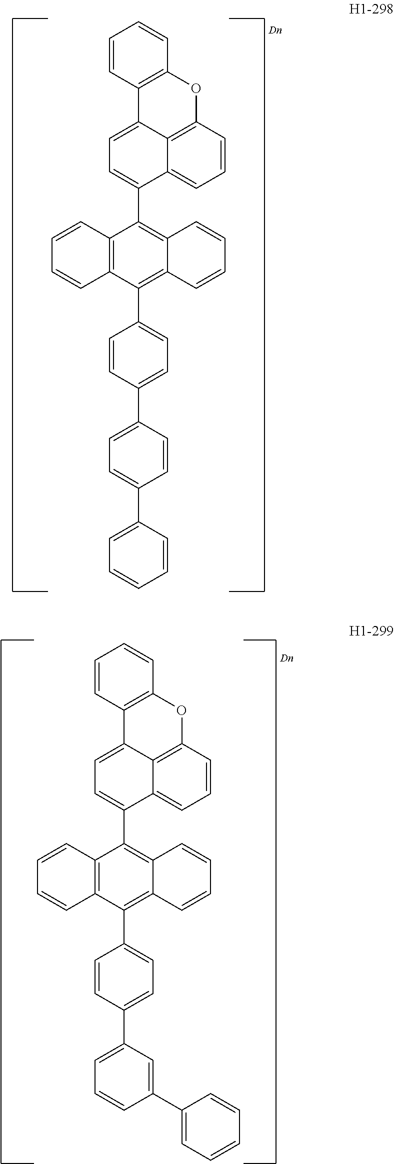 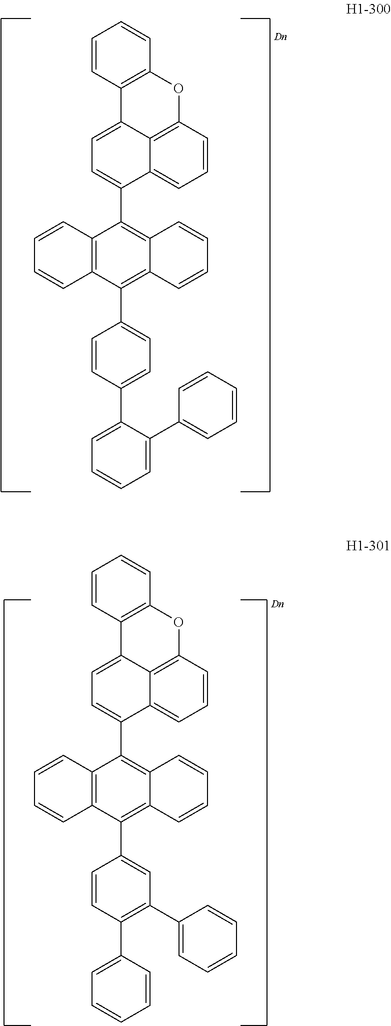 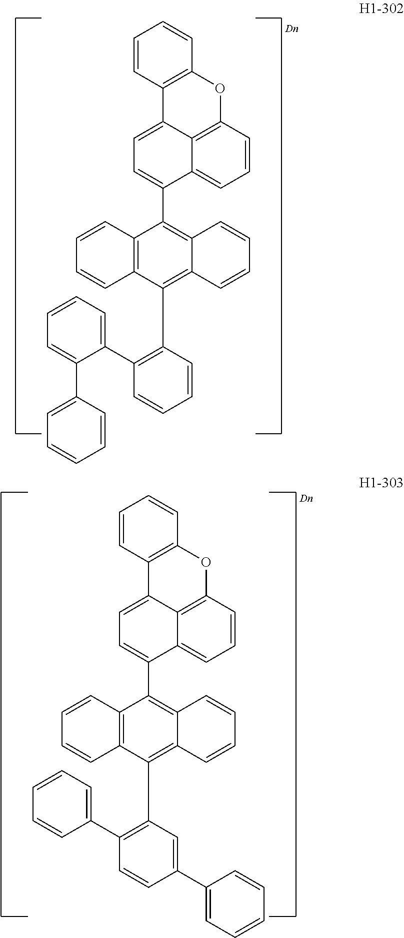 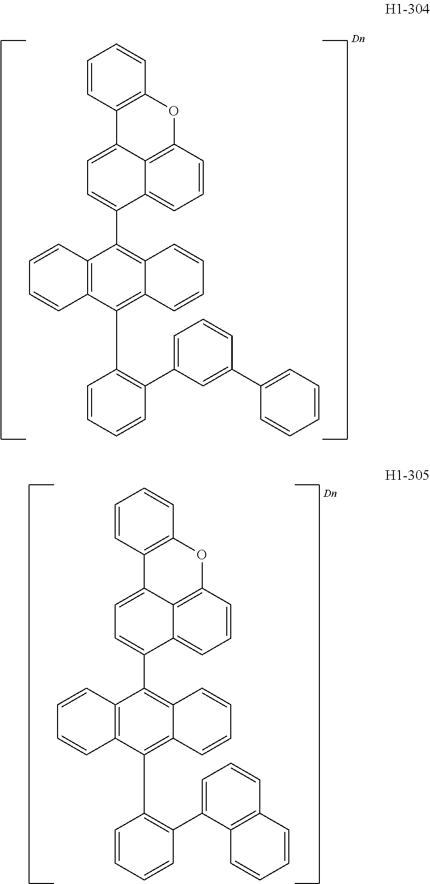 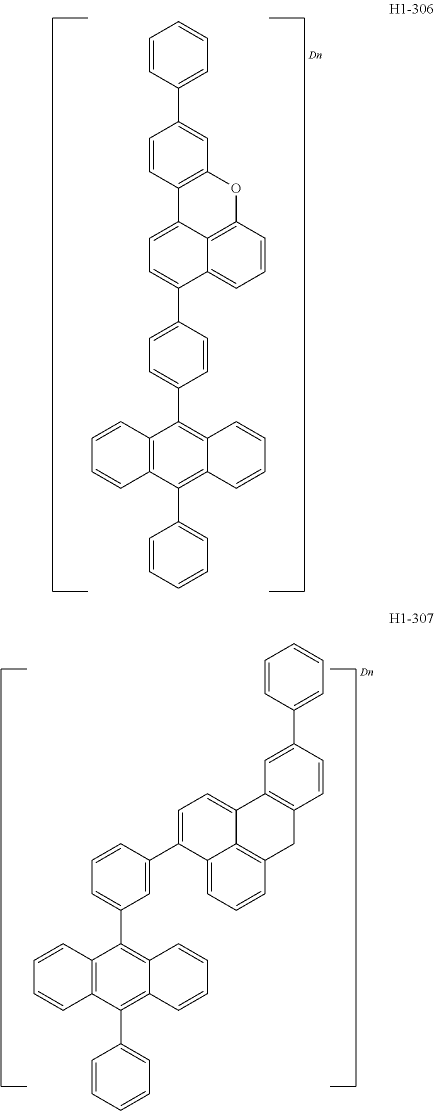 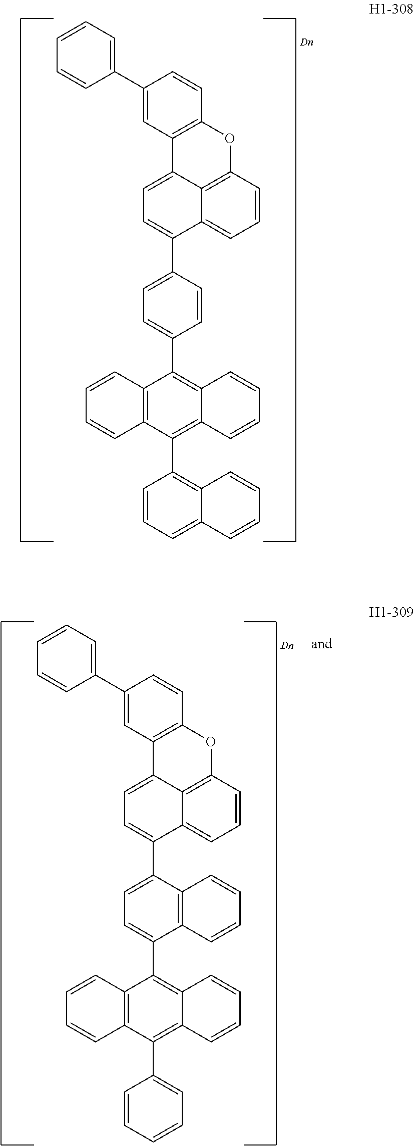 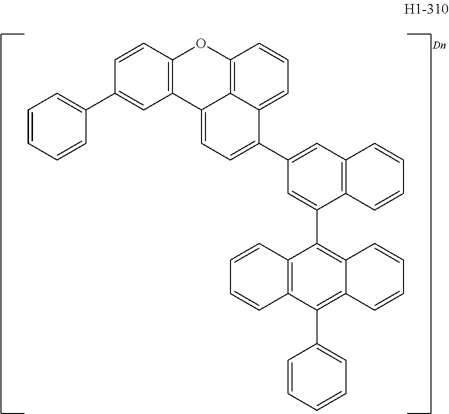
wherein “Dₙ” in the above compounds means that n number of hydrogens is replaced by deuterium, wherein n represents an integer of 1 or more and the upper limit of n is determined by the number of hydrogens that can be substituted in each compound.

8. The plurality of host materials according to claim 1, wherein the second host compound represented by Formula 2 is selected from the following compounds: 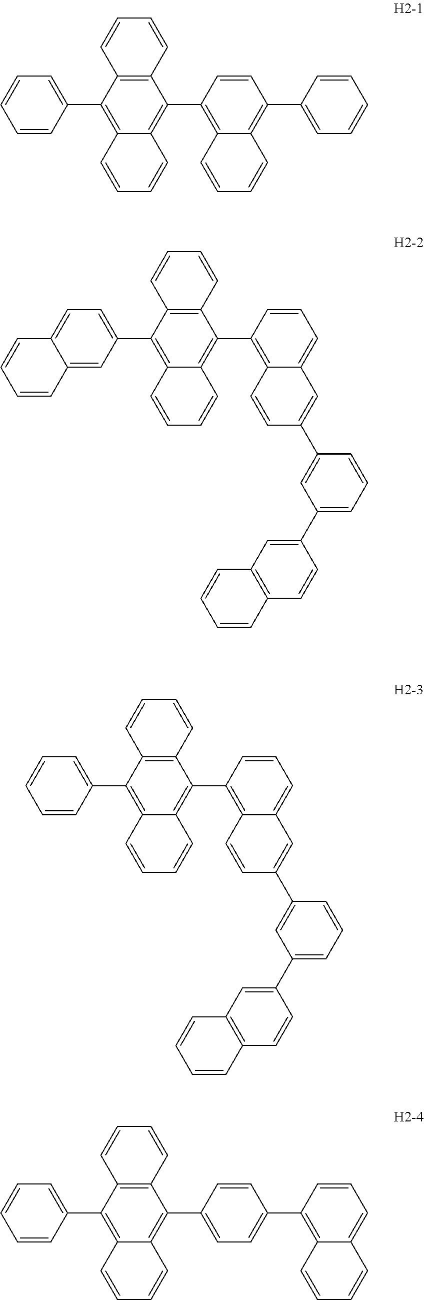 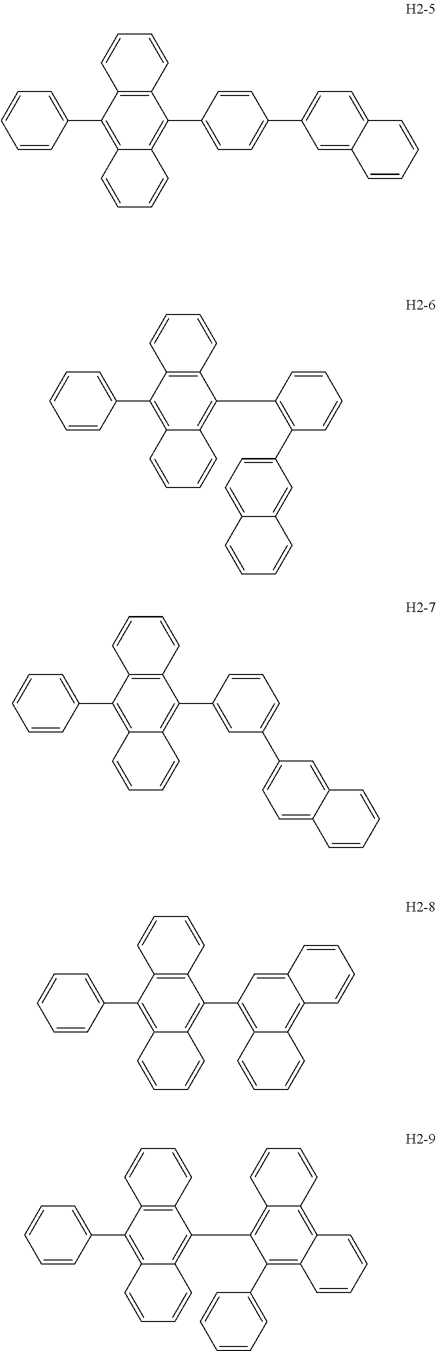 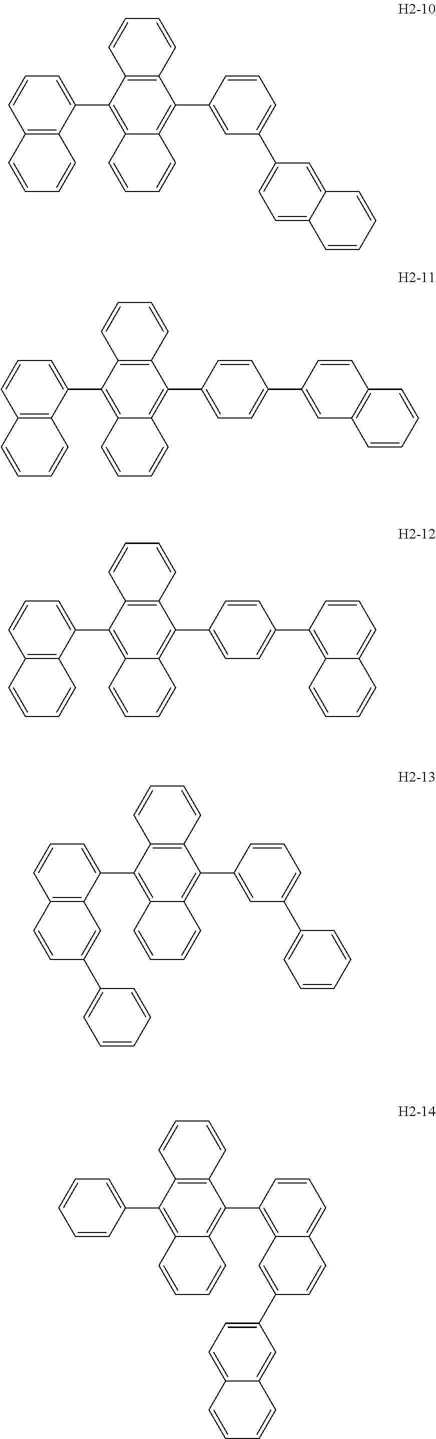 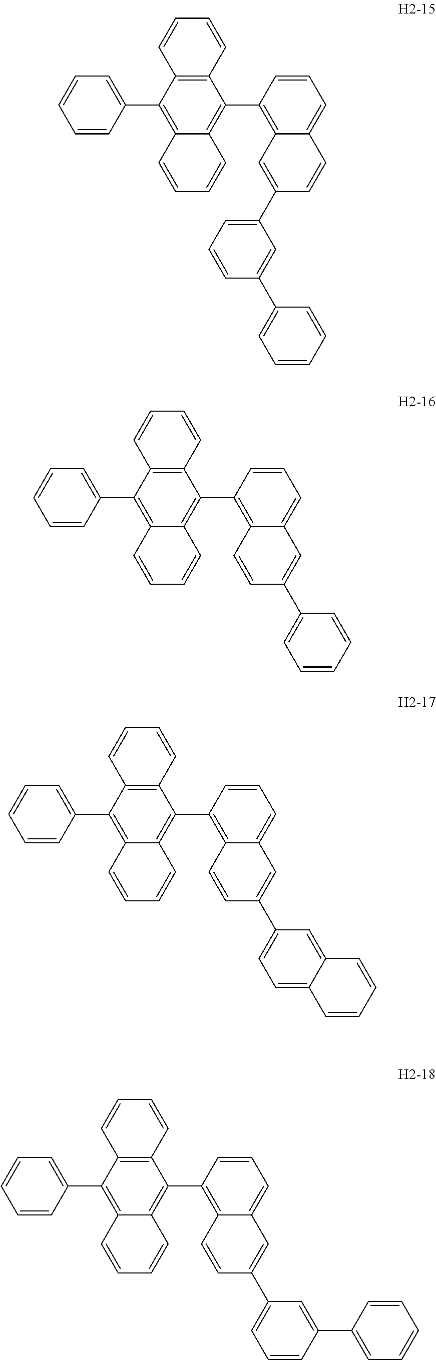 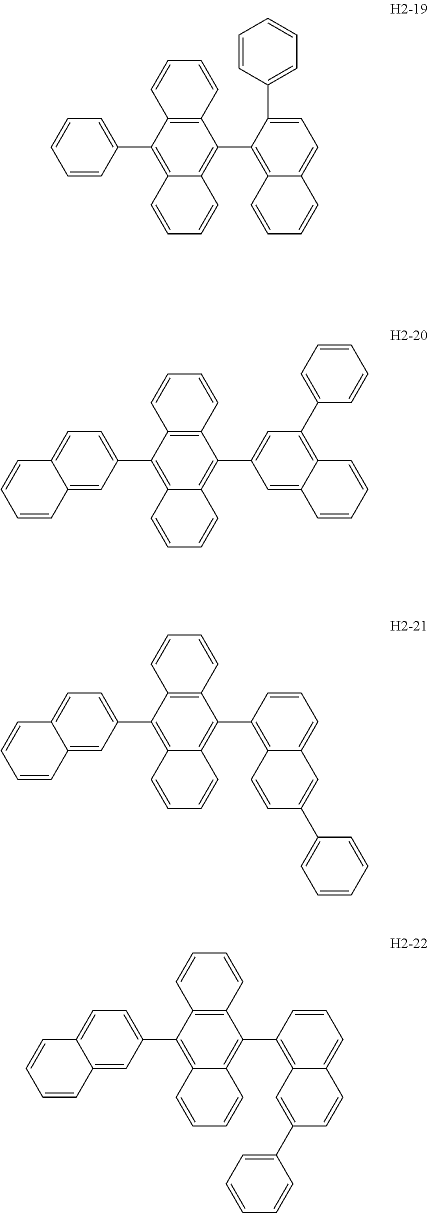 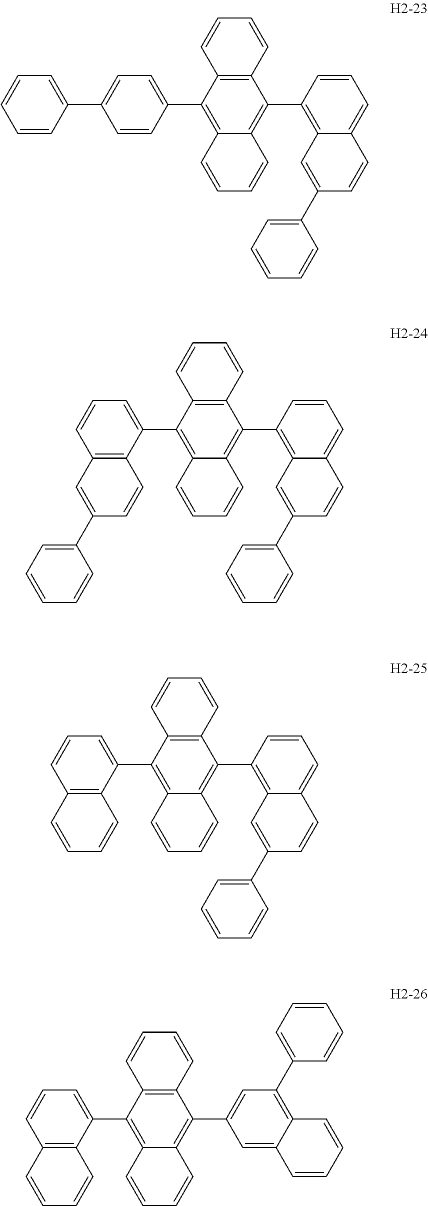 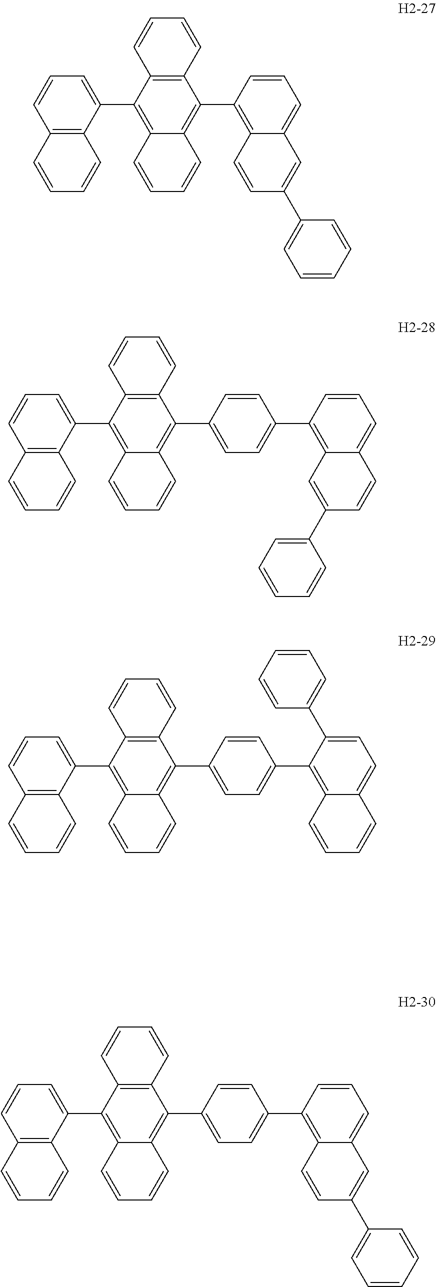 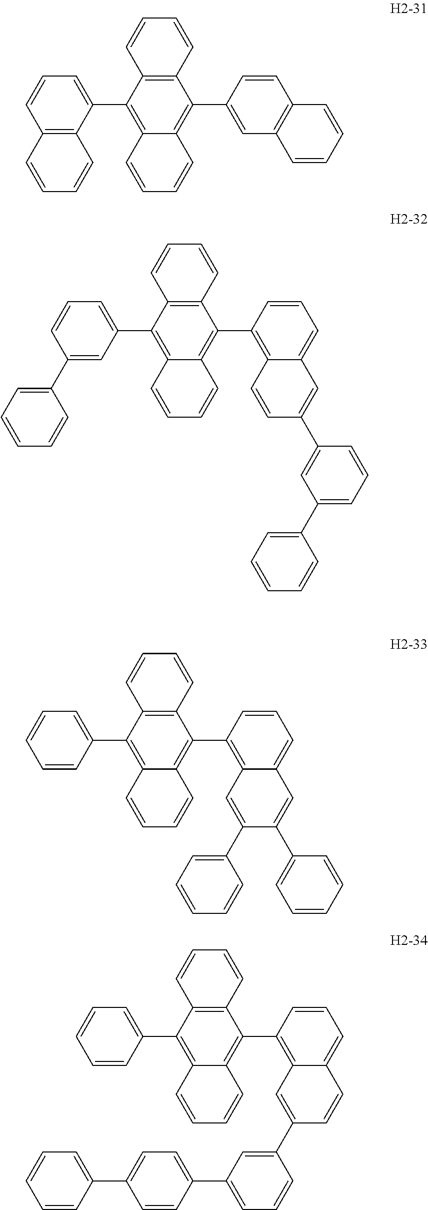 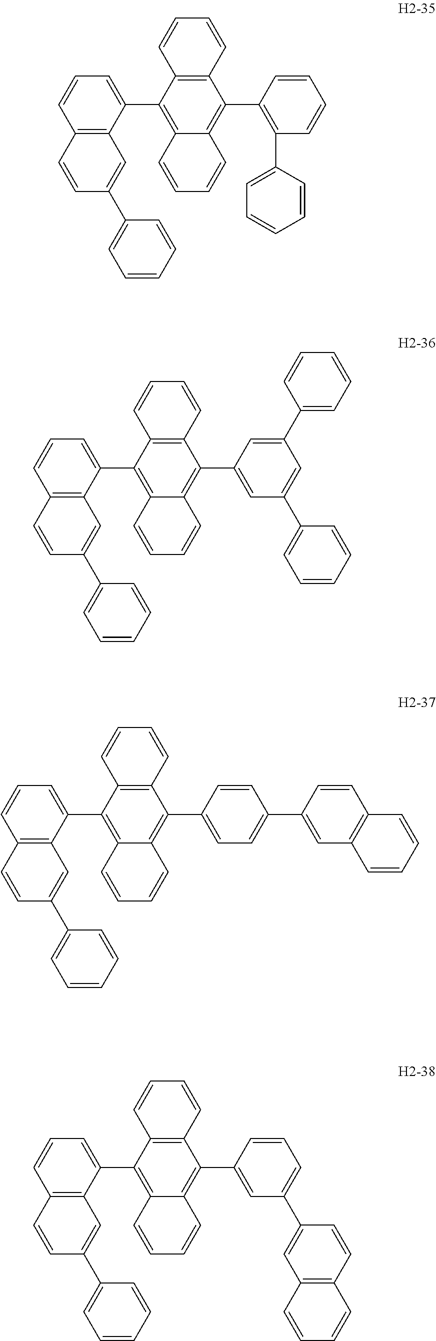 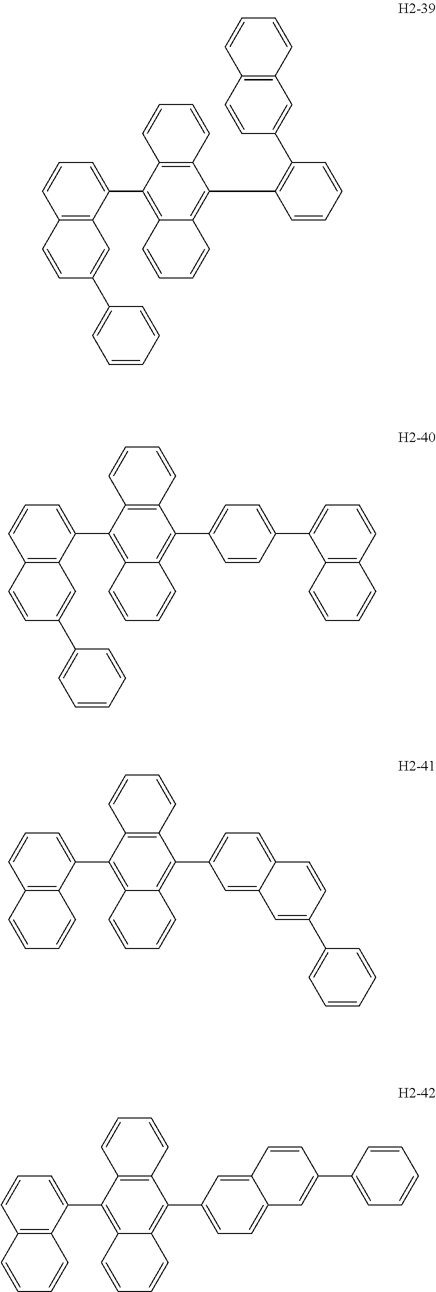 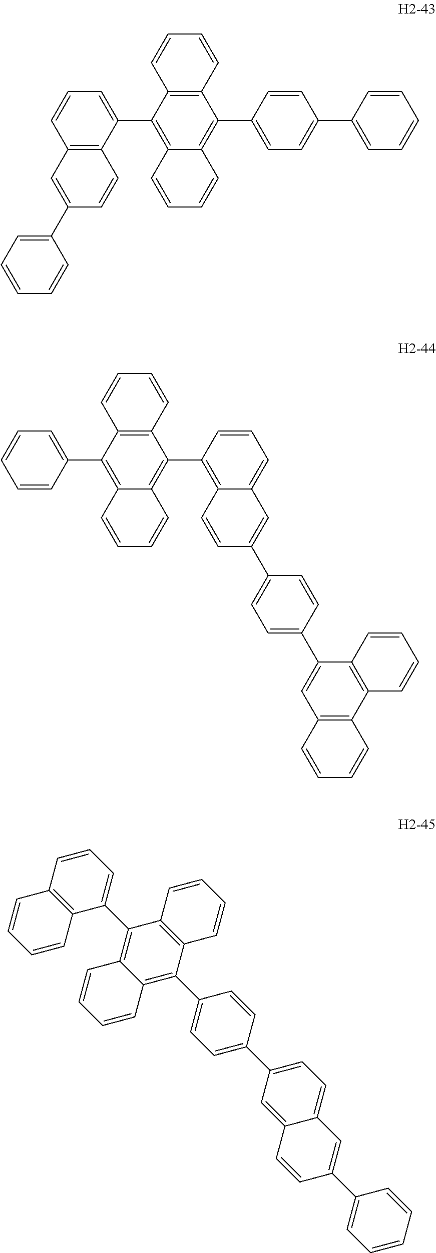 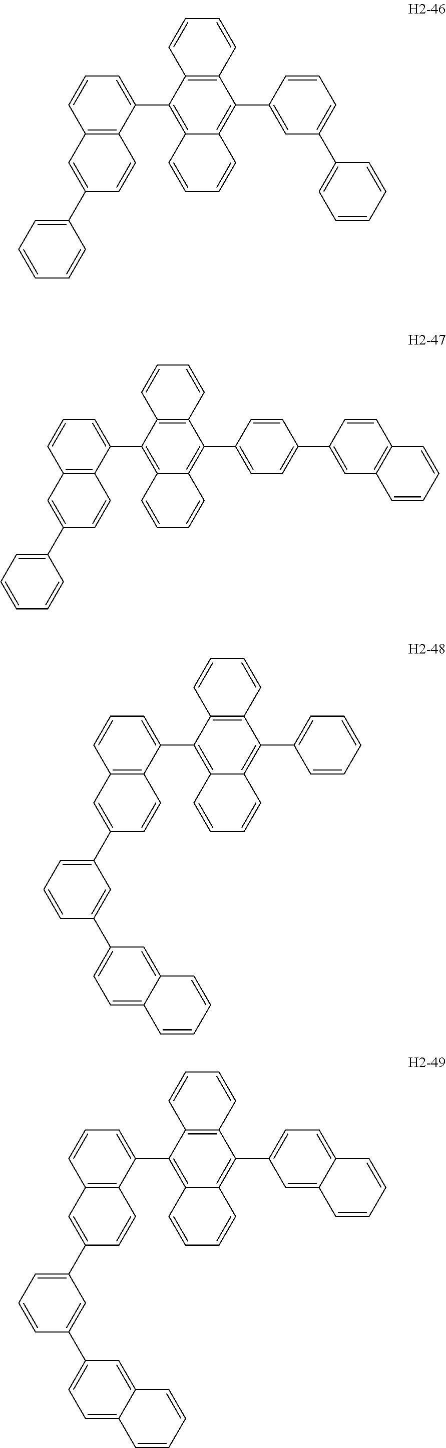 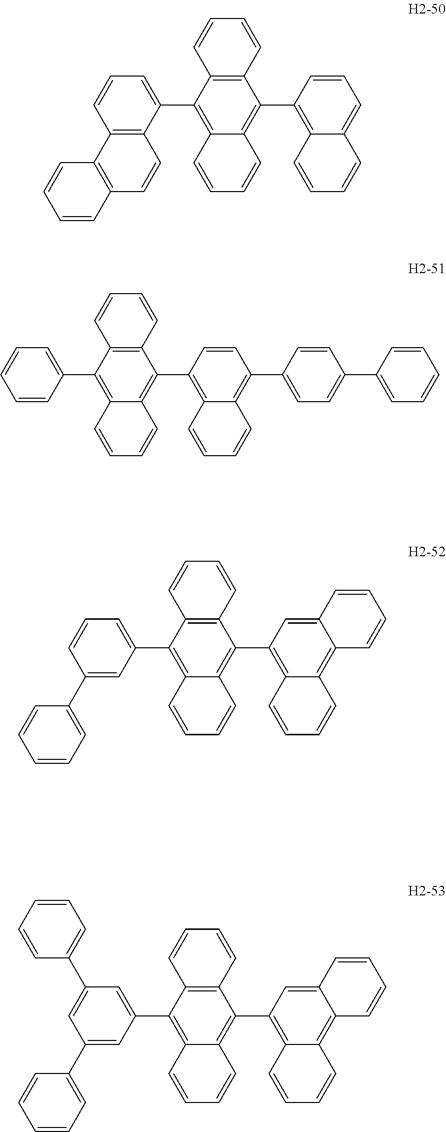 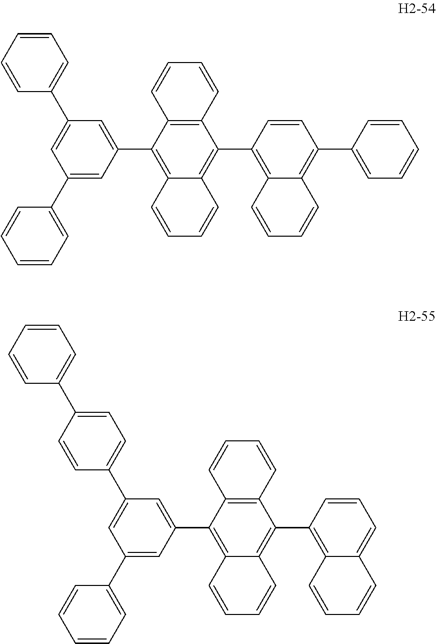 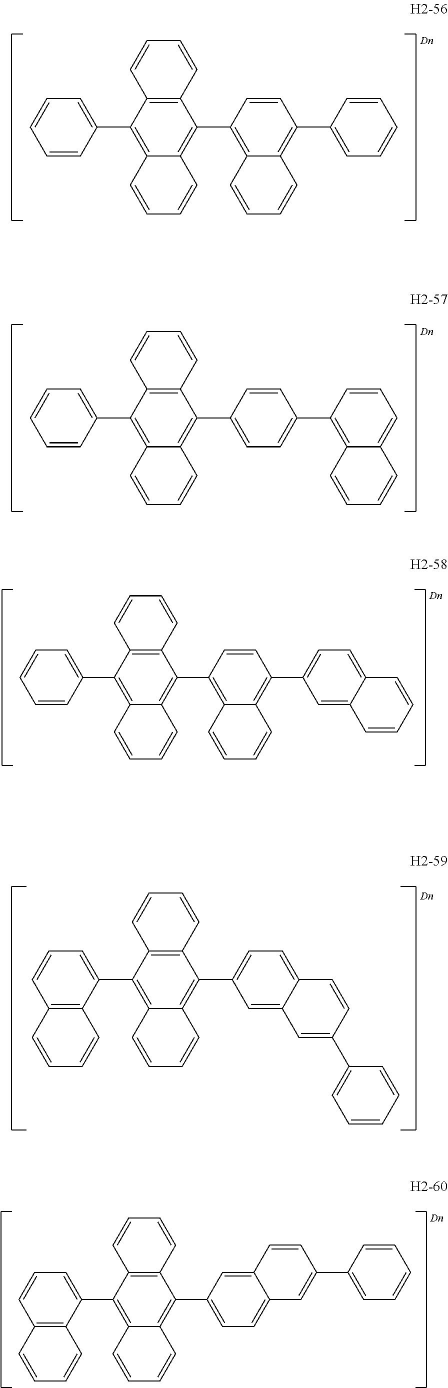 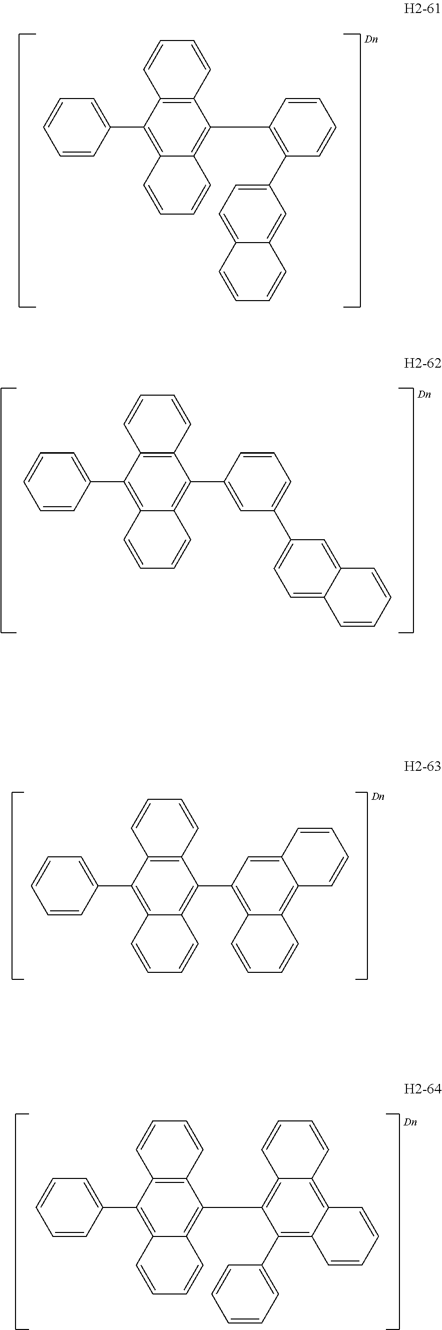 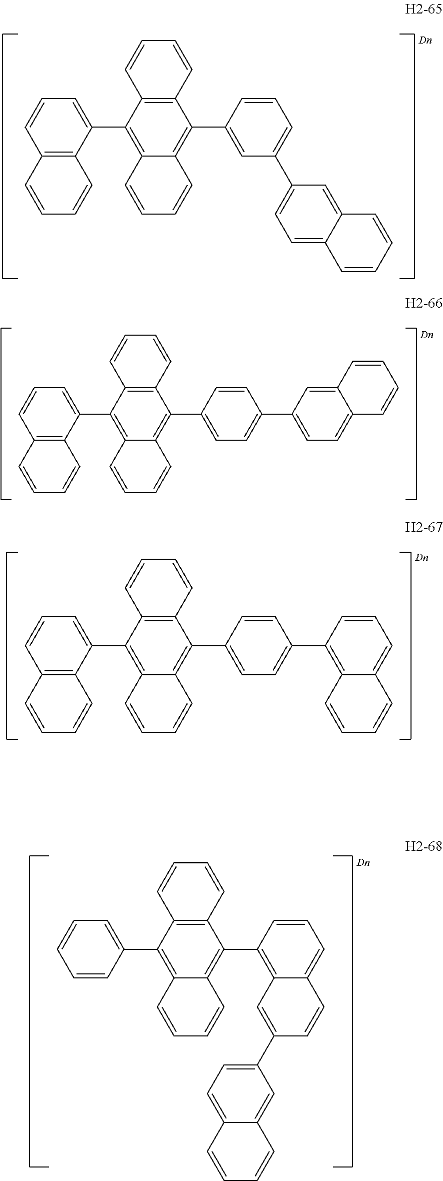 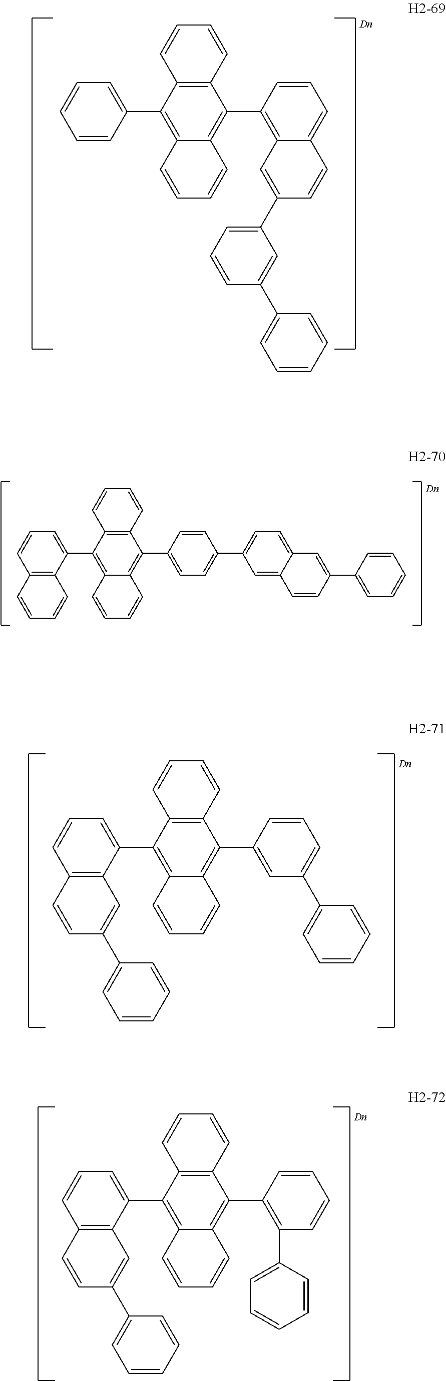 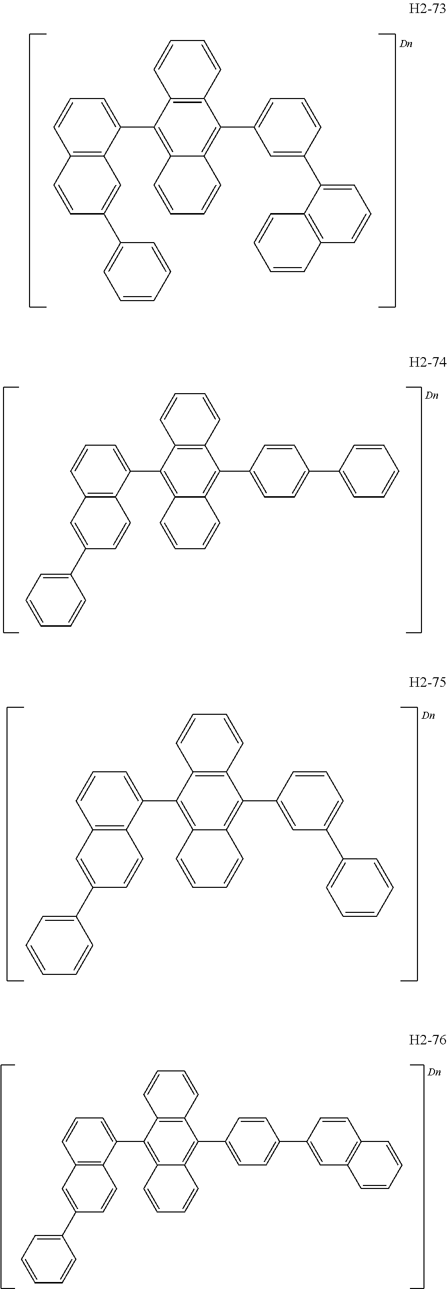 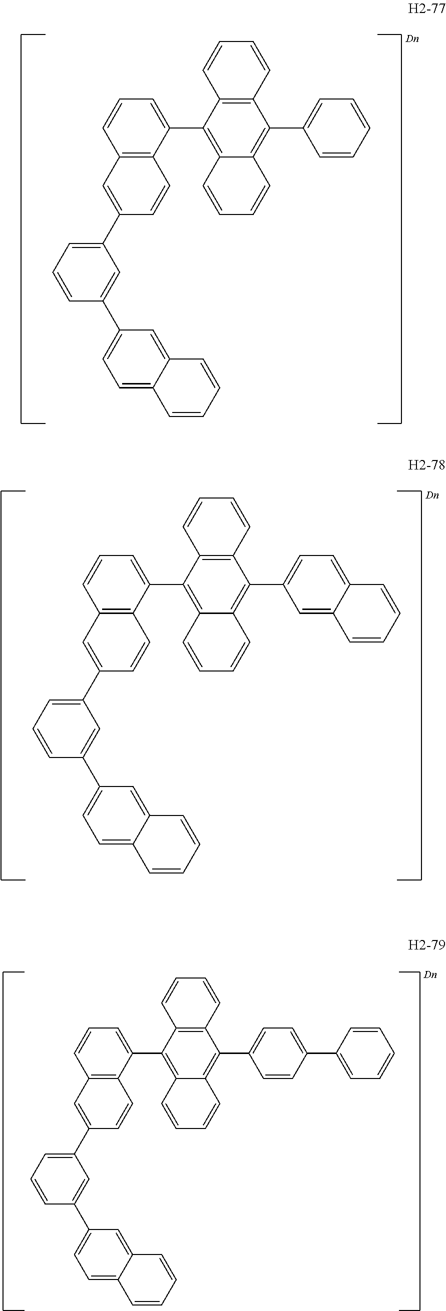 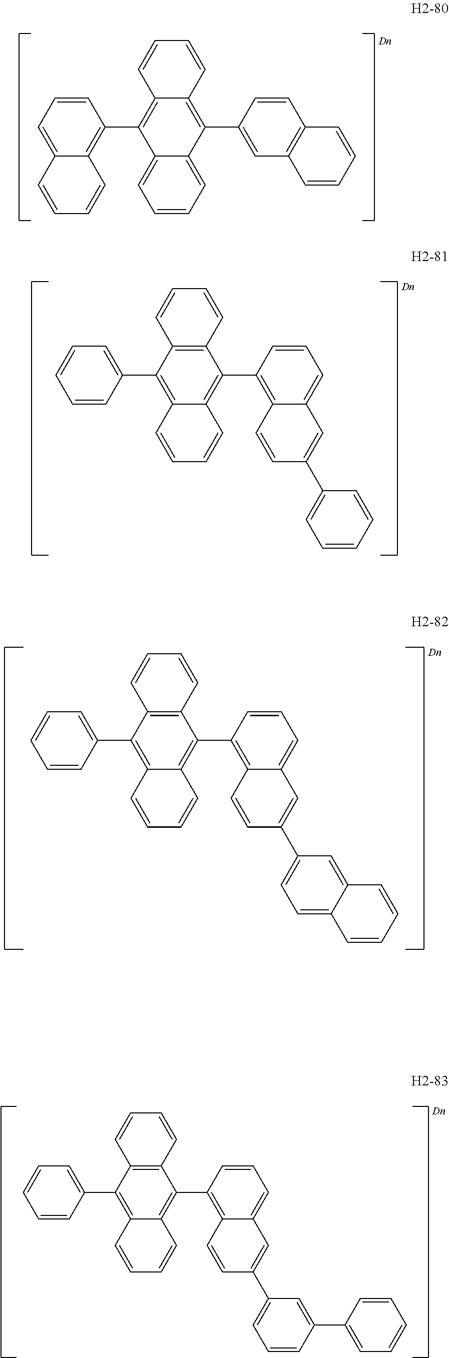 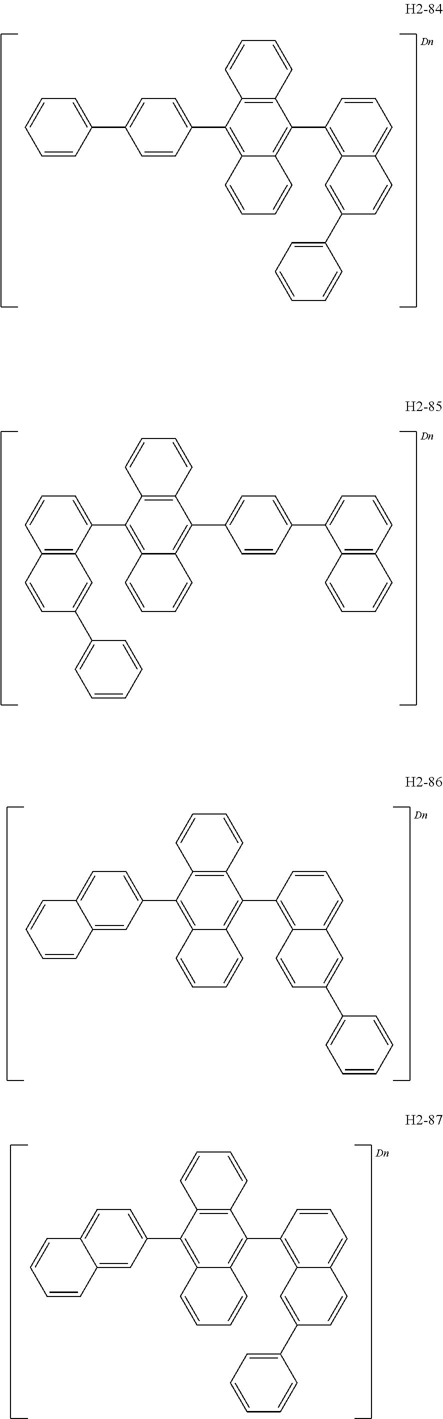 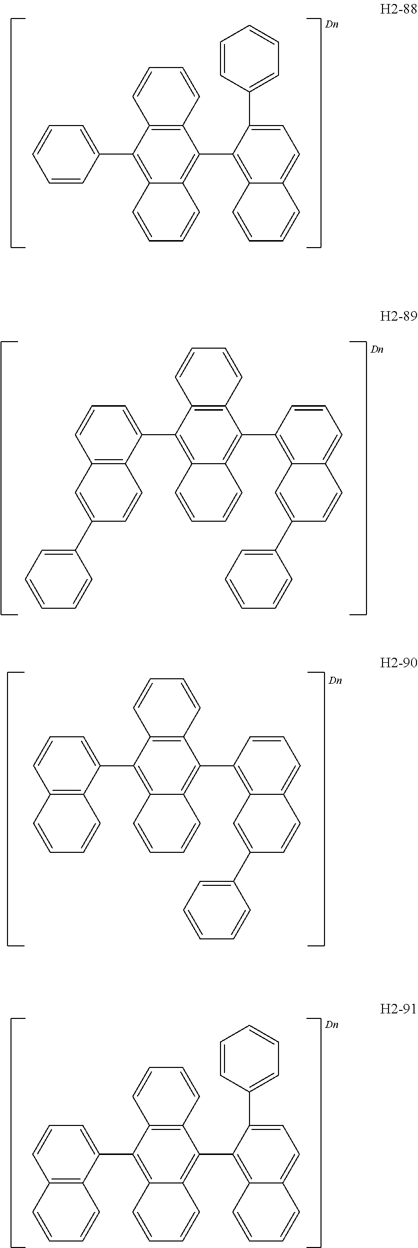 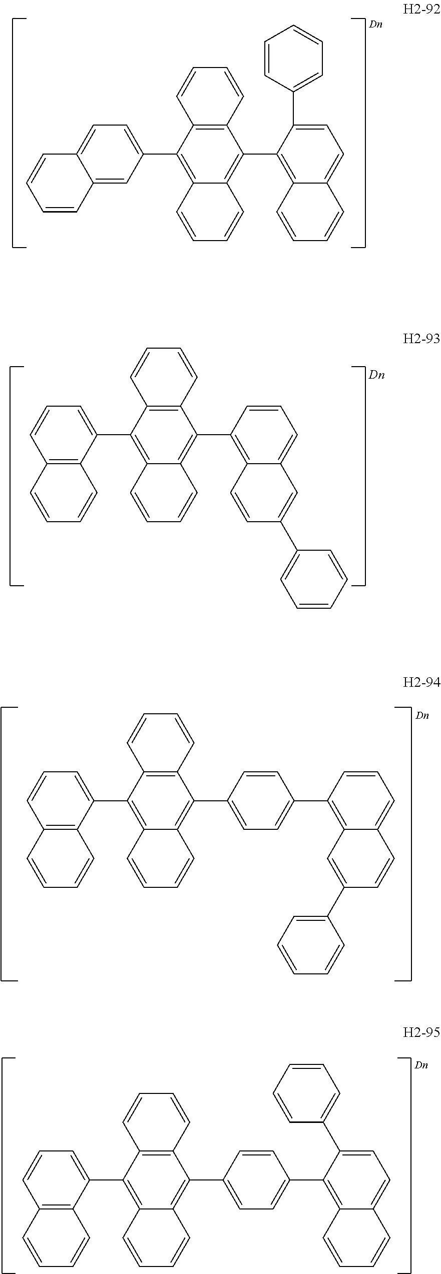 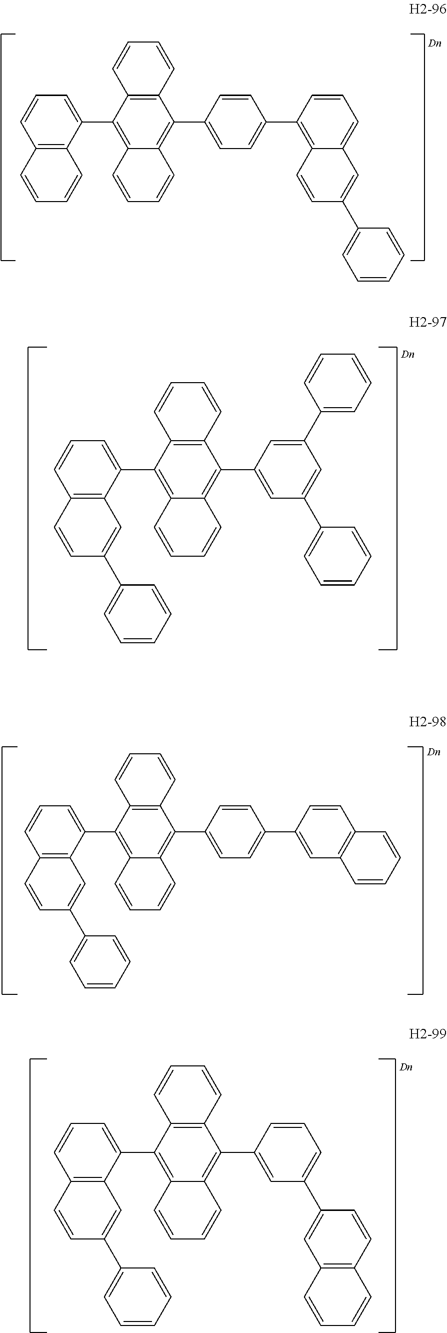 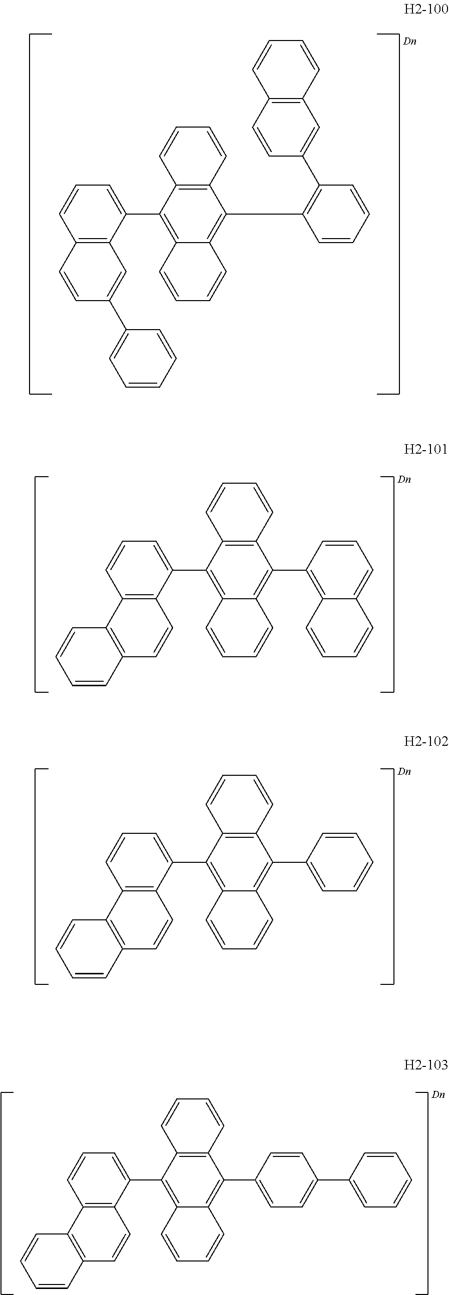 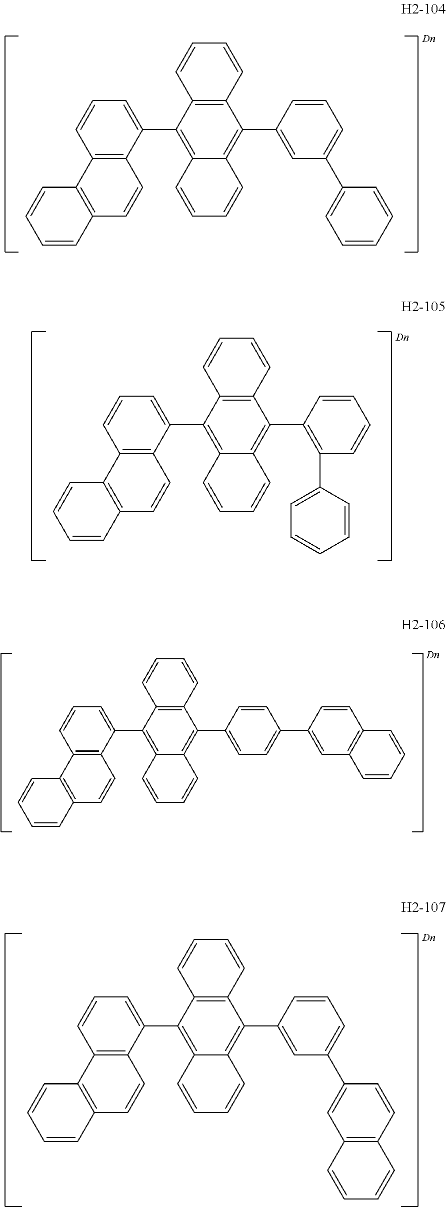 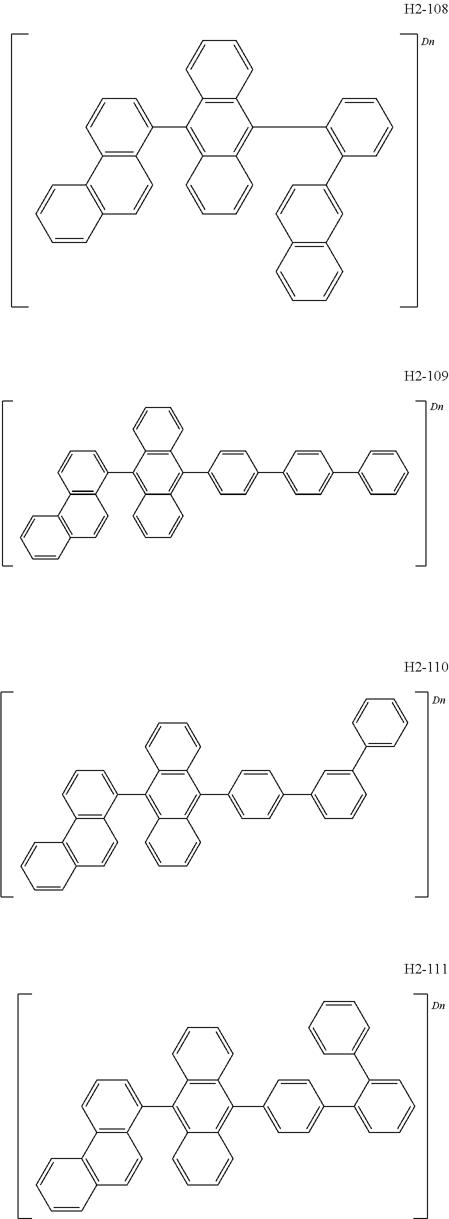 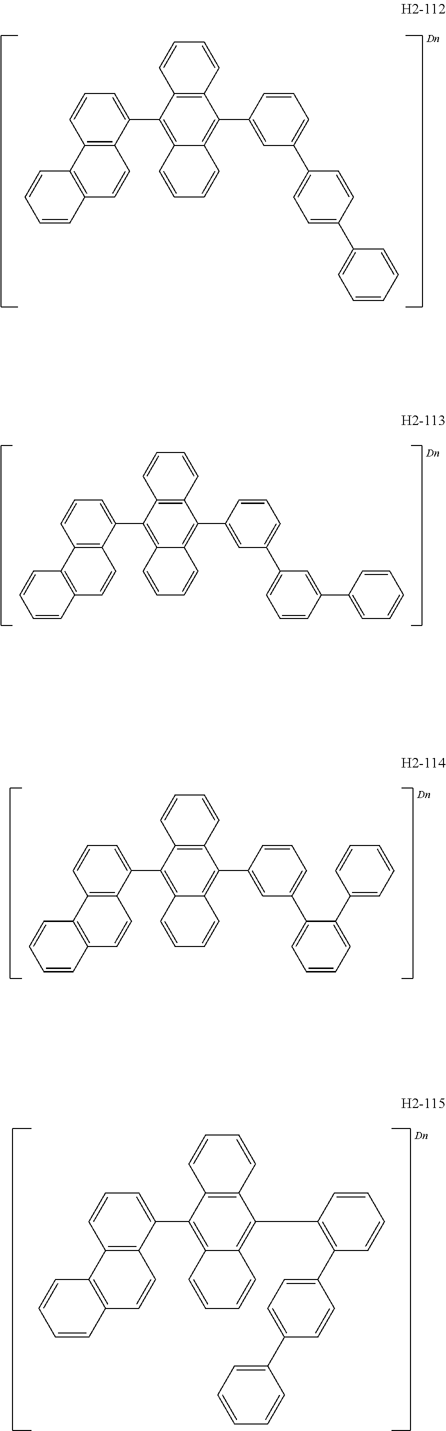 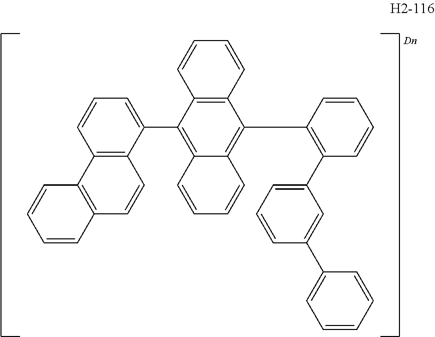 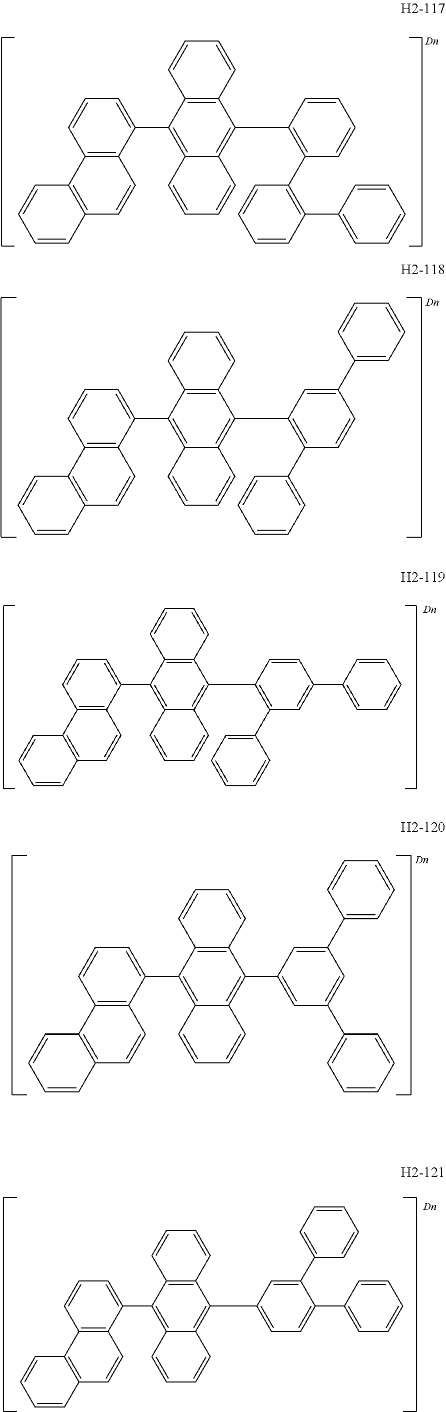 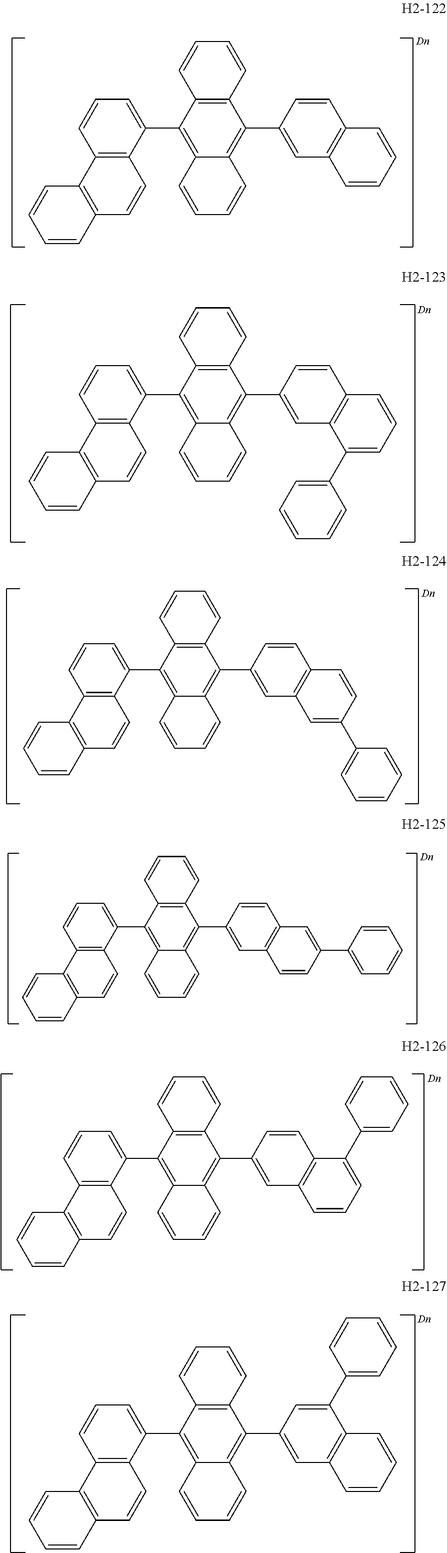 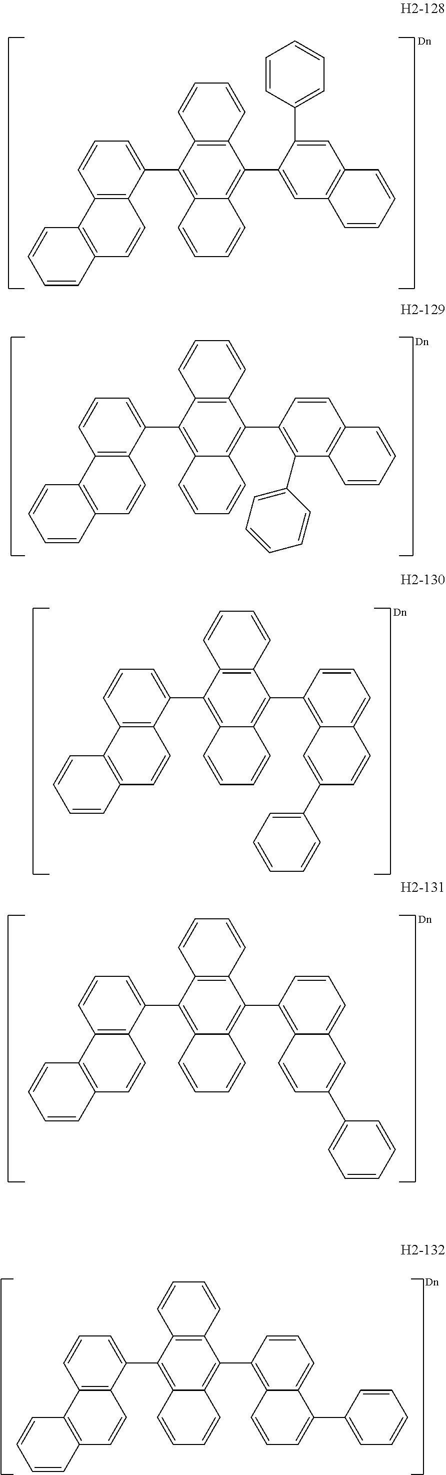 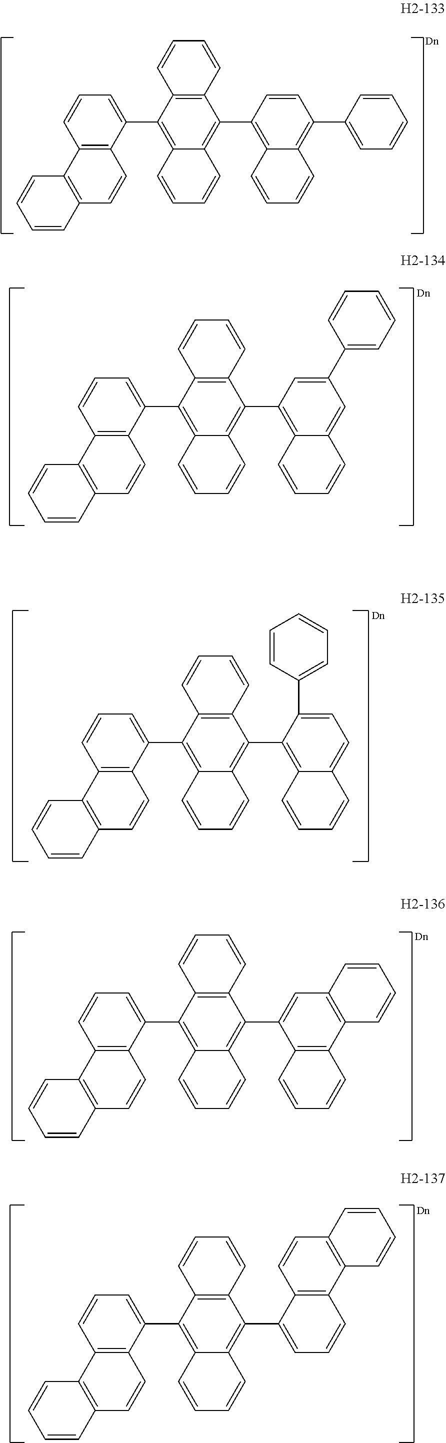 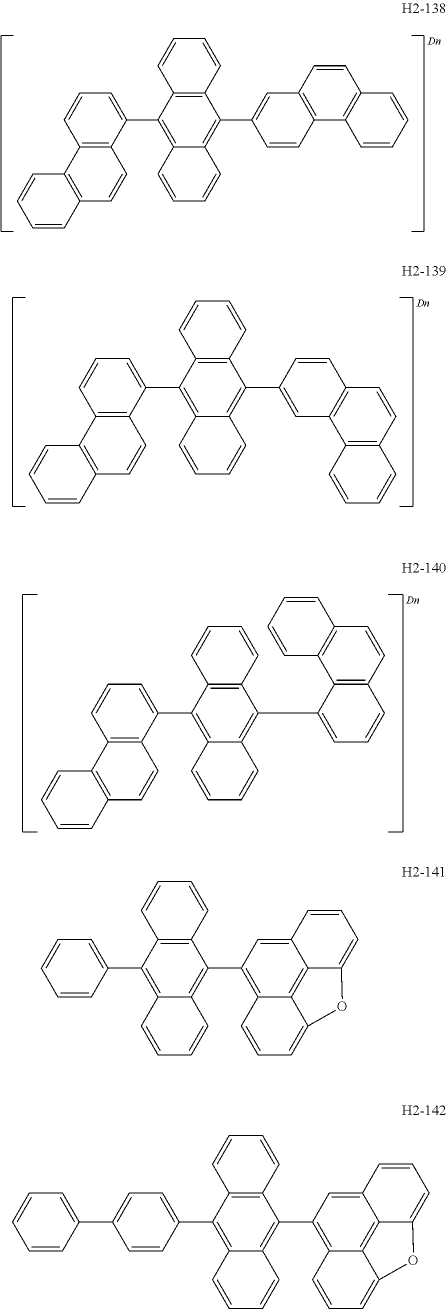 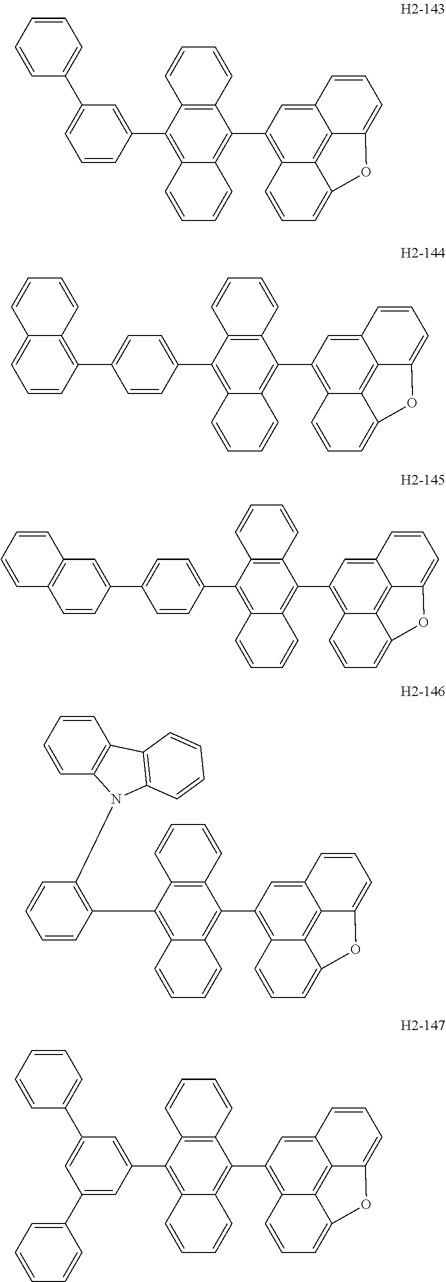 [SMILES: c1ccc(-c2cccc(-c3c4ccccc4c(-c4cc5cccc6oc7cccc4c7c56)c4ccccc34)c2)cc1 ; c1ccc2c(-c3ccc(-c4c5ccccc5c(-c5cc6cccc7oc8cccc5c8c67)c5ccccc45)cc3)cccc2c1 ; c1ccc2cc(-c3ccc(-c4c5ccccc5c(-c5cc6cccc7oc8cccc5c8c67)c5ccccc45)cc3)ccc2c1 ; c1ccc(-n2c3ccccc3c3ccccc32)c(-c2c3ccccc3c(-c3cc4cccc5oc6cccc3c6c45)c3ccccc23)c1 ; c1ccc(-c2cc(-c3ccccc3)cc(-c3c4ccccc4c(-c4cc5cccc6oc7cccc4c7c56)c4ccccc34)c2)cc1] 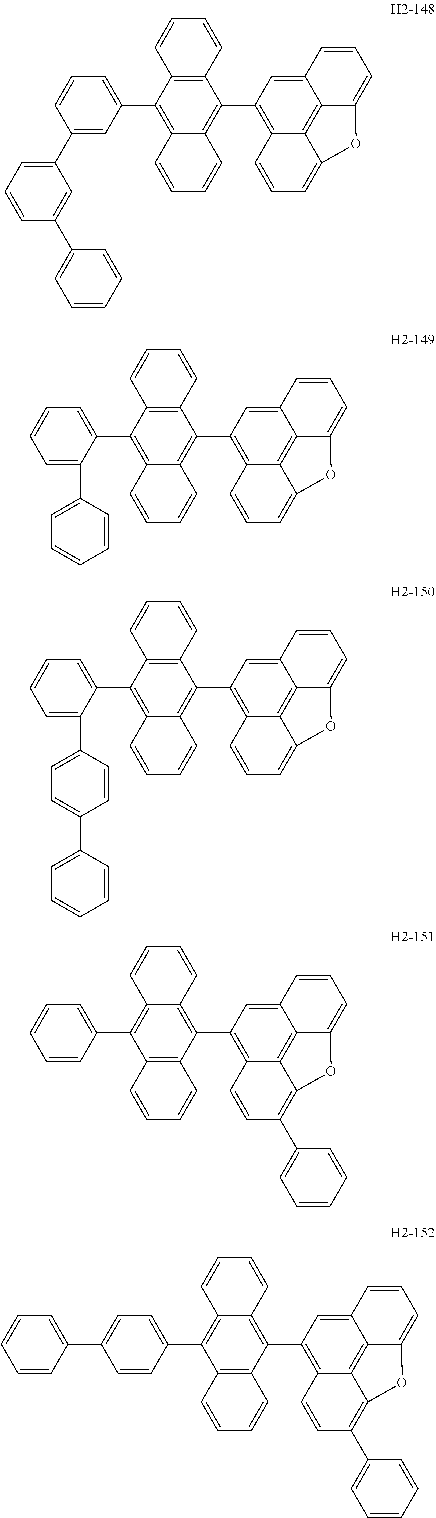 [SMILES: c1ccc(-c2cccc(-c3cccc(-c4c5ccccc5c(-c5cc6cccc7oc8cccc5c8c67)c5ccccc45)c3)c2)cc1 ; c1ccc(-c2ccccc2-c2c3ccccc3c(-c3cc4cccc5oc6cccc3c6c45)c3ccccc23)cc1 ; c1ccc(-c2ccc(-c3ccccc3-c3c4ccccc4c(-c4cc5cccc6oc7cccc4c7c56)c4ccccc34)cc2)cc1 ; c1ccc(-c2c3ccccc3c(-c3cc4cccc5oc6c(-c7ccccc7)ccc3c6c45)c3ccccc23)cc1 ; c1ccc(-c2ccc(-c3c4ccccc4c(-c4cc5cccc6oc7c(-c8ccccc8)ccc4c7c56)c4ccccc34)cc2)cc1] 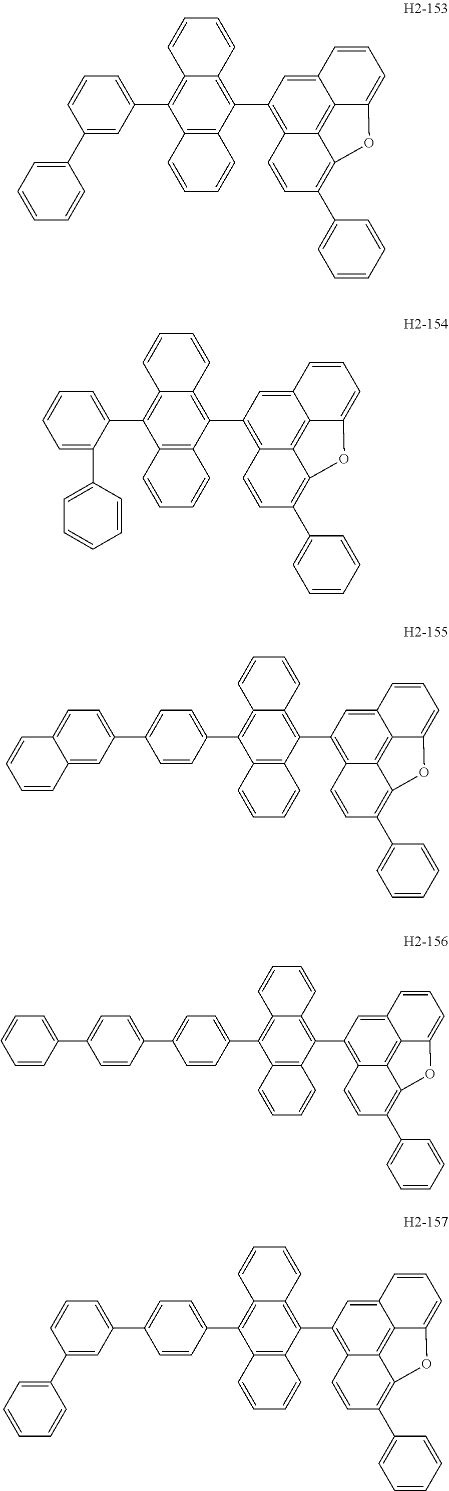 [SMILES: c1ccc(-c2cccc(-c3c4ccccc4c(-c4cc5cccc6oc7c(-c8ccccc8)ccc4c7c56)c4ccccc34)c2)cc1 ; c1ccc(-c2ccccc2-c2c3ccccc3c(-c3cc4cccc5oc6c(-c7ccccc7)ccc3c6c45)c3ccccc23)cc1 ; c1ccc(-c2ccc3c(-c4c5ccccc5c(-c5ccc(-c6ccc7ccccc7c6)cc5)c5ccccc45)cc4cccc5oc2c3c45)cc1 ; c1ccc(-c2ccc(-c3ccc(-c4c5ccccc5c(-c5cc6cccc7oc8c(-c9ccccc9)ccc5c8c67)c5ccccc45)cc3)cc2)cc1 ; c1ccc(-c2cccc(-c3ccc(-c4c5ccccc5c(-c5cc6cccc7oc8c(-c9ccccc9)ccc5c8c67)c5ccccc45)cc3)c2)cc1] 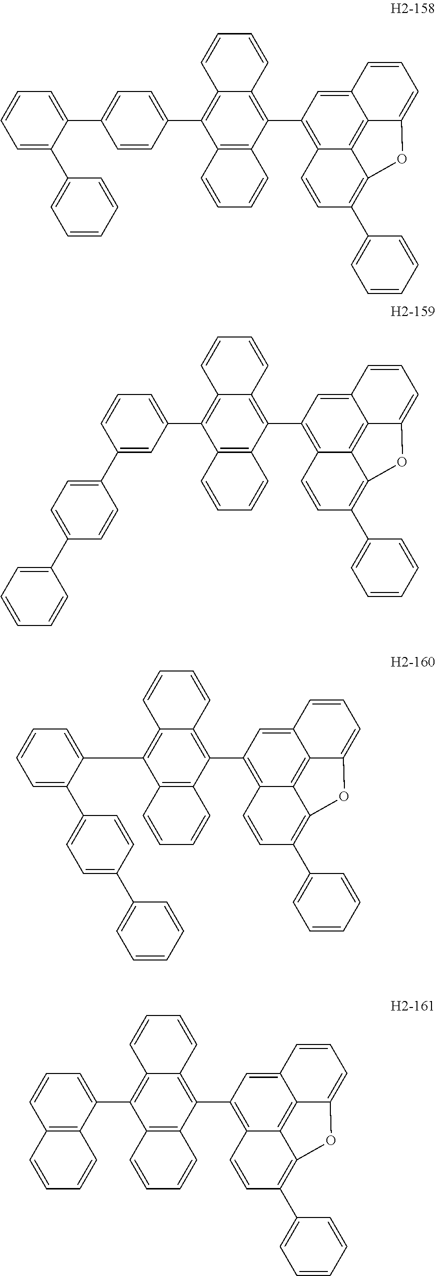 [SMILES: c1ccc(-c2ccccc2-c2ccc(-c3c4ccccc4c(-c4cc5cccc6oc7c(-c8ccccc8)ccc4c7c56)c4ccccc34)cc2)cc1 ; c1ccc(-c2ccc(-c3cccc(-c4c5ccccc5c(-c5cc6cccc7oc8c(-c9ccccc9)ccc5c8c67)c5ccccc45)c3)cc2)cc1 ; c1ccc(-c2ccc(-c3ccccc3-c3c4ccccc4c(-c4cc5cccc6oc7c(-c8ccccc8)ccc4c7c56)c4ccccc34)cc2)cc1 ; c1ccc(-c2ccc3c(-c4c5ccccc5c(-c5cccc6ccccc56)c5ccccc45)cc4cccc5oc2c3c45)cc1] 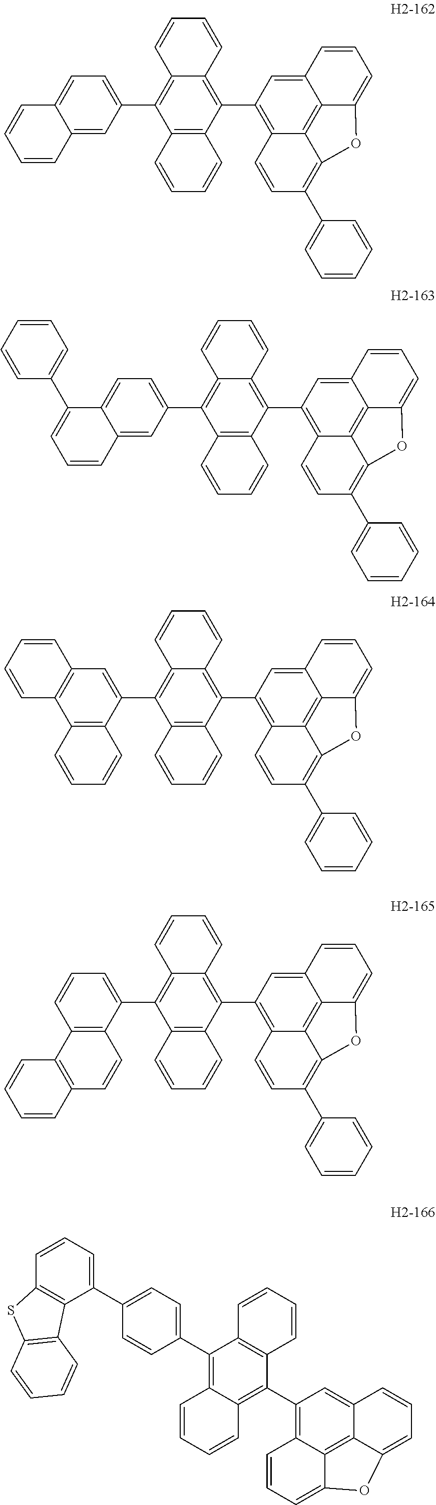 [SMILES: c1ccc(-c2ccc3c(-c4c5ccccc5c(-c5ccc6ccccc6c5)c5ccccc45)cc4cccc5oc2c3c45)cc1 ; c1ccc(-c2cccc3cc(-c4c5ccccc5c(-c5cc6cccc7oc8c(-c9ccccc9)ccc5c8c67)c5ccccc45)ccc23)cc1 ; c1ccc(-c2ccc3c(-c4c5ccccc5c(-c5cc6ccccc6c6ccccc56)c5ccccc45)cc4cccc5oc2c3c45)cc1 ; c1ccc(-c2ccc3c(-c4c5ccccc5c(-c5cccc6c5ccc5ccccc56)c5ccccc45)cc4cccc5oc2c3c45)cc1 ; c1cc2cc(-c3c4ccccc4c(-c4ccc(-c5cccc6sc7ccccc7c56)cc4)c4ccccc34)c3cccc4oc(c1)c2c43] 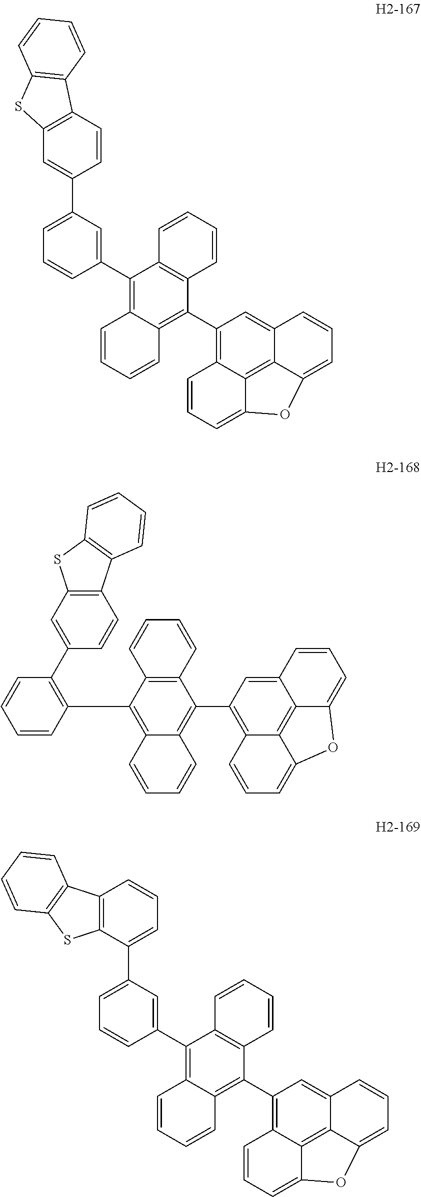 [SMILES: c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1 ; c1cc(-c2c3ccccc3c(-c3cc4cccc5oc6cccc3c6c45)c3ccccc23)cc(-c2cccc3c2sc2ccccc23)c1] 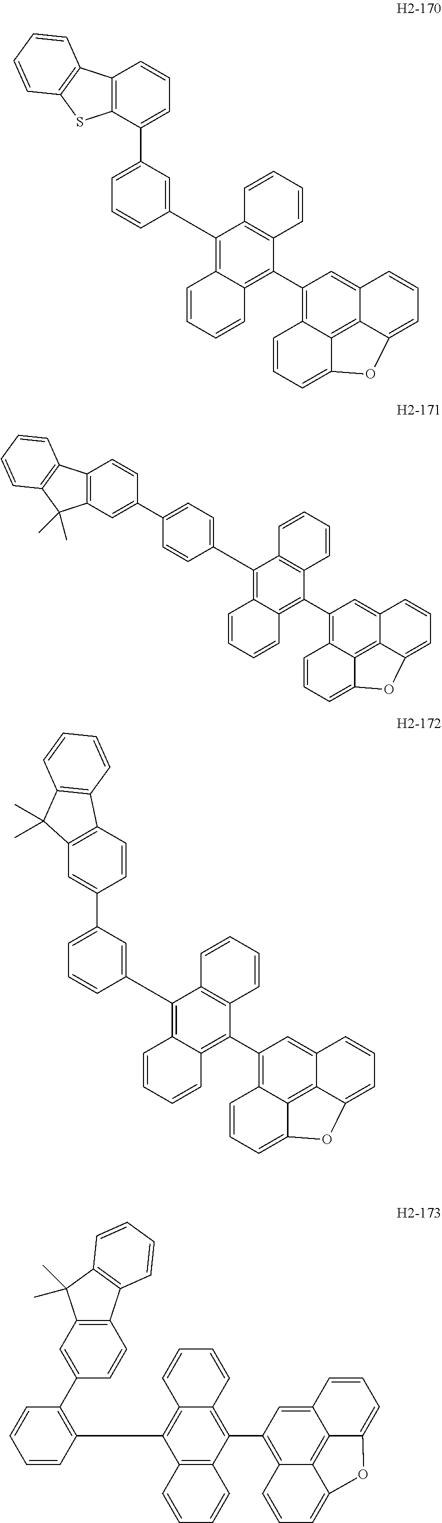 [SMILES: c1cc(-c2c3ccccc3c(-c3cc4cccc5oc6cccc3c6c45)c3ccccc23)cc(-c2cccc3c2sc2ccccc23)c1 ; CC1(C)c2ccccc2-c2ccc(-c3ccc(-c4c5ccccc5c(-c5cc6cccc7oc8cccc5c8c67)c5ccccc45)cc3)cc21 ; CC1(C)c2ccccc2-c2ccc(-c3cccc(-c4c5ccccc5c(-c5cc6cccc7oc8cccc5c8c67)c5ccccc45)c3)cc21 ; CC1(C)c2ccccc2-c2ccc(-c3ccccc3-c3c4ccccc4c(-c4cc5cccc6oc7cccc4c7c56)c4ccccc34)cc21] 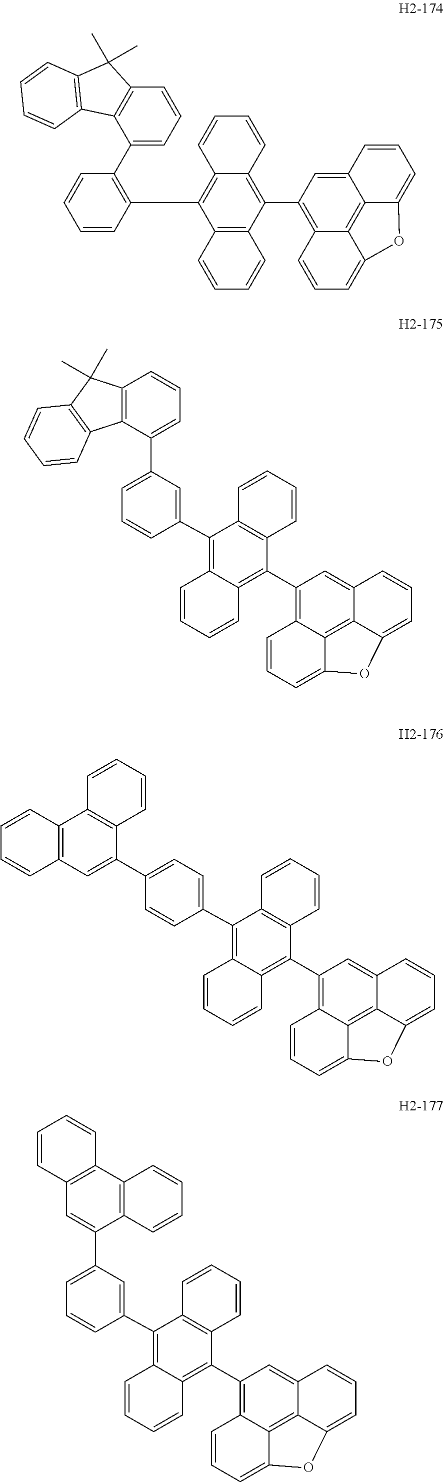 [SMILES: CC1(C)c2ccccc2-c2c(-c3ccccc3-c3c4ccccc4c(-c4cc5cccc6oc7cccc4c7c56)c4ccccc34)cccc21 ; CC1(C)c2ccccc2-c2c(-c3cccc(-c4c5ccccc5c(-c5cc6cccc7oc8cccc5c8c67)c5ccccc45)c3)cccc21 ; c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1] 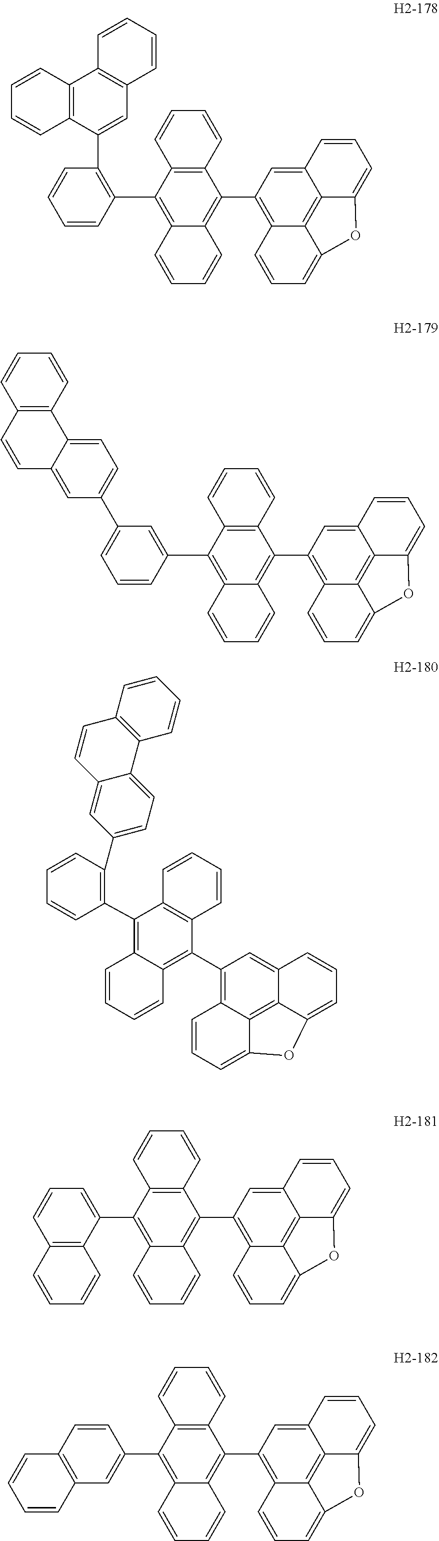 [SMILES: c1ccc(-c2cc3ccccc3c3ccccc23)c(-c2c3ccccc3c(-c3cc4cccc5oc6cccc3c6c45)c3ccccc23)c1 ; c1cc(-c2ccc3c(ccc4ccccc43)c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(ccc4ccccc43)c2)c1 ; c1ccc2c(-c3c4ccccc4c(-c4cc5cccc6oc7cccc4c7c56)c4ccccc34)cccc2c1 ; c1ccc2cc(-c3c4ccccc4c(-c4cc5cccc6oc7cccc4c7c56)c4ccccc34)ccc2c1] 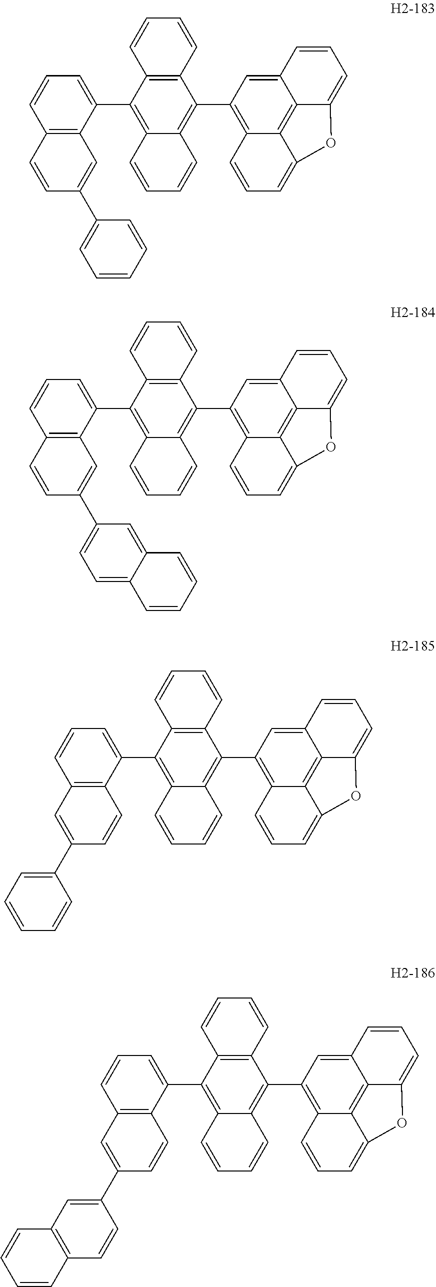 [SMILES: c1ccc(-c2ccc3cccc(-c4c5ccccc5c(-c5cc6cccc7oc8cccc5c8c67)c5ccccc45)c3c2)cc1 ; c1ccc2cc(-c3ccc4cccc(-c5c6ccccc6c(-c6cc7cccc8oc9cccc6c9c78)c6ccccc56)c4c3)ccc2c1 ; c1ccc(-c2ccc3c(-c4c5ccccc5c(-c5cc6cccc7oc8cccc5c8c67)c5ccccc45)cccc3c2)cc1 ; c1ccc2cc(-c3ccc4c(-c5c6ccccc6c(-c6cc7cccc8oc9cccc6c9c78)c6ccccc56)cccc4c3)ccc2c1] 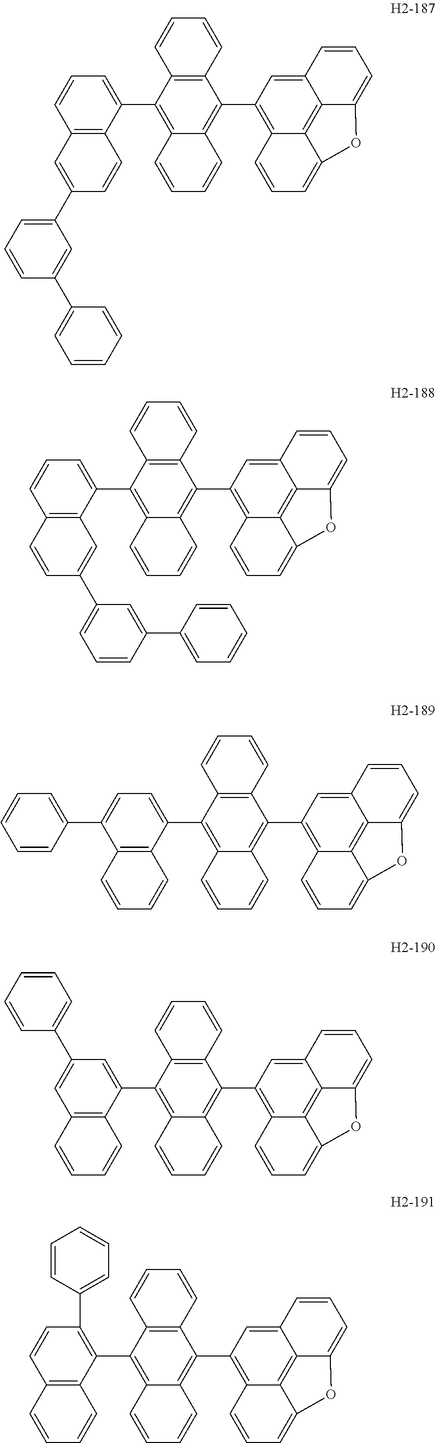 [SMILES: c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1 ; c1ccc(-c2ccc(-c3c4ccccc4c(-c4cc5cccc6oc7cccc4c7c56)c4ccccc34)c3ccccc23)cc1 ; c1ccc(-c2cc(-c3c4ccccc4c(-c4cc5cccc6oc7cccc4c7c56)c4ccccc34)c3ccccc3c2)cc1 ; c1ccc(-c2ccc3ccccc3c2-c2c3ccccc3c(-c3cc4cccc5oc6cccc3c6c45)c3ccccc23)cc1] 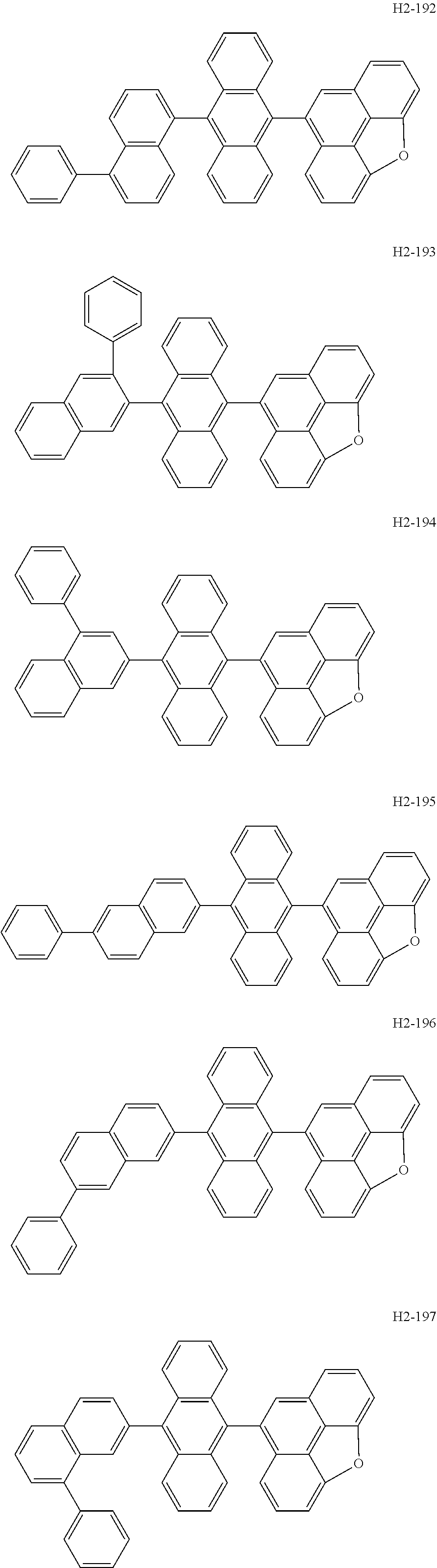 [SMILES: c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)cc3ccccc23)cc1 ; c1ccc(-c2ccc3cc(-c4c5ccccc5c(-c5cc6cccc7oc8cccc5c8c67)c5ccccc45)ccc3c2)cc1 ; c1ccc(-c2ccc3ccc(-c4c5ccccc5c(-c5cc6cccc7oc8cccc5c8c67)c5ccccc45)cc3c2)cc1 ; c1ccc(-c2cccc3ccc(-c4c5ccccc5c(-c5cc6cccc7oc8cccc5c8c67)c5ccccc45)cc23)cc1] 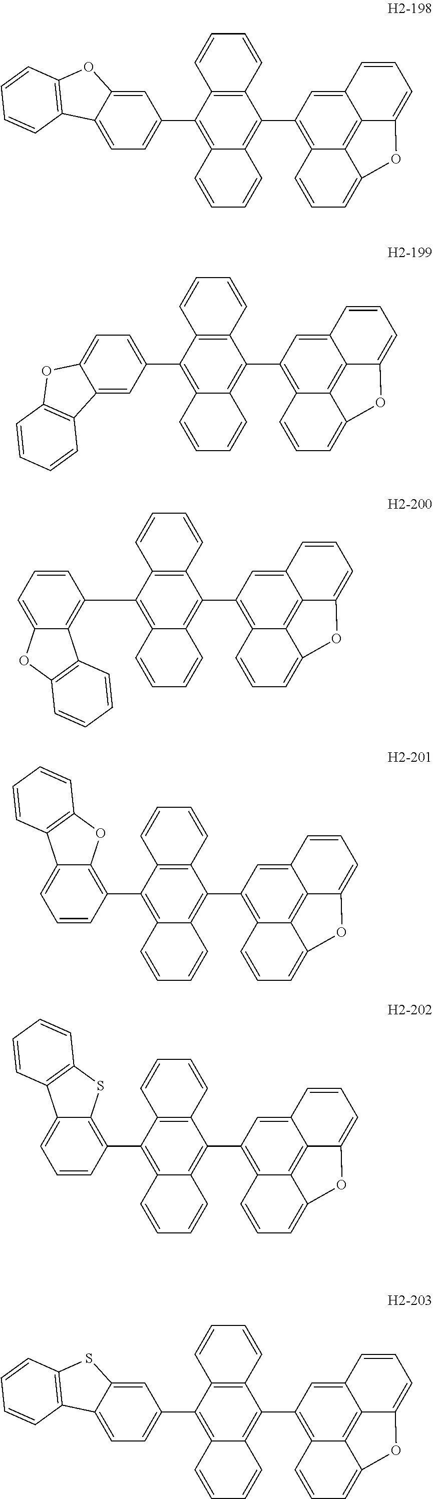 [SMILES: c1ccc2c(c1)oc1cc(-c3c4ccccc4c(-c4cc5cccc6oc7cccc4c7c56)c4ccccc34)ccc12 ; c1ccc2c(c1)oc1ccc(-c3c4ccccc4c(-c4cc5cccc6oc7cccc4c7c56)c4ccccc34)cc12 ; c1ccc2c(c1)oc1cccc(-c3c4ccccc4c(-c4cc5cccc6oc7cccc4c7c56)c4ccccc34)c12 ; c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43] 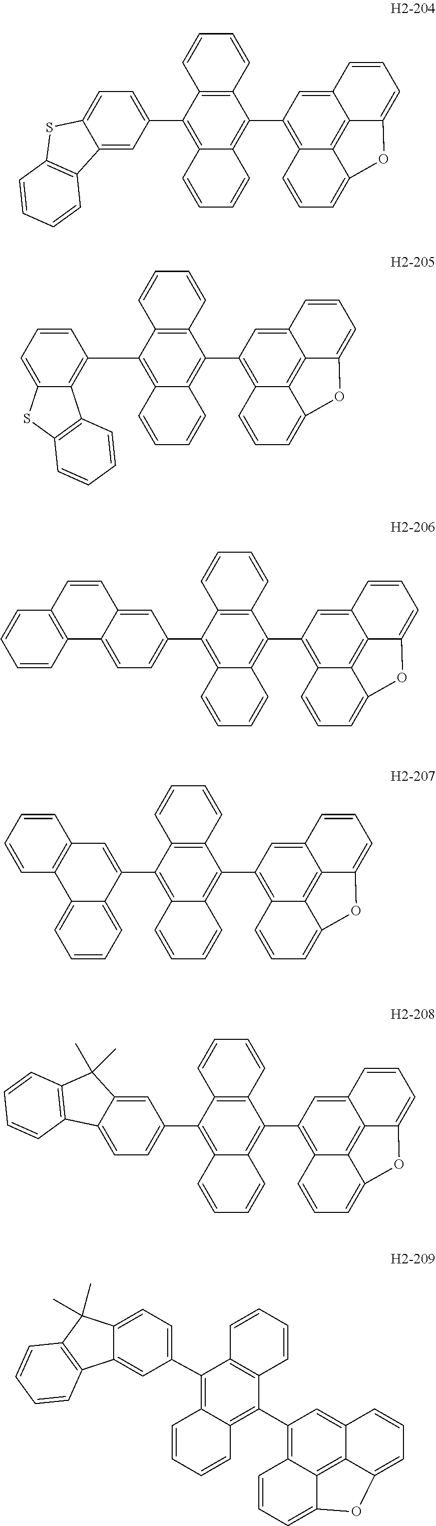 [SMILES: c1cc2cc(-c3c4ccccc4c(-c4ccc5sc6ccccc6c5c4)c4ccccc34)c3cccc4oc(c1)c2c43 ; c1cc2cc(-c3c4ccccc4c(-c4cccc5sc6ccccc6c45)c4ccccc34)c3cccc4oc(c1)c2c43 ; c1ccc2c(c1)ccc1cc(-c3c4ccccc4c(-c4cc5cccc6oc7cccc4c7c56)c4ccccc34)ccc12 ; c1ccc2c(c1)cc(-c1c3ccccc3c(-c3cc4cccc5oc6cccc3c6c45)c3ccccc13)c1ccccc12 ; CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21] 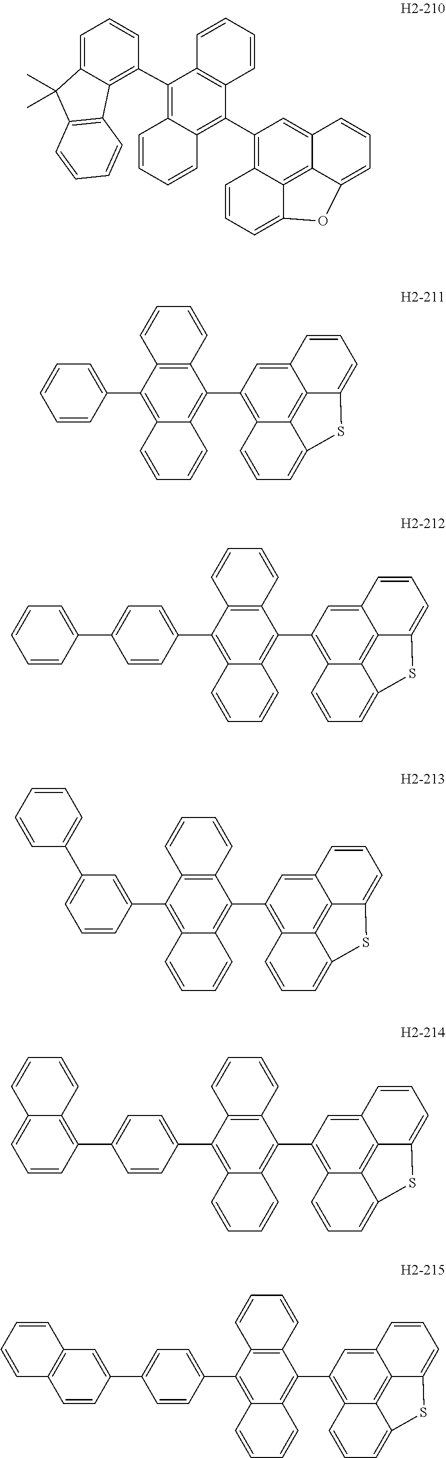 [SMILES: CC1(C)c2ccccc2-c2c(-c3c4ccccc4c(-c4cc5cccc6oc7cccc4c7c56)c4ccccc34)cccc21 ; c1ccc(-c2c3ccccc3c(-c3cc4cccc5sc6cccc3c6c45)c3ccccc23)cc1 ; c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1 ; c1ccc2c(-c3ccc(-c4c5ccccc5c(-c5cc6cccc7sc8cccc5c8c67)c5ccccc45)cc3)cccc2c1 ; c1ccc2cc(-c3ccc(-c4c5ccccc5c(-c5cc6cccc7sc8cccc5c8c67)c5ccccc45)cc3)ccc2c1] 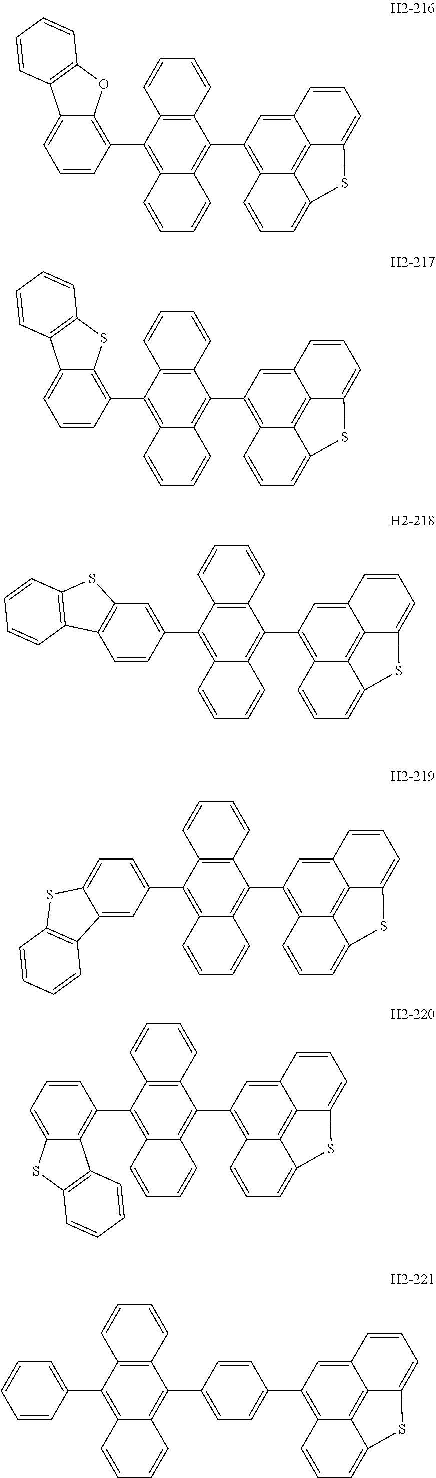 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12 ; c1ccc(-c2c3ccccc3c(-c3ccc(-c4cc5cccc6sc7cccc4c7c56)cc3)c3ccccc23)cc1] 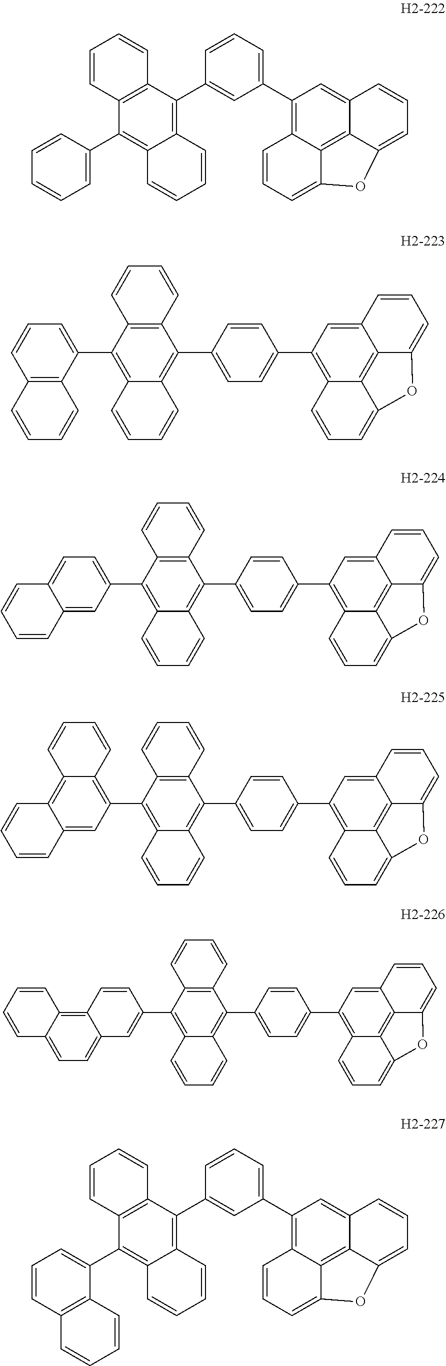 [SMILES: c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1 ; c1ccc2cc(-c3c4ccccc4c(-c4ccc(-c5cc6cccc7oc8cccc5c8c67)cc4)c4ccccc34)ccc2c1 ; c1ccc2c(c1)cc(-c1c3ccccc3c(-c3ccc(-c4cc5cccc6oc7cccc4c7c56)cc3)c3ccccc13)c1ccccc12 ; c1ccc2c(c1)ccc1cc(-c3c4ccccc4c(-c4ccc(-c5cc6cccc7oc8cccc5c8c67)cc4)c4ccccc34)ccc12 ; c1cc(-c2c3ccccc3c(-c3cccc4ccccc34)c3ccccc23)cc(-c2cc3cccc4oc5cccc2c5c34)c1] 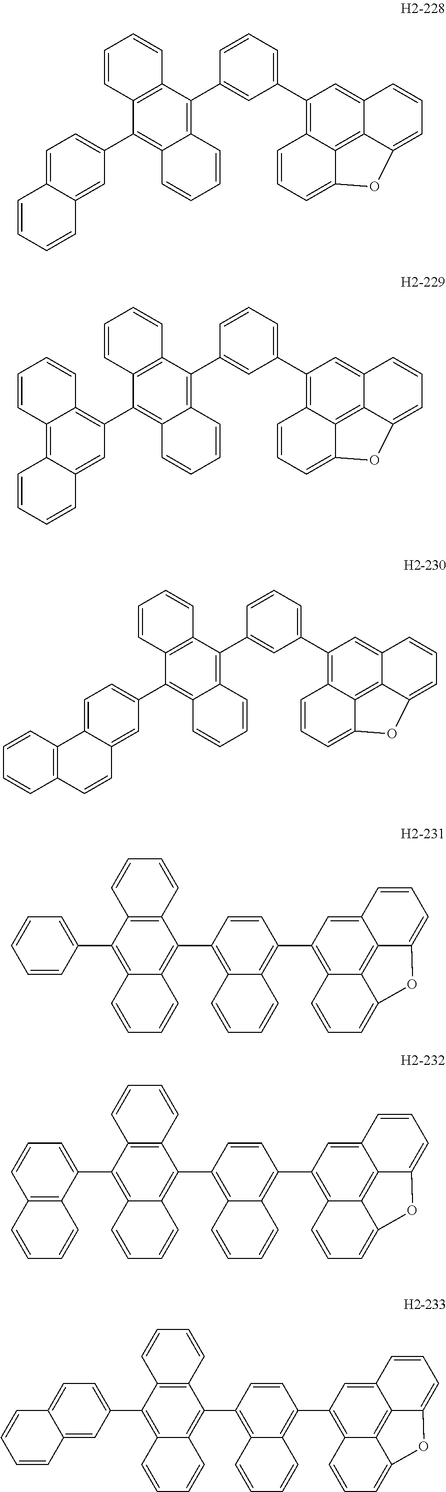 [SMILES: c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1 ; c1cc(-c2c3ccccc3c(-c3ccc4c(ccc5ccccc54)c3)c3ccccc23)cc(-c2cc3cccc4oc5cccc2c5c34)c1 ; c1ccc(-c2c3ccccc3c(-c3ccc(-c4cc5cccc6oc7cccc4c7c56)c4ccccc34)c3ccccc23)cc1 ; c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1] 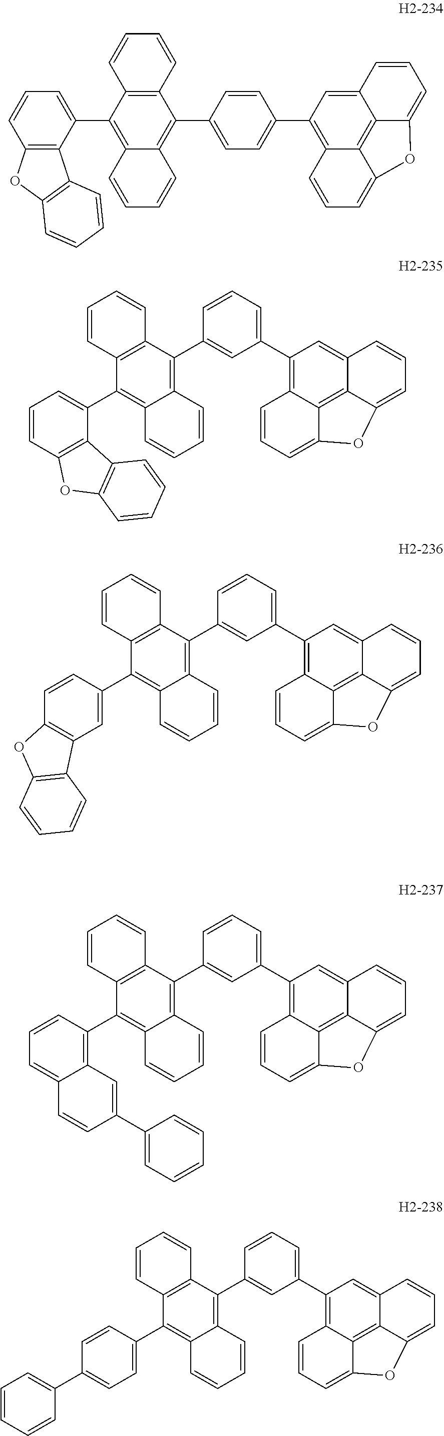 [SMILES: c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1 ; c1cc(-c2c3ccccc3c(-c3ccc4oc5ccccc5c4c3)c3ccccc23)cc(-c2cc3cccc4oc5cccc2c5c34)c1 ; c1ccc(-c2ccc3cccc(-c4c5ccccc5c(-c5cccc(-c6cc7cccc8oc9cccc6c9c78)c5)c5ccccc45)c3c2)cc1 ; c1ccc(-c2ccc(-c3c4ccccc4c(-c4cccc(-c5cc6cccc7oc8cccc5c8c67)c4)c4ccccc34)cc2)cc1] 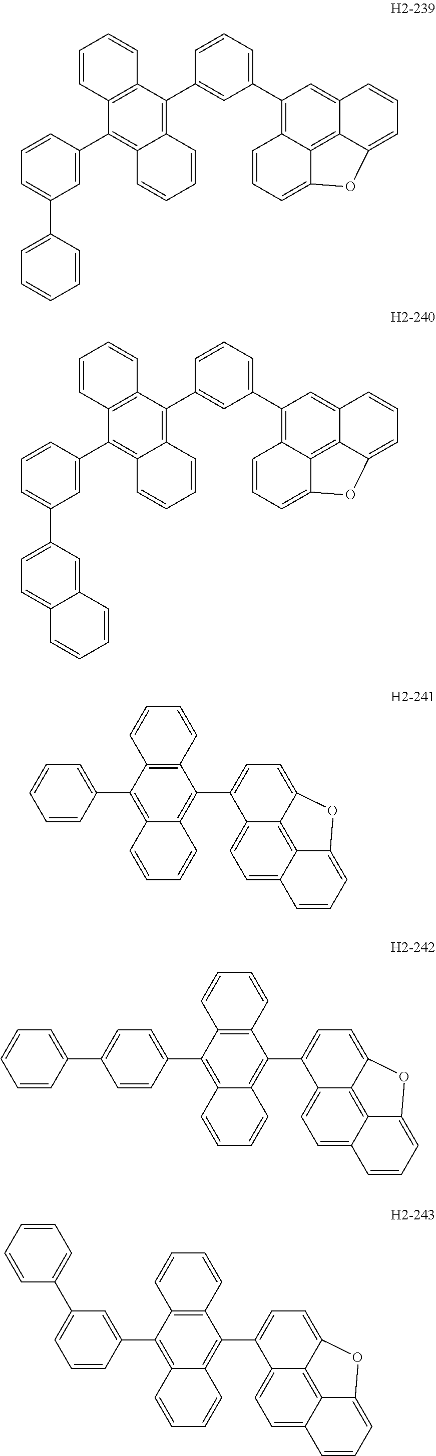 [SMILES: c1ccc(-c2cccc(-c3c4ccccc4c(-c4cccc(-c5cc6cccc7oc8cccc5c8c67)c4)c4ccccc34)c2)cc1 ; c1cc(-c2ccc3ccccc3c2)cc(-c2c3ccccc3c(-c3cccc(-c4cc5cccc6oc7cccc4c7c56)c3)c3ccccc23)c1 ; c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1] 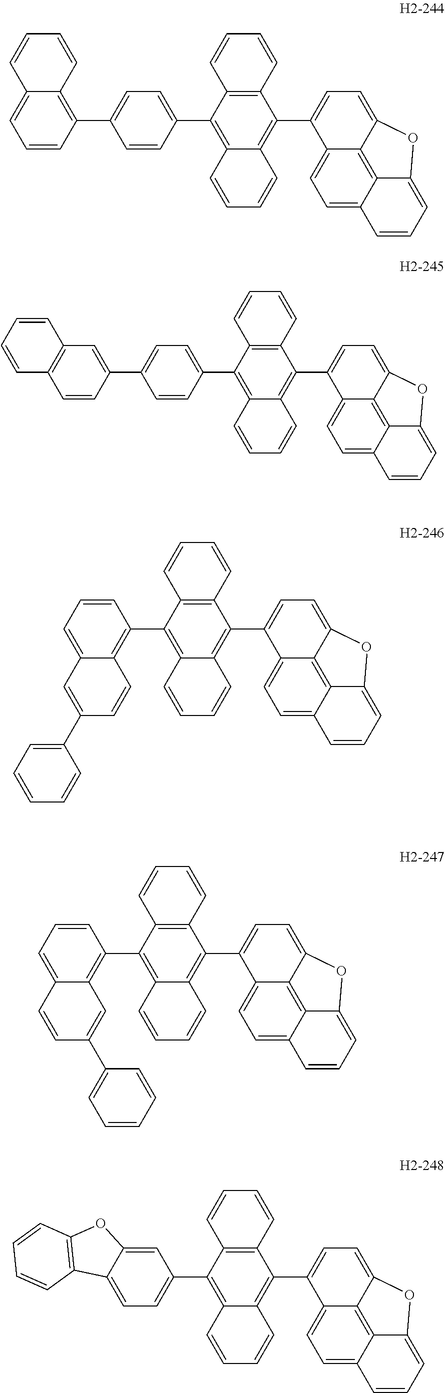 [SMILES: c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1 ; c1ccc(-c2ccc3c(-c4c5ccccc5c(-c5ccc6oc7cccc8ccc5c6c87)c5ccccc45)cccc3c2)cc1 ; c1ccc(-c2ccc3cccc(-c4c5ccccc5c(-c5ccc6oc7cccc8ccc5c6c87)c5ccccc45)c3c2)cc1 ; c1ccc2c(c1)oc1cc(-c3c4ccccc4c(-c4ccc5oc6cccc7ccc4c5c76)c4ccccc34)ccc12] 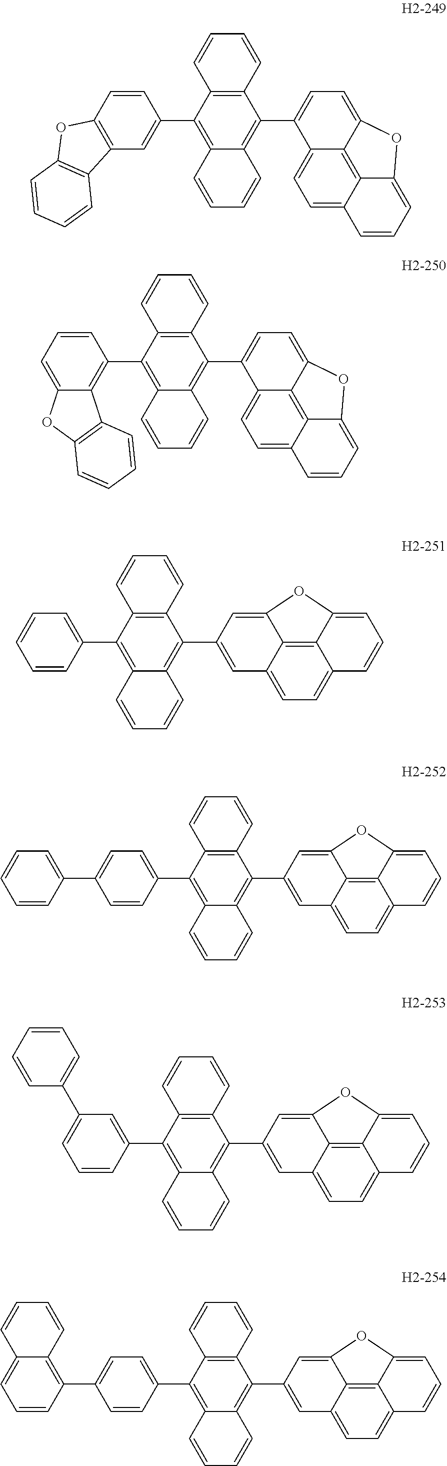 [SMILES: c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12 ; c1ccc(-c2c3ccccc3c(-c3cc4ccc5cccc6oc(c3)c4c56)c3ccccc23)cc1 ; c1ccc(-c2ccc(-c3c4ccccc4c(-c4cc5ccc6cccc7oc(c4)c5c67)c4ccccc34)cc2)cc1 ; c1ccc(-c2cccc(-c3c4ccccc4c(-c4cc5ccc6cccc7oc(c4)c5c67)c4ccccc34)c2)cc1 ; c1ccc2c(-c3ccc(-c4c5ccccc5c(-c5cc6ccc7cccc8oc(c5)c6c78)c5ccccc45)cc3)cccc2c1] 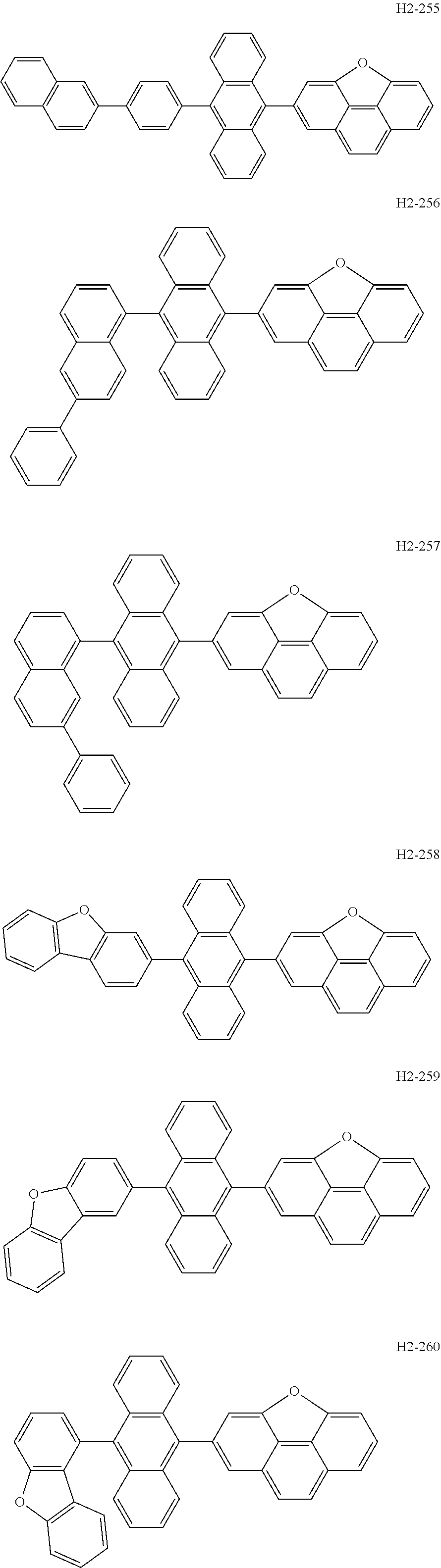 [SMILES: c1ccc2cc(-c3ccc(-c4c5ccccc5c(-c5cc6ccc7cccc8oc(c5)c6c78)c5ccccc45)cc3)ccc2c1 ; c1ccc(-c2ccc3c(-c4c5ccccc5c(-c5cc6ccc7cccc8oc(c5)c6c78)c5ccccc45)cccc3c2)cc1 ; c1ccc(-c2ccc3cccc(-c4c5ccccc5c(-c5cc6ccc7cccc8oc(c5)c6c78)c5ccccc45)c3c2)cc1 ; c1ccc2c(c1)oc1cc(-c3c4ccccc4c(-c4cc5ccc6cccc7oc(c4)c5c67)c4ccccc34)ccc12 ; c1ccc2c(c1)oc1ccc(-c3c4ccccc4c(-c4cc5ccc6cccc7oc(c4)c5c67)c4ccccc34)cc12 ; c1ccc2c(c1)oc1cccc(-c3c4ccccc4c(-c4cc5ccc6cccc7oc(c4)c5c67)c4ccccc34)c12] 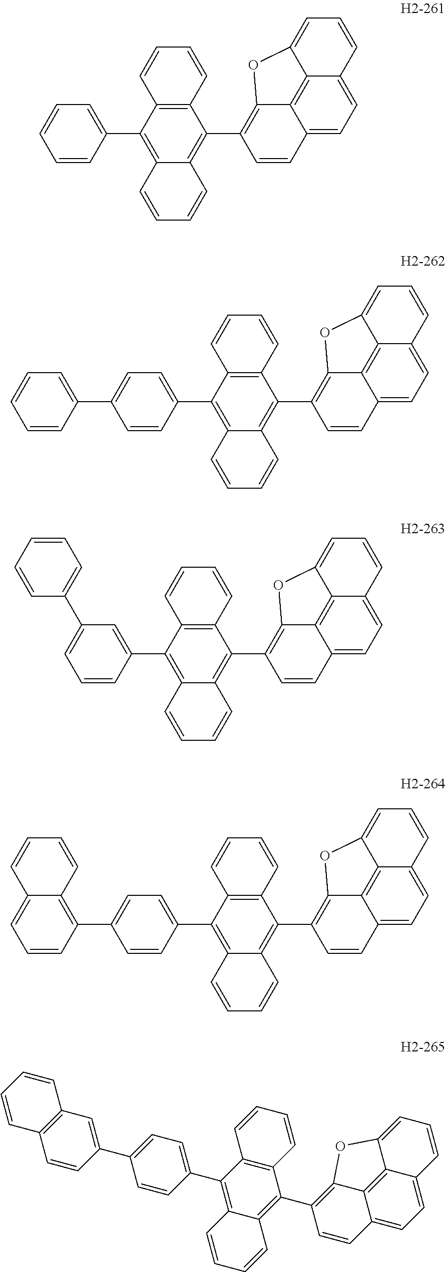 [SMILES: c1ccc(-c2c3ccccc3c(-c3ccc4ccc5cccc6oc3c4c56)c3ccccc23)cc1 ; c1ccc(-c2ccc(-c3c4ccccc4c(-c4ccc5ccc6cccc7oc4c5c67)c4ccccc34)cc2)cc1 ; c1ccc(-c2cccc(-c3c4ccccc4c(-c4ccc5ccc6cccc7oc4c5c67)c4ccccc34)c2)cc1 ; c1ccc2c(-c3ccc(-c4c5ccccc5c(-c5ccc6ccc7cccc8oc5c6c78)c5ccccc45)cc3)cccc2c1 ; c1ccc2cc(-c3ccc(-c4c5ccccc5c(-c5ccc6ccc7cccc8oc5c6c78)c5ccccc45)cc3)ccc2c1] 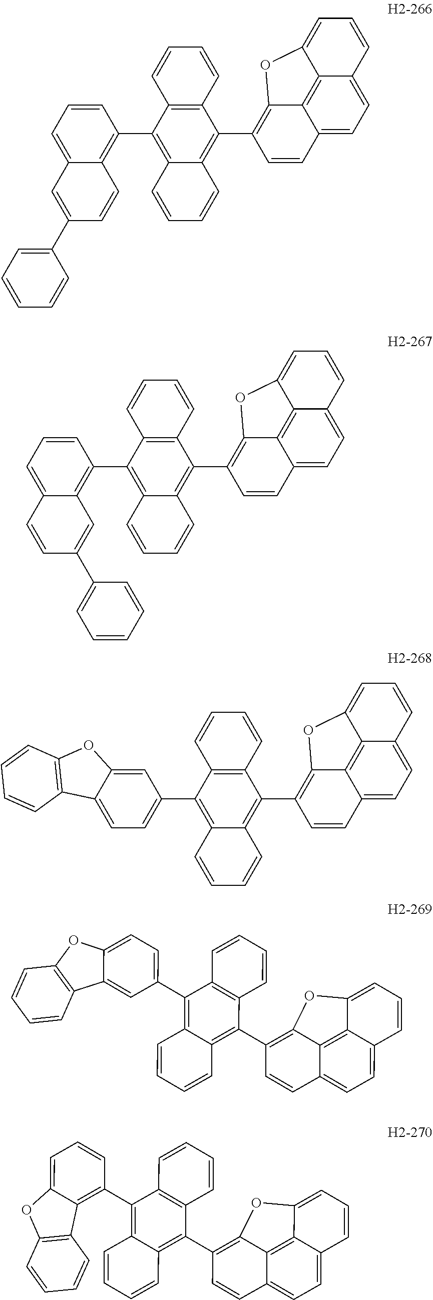 [SMILES: c1ccc(-c2ccc3c(-c4c5ccccc5c(-c5ccc6ccc7cccc8oc5c6c78)c5ccccc45)cccc3c2)cc1 ; c1ccc(-c2ccc3cccc(-c4c5ccccc5c(-c5ccc6ccc7cccc8oc5c6c78)c5ccccc45)c3c2)cc1 ; c1ccc2c(c1)oc1cc(-c3c4ccccc4c(-c4ccc5ccc6cccc7oc4c5c67)c4ccccc34)ccc12 ; c1ccc2c(c1)oc1ccc(-c3c4ccccc4c(-c4ccc5ccc6cccc7oc4c5c67)c4ccccc34)cc12 ; c1ccc2c(c1)oc1cccc(-c3c4ccccc4c(-c4ccc5ccc6cccc7oc4c5c67)c4ccccc34)c12] 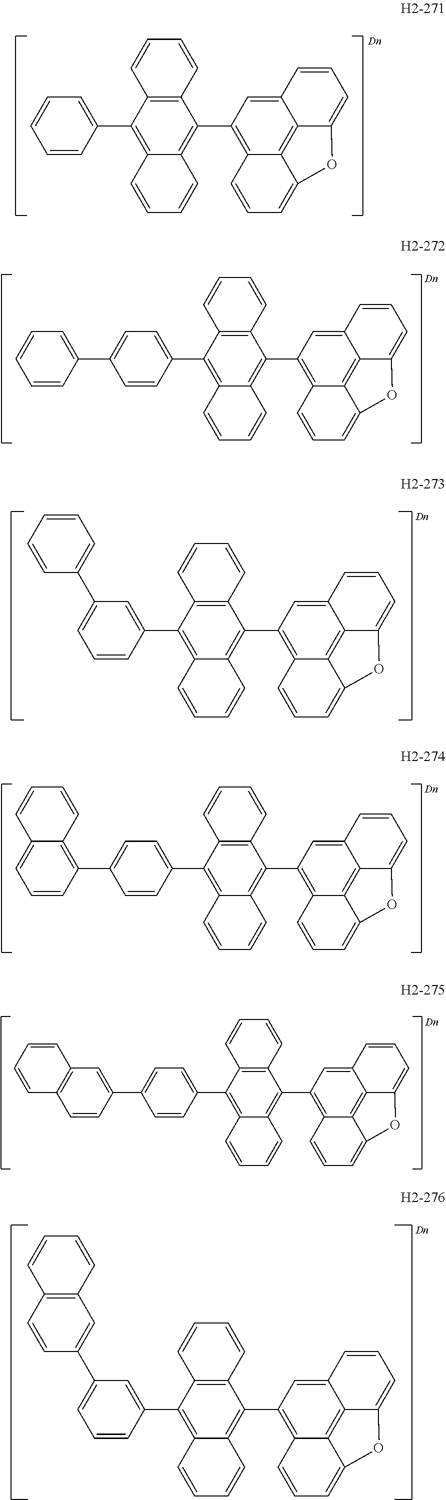 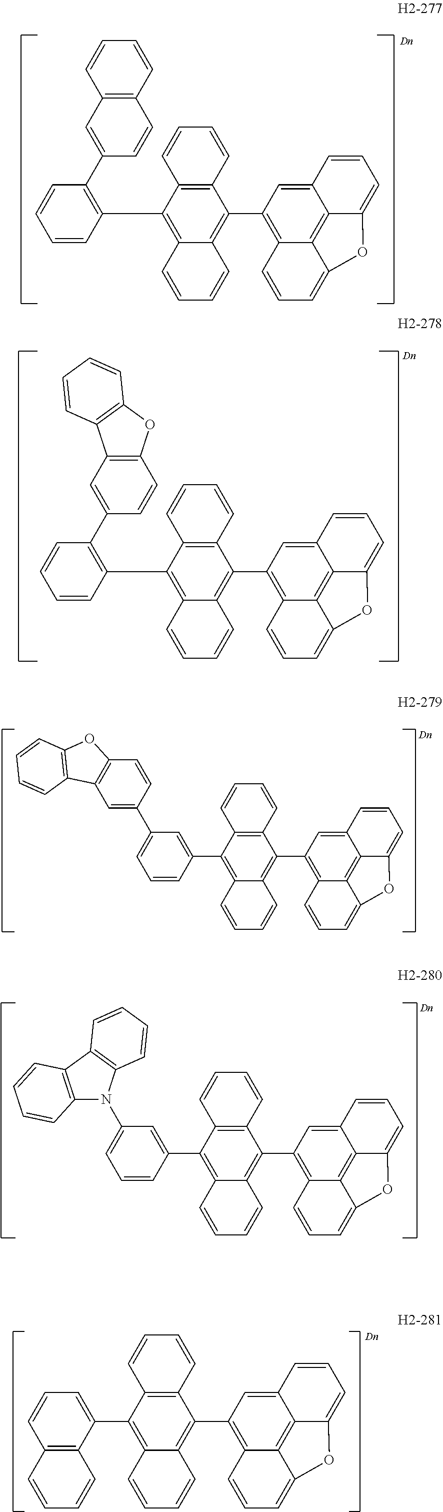 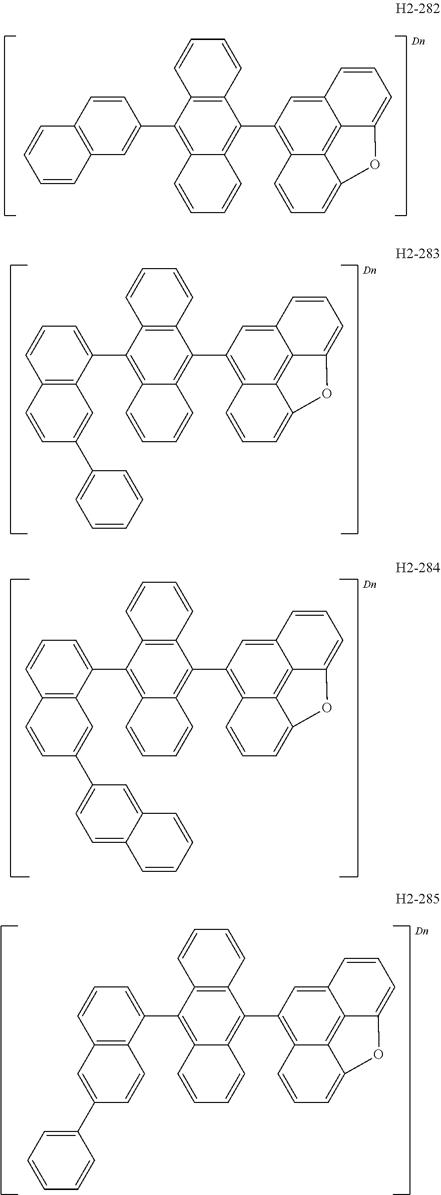 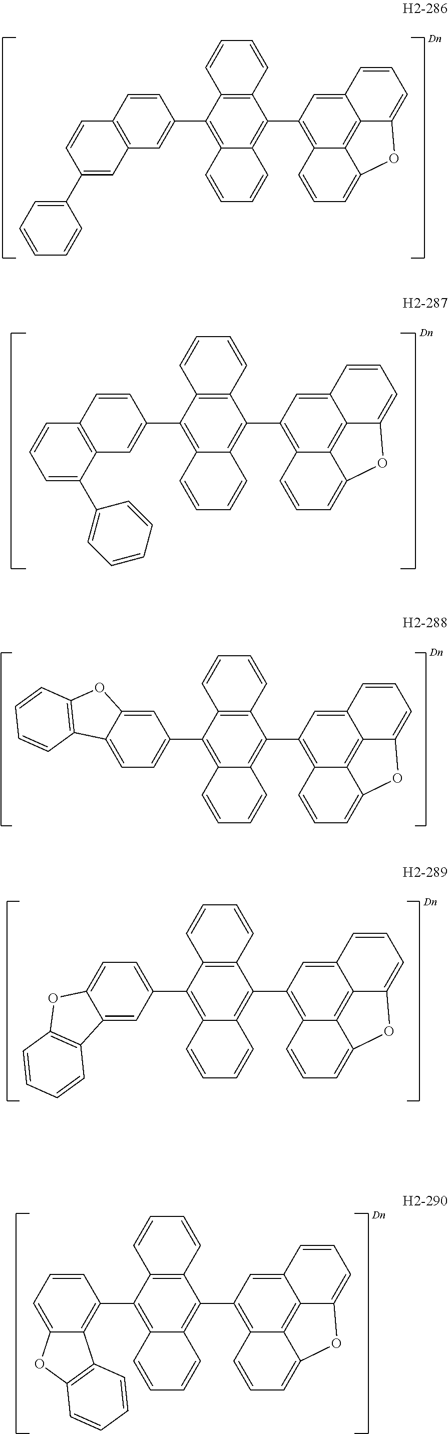 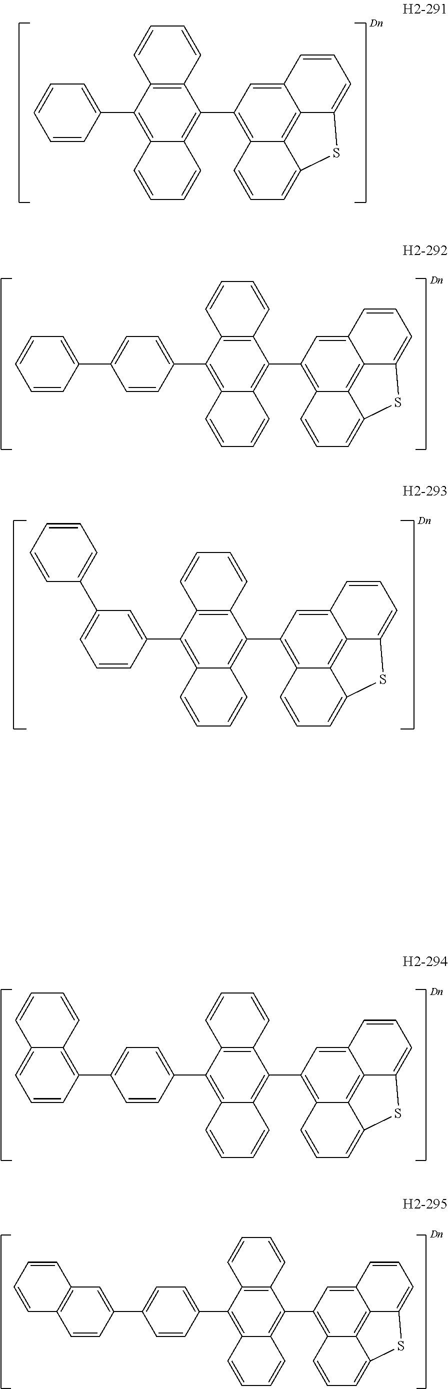 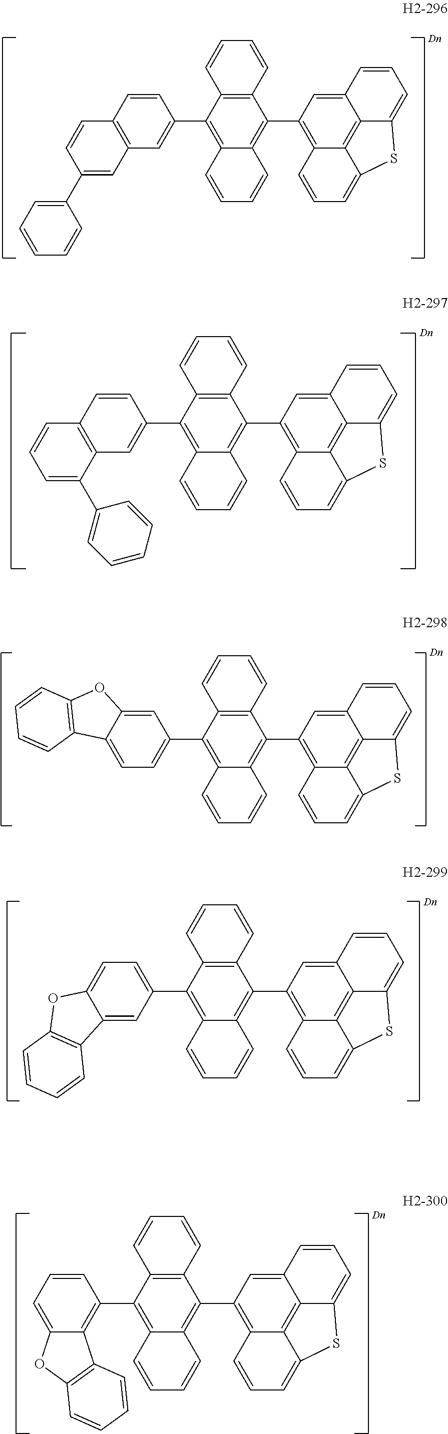 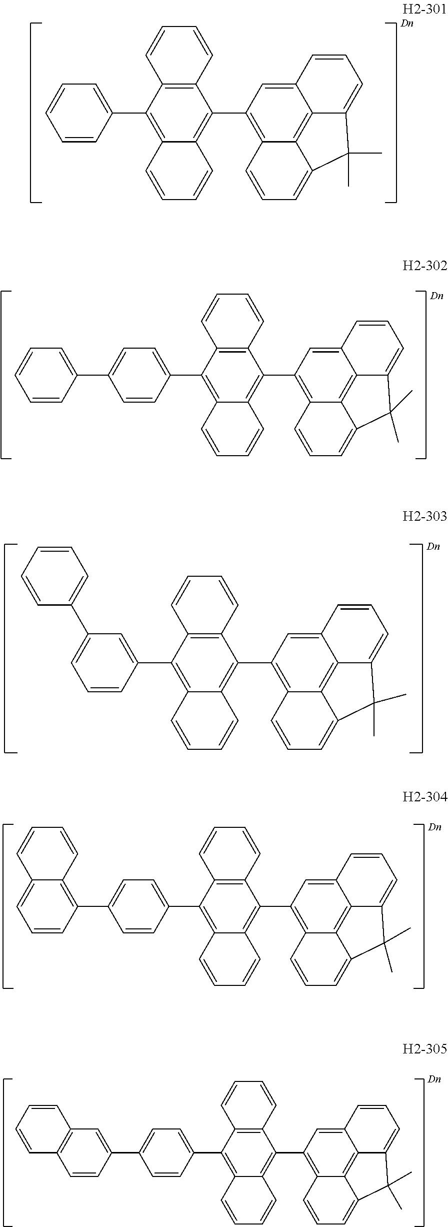 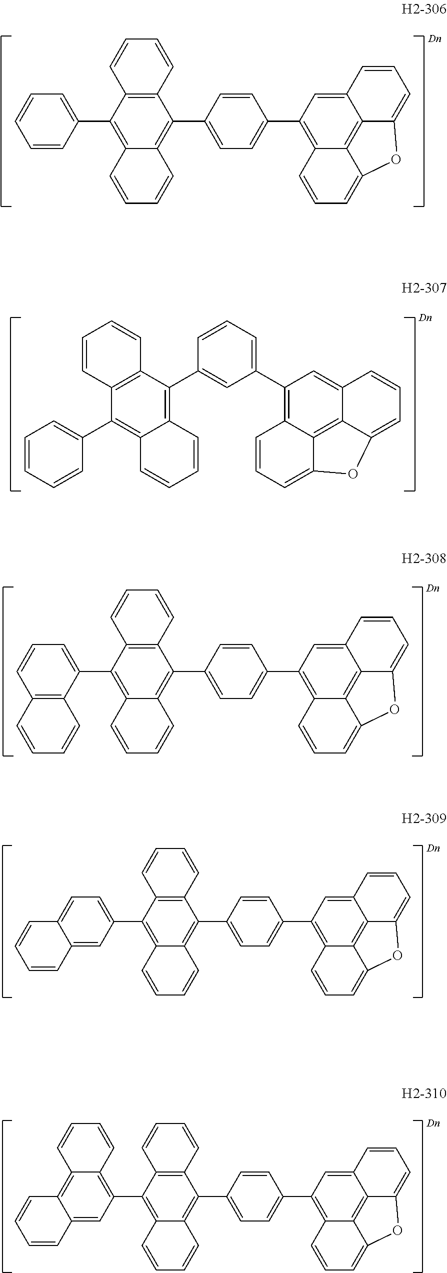 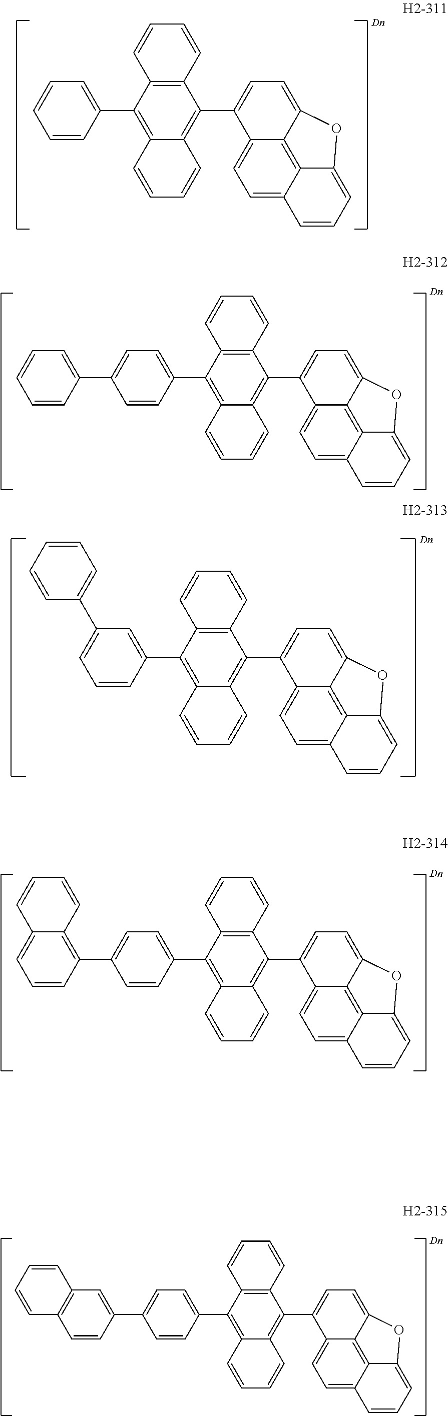 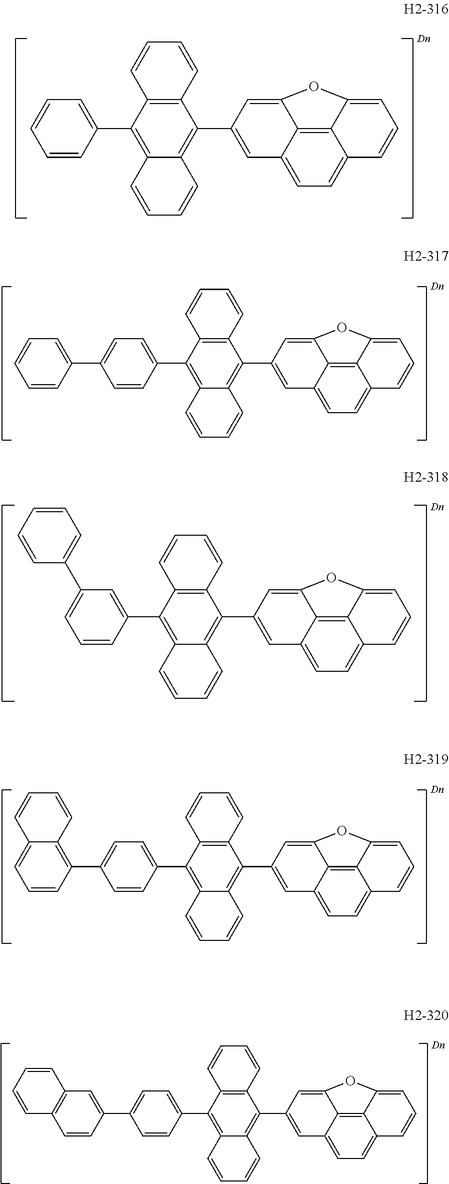 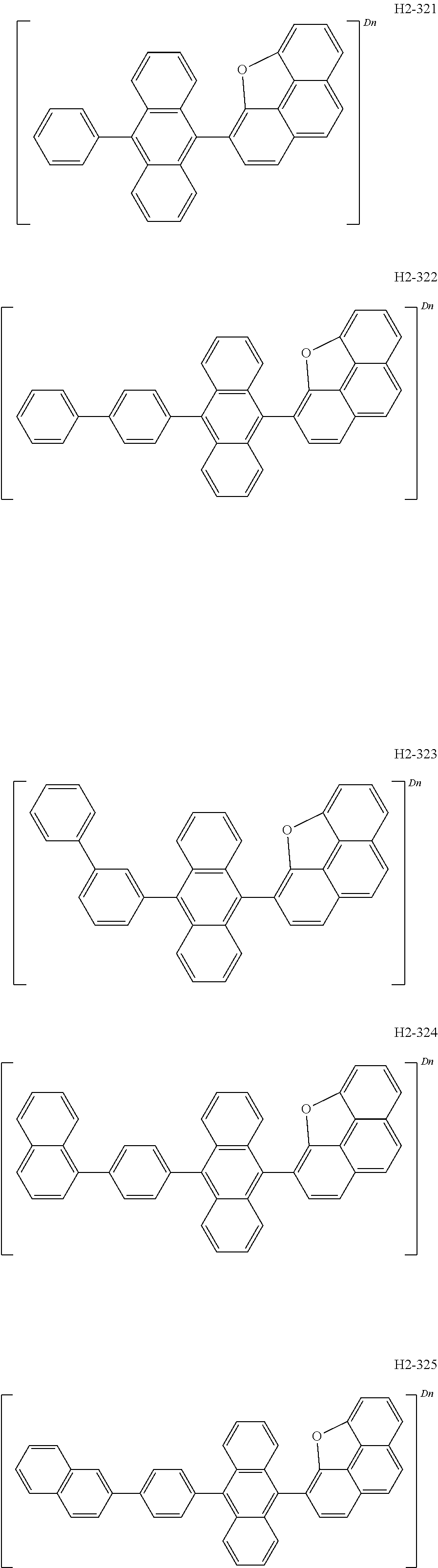 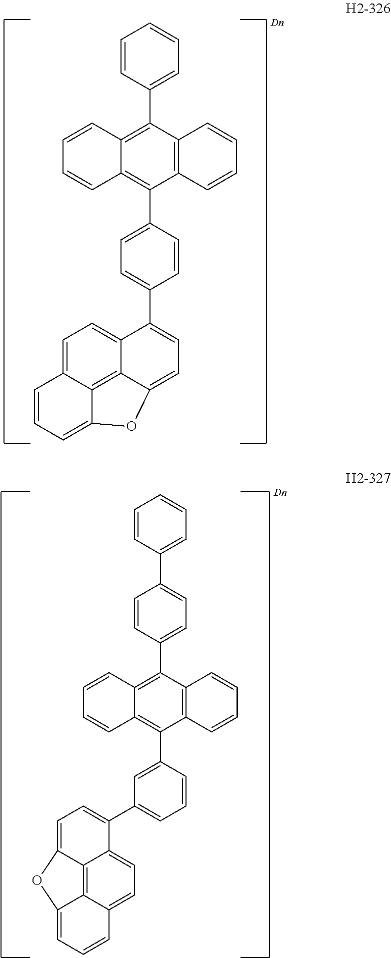 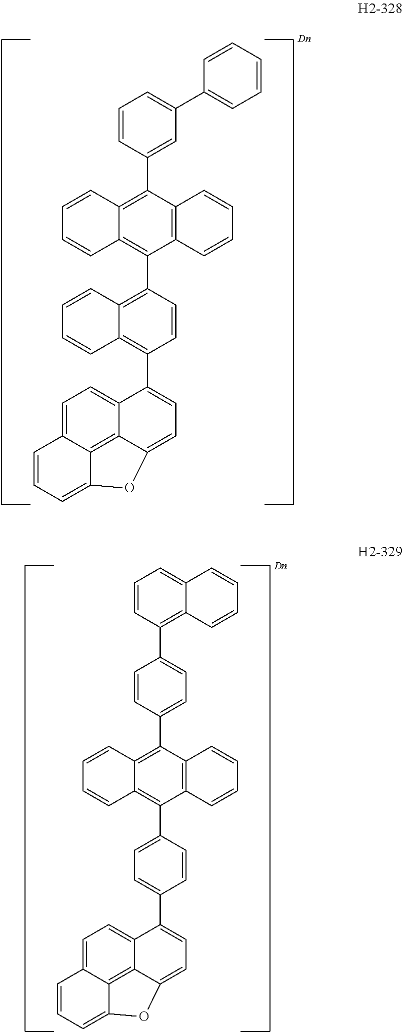 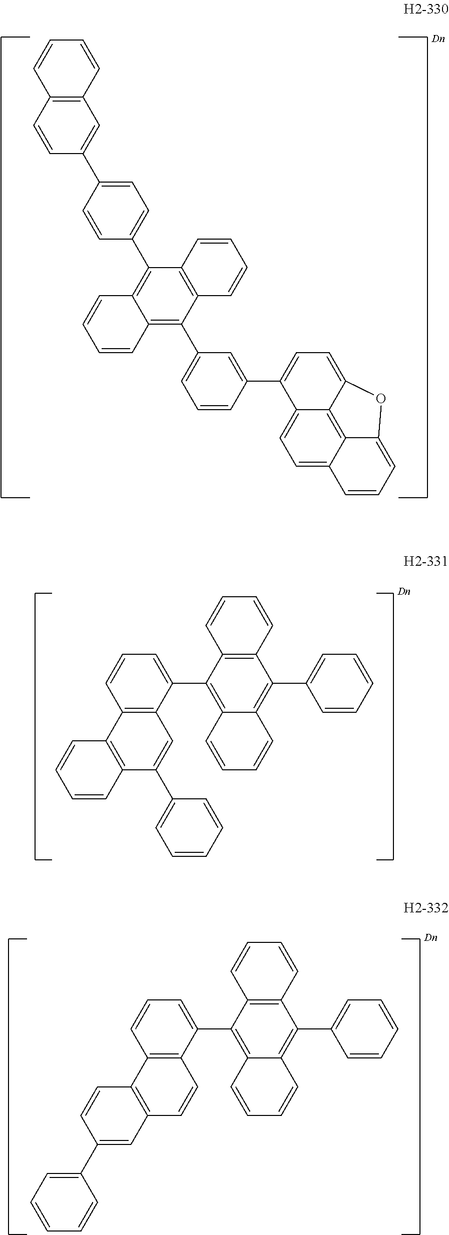 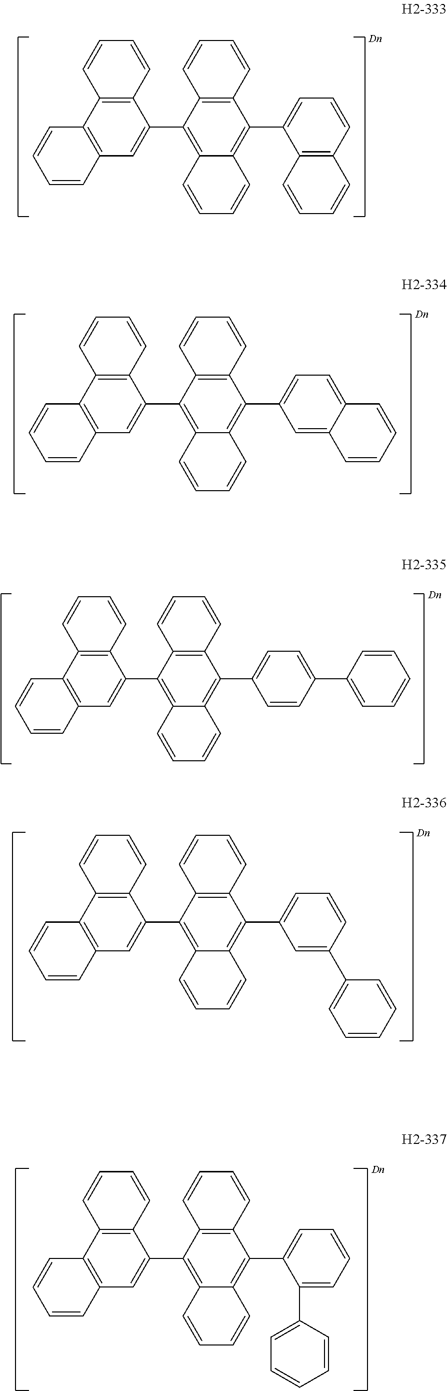 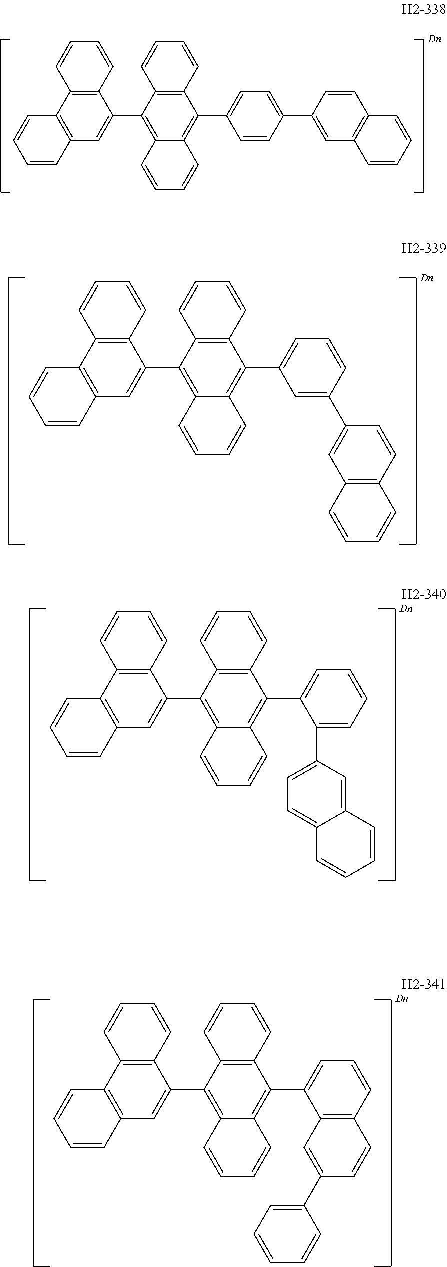 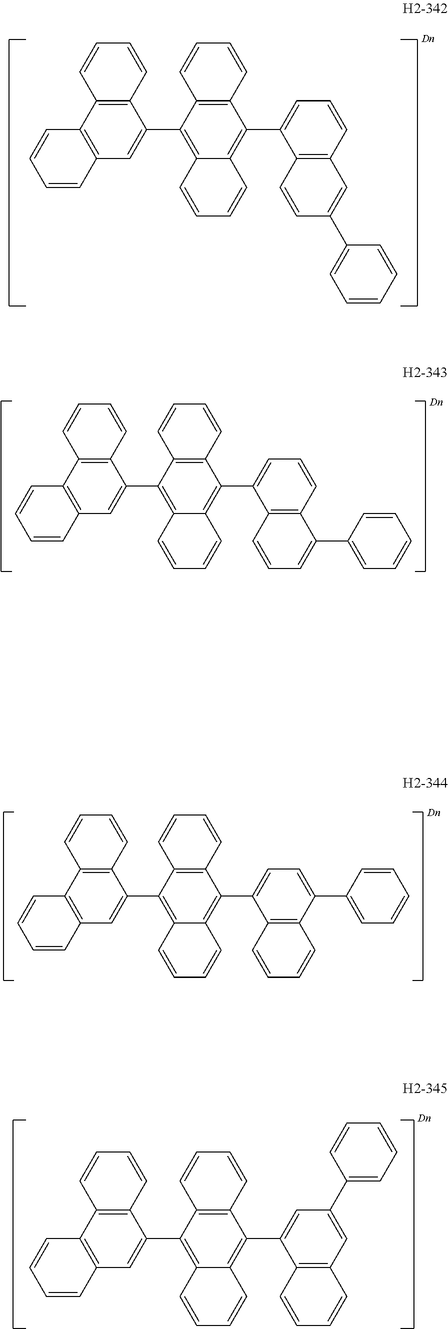 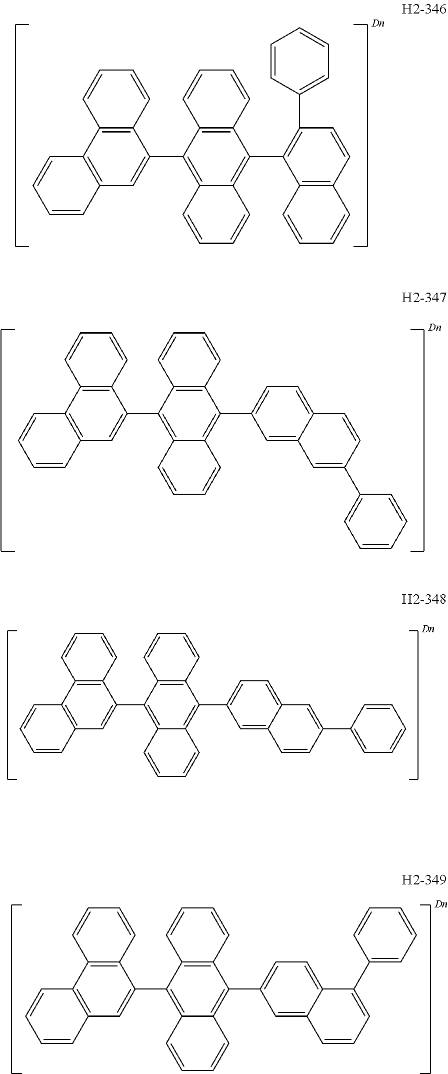 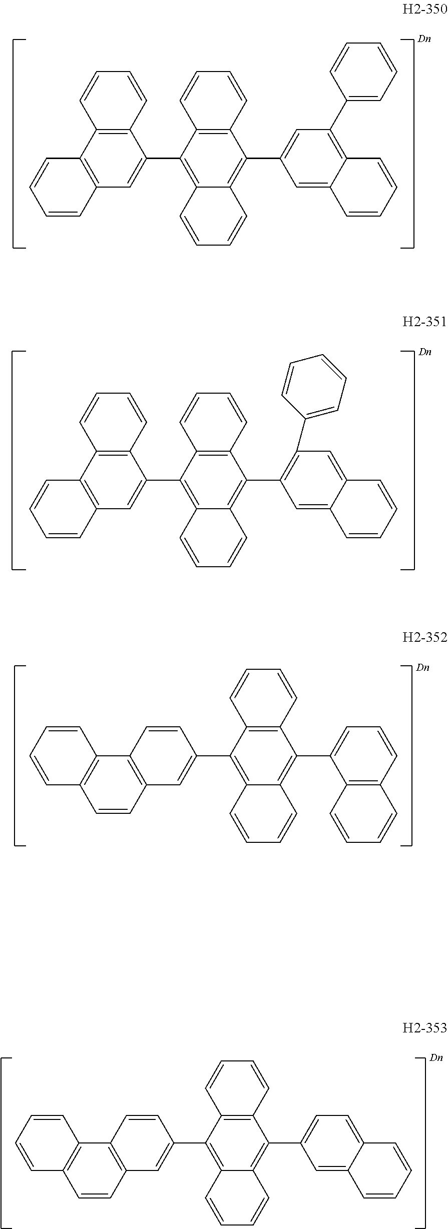 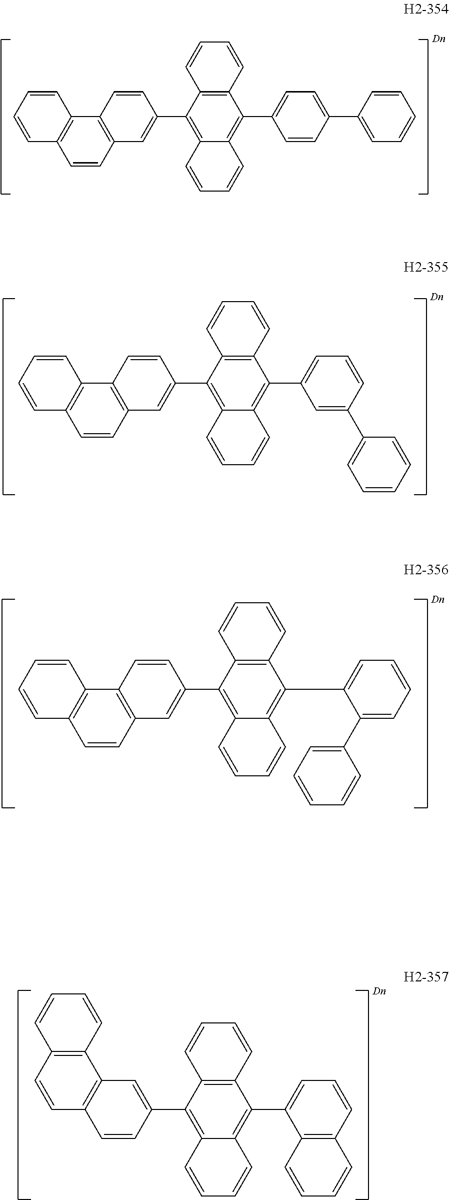 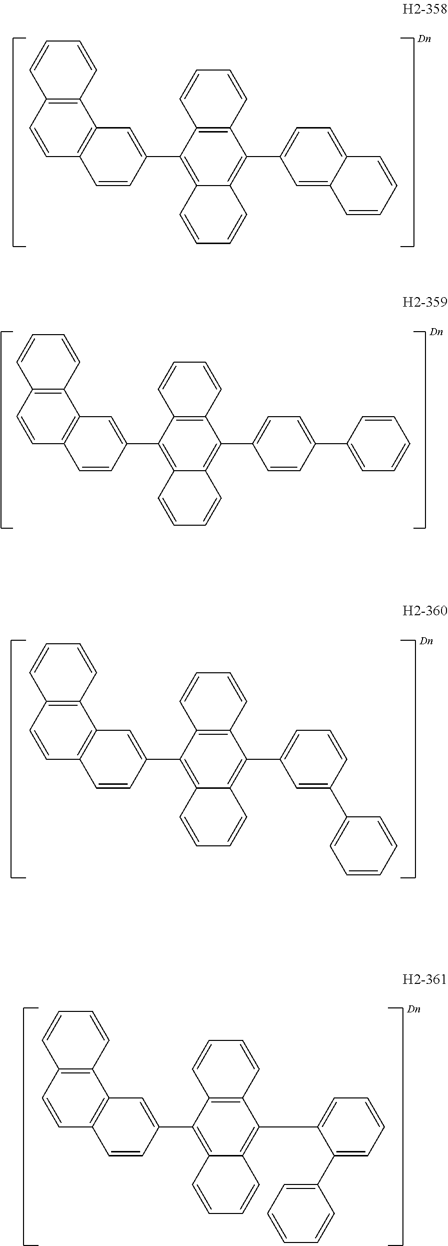 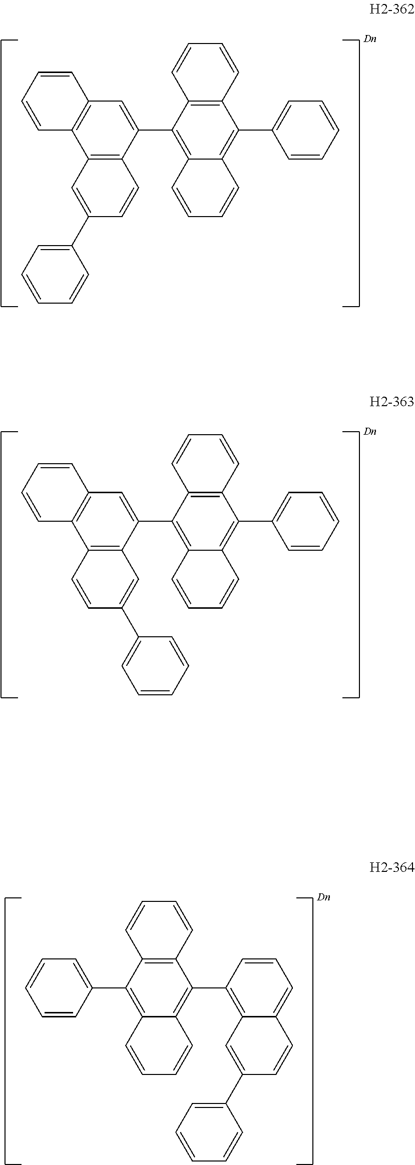 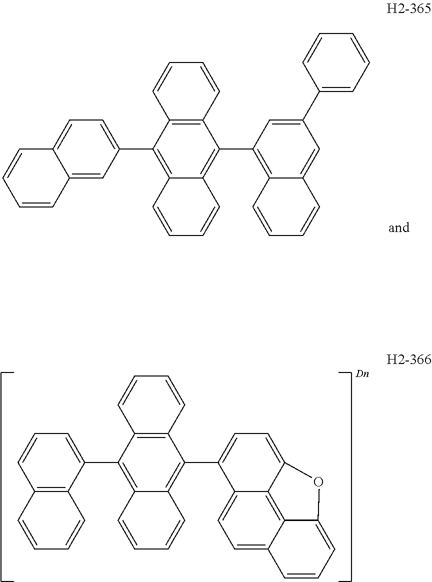
wherein “Dₙ” in the above compounds means that n number of hydrogens is replaced by deuterium, wherein n represents an integer of 1 or more and the upper limit of n is determined by the number of hydrogens that can be substituted in each compound.

9. An organic electroluminescent device comprising a first electrode; a second electrode; and at least one light-emitting layer between the first electrode and the second electrode, wherein the at least one light-emitting layer comprises a plurality of host materials according to claim 1.

10. An organic electroluminescent compound represented by the following Formula 11: 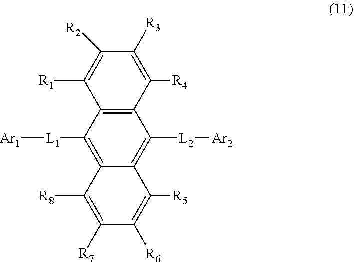
wherein,
R₁ to R₈ each independently represent hydrogen or deuterium; provided that at least one of R₁ to R₈ is deuterium,
L₁ and L₂ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl or a substituted or unsubstituted (3- to 30-membered)heteroaryl; and
Ar₂ is represented by the following Formula 1-A; 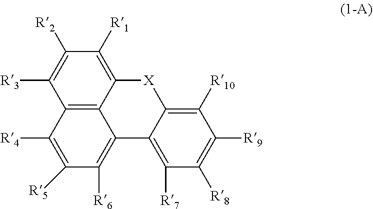
wherein,
X represents —NR″—, —Se—, —O—, or —S—;
R′₁ to R′₁₀ each independently represent hydrogen, deuterium, a substituted or unsubstituted (C₆-C₃₀)aryl, or a substituted or unsubstituted (3- to 30-membered)heteroaryl; or
may be linked to the adjacent substituents to form a substituted or unsubstituted (3- to 30-membered) mono- or polycyclic, alicyclic, aromatic ring, or a combination thereof; provided that at least one of R′₁ to R′₁₀ is deuterium; and
any one of R′₁ to R′₁₀ is linked to L₂;
provided that the cases where all hydrogens in the compound are replaced with deuterium are excluded.

11. The organic electroluminescent compound according to claim 10, wherein at least one of R₁, R₄, R₅, and R₈ is hydrogen.

12. The organic electroluminescent compound according to claim 10, wherein the residual rate of hydrogen in the compound is 20% to 60% of the total amount of hydrogen.

13. The organic electroluminescent compound according to claim 10, wherein the residual rate of hydrogen in the compound is 40% to 60% of the total amount of hydrogen.

14. The organic electroluminescent compound according to claim 10, wherein the compound represented by Formula 11 is selected from the following compounds: 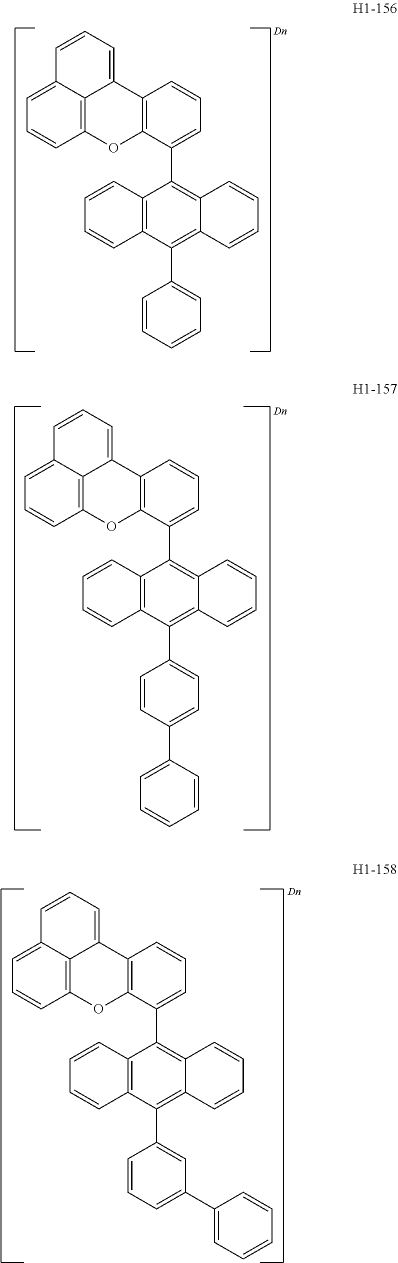 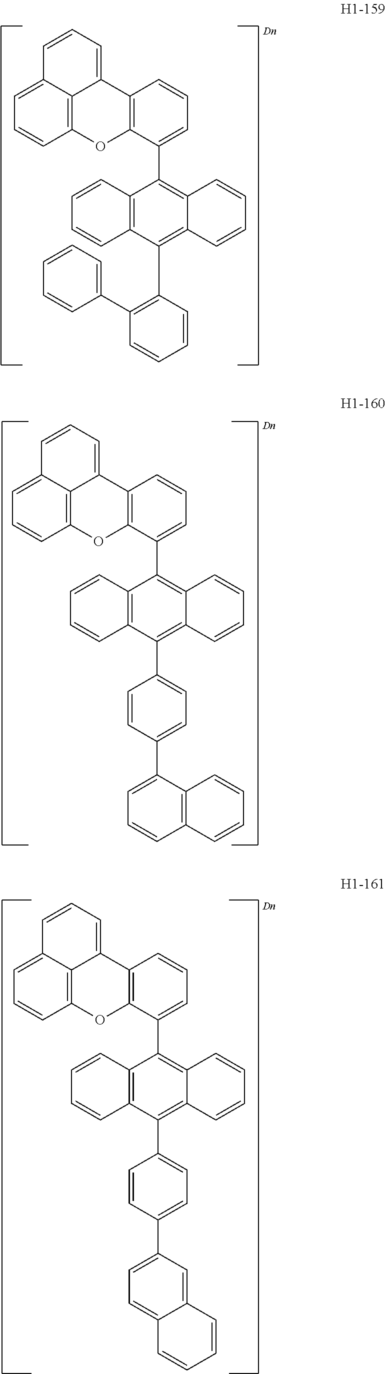 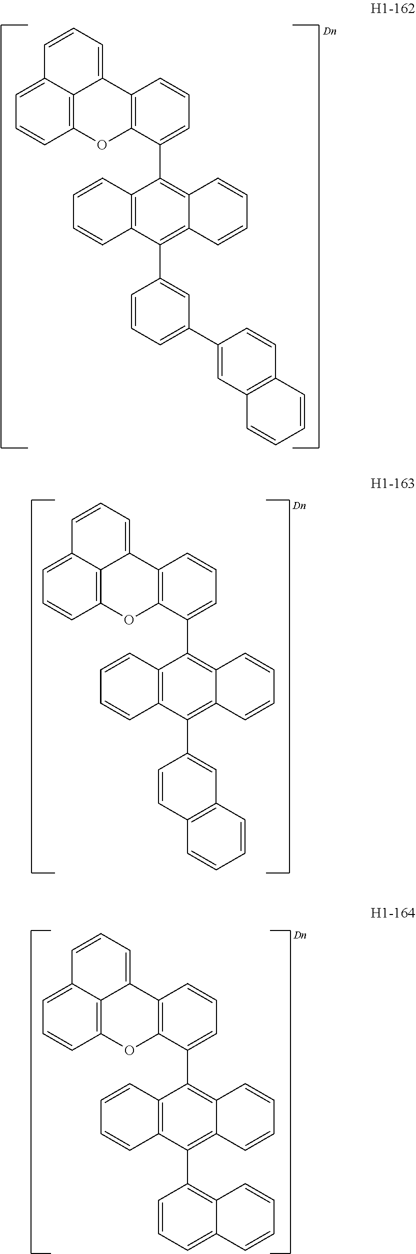 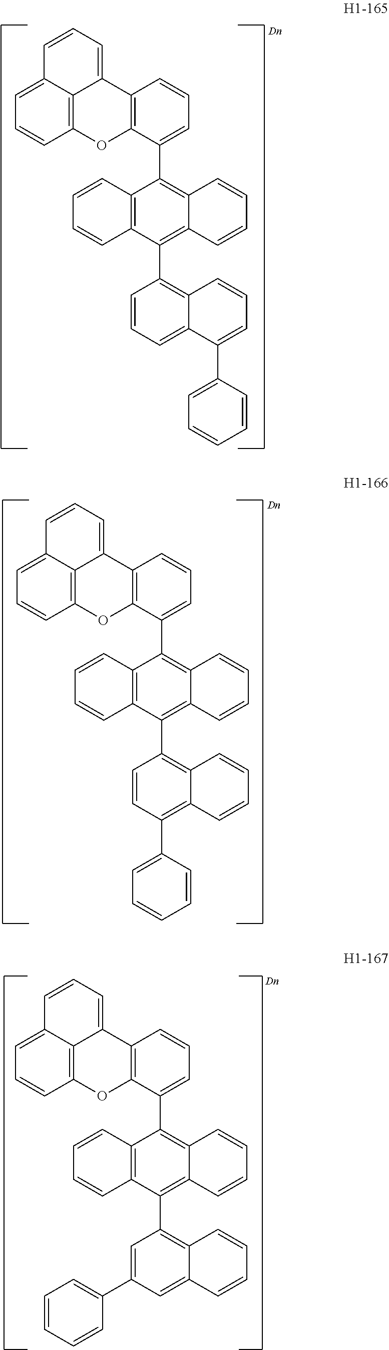 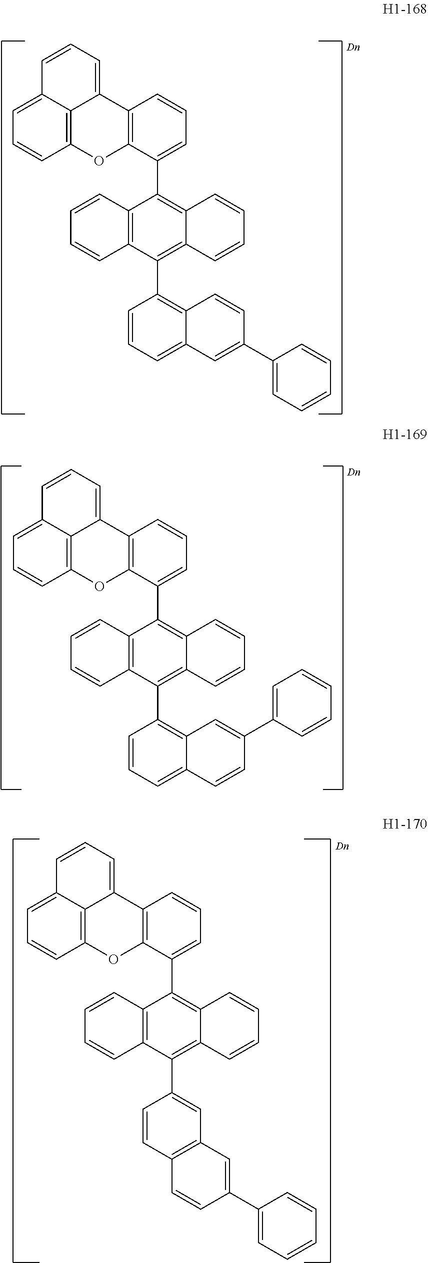 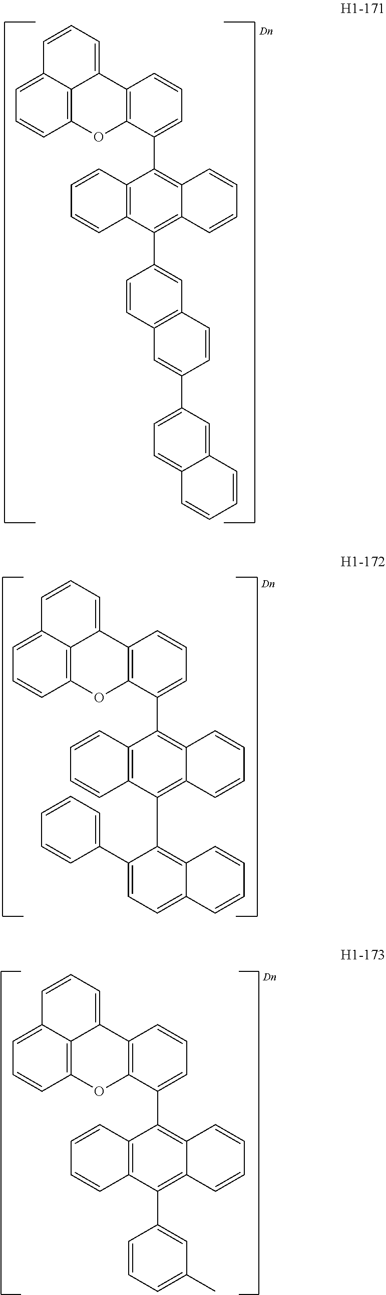 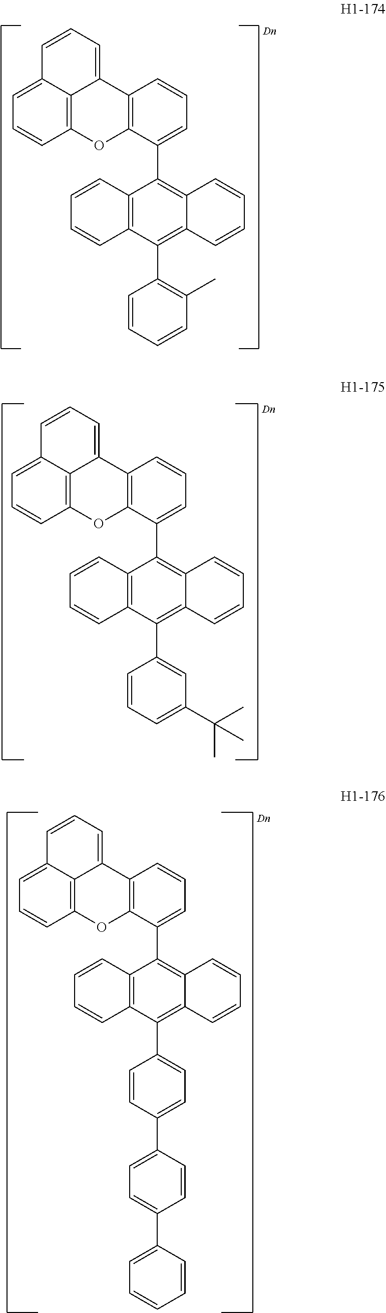 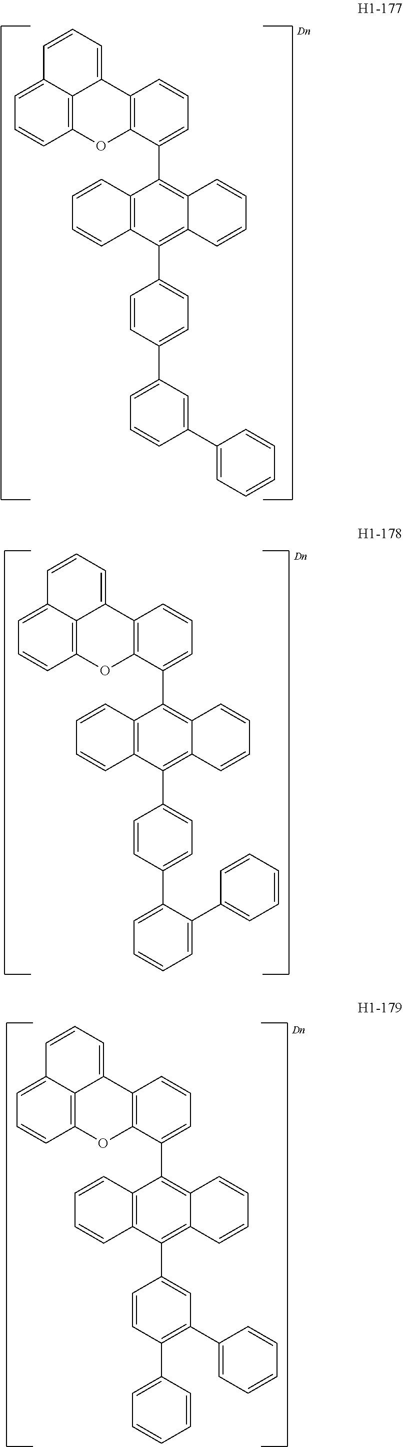 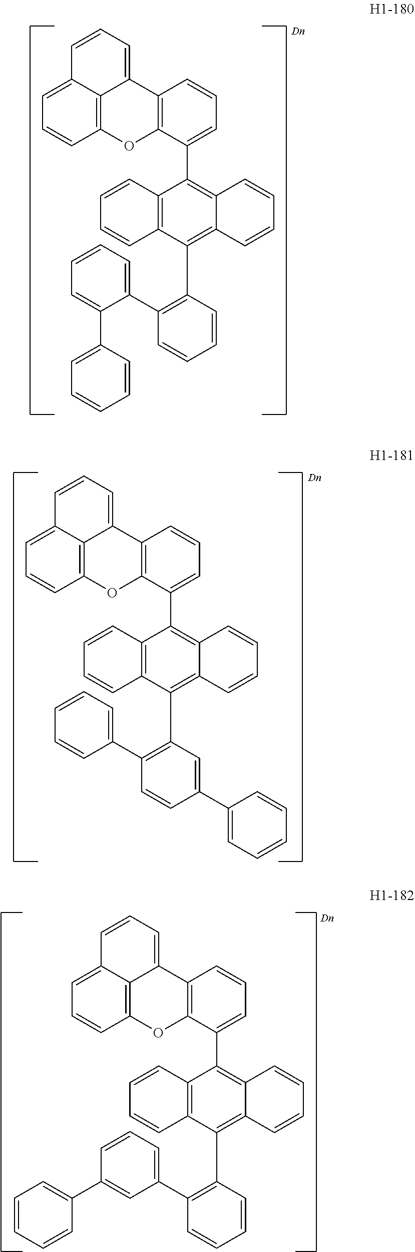 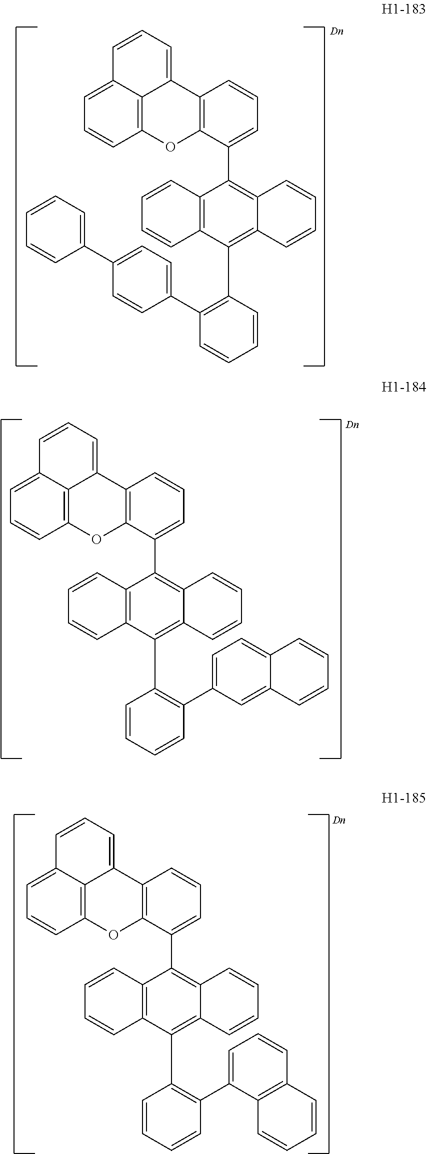 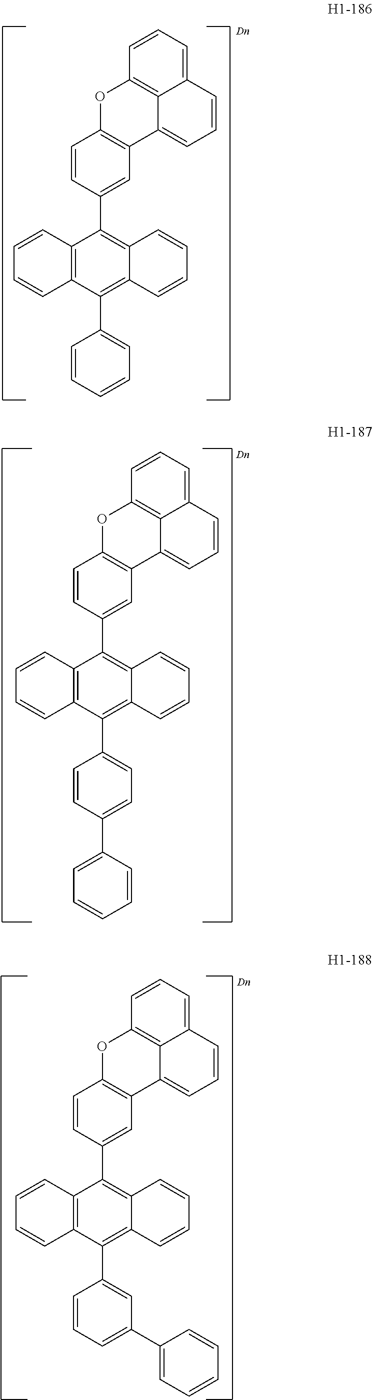 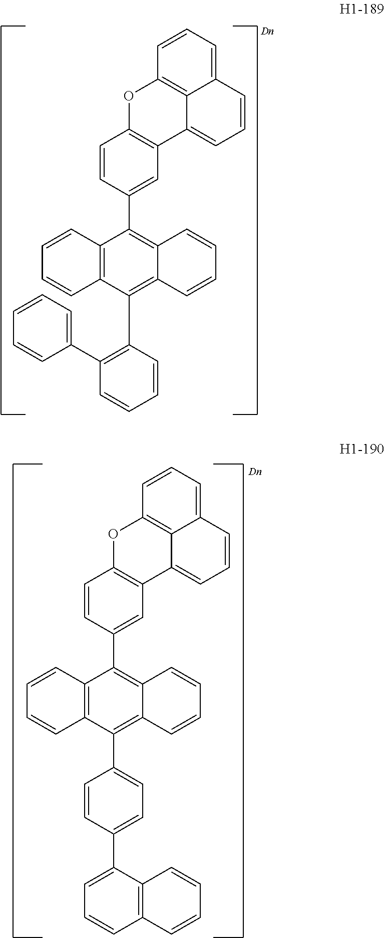 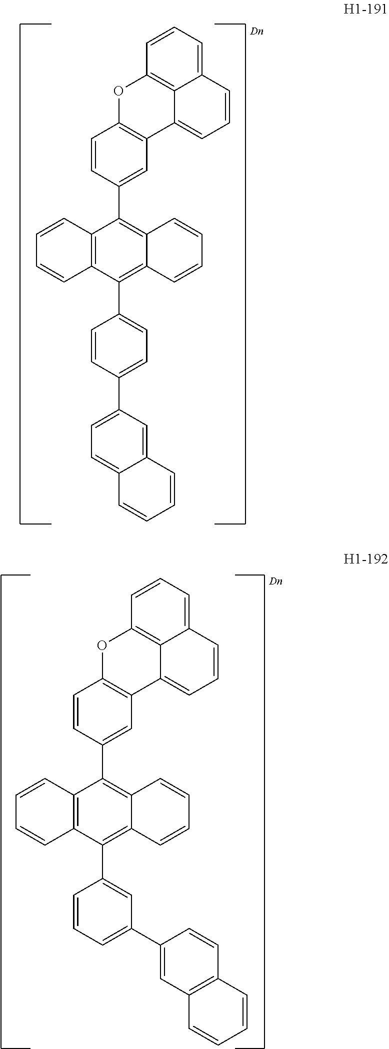 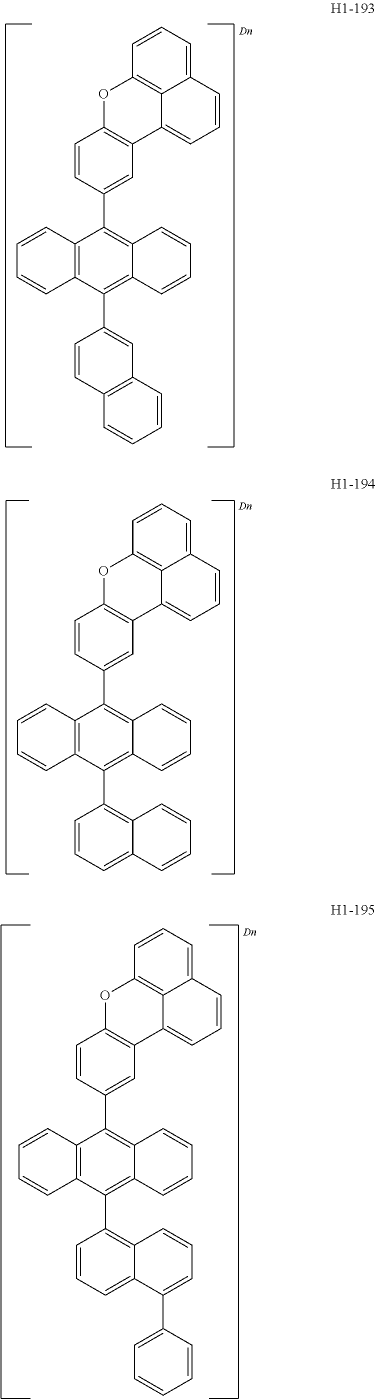 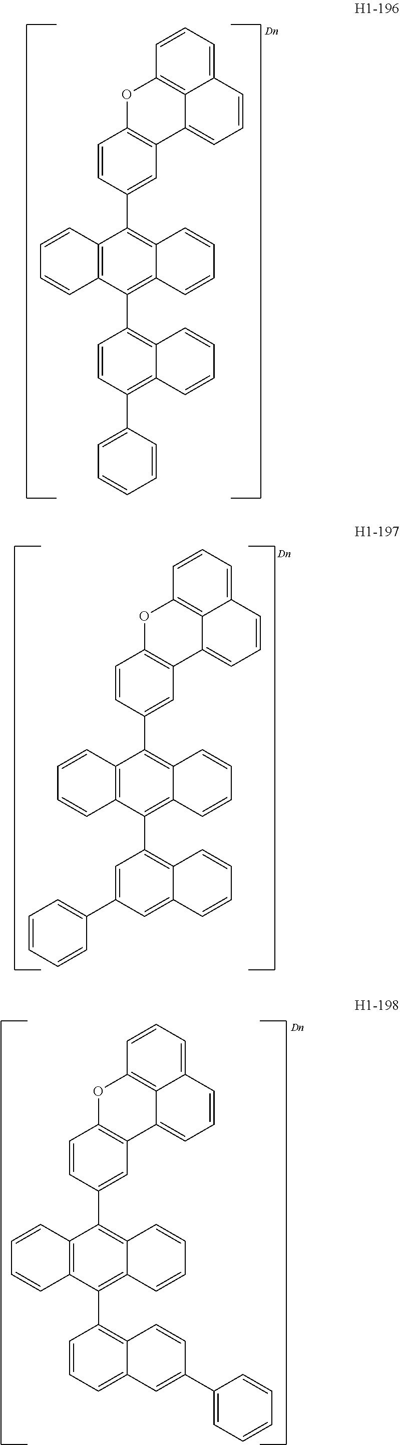 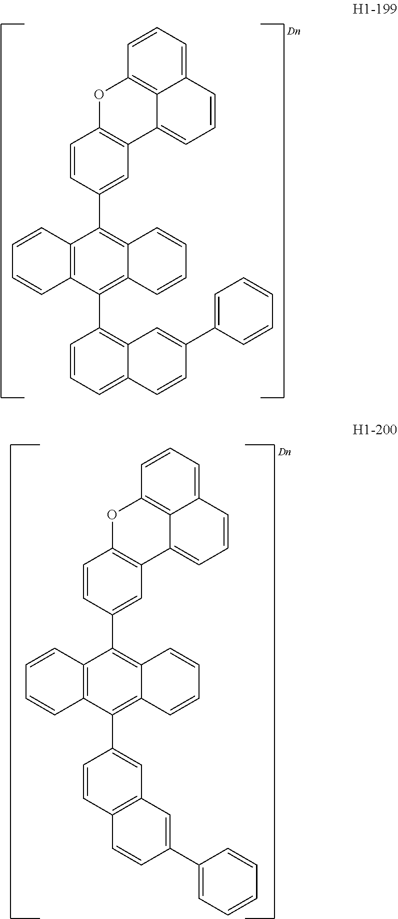 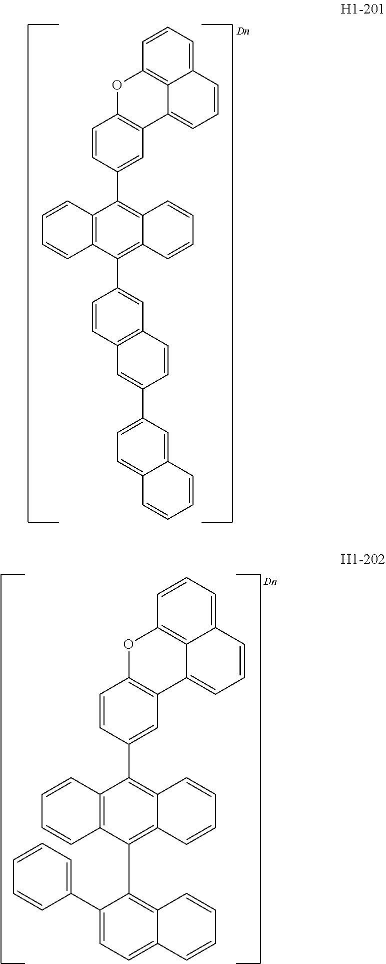 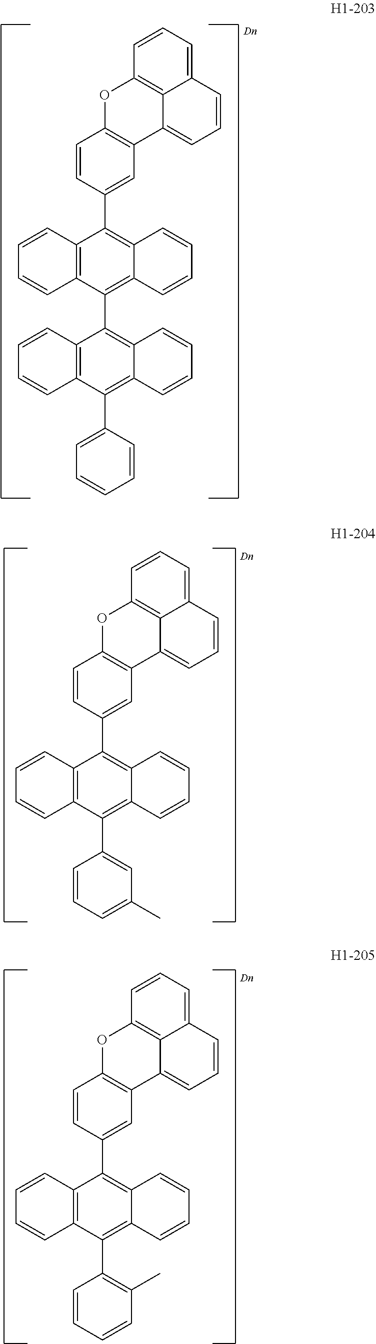 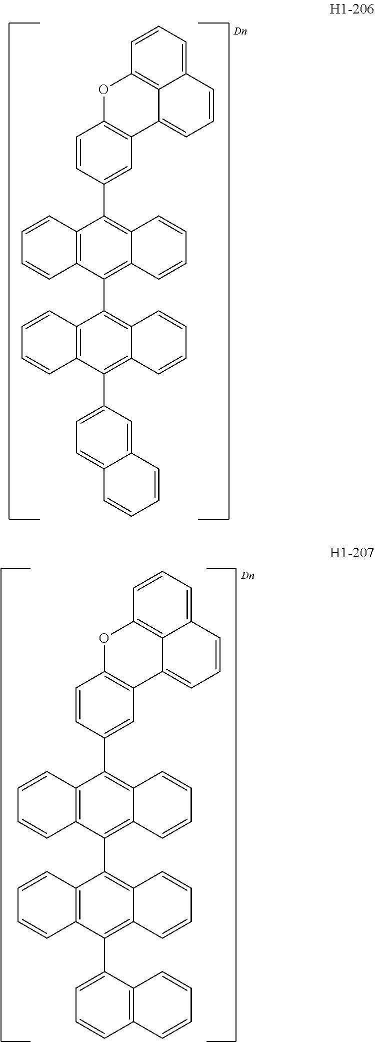 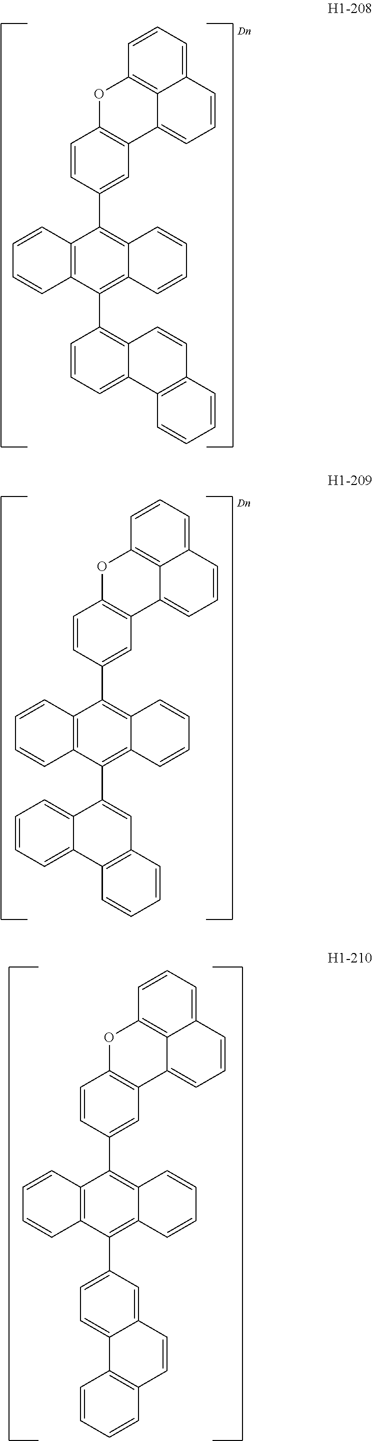 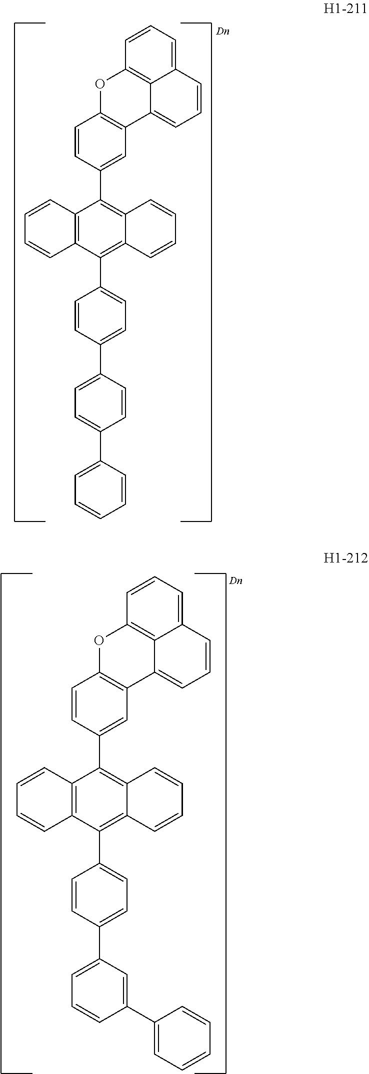 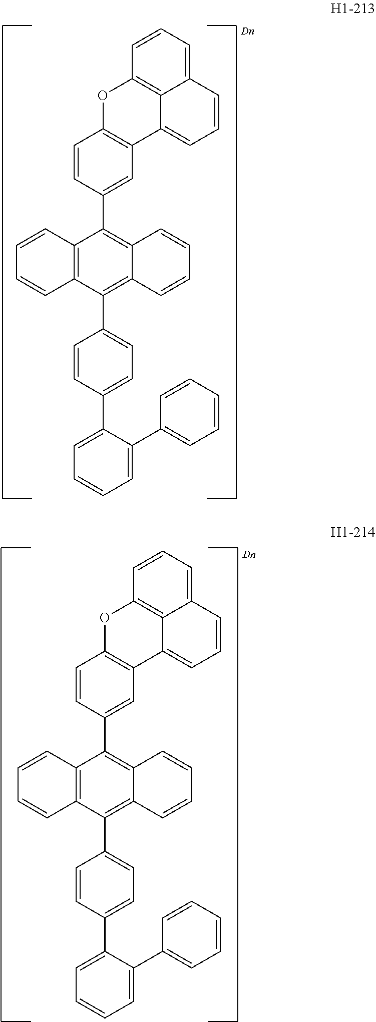 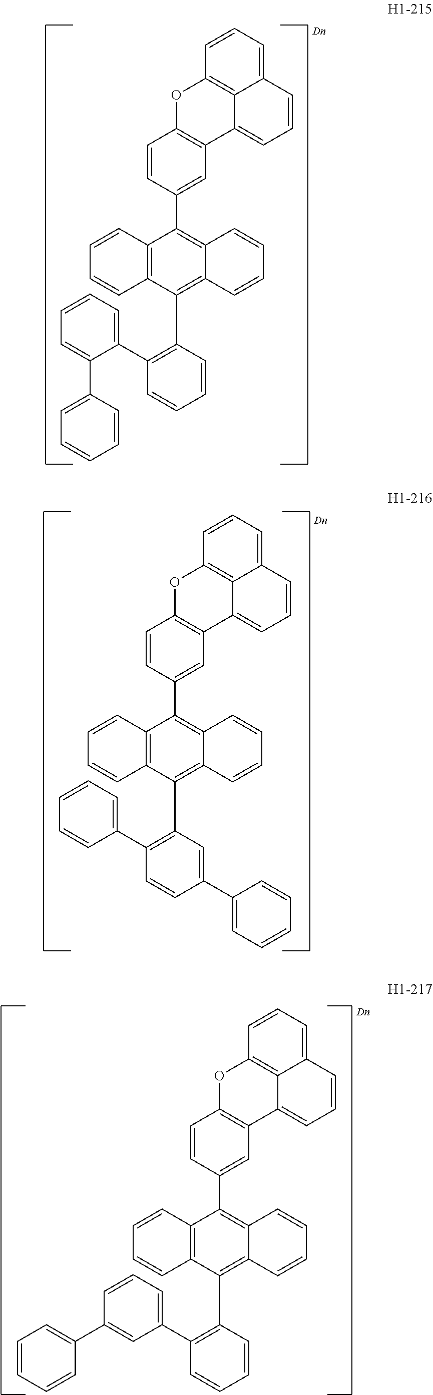 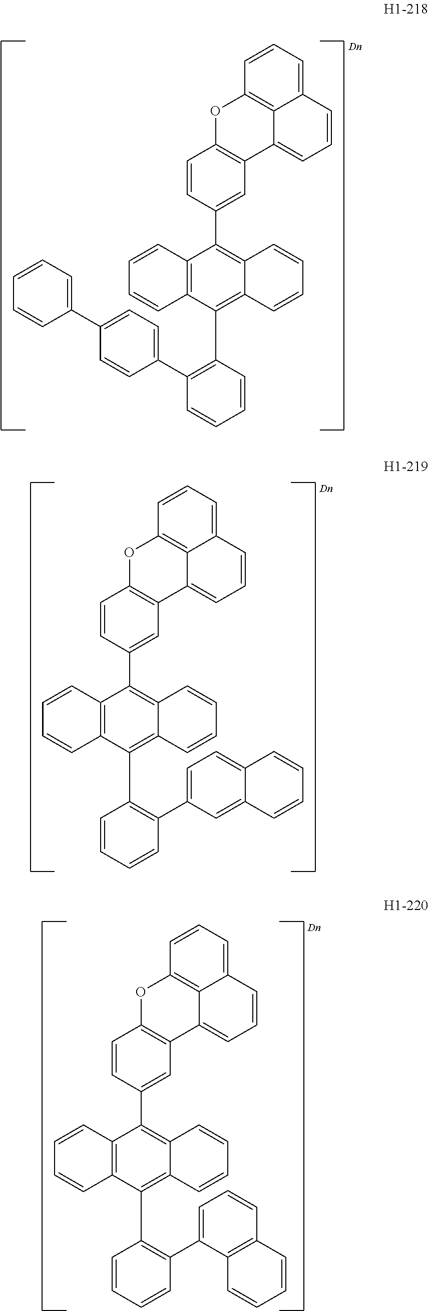 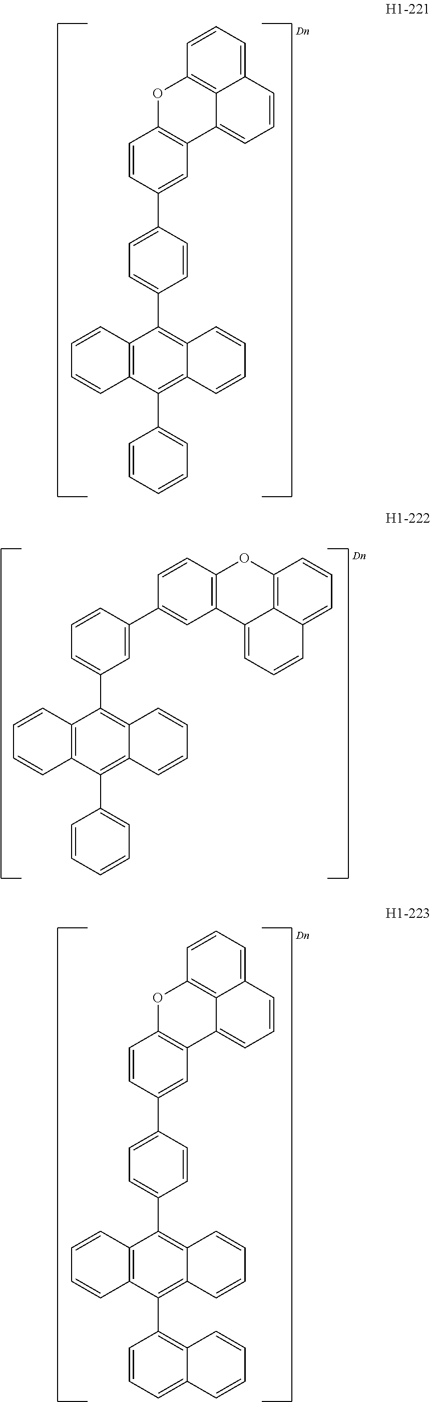 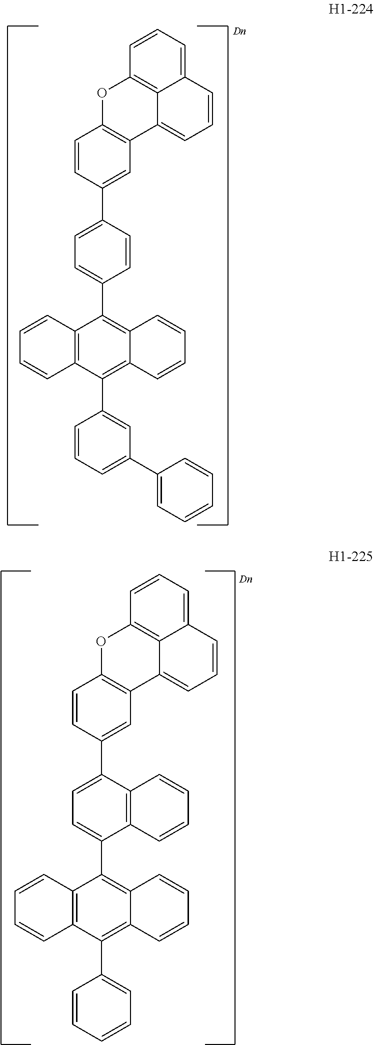 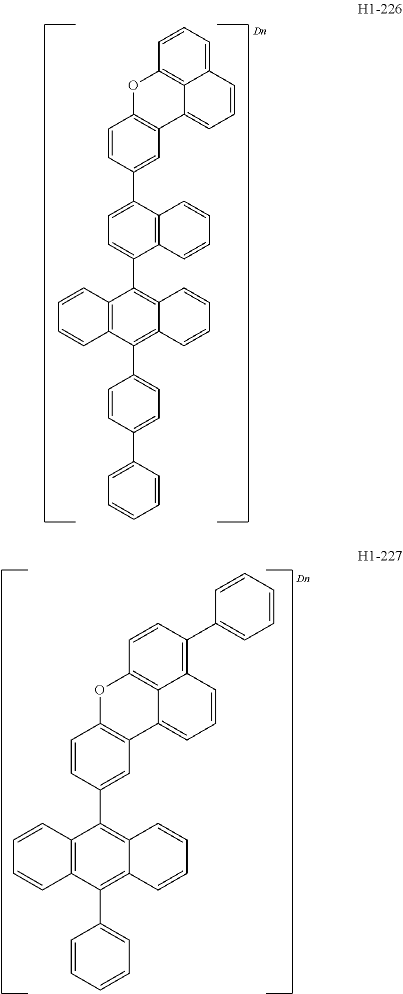 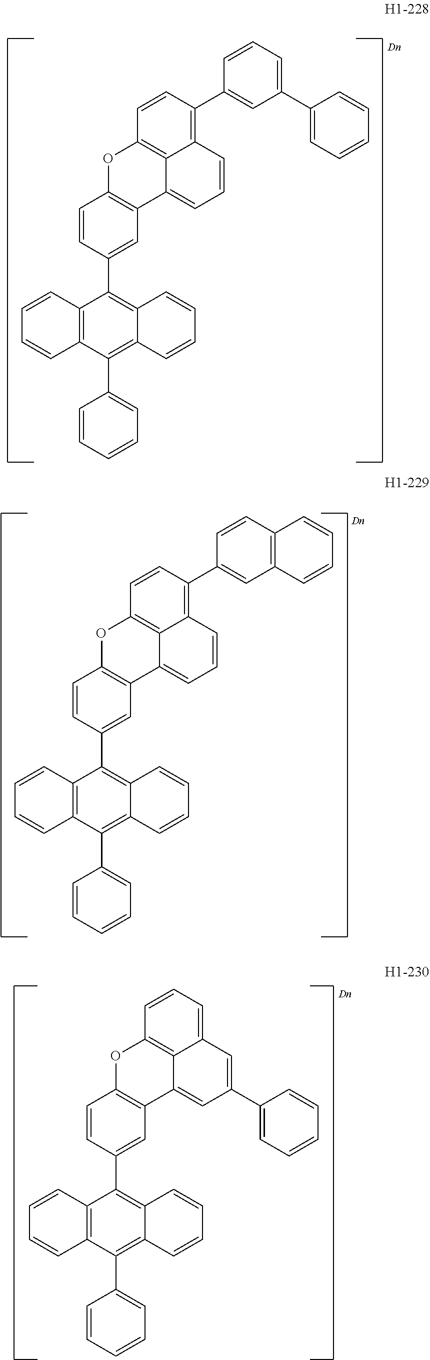 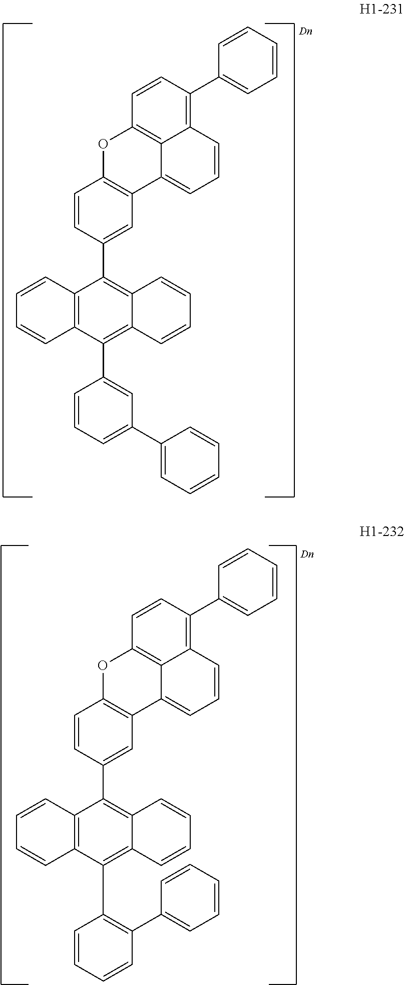 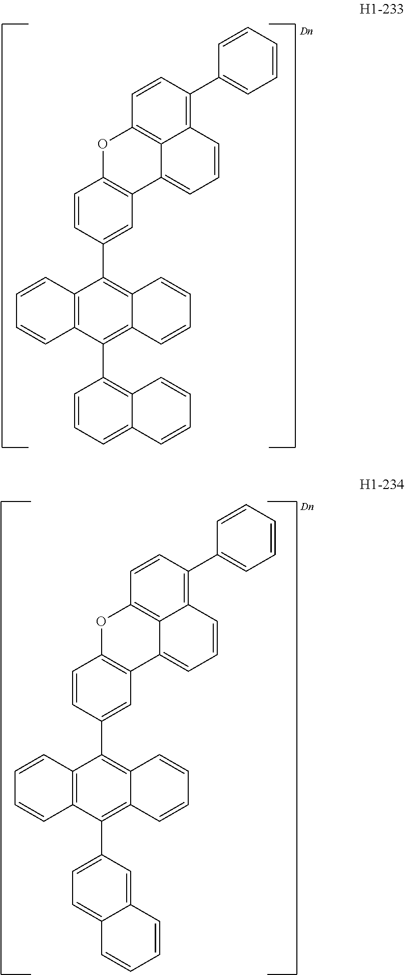 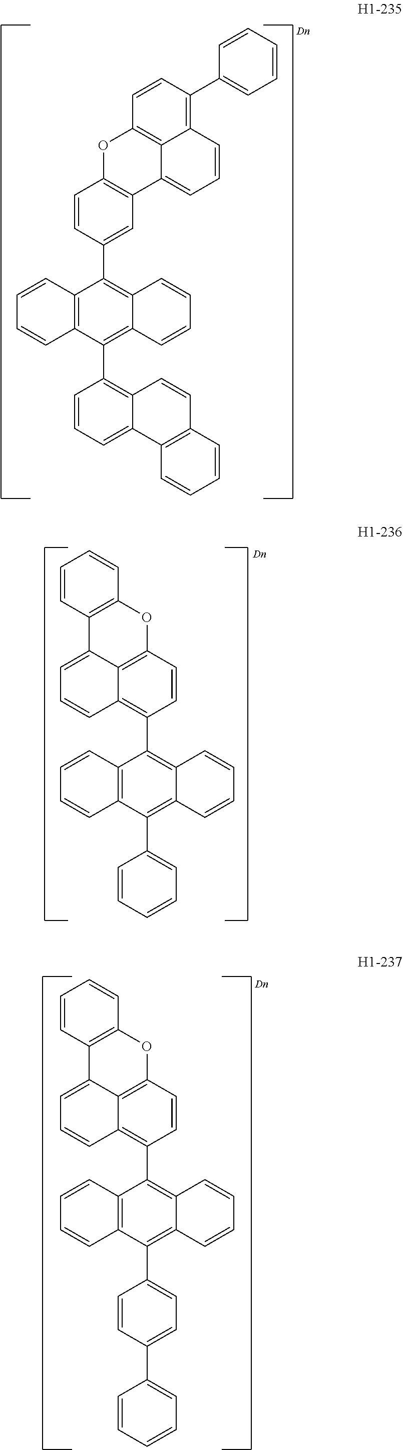 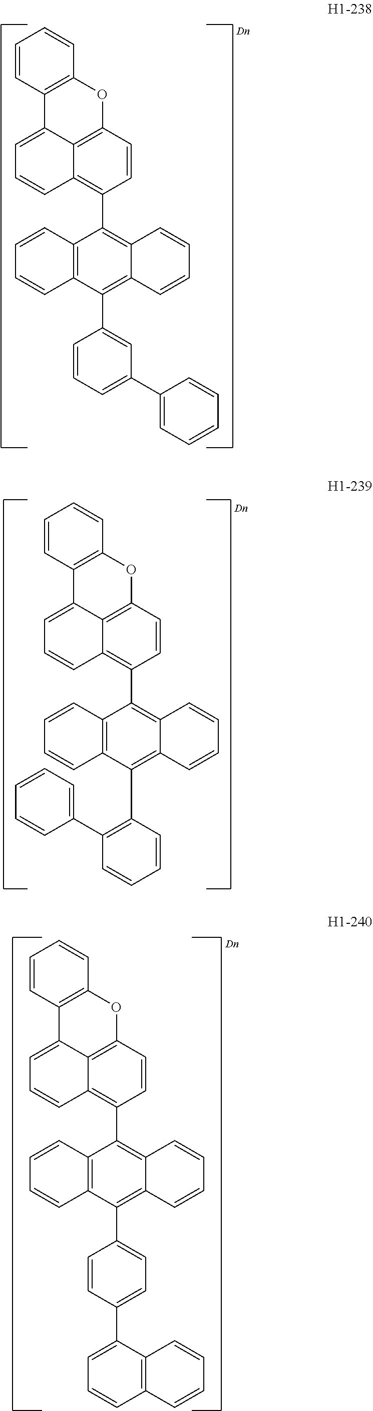 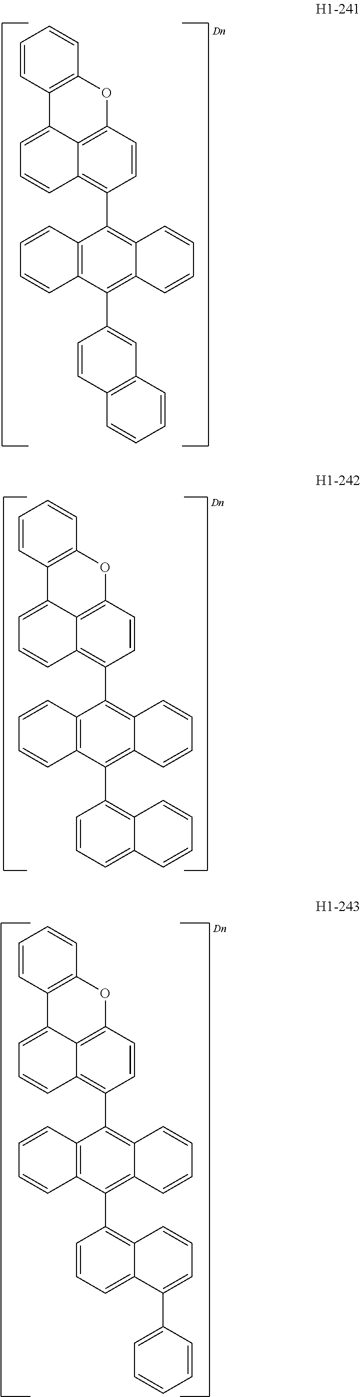 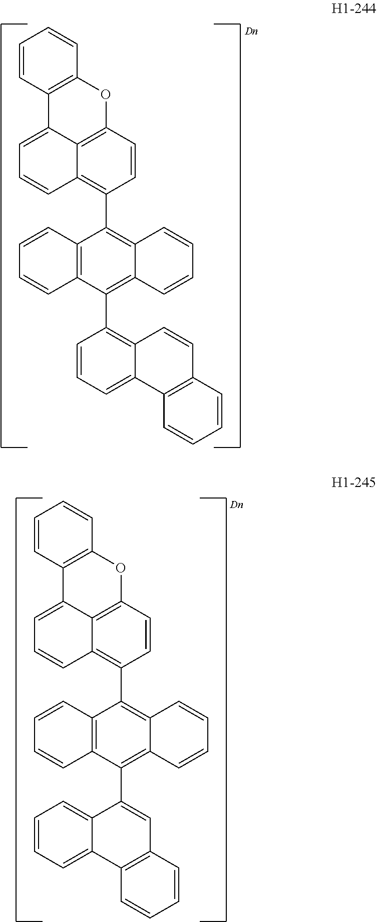 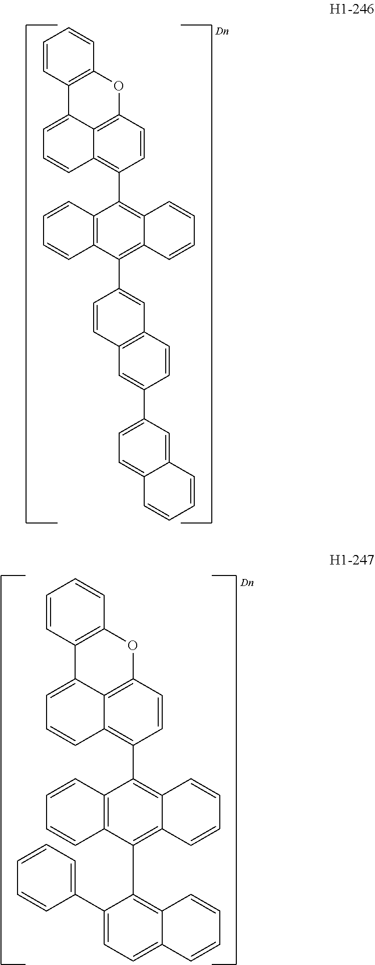 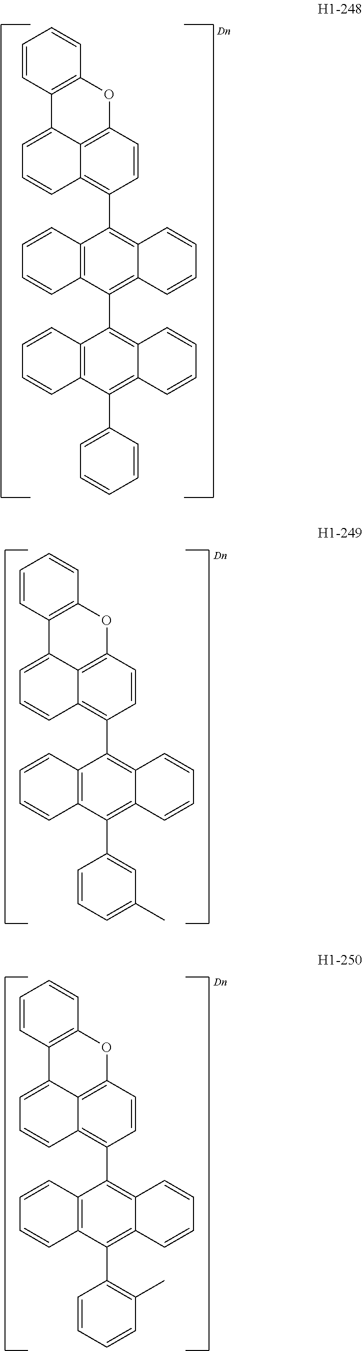 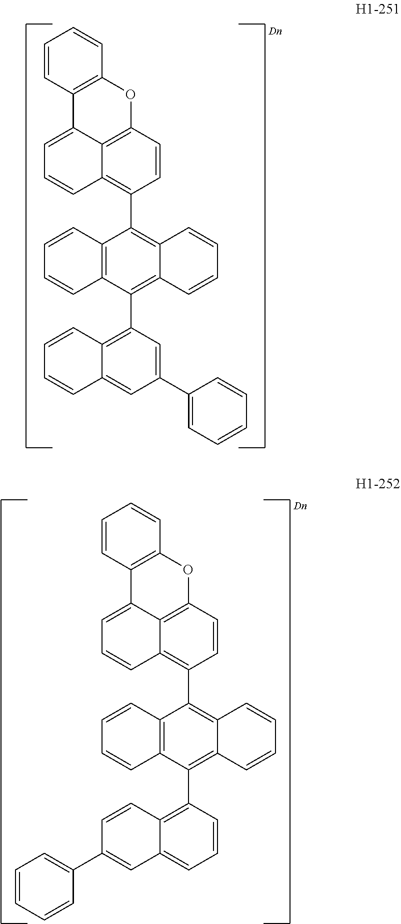 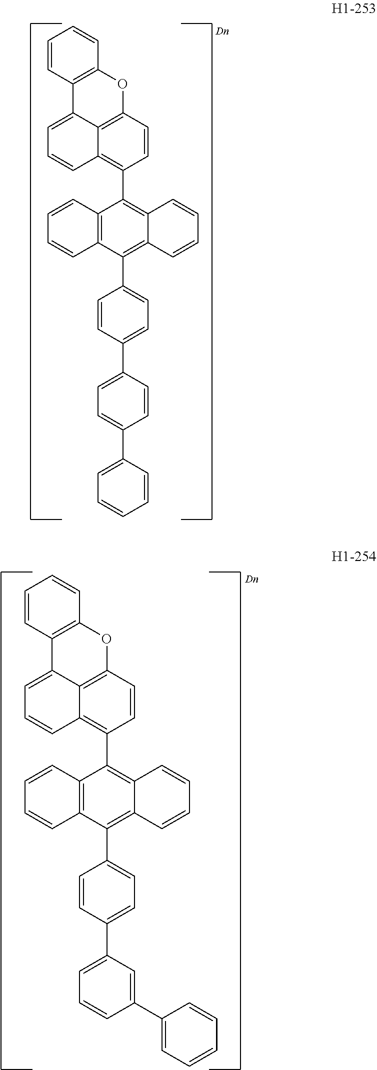 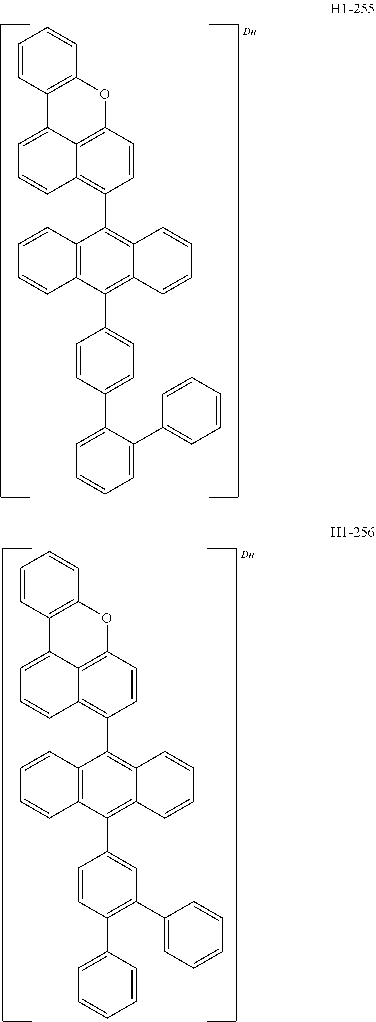 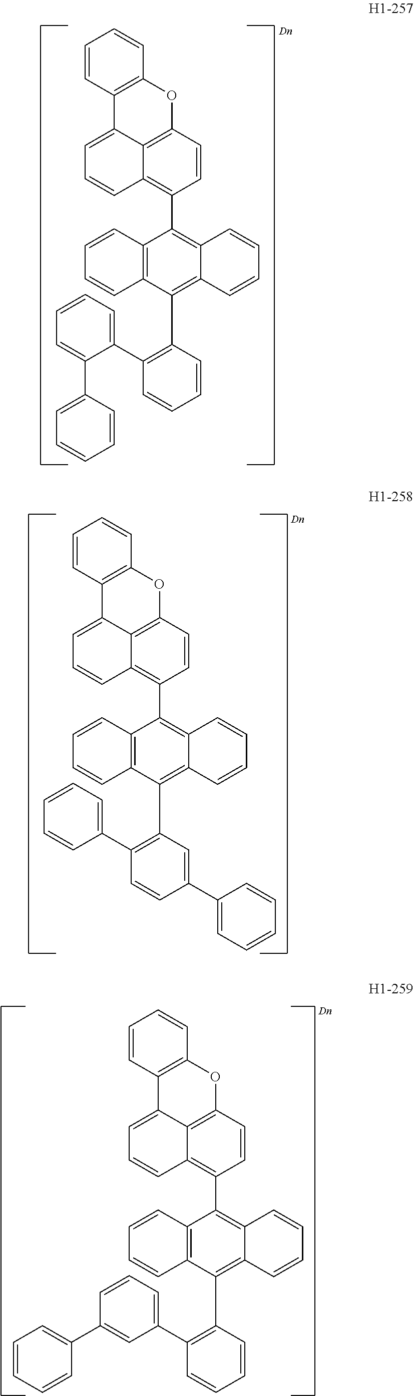 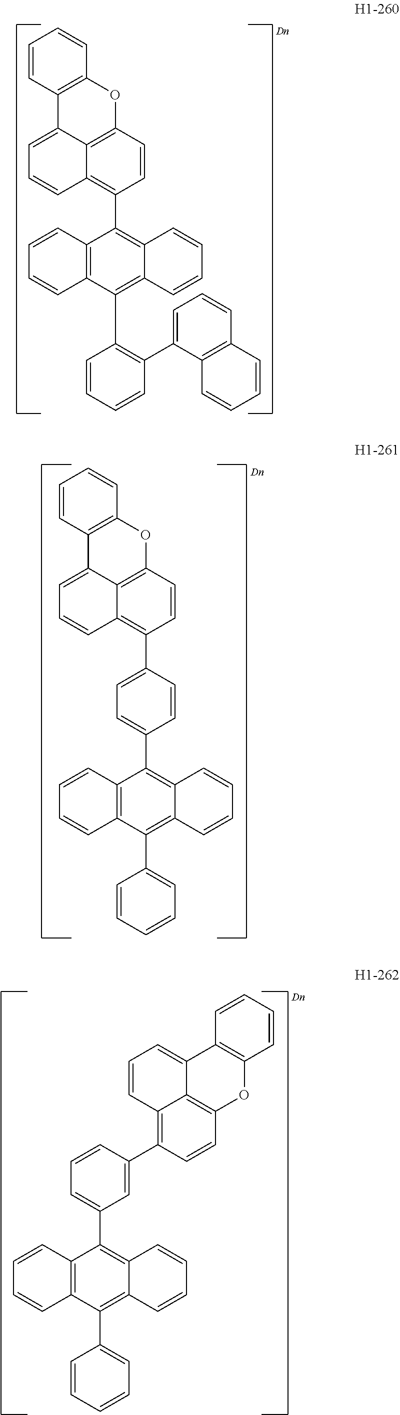 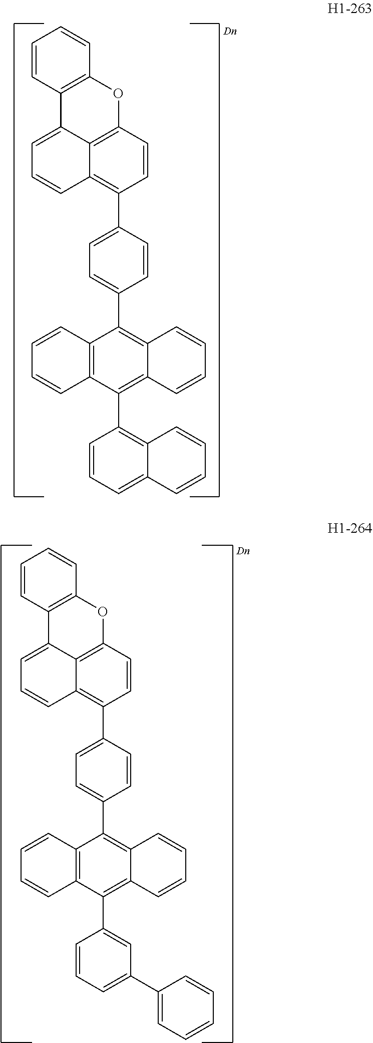 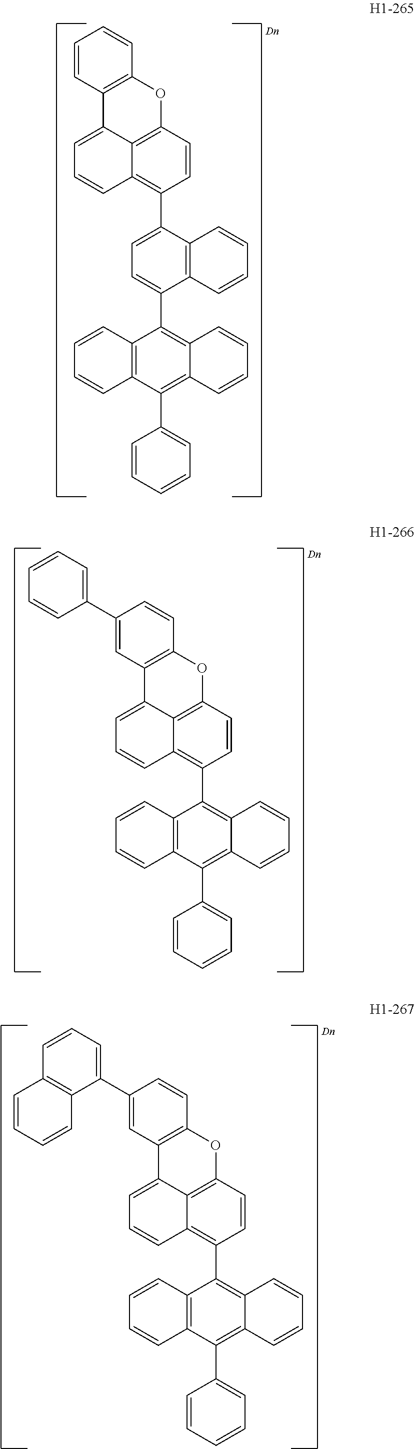 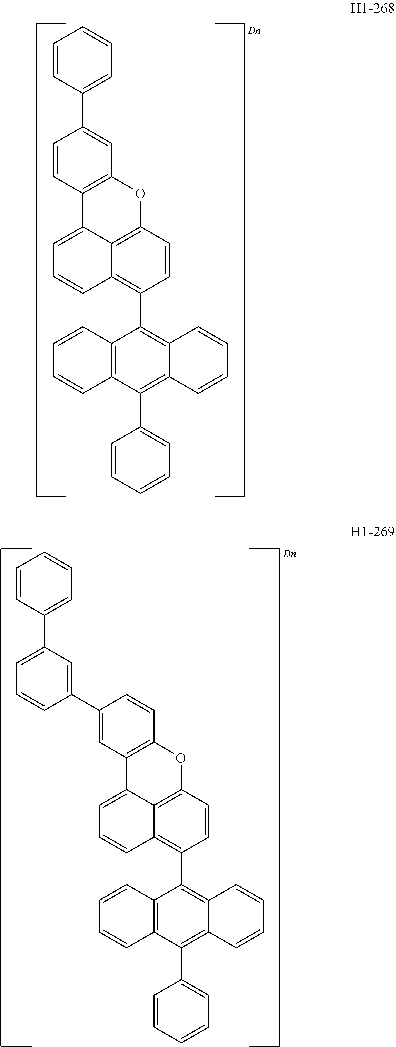 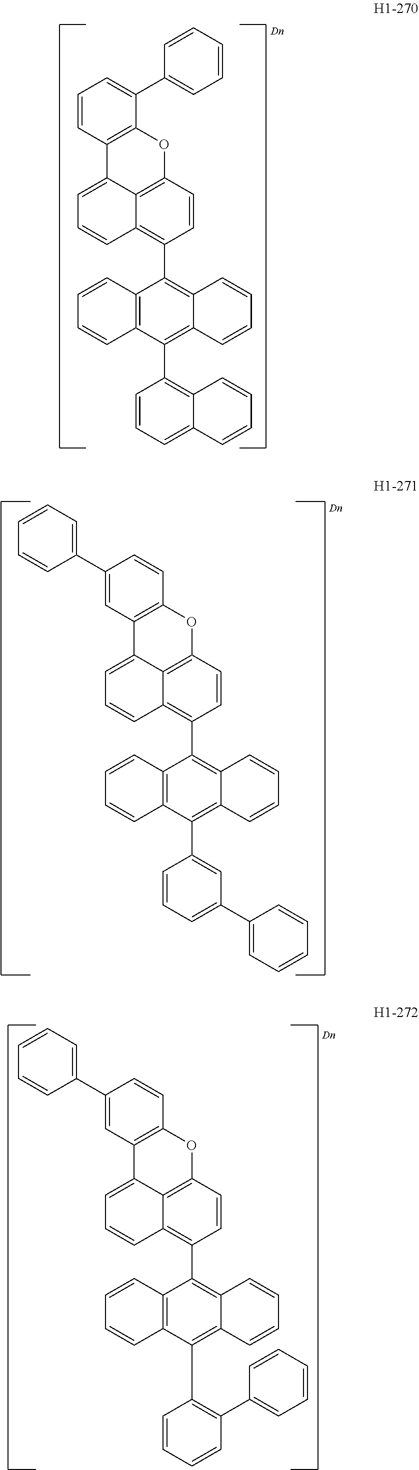 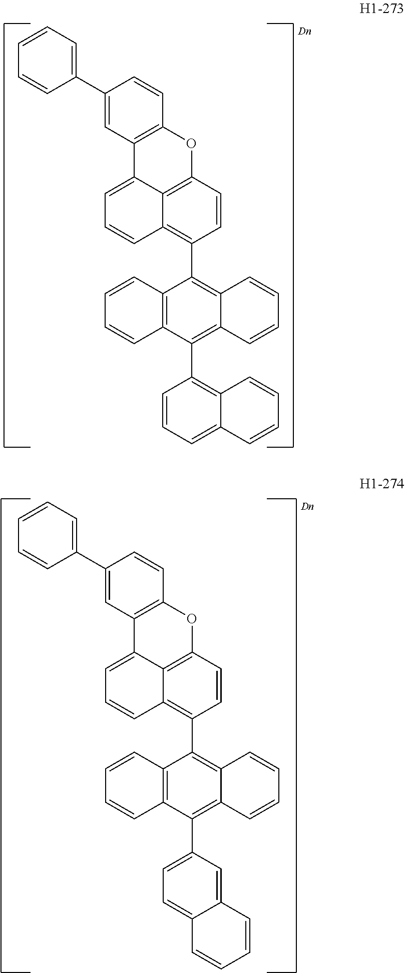 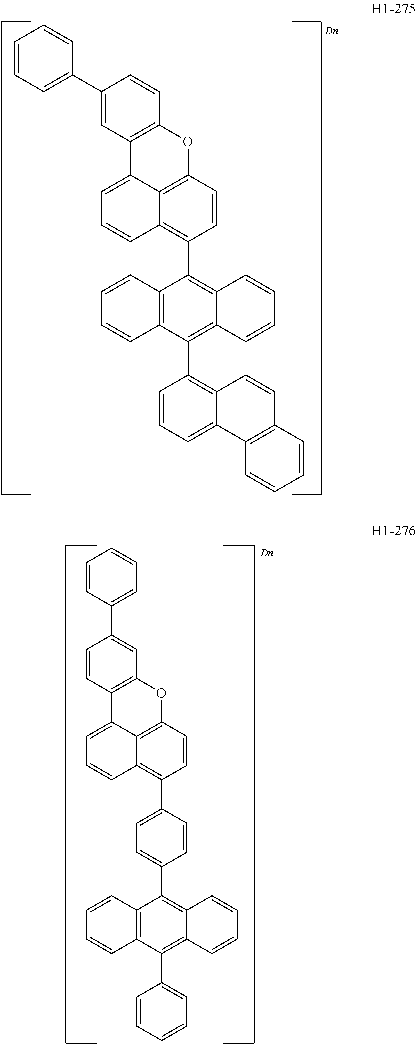 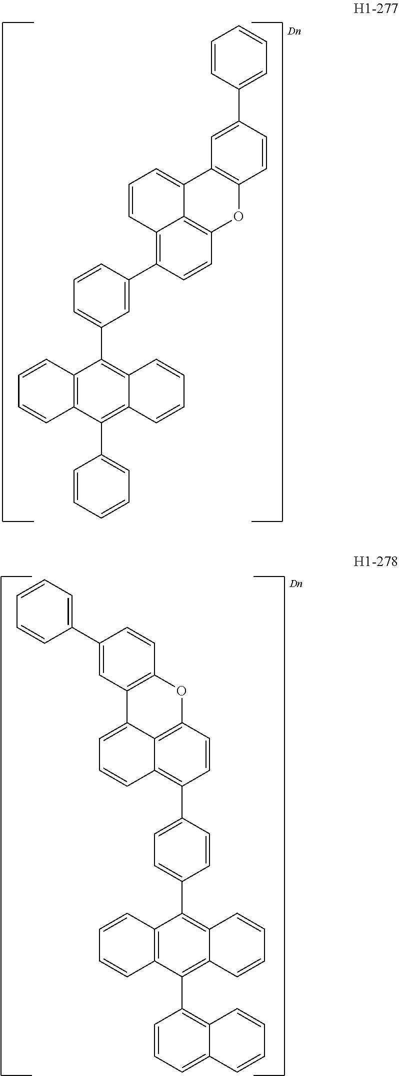 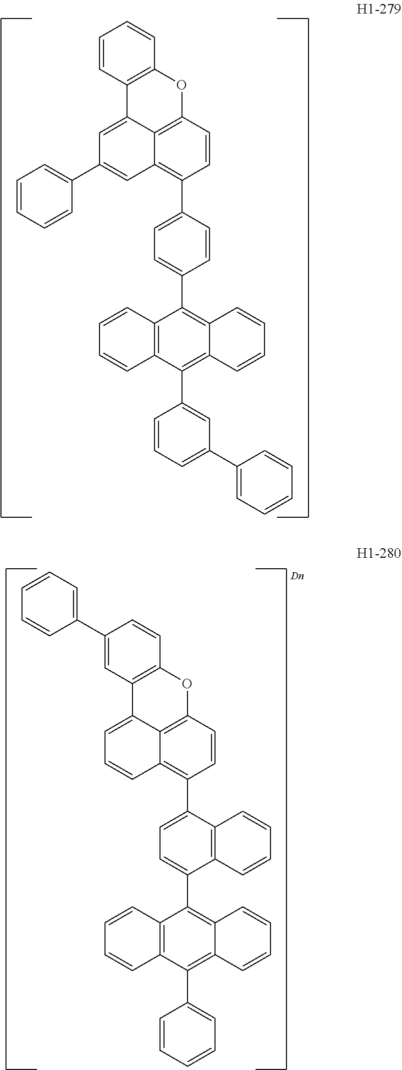 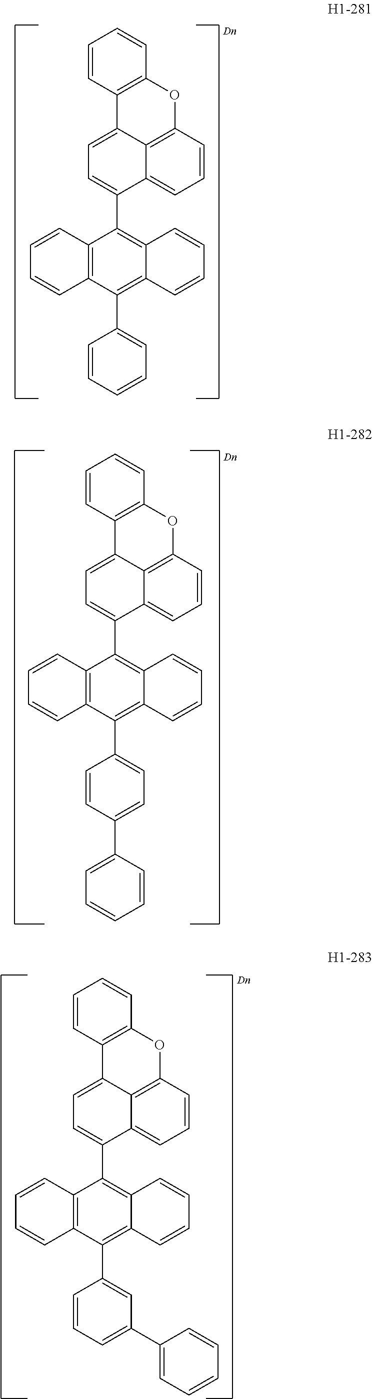 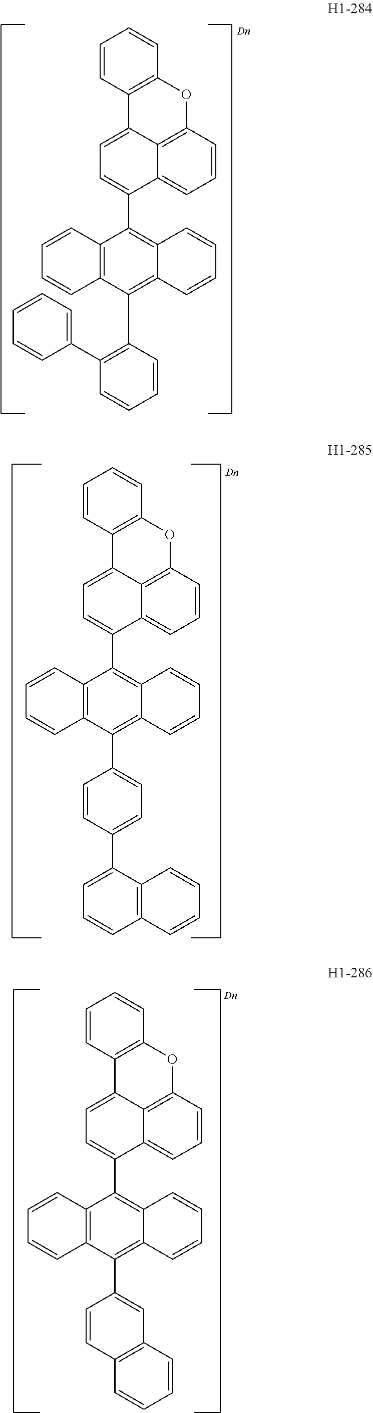 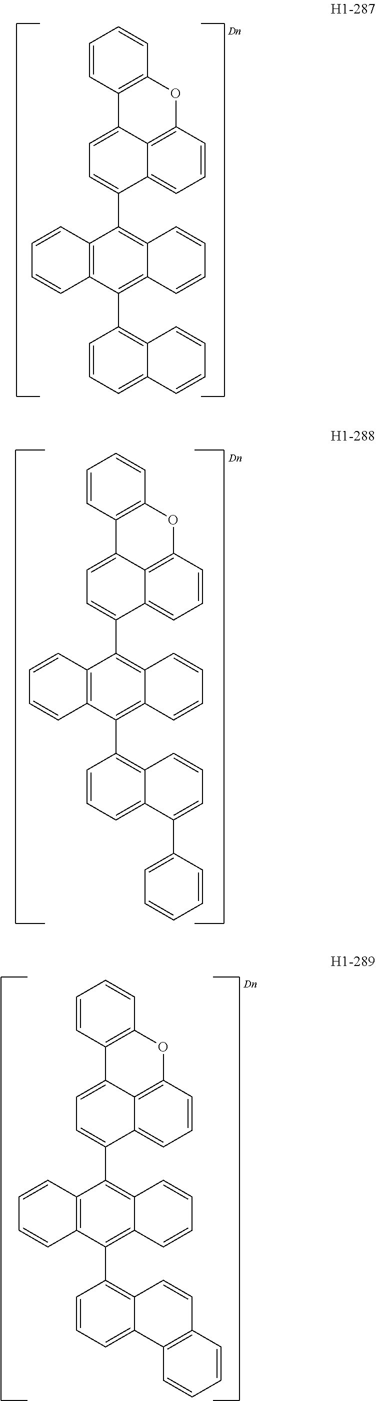 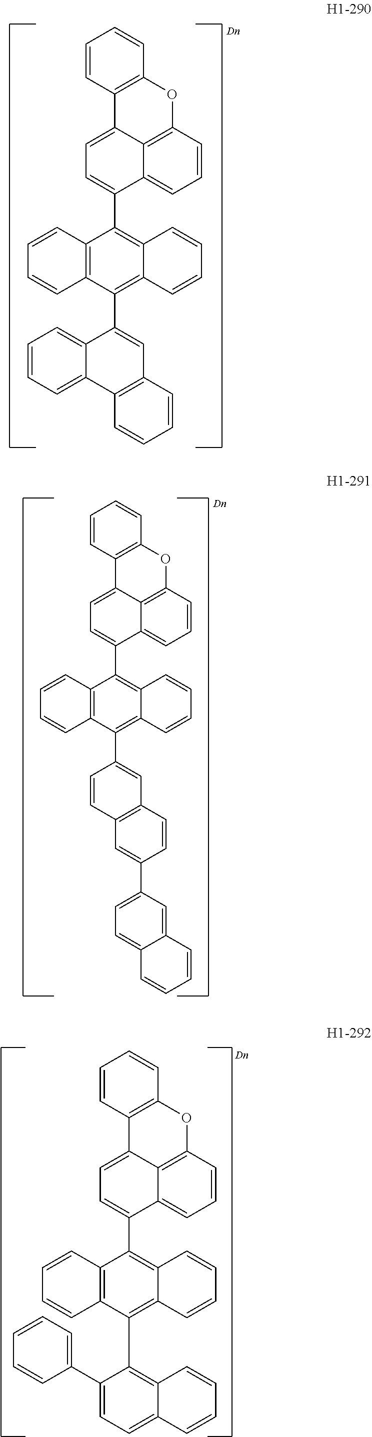 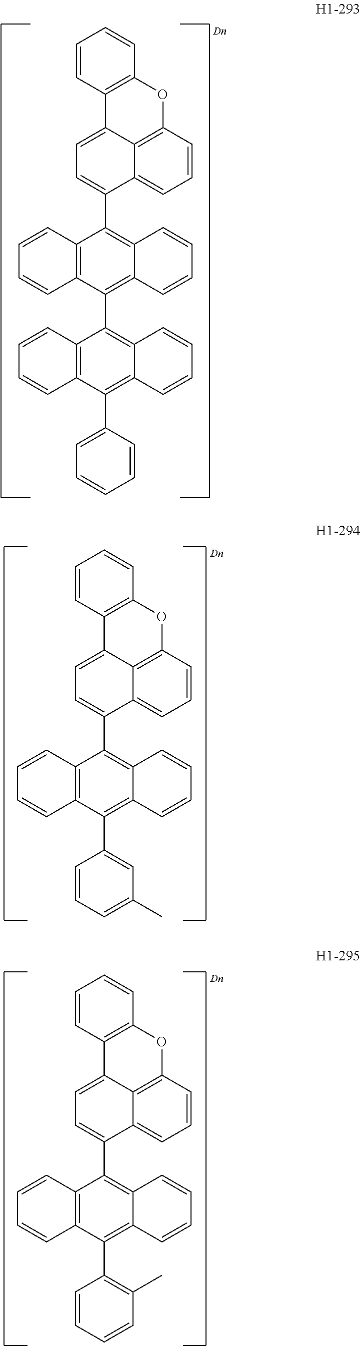 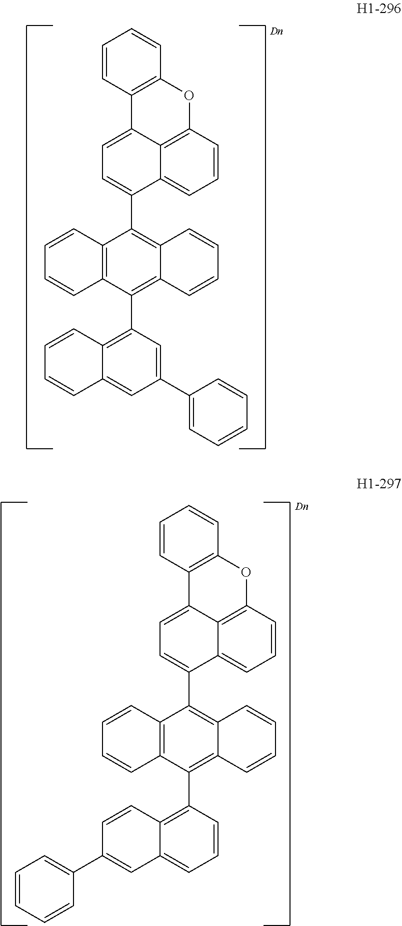 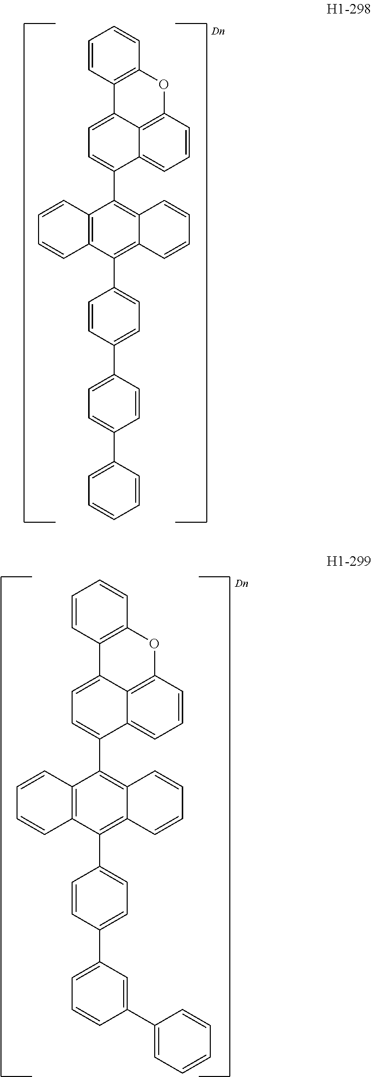 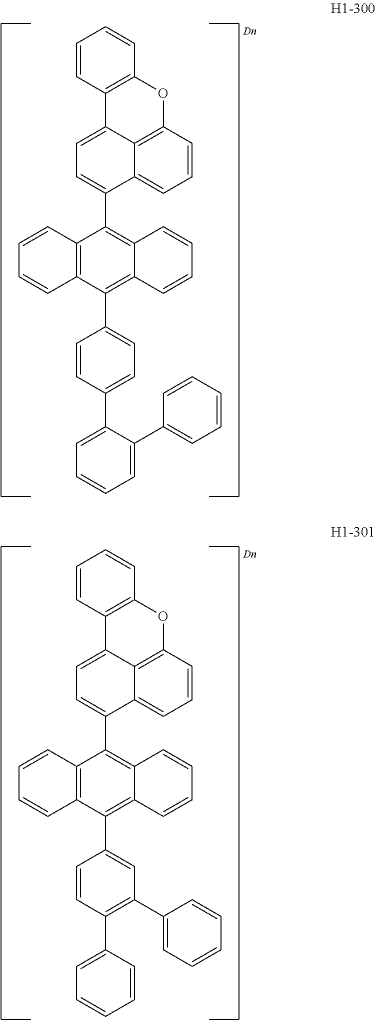 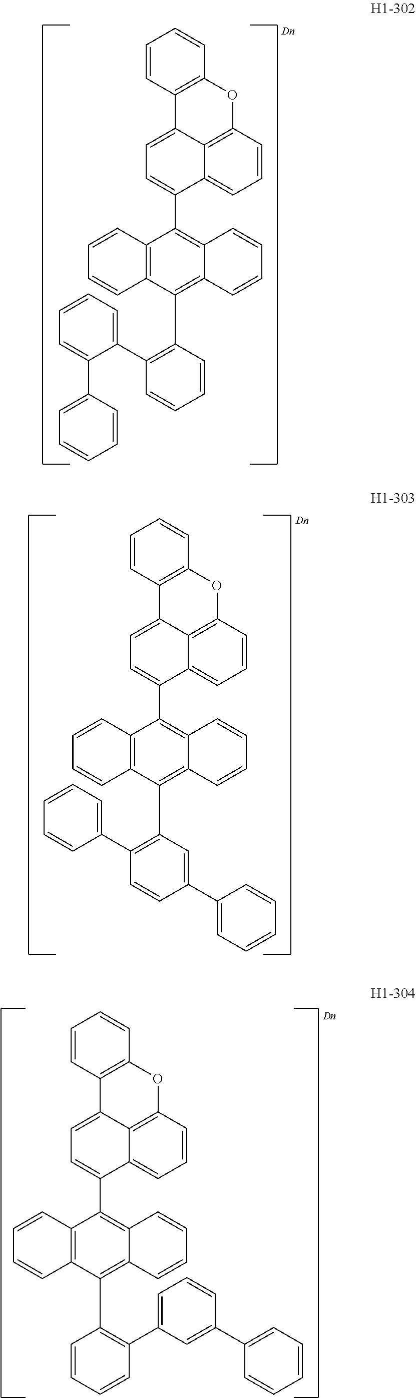 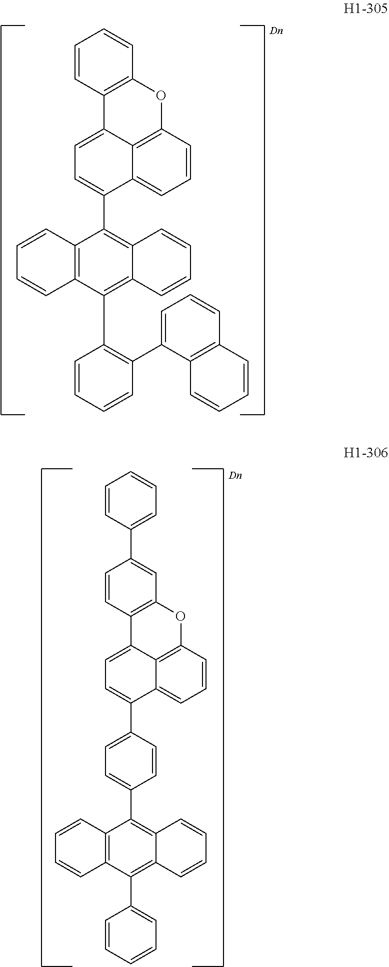 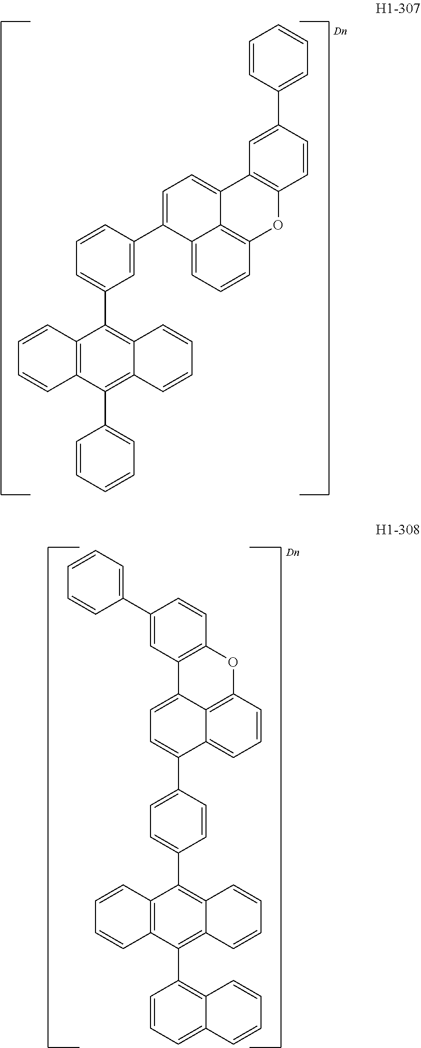 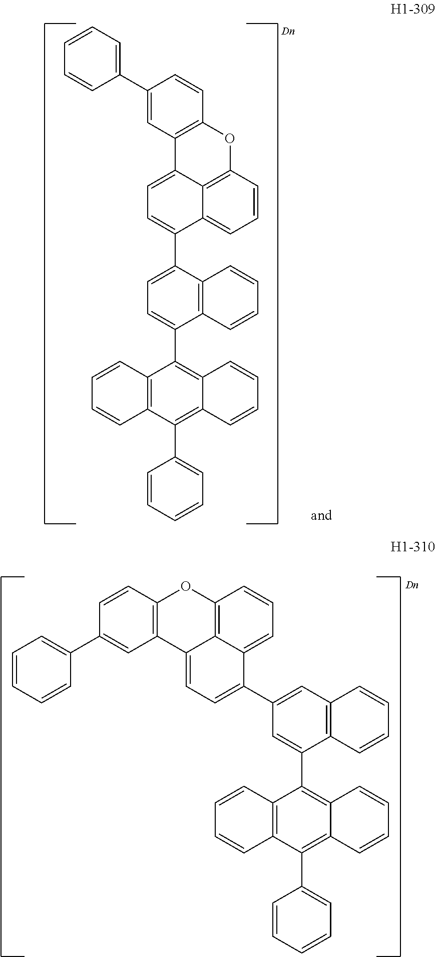
wherein “Dₙ” in the above compounds means that n number of hydrogens is replaced by deuterium, wherein n represents an integer of 1 or more and the upper limit of n is determined by the number of hydrogens that can be substituted in each compound.

15. An organic electroluminescent compound represented by the following Formula 12: 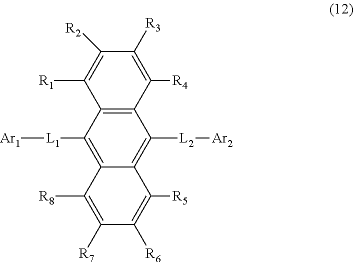
wherein,
R₁ to R₈ each independently represent hydrogen or deuterium;
L₁ and L₂ each independently represent a single bond, a substituted or unsubstituted (C₆-C₃₀)arylene, or a substituted or unsubstituted (3- to 30-membered)heteroarylene;
Ar₁ is represented by the following Formula 1-B or 1-C; 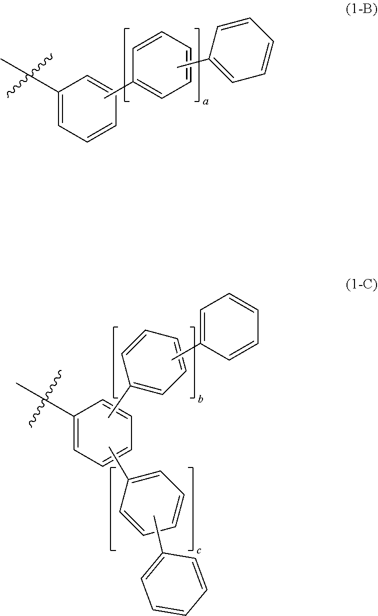
wherein,
a represents an integer of 1 to 3, b and c each independently represent an integer of 0 to 3; [symbol]
represents a linking site to L₁; and
Ar₂ is represented by the following Formula 1-A; 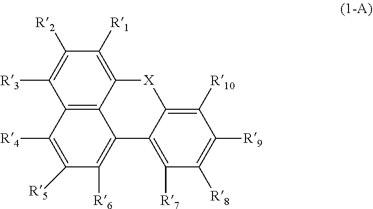
wherein,
X represents —NR″—, —Se—, —O—, or —S—;
R′₁ to R′₁₀ each independently represent hydrogen, deuterium, a substituted or unsubstituted (C₆-C₃₀)aryl, or a substituted or unsubstituted (3- to 30-membered)heteroaryl; or
may be linked to the adjacent substituents to form a substituted or unsubstituted (3- to 30-membered) mono- or polycyclic, alicyclic, aromatic ring, or a combination thereof; and
any one of R′₁ to R′₁₀ is linked to L₂.

16. The organic electroluminescent compound according to claim 15, wherein the organic electroluminescent compound represented by Formula 12 is selected from the following compounds:

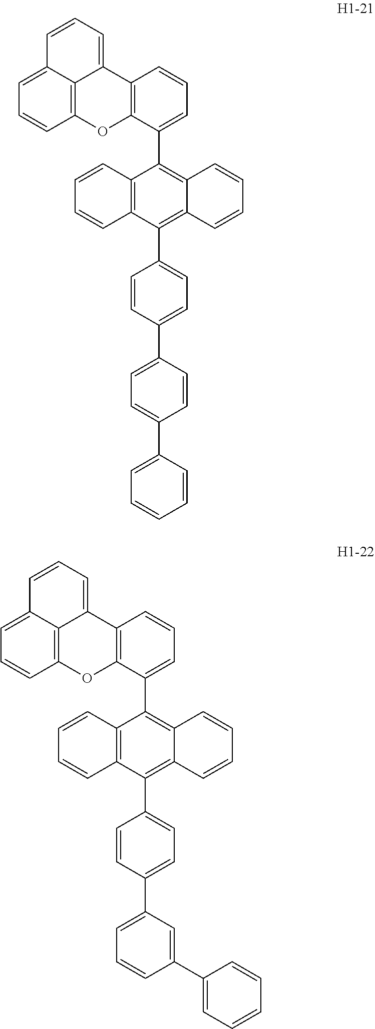 [SMILES: c1ccc(-c2ccc(-c3ccc(-c4c5ccccc5c(-c5cccc6c5Oc5cccc7cccc-6c57)c5ccccc45)cc3)cc2)cc1 ; c1ccc(-c2cccc(-c3ccc(-c4c5ccccc5c(-c5cccc6c5Oc5cccc7cccc-6c57)c5ccccc45)cc3)c2)cc1]

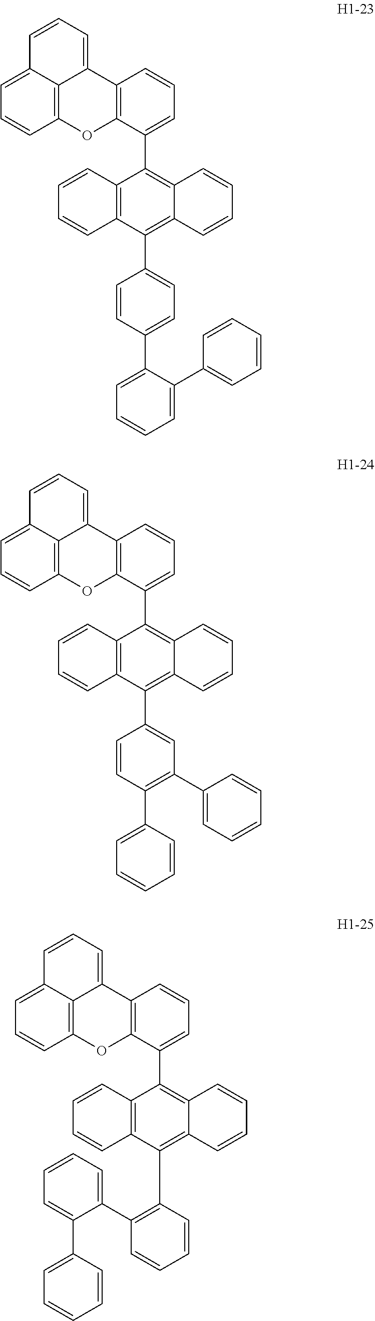 [SMILES: c1ccc(-c2ccccc2-c2ccc(-c3c4ccccc4c(-c4cccc5c4Oc4cccc6cccc-5c46)c4ccccc34)cc2)cc1 ; c1ccc(-c2ccc(-c3c4ccccc4c(-c4cccc5c4Oc4cccc6cccc-5c46)c4ccccc34)cc2-c2ccccc2)cc1 ; c1ccc(-c2ccccc2-c2ccccc2-c2c3ccccc3c(-c3cccc4c3Oc3cccc5cccc-4c35)c3ccccc23)cc1]

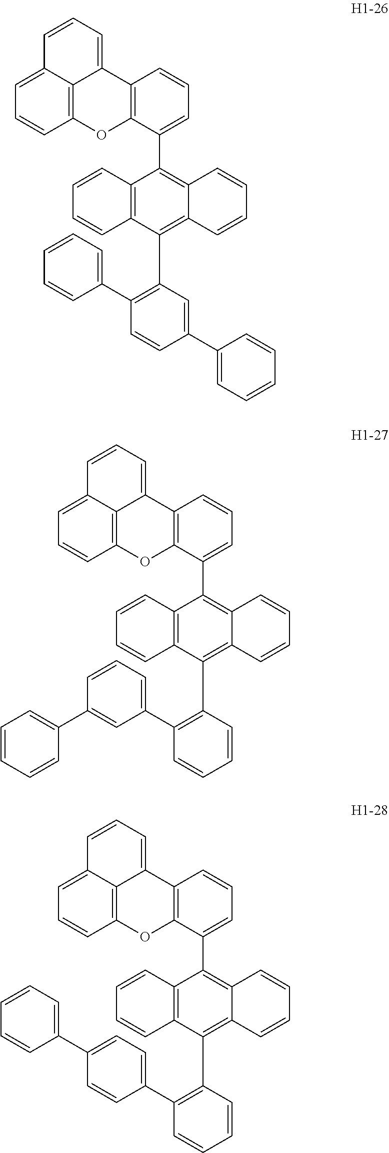 [SMILES: c1ccc(-c2ccc(-c3ccccc3)c(-c3c4ccccc4c(-c4cccc5c4Oc4cccc6cccc-5c46)c4ccccc34)c2)cc1 ; c1ccc(-c2cccc(-c3ccccc3-c3c4ccccc4c(-c4cccc5c4Oc4cccc6cccc-5c46)c4ccccc34)c2)cc1 ; c1ccc(-c2ccc(-c3ccccc3-c3c4ccccc4c(-c4cccc5c4Oc4cccc6cccc-5c46)c4ccccc34)cc2)cc1]

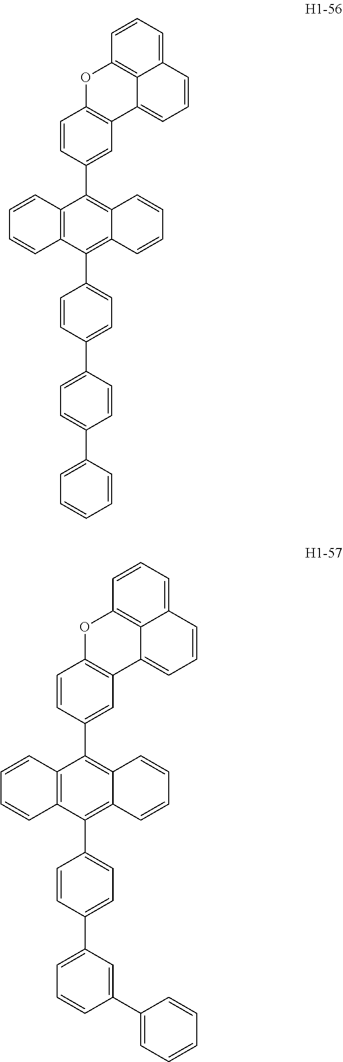 [SMILES: c1ccc(-c2ccc(-c3ccc(-c4c5ccccc5c(-c5ccc6c(c5)-c5cccc7cccc(c57)O6)c5ccccc45)cc3)cc2)cc1 ; c1ccc(-c2cccc(-c3ccc(-c4c5ccccc5c(-c5ccc6c(c5)-c5cccc7cccc(c57)O6)c5ccccc45)cc3)c2)cc1]

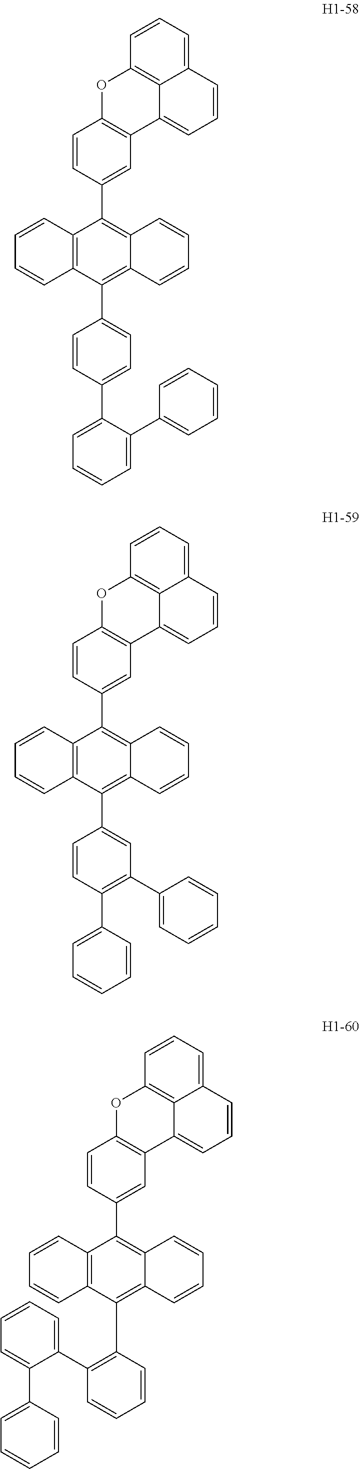 [SMILES: c1ccc(-c2ccccc2-c2ccc(-c3c4ccccc4c(-c4ccc5c(c4)-c4cccc6cccc(c46)O5)c4ccccc34)cc2)cc1 ; c1ccc(-c2ccc(-c3c4ccccc4c(-c4ccc5c(c4)-c4cccc6cccc(c46)O5)c4ccccc34)cc2-c2ccccc2)cc1 ; c1ccc(-c2ccccc2-c2ccccc2-c2c3ccccc3c(-c3ccc4c(c3)-c3cccc5cccc(c35)O4)c3ccccc23)cc1]

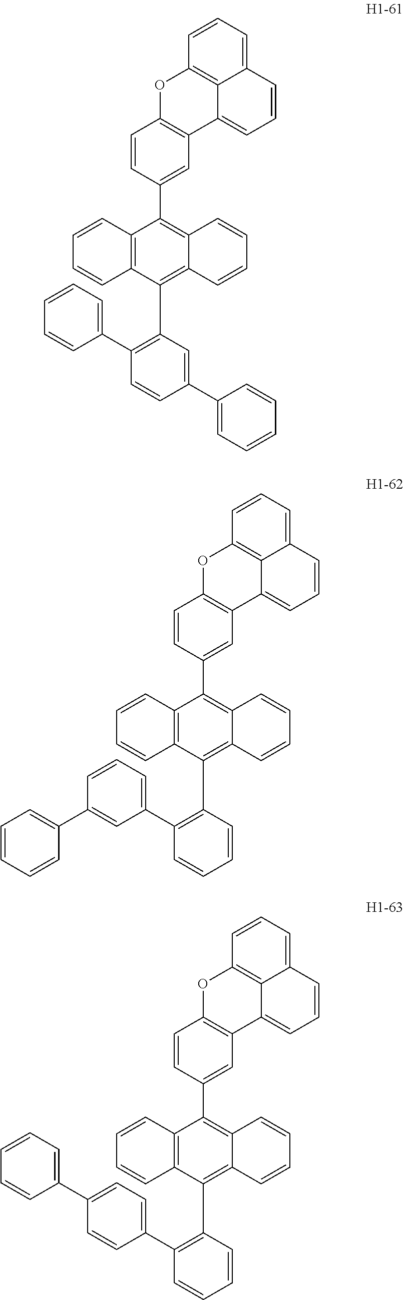

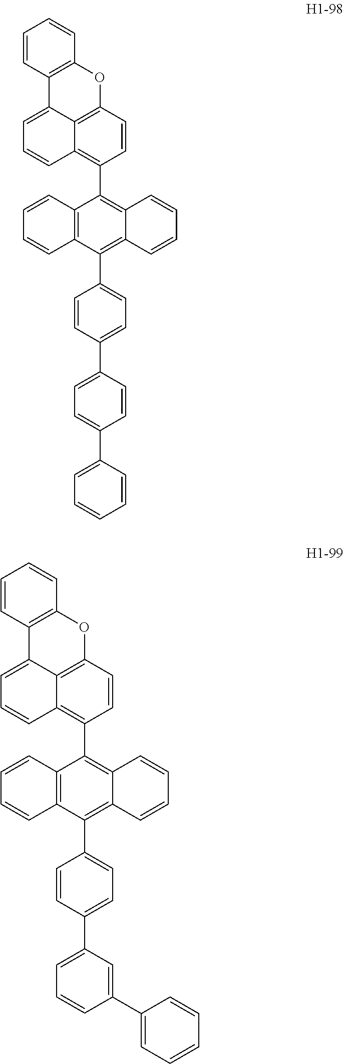 [SMILES: c1ccc(-c2ccc(-c3ccc(-c4c5ccccc5c(-c5ccc6c7c(cccc57)-c5ccccc5O6)c5ccccc45)cc3)cc2)cc1 ; c1ccc(-c2cccc(-c3ccc(-c4c5ccccc5c(-c5ccc6c7c(cccc57)-c5ccccc5O6)c5ccccc45)cc3)c2)cc1]

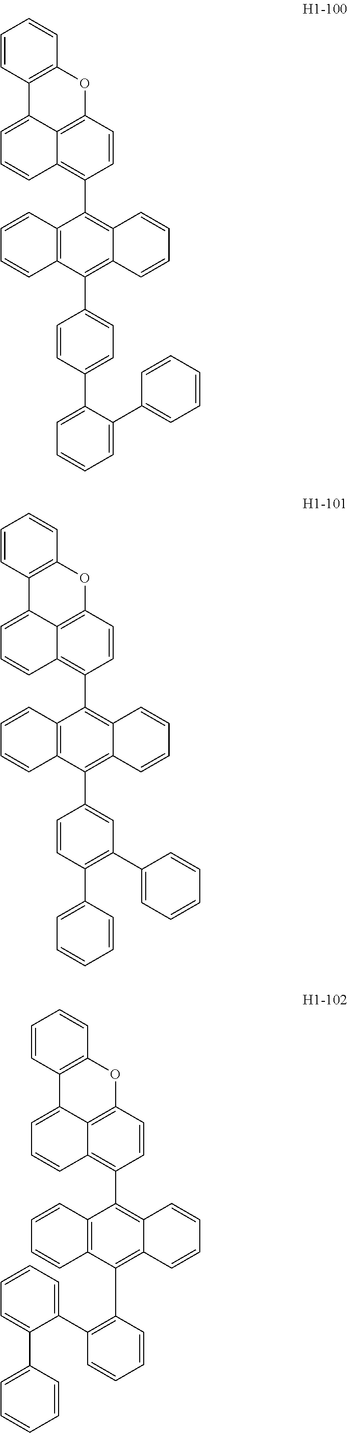 [SMILES: c1ccc(-c2ccccc2-c2ccc(-c3c4ccccc4c(-c4ccc5c6c(cccc46)-c4ccccc4O5)c4ccccc34)cc2)cc1 ; c1ccc(-c2ccc(-c3c4ccccc4c(-c4ccc5c6c(cccc46)-c4ccccc4O5)c4ccccc34)cc2-c2ccccc2)cc1 ; c1ccc(-c2ccccc2-c2ccccc2-c2c3ccccc3c(-c3ccc4c5c(cccc35)-c3ccccc3O4)c3ccccc23)cc1]

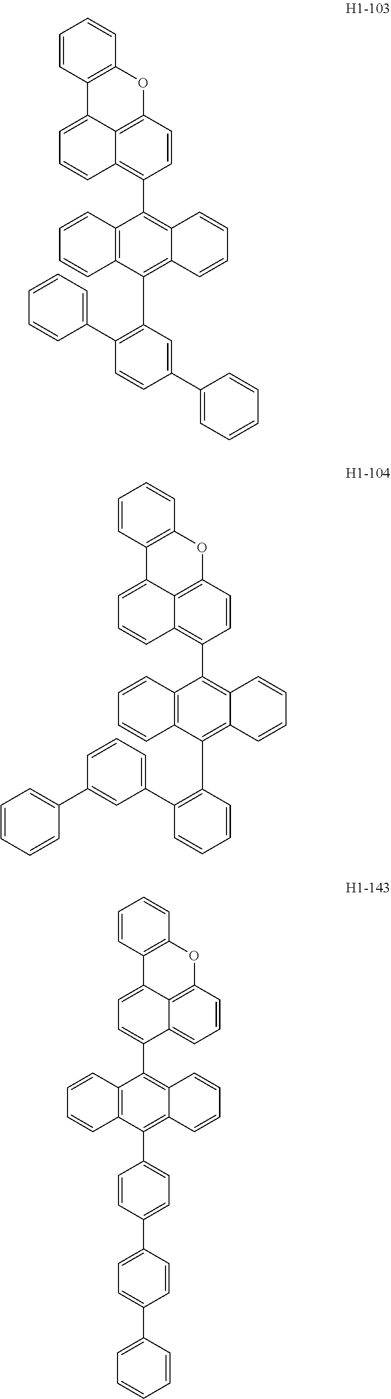 [SMILES: c1ccc(-c2ccc(-c3ccccc3)c(-c3c4ccccc4c(-c4ccc5c6c(cccc46)-c4ccccc4O5)c4ccccc34)c2)cc1 ; c1ccc(-c2cccc(-c3ccccc3-c3c4ccccc4c(-c4ccc5c6c(cccc46)-c4ccccc4O5)c4ccccc34)c2)cc1 ; c1ccc(-c2ccc(-c3ccc(-c4c5ccccc5c(-c5ccc6c7c(cccc57)Oc5ccccc5-6)c5ccccc45)cc3)cc2)cc1]

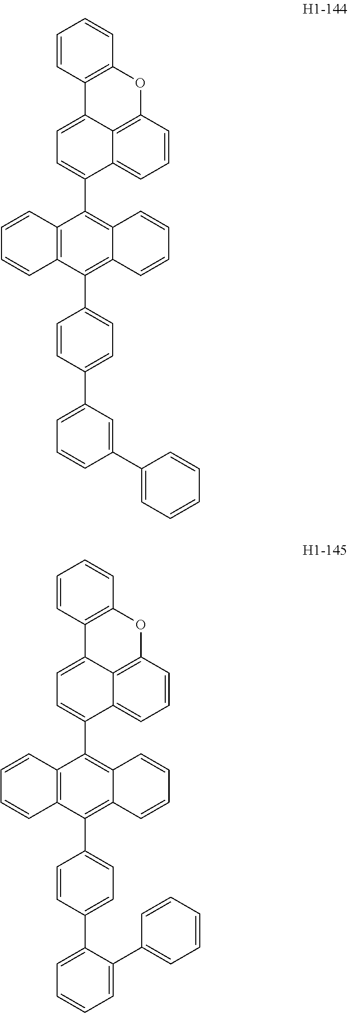 [SMILES: c1ccc(-c2cccc(-c3ccc(-c4c5ccccc5c(-c5ccc6c7c(cccc57)Oc5ccccc5-6)c5ccccc45)cc3)c2)cc1 ; c1ccc(-c2ccccc2-c2ccc(-c3c4ccccc4c(-c4ccc5c6c(cccc46)Oc4ccccc4-5)c4ccccc34)cc2)cc1]

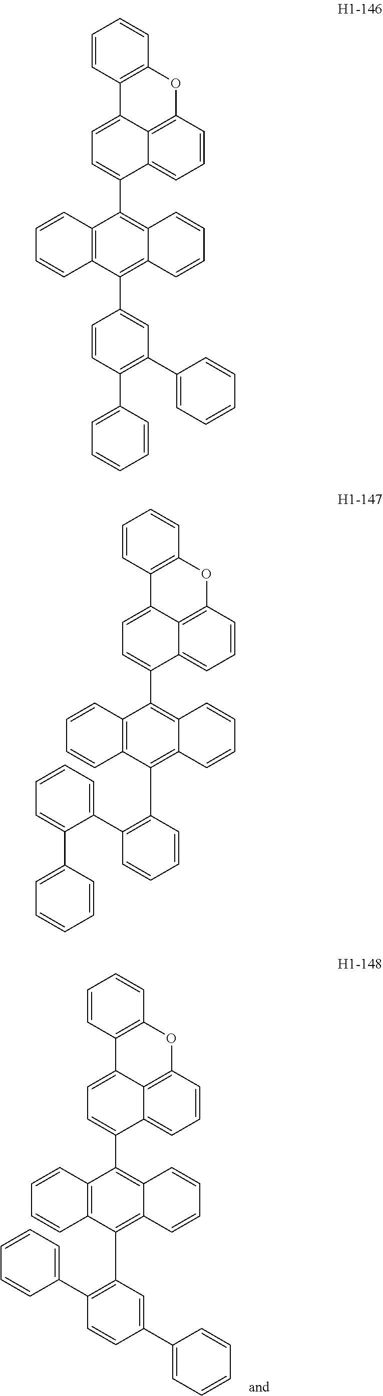 [SMILES: c1ccc(-c2ccc(-c3c4ccccc4c(-c4ccc5c6c(cccc46)Oc4ccccc4-5)c4ccccc34)cc2-c2ccccc2)cc1 ; c1ccc(-c2ccccc2-c2ccccc2-c2c3ccccc3c(-c3ccc4c5c(cccc35)Oc3ccccc3-4)c3ccccc23)cc1 ; c1ccc(-c2ccc(-c3ccccc3)c(-c3c4ccccc4c(-c4ccc5c6c(cccc46)Oc4ccccc4-5)c4ccccc34)c2)cc1]

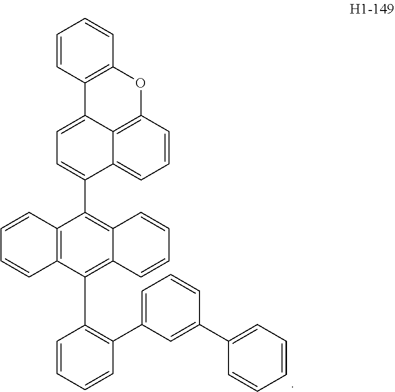 [SMILES: c1ccc(-c2cccc(-c3ccccc3-c3c4ccccc4c(-c4ccc5c6c(cccc46)Oc4ccccc4-5)c4ccccc34)c2)cc1]

17. An organic electroluminescent device comprising the organic electroluminescent compound according to claim 10.

18. An organic electroluminescent device comprising the organic electroluminescent compound according to claim 15.
